(19) United States
(12) Patent Application Publication
(10) Pub. No.: US 2026/0125359 A1
(43) Pub. Date: May 7, 2026

(54) HETEROARYL COMPOUNDS AS MULTI-TARGET PROTEIN KINASE INHIBITORS

(71) Applicant: FUJIAN HAIXI PHARMACEUTICALS CO., LTD., Fuzhou (CN)

(72) Inventors: Ruyong WANG, Fuzhou (CN); Fengsen ZHANG, Fuzhou (CN); Xin LIAN, Fuzhou (CN); Hengdong LI, Fuzhou (CN); Xuan GONG, Fuzhou (CN); Daqiang LIN, Fuzhou (CN); Zhipeng ZHENG, Fuzhou (CN); Xin CHEN, Fuzhou (CN); Xueyin WU, Fuzhou (CN); Hongxue SUN, Fuzhou (CN); Yuxuan ZHANG, Fuzhou (CN); Lili QIU, Fuzhou (CN); Junqing LI, Fuzhou (CN); Guangming CHEN, Fuzhou (CN); Xinshan KANG, Fuzhou (CN)

(21) Appl. No.: 19/433,491

(22) Filed: Dec. 26, 2025

Related U.S. Application Data
(63) Continuation of application No. PCT/CN2024/143464, filed on Dec. 27, 2024.

(30) Foreign Application Priority Data
Dec. 30, 2023 (CN) 202311858456.7
Jun. 18, 2024 (CN) 202410788320.1
Jun. 18, 2024 (CN) 202410788646.4
Jun. 18, 2024 (CN) 202410788666.1

(51) Int. Cl. C07D 401/04; A61K 31/416; A61K 31/437; A61K 31/44; A61K 31/4433; A61K 31/4439; A61K 31/444; A61K 31/4545; A61K 31/4725; A61K 31/496; A61K 31/4965; A61K 31/497; A61K 31/506; A61K 31/519; C07D 213/73; C07D 213/74; C07D 231/56; C07D 241/20; C07D 401/12; C07D 401/14; C07D 403/04; C07D 403/14; C07D 405/12; C07D 405/14; C07D 409/14; C07D 413/12; C07D 413/14; C07D 417/12; C07D 417/14; C07D 471/04; C07D 487/04; C07D 513/04; C07D 519/00

(52) U.S. Cl. CPC C07D 401/04; A61K 31/416; A61K 31/437; A61K 31/44; A61K 31/4433; A61K 31/4439; A61K 31/444; A61K 31/4545; A61K 31/4725; A61K 31/496; A61K 31/4965; A61K 31/497; A61K 31/506; A61K 31/519; C07D 213/73; C07D 213/74; C07D 231/56; C07D 241/20; C07D 401/12; C07D 401/14; C07D 403/04; C07D 403/14; C07D 405/12; C07D 405/14; C07D 409/14; C07D 413/12; C07D 413/14; C07D 417/12; C07D 417/14; C07D 471/04; C07D 487/04; C07D 513/04; C07D 519/00

(57) ABSTRACT

The present disclosure provides compounds of formula (I) having receptor protein tyrosine kinase activity, and a pharmaceutically acceptable salt, stereoisomers, isotopic isomers thereof. The compounds of the present disclosure have high biological activity and can be used to treat diseases or disorders related to the target.

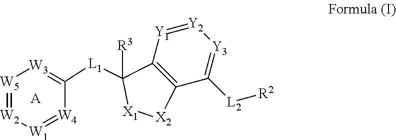

### DECLARATION OF PRIORITY

The present application is a continuation application filed under 35 U.S.C. § 111(a) of PCT Application No. PCT/CN2024/143464, filed on Dec. 27, 2024, which claims priority to, and the benefit of, Chinese Application No. 202410788666.1, filed Jun. 18, 2024, Chinese Application No. 202410788320.1, filed Jun. 18, 2024, Chinese Application No. 202311858456.7, filed Dec. 30, 2023, and Chinese Application No. 202410788646.4, filed Jun. 18, 2024, the contents of each of which are hereby incorporated by reference in their entireties.

### TECHNICAL FIELD

The present disclosure relates to a compound having kinase inhibitory activity, and to its application in the field of medicine. More specifically, the present invention provides heteroaryl compounds having protein tyrosine kinase activity.

### BACKGROUND

Clinical and preclinical evidence demonstrates that TAMs, as key regulatory immune cells, can promote tumor development. Macrophages primarily exist in two polarized states. Alternatively activated M2 subtype TAMs promote tumor progression by secreting anti-inflammatory cytokines (e.g., interleukin (IL)-10, transforming growth factor-s), whereas activated M1 subtype TAMs enhance immune-mediated tumor killing by producing pro-inflammatory cytokines. To overcome the immunosuppressive and tumor-promoting functions of TAMs, current therapeutic strategies focus on the depletion of TAMs in the tumor microenvironment and the reprogramming of TAMs to promote anti-tumor functions (shifting M2 polarization to M1). The CSF-1R kinase is a product encoded by the proto-oncogene c-fms and belongs to the class III receptor tyrosine kinase family, which also includes FMS-like tyrosine kinase 3, stem cell factor receptor, FDGFR, and PDGFR. CSF-1R binds to its ligands, CSF-1 and IL-34, to activate CSF-1R, thereby playing a crucial role in the proliferation, differentiation, and growth of mononuclear phagocytes. In the tumor microenvironment, a large number of macrophages (TAMs) are present, expressing the CSF-1R on their cell surface. When CSF-1 binds to this receptor, macrophages are converted to a pro-tumor state, driving immune suppression and promoting tumor cell growth. Furthermore, high expression of CSF-1R on tumor cells is associated with lower survival rates in certain cancer patients, indicating tumor dependency, making it a potential therapeutic target. By targeting CSF-1R, the development of CSF-1R/CSF-1 inhibitors can effectively reduce the number of TAMs in tumor tissues, promote the generation of anti-tumor macrophages, and help relieve immune suppression. Additionally, it facilitates the infiltration of various immune cells, including T cells and lymphocytes, into the tumor tissue.

Cancer cell growth and metastasis depend on the formation of neovascularization. Vascular endothelial growth factor (VEGF) is the most potent pro-angiogenic factor because solid tumors depend on angiogenesis to provide oxygen and nutrients to aid in their growth and, in turn, as a pathway for invasion and metastasis. In addition, the VEGFa-VEGFR2 signaling pathway plays an important role in the tumor microenvironment and significantly promotes the proliferation and infiltration of Treg (regulatory T cells) in tumor tissues in animal models. Inhibition of the VEGFa-VEGFR2 signaling pathway can inhibit tumor growth by regulating Tregs, MDSCs, and M2 macrophages. Inhibition of VEGFR2 significantly reduced Treg levels in tumor tissues, further helping to deregulate immunosuppression.

PDGFR is a transmembrane glycoprotein with tyrosine kinase activity, primarily expressed on mesenchymal-derived cells such as fibroblasts, pericytes, vascular smooth muscle cells, and stromal stem/progenitor cells. PDGFR consists of a and § subunits. Typically, PDGFRα and PDGFRβ exist as monomers in an autoinhibited state. Upon binding with platelet-derived growth factor (PDGF), PDGFR monomers dimerize, forming isomers PDGFR-αα, PDGFR-αβ, and PDGFR-ββ, which mediate a series of downstream signaling responses. The PDGF/PDGFR primarily promotes angiogenesis and vascular maturation, playing a role in growth and development, and is also closely related to the occurrence and progression of various diseases. Due to the characteristics of tumor microvasculature, such as rapid but immature growth, irregularity, and high permeability, the transport and distribution of chemotherapeutic drugs and oxygen to tumor tissues are hindered, making it easy for tumor cells to metastasize and invade other areas through the leaky vessels. The abnormal vascular system creates a malignant tumor microenvironment characterized by hypoxia, low pH, and elevated interstitial fluid pressure, which interferes with the function of immune cells within the tumor and reduces the efficacy of radiation therapy and chemotherapy. Therefore, the focus of current anti-angiogenic therapy has shifted from simply destroying blood vessels to “normalizing” tumor microvasculature, in order to increase oxygen supply and alter the direction of drug delivery. The root cause of abnormal vascular growth in tumor tissues lies in the disruption of various growth factors within the microenvironment. The PDGF/PDGFR pathway, as a key factor in regulating vascular growth and recruiting pericytes to promote vascular maturation, plays an indispensable role in this process.

Discoidin domain receptor 1 (DDR1) is a novel receptor tyrosine kinase (RTK) that, upon activation, regulates matrix metalloproteinase (MMP), epithelial mesenchymal transition (EMT), and plays an important role in tumor cell migration. Some studies have shown that inhibition of DDR1 expression can induce autophagy and improve the sensitivity of tumor cells to radiation therapy and chemotherapy.

As is well known, multi-target drugs have obvious advantages over combination drugs and multi-component drugs: due to single component, they are better than combination drugs and multi-component drugs in drug metabolism; they overcome the adverse reactions caused by interactions between components; they are convenient to administer, and there is no problem of dosage or ratio in combination drugs; they have predictable pharmacodynamics (PD) and pharmacokinetic (PK) properties; under the same premise of efficacy, synergistic effects can reduce the dosage of drug administration, thus improving the highly selective single-target drugs.) and pharmacokinetic (PK) properties; under the premise of the same therapeutic effect, due to synergistic effect can make the drug dose lower, thus improving the adverse effects of highly selective single-target drugs; slow down the occurrence of drug resistance.

### SUMMARY OF THE INVENTION

The purpose of the present invention is to provide a compound with tyrosine kinase inhibitory activity, which can achieve better tumor suppression effect and reduce the occurrence of drug resistance through multi-target synergistic action.

The present disclosure provides a compound having the structure of formula (I) or a pharmaceutically acceptable salt, stereoisomers, isotopic isomers thereof,

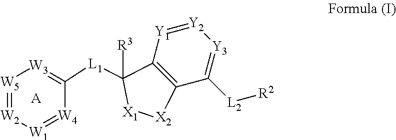

wherein, X₁ represents O, S, NR^{a}, (CR^{L}R^{L′})ₘ, C(O);
wherein, X₂ represents O, S, NR^{a}, (CR^{T}R^{T′})ₙ, C(O);
wherein, W₁ independently represents CR^{W1} or N;
wherein, W₂ independently represents CR^{W2} or N;
wherein, W₃ independently represents CR^{W3} or N;
wherein, W₄ independently represents CR^{W4} or N;
wherein, W₅ independently represents CR¹ or N;
wherein, Y₁ independently represents CR^{Y1} or N;
wherein, Y₂ independently represents CR^{Y2} or N;
wherein, Y₃ independently represents CR^{Y3} or N;
wherein, each R^{W1}, R^{W2}, R^{W3}, R^{W4}, R^{Y1}, R^{Y2}, R^{Y3} independently represents hydrogen, deuterium, C₁-C₆ alkyl, deuterated C₁-C₆ alkyl, halogen, nitro, —OR^{a}, —SR^{a}, —P(O)R^{a}R^{b}, —CN, —S(O)₂R^{a}, —S(O)R^{a}, —SF₅, —NR^{a}R^{b}, —CR^{a}R^{b}NR^{a}R^{b}, —NR^{a}COR^{b}, —CONR^{a}R^{b}, C₁-C₆ haloalkyl, C₁-C₆ haloalkoxy, C₁-C₆ haloalkylthio, hydroxy (C₁-C₆ alkyl); or each R^{W1}, R^{W2}, R^{W3}, R^{W4}, R^{Y1}, R^{Y2}, R^{Y3} independently represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
or W₁ and W₂ together with the atom which they are attached to form a 5-6 membered saturated or unsaturated ring fused to ring A, and the said ring may optionally contain 0, 1, 2 or 3 heteroatoms selected from O, N, or S;
or W₁ and W₄ together with the atom which they are attached to form a 5-6 membered saturated or unsaturated ring fused to ring A, and the said ring may optionally contain 0, 1, 2 or 3 heteroatoms selected from O, N, or S;
wherein, L₁ represents absent, —(CR^{a}R^{b})ₒ—, —CR^{a}R^{b}O—, —OCR^{a}R^{b}—, —O—, —S—, —S(O)₂—, —C(O)—, —NR^{a}—, —S(O)₂NR^{a}—, —OC(O)—, —C(O)O—, —NR^{a}S(O)₂—, —CR^{a}R^{b}NR^{a}—, or —NR^{a}CR^{a}R^{b}—;
wherein, L₂ represents absent, —(CR^{a}R^{b})ₒ—, —CR^{a}R^{b}O—, —OCR^{a}R^{b}—, —O—, —C(O)—, —NR^{a}—, —S(O)₂NR^{a}—, —OC(O)—, —C(O)O—, —NR^{a}S(O)₂—, —CR^{a}R^{b}NR^{a}—, —NR^{a}CR^{a}R^{b}—, —NR^{a}C(O)NR^{b}—, —NR^{a}C(O)NR^{b}CR^{a}R^{b}—, —NR^{a}C(O)O—, —NR^{a}C(O)—, or —C(O)NR^{a}—;
Wherein, R¹ represents hydrogen, deuterium, C₁-C₆ alkyl, deuterated C₁-C₆ alkyl, halogen, —OR^{a}, —SR^{a}, —P(O)R^{a}R^{b}, —CN, —S(O)₂R^{a}, —S(O)R^{a}, —SF₅, —NR^{a}R^{b}, C₁-C₆ haloalkyl, C₁-C₆ haloalkoxy, C₁-C₆ haloalkylthio, hydroxy (C₁-C₆ alkyl); or R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl, which are substituted by 0-4 substituents that selected from deuterium, halogen, C₁-C₆ alkyl, C₃-C₆ heterocycloalkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}; and when R¹ is a ring, any two adjacent substituents on the ring may combine to form a ring, and the said ring may optionally contain 0, 1 or 2 heteroatoms selected from O, N, or S; further, the said ring may optionally be substituted by halogen, hydroxy or C₁-C₆ alkyl;
or W₅ and W₂ together with the atom which they are attached to form a 5-6 membered saturated or unsaturated ring fused to ring A, and the said ring may optionally contain 0, 1, 2 or 3 heteroatoms selected from O, N, or S; and further, the said ring may optionally substituted by 0, 1 or 2 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, R² represents C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl, —CR^{a}R^{b}C₆-C₁₀ aryl, —CR^{a}R^{b}(5-10 membered heteroaryl); which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, C₆-C₁₀ aryl, or 5-10 membered heteroaryl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, C₃-C₆ halocycloalkyl, C₃-C₆ haloheterocycloalkyl, C₁-C₆ alkyl-substituted C₃-C₆ heterocycloalkyl, —SO₃R^{a}, —SR^{a}, —S(O)₂R^{a}, —S(O)R^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}; and when R² is a ring, any two adjacent substituents on the ring may combine to form a ring, and the said ring may optionally contain 0, 1 or 2 heteroatoms selected from O, N, and S; and further, the said ring may optionally be substituted by halogen, hydroxy or C₁-C₆ alkyl;
wherein, R³ represents hydrogen, halogen, C₁-C₆ haloalkyl, C₁-C₆ alkyl, or hydroxy;
or R³ and W₃ together with the atom which they are attached to form a 5-7 membered saturated or unsaturated ring fused to ring A, and the said ring may optionally contain 0, 1 or 2 heteroatoms selected from O, N, or S; and further, the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, each R^{a} and R^{b} independently represents hydrogen, deuterium, halogen, C₁-C₆ alkyl, C₁-C₆ haloalkyl, hydroxy (C₁-C₆ alkyl), (CH₃)₂N—(C₁-C₆ alkyl), C₃-C₁₀ cycloalkyl; or R^{a} and R^{b} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, each R^{L} and R^{L′} independently represents hydrogen, deuterium, halogen, C₁-C₆ alkyl, C₁-C₆ haloalkyl, hydroxy (C₁-C₆ alkyl), C₃-C₁₀ cycloalkyl; or R^{L} and R^{L′} together with the carbon atom which they are attached to form a 3-6 membered ring, and the said ring may optionally contain 0, 1 or 2 heteroatoms selected from O, N, or S; and further, the said ring may be optionally substituted by halogen, hydroxy or C₁-C₆ alkyl;
wherein, each R^{T} and R^{T′} independently represents hydrogen, deuterium, halogen, C₁-C₆ alkyl, C₁-C₆ haloalkyl, hydroxy (C₁-C₆ alkyl), C₃-C₁₀ cycloalkyl; or R^{T} and R^{T′} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, o, m, n represents the integer of 1, 2 or 3.

In some embodiments of the present disclosure, the compound has a structure as shown in formula (I):

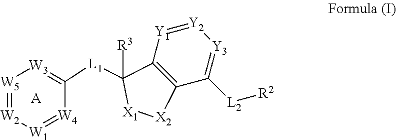

wherein, X₁ represents O, S, NR^{a}, (CR^{L}R^{L′})ₘ, C(O);
wherein, X₂ represents O, S, NR^{a}, (CR^{T}R^{T′})ₙ, C(O);
wherein, W₁ independently represents CR^{W1} or N;
wherein, W₂ independently represents CR^{W2} or N;
wherein, W₃ independently represents CR^{W3} or N;
wherein, W₄ independently represents CR^{W4} or N;
wherein, W₅ independently represents CR¹ or N;
wherein, Y₁ independently represents CR^{Y1} or N;
wherein, Y₂ independently represents CR^{Y2} or N;
wherein, Y₃ independently represents CR^{Y3} or N;
wherein, each R^{W1}, R^{W2}, R^{W3}, R^{W4}, R^{Y1}, R^{Y2}, R^{Y3} independently represents hydrogen, deuterium, C₁-C₆ alkyl, deuterated C₁-C₆ alkyl, halogen, nitro, —OR^{a}, —SR^{a}, —P(O)R^{a}R^{b}, —CN, —S(O)₂R^{a}, —S(O)R^{a}, —SF₅, —NR^{a}R^{b}, —CR^{a}R^{b}NR^{a}R^{b}, —NR^{a}COR^{b}, —CONR^{a}R^{b}, C₁-C₆ haloalkyl, C₁-C₆ haloalkoxy, C₁-C₆ haloalkylthio, hydroxy (C₁-C₆ alkyl); or each R^{W1}, R^{W2}, R^{W3}, R^{W4}, R^{Y1}, R^{Y2}, R^{Y3} independently represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
or W₁ and W₂ together with the atom which they are attached to form a 5-6 membered saturated or unsaturated ring fused to ring A, and the said ring may optionally contain 0, 1, 2 or 3 heteroatoms selected from O, N, or S;
or W₁ and W₄ together with the atom which they are attached to form a 5-6 membered saturated or unsaturated ring fused to ring A, and the said ring may optionally contain 0, 1, 2 or 3 heteroatoms selected from O, N, or S;
wherein, L₁ represents absent, —(CR^{a}R^{b})ₒ—, —CR^{a}R^{b}O—, —OCR^{a}R^{b}—, —O—, —S—, —S(O)₂—, —C(O)—, —NR^{a}—, —S(O)₂NR^{a}—, —OC(O)—, —C(O)O—, —NR^{a}S(O)₂—, —CR^{a}R^{b}NR^{a}—, or —NR^{a}CR^{a}R^{b}—;
wherein, L₂ represents absent, —(CR^{a}R^{b})ₒ—, —CR^{a}R^{b}O—, —OCR^{a}R^{b}—, —O—, —C(O)—, —NR^{a}—, —S(O)₂NR^{a}—, —OC(O)—, —C(O)O—, —NR^{a}S(O)₂—, —CR^{a}R^{b}NR^{a}—, —NR^{a}CR^{a}R^{b}—, —NR^{a}C(O)NR^{b}—, —NR^{a}C(O)NR^{b}CR^{a}R^{b}—, —NR^{a}C(O)O—, —NR^{a}C(O)—, or —C(O)NR^{a}—;
Wherein, R¹ represents hydrogen, deuterium, C₁-C₆ alkyl, deuterated C₁-C₆ alkyl, halogen, —OR^{a}, —SR^{a}, —P(O)R^{a}R^{b}, —CN, —S(O)₂R^{a}, —S(O)R^{a}, —SF₅, —NR^{a}R^{b}, —CR^{a}R^{b}NR^{a}R^{b}, C₁-C₆ haloalkyl, C₁-C₆ haloalkoxy, C₁-C₆ haloalkylthio, hydroxy (C₁-C₆ alkyl); or R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl, which are substituted by 0-4 substituents that selected from deuterium, halogen, C₁-C₆ alkyl, C₃-C₆ heterocycloalkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
or W₅ and W₂ together with the atom which they are attached to form a 5-6 membered saturated or unsaturated ring fused to ring A, and the said ring may optionally contain 0, 1 or 2 heteroatoms selected from O, N, or S; and further, the said ring may optionally substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, R² represents C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, —CR^{a}R^{b}C₆-C₁₀ aryl, —CR^{a}R^{b}(5-10 membered heteroaryl); which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, C₆-C₁₀ aryl, or 5-10 membered heteroaryl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —S(O)₂R^{a}, —S(O)R^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, R³ represents hydrogen, halogen, C₁-C₆ haloalkyl, C₁-C₆ alkyl, or hydroxy;
or R³ and W₃ together with the atom which they are attached to form a 5-7 membered saturated or unsaturated ring fused to ring A, and the said ring may optionally contain 0, 1 or 2 heteroatoms selected from O, N, or S; and further, the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, each R^{a} and R^{b} independently represents hydrogen, deuterium, halogen, C₁-C₆ alkyl, C₁-C₆ haloalkyl, hydroxy (C₁-C₆ alkyl), C₃-C₁₀ cycloalkyl; or R^{a} and R^{b} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, each R^{L} and R^{L′} independently represents hydrogen, deuterium, halogen, C₁-C₆ alkyl, C₁-C₆ haloalkyl, hydroxy (C₁-C₆ alkyl), C₃-C₁₀ cycloalkyl; or R^{L} and R^{L′} together with the carbon atom which they are attached to form a 3-6 membered ring, and the said ring may optionally contain 0, 1 or 2 heteroatoms selected from O, N, or S; and further, the said ring may be optionally substituted by halogen, hydroxy or C₁-C₆ alkyl;
wherein, each R^{T} and R^{T′} independently represents hydrogen, deuterium, halogen, C₁-C₆ alkyl, C₁-C₆ haloalkyl, hydroxy (C₁-C₆ alkyl), C₃-C₁₀ cycloalkyl; or R^{T} and R^{T′} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, o, m, n represents the integer of 1, 2 or 3.

In some embodiments of the present disclosure, the compound has a structure as shown in formula (I):

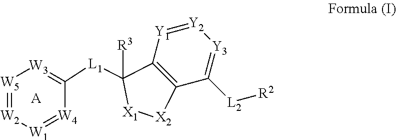

wherein, X₁ represents O, S, NR^{a}, (CR^{L}R^{L′})ₘ, C(O);
wherein, X₂ represents O, S, NR^{a}, (CR^{T}R^{T′})ₙ, C(O);
wherein, W₁ independently represents CR^{W1} or N;
wherein, W₂ independently represents CR^{W2} or N;
wherein, W₃ independently represents CR^{W3} or N;
wherein, W₄ independently represents CR^{W4} or N;
wherein, W₅ independently represents CR¹ or N;
wherein, Y₁ independently represents CR^{Y1} or N;
wherein, Y₂ independently represents CR^{Y2} or N;
wherein, Y₃ independently represents CR^{Y3} or N;
wherein, each R^{W1}, R^{W2}, R^{W3}, R^{W4}, R^{Y1}, R^{Y2}, R^{Y3} independently represents hydrogen, deuterium, C₁-C₆ alkyl, deuterated C₁-C₆ alkyl, halogen, nitro, —OR^{a}, —SR^{a}, —P(O)R^{a}R^{b}, —CN, —S(O)₂R^{a}, —S(O)R^{a}, —SF₅, —NR^{a}R^{b}, —CR^{a}R^{b}NR^{a}R^{b}, —NR^{a}COR^{b}, —CONR^{a}R^{b}, C₁-C₆ haloalkyl, C₁-C₆ haloalkoxy, C₁-C₆ haloalkylthio, hydroxy (C₁-C₆alkyl); or each R^{W1}, R^{W2}, R^{W3}, R^{W4}, R^{Y1}, R^{Y2}, R^{Y3} independently represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
or W₁ and W₂ together with the atom which they are attached to form a 5-6 membered saturated or unsaturated ring fused to ring A, and the said ring may optionally contain 0, 1, 2 or 3 heteroatoms selected from O, N, or S;
or W₁ and W₄ together with the atom which they are attached to form a 5-6 membered saturated or unsaturated ring fused to ring A, and the said ring may optionally contain 0, 1, 2 or 3 heteroatoms selected from O, N, or S;
wherein, L₁ represents absent, —(CR^{a}R^{b})ₒ—, —CR^{a}R^{b}O—, —OCR^{a}R^{b}—, —O—, —S—, —S(O)₂—, —C(O)—, —NR^{a}—, —S(O)₂NR^{a}—, —OC(O)—, —C(O)O—, —NR^{a}S(O)₂—, —CR^{a}R^{b}NR^{a}—, or —NR^{a}CR^{a}R^{b}—;
wherein, L₂ represents absent, —(CR^{a}R^{b})ₒ—, —CR^{a}R^{b}O—, —OCR^{a}R^{b}—, —O—, —C(O)—, —NR^{a}—, —S(O)₂NR^{a}—, —OC(O)—, —C(O)O—, —NR^{a}S(O)₂—, —CR^{a}R^{b}NR^{a}—, —NR^{a}CR^{a}R^{b}—, —NR^{a}C(O)NR^{b}—, —NR^{a}C(O)NR^{b}CR^{a}R^{b}—, —NR^{a}C(O)O—, —NR^{a}C(O)—, or —C(O)NR^{a}—;
Wherein, R¹ represents hydrogen, deuterium, C₁-C₆ alkyl, deuterated C₁-C₆ alkyl, halogen, —OR^{a}, —SR^{a}, —P(O)R^{a}R^{b}, —CN, —S(O)₂R^{a}, —S(O)R^{a}, —SF₅, —NR^{a}R^{b}, C₁-C₆ haloalkyl, C₁-C₆ haloalkoxy, C₁-C₆ haloalkylthio, hydroxy (C₁-C₆ alkyl); or R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl, which are substituted by 0-4 substituents that selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
or W₅ and W₂ together with the atom which they are attached to form a 5-6 membered saturated or unsaturated ring fused to ring A. and the said ring may optionally contain 0, 1 or 2 heteroatoms selected from O, N, or S; and further, the said ring may optionally substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}; wherein, R² represents C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, —CR^{a}R^{b}C₆-C₁₀ aryl, —CR^{a}R^{b} (5-10 membered heteroaryl); which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, C₆-C₁₀ aryl, or 5-10 membered heteroaryl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —S(O)₂R^{a}, —S(O)R^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, R³ represents hydrogen, halogen, C₁-C₆ haloalkyl, C₁-C₆ alkyl, or hydroxy;
or R³ and W₃ together with the atom which they are attached to form a 5-7 membered saturated or unsaturated ring fused to ring A, and the said ring may optionally contain 0, 1 or 2 heteroatoms selected from O, N, or S; and further, the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, each R^{a} and R^{b} independently represents hydrogen, deuterium, halogen, C₁-C₆ alkyl, C₁-C₆ haloalkyl, hydroxy (C₁-C₆ alkyl), C₃-C₁₀ cycloalkyl; or R^{a} and R^{b} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, each R^{L} and R^{L′} independently represents hydrogen, deuterium, halogen, C₁-C₆ alkyl, C₁-C₆ haloalkyl, hydroxy (C₁-C₆ alkyl), C₃-C₁₀ cycloalkyl; or R^{L} and R^{L′} together with the carbon atom which they are attached to form a 3-6 membered ring, and the said ring may optionally contain 0, 1 or 2 heteroatoms selected from O, N, or S; and further, the said ring may be optionally substituted by halogen, hydroxy or C₁-C₆ alkyl;
wherein, each R^{T} and R^{T′} independently represents hydrogen, deuterium, halogen, C₁-C₆ alkyl, C₁-C₆ haloalkyl, hydroxy (C₁-C₆ alkyl), C₃-C₁₀ cycloalkyl; or R^{T} and R^{T′} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, o, m, n represents the integer of 1, 2 or 3.

In some embodiments of the present disclosure, wherein W₁ represents CH, CCH₃, CF, CCF₃, CCHF₂, CCH₂F, C(OCH₃), C(CN) or N.

In some embodiments of the present disclosure, wherein W₁ represents N.

In some embodiments of the present disclosure, wherein W₂ represents CH, CCH₃, CF, CCF₃, CCHF₂, CCH₂F, C(OCH₃), CNH₂, CNHCH₃, CN(CH₃)₂, CCH₂NHCH₃, CCH₂N(CH₃)₂, C(CN) or N.

In some embodiments of the present disclosure, wherein W₃ represents CH, CCH₃, CF, CCF₃, CCHF₂, CCH₂F, C(OCH₃), C(CN) or N.

In some embodiments of the present disclosure, wherein W₄ represents CH, CCH₃, CF, CCF₃, CCHF₂, CCH₂F, C(OCH₃), CNH₂, CNHCH₃, C(CN) or N.

In some embodiments of the present disclosure, wherein Y₁ represents CH, CCH₃, CF, CCl, CCF₃, CCHF₂, CCH₂F, C(OCH₃), C(CN) or N.

In some embodiments of the present disclosure, wherein Y₂ represents CH, CCH₃, CF, CCl, CCF₃, CCHF₂, CCH₂F, C(OCH₃), C(CN) or N.

In some embodiments of the present disclosure, wherein Y₃ represents CH, CCH₃, CF, CCl, CCF₃, CCHF₂, CCH₂F, C(OCH₃), C(CN) or N.

In some embodiments of the present disclosure, wherein X₁ represents —CH₂—, —CF₂—, —CHF—, —O—, —S—, —CHOH—, —CH₂CH₂— or —C(CH₃)₂—.

In some embodiments of the present disclosure, wherein X₂ represents —CH₂—, —CF₂—, —CHF—, —O—, —S—, —CHOH—, —CH₂CH₂— or —C(CH₃)₂—.

In some embodiments of the present disclosure, wherein L₁ represents —CH₂—, —CHF—, —CF₂—, —O—, —NH— or —NCH₃—.

In some embodiments of the present disclosure, wherein L₂ represents —NHC(O)—, —C(O)NH—, —NHC(O)NH—, —NHC(O)O—, —CH₂NH—, —NHCH₂—, —CH₂O—, —OCH₂— or —NH—.

In some embodiments of the present disclosure, wherein R¹ represents C₁-C₆ alkyl, halogen, —OH, O(C₁-C₆ alkyl); or R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, —OR^{a}, hydroxy (C₁-C₆ alkyl), —CN or C₁-C₆ haloalkyl.

In some embodiments of the present disclosure, wherein R¹ represents 5-10 membered heteroaryl, which are substituted by 0-4 substituents selected from deuterium, halogen, C₁-C₆ alkyl, —OR^{a}, hydroxy (C₁-C₆ alkyl), —CN or C₁-C₆ haloalkyl.

In some embodiments of the present disclosure, wherein R¹ represents pyrazolyl, triazolyl, oxazolyl, thiazolyl, imidazolyl, which are substituted by 0-4 substituents selected from deuterium, halogen, C₁-C₆ alkyl, —OR^{a}, hydroxy (C₁-C₆ alkyl), —CN or C₁-C₆ haloalkyl.

In some embodiments of the present disclosure, wherein R¹ represent

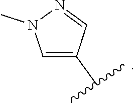 [SMILES: *c1cnn(C)c1]

In some embodiments of the present disclosure, wherein R² represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, phenyl, 5-6 membered heteroaryl, —OR^{a}, oxo, hydroxy (C₁-C₆alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein W₅ and W₂ combine to form a ring fused to ring A having the following structure:

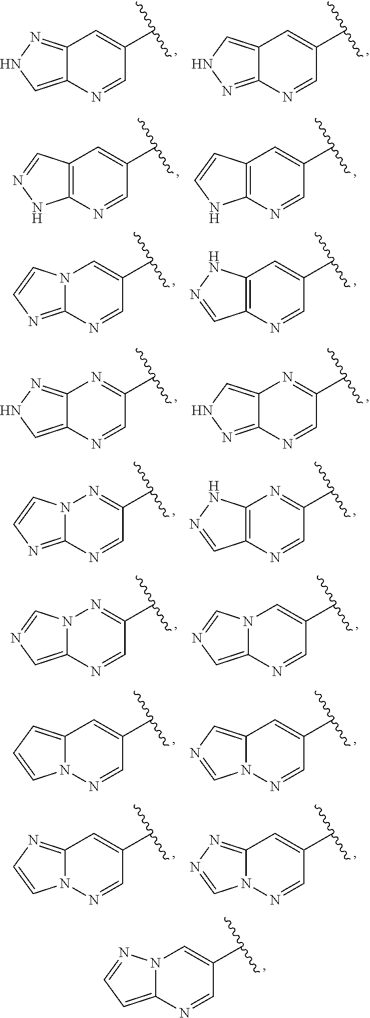

wherein the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}; and wherein the wavy line indicates the point of attachment with L₁.

In some embodiments of the present disclosure, wherein W₅ and W₂ combine to form a ring fused to ring A having the following structure:

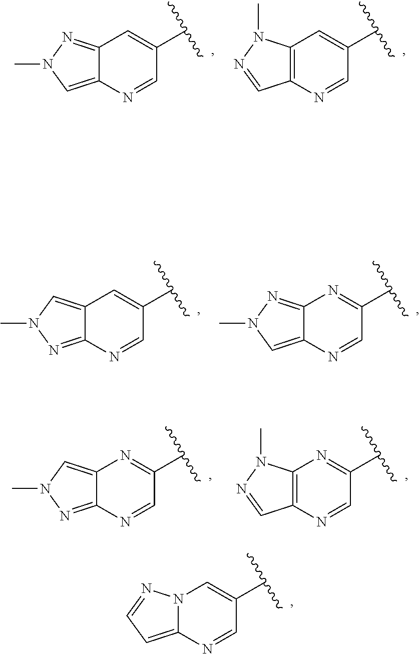

wherein the wavy line indicates the point of attachment with L₁.

In some embodiments of the present disclosure, wherein R² represents phenyl, pyridinyl, indazolyl, indolyl, thiazolyl, isothiazolyl, imidazolyl, pyrazolyl, oxazolyl, isoxazolyl, 1,2,4-oxadiazolyl, 1,3,4-oxadiazolyl, pyridazinyl, piperazinyl, pyrazinyl, cyclohexyl, benzothienyl, 1,2-benzoisoxazolyl, benzo[d] isothiazolyl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, phenyl, 5-6 membered heteroaryl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R² represents

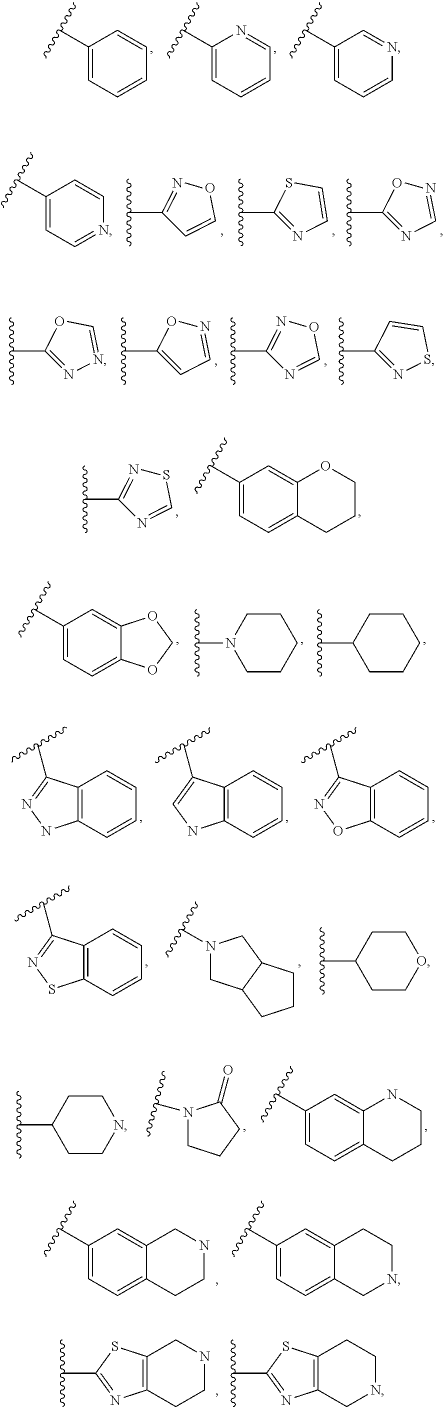

which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R² represents:

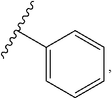 [SMILES: *c1ccccc1]

which is substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R³ represents hydrogen or CH₃.

The present disclosure provides a compound having the structure of formula (I) or a pharmaceutically acceptable salt, stereoisomers, isotopic isomers thereof,

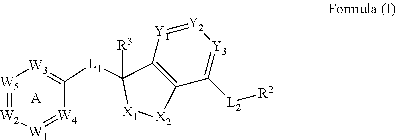

wherein, X₁ represents O, S, NR^{a}, (CR^{L}R^{L′})ₘ, C(O);
wherein, X₂ represents O, S, NR^{a}, (CR^{T}R^{T′})ₙ, C(O);
wherein, W₁ independently represents CR^{W1} or N;
wherein, W₂ independently represents CR^{W2} or N;
wherein, W₃ independently represents CR^{W3} or N;
wherein, W₄ independently represents CR^{W4} or N;
wherein, W₅ independently represents N;
wherein, Y₁ independently represents CR^{Y1} or N;
wherein, Y₂ independently represents CR^{Y2} or N;
wherein, Y₃ independently represents CR^{Y3} or N;
wherein, each R^{W1}, R^{W2}, R^{W3}, R^{W4}, R^{Y1}, R^{Y2}, R^{Y3} independently represents hydrogen, deuterium, C₁-C₆ alkyl, deuterated C₁-C₆ alkyl, halogen, nitro, —OR^{a}, —SR^{a}, —P(O)R^{a}R^{b}, —CN, —S(O)₂R^{a}, —S(O)R^{a}, —SF₅, —NR^{a}R^{b}, —CR^{a}R^{b}NR^{a}R^{b}, —NR^{a}COR^{b}, —CONR^{a}R^{b}, C₁-C₆ haloalkyl, C₁-C₆ haloalkoxy, C₁-C₆ haloalkylthio, hydroxy (C₁-C₆alkyl); or each R^{W1}, R^{W2}, R^{W3}, R^{W4}, R^{Y1}, R^{Y2}, R^{Y3} independently represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
or W₁ and W₂ together with the atom which they are attached to form a 5-6 membered saturated or unsaturated ring fused to ring A, and the said ring may optionally contain 0, 1, 2 or 3 heteroatoms selected from O, N, or S;
or W₁ and W₄ together with the atom which they are attached to form a 5-6 membered saturated or unsaturated ring fused to ring A, and the said ring may optionally contain 0, 1, 2 or 3 heteroatoms selected from O, N, or S;
wherein, L₁ represents absent, —(CR^{a}R^{b})ₒ—, —CR^{a}R^{b}O—, —OCR^{a}R^{b}—, —O—, —S—, —S(O)₂—, —C(O)—, —NR^{a}—, —S(O)₂NR^{a}—, —OC(O)—, —C(O)O—, —NR^{a}S(O)₂—, —CR^{a}R^{b}NR^{a}—, or —NR^{a}CR^{a}R^{b}—;
wherein, L₂ represents absent, —(CR^{a}R^{b})ₒ—, —CR^{a}R^{b}O—, —OCR^{a}R^{b}—, —O—, —C(O)—, —NR^{a}—, —S(O)₂NR^{a}—, —OC(O)—, —C(O)O—, —NR^{a}S(O)₂—, —CR^{a}R^{b}NR^{a}—, —NR^{a}CR^{a}R^{b}—, —NR^{a}C(O)NR^{b}—, —NR^{a}C(O)NR^{b}CR^{a}R^{b}—, —NR^{a}C(O)O—, —NR^{a}C(O)—, or —C(O)NR^{a}—;
or W₅ and W₂ together with the atom which they are attached to form a 5-6 membered saturated or unsaturated ring fused to ring A, and the said ring may optionally contain 0, 1 or 2 heteroatoms selected from O, N, or S; and further, the said ring may optionally substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, R² represents C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl, —CR^{a}R^{b}C₆-C₁₀ aryl, —CR^{a}R^{b}(5-10 membered heteroaryl); which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, C₆-C₁₀ aryl, 5-10 membered heteroaryl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, R³ represents hydrogen, halogen, C₁-C₆ haloalkyl, C₁-C₆ alkyl, or hydroxy;
or R³ and W₃ together with the atom which they are attached to form a 5-7 membered saturated or unsaturated ring fused to ring A, and the said ring may optionally contain 0, 1 or 2 heteroatoms selected from O, N, or S; and further, the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, each R^{a} and R^{b} independently represents hydrogen, deuterium, halogen, C₁-C₆ alkyl, C₁-C₆ haloalkyl, hydroxy (C₁-C₆ alkyl), C₃-C₁₀ cycloalkyl; or R^{a} and R^{b} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, each R^{L} and R^{L′} independently represents hydrogen, deuterium, halogen, C₁-C₆ alkyl, C₁-C₆ haloalkyl, hydroxy (C₁-C₆ alkyl), C₃-C₁₀ cycloalkyl; or R^{L} and R^{L′} together with the carbon atom which they are attached to form a 3-6 membered ring, and the said ring may optionally contain 0, 1 or 2 heteroatoms selected from O, N, or S; and further, the said ring may be optionally substituted by halogen, hydroxy or C₁-C₆ alkyl;
wherein, each R^{T} and R^{T′} independently represents hydrogen, deuterium, halogen, C₁-C₆ alkyl, C₁-C₆ haloalkyl, hydroxy (C₁-C₆ alkyl), C₃-C₁₀ cycloalkyl; or R^{T} and R^{T′} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, o, m, n represents the integer of 1, 2 or 3.

In some embodiments of the present disclosure, wherein W₁ represents CH, CCH₃, CF, CCF₃, CCHF₂, CCH₂F, C(OCH₃), C(CN) or N.

In some embodiments of the present disclosure, wherein W₁ represents N.

In some embodiments of the present disclosure, wherein W₂ represents CH, CCH₃, CF, CCF₃, CCHF₂, CCH₂F, C(OCH₃), CNH₂, CNHCH₃, CN(CH₃)₂, CCH₂NHCH₃, CCH₂N(CH₃)₂, C(CN) or N.

In some embodiments of the present disclosure, wherein W₃ represents CH, CCH₃, CF, CCF₃, CCHF₂, CCH₂F, C(OCH₃), C(CN) or N.

In some embodiments of the present disclosure, wherein W₄ represents CH, CCH₃, CF, CCF₃, CCHF₂, CCH₂F, C(OCH₃), CNH₂, CNHCH₃, C(CN) or N.

In some embodiments of the present disclosure, wherein Y₁ represents CH, CCH₃, CF, CCl, CCF₃, CCHF₂, CCH₂F, C(OCH₃), C(CN) or N.

In some embodiments of the present disclosure, wherein Y₂ represents CH, CCH₃, CF, CCl, CCF₃, CCHF₂, CCH₂F, C(OCH₃), C(CN) or N.

In some embodiments of the present disclosure, wherein Y₃ represents CH, CCH₃, CF, CCl, CCF₃, CCHF₂, CCH₂F, C(OCH₃), C(CN) or N.

In some embodiments of the present disclosure, wherein X₁ represents —CH₂—, —CF₂—, —CHF—, —O—, —S—, —CHOH—, —CH₂CH₂— or —C(CH₃)₂—.

In some embodiments of the present disclosure, wherein X₂ represents —CH₂—, —CF₂—, —CHF—, —O—, —S—, —CHOH—, —CH₂CH₂— or —C(CH₃)₂—.

In some embodiments of the present disclosure, wherein L₁ represents —CH₂—, —CHF—, —CF₂—, —O—, —NH— or —NCH₃—.

In some embodiments of the present disclosure, wherein L₂ represents —NHC(O)—, —C(O)NH—, —NHC(O)NH—, —NHC(O)O—, —CH₂NH—, —NHCH₂—, —CH₂O—, —OCH₂— or —NH—.

In some embodiments of the present disclosure, wherein R² represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, phenyl, 5-6 membered heteroaryl, —OR^{a}, oxo, hydroxy (C₁-C₆alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein W₅ and W₂ combine to form a ring fused to ring A having the following structure:

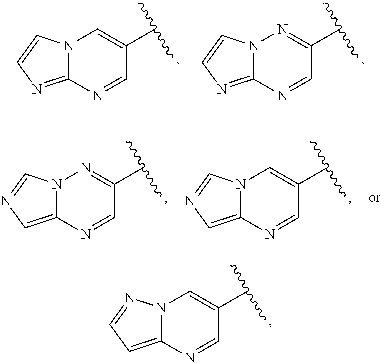 [SMILES: *c1cnc2nccn2c1 ; *c1cnc2nccn2n1 ; *c1cnc2cncn2n1 ; *c1cnc2cncn2c1 ; *c1cnc2ccnn2c1]

wherein the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}; and wherein the wavy line indicates the point of attachment with L₁.

In some embodiments of the present disclosure, wherein R² represents phenyl, pyridinyl, indazolyl, indolyl, thiazolyl, isothiazolyl, imidazolyl, pyrazolyl, oxazolyl, isoxazolyl, 1,2,4-oxadiazolyl, 1,3,4-oxadiazolyl, pyridazinyl, piperazinyl, pyrazinyl, cyclohexyl, benzothienyl, 1,2-benzoisoxazolyl, benzo[d] isothiazolyl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, phenyl, 5-6 membered heteroaryl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R² represents

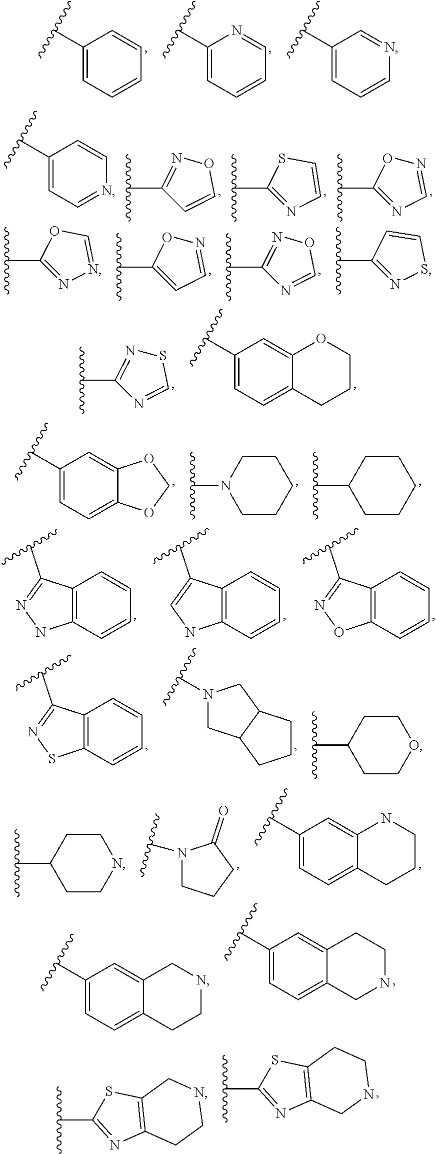

which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R² represents:

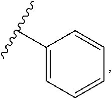 [SMILES: *c1ccccc1]

which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}, —CR^{a}R^{b}NR^{a}R^{b}, —CN, C₁-C₆ haloalkyl, or C₁-C₆ alkoxy.

In some embodiments of the present disclosure, wherein R³ represents hydrogen or CH₃.

The present disclosure provides a compound having the structure of formula (II) or a pharmaceutically acceptable salt, stereoisomers, isotopic isomers thereof,

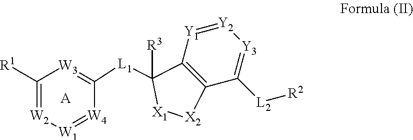

wherein, X₁ represents O, S, NR^{a}, (CR^{L}R^{L′})ₘ, C(O);
wherein, X₂ represents O, S, NR^{a}, (CR^{T}R^{T′})ₙ, C(O);
wherein, W₁ independently represents CR^{W1} or N;
wherein, W₂ independently represents CR^{W2} or N;
wherein, W₃ independently represents CR^{W3} or N;
wherein, W₄ independently represents CR^{W4} or N;
wherein, Y₁ independently represents CR^{Y1} or N;
wherein, Y₂ independently represents CR^{Y2} or N;
wherein, Y₃ independently represents CR^{Y3} or N;
wherein, each R^{W1}, R^{W2}, R^{W3}, R^{W4}, R^{Y1}, R^{Y2}, R^{Y3} independently represents hydrogen, deuterium, C₁-C₆ alkyl, deuterated C₁-C₆ alkyl, halogen, nitro, —OR^{a}, —SR^{a}, —P(O)R^{a}R^{b}, —CN, —S(O)₂R^{a}, —S(O)R^{a}, —SF₅, —NR^{a}R^{b}, —CR^{a}R^{b}NR^{a}R^{b}, —NR^{a}COR^{b}, —CONR^{a}R^{b}, C₁-C₆ haloalkyl, C₁-C₆ haloalkoxy, C₁-C₆ haloalkylthio, hydroxy (C₁-C₆ alkyl); or each R^{W1}, R^{W2}, R^{W3}, R^{W4}, R^{Y1}, R^{Y2}, R^{Y3} independently represents C₃-C₁₀ cycloalkyl, C₆-C₁₀cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
or W₁ and W₂ together with the atom which they are attached to form a 5-6 membered saturated or unsaturated ring fused to ring A, and the said ring may optionally contain 0, 1, 2 or 3 heteroatoms selected from O, N, or S;
or W₁ and W₄ together with the atom which they are attached to form a 5-6 membered saturated or unsaturated ring fused to ring A, and the said ring may optionally contain 0, 1, 2 or 3 heteroatoms selected from O, N, or S;
wherein, L₁ represents absent, —(CR^{a}R^{b})ₒ—, —CR^{a}R^{b}O—, —OCR^{a}R^{b}—, —O—, —S—, —S(O)₂—, —C(O)—, —NR^{a}—, —S(O)₂NR^{a}—, —OC(O)—, —C(O)O—, —NR^{a}S(O)₂—, —CR^{a}R^{b}NR^{a}—, or —NR^{a}CR^{a}R^{b}—;
wherein, L₂ represents absent, —(CR^{a}R^{b})ₒ—, —CR^{a}R^{b}O—, —OCR^{a}R^{b}—, —O—, —C(O)—, —NR^{a}—, —S(O)₂NR^{a}—, —OC(O)—, —C(O)O—, —NR^{a}S(O)₂—, —CR^{a}R^{b}NR^{a}—, —NR^{a}CR^{a}R^{b}—, —NR^{a}C(O)NR^{b}—, —NR^{a}C(O)NR^{b}CR^{a}R^{b}—, —NR^{a}C(O)O—, —NR^{a}C(O)—, or —C(O)NR^{a}—.

Wherein, R¹ represents hydrogen, deuterium, C₁-C₆ alkyl, deuterated C₁-C₆ alkyl, halogen, —OR^{a}, —SR^{a}, —P(O)R^{a}R^{b}, —CN, —S(O)₂R^{a}, —S(O)R^{a}, —SF₅, —NR^{a}R^{b}, C₁-C₆ haloalkyl, C₁-C₆ haloalkoxy, C₁-C₆ haloalkylthio, hydroxy (C₁-C₆ alkyl); or R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl, which are substitute C₁-C₆ haloalkyld by 0-4 substituents that selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}; and when R¹ is a ring, any two adjacent substituents on the said ring may combine to form a ring, and the said ring may optionally contain 0, 1 or 2 heteroatoms selected from O, N, or S; and further, the said ring may optionally be substituted by halogen, hydroxy or C₁-C₆ alkyl;
or R¹ and W₂ together with the atom which they are attached to form a 5-6 membered saturated or unsaturated ring fused to ring A, and the said ring may optionally contain 0, 1, 2 or 3 heteroatoms selected from O, N, or S; and further, the said ring may optionally substituted by 0, 1 or 2 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}—
wherein, R² represents C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, —CR^{a}R^{b}C₆-C₁₀ aryl, —CR^{a}R^{b} (5-10 membered heteroaryl); which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, C₆—C to aryl, 5-10 membered heteroaryl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, C₃-C₆ halocycloalkyl, C₃-C₆ haloheterocycloalkyl, C₁-C₆ alkyl-substituted C₃-C₆ heterocycloalkyl, —SO₃R^{a}, —SR^{a}, —S(O)₂R^{a}, —S(O)R^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}; and when R² is a ring, any two adjacent substituents on the said ring may combine to form a ring, and the said ring may optionally contain 0, 1 or 2 heteroatoms selected from O, N, or S; and further, the said ring may optionally be substituted by halogen, hydroxy or C₁-C₆ alkyl;
wherein, R³ represents hydrogen, halogen, C₁-C₆ haloalkyl, C₁-C₆ alkyl, or hydroxy;
or R³ and W₃ together with the atom which they are attached to form a 5-7 membered saturated or unsaturated ring fused to ring A, and the said ring may optionally contain 0, 1 or 2 heteroatoms selected from O, N, or S; and further, the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, each R^{a} and R^{b} independently represents hydrogen, deuterium, halogen, C₁-C₆ alkyl, C₁-C₆ haloalkyl, hydroxy (C₁-C₆ alkyl), (CH₃)₂N—(C₁-C₆ alkyl), C₃-C₁₀ cycloalkyl; or R^{a} and R^{b} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, each R^{L} and R^{L′} independently represents hydrogen, deuterium, halogen, C₁-C₆ alkyl, C₁-C₆ haloalkyl, hydroxy (C₁-C₆ alkyl), C₃-C₁₀ cycloalkyl; or R^{L} and R^{L′} together with the carbon atom which they are attached to form a 3-6 membered ring, and the said ring may optionally contain 0, 1 or 2 heteroatoms selected from O, N, or S; and further, the said ring may be optionally substituted by halogen, hydroxy or C₁-C₆ alkyl;
wherein, each R^{T} and R^{T′} independently represents hydrogen, deuterium, halogen, C₁-C₆ alkyl, C₁-C₆ haloalkyl, hydroxy (C₁-C₆ alkyl), C₃-C₁₀ cycloalkyl; or R^{T} and R^{T′} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, o, m, n represents the integer of 1, 2 or 3.

In some embodiments of the present disclosure, the compound has a structure as shown in formula (II):

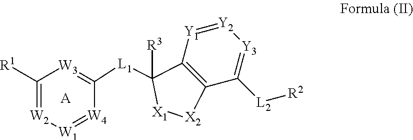

wherein, X₁ represents O, S, NR^{a}, (CR^{L}R^{L′})ₘ, C(O);
wherein, X₂ represents O, S, NR^{a}, (CR^{T}R^{T′})ₙ, C(O);
wherein, W₁ independently represents CR^{W1} or N;
wherein, W₂ independently represents CR^{W2} or N;
wherein, W₃ independently represents CR^{W3} or N;
wherein, W₄ independently represents CR^{W4} or N;
wherein, Y₁ independently represents CR^{Y1} or N;
wherein, Y₂ independently represents CR^{Y2} or N;
wherein, Y₃ independently represents CR^{Y3} or N;
wherein, each R^{W1}, R^{W2}, R^{W3}, R^{W4}, R^{Y1}, R^{Y2}, R^{Y3} independently represents hydrogen, deuterium, C₁-C₆ alkyl, deuterated C₁-C₆ alkyl, halogen, nitro, —OR^{a}, —SR^{a}, —P(O)R^{a}R^{b}, —CN, —S(O)₂R^{a}, —S(O)R^{a}, —SF₅, —NR^{a}R^{b}, —CR^{a}R^{b}NR^{a}R^{b}, —NR^{a}COR^{b}, —CONR^{a}R^{b}, C₁-C₆ haloalkyl, C₁-C₆ haloalkoxy, C₁-C₆ haloalkylthio, hydroxy (C₁-C₆alkyl); or each R^{W1}, R^{W2}, R^{W3}, R^{W4}, R^{Y1}, R^{Y2}, R^{Y3} independently represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
or W₁ and W₂ together with the atom which they are attached to form a 5-6 membered saturated or unsaturated ring fused to ring A, and the said ring may optionally contain 0, 1, 2 or 3 heteroatoms selected from O, N, or S;
or W₁ and W₄ together with the atom which they are attached to form a 5-6 membered saturated or unsaturated ring fused to ring A, and the said ring may optionally contain 0, 1, 2 or 3 heteroatoms selected from O, N, or S;
wherein, L₁ represents absent, —(CR^{a}R^{b})ₒ—, —CR^{a}R^{b}O—, —OCR^{a}R^{b}—, —O—, —S—, —S(O)₂—, —C(O)—, —NR^{a}—, —S(O)₂NR^{a}—, —OC(O)—, —C(O)O—, —NR^{a}S(O)₂—, —CR^{a}R^{b}NR^{a}—, or —NR^{a}CR^{a}R^{b}—;
wherein, L₂ represents absent, —(CR^{a}R^{b})ₒ—, —CR^{a}R^{b}O—, —OCR^{a}R^{b}—, —O—, —C(O)—, —NR^{a}—, —S(O)₂NR^{a}—, —OC(O)—, —C(O)O—, —NR^{a}S(O)₂—, —CR^{a}R^{b}NR^{a}—, —NR^{a}CR^{a}R^{b}—, —NR^{a}C(O)NR^{b}—, —NR^{a}C(O)NR^{b}CR^{a}R^{b}—, —NR^{a}C(O)O—, —NR^{a}C(O)—, or —C(O)NR^{a}—.

Wherein, R¹ represents hydrogen, deuterium, C₁-C₆ alkyl, deuterated C₁-C₆ alkyl, halogen, —OR^{a}, —SR^{a}, —P(O)R^{a}R^{b}, —CN, —S(O)₂R^{a}, —S(O)R^{a}, —SF₅, —NR^{a}R^{b}, C₁-C₆ haloalkyl, C₁-C₆ haloalkoxy, C₁-C₆ haloalkylthio, hydroxy (C₁-C₆ alkyl); or R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl, which are substituted by 0-4 substituents that selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR⁸, —SF₅, —C(O)R^{a}, —C(O)OR^{a}, —OC(O)R^{a}, —OC(O)NR^{a}R^{b}, —NR^{a}COR^{b} or —CONR^{a}R^{b};
or R¹ and W₂ together with the atom which they are attached to form a 5-6 membered saturated or unsaturated ring fused to ring A, and the said ring may optionally contain 0, 1 or 2 heteroatoms selected from O, N, or S; and further, the said ring may optionally substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, R² represents C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl; which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CN, C₁-C₆ haloalkyl, C₁-C₆ haloalkoxy, —SO₃R^{a}, —SR^{a}, —S(O)₂R^{a}, —S(O)R^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, R³ represents hydrogen, halogen, C₁-C₆ haloalkyl, C₁-C₆ alkyl, or hydroxy;
or R³ and W₃ together with the atom which they are attached to form a 5-7 membered saturated or unsaturated ring fused to ring A. and the said ring may optionally contain 0, 1 or 2 heteroatoms selected from O, N, or S; and further, the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, each R^{a} and R^{b} independently represents hydrogen, C₁-C₆ alkyl, C₁-C₆ haloalkyl, hydroxy (C₁-C₆ alkyl), C₃-C₁₀ cycloalkyl; or R^{a} and R^{b} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, each R^{L} and R^{L′} independently represents hydrogen, C₁-C₆ alkyl, C₁-C₆ haloalkyl, hydroxy (C₁-C₆ alkyl), C₃-C₁₀ cycloalkyl; or R^{L} and R^{L′} together with the carbon atom which they are attached to form a 3-6 membered ring, and the said ring may optionally contain 0, 1 or 2 heteroatoms selected from O, N, or S; and further, the said ring may be optionally substituted by halogen, hydroxy or C₁-C₆ alkyl;
wherein, each R^{T} and R^{T′} independently represents hydrogen, C₁-C₆ alkyl, C₁-C₆ haloalkyl, hydroxy (C₁-C₆ alkyl), C₃-C₁₀ cycloalkyl; or R^{T} and R^{T′} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, o, m, n represents the integer of 1, 2 or 3.

In some embodiments of the present disclosure, wherein W₁ represents CH, CCH₃, CF, CCF₃, CCHF₂, CHF₂, COCH₃, C(CN) or N.

In some embodiments of the present disclosure, wherein W₁ represents N.

In some embodiments of the present disclosure, wherein W₂ represents CH, CCH₃, CF, CCF₃, CCHF₂, CHF₂, COCH₃, CNH₂, CNHCH₃, CN(CH₃)₂, CH₂NCH₃, CH₂N(CH₃)₂, C(CN) or N.

In some embodiments of the present disclosure, wherein W₃ represents CH, CCH₃, CF, CCF₃, CCHF₂, CHF₂, COCH₃, C(CN) or N.

In some embodiments of the present disclosure, wherein W₄ represents CH, CCH₃, CF, CCF₃, CCHF₂, CHF₂, COCH₃, CNH₂, CNHCH₃, C(CN) or N.

In some embodiments of the present disclosure, wherein Y₁ represents CH, CCH₃, CF, CCl, CCF₃, CCHF₂, CHF₂, COCH₃, C(CN) or N.

In some embodiments of the present disclosure, wherein Y₂ represents CH, CCH₃, CF, CCl, CCF₃, CCHF₂, CHF₂, COCH₃, C(CN) or N.

In some embodiments of the present disclosure, wherein Y₃ represents CH, CCH₃, CF, CCl, CCF₃, CCHF₂, CHF₂, COCH₃, C(CN) or N.

In some embodiments of the present disclosure, wherein X₁ represents CH₂, CF₂, CHF, O, S, CHOH, CH₂CH₂ or C(CH₃)₂.

In some embodiments of the present disclosure, wherein X₂ represents CH₂, CF₂, CHF, O, S, CHOH, CH₂CH₂ or C(CH₃)₂.

In some embodiments of the present disclosure, wherein L₁ represents CH₂, CF₂, O, NH or NCH₃.

In some embodiments of the present disclosure, wherein L₂ represents —NHC(O)—, —C(O)NH—, —NHC(O)NH—, —NHC(O)O—, —CH₂NH—, —NHCH₂—, —CH₂O—, —OCH₂— or —NH—.

In some embodiments of the present disclosure, wherein R¹ represents C₁-C₆ alkyl, halogen, —OH, O(C₁-C₆ alkyl); or R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynvl, C₃-C₆ cycloalkyl, —OR^{a}, oxo, hydroxy (C₁-C₆ alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, —OR^{a}, hydroxy (C₁-C₆ alkyl), —CN, C₁-C₆ haloalkyl.

In some embodiments of the present disclosure, wherein R¹ represents 5-10 membered heteroaryls, which are substituted by 0-4 substituents selected from deuterium, halogen, C₁-C₆ alkyl, —OR^{a}, hydroxy (C₁-C₆ alkyl), —CN, C₁-C₆ haloalkyl.

In some embodiments of the present disclosure, wherein R¹ represents pyrazolyl, triazolyl, oxazolyl, thiazolyl, imidazolyl, which are substituted by 0-4 substituents selected from deuterium, halogen, C₁-C₆ alkyl, —OR^{a}, hydroxy (C₁-C₆ alkyl), —CN, C₁-C₆ haloalkyl.

In some embodiments of the present disclosure, wherein R¹ represents

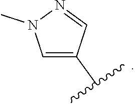 [SMILES: *c1cnn(C)c1]

In some embodiments of the present disclosure, wherein R² represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryls, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein R¹ and W₂ combine to form a ring fused to ring A having the following structure:

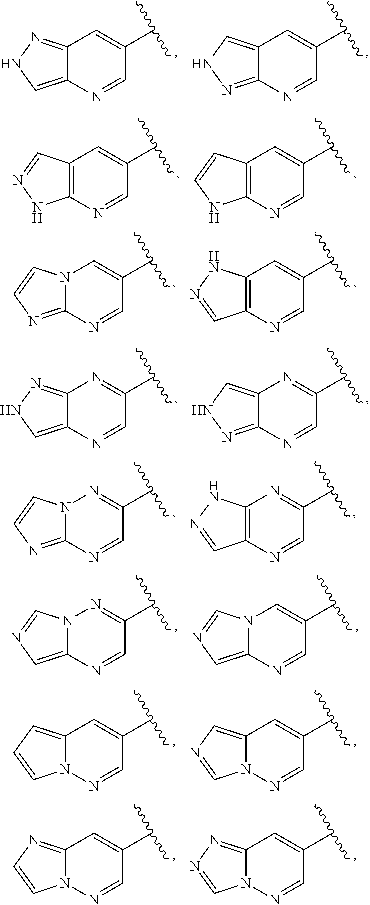

wherein the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}; and wherein the wavy line indicates the point of attachment with L₁.

In some embodiments of the present disclosure, wherein R¹ and W₂ combine to form a ring fused to ring A having the following structure:

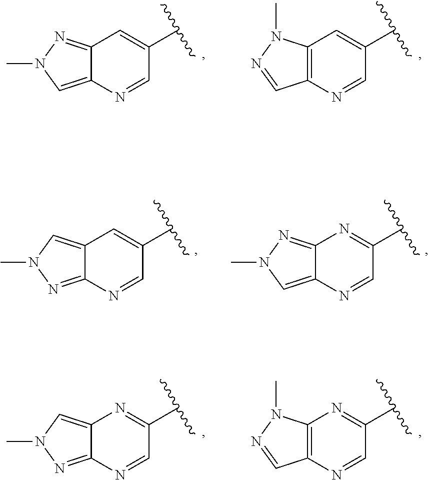 [SMILES: *c1cnc2cn(C)nc2c1 ; *c1cnc2cnn(C)c2c1 ; *c1cnc2nn(C)cc2c1 ; *c1cnc2cn(C)nc2n1 ; *c1cnc2nn(C)cc2n1 ; *c1cnc2cnn(C)c2n1]

wherein the wavy line indicates the point of attachment with L₁.

In some embodiments of the present disclosure, wherein R² represents phenyl, pyridinyl, indazolyl, indolyl, thiazolyl, isothiazolyl, imidazolyl, pyrazolyl, oxazolyl, isoxazolyl, 1,2,4-oxadiazolyl, 1,3,4-oxadiazolyl, pyridazinyl, piperazinyl, pyrazinyl, cyclohexyl, benzothienyl, 1,2-benzoisoxazolyl, benzo[d] isothiazolyl, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R² represents:

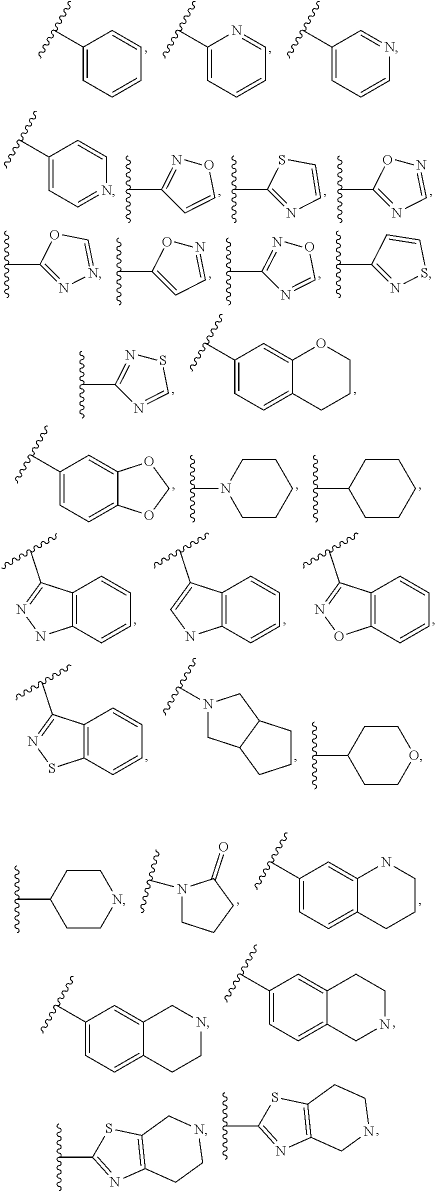

which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R² represents:

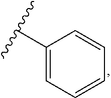 [SMILES: *c1ccccc1]

which is substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R³ represents hydrogen or CH₃.

Specifically, the present disclosure provides compounds having the following structures:

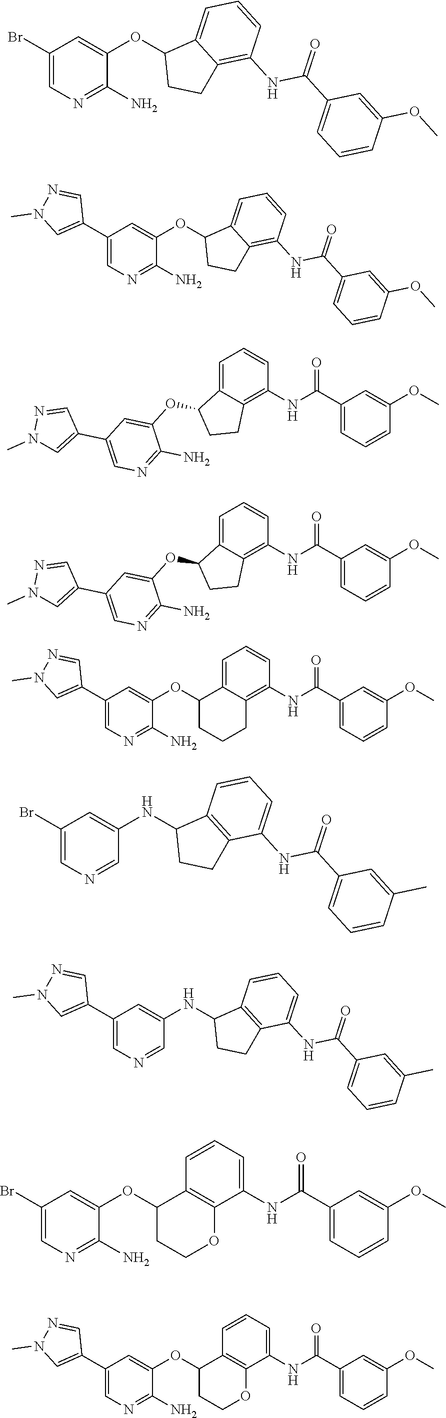

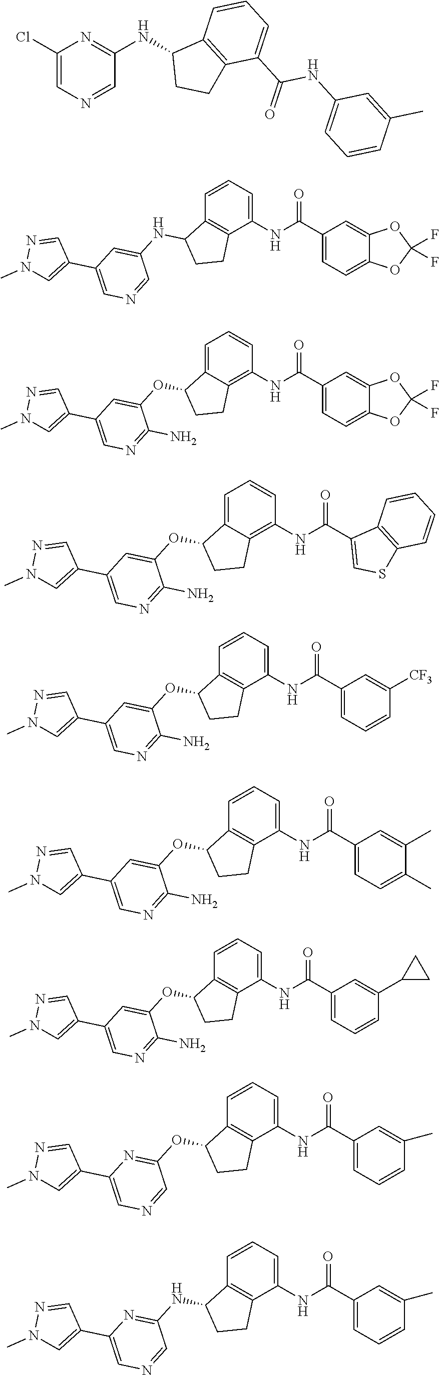

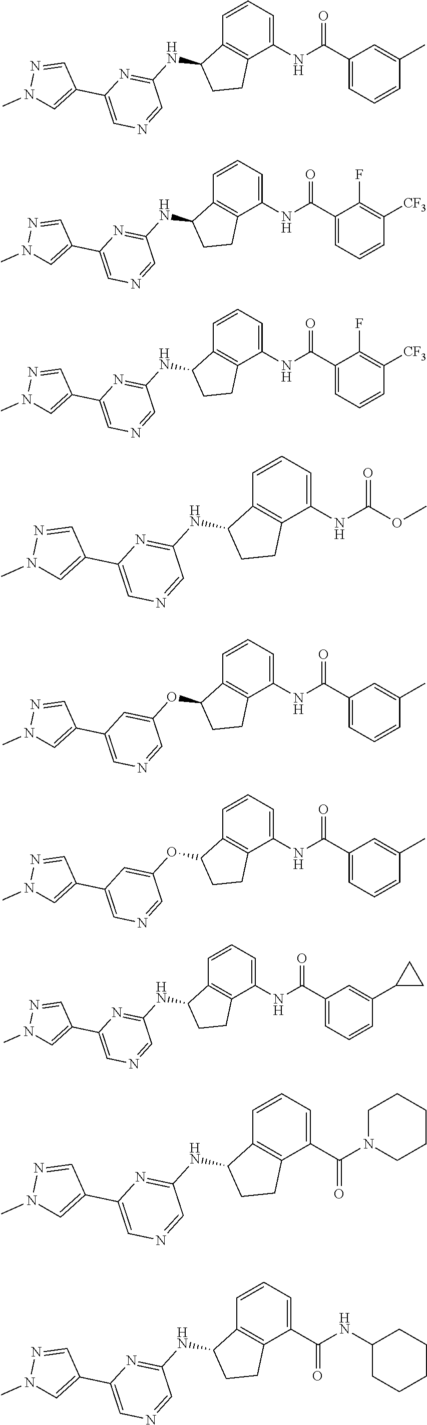

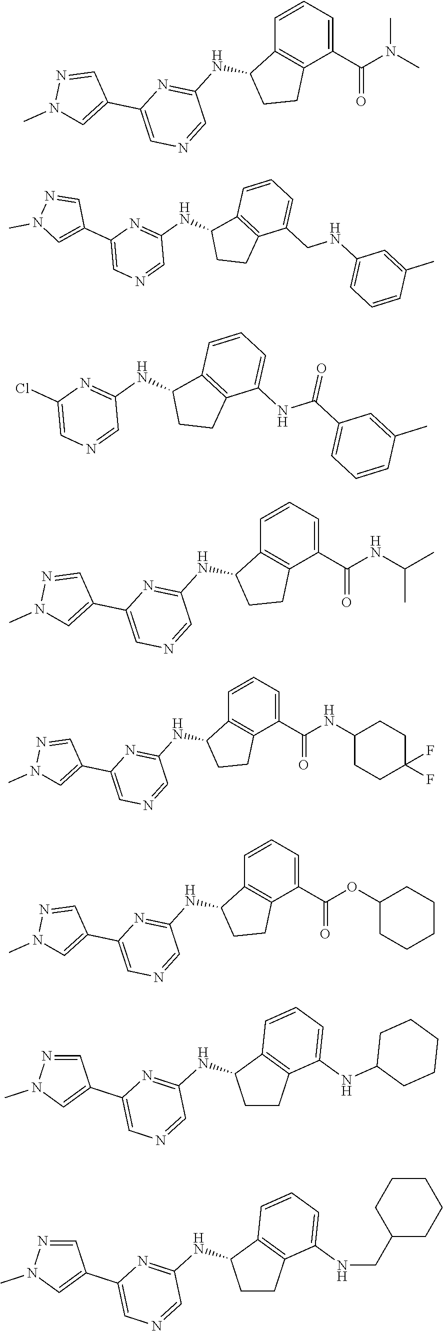

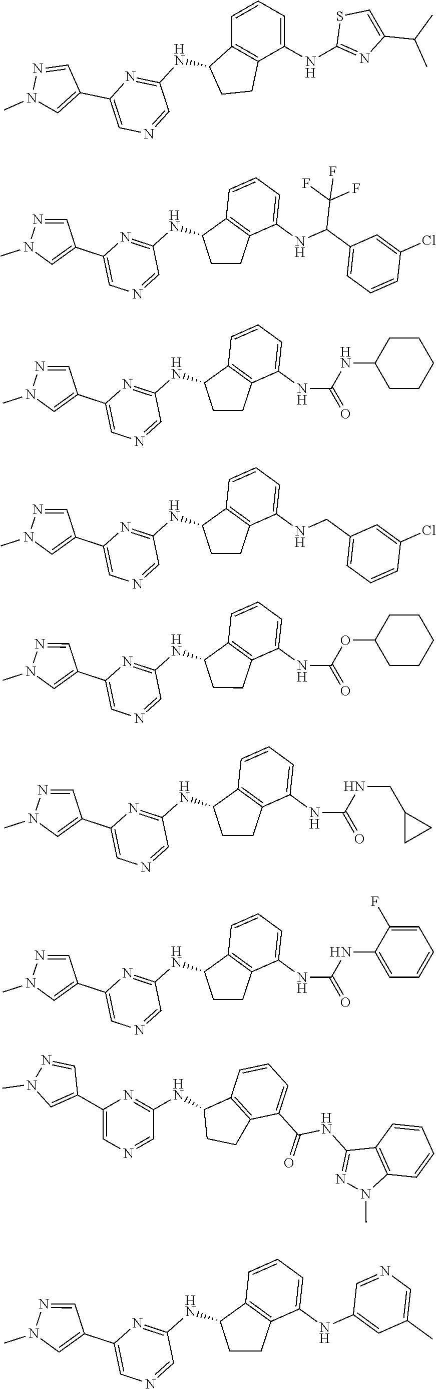

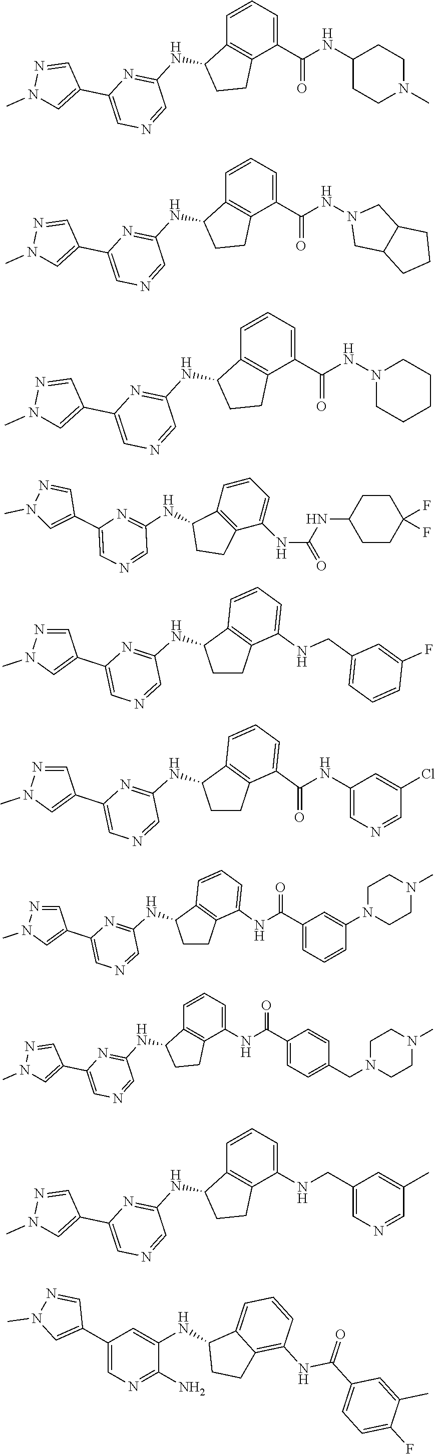

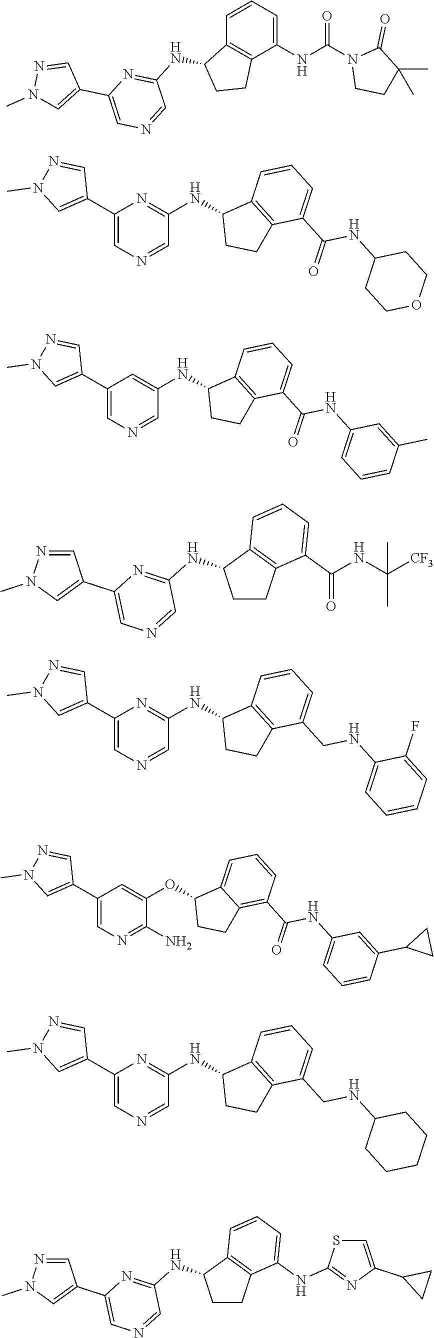

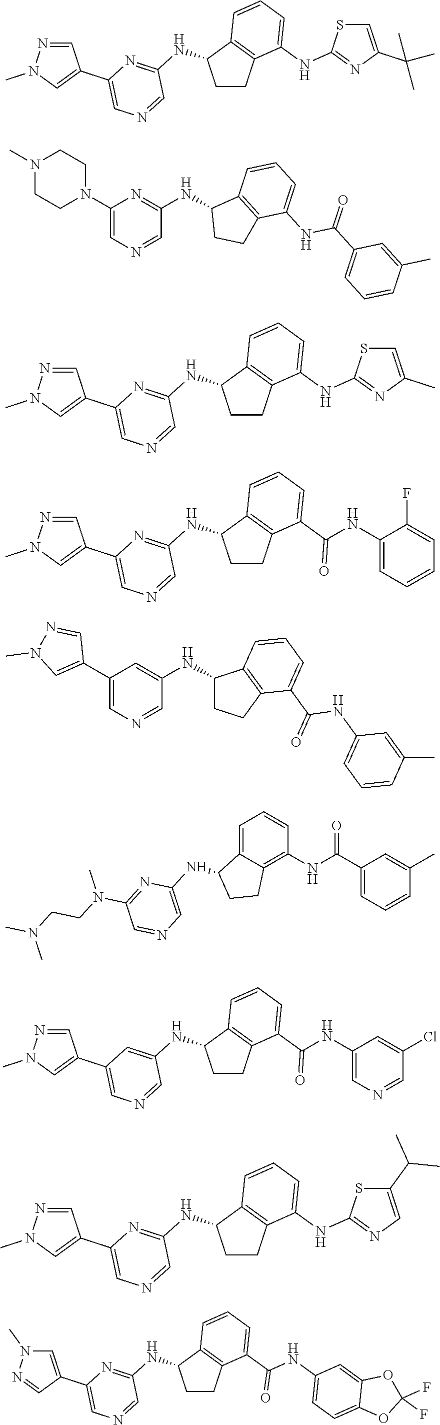

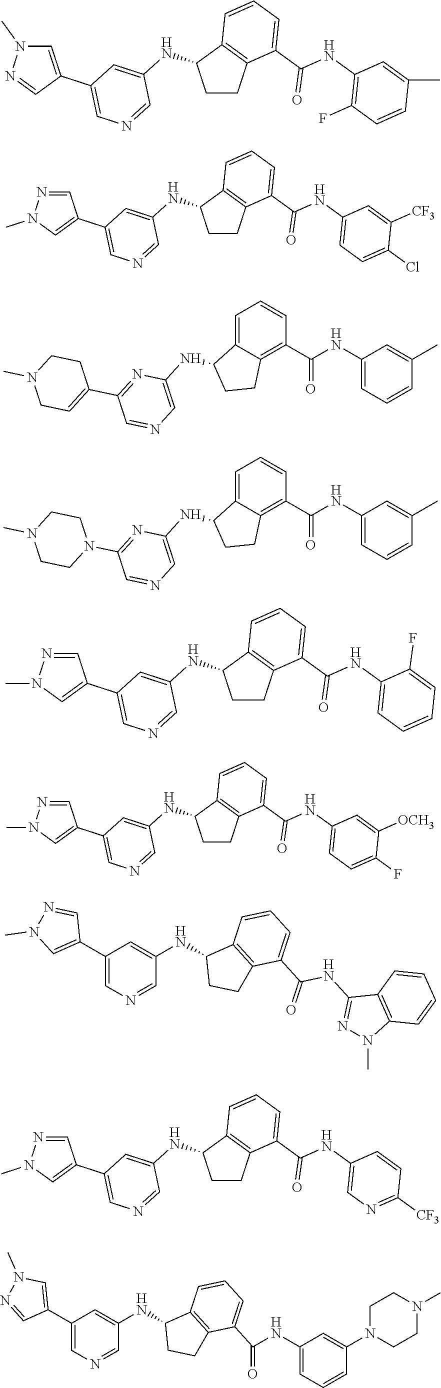

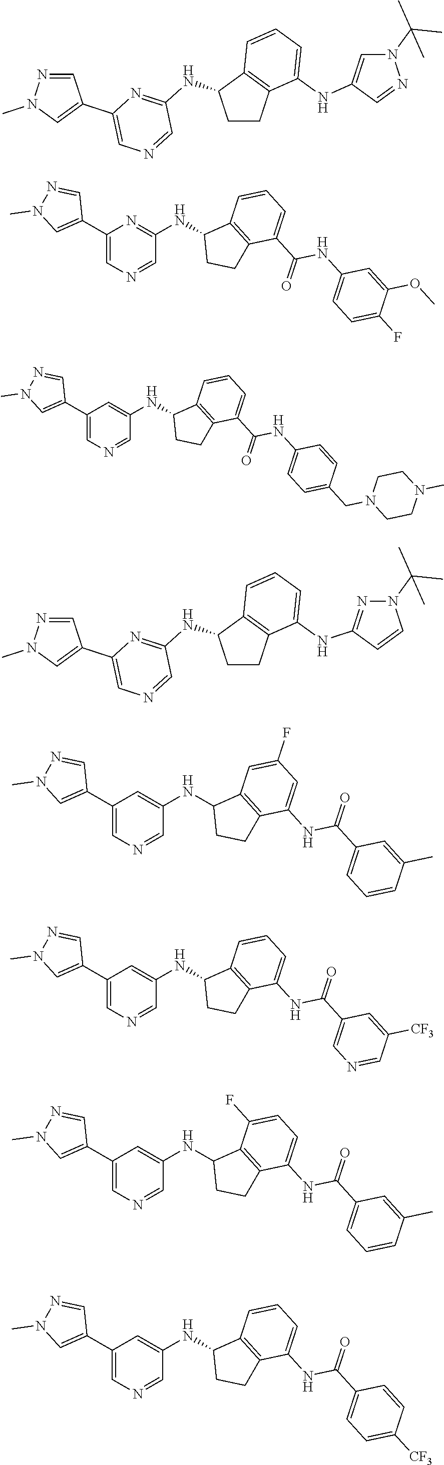

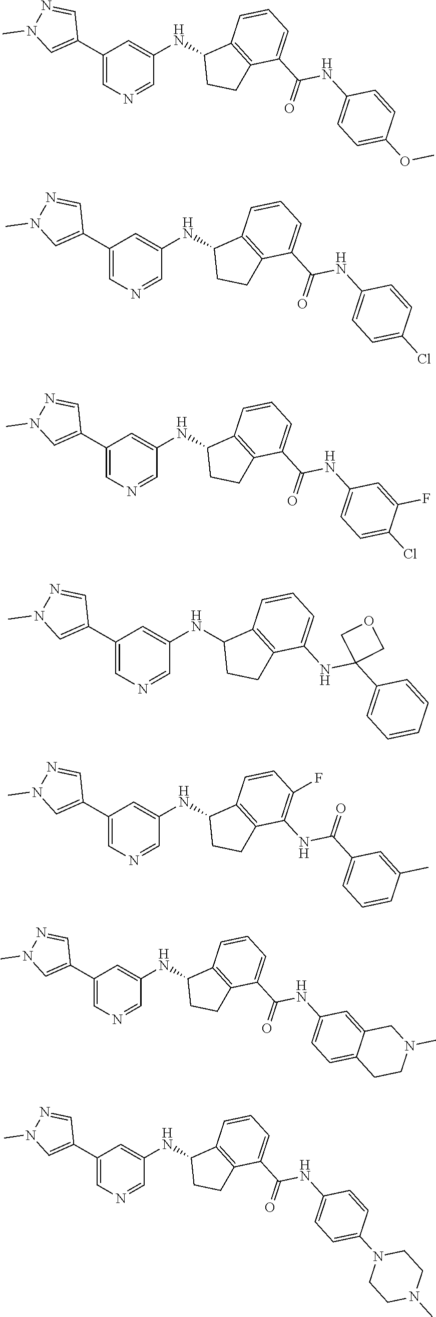

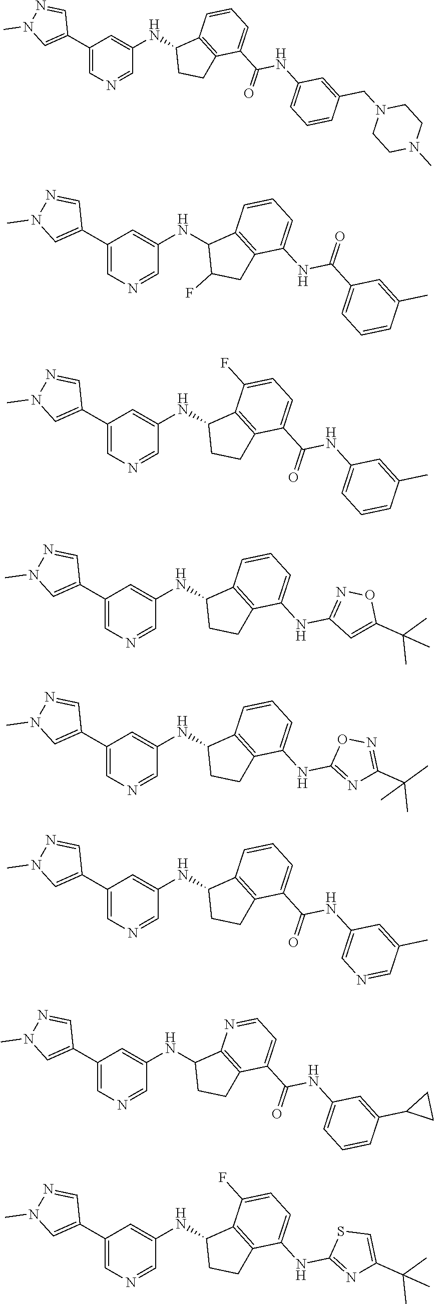

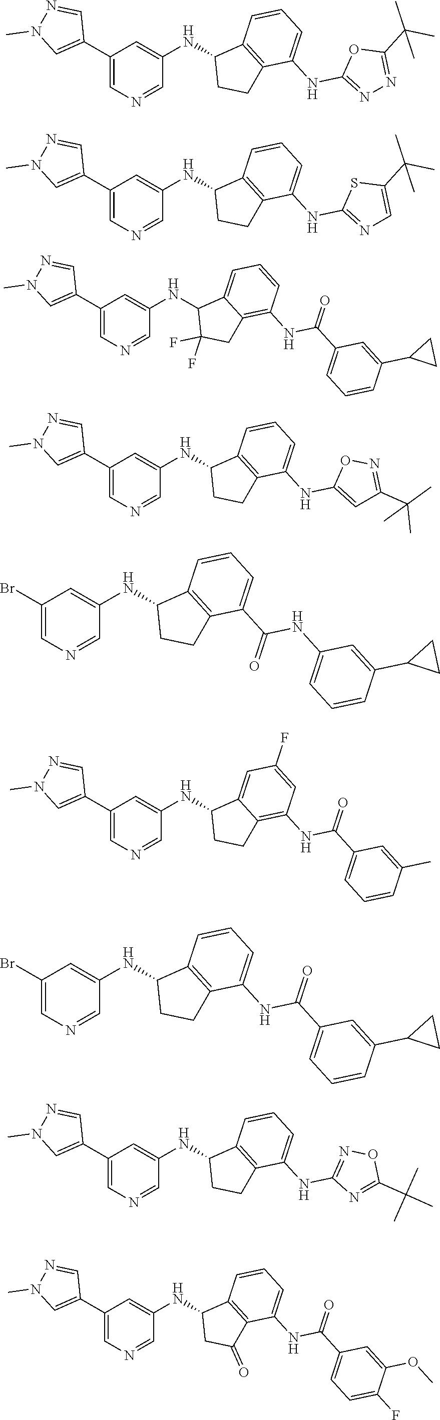

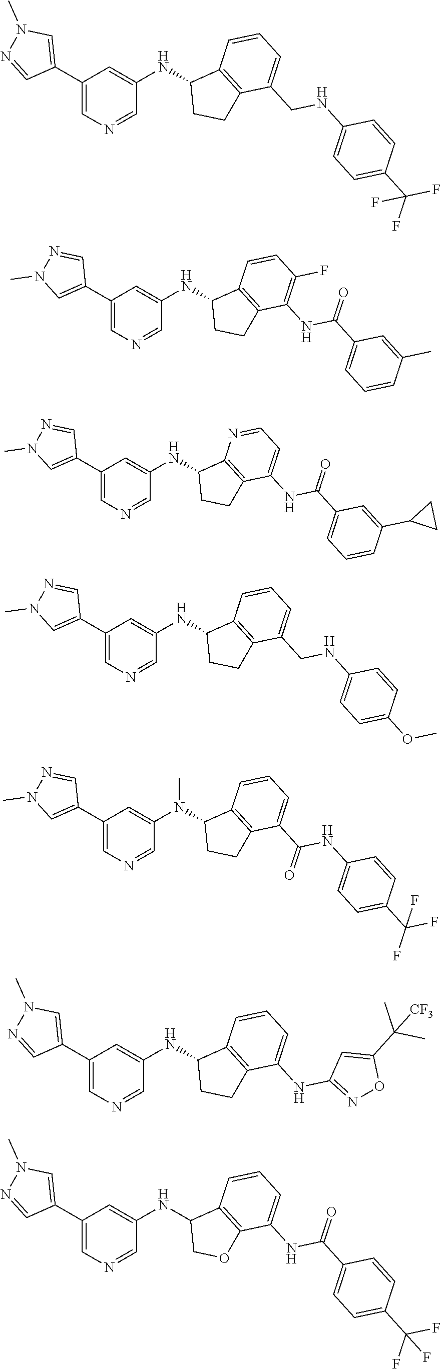

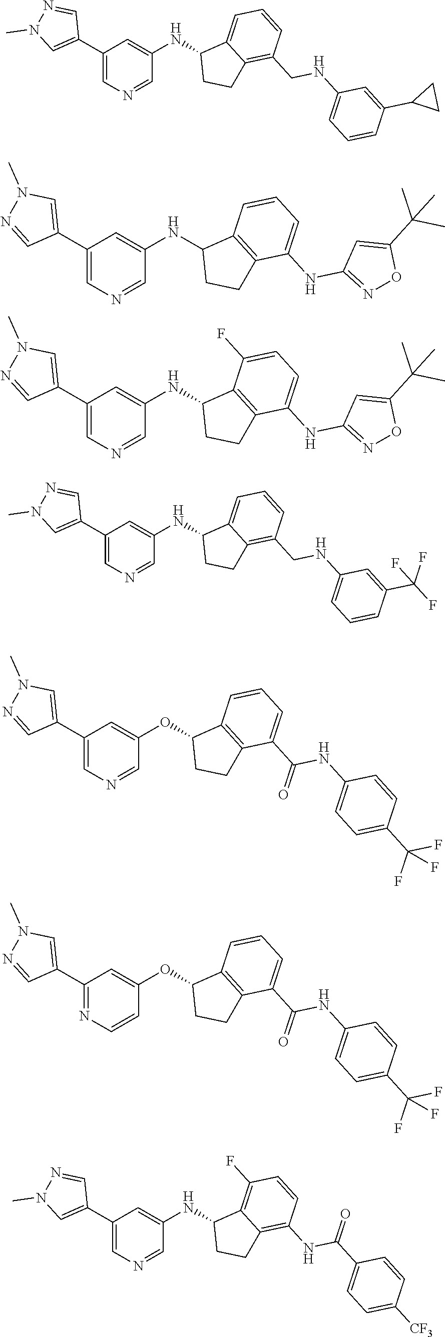

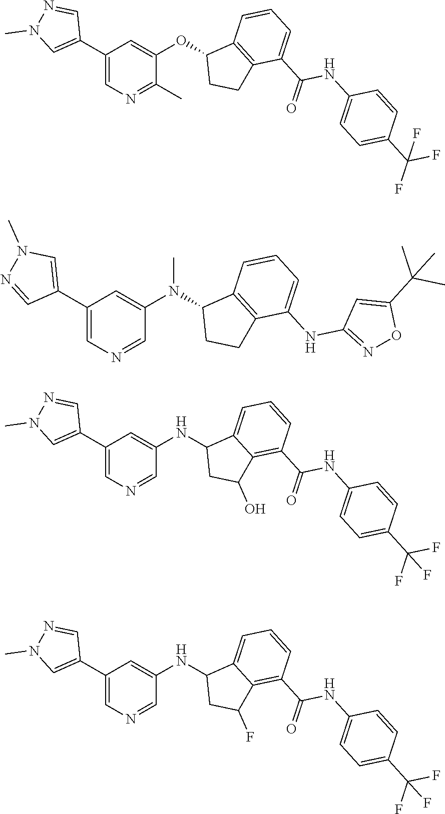

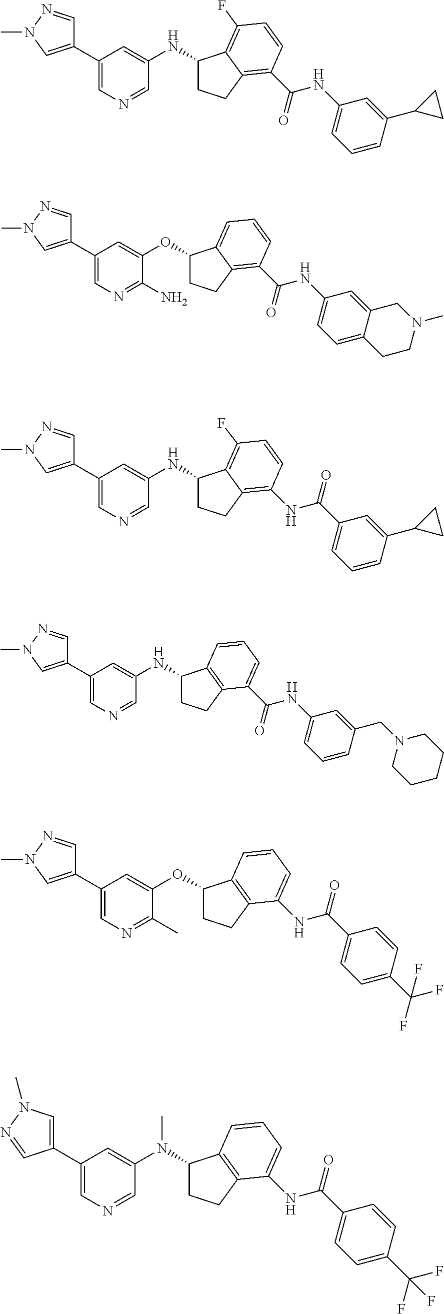

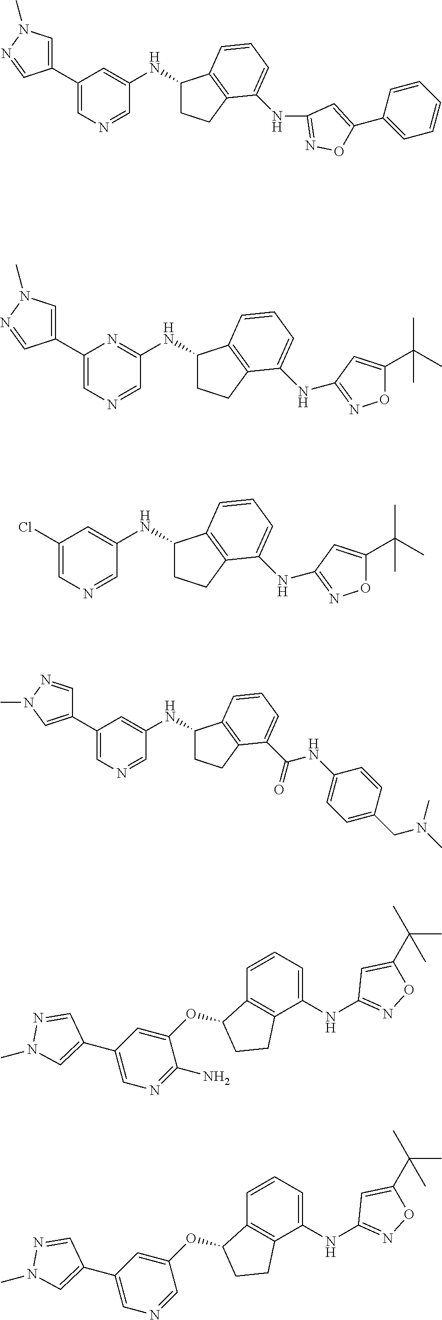

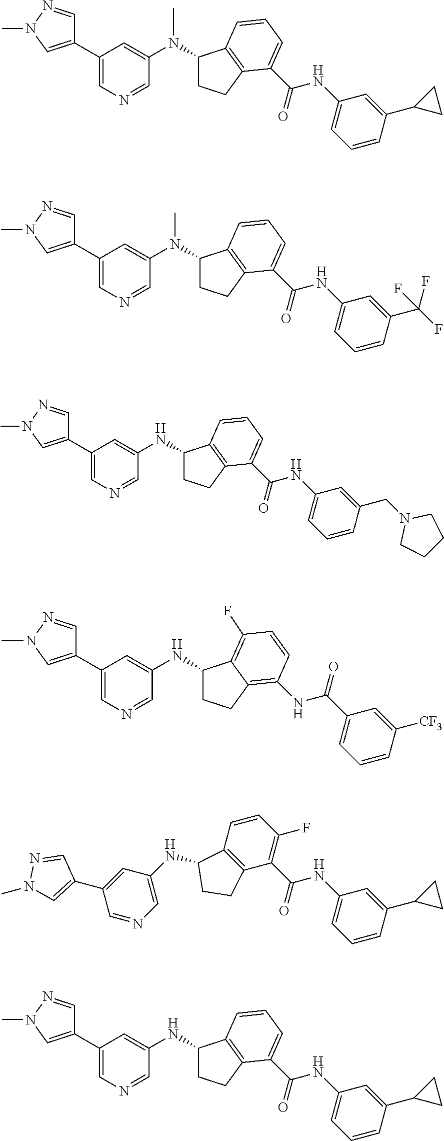 [SMILES: CN(c1cncc(-c2cnn(C)c2)c1)[C@H]1CCc2c(C(=O)Nc3cccc(C4CC4)c3)cccc21 ; CN(c1cncc(-c2cnn(C)c2)c1)[C@H]1CCc2c(C(=O)Nc3cccc(C(F)(F)F)c3)cccc21 ; Cn1cc(-c2cncc(N[C@H]3CCc4c(C(=O)Nc5cccc(CN6CCCC6)c5)cccc43)c2)cn1 ; Cn1cc(-c2cncc(N[C@H]3CCc4c(NC(=O)c5cccc(C(F)(F)F)c5)ccc(F)c43)c2)cn1 ; Cn1cc(-c2cncc(N[C@H]3CCc4c3ccc(F)c4C(=O)Nc3cccc(C4CC4)c3)c2)cn1 ; Cn1cc(-c2cncc(N[C@H]3CCc4c(C(=O)Nc5cccc(C6CC6)c5)cccc43)c2)cn1]

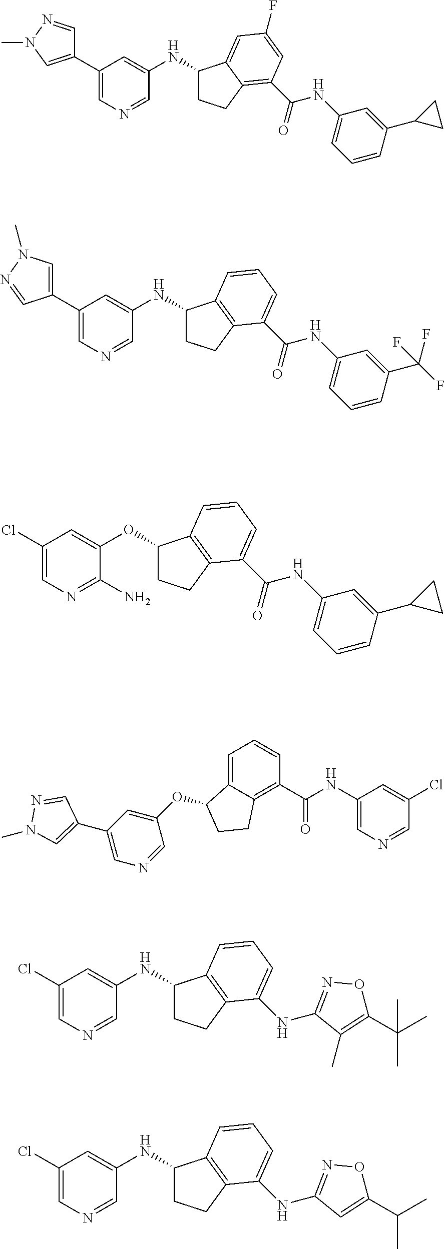 [SMILES: Cn1cc(-c2cncc(N[C@H]3CCc4c(C(=O)Nc5cccc(C6CC6)c5)cc(F)cc43)c2)cn1 ; Cn1cc(-c2cncc(N[C@H]3CCc4c(C(=O)Nc5cccc(C(F)(F)F)c5)cccc43)c2)cn1 ; Nc1ncc(Cl)cc1O[C@H]1CCc2c(C(=O)Nc3cccc(C4CC4)c3)cccc21 ; Cn1cc(-c2cncc(O[C@H]3CCc4c(C(=O)Nc5cncc(Cl)c5)cccc43)c2)cn1 ; Cc1c(Nc2cccc3c2CC[C@@H]3Nc2cncc(Cl)c2)noc1C(C)(C)C ; CC(C)c1cc(Nc2cccc3c2CC[C@@H]3Nc2cncc(Cl)c2)no1]

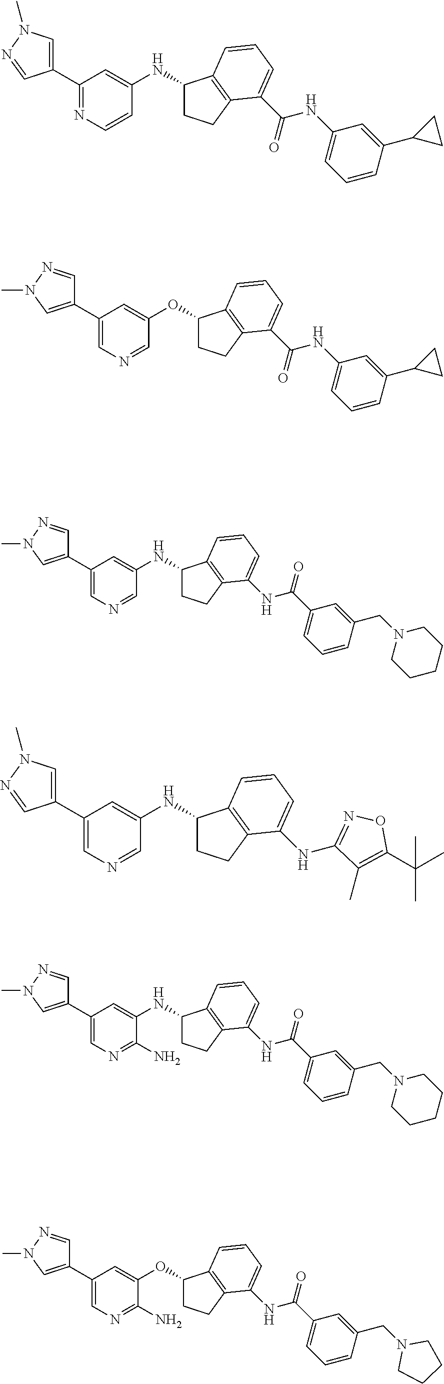

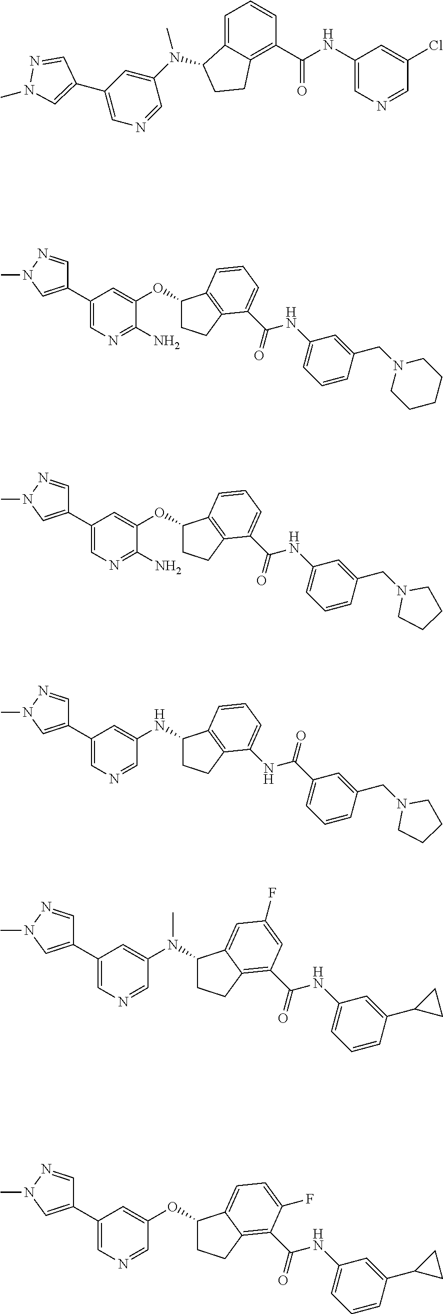 [SMILES: CN(c1cncc(-c2cnn(C)c2)c1)[C@H]1CCc2c(C(=O)Nc3cncc(Cl)c3)cccc21 ; Cn1cc(-c2cnc(N)c(O[C@H]3CCc4c(C(=O)Nc5cccc(CN6CCCCC6)c5)cccc43)c2)cn1 ; Cn1cc(-c2cnc(N)c(O[C@H]3CCc4c(C(=O)Nc5cccc(CN6CCCC6)c5)cccc43)c2)cn1 ; Cn1cc(-c2cncc(N[C@H]3CCc4c(NC(=O)c5cccc(CN6CCCC6)c5)cccc43)c2)cn1 ; CN(c1cncc(-c2cnn(C)c2)c1)[C@H]1CCc2c(C(=O)Nc3cccc(C4CC4)c3)cc(F)cc21 ; Cn1cc(-c2cncc(O[C@H]3CCc4c3ccc(F)c4C(=O)Nc3cccc(C4CC4)c3)c2)cn1]

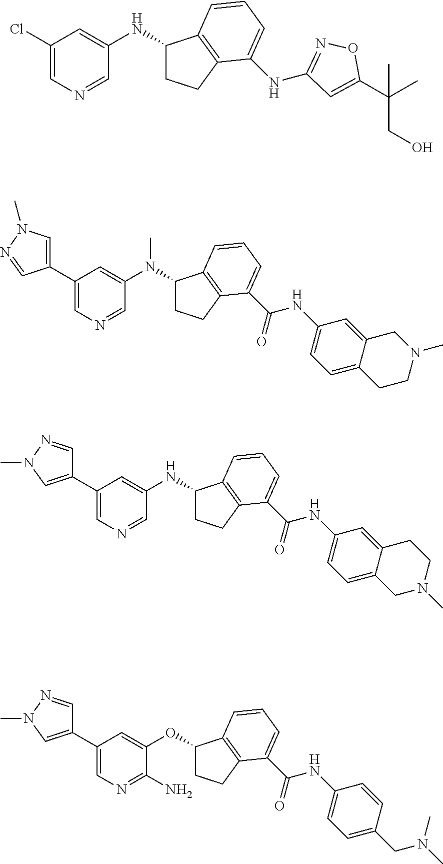

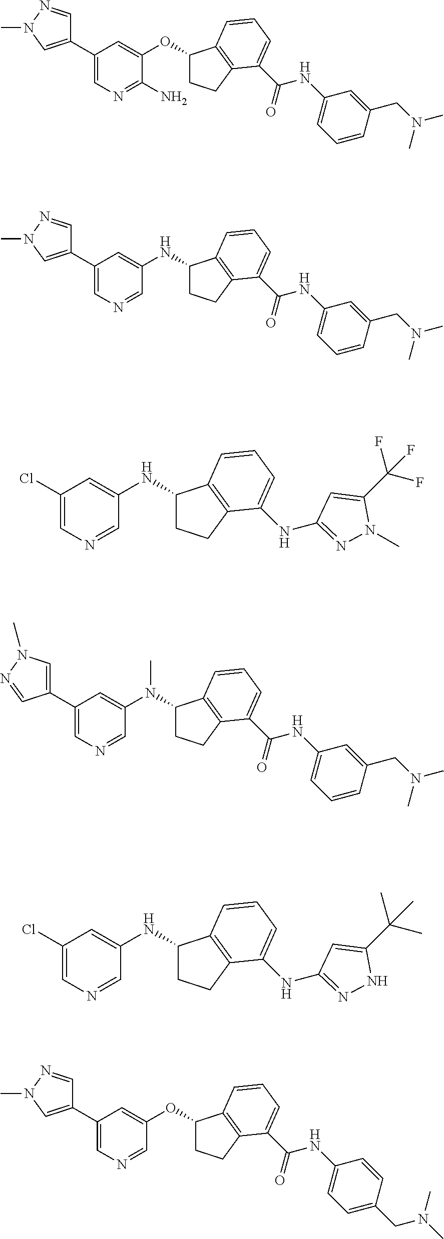

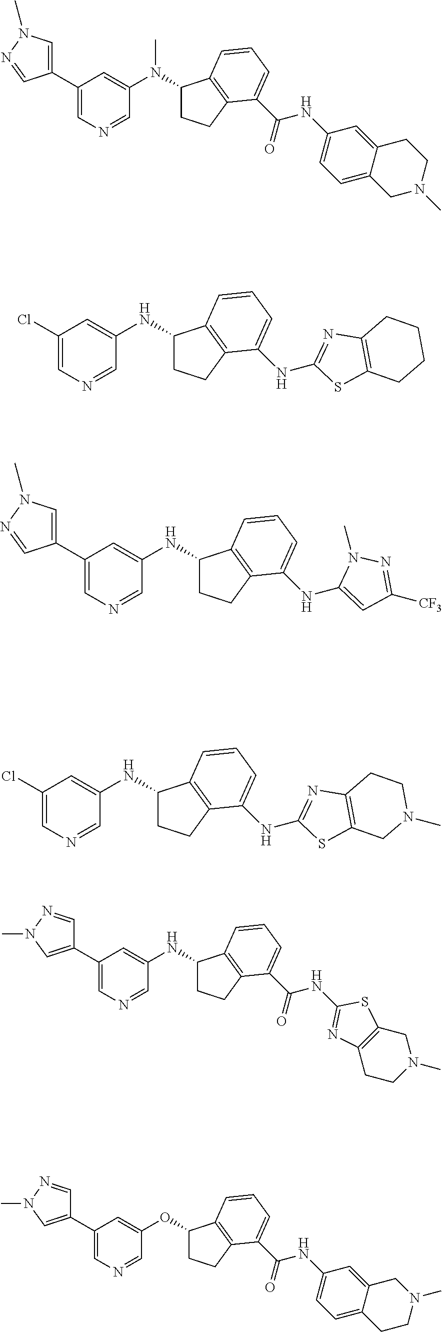

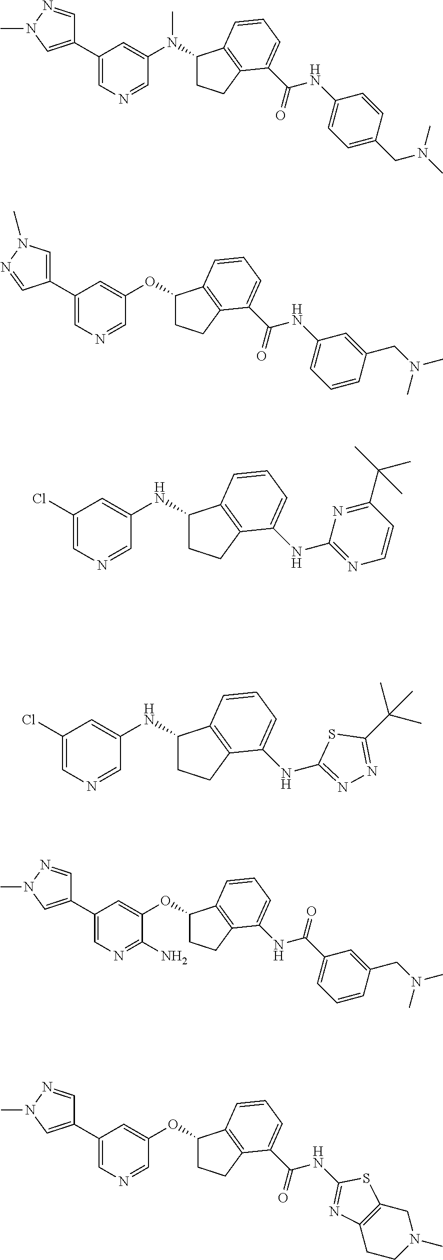

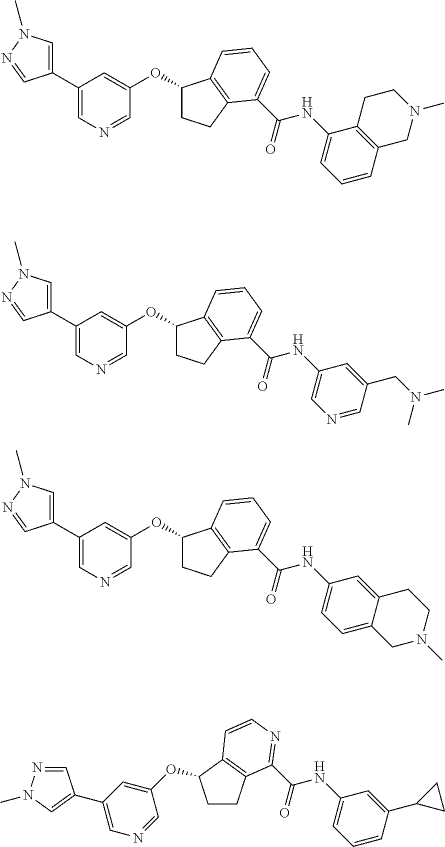

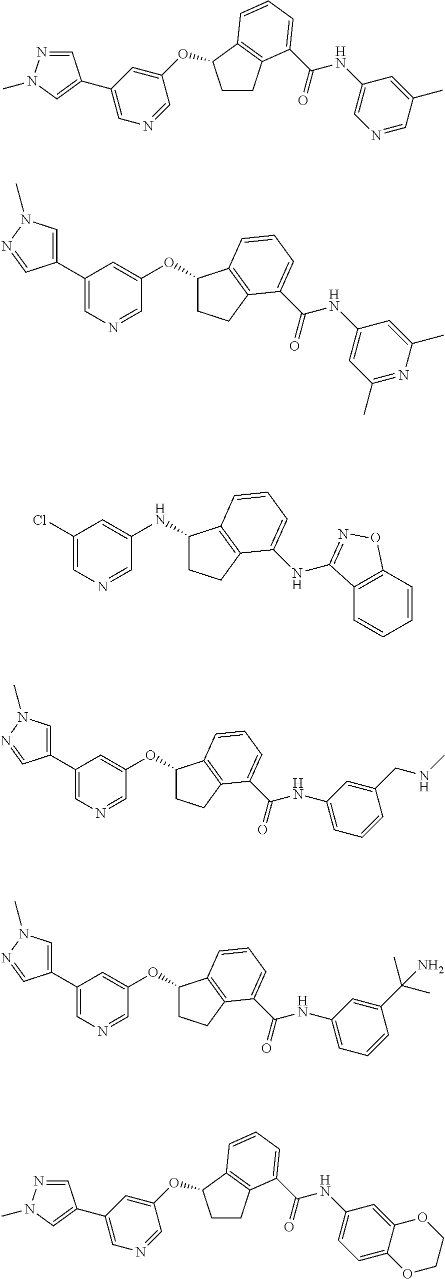

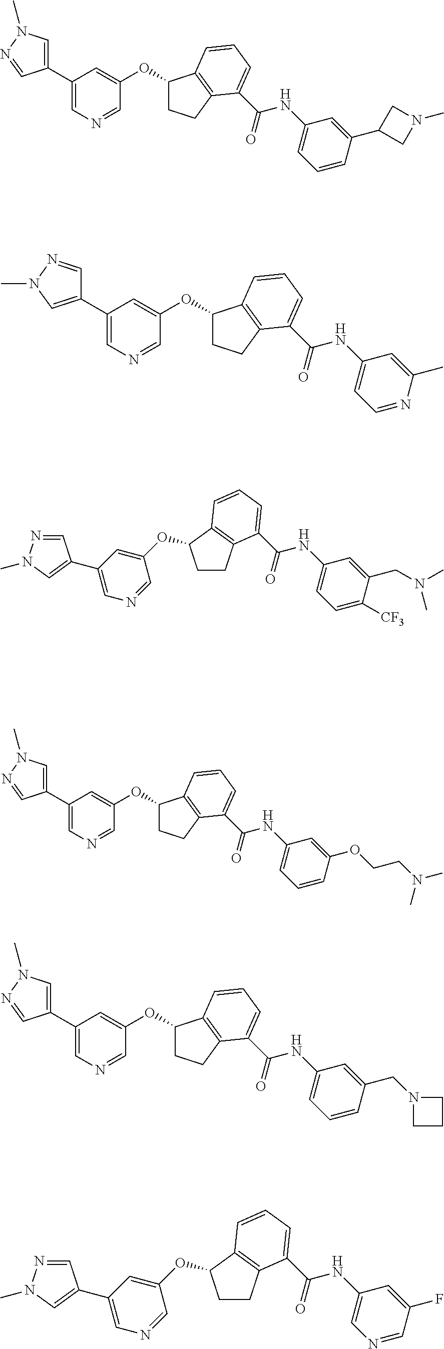

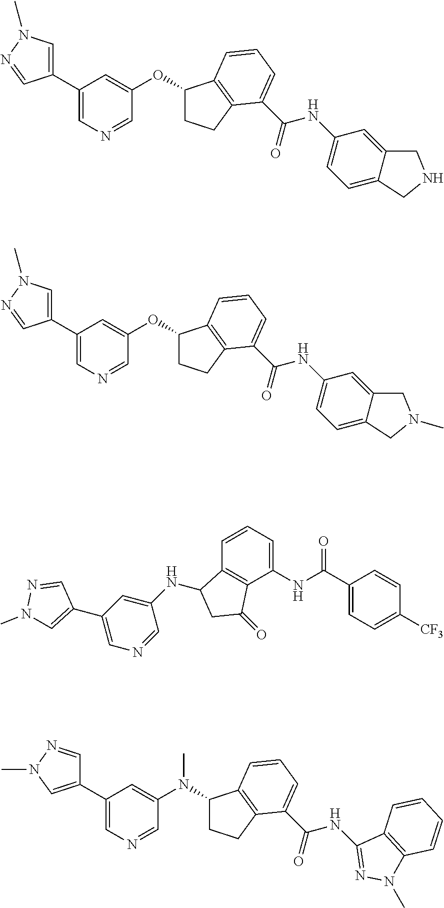

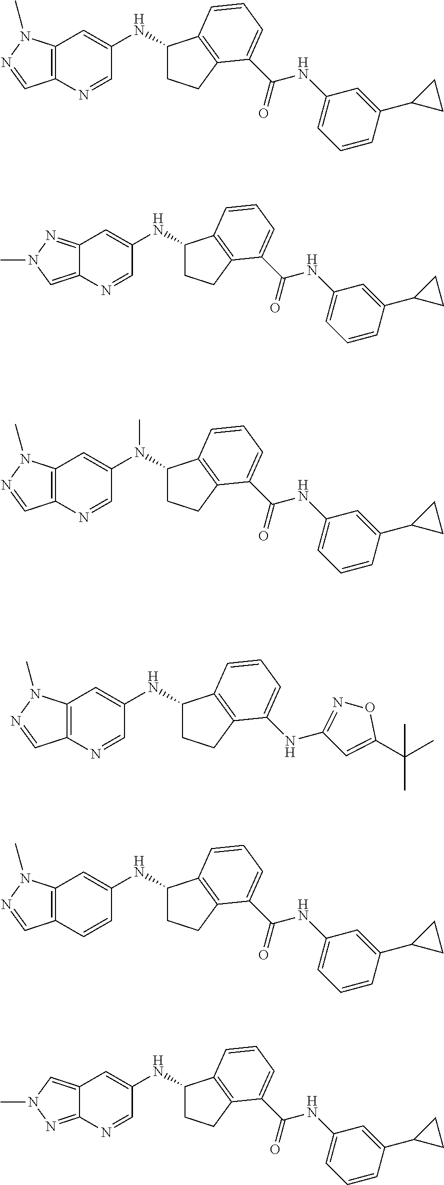

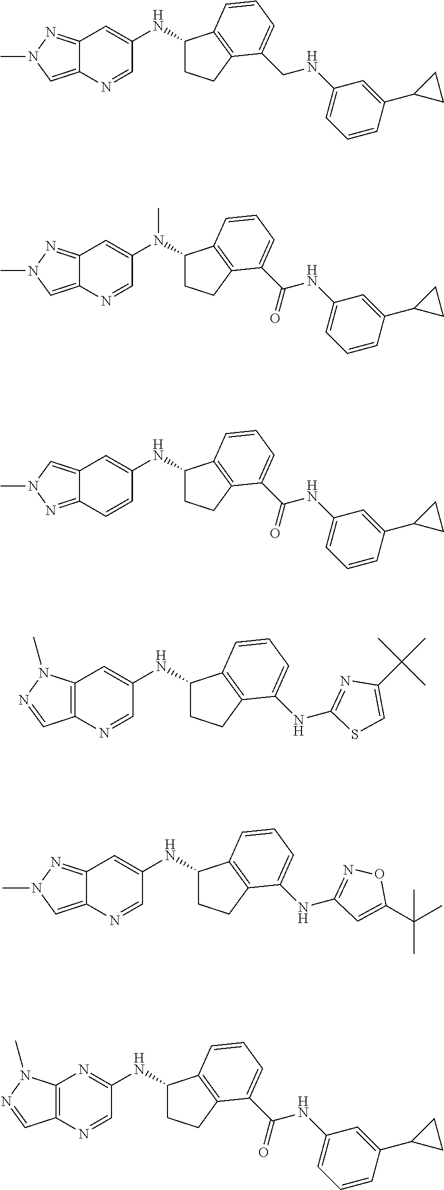

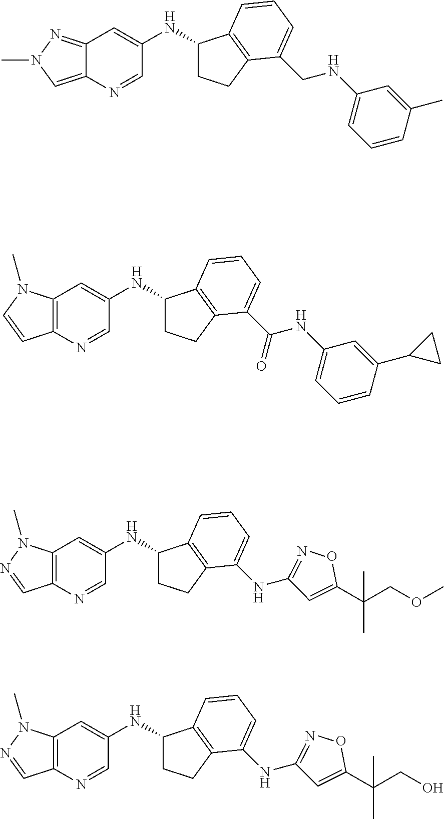

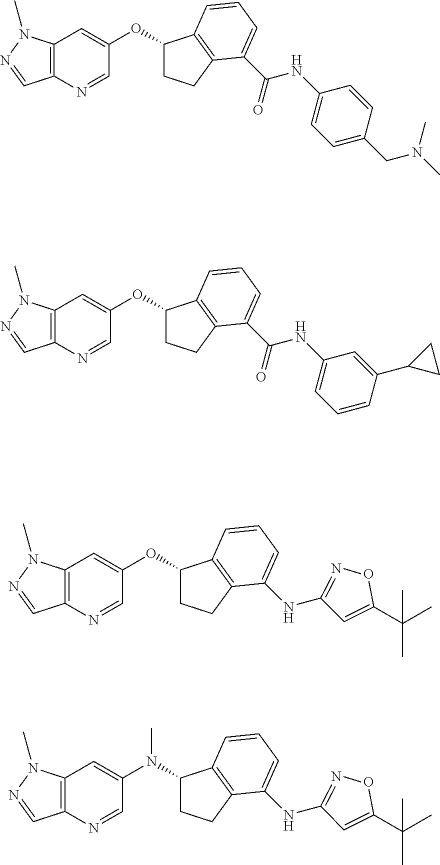 [SMILES: CN(C)Cc1ccc(NC(=O)c2cccc3c2CC[C@@H]3Oc2cnc3cnn(C)c3c2)cc1 ; Cn1ncc2ncc(O[C@H]3CCc4c(C(=O)Nc5cccc(C6CC6)c5)cccc43)cc21 ; Cn1ncc2ncc(O[C@H]3CCc4c(Nc5cc(C(C)(C)C)on5)cccc43)cc21 ; CN(c1cnc2cnn(C)c2c1)[C@H]1CCc2c(Nc3cc(C(C)(C)C)on3)cccc21]

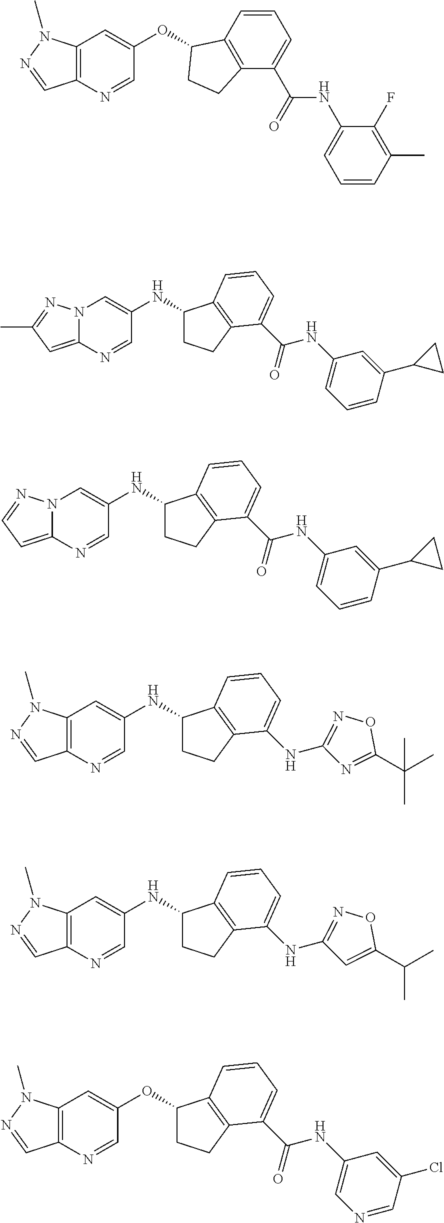

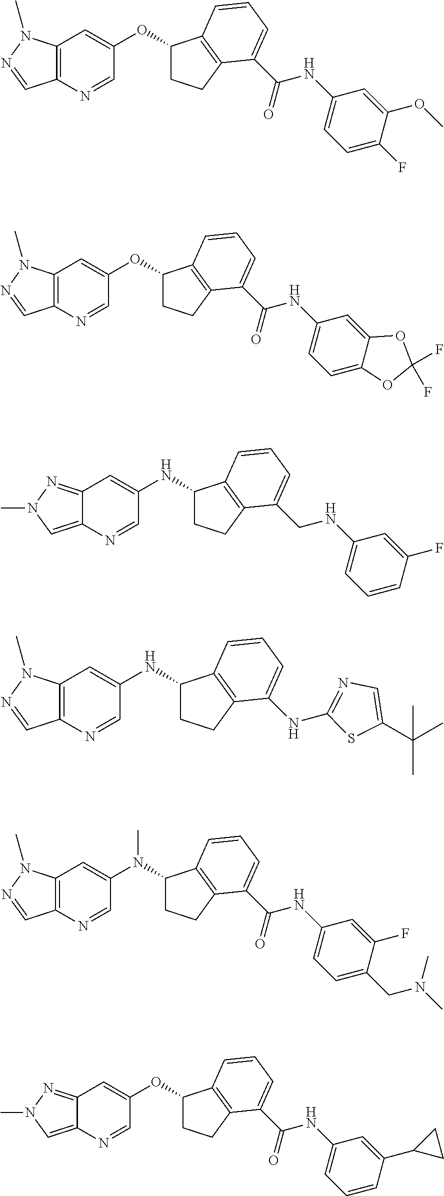

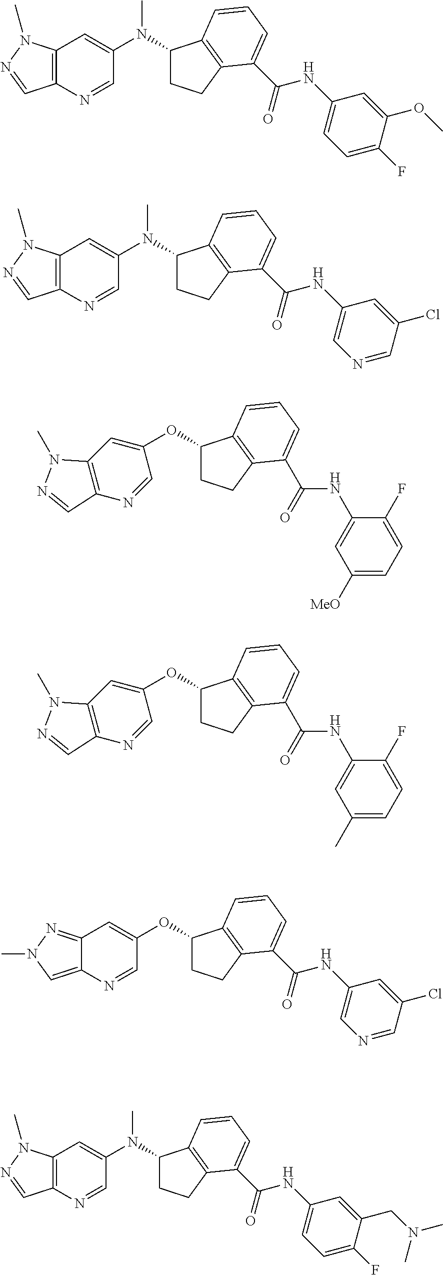

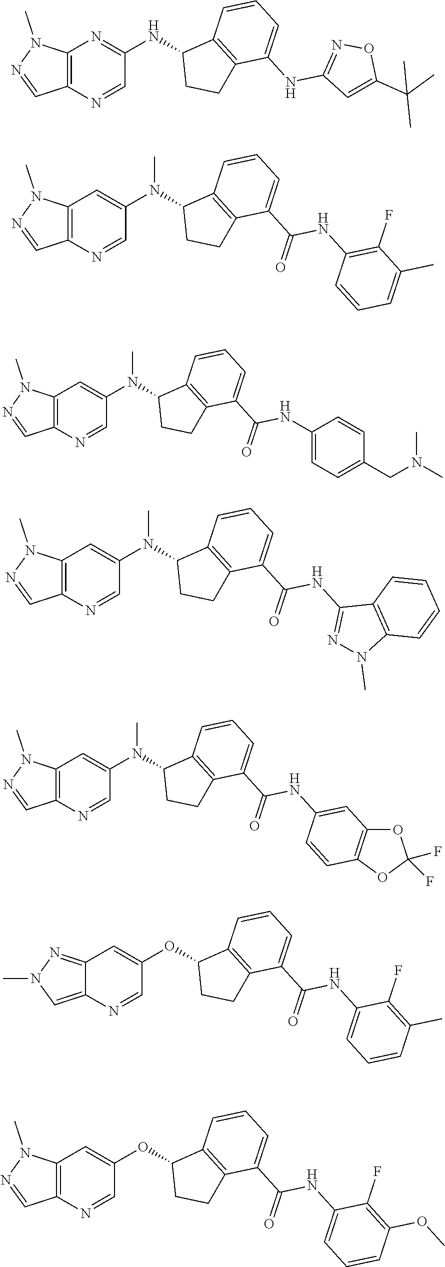

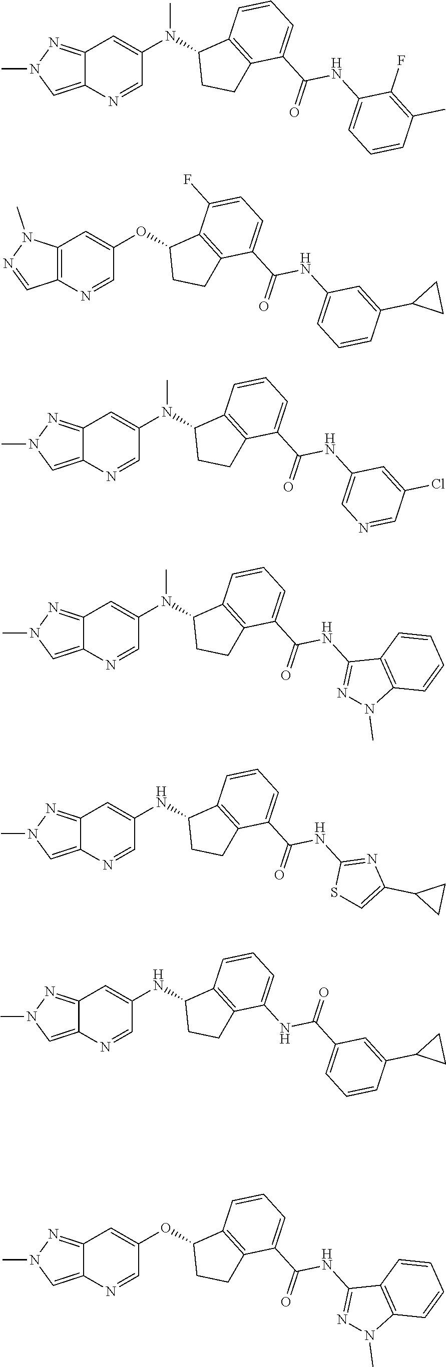

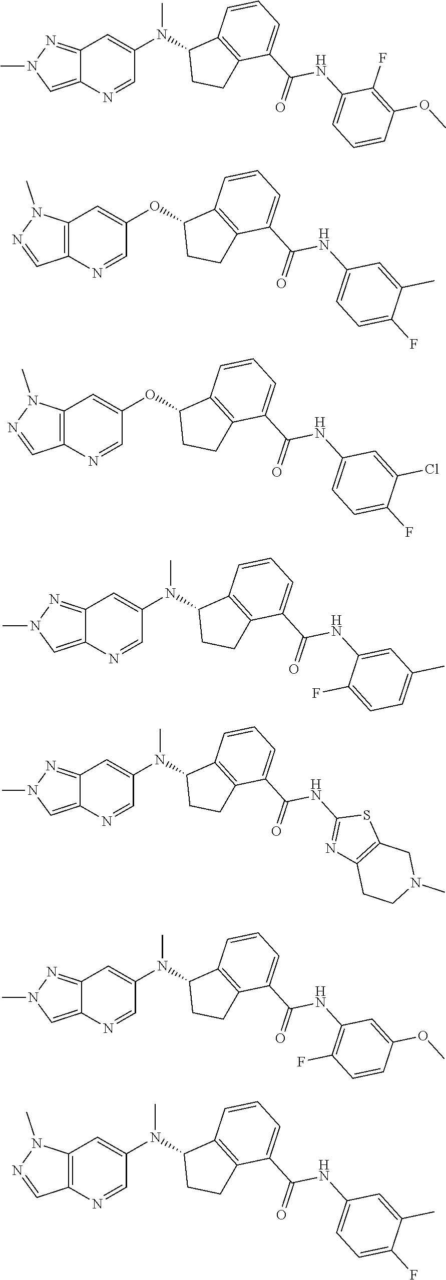

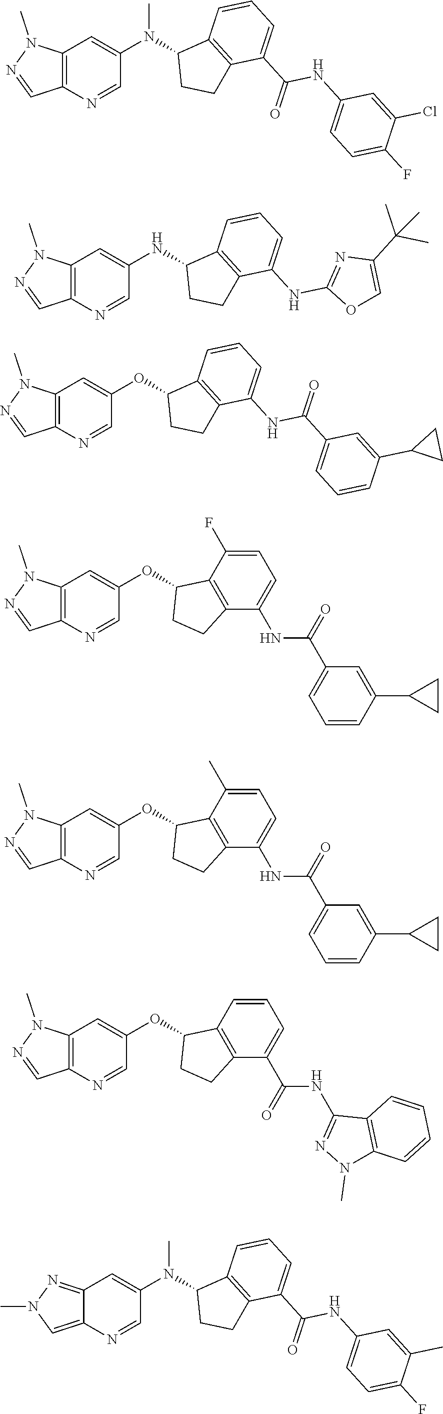

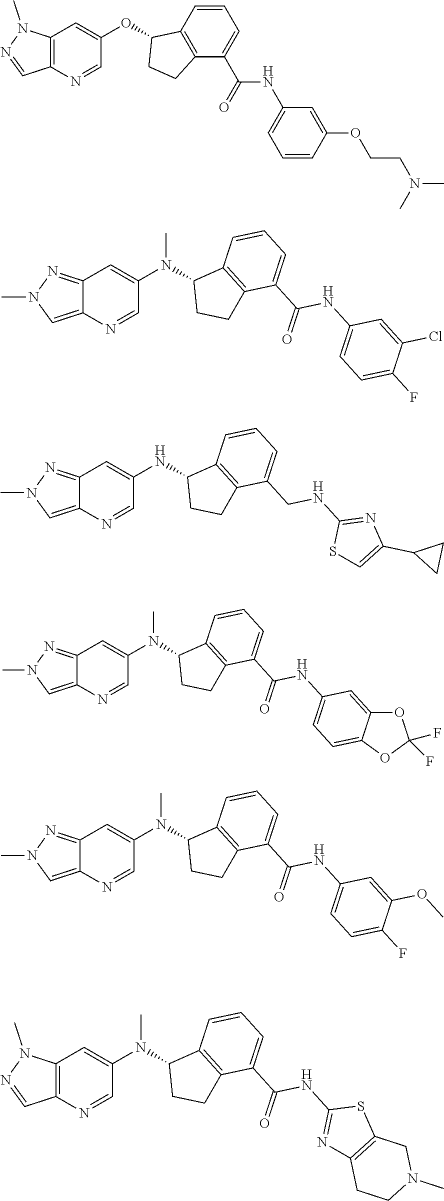

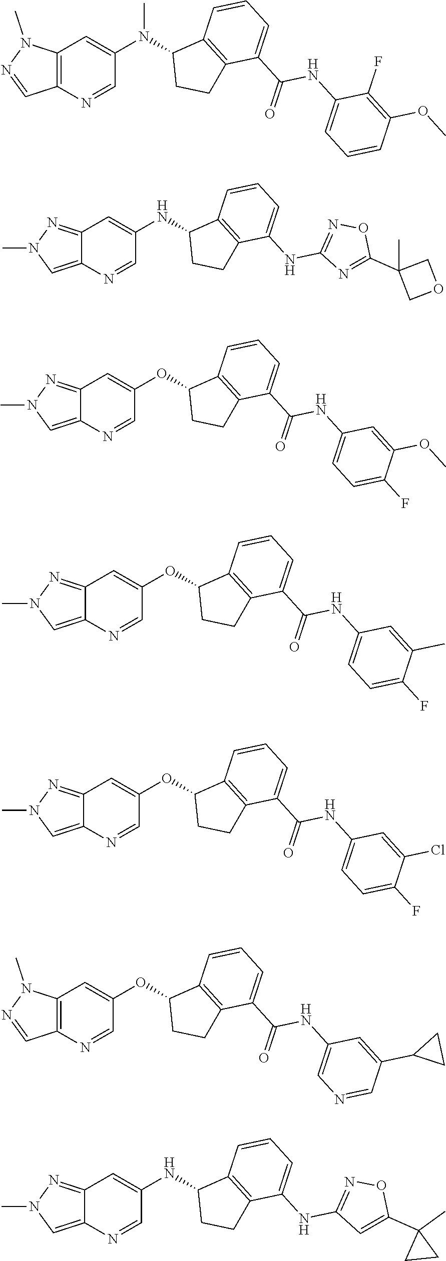

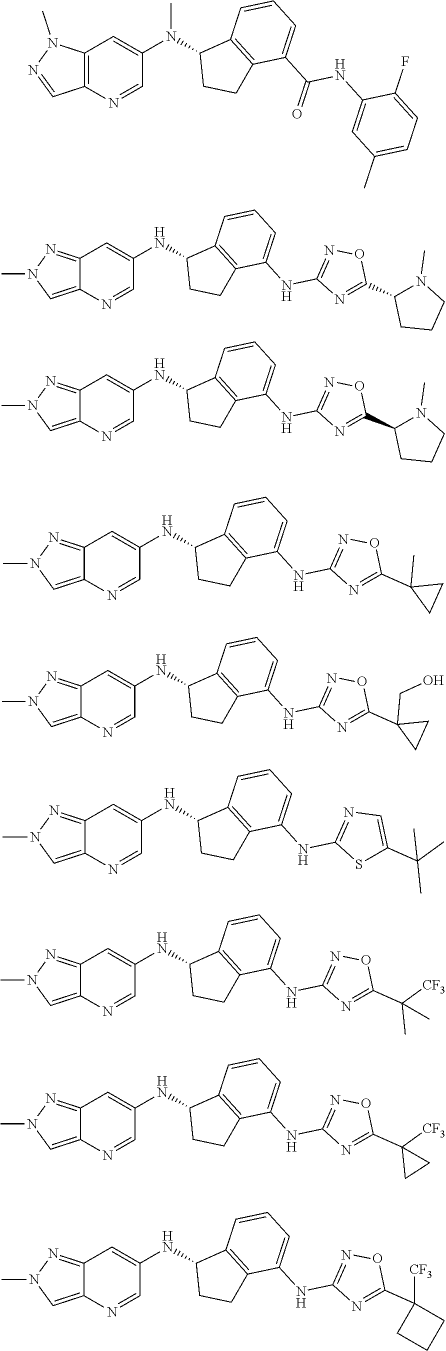

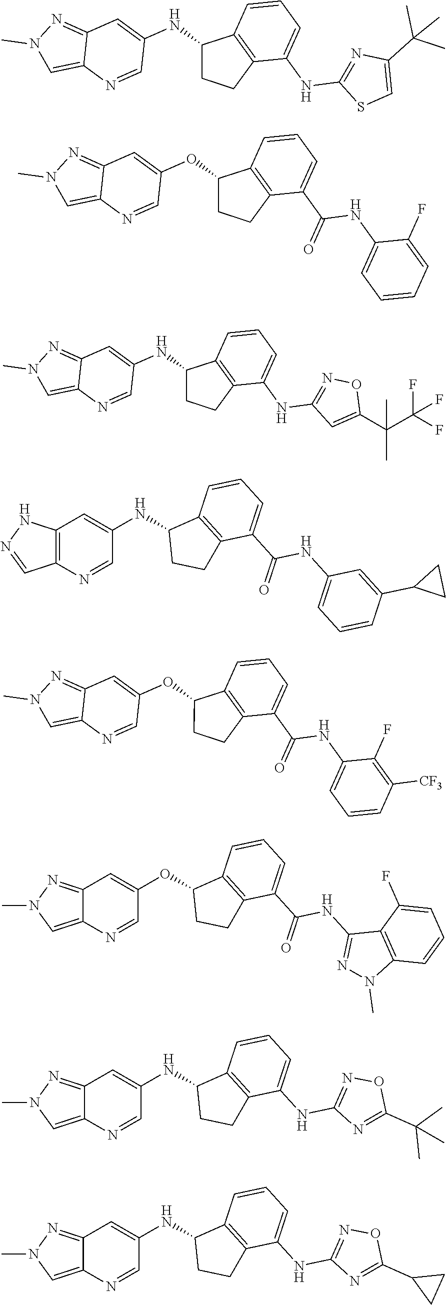

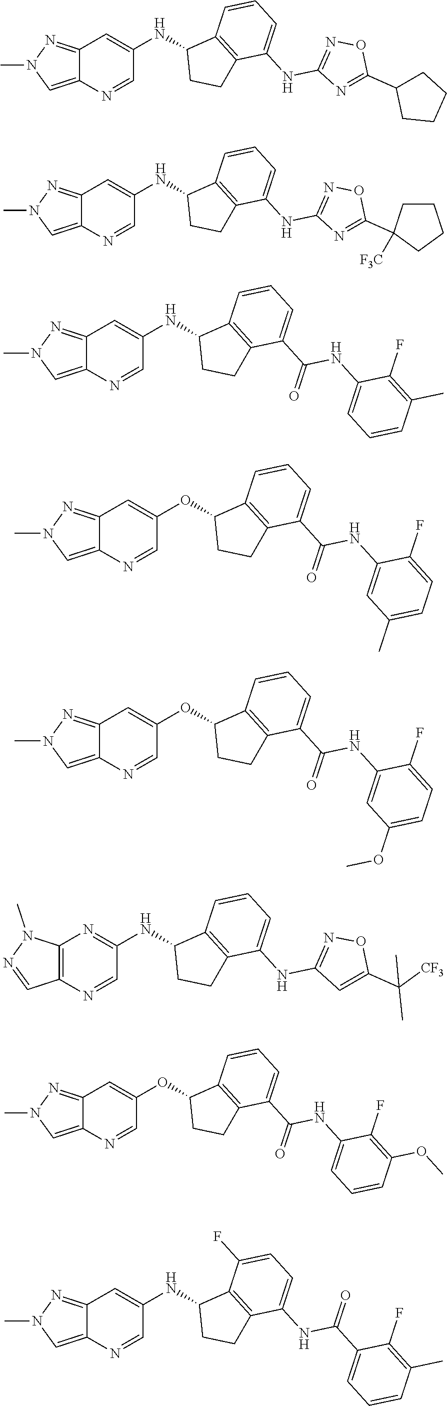

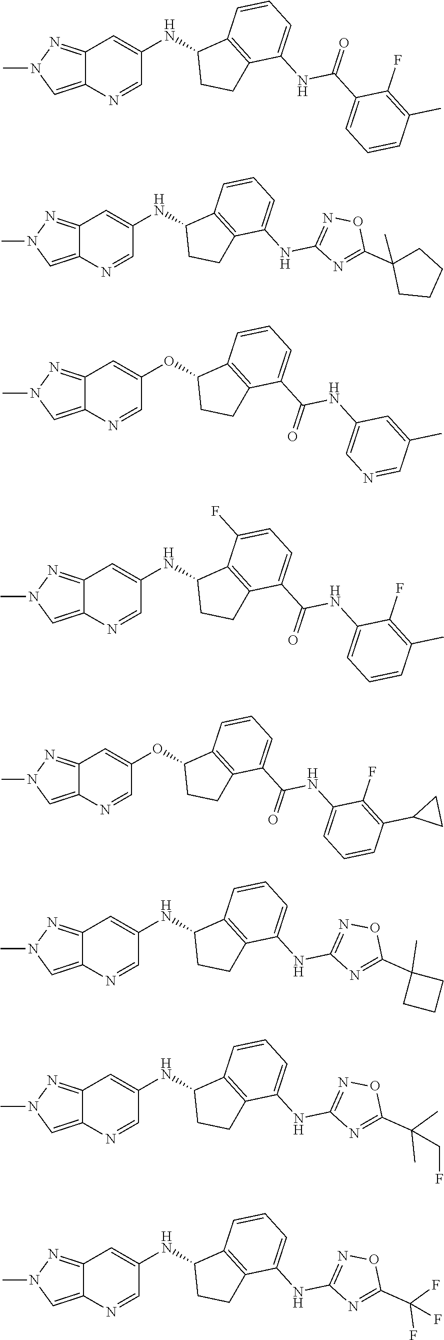

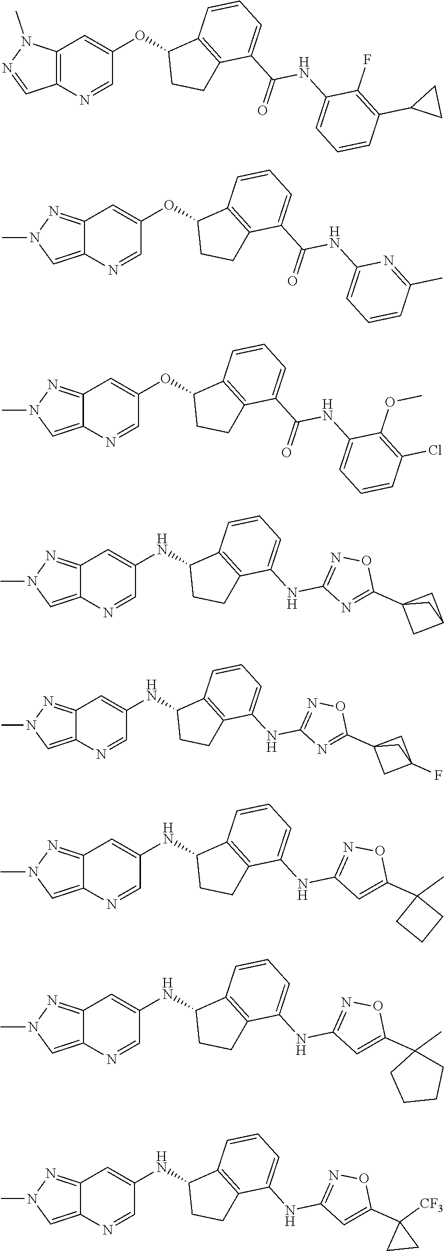

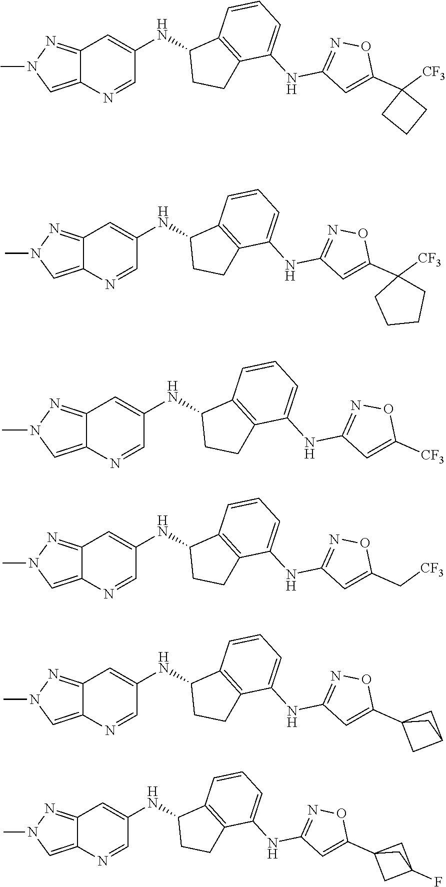 [SMILES: Cn1cc2ncc(N[C@H]3CCc4c(Nc5cc(C6(C(F)(F)F)CCC6)on5)cccc43)cc2n1 ; Cn1cc2ncc(N[C@H]3CCc4c(Nc5cc(C6(C(F)(F)F)CCCC6)on5)cccc43)cc2n1 ; Cn1cc2ncc(N[C@H]3CCc4c(Nc5cc(C(F)(F)F)on5)cccc43)cc2n1 ; Cn1cc2ncc(N[C@H]3CCc4c(Nc5cc(CC(F)(F)F)on5)cccc43)cc2n1 ; Cn1cc2ncc(N[C@H]3CCc4c(Nc5cc(C67CC(C6)C7)on5)cccc43)cc2n1 ; Cn1cc2ncc(N[C@H]3CCc4c(Nc5cc(C67CC(F)(C6)C7)on5)cccc43)cc2n1]

In another aspect, the present disclosure provides a compound having the structure of formula (III) or a pharmaceutically acceptable salt, stereoisomers, isotopic isomers thereof,

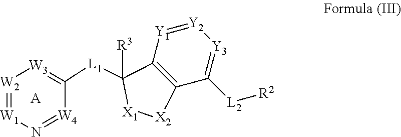

wherein, X₁ represents O, S, NR^{a}, (CR^{L}R^{L′})ₘ, C(O);
wherein, X₂ represents O, S, NR^{a}, (CR^{T}R^{T′})ₙ, C(O);
wherein, W₁ independently represents CR^{W1} or N;
wherein, W₂ independently represents CR^{W2} or N;
wherein, W₃ independently represents CR^{W3} or N;
wherein, W₄ independently represents CR^{W4} or N;
wherein, Y₁ independently represents CR^{Y1} or N;
wherein, Y₂ independently represents CR^{Y2} or N;
wherein, Y₃ independently represents CR^{Y3} or N;
wherein, each R^{W1}, R^{W2}, R^{W3}, R^{W4}, R^{Y1}, R^{Y2}, R^{Y3} independently represents hydrogen, deuterium, C₁-C₆ alkyl, deuterated C₁-C₆ alkyl, halogen, nitro, —OR^{a}, —SR^{a}, —P(O)R^{a}R^{b}, —CN, —S(O)₂R^{a}, —S(O)R^{a}, —SF₅, —NR^{a}R^{b}, —CR^{a}R^{b}NR^{a}R^{b}, —NR^{a}COR^{b}, C₁-C₆ haloalkyl, C₁-C₆ haloalkoxy, C₁-C₆ haloalkylthio, hydroxy (C₁-C₆ alkyl); or each R^{W1}, R^{W2}, R^{W3}, R^{W4}, R^{Y1}, R^{Y2}, R^{Y3} independently represents C₃-C₁₀ cycloalkyl, C₆-C₁₀cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
or W₁ and W₂ together with the atom which they are attached to form a 5-6 membered saturated or unsaturated ring fused to ring A, and the said ring may optionally contain 0, 1, 2 or 3 heteroatoms selected from O, N, or S;
wherein, L₁ represents absent, —(CR^{a}R^{b})ₒ—, —CR^{a}R^{b}O—, —OCR^{a}R^{b}—, —O—, —S—, —S(O)₂—, —C(O)—, —NR^{a}—, —S(O)₂NR^{a}—, —OC(O)—, —C(O)O—, —NR^{a}S(O)₂—, —CR^{a}R^{b}NR^{a}—, or —NR^{a}CR^{a}R^{b}—;
wherein, L₂ represents absent, —(CR^{a}R^{b})ₒ—, —CR^{a}R^{b}O—, —OCR^{a}R^{b}—, —O—, —C(O)—, —NR^{a}—, —S(O)₂NR²—, —OC(O)—, —C(O)O—, —NR^{a}S(O)₂—, —CR^{a}R^{b}NR^{a}—, —NR^{a}CR^{a}R^{b}—, —NR^{a}C(O)NR^{b}—, —NR^{a}C(O)NR^{b}CR^{a}R^{b}—, —NR^{a}C(O)O—, —NR^{a}C(O)—, or —C(O)NR^{a}—;
wherein, R² represents C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl, —CR^{a}R^{b}C₆-C₁₀ aryl, —CR^{a}R^{b}(5-10 membered heteroaryl); which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, C₆-C₁₀ aryl, 5-10 membered heteroaryl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —S(O)₂R^{a}, —S(O)R^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, R³ represents hydrogen, halogen, C₁-C₆ haloalkyl, C₁-C₆ alkyl, or hydroxy;
or R³ and W₃ together with the atom which they are attached to form a 5-7 membered saturated or unsaturated ring fused to ring A, and the said ring may optionally contain 0, 1 or 2 heteroatoms selected from O, N, or S; and further, the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, each R^{a} and R^{b} independently represents hydrogen, deuterium, halogen, C₁-C₆ alkyl, C₁-C₆ haloalkyl, hydroxy (C₁-C₆ alkyl), C₃-C₁₀ cycloalkyl; or R^{a} and R^{b} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, each R^{L} and R^{L′} independently represents hydrogen, deuterium, halogen, C₁-C₆ alkyl, C₁-C₆ haloalkyl, hydroxy (C₁-C₆ alkyl), C₃-C₁₀ cycloalkyl; or R^{L} and R^{L′} together with the carbon atom which they are attached to form a 3-6 membered ring, and the said ring may optionally contain 0, 1 or 2 heteroatoms selected from O, N, or S; and further, the said ring may be optionally substituted by halogen, hydroxy or C₁-C₆ alkyl;
wherein, each R^{T} and R^{T′} independently represents hydrogen, deuterium, halogen, C₁-C₆ alkyl, C₁-C₆ haloalkyl, hydroxy (C₁-C₆ alkyl), C₃-C₁₀ cycloalkyl; or R^{T} and R^{T′} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, o, m, n represents the integer of 1, 2 or 3.

In some embodiments of the present disclosure, wherein W₁ represents CH, CCH₃, CF, CCF₃, CCHF₂, CCH₂F, C(OCH₃), CNH₂, CNHCH₃, CN(CH₃)₂, CCH₂NHCH₃, CCH₂N(CH₃)₂, C(CN) or N.

In some embodiments of the present disclosure, wherein W₃ represents CH, CCH₃, CF, CCF₃, CCHF₂, CCH₂F, C(OCH₃), CNH₂, CNHCH₃, C(CN) or N.

In some embodiments of the present disclosure, wherein W₄ represents CH, CCH₃, CF, CCF₃, CCHF₂, CCH₂F, C(OCH₃), CNH₂, CNHCH₃, C(CN) or N.

In some embodiments of the present disclosure, wherein Y₁ represents CH, CCH₃, CF, —CCl, CCF₃, CCHF₂, CCH₂F, C(OCH₃), C(CN) or N.

In some embodiments of the present disclosure, wherein Y₂ represents CH, CCH₃, CF, —CCl, CCF₃, CCHF₂, CCH₂F, C(OCH₃), C(CN) or N.

In some embodiments of the present disclosure, wherein Y₃ represents CH, CCH₃, CF, —CCl, CCF₃, CCHF₂, CCH₂F, C(OCH₃), C(CN) or N.

In some embodiments of the present disclosure, wherein X₁ represents —CH₂—, —CF₂—, —CHF—, —O—, —S—, —CHOH—, —CH₂CH₂— or —C(CH₃)₂—.

In some embodiments of the present disclosure, wherein X₂ represents —CH₂—, —CF₂—, —CHF—, —O—, —S—, —CHOH—, —CH₂CH₂— or —C(CH₃)₂—.

In some embodiments of the present disclosure, wherein L₁ represents —CH₂—, —CHF—, —CF₂—, —O—, —NH— or —NCH₃—.

In some embodiments of the present disclosure, wherein L₂ represents —NHC(O)—, —C(O)NH—, —NHC(O)NH—, —NHC(O)O—, —CH₂NH—, —NHCH₂—, —CH₂O—, —OCH₂— or —NH—.

In some embodiments of the present disclosure, wherein L₂ represents —NHC(O)—, —NHC(O)NH—, —NHC(O)O—, —CH₂NH—, —NHCH₂—, —CH₂O—, —OCH₂— or —NH—.

In some embodiments of the present disclosure, wherein L₂ represents —C(O)NH—.

In some embodiments of the present disclosure, wherein R^{W2} represents C₁-C₆ alkyl, halogen, —OH, O(C₁-C₆ alkyl); or R^{W2} represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —N^{a}COR^{b} or —CONR^{a}R^{b}.

In some embodiments of the present disclosure, wherein R^{W2} represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein R^{W2} represents C₃-C₁₀ cycloalkyl, C₆-C₁₀cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, —OR^{a}, hydroxy (C₁-C₆ alkyl), —CN, C₁-C₆ haloalkyl.

In some embodiments of the present disclosure, wherein R^{W2} represents 5-10 membered heteroaryls, which are substituted by 0-4 substituents selected from deuterium, halogen, C₁-C₆ alkyl, —OR^{a}, hydroxy (C₁-C₆ alkyl), —CN, C₁-C₆ haloalkyl.

In some embodiments of the present disclosure, wherein R^{W2} represents pyrazolyl, triazolyl, oxazolyl, thiazolyl, imidazolyl, which are substituted by 0-4 substituents selected from deuterium, halogen, C₁-C₆ alkyl, —OR^{a}, hydroxy (C₁-C₆ alkyl), —CN, C₁-C₆ haloalkyl.

In some embodiments of the present disclosure, wherein R^{W2} represents

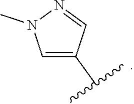 [SMILES: *c1cnn(C)c1]

In some embodiments of the present disclosure, wherein R² represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, phenyl, 5-6 membered heteroaryl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein L₂ represents —NHC(O)—, —NHC(O)NH—, —NHC(O)O—, —CH₂NH—, —NHCH₂—, —CH₂O—, —OCH₂— or —NH—; R² represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, phenyl, 5-6 membered heteroaryl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein R² represents C₃-C₁₀ cycloalkyl, C₆-C₁₀ cycloalkenyl, 4-10 membered heterocycloalkyl, 6-10 membered heterocycloalkenyl, C₆-C₁₀ aryl, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein L₂ represents —C(O)NH—; R² represents C₃-C₁₀ cycloalkyl, C₆-C₁₀ cycloalkenyl, 4-10 membered heterocycloalkyl, 6-10 membered heterocycloalkenyl, C₆-C₁₀ aryl, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein W₁ and W₂ combine to form a ring fused to ring A having the following structure:

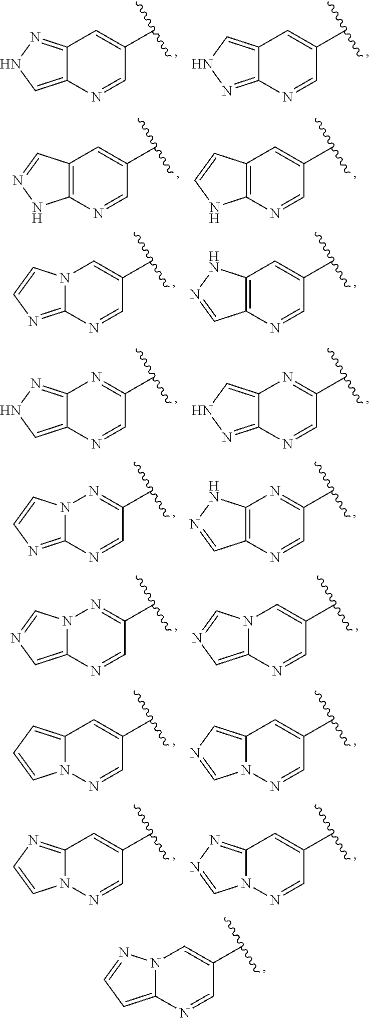

wherein the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}; and wherein the wavy line indicates the point of attachment with L₁.

In some embodiments of the present disclosure, wherein W₁ and W₂ combine to form a ring fused to ring A having the following structure:

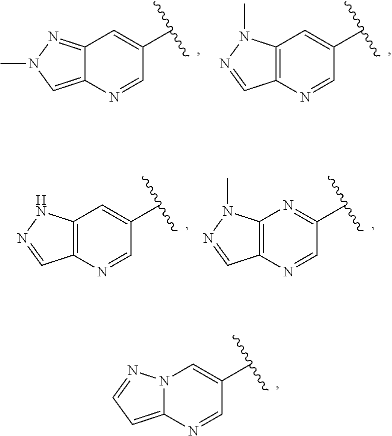 [SMILES: *c1cnc2cn(C)nc2c1 ; *c1cnc2cnn(C)c2c1 ; *c1cnc2cn[nH]c2c1 ; *c1cnc2cnn(C)c2n1 ; *c1cnc2ccnn2c1]

wherein the wavy line indicates the point of attachment of L₁.

In some embodiments of the present disclosure, wherein R² represents phenyl, pyridinyl, indazolyl, indolyl, thiazolyl, isothiazolyl, imidazolyl, pyrazolyl, oxazolyl, isoxazolyl, 1,2,4-oxadiazolyl, 1,3,4-oxadiazolyl, pyridazinyl, piperazinyl, pyrazinyl, cyclohexyl, benzothienyl, 1,2-benzoisoxazolyl, benzo[d] isothiazolyl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, phenyl, 5-6 membered heteroaryl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein L₂ represents —NHC(O)—, —NHC(O)NH—, —NHC(O)O—, —CH₂NH—, —NHCH₂—, —CH₂O—, —OCH₂— or —NH—; R² represents phenyl, pyridinyl, indazolyl, indolyl, thiazolyl, isothiazolyl, imidazolyl, pyrazolyl, oxazolyl, isoxazolyl, 1,2,4-oxadiazolyl, 1,3,4-oxadiazolyl, pyridazinyl, piperazinyl, pyrazinyl, cyclohexyl, benzothienyl, 1,2-benzoisoxazolyl, benzo[d] isothiazolyl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, phenyl, 5-6 membered heteroaryl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R² represents

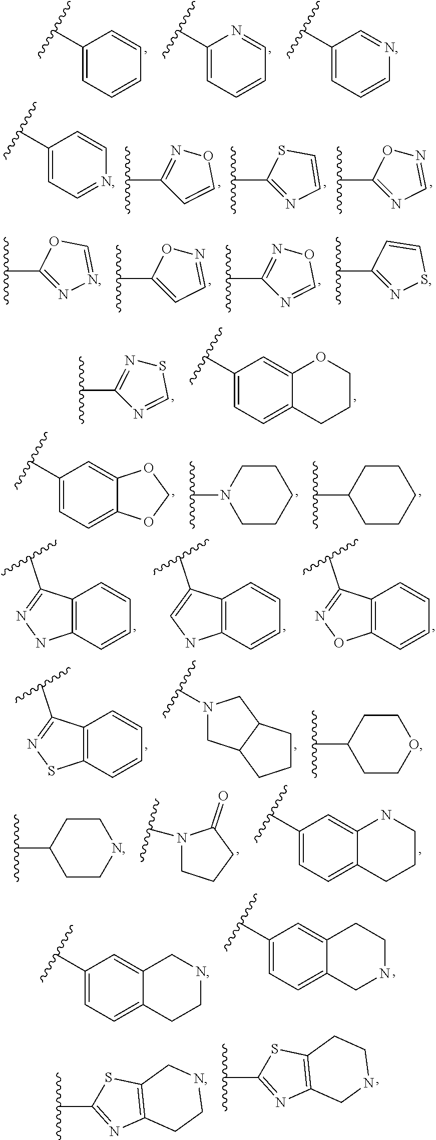

which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein L₂ represents —NHC(O)—, —NHC(O)NH—, —NHC(O)O—, —CH₂NH—, —NHCH₂—, —CH₂O—, —OCH₂— or —NH—; R² represents

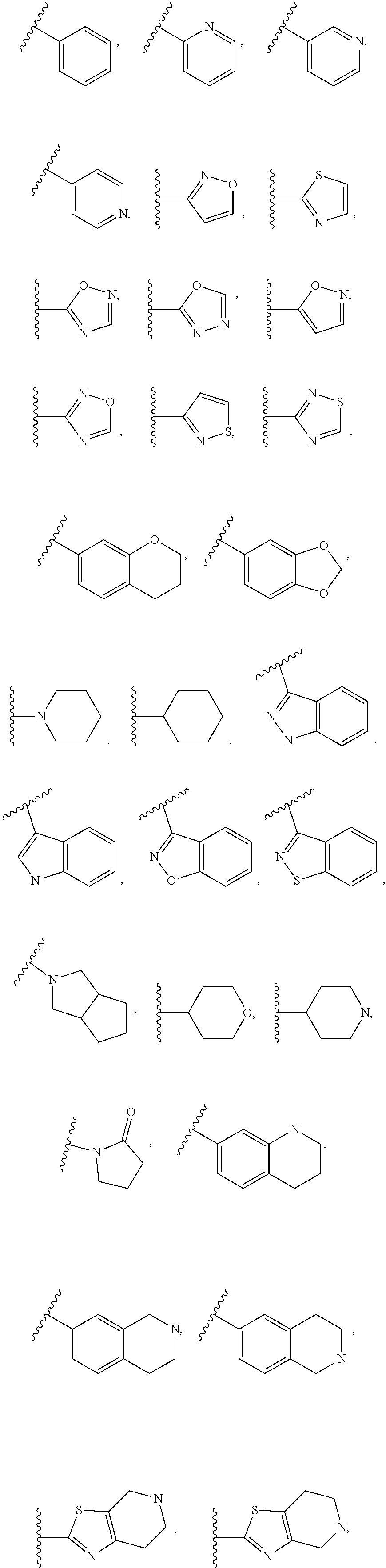

which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R² represents:

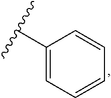 [SMILES: *c1ccccc1]

which is substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆haloalkyl, or C₁-C₆ alkoxy.

In some embodiments of the present disclosure, wherein L₂ represents —C(O)NH—; R² represents:

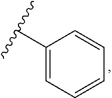 [SMILES: *c1ccccc1]

which is substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R³ represents hydrogen or CH₃.

The present disclosure provides a compound having the structure of formula (III) or a pharmaceutically acceptable salt, stereoisomers, isotopic isomers thereof,

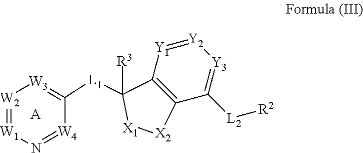

wherein, X₁ represents O, S, NR^{a}, (CR^{L}R^{L′})ₘ, C(O);
wherein, X₂ represents O, S, NR^{a}, (CR^{T}R^{T′})ₙ, C(O);
wherein, W₁ independently represents CR^{W1} or N;
wherein, W₂ independently represents CR^{W2};
wherein, W₃ independently represents CR^{W3} or N;
wherein, W₄ independently represents CR^{W4} or N;
wherein, Y₁ independently represents CR^{Y1} or N;
wherein, Y₂ independently represents CR^{Y2} or N;
wherein, Y₃ independently represents CR^{Y3} or N;
wherein, each R^{W1}, R^{W2}, R^{W3}, R^{W4}, R^{Y1}, R^{Y2}, R^{Y3} independently represents hydrogen, deuterium, C₁-C₆ alkyl, deuterated C₁-C₆ alkyl, halogen, nitro, —OR^{a}, —SR^{a}, —P(O)R^{a}R^{b}, —CN, —S(O)₂R^{a}, —S(O)R^{a}, —SF₅, —NR^{a}R^{b}, —CR^{a}R^{b}NR^{a}R^{b}, —NR^{a}COR^{b}, C₁-C₆ haloalkyl, C₁-C₆ haloalkoxy, C₁-C₆ haloalkylthio, hydroxy (C₁-C₆ alkyl); or each R^{W1}, R^{W2}, R^{W3}, R^{W4}, R^{Y1}, R^{Y2}, R^{Y3} independently represents C₃-C₁₀ cycloalkyl, C₆-C₁₀cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR³R^{b}, —NR^{a}COR^{b} or —CONR^{a}R^{b}.
or W₁ and W₂ together with the atom which they are attached to form a 5-6 membered saturated or unsaturated ring fused to ring A, and the said ring may optionally contain 0, 1, 2 or 3 heteroatoms selected from O, N, or S;
wherein, L₁ represents absent, —(CR^{a}R^{b})ₒ—, —CR^{a}R^{b}O—, —OCR^{a}R^{b}—, —O—, —S—, —S(O)₂—, —C(O)—, —NR^{a}—, —S(O)₂NR^{a}—, —OC(O)—, —C(O)O—, —NR^{a}S(O)₂—, —CR^{a}R^{b}NR^{a}—, or —NR^{a}CR^{a}R^{b}—;
wherein, L₂ represents absent, —(CR^{a}R^{b})ₒ—, —CR^{a}R^{b}O—, —OCR^{a}R^{b}—, —O—, —C(O)—, —NR^{a}—, —S(O)₂NR^{a}—, —OC(O)—, —C(O)O—, —NR^{a}S(O)₂—, —CR^{a}R^{b}NR^{a}—, —NR^{a}CR^{a}R^{b}—, —NR^{a}C(O)NR^{b}—, —NR^{a}C(O)NR^{b}CR^{a}R^{b}—, —NR^{a}C(O)O—, —NR^{a}C(O)—, or —C(O)NR^{a}—;
wherein, R² represents C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl, —CR^{a}R^{b}C₆-C₁₀ aryl, —CR^{a}R^{b}(5-10 membered heteroaryl); which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, C₆-C₁₀ aryl, 5-10 membered heteroaryl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —S(O)₂R^{a}, —S(O)R^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, R³ represents hydrogen, halogen, C₁-C₆ haloalkyl, C₁-C₆ alkyl, or hydroxy;
or R³ and W₃ together with the atom which they are attached to form a 5-7 membered saturated or unsaturated ring fused to ring A, and the said ring may optionally contain 0, 1 or 2 heteroatoms selected from O, N, or S; and further, the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, each R^{a} and R^{b} independently represents hydrogen, deuterium, halogen, C₁-C₆ alkyl, C₁-C₆ haloalkyl, hydroxy (C₁-C₆ alkyl), C₃-C₁₀ cycloalkyl; or R^{a} and R^{b} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, each R^{L} and R^{L′} independently represents hydrogen, deuterium, halogen, C₁-C₆ alkyl, C₁-C₆ haloalkyl, hydroxy (C₁-C₆ alkyl), C₃-C₁₀ cycloalkyl; or R^{L} and R^{L′} together with the carbon atom which they are attached to form a 3-6 membered ring, and the said ring may optionally contain 0, 1 or 2 heteroatoms selected from O, N, or S; and further, the said ring may be optionally substituted by halogen, hydroxy or C₁-C₆ alkyl;
wherein, each R^{T} and R^{T′} independently represents hydrogen, deuterium, halogen, C₁-C₆ alkyl, C₁-C₆ haloalkyl, hydroxy (C₁-C₆ alkyl), C₃-C₁₀ cycloalkyl; or R^{T} and R^{T′} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, o, m, n represents the integer of 1, 2 or 3.

In some embodiments of the present disclosure, wherein W₁ represents CH, CCH₃, CF, CCF₃, CCHF₂, CCH₂F, C(OCH₃), CNH₂, CNHCH₃, CN(CH₃)₂, CCH₂NHCH₃, CCH₂N(CH₃)₂, C(CN) or N.

In some embodiments of the present disclosure, wherein W₃ represents CH, CCH₃, CF, CCF₃, CCHF₂, CCH₂F, C(OCH₃), CNH₂, CNHCH₃, C(CN) or N.

In some embodiments of the present disclosure, wherein W₄ represents CH, CCH₃, CF, CCF₃, CCHF₂, CCH₂F, C(OCH₃), CNH₂, CNHCH₃, C(CN) or N.

In some embodiments of the present disclosure, wherein Y₁ represents CH, CCH₃, CF, —CCl, CCF₃, CCHF₂, CCH₂F, C(OCH₃), C(CN) or N.

In some embodiments of the present disclosure, wherein Y₂ represents CH, CCH₃, CF, —CCl, CCF₃, CCHF₂, CCH₂F, C(OCH₃), C(CN) or N.

In some embodiments of the present disclosure, wherein Y₃ represents CH, CCH₃, CF, —CCl, CCF₃, CCHF₂, CCH₂F, C(OCH₃), C(CN) or N.

In some embodiments of the present disclosure, wherein X₁ represents —CH₂—, —CF₂—, —CHF—, —O—, —S—, —CHOH—, —CH₂CH₂— or —C(CH₃)₂—.

In some embodiments of the present disclosure, wherein X₂ represents —CH₂—, —CF₂—, —CHF—, —O—, —S—, —CHOH—, —CH₂CH₂— or —C(CH₃)₂—.

In some embodiments of the present disclosure, wherein L₁ represents —CH₂—, —CHF—, —CF₂—, —O—, —NH— or —NCH₃—.

In some embodiments of the present disclosure, wherein L₂ represents —NHC(O)—, —C(O)NH—, —NHC(O)NH—, —NHC(O)O—, —CH₂NH—, —NHCH₂—, —CH₂O—, —OCH₂— or —NH—.

In some embodiments of the present disclosure, wherein L₂ represents —NHC(O)—, —NHC(O)NH—, —NHC(O)O—, —CH₂NH—, —NHCH₂—, —CH₂O—, —OCH₂— or —NH—.

In some embodiments of the present disclosure, wherein L₂ represents —C(O)NH—.

In some embodiments of the present disclosure, wherein R¹² represents C₁-C₆ alkyl, halogen, —OH, O(C₁-C₆ alkyl); or R^{W2} represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substitutes selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), NR^{a}R^{b}, —CR^{a}R^{b}NR³R, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein R^{W2} represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substitutes selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein R¹ represents C₃-C₁₀ cycloalkyl, C₆-C₁₀cycloalkenyl, 4-10 membered heterocycloalkyl, 6-10 membered heterocycloalkenyl, C₆-C₁₀ aryl, 5-10 membered heteroaryl, which are substituted by 0-4 substitutes selected from deuterium, halogen, C₁-C₆ alkyl, —OR^{a}, hydroxy (C₁-C₆ alkyl), —CN, C₁-C₆ haloalkyl.

In some embodiments of the present disclosure, wherein R^{W2} represents 5-10 membered heteroaryls, which are substituted by 0-4 substitutes selected from deuterium, halogen, C₁-C₆ alkyl, —OR^{a}, hydroxy (C₁-C₆ alkyl), —CN, C₁-C₆ haloalkyl.

In some embodiments of the present disclosure, wherein R^{W2} represents pyrazolyl, triazolyl, oxazolyl, thiazolyl, imidazolyl, which are substituted by 0-4 substitutes selected from deuterium, halogen, C₁-C₆ alkyl, —OR^{a}, hydroxy (C₁-C₆ alkyl), —CN, C₁-C₆ haloalkyl.

In some embodiments of the present disclosure, wherein R^{W2} represents

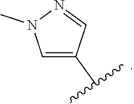 [SMILES: *c1cnn(C)c1]

In some embodiments of the present disclosure, wherein R² represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, phenyl, 5-6 membered heteroaryl, —OR³, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein L₂ represents —NHC(O)—, —NHC(O)NH—, —NHC(O)O—, —CH₂NH—, —NHCH₂—, —CH₂O—, —OCH₂— or —NH—; R² represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, phenyl, 5-6 membered heteroaryl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR⁸, —SF₅, —C(O)R^{a}, —C(O)ORV, —OC(O)R^{a}, —OC(O)NR^{a}R^{b}, —NR^{a}COR^{b} or —CONR^{a}R^{b}.

In some embodiments of the present disclosure, wherein R² represents C₃-C₁₀ cycloalkyl, C₆-C₁₀ cycloalkenyl, 4-10 membered heterocycloalkyl, 6-10 membered heterocycloalkenyl, C₆-C₁₀ aryl, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)^{a}R^{b}, —NR^{a}COR^{b} or —CONR^{a}R^{b}.

In some embodiments of the present disclosure, wherein L₂ represents —C(O)NH—; R² represents C₃-C₁₀ cycloalkyl, C₆-C₁₀ cycloalkenyl, 4-10 membered heterocycloalkyl, 6-10 membered heterocycloalkenyl, C₆-C₁₀ aryl, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein W₁ and W₂ combine to form a ring fused to ring A having the following structure:

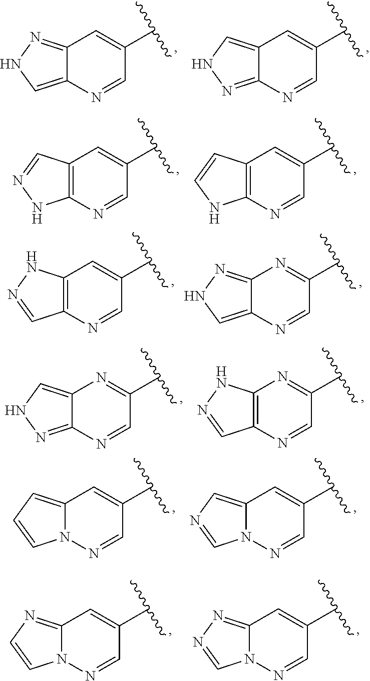

wherein the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}; and wherein the wavy line indicates the point of attachment with L₁.

In some embodiments of the present disclosure, wherein W₁ and W₂ combine to form a ring fused to ring A having the following structure:

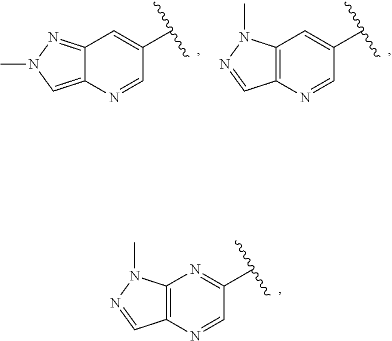 [SMILES: *c1cnc2cn(C)nc2c1 ; *c1cnc2cnn(C)c2c1 ; *c1cnc2cnn(C)c2n1]

wherein the wavy line indicates the point of attachment with L₁.

In some embodiments of the present disclosure, wherein R² represents phenyl, pyridinyl, indazolyl, indolyl, thiazolyl, isothiazolyl, imidazolyl, pyrazolyl, oxazolyl, isoxazolyl, 1,2,4-oxadiazolyl, 1,3,4-oxadiazolyl, pyridazinyl, piperazinyl, pyrazinyl, cyclohexyl, benzothienyl, 1,2-benzoisoxazolyl, benzo[d] isothiazolyl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, phenyl, 5-6 membered heteroaryl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein L₂ represents —NHC(O)—, —NHC(O)NH—, —NHC(O)O—, —CH₂NH—, —NHCH₂—, —CH₂O—, —OCH₂— or —NH—; R² represents phenyl, pyridinyl, indazolyl, indolyl, thiazolyl, isothiazolyl, imidazolyl, pyrazolyl, oxazolyl, isoxazolyl, 1,2,4-oxadiazolyl, 1,3,4-oxadiazolyl, pyridazinyl, piperazinyl, pyrazinyl, cyclohexyl, benzothienyl, 1,2-benzoisoxazolyl, benzo[d] isothiazolyl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, phenyl, o 5-6 membered heteroaryl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R² represents

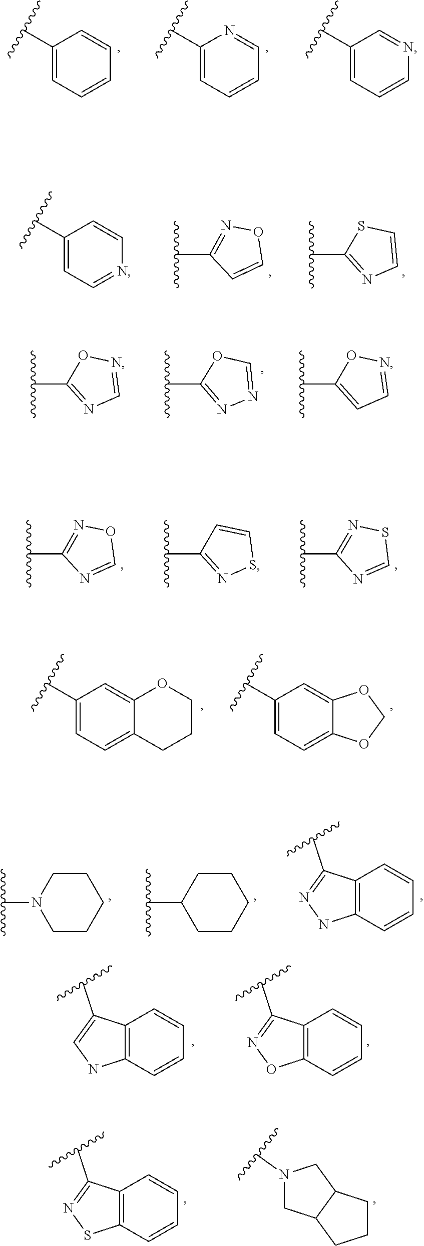

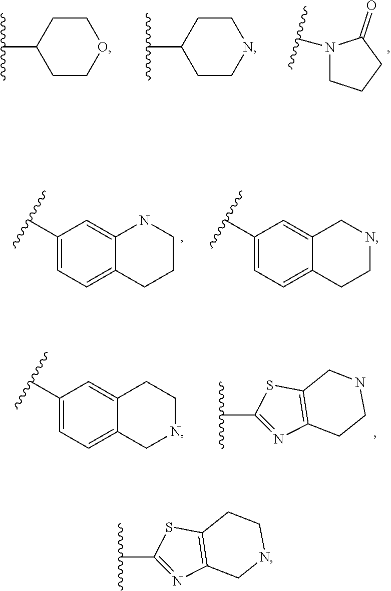

which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein L₂ represents —NHC(O)—, —NHC(O)NH—, —NHC(O)O—, —CH₂NH—, —NHCH₂—, —CH₂O—, —OCH₂— or —NH—; R² represents

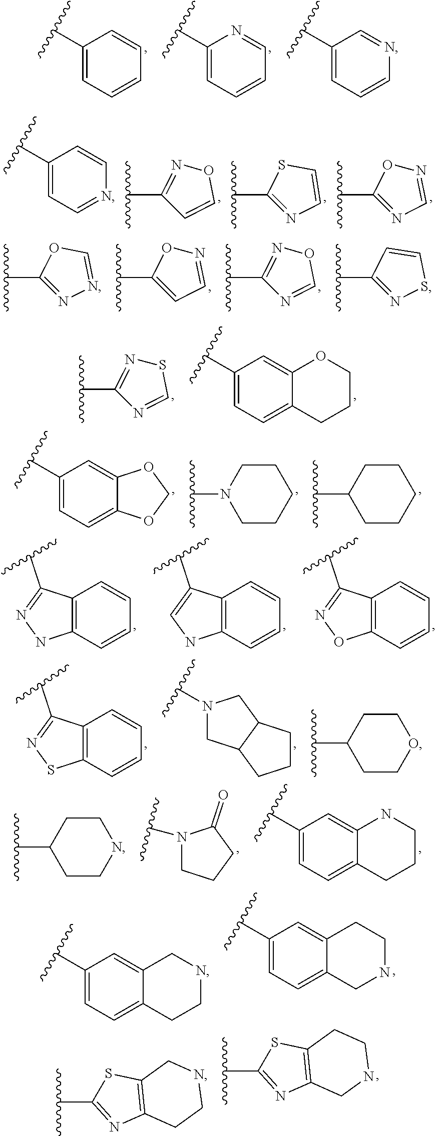

which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R² represents:

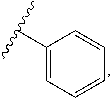 [SMILES: *c1ccccc1]

which is substituted by 0-4 substituents selected from deuterium, halogen, C₁-C₆alkyl, C₂-C₆ alkenyl, C₂-C₆ alkynyl, C₃-C₆ cycloalkyl, —OR^{a}, oxo, hydroxy (C₂-C₆ alkyl), —NR^{a}R^{b}, CR^{a}R^{b}NR^{a}R^{b}, —CN, C₁-C₆ haloalkyl, or C₁-C₆ alkoxy.

In some embodiments of the present disclosure, wherein L₂ represents —C(O)NH—; R² represents:

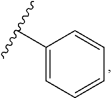 [SMILES: *c1ccccc1]

which is substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ alkoxy.

In some embodiments of the present disclosure, wherein R³ represents hydrogen or CH₃.

The present disclosure provides a compound having the structure of formula (III) or a pharmaceutically acceptable salt, stereoisomers, isotopic isomers thereof,

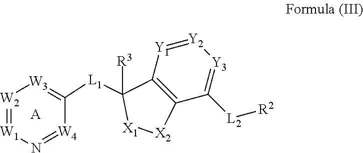

wherein, X₁ represents O, S, NR^{a}, (CR^{L}R^{L′})ₘ, C(O);
Wherein, X₂ represents O, S, NR^{a}, (CR^{T}R^{T′})ₙ, C(O);
wherein, W₁ independently represents CR^{W1} or N;
wherein, W₂ independently represents N;
wherein, W₃ independently represents CR^{W3} or N;
wherein, W₄ independently represents CR^{W4} or N;
wherein, Y₁ independently represents CR^{Y1} or N;
wherein, Y₂ independently represents CR^{Y2} or N;
wherein, Y₃ independently represents CR^{Y3} or N;
wherein, each R^{W1}, R^{W3}, R^{W4}, R^{Y1}, R^{Y2}, R^{Y3} independently represents hydrogen, deuterium, C₁-C₆alkyl, deuterated C₁-C₆alkyl, halogen, nitro, —OR^{a}, —SR^{a}, —P(O)R^{a}R^{b}, —CN, —S(O)₂R^{a}, —S(O)R^{a}, —SF₅, —NR^{a}R^{b}, —CR^{a}R^{b}NR^{a}R^{b}, —NR^{a}COR^{b}, C₁-C₆ haloalkyl, C₁-C₆ haloalkoxy, C₁-C₆ haloalkylthio, hydroxy (C₁-C₆ alkyl); or each R^{W1}, R^{W2}, R^{W3}, R^{W4}, R^{Y1}, R^{Y2}, R^{Y3} independently represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}—;
or W₁ and W₂ together with the atom which they are attached to form a 5-6 membered saturated or unsaturated ring fused to ring A, and the said ring may optionally contain 0, 1, 2 or 3 heteroatoms selected from O, N, or S;
wherein, L₁ represents absent, —(CR^{a}R^{b})ₒ—, —CR^{a}R^{b}O—, —OCR^{a}R^{b}—, —O—, —S—, —S(O)₂—, —C(O)—, —NR^{a}—, —S(O)₂NR^{a}—, —OC(O)—, —C(O)O—, —NR^{a}S(O)₂—, —CR^{a}R^{b}NR^{a}—, or —NR^{a}CR^{a}R^{b}—;
wherein, L₂ represents absent, —(CR^{a}R^{b})ₒ—, —CR^{a}R^{b}O—, —OCR^{a}R^{b}—, —O—, —C(O)—, —NR^{a}—, —S(O)₂NR^{a}—, —OC(O)—, —C(O)O—, —NR^{a}S(O)₂—, —CR^{a}R^{b}NR^{a}—, —NR^{a}CR^{a}R^{b}—, —NR^{a}C(O)NR^{b}—, —NR^{a}C(O)NR^{b}CR^{a}R^{b}—, —NR^{a}C(O)O—, —NR^{a}C(O)—, or —C(O)NR^{a}—;
wherein, R² represents C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl, —CR^{a}R^{b}C₆-C₁₀ aryl, —CR^{a}R^{b}(5-10 membered heteroaryl); which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, C₆-C₁₀ aryl, 5-10 membered heteroaryl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —S(O)₂R^{a}, —S(O)R^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, R³ represents hydrogen, halogen, C₁-C₆ haloalkyl, C₁-C₆ alkyl, or hydroxy;
or R³ and W₃ together with the atom which they are attached to form a 5-7 membered saturated or unsaturated ring fused to ring A. and the said ring may optionally contain 0, 1 or 2 heteroatoms selected from O, N, or S; and further, the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, each R^{a} and R^{b} independently represents hydrogen, deuterium, halogen, C₁-C₆ alkyl, C₁-C₆ haloalkyl, hydroxy (C₁-C₆ alkyl), C₃-C₁₀ cycloalkyl; or R^{a} and R^{b} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, each R^{L} and R^{L′} independently represents hydrogen, deuterium, halogen, C₁-C₆ alkyl, C₁-C₆ haloalkyl, hydroxy (C₁-C₆ alkyl), C₃-C₁₀ cycloalkyl; or R^{L} and R^{L′} together with the carbon atom which they are attached to form a 3-6 membered ring, and the said ring may optionally contain 0, 1 or 2 heteroatoms selected from O, N, or S; and further, the said ring may be optionally substituted by halogen, hydroxy or C₁-C₆ alkyl;
wherein, each R^{T} and R^{T′} independently represents hydrogen, deuterium, halogen, C₁-C₆ alkyl, C₁-C₆ haloalkyl, hydroxy (C₁-C₆ alkyl), C₃-C₁₀ cycloalkyl; or R^{T} and R^{T′} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, o, m, n represents the integer of 1, 2 or 3.

In some embodiments of the present disclosure, wherein W₁ represents CH, CCH₃, CF, CCF₃, CCHF₂, CCH₂F, C(OCH₃), CNH₂, CNHCH₃, CN(CH₃)₂, CCH₂NHCH₃, CCH₂N(CH₃)₂, C(CN) or N.

In some embodiments of the present disclosure, wherein W₃ represents CH, CCH₃, CF, CCF₃, CCHF₂, CCH₂F, C(OCH₃), CNH₂, CNHCH₃, C(CN) or N.

In some embodiments of the present disclosure, wherein W₄ represents CH, CCH₃, CF, CCF₃, CCHF₂, CCH₂F, C(OCH₃), CNH₂, CNHCH₃, C(CN) or N.

In some embodiments of the present disclosure, wherein Y₁ represents CH, CCH₃, CF, —CCl, CCF₃, CCHF₂, CCH₂F, C(OCH₃), C(CN) or N.

In some embodiments of the present disclosure, wherein Y₂ represents CH, CCH₃, CF, —CCl, CCF₃, CCHF₂, CCH₂F, C(OCH₃), C(CN) or N.

In some embodiments of the present disclosure, wherein Y₃ represents CH, CCH₃, CF, —CCl, CCF₃, CCHF₂, CCH₂F, C(OCH₃), C(CN) or N.

In some embodiments of the present disclosure, wherein X₁ represents —CH₂—, —CF₂—, —CHF—, —O—, —S—, —CHOH—, —CH₂CH₂— or —C(CH₃)₂—.

In some embodiments of the present disclosure, wherein X₂ represents —CH₂—, —CF₂—, —CHF—, —O—, —S—, —CHOH—, —CH₂CH₂— or —C(CH₃)₂—.

In some embodiments of the present disclosure, wherein L₁ represents —CH₂—, —CHF—, —CF₂—, —O—, —NH— or —NCH₃—.

In some embodiments of the present disclosure, wherein L₂ represents —NHC(O)—, —C(O)NH—, —NHC(O)NH—, —NHC(O)O—, —CH₂NH—, —NHCH₂—, —CH₂O—, —OCH₂— or —NH—.

In some embodiments of the present disclosure, wherein L₂ represents —NHC(O)—, —NHC(O)NH—, —NHC(O)O—, —CH₂NH—, —NHCH₂—, —CH₂O—, —OCH₂— or —NH—.

In some embodiments of the present disclosure, wherein L₂ represents —C(O)NH—.

In some embodiments of the present disclosure, wherein R² represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, phenyl, 5-6 membered heteroaryl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein L₂ represents —NHC(O)—, —NHC(O)NH—, —NHC(O)O—, —CH₂NH—, —NHCH₂—, —CH₂O—, —OCH₂— or —NH—; R² represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, phenyl, 5-6 membered heteroaryl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein R² represents C₃-C₁₀ cycloalkyl, C₆-C₁₀ cycloalkenyl, 4-10 membered heterocycloalkyl, 6-10 membered heterocycloalkenyl, C₆-C₁₀ aryl, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein L₂ represents —C(O)NH—; R² represents C₃-C₁₀ cycloalkyl, C₆-C₁₀ cycloalkenyl, 4-10 membered heterocycloalkyl, 6-10 membered heterocycloalkenyl, C₆-C₁₀ aryl, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein W₁ and W₂ combine to form a ring fused to ring A having the following structure:

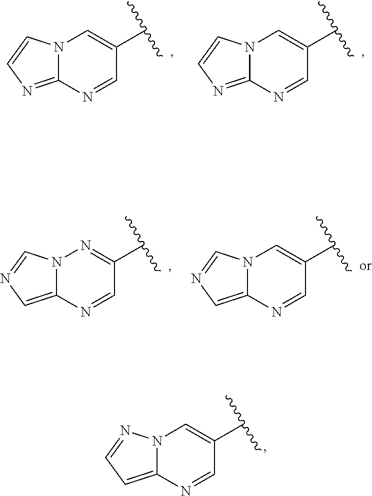 [SMILES: *c1cnc2nccn2c1 ; *c1cnc2nccn2c1 ; *c1cnc2cncn2n1 ; *c1cnc2cncn2c1 ; *c1cnc2ccnn2c1]

wherein the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)^{a}, —OC(O)NR^{a}R^{b}, —NR^{a}COR^{b} or —CONR^{a}R^{b}; and wherein the wavy line indicates the point of attachment with L₁.

In some embodiments of the present disclosure wherein W₁ and W₂ combine to form a ring fused to ring A having the following structure:

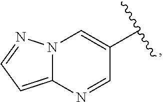 [SMILES: *c1cnc2ccnn2c1 ; ","]

wherein the wavy line indicates the point of attachment with L₁.

In some embodiments of the present disclosure, wherein R² represents phenyl, pyridinyl, indazolyl, indolyl, thiazolyl, isothiazolyl, imidazolyl, pyrazolyl, oxazolyl, isoxazolyl, 1,2,4-oxadiazolyl, 1,3,4-oxadiazolyl, pyridazinyl, piperazinyl, pyrazinyl, cyclohexyl, benzothienyl, 1,2-benzoisoxazolyl, benzo[d] isothiazolyl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, phenyl, 5-6 membered heteroaryl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein L₂ represents —NHC(O)—, —NHC(O)NH—, —NHC(O)O—, —CH₂NH—, —NHCH₂—, —CH₂O—, —OCH₂— or —NH—; R² represents phenyl, pyridinyl, indazolyl, indolyl, thiazolyl, isothiazolyl, imidazolyl, pyrazolyl, oxazolyl, isoxazolyl, 1,2,4-oxadiazolyl, 1,3,4-oxadiazolyl, pyridazinyl, piperazinyl, pyrazinyl, cyclohexyl, benzothienyl, 1,2-benzoisoxazolyl, benzo[d] isothiazolyl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, phenyl, 5-6 membered heteroaryl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R² represents

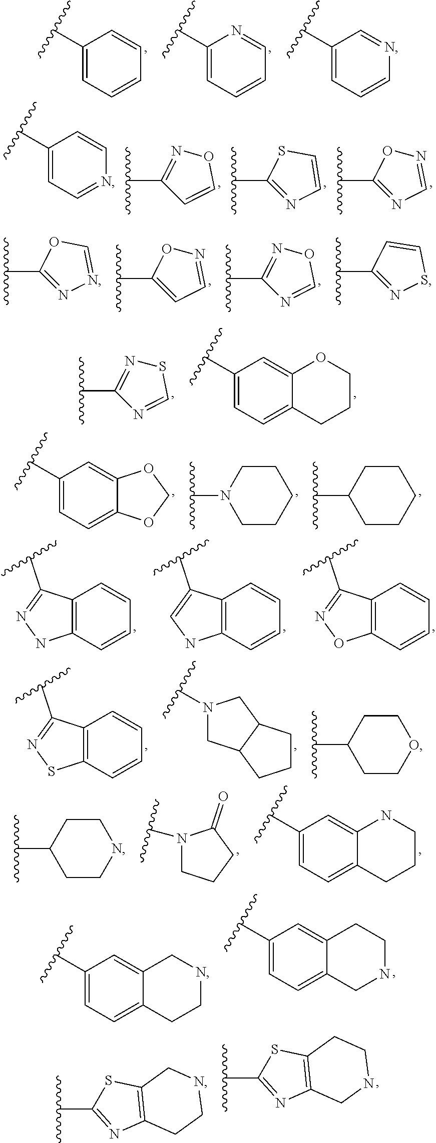

which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein L₂ represents —NHC(O)—, —NHC(O)NH—, —NHC(O)O—, —CH₂NH—, —NHCH₂—, —CH₂O—, —OCH₂— or —NH—; R² represents

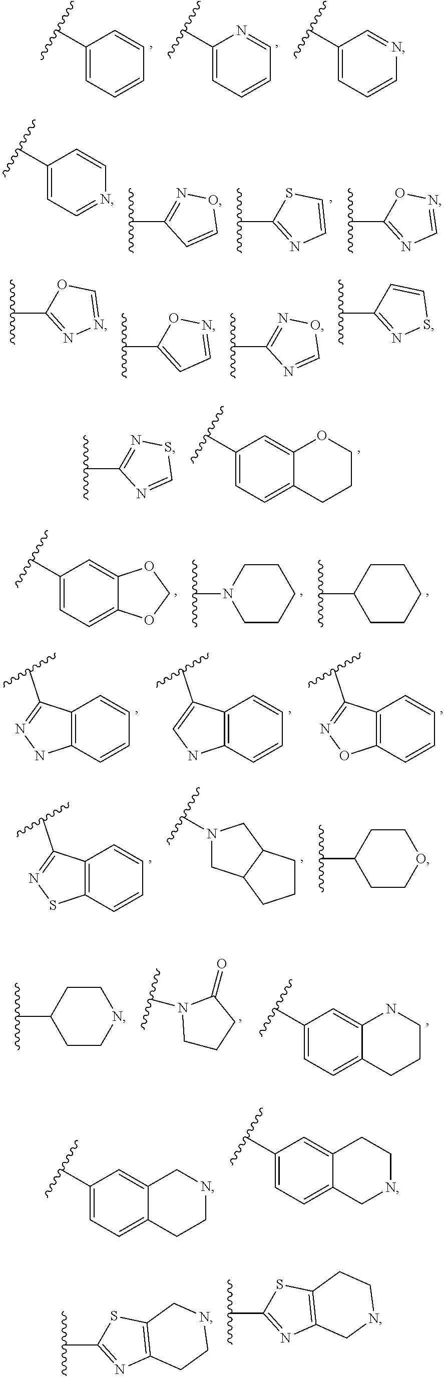

which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R² represents:

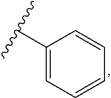 [SMILES: *c1ccccc1]

which is substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein L₂ represents —C(O)NH—; R² represents:

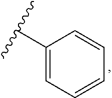 [SMILES: *c1ccccc1]

which is substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R³ represents hydrogen or CH₃.

The present disclosure provides a compound having the structure of formula (IV) or a pharmaceutically acceptable salt, stereoisomers, isotopic isomers thereof,

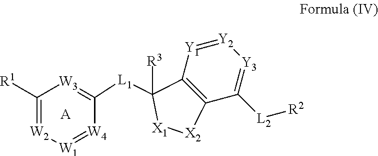

wherein, X₁ represents O, S, NR^{a}, (CR^{L}R^{L′})ₘ, C(O);
wherein, X₂ represents O, S, NR^{a}, (CR^{T}R^{T′})ₙ, C(O);
wherein, W₁ independently represents CR^{W1} or N;
wherein, W₂ independently represents CR^{W2} or N;
wherein, W₃ independently represents CR^{W3} or N;
wherein, W₄ independently represents CR^{W4} or N;
wherein, Y₁ independently represents CR^{Y1} or N;
wherein, Y₂ independently represents CR^{Y2} or N;
wherein, Y₃ independently represents CR^{Y3} or N;
wherein, each R^{W1}, R^{W2}, R^{W3}, R^{W4}, R^{Y1}, R^{Y2}, R^{Y3} independently represents hydrogen, deuterium, C₁-C₆ alkyl, deuterated C₁-C₆ alkyl, halogen, nitro, —OR^{a}, —SR^{a}, —P(O)R^{a}R^{b}, —CN, —S(O)₂R^{a}, —S(O)R^{a}, —SF₅, —NR^{a}R^{b}, —CR^{a}R^{b}NR^{a}R^{b}, —NR^{a}COR^{b}, —CONR^{a}R^{b}, C₁-C₆ haloalkyl, C₁-C₆ haloalkoxy, C₁-C₆ haloalkylthio, hydroxy (C₁-C₆alkyl); or each R^{W1}, R^{W2}, R^{W3}, R^{W4}, R^{Y1}, R^{Y2}, R^{Y3} independently represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
or W₁ and W₂ together with the atom which they are attached to form a 5-6 membered saturated or unsaturated ring fused to ring A, and the said ring may optionally contain 0, 1, 2 or 3 heteroatoms selected from O, N, or S;
or W₁ and W₄ together with the atom which they are attached to form a 5-6 membered saturated or unsaturated ring fused to ring A, and the said ring may optionally contain 0, 1, 2 or 3 heteroatoms selected from O, N, or S;
wherein, L₁ represents absent, —(CR^{a}R^{b})ₒ—, —CR^{a}R^{b}O—, —OCR^{a}R^{b}—, —O—, —S—, —S(O)₂—, —C(O)—, —NR^{a}—, —S(O)₂NR^{a}—, —OC(O)—, —C(O)O—, —NR^{a}S(O)₂—, —CR^{a}R^{b}NR^{a}—, or —NR^{a}CR^{a}R^{b}—;
wherein, L₂ represents absent, —(CR^{a}R^{b})ₒ—, —CR^{a}R^{b}O—, —OCR^{a}R^{b}—, —O—, —C(O)—, —NR^{a}—, —S(O)₂NR^{a}—, —OC(O)—, —C(O)O—, —NR^{a}S(O)₂—, —CR^{a}R^{b}NR^{a}—, —NR^{a}CR^{a}R^{b}—, —NR^{a}C(O)NR^{b}—, —NR^{a}C(O)NR^{b}CR^{a}R^{b}—, —NR^{a}C(O)O—, —NR^{a}C(O)—, or —C(O)NR^{a}—.

Wherein, R¹ represents hydrogen, deuterium, C₁-C₆ alkyl, deuterated C₁-C₆ alkyl, halogen, —OR^{a}, —SR^{a}, —P(O)R^{a}R^{b}, —CN, —S(O)₂R^{a}, —S(O)R^{a}, —SF₅, —NR^{a}R^{b}, C₁-C₆ haloalkyl, C₁-C₆ haloalkoxy, C₁-C₆ haloalkylthio, hydroxy (C₁-C₆ alkyl); or R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl, which are substituted by 0-4 substituents that selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
or R¹ and W₂ together with the atom which they are attached to form a 5-6 membered saturated or unsaturated ring fused to ring A. and the said ring may optionally contain 0, 1 or 2 heteroatoms selected from O, N, or S; and further, the said ring may optionally substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, R² represents C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl; which are substituted by 0-4 substituents selected from deuterium, halogen, C₁-C₆ alkyl, C₂-C₆ alkenyl, C₂-C₆alkynyl, C₃-C₆cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —S(O)₂R^{a}, —S(O)R^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, R³ represents hydrogen, halogen, C₁-C₆ haloalkyl, C₁-C₆ alkyl, or hydroxy;
or R³ and W₃ together with the atom which they are attached to form a 5-7 membered saturated or unsaturated ring fused to ring A, and the said ring may optionally contain 0, 1 or 2 heteroatoms selected from O, N, or S; and further, the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, each R^{a} and R^{b} independently represents hydrogen, C₁-C₆ alkyl, C₁-C₆ haloalkyl, hydroxy (C₁-C₆ alkyl), C₃-C₁₀ cycloalkyl; or R^{a} and R^{b} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, each R^{L} and R^{L′} independently represents hydrogen, C₁-C₆ alkyl, C₁-C₆ haloalkyl, hydroxy (C₁-C₆ alkyl), C₃-C₁₀ cycloalkyl; or R^{L} and R^{L′} together with the carbon atom which they are attached to form a 3-6 membered ring, and the said ring may optionally contain 0, 1 or 2 heteroatoms selected from O, N, or S; and further, the said ring may be optionally substituted by halogen, hydroxy or C₁-C₆ alkyl;
wherein, each R^{T} and R^{T′} independently represents hydrogen, C₁-C₆ alkyl, C₁-C₆ haloalkyl, hydroxy (C₁-C₆ alkyl), C₃-C₁₀ cycloalkyl; or R^{T} and R^{T′} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, o, m, n represents the integer of 1, 2 or 3.

In some embodiments of the present disclosure, wherein W₁ represents CH, CCH₃, CF, CCF₃, CCHF₂, CHF₂, COCH₃, C(CN) or N.

In some embodiments of the present disclosure, wherein W₁ represents N.

In some embodiments of the present disclosure, wherein W₂ represents CH, CCH₃, CF, CCF₃, CCHF₂, CHF₂, COCH₃, CNH₂, CNHCH₃, CN(CH₃)₂, CH₂NCH₃, CH₂N(CH₃)₂, C(CN) or N.

In some embodiments of the present disclosure, wherein W₃ represents CH, CCH₃, CF, CCF₃, CCHF₂, CHF₂, COCH₃, C(CN) or N.

In some embodiments of the present disclosure, wherein W₄ represents CH, CCH₃, CF, CCF₃, CCHF₂, CHF₂, COCH₃, CNH₂, CNHCH₃, C(CN) or N.

In some embodiments of the present disclosure, wherein Y₁ represents CH, CCH₃, CF, CCl, CCF₃, CCHF₂, CHF₂, COCH₃, C(CN) or N.

In some embodiments of the present disclosure, wherein Y₂ represents CH, CCH₃, CF, CCl, CCF₃, CCHF₂, CHF₂, COCH₃, C(CN) or N.

In some embodiments of the present disclosure, wherein Y₃ represents CH, CCH₃, CF, CCl, CCF₃, CCHF₂, CHF₂, COCH₃, C(CN) or N.

In some embodiments of the present disclosure, wherein X₁ represents CH₂, CF₂, CHF, O, S, CHOH, CH₂CH₂ or C(CH₃)₂.

In some embodiments of the present disclosure, wherein X₂ represents CH₂, CF₂, CHF, O, S, CHOH, CH₂CH₂ or C(CH₃)₂.

In some embodiments of the present disclosure, wherein L₁ represents CH₂, CF₂, O, NH or NCH₃.

In some embodiments of the present disclosure, wherein L₂ represents —NHC(O)—, —C(O)NH—, —NHC(O)NH—, —NHC(O)O—, —CH₂NH—, —NHCH₂—, —CH₂O—, —OCH₂— or —NH—.

In some embodiments of the present disclosure, wherein R¹ represents C₁-C₆ alkyl, halogen, —OH, O(C₁-C₆ alkyl); or R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substitutes selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substitutes selected from deuterium, halogen, C₁-C₆ alkyl, —OR^{a}, hydroxy (C₁-C₆ alkyl), —CN, C₁-C₆ haloalkyl.

In some embodiments of the present disclosure, wherein R¹ represents 5-10 membered heteroaryls, which are substituted by 0-4 substitutes selected from deuterium, halogen, C₁-C₆ alkyl, —OR^{a}, hydroxy (C₁-C₆ alkyl), —CN, C₁-C₆ haloalkyl.

In some embodiments of the present disclosure, wherein R¹ represents pyrazolyl, triazolyl, oxazolyl, thiazolyl, imidazolyl, which are substituted by 0-4 substitutes selected from deuterium, halogen, C₁-C₆ alkyl, —OR^{a}, hydroxy (C₁-C₆ alkyl), —CN, C₁-C₆ haloalkyl.

In some embodiments of the present disclosure, wherein R¹ represents

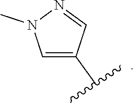 [SMILES: *c1cnn(C)c1]

In some embodiments of the present disclosure, wherein R² represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryls, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein R¹ and W₂ combine to form a ring fused to ring A having the following structure:

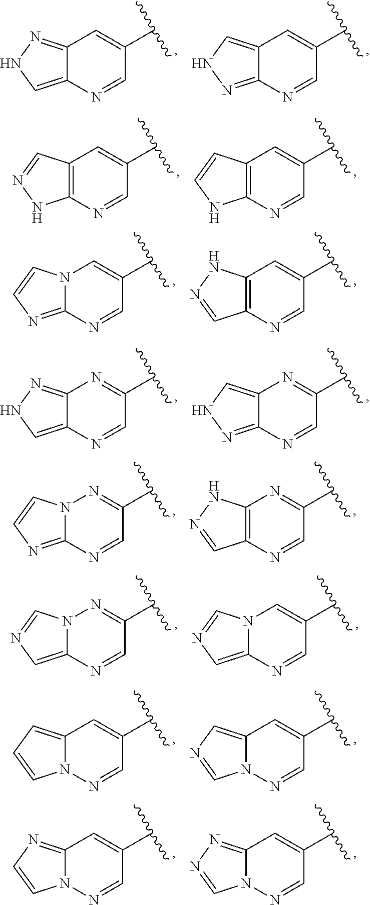

wherein the said ring may optionally be substituted by 0, 1 or 2 substituents selected from deuterium, halogen, C₁-C₆ alkyl, C₂-C₆ alkenyl, C₂-C₆ alkynvl, C₃-C₆ cycloalkyl, —OR^{a}, oxo, hydroxy (C₁-C₆ alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}; and wherein the wavy line indicates the point of attachment with L₁.

In some embodiments of the present disclosure, wherein R¹ and W₂ combine to form a ring fused to ring A having the following structure:

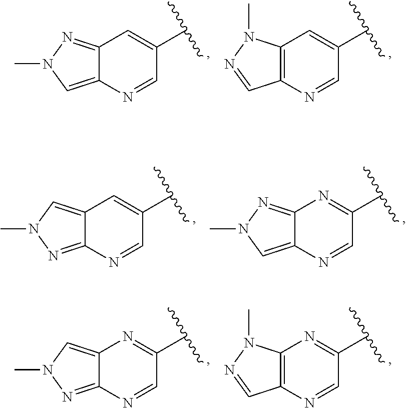 [SMILES: *c1cnc2cn(C)nc2c1 ; *c1cnc2cnn(C)c2c1 ; *c1cnc2nn(C)cc2c1 ; *c1cnc2cn(C)nc2n1 ; *c1cnc2nn(C)cc2n1 ; *c1cnc2cnn(C)c2n1]

wherein the wavy line indicates the point of attachment with L₁.

In some embodiments of the present disclosure, wherein R² represents phenyl, pyridinyl, indazolyl, indolyl, thiazolyl, isothiazolyl, imidazolyl, pyrazolyl, oxazolyl, isoxazolyl, 1,2,4-oxadiazolyl, 1,3,4-oxadiazolyl, pyridazinyl, piperazinyl, pyrazinyl, cyclohexyl, benzothienyl, 1,2-benzoisoxazolyl, benzo[d] isothiazolyl, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R² represents:

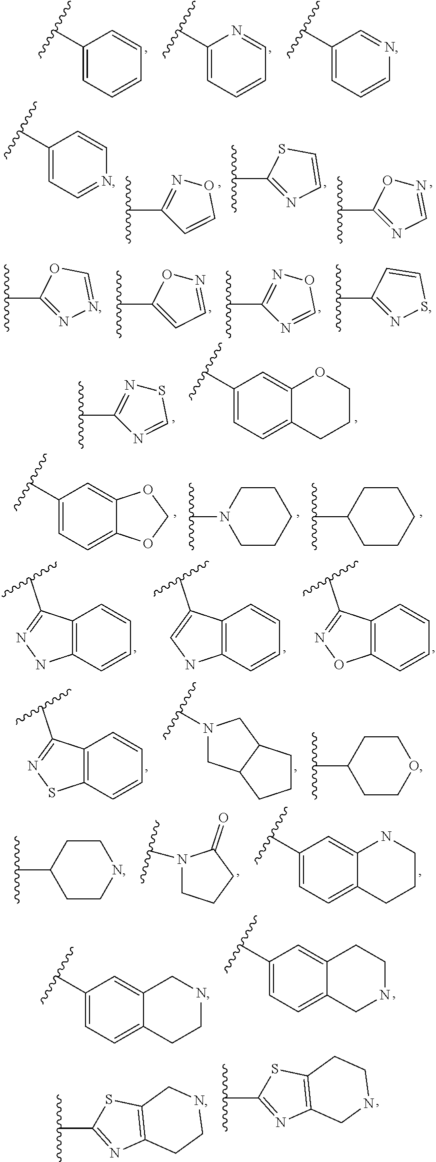

which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R² represents:

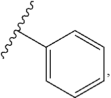 [SMILES: *c1ccccc1]

which is substituted by 0-4 substituents selected from deuterium, halogen, C₁-C₆ alkyl, C₂-C₆ alkenyl, C₂-C₆alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R³ represents hydrogen or CH₃.

Specifically, the present disclosure provides compounds having the following structures:

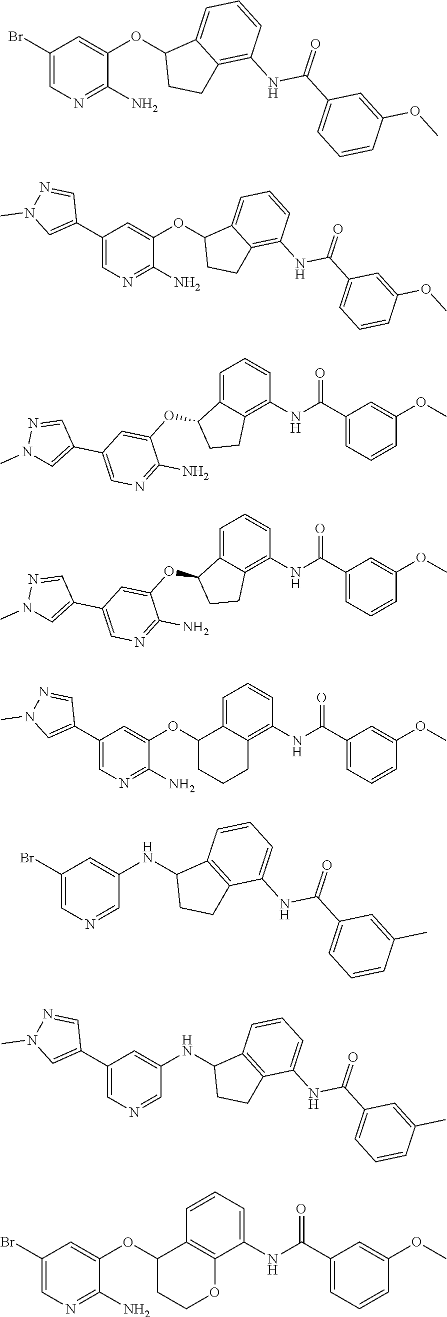

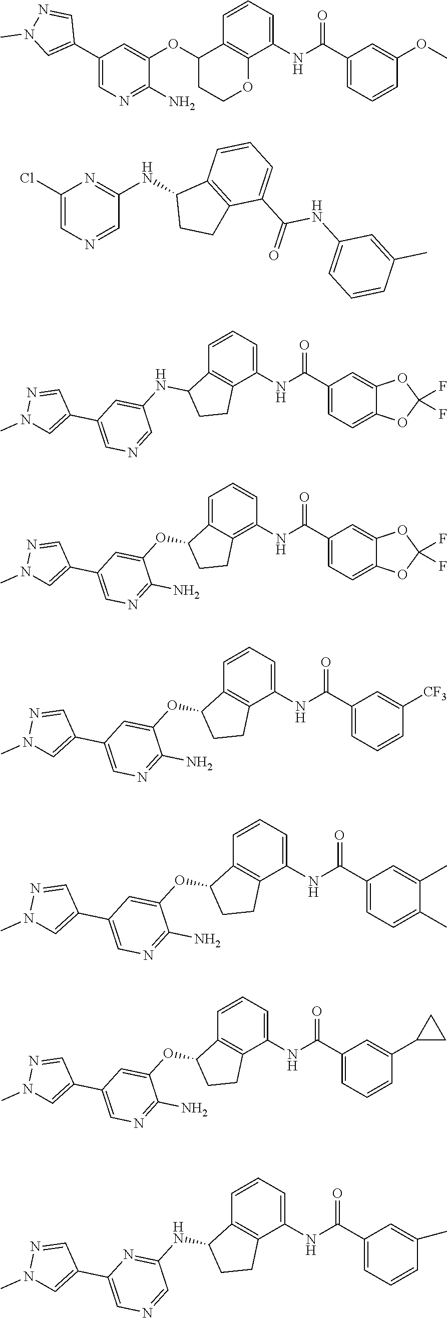

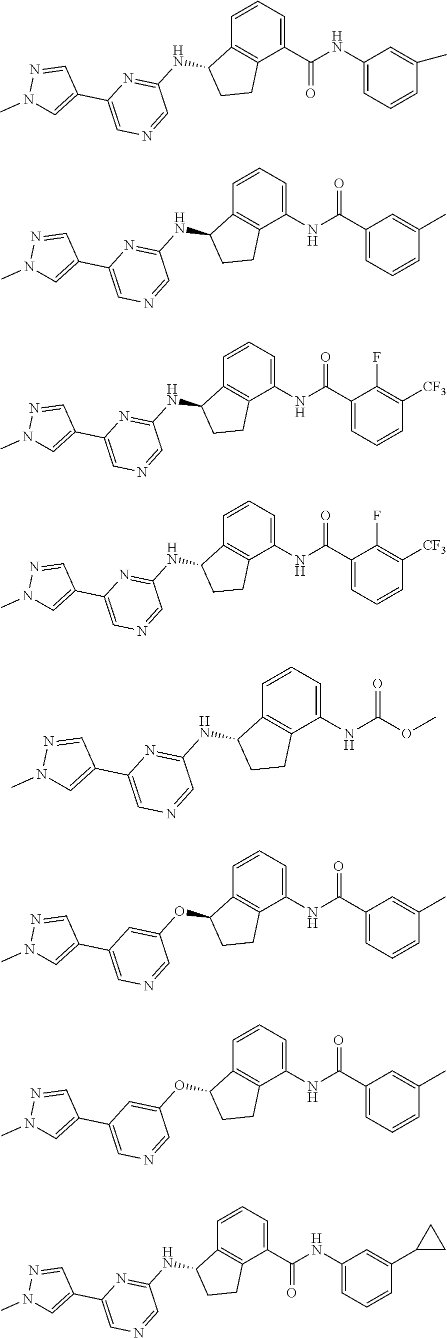

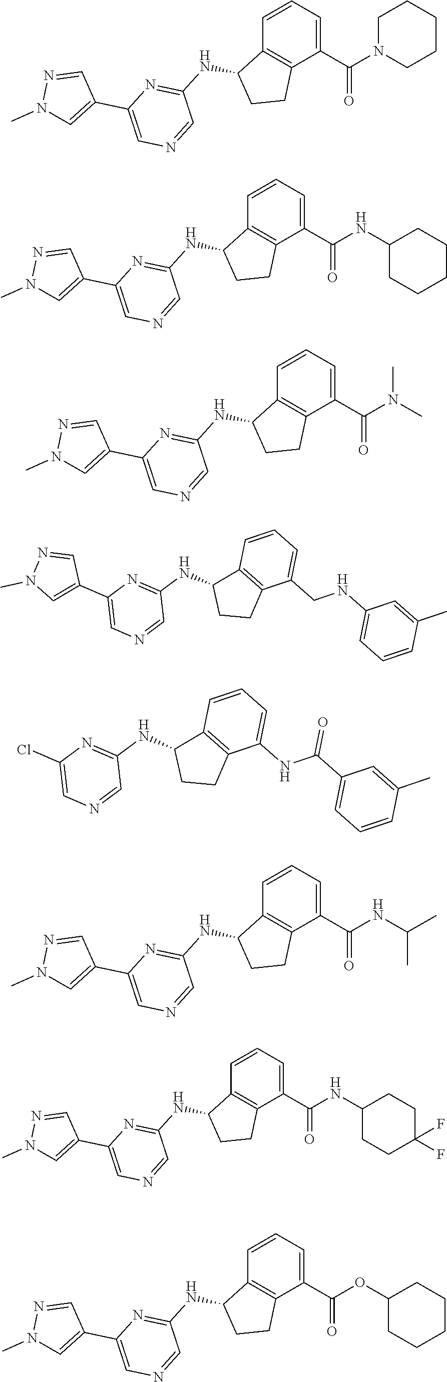

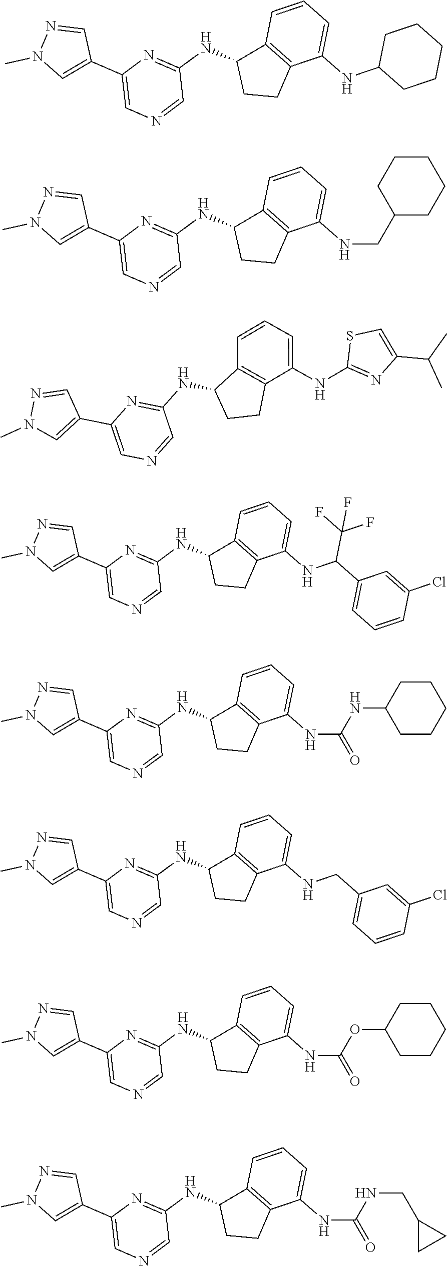

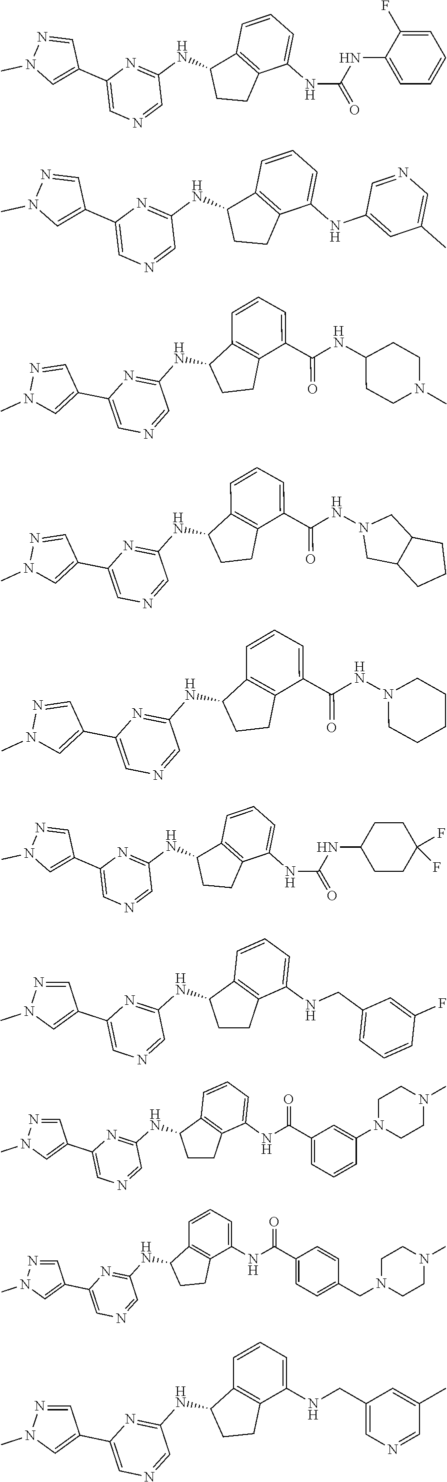

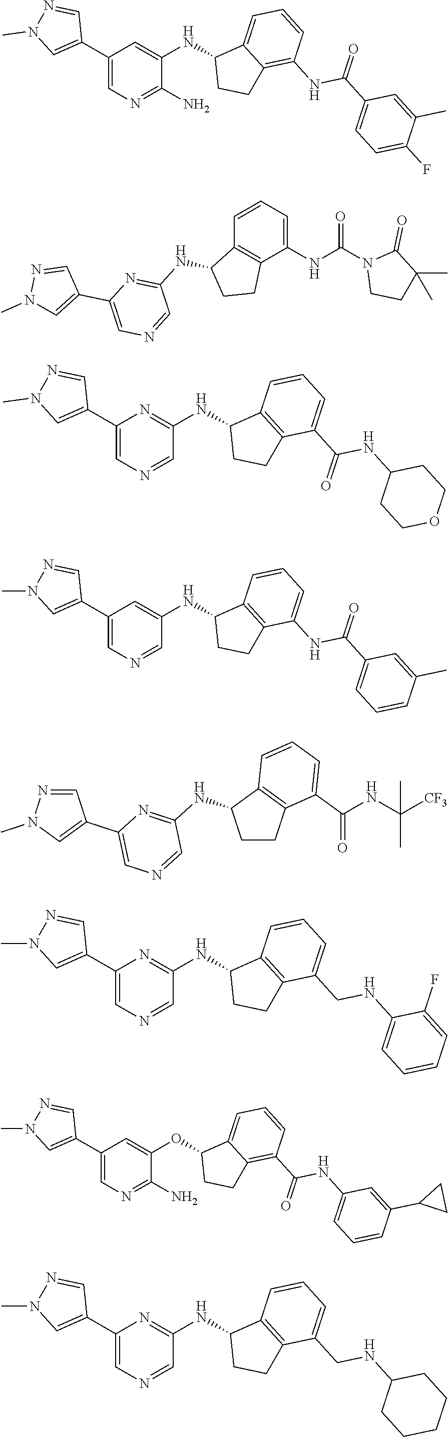

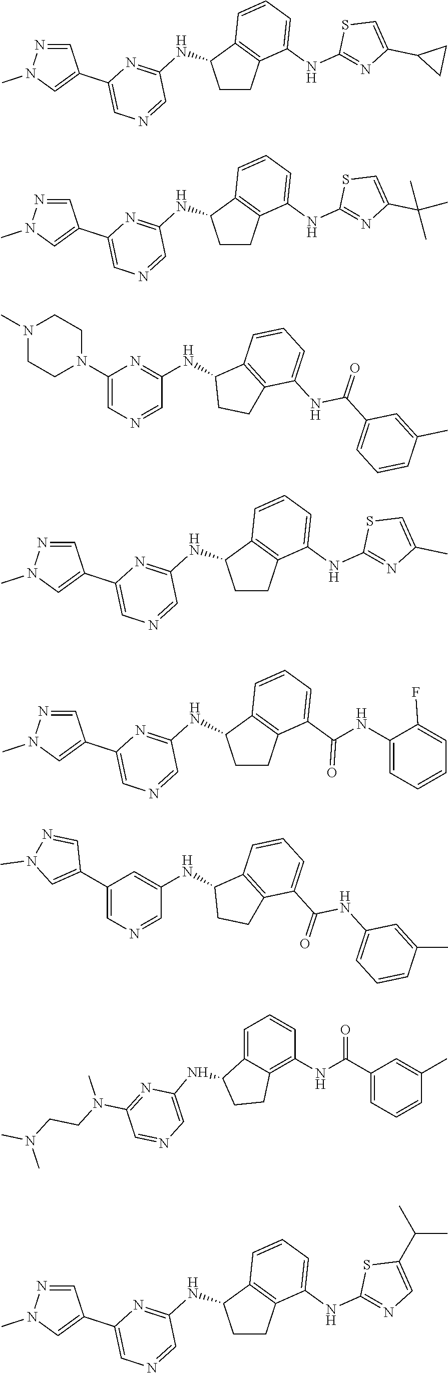

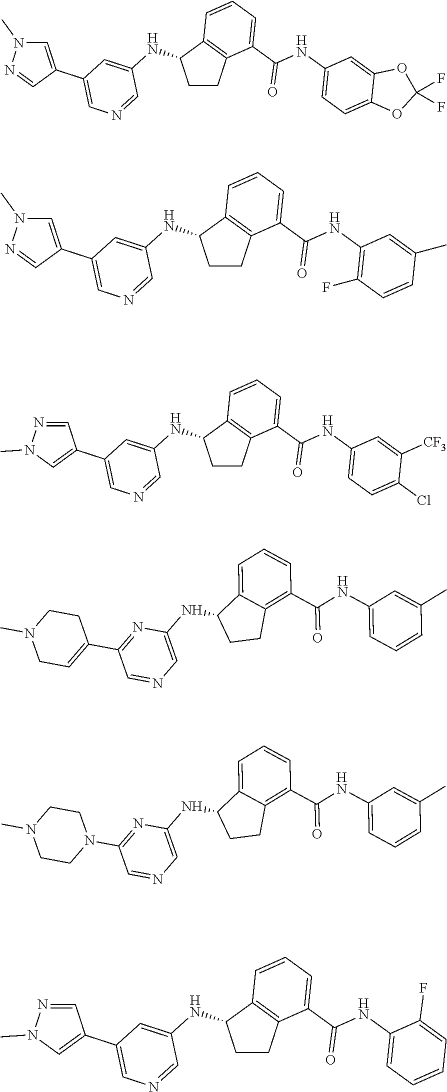

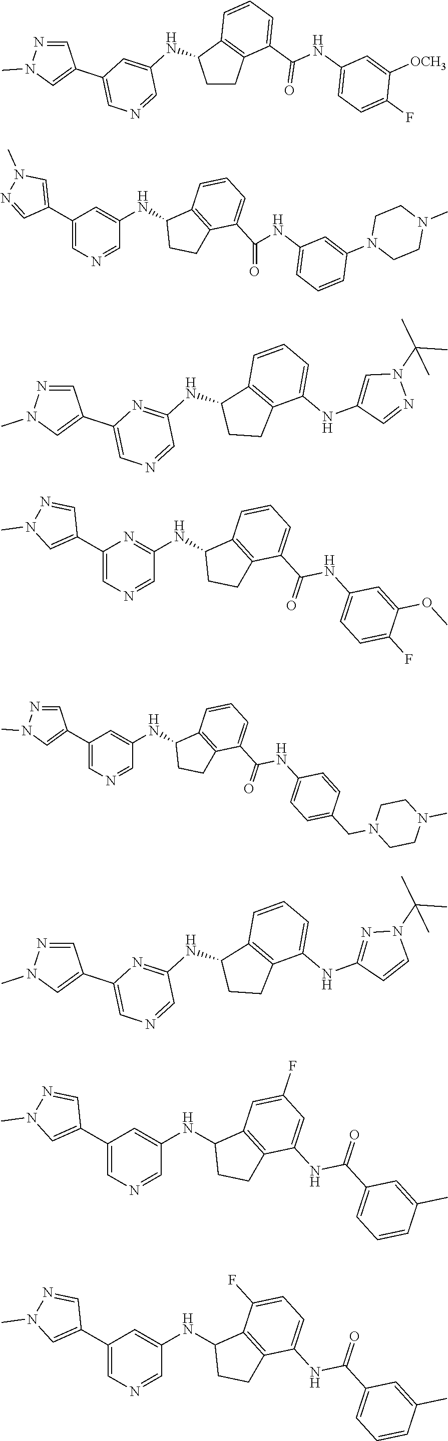

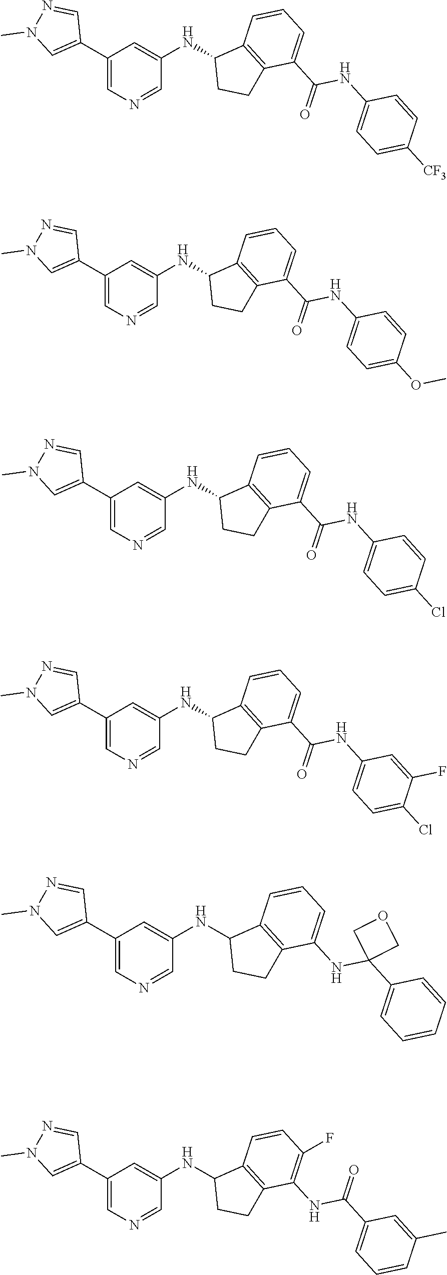

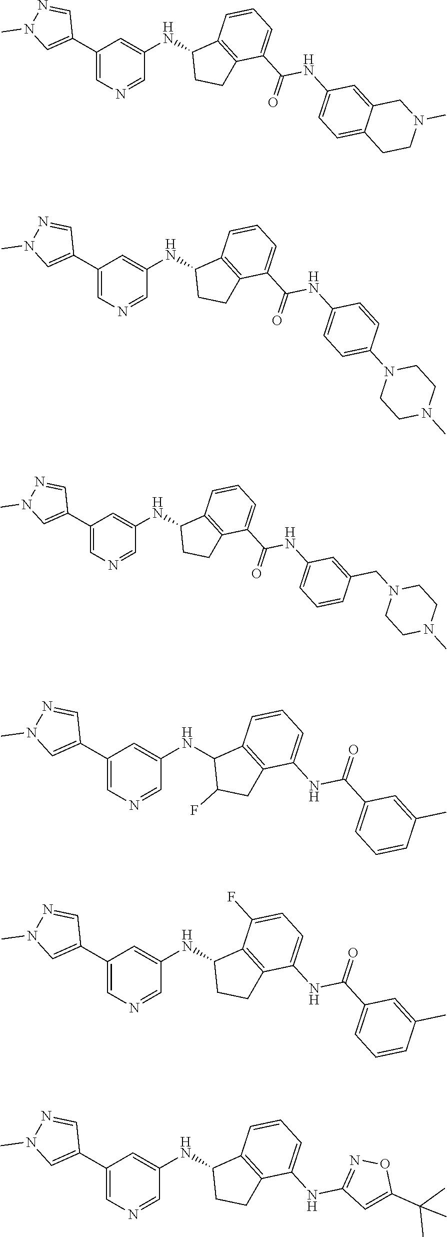

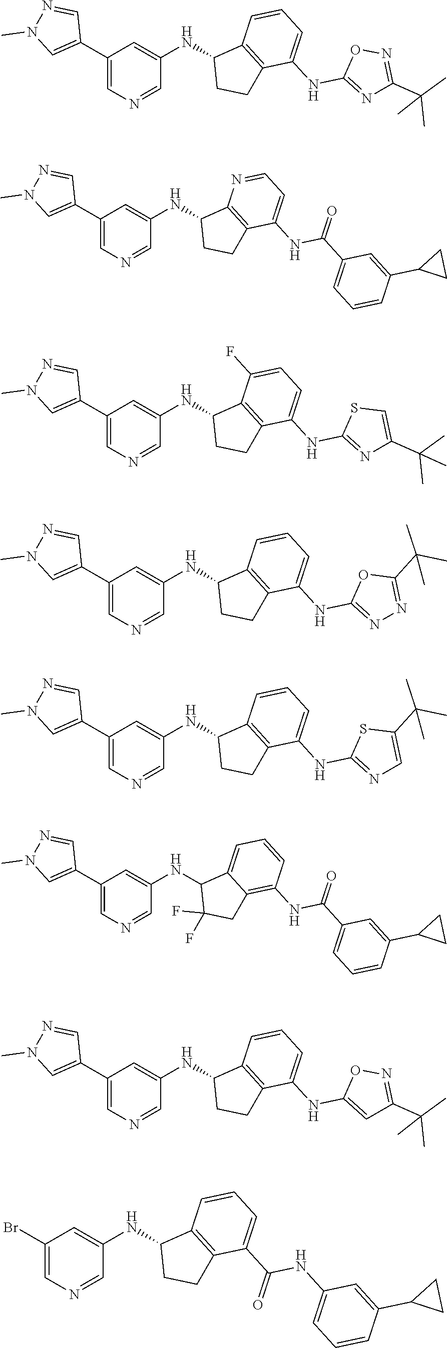

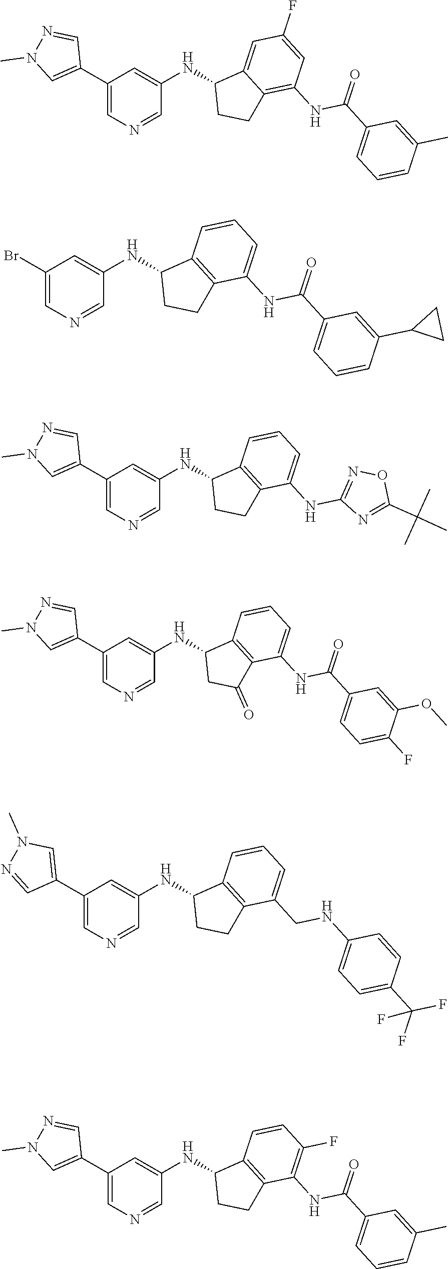

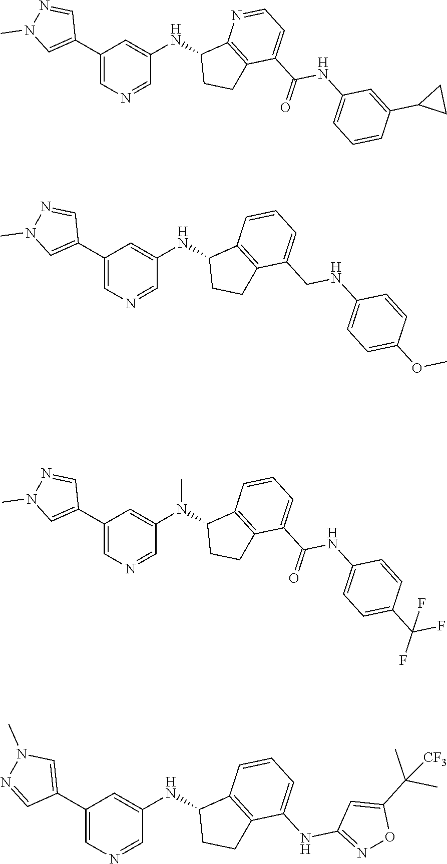

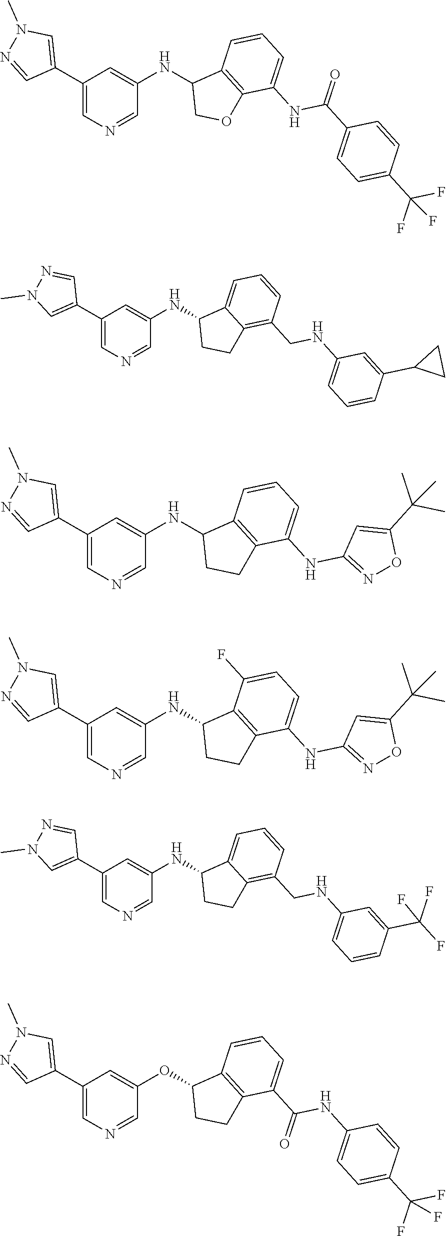

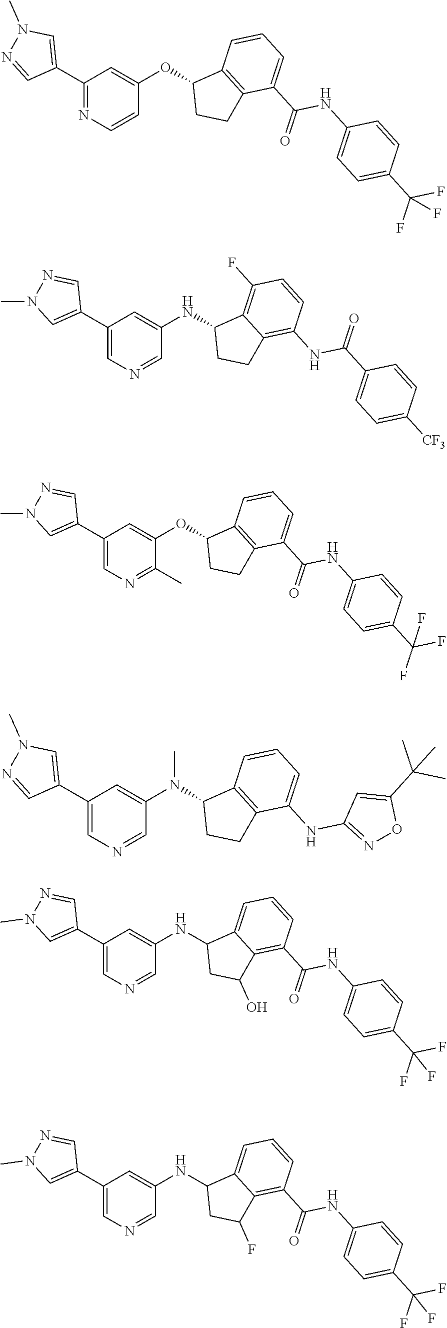

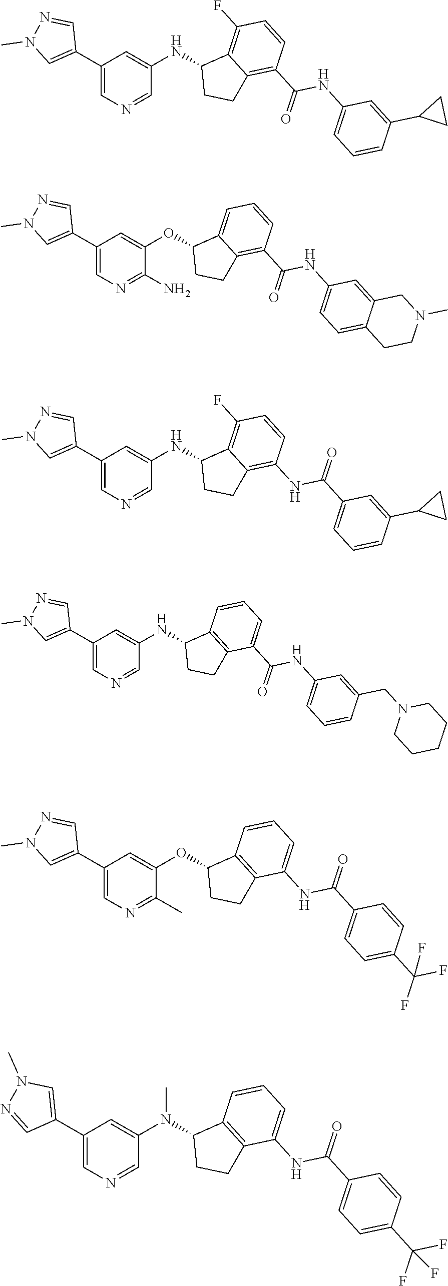

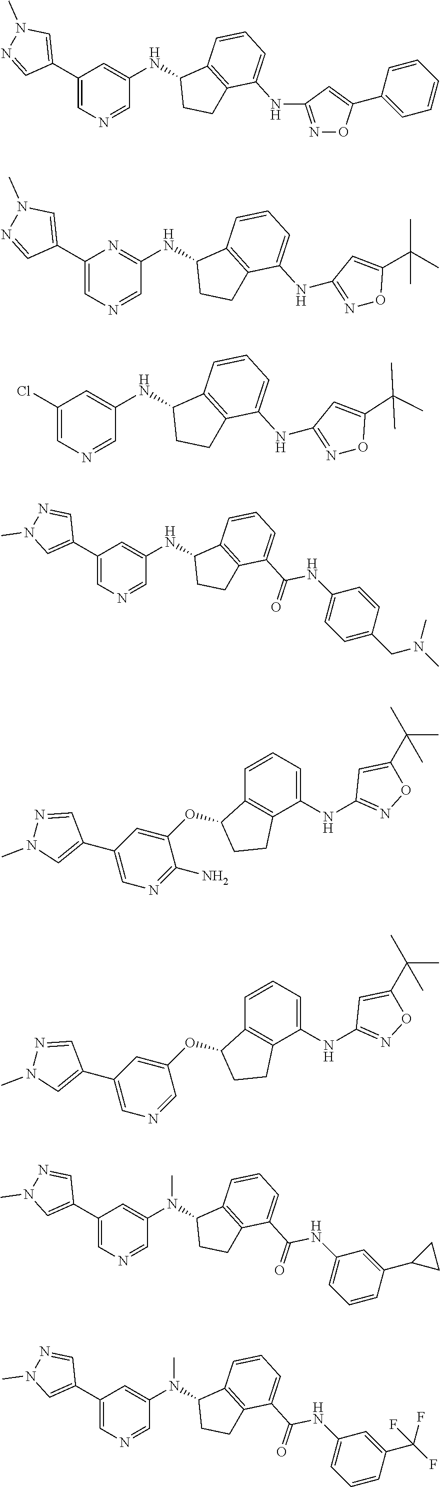

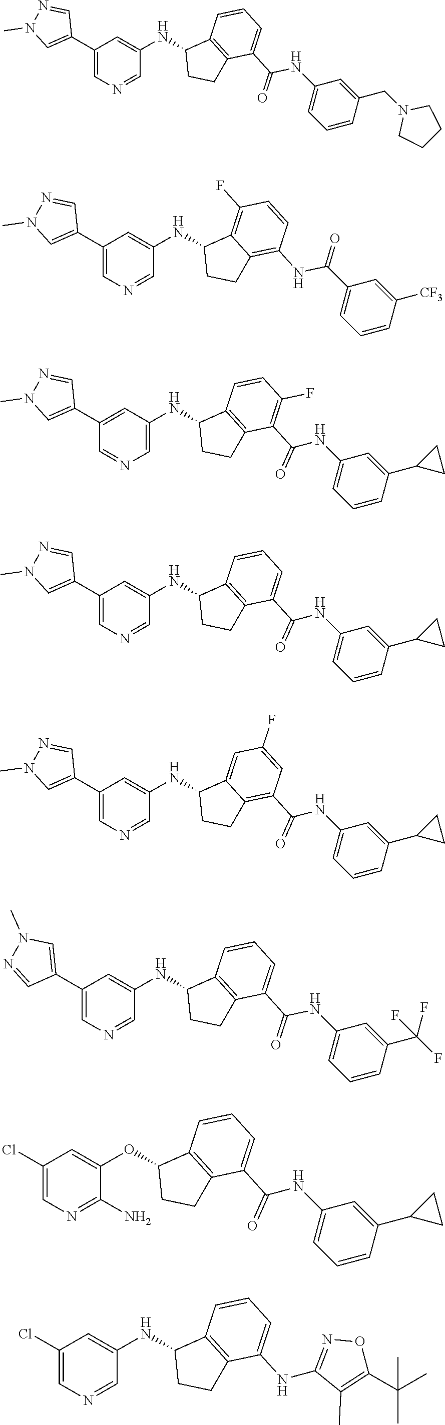

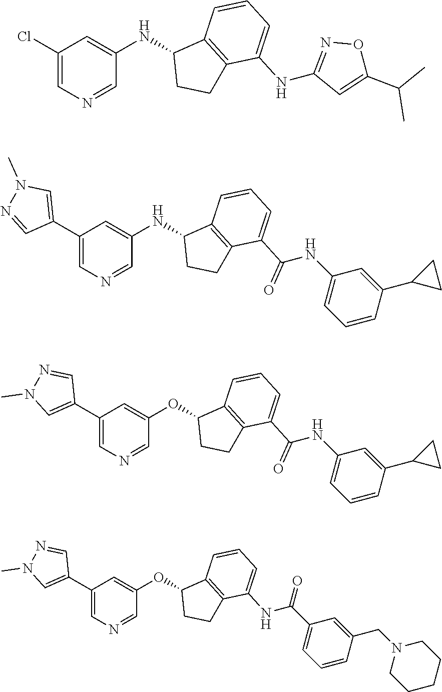

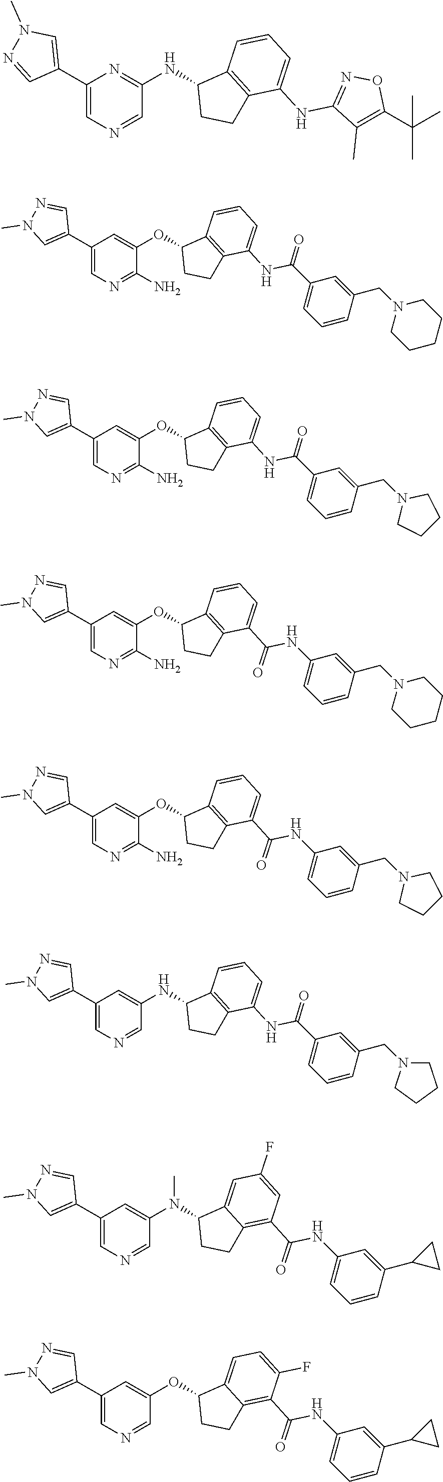

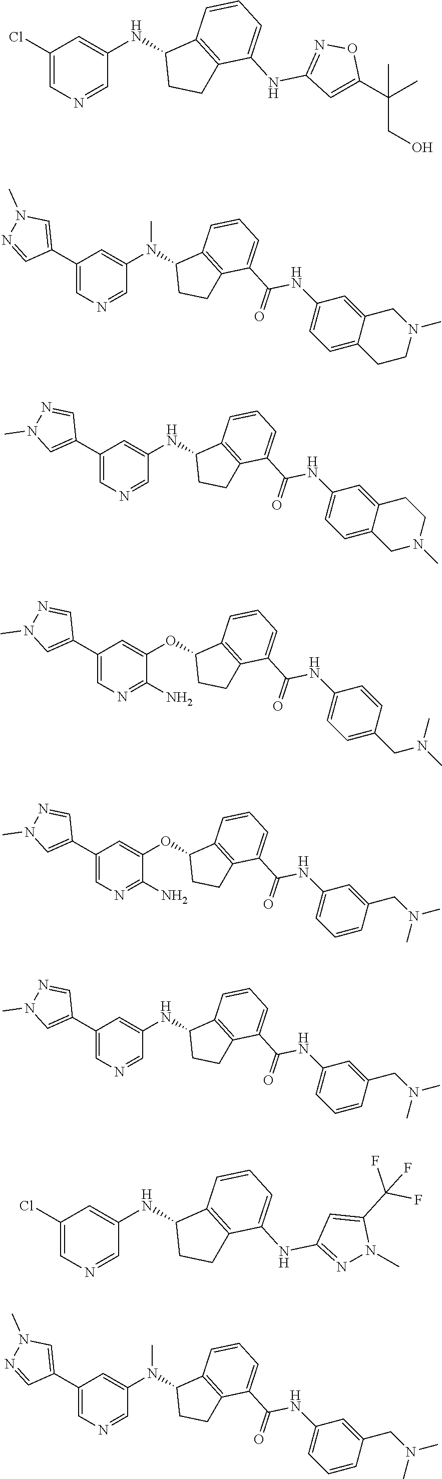

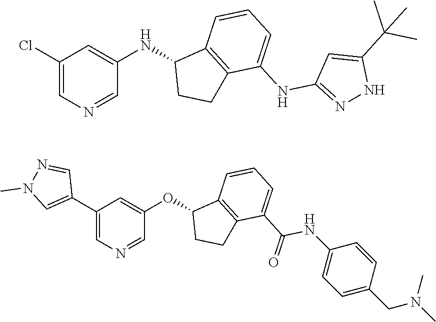

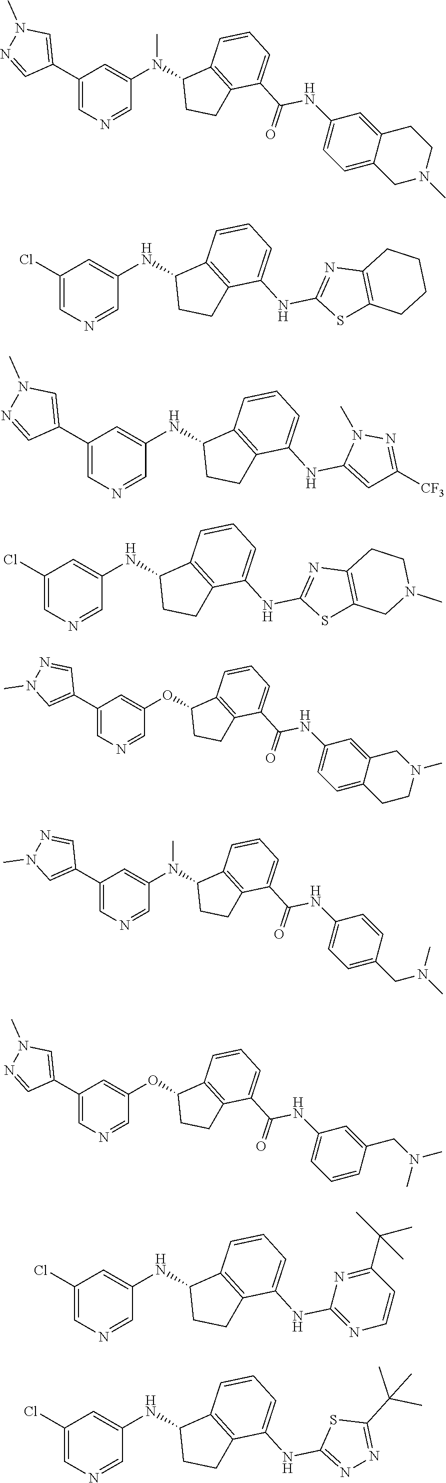

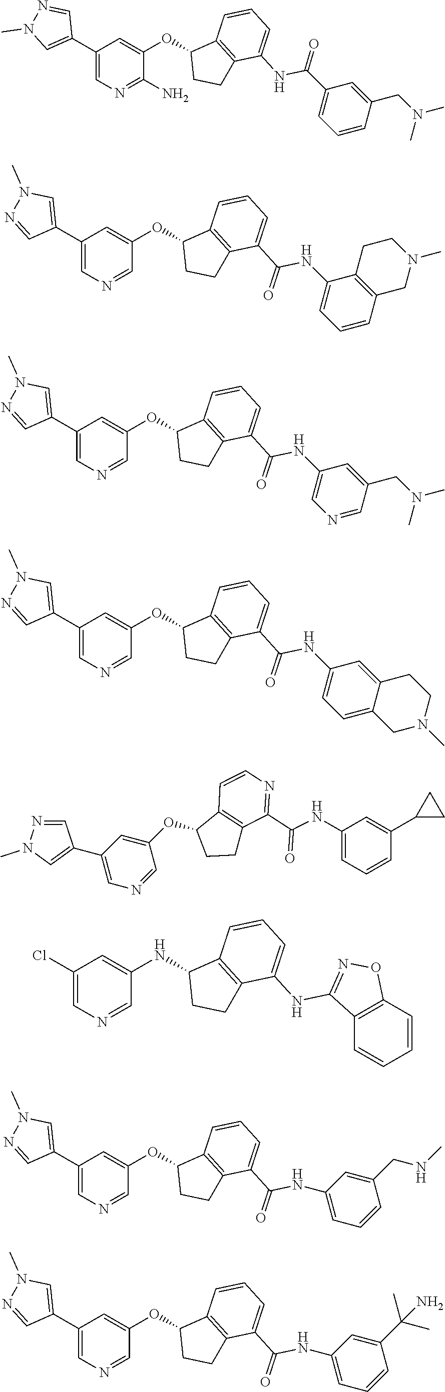

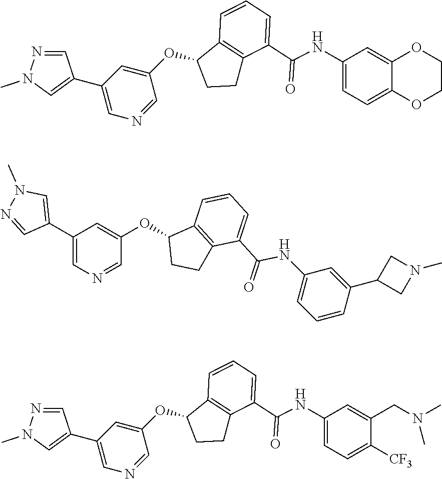

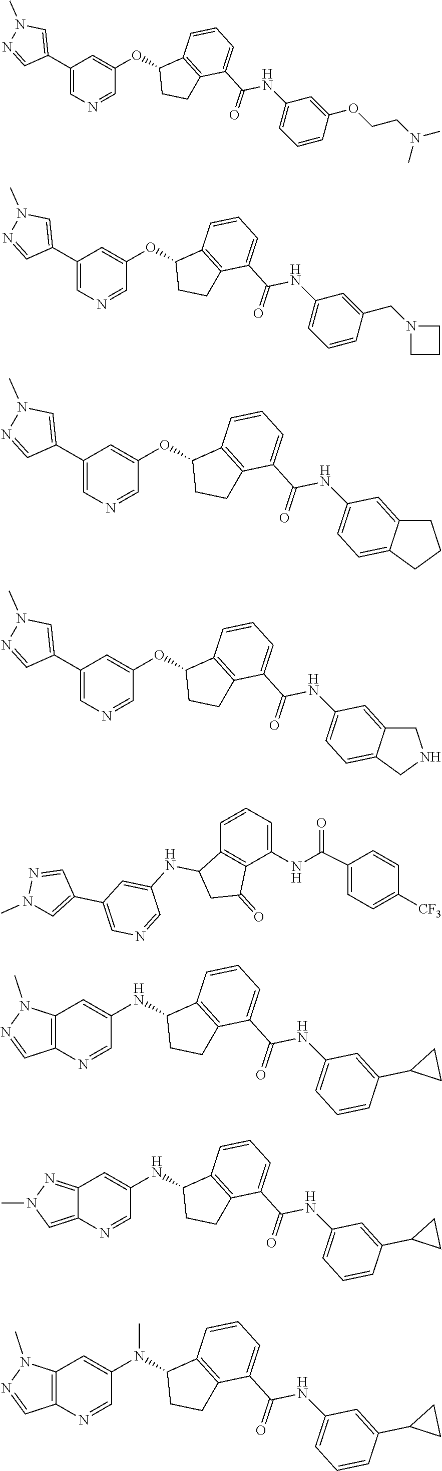

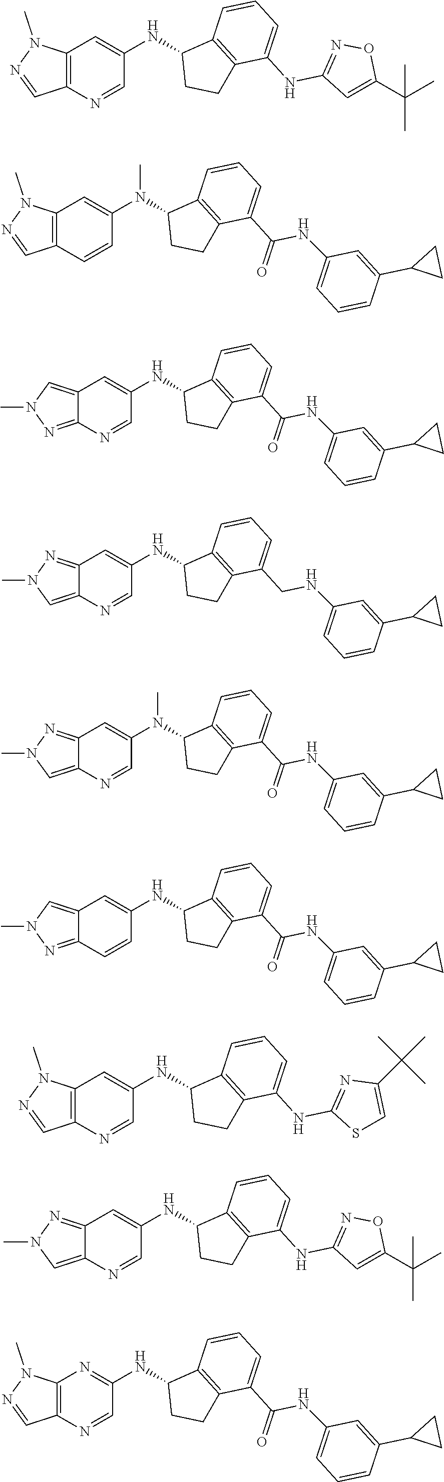

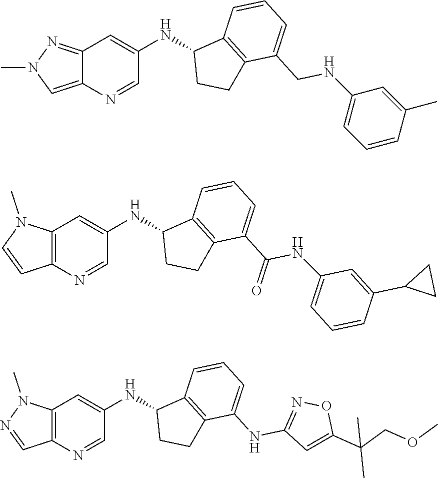

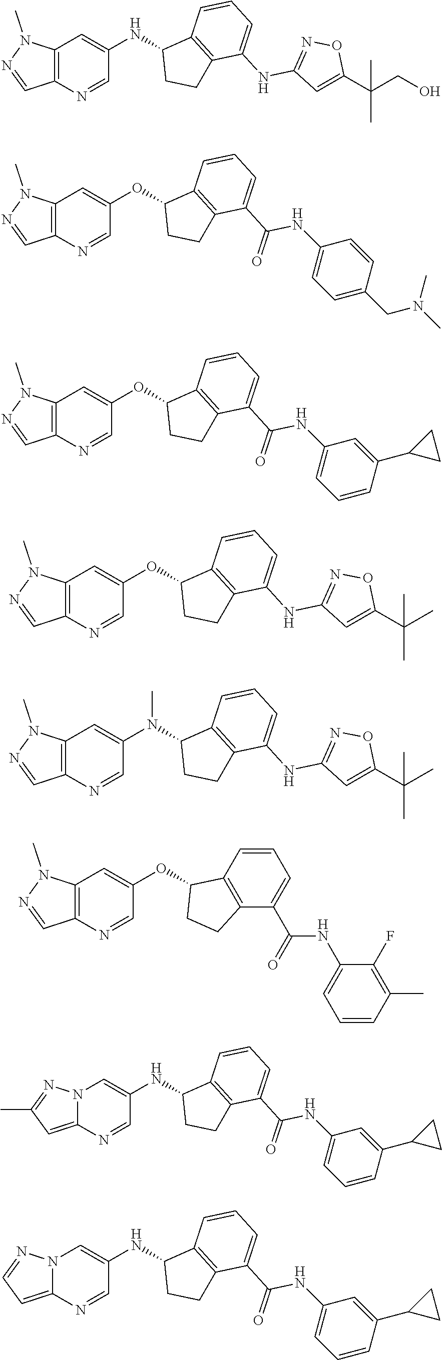

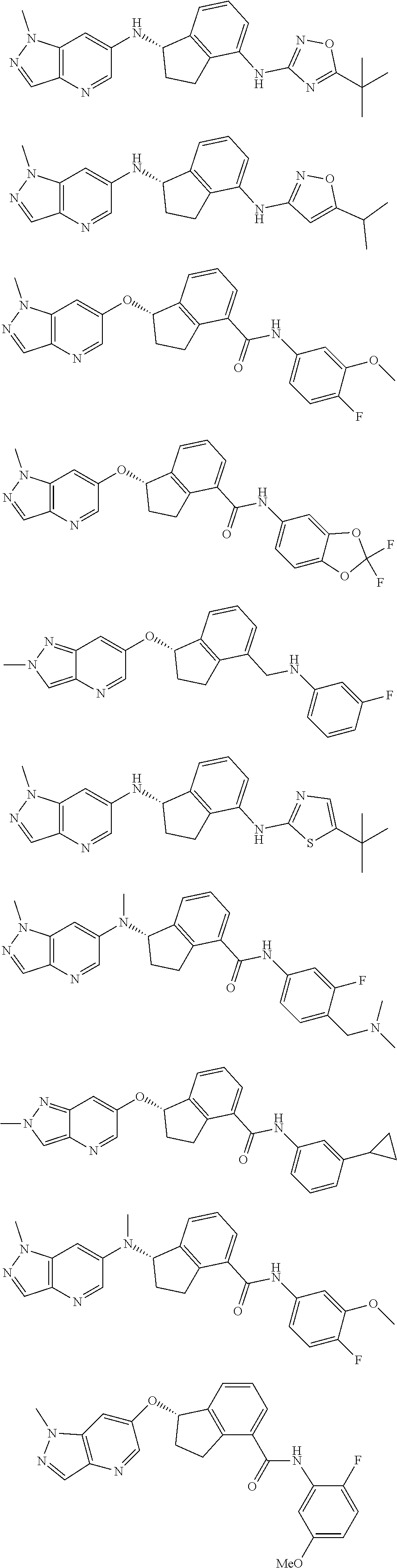

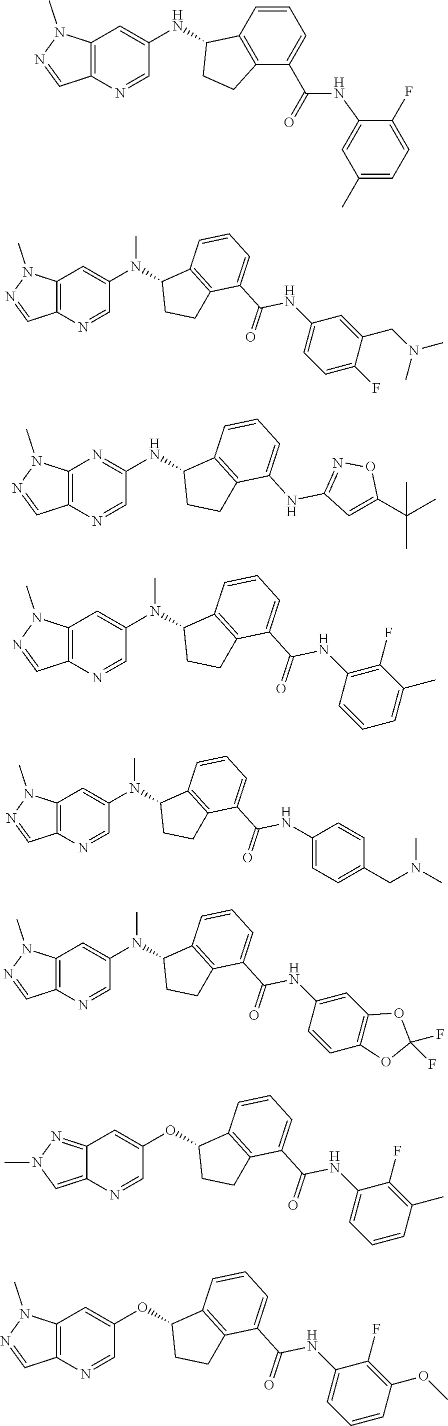

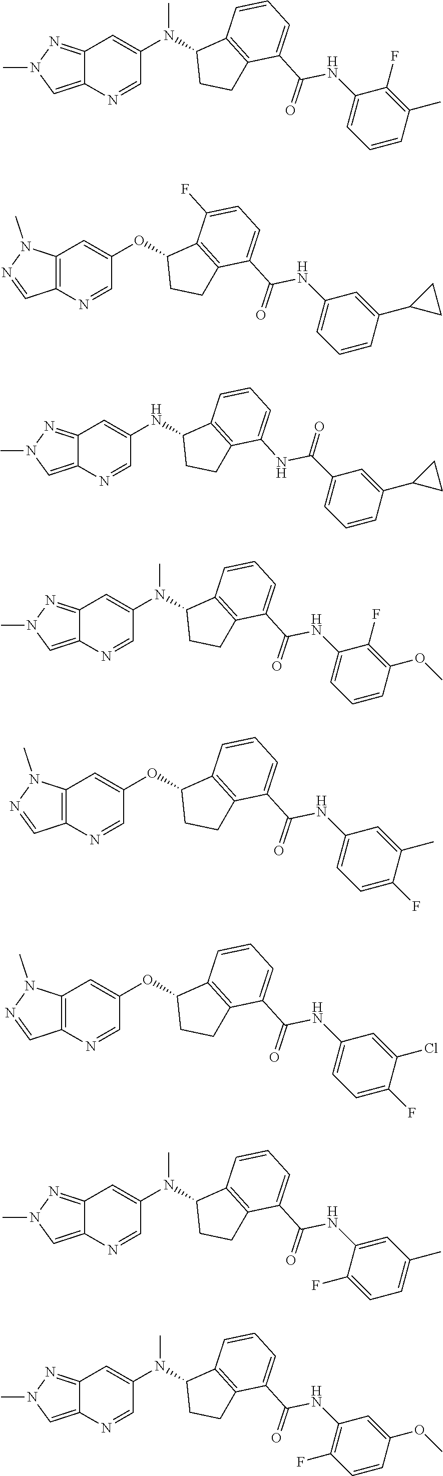

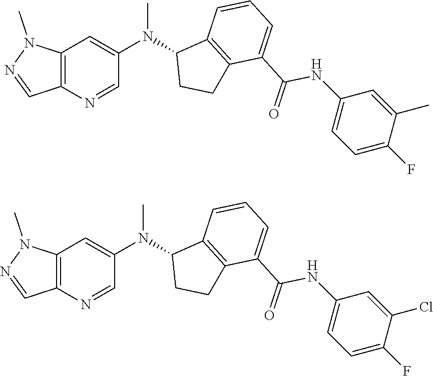 [SMILES: Cc1cc(NC(=O)c2cccc3c2CC[C@@H]3N(C)c2cnc3cnn(C)c3c2)ccc1F ; CN(c1cnc2cnn(C)c2c1)[C@H]1CCc2c(C(=O)Nc3ccc(F)c(Cl)c3)cccc21]

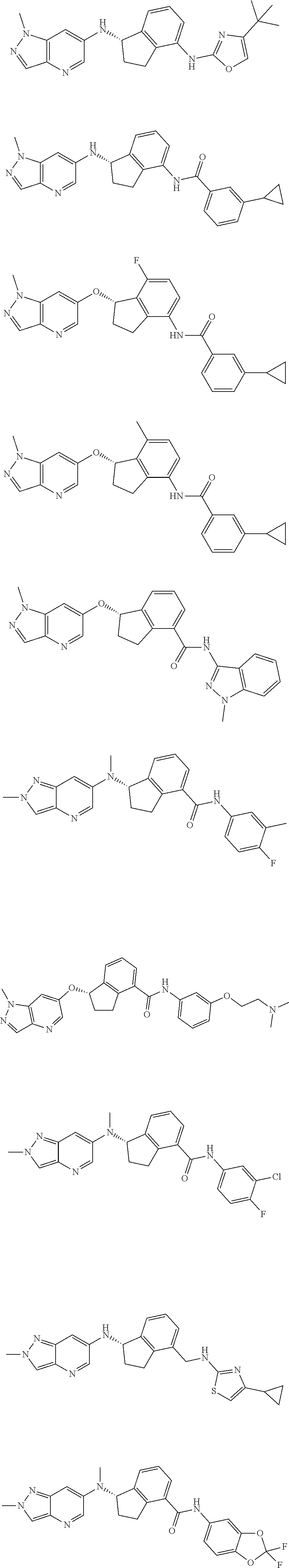

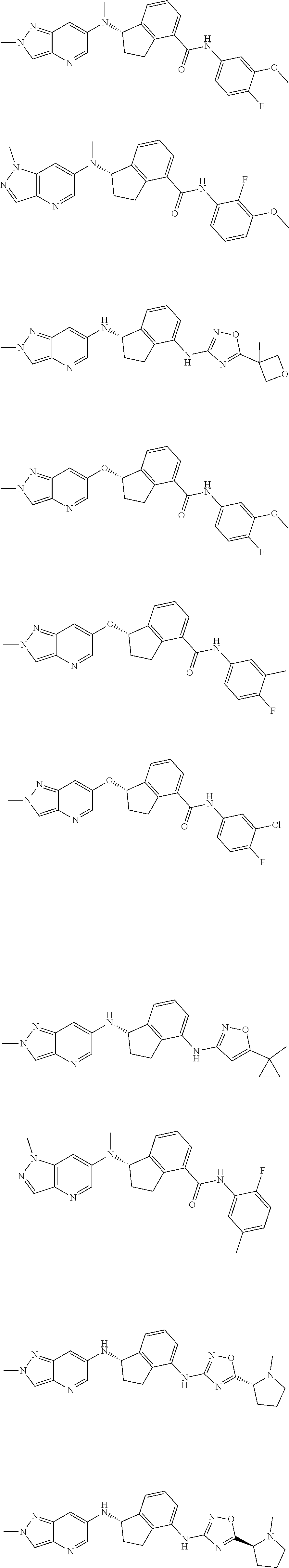

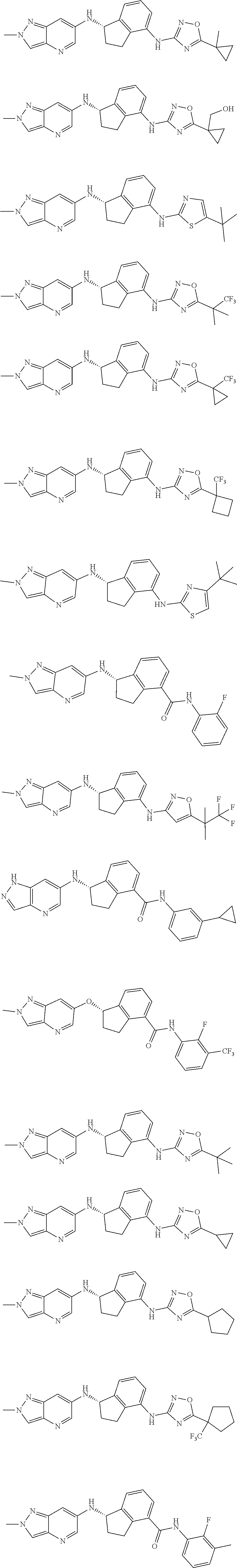

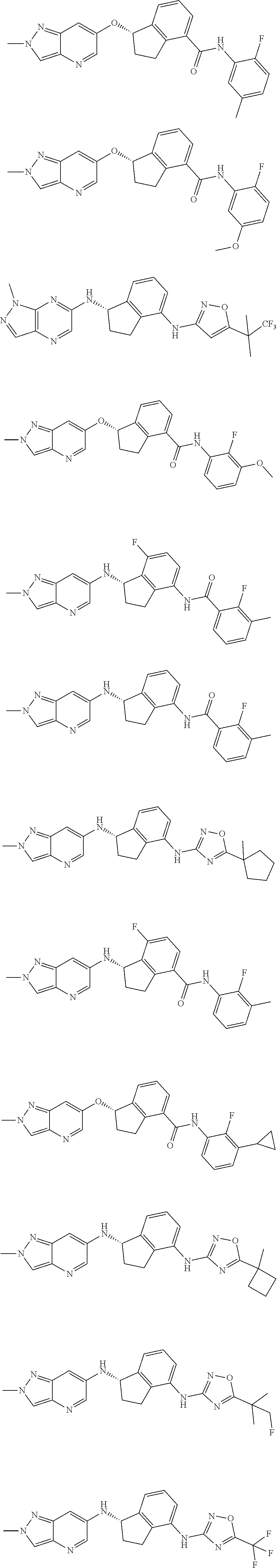

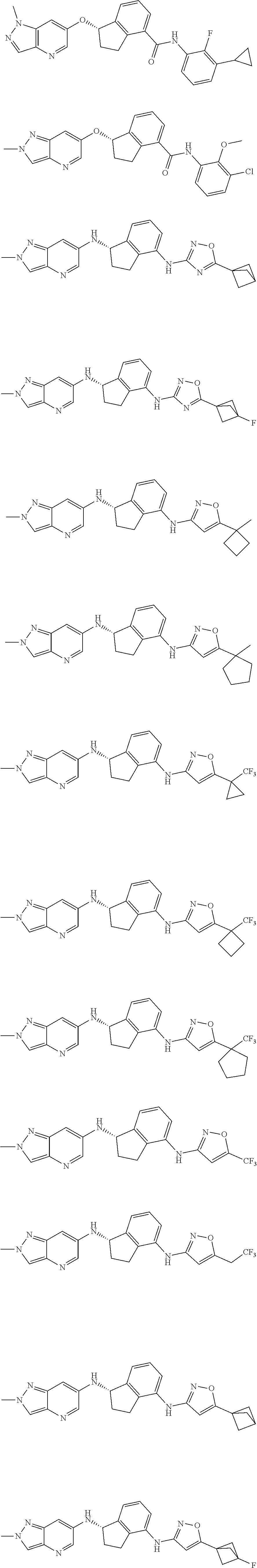

In another aspect, the present disclosure provides a compound having the structure of formula (V) or a pharmaceutically acceptable salt, stereoisomers, isotopic isomers thereof,

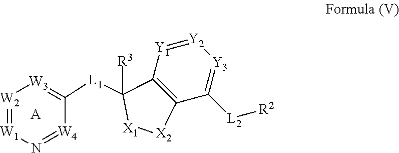

Wherein, X₁ represents O, S, NR^{a}, (CR^{L}R^{L′})ₘ, C(O);
Wherein, X₂ represents O, S, NR^{a}, (CR^{T}R^{T′})ₙ, C(O);
wherein, W₁ independently represents CR^{W1} or N;
wherein, W₂ independently represents CR^{W2} or N;
wherein, W₃ independently represents CR^{W3} or N;
wherein, W₄ independently represents CR^{W4} or N;
wherein, Y₁ independently represents CR^{Y1} or N;
wherein, Y₂ independently represents CR^{Y2} or N;
wherein, Y₃ independently represents CR^{Y3} or N;
wherein, each R^{W1}, R^{W2}, R^{W3}, R^{W4}, R^{Y1}, R^{Y2}, R^{Y3} independently represents hydrogen, deuterium, C₁-C₆ alkyl, deuterated C₁-C₆ alkyl, halogen, nitro, —OR^{a}, —SR^{a}, —P(O)R^{a}R^{b}, —CN, —S(O)₂R^{a}, —S(O)R^{a}, —SF₅, —NR^{a}R^{b}, —CR^{a}R^{b}NR^{a}R^{b}, —NR^{a}COR^{b}, —CONR^{a}R^{b}, C₁-C₆ haloalkyl, C₁-C₆ haloalkoxy, C₁-C₆ haloalkylthio, hydroxy (C₁-C₆alkyl); or each R^{W1}, R^{W2}, R^{W3}, R^{W4}, R^{Y1}, R^{Y2}, R^{Y3} independently represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
or W₁ and W₂ together with the atom which they are attached to form a 5-6 membered saturated or unsaturated ring fused to ring A, and the said ring may optionally contain 0, 1, 2 or 3 heteroatoms selected from O, N, or S;
wherein, L₁ represents absent, —(CR^{a}R^{b})ₒ—, —CR^{a}R^{b}O—, —OCR^{a}R^{b}—, —O—, —S—, —S(O)₂—, —C(O)—, —NR^{a}—, —S(O)₂NR^{a}—, —OC(O)—, —C(O)O—, —NR^{a}S(O)₂—, —CR^{a}R^{b}NR^{a}—, or —NR^{a}CR^{a}R^{b}—;
wherein, L₂ represents —C(O)NR^{a}— or —NR^{a}C(O)—;
wherein, R² represents a 5-10 membered heteroaryl, which is substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —S(O)₂R^{a}, —S(O)R^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, R³ represents hydrogen, halogen, C₁-C₆ haloalkyl, C₁-C₆ alkyl, or hydroxy;
or R³ and W₃ together with the atom which they are attached to form a 5-7 membered saturated or unsaturated ring fused to ring A, and the said ring may optionally contain 0, 1 or 2 heteroatoms selected from O, N, or S; and further, the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR⁶ or —CONR^{a}R^{b};
wherein, each R^{a} and R^{b} independently represents hydrogen, deuterium, halogen, C₁-C₆ alkyl, C₁-C₆ haloalkyl, hydroxy (C₁-C₆ alkyl), C₃-C₁₀ cycloalkyl, C₆-C₁₀ aryl, 5-10 membered heteroaryl; or R^{a} and R^{b} together with the carbon atom which they are attached to form a 3-6 membered ring, and the said ring may optionally contain 0, 1 or 2 heteroatoms selected from O, N, and S; and further, the said ring may optionally be substituted by halogen, hydroxy or C₁-C₆ alkyl;
wherein, each R^{L} and R^{L′} independently represents hydrogen, deuterium, halogen, C₁-C₆ alkyl, C₁-C₆ haloalkyl, hydroxy (C₁-C₆ alkyl), C₃-C₁₀ cycloalkyl; or R^{L} and R^{L′} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, each R^{T} and R^{T′} independently represents hydrogen, deuterium, halogen, C₁-C₆ alkyl, C₁-C₆ haloalkyl, hydroxy (C₁-C₆ alkyl), C₃-C₁₀ cycloalkyl; or R^{T} and R^{T′} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, o, m, n represents the integer of 1, 2 or 3.

In some embodiments of the present disclosure, wherein W₁ represents CH, CCH₃, CF, CCF₃, CCHF₂, CCF₂H, C(OCH₃), CNH₂, CNHCH₃, CN(CH₃)₂, CCH₂NHCH₃, CCH₂N(CH₃)₂, C(CN) or N.

In some embodiments of the present disclosure, wherein W₃ represents CH, CCH₃, CF, CCF₃, CCHF₂, CCH₂F, C(OCH₃), CNH₂, CNHCH₃, C(CN) or N

In some embodiments of the present disclosure, wherein W₄ represents CH, CCH₃, CF, CCF₃, CCHF₂, CCH₂F, C(OCH₃), CNH₂, CNHCH₃, C(CN) or N

In some embodiments of the present disclosure, wherein Y₁ represents CH, CCH₃, CF, CCl, CCF₃, CCHF₂, CCH₂F, C(OCH₃), C(CN) or N.

In some embodiments of the present disclosure, wherein Y₂ represents CH, CCH₃, CF, CCl, CCF₃, CCHF₂, CCH₂F, C(OCH₃), C(CN) or N.

In some embodiments of the present disclosure, wherein Y₃ represents CH, CCH₃, CF, CCl, CCF₃, CCHF₂, CCH₂F, C(OCH₃), C(CN) or N.

In some embodiments of the present disclosure, wherein X₁ represents —CH₂—, —CF₂—, —CHF—, —O—, —S—, —CHOH—, —CH₂CH₂— or —C(CH₃)₂.

In some embodiments of the present disclosure, wherein X₂ represents —CH₂—, —CF₂—, —CHF—, —O—, —S—, —CHOH—, —CH₂CH₂— or —C(CH₃)₂.

In some embodiments of the present disclosure, wherein L₁ represents —CH₂—, —CHF—, —CF₂—, —O—, —NH— or —NCH₃—.

In some embodiments of the present disclosure, wherein R^{W2} represents C₁-C₆ alkyl, halogen, —OH, O(C₁-C₆ alkyl); or R^{W2} represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), NR^{a}R^{b}, —CR^{a}R^{b}NR^{a}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein R^{W2} represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein R^{W2} represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, —OR^{a}, hydroxy (C₁-C₆ alkyl), —CN or C₁-C₆ haloalkyl.

In some embodiments of the present disclosure, wherein R^{W2} represents a 5-10 membered heteroaryl, which is substituted by 0-4 substituents selected from deuterium, halogen, C₁-C₆ alkyl, —OR^{a}, hydroxy (C₁-C₆ alkyl), —CN or C₁-C₆ haloalkyl.

In some embodiments of the present disclosure, wherein R^{W2} represents pyrazolyl, triazolyl, oxazolyl, isoxazolyl, thiazolyl, imidazolyl, which are substituted by 0-4 substituents selected from deuterium, halogen, C₁-C₆ alkyl, —OR^{a}, hydroxy (C₁-C₆ alkyl), —CN or C₁-C₆ haloalkyl.

In some embodiments of the present disclosure, wherein R^{W2} represents

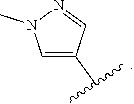 [SMILES: *c1cnn(C)c1]

In some embodiments of the present disclosure, wherein R² represents a 5-10 membered heteroaryl, which is substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein R² represents 5-10 membered heteroaryl, which is substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, or —C(O)R^{a}.

In some embodiments of the present disclosure, wherein W₁ and W₂ combine to form a ring fused to ring A having the following structure:

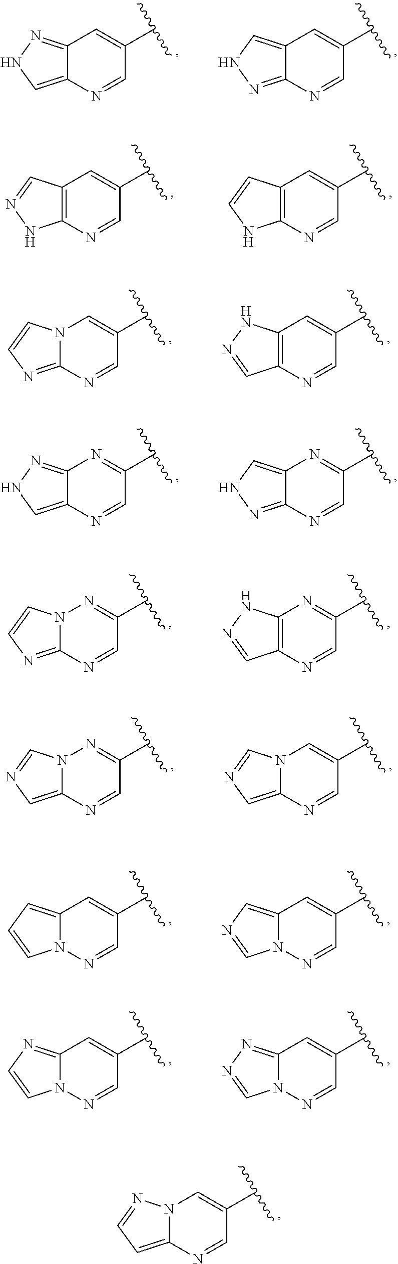

wherein the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}; and wherein the wavy line indicates the point of attachment with L₁.

In some embodiments of the present disclosure, wherein W₁ and W₂ combine to form a ring fused to ring A having the following structure:

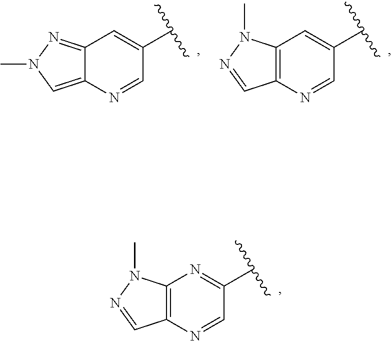 [SMILES: *c1cnc2cn(C)nc2c1 ; *c1cnc2cnn(C)c2c1 ; *c1cnc2cnn(C)c2n1]

wherein the wavy line indicates the point of attachment with L₁.

In some embodiments of the present disclosure, wherein R² represents pyridinyl, indazolyl, indolyl, thiazolyl, isothiazolyl, imidazolyl, pyrazolyl, oxazolyl, isoxazolyl, 1,2,4-oxadiazolyl, 1,3,4-oxadiazolyl, pyridazinyl, piperazinyl, pyrazinyl, benzothienyl, 1,2-benzoisoxazolyl, benzo[d]isothiazolyl, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R² represents

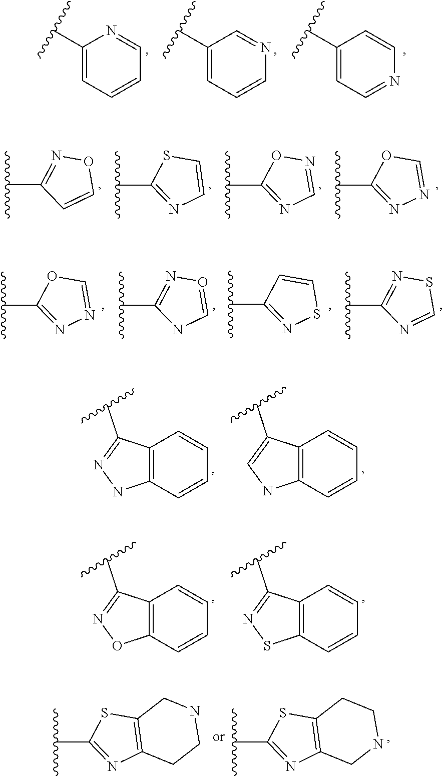

which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R³ represents hydrogen or CH₃.

The present disclosure provides a compound having the structure of formula (V) or a pharmaceutically acceptable salt, stereoisomers, isotopic isomers thereof,

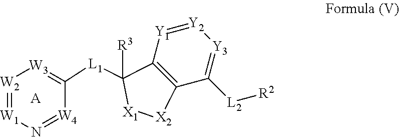

Wherein, X₁ represents O, S, NR^{a}, (CR^{L}R^{L′})ₘ, C(O);
Wherein, X₂ represents O, S, NR^{a}, (CR^{T}R^{T′})ₙ, C(O);
wherein, W₁ independently represents CR^{W1} or N;
wherein, W₂ independently represents CR^{W2};
wherein, W₃ independently represents CR^{W3} or N;
wherein, W₄ independently represents CR^{W4} or N;
wherein, Y₁ independently represents CR^{Y1} or N;
wherein, Y₂ independently represents CR^{Y2} or N;
wherein, Y₃ independently represents CR^{Y3} or N;
wherein, each R^{W1}, R^{W2}, R^{W3}, R^{W4}, R^{Y1}, R^{Y2}, R^{Y3} independently represents hydrogen, deuterium, C₁-C₆ alkyl, deuterated C₁-C₆ alkyl, halogen, nitro, —OR^{a}, —SR^{a}, —P(O)R^{a}R^{b}, —CN, —S(O)₂R^{a}, —S(O)R^{a}, —SF₅, —NR^{a}R^{b}, —CR^{a}R^{b}NR^{a}R^{b}, —NR^{a}COR^{b}, —CONR^{a}R^{b}, C₁-C₆ haloalkyl, C₁-C₆ haloalkoxy, C₁-C₆ haloalkylthio, hydroxy (C₁-C₆alkyl); or each R^{W1}, R^{W2}, R^{W3}, R^{W4}, R^{Y1}, R^{Y2}, R^{Y3} independently represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
or W₁ and W₂ together with the atom which they are attached to form a 5-6 membered saturated or unsaturated ring fused to ring A, and the said ring may optionally contain 0, 1, 2 or 3 heteroatoms selected from O, N, or S;
wherein, L₁ represents absent, —(CR^{a}R^{b})ₒ—, —CR^{a}R^{b}O—, —OCR^{a}R^{b}—, —O—, —S—, —S(O)₂—, —C(O)—, —NR^{a}—, —S(O)₂NR^{a}—, —OC(O)—, —C(O)O—, —NR^{a}S(O)₂—, —CR^{a}R^{b}NR^{a}—, or —NR^{a}CR^{a}R^{b}—;
wherein, L₂ represents —C(O)NR^{a}— or —NR^{a}C(O)—;
wherein, R² represents a 5-10 membered heteroaryl, which is substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —S(O)₂R^{a}, —S(O)R^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, R³ represents hydrogen, halogen, C₁-C₆ haloalkyl, C₁-C₆ alkyl, or hydroxy;
or R³ and W₃ together with the atom which they are attached to form a 5-7 membered saturated or unsaturated ring fused to ring A, and the said ring may optionally contain 0, 1 or 2 heteroatoms selected from O, N, or S; and further, the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, each R^{a} and R^{b} independently represents hydrogen, deuterium, halogen, C₁-C₆ alkyl, C₁-C₆ haloalkyl, hydroxy (C₁-C₆alkyl), C₃-C₁₀ cycloalkyl, C₆-C₁₀ aryl, 5-10 membered heteroaryl; or R^{a} and R^{b} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, each R^{L} and R^{L′} independently represents hydrogen, deuterium, halogen, C₁-C₆ alkyl, C₁-C₆ haloalkyl, hydroxy (C₁-C₆ alkyl), C₃-C₁₀ cycloalkyl; or R^{L} and R^{L′} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, each R^{T} and R^{T′} independently represents hydrogen, deuterium, halogen, C₁-C₆ alkyl, C₁-C₆ haloalkyl, hydroxy (C₁-C₆ alkyl), C₃-C₁₀ cycloalkyl; or R^{T} and R^{T′} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, o, m, n represents the integer of 1, 2 or 3.

In some embodiments of the present disclosure, wherein W₁ represents CH, CCH₃, CF, CCF₃, CCHF₂, CCF₂H, C(OCH₃), CNH₂, CNHCH₃, CN(CH₃)₂, CCH₂NHCH₃, CCH₂N(CH₃)₂, C(CN) or N.

In some embodiments of the present disclosure, wherein W₃ represents CH, CCH₃, CF, CCF₃, CCHF₂, CCH₂F, C(OCH₃), CNH₂, CNHCH₃, C(CN) or N.

In some embodiments of the present disclosure, wherein W₄ represents CH, CCH₃, CF, CCF₃, CCHF₂, CCH₂F, C(OCH₃), CNH₂, CNHCH₃, C(CN) or N.

In some embodiments of the present disclosure, wherein Y₁ represents CH, CCH₃, CF, CCl, CCF₃, CCHF₂, CCH₂F, C(OCH₃), C(CN) or N.

In some embodiments of the present disclosure, wherein Y₂ represents CH, CCH₃, CF, CCl, CCF₃, CCHF₂, CCH₂F, C(OCH₃), C(CN) or N.

In some embodiments of the present disclosure, wherein Y₃ represents CH, CCH₃, CF, CCl, CCF₃, CCHF₂, CCH₂F, C(OCH₃), C(CN) or N.

In some embodiments of the present disclosure, wherein X₁ represents —CH₂—, —CF₂—, —CHF—, —O—, —S—, —CHOH—, —CH₂CH₂— or —C(CH₃)₂.

In some embodiments of the present disclosure, wherein X₂ represents —CH₂—, —CF₂—, —CHF—, —O—, —S—, —CHOH—, —CH₂CH₂— or —C(CH₃)₂.

In some embodiments of the present disclosure, wherein L₁ represents —CH₂—, —CHF—, —CF₂—, —O—, —NH— or —NCH₃—.

In some embodiments of the present disclosure, wherein R^{W2} represents C₁-C₆ alkyl, halogen, —OH, O(C₁-C₆ alkyl); or R^{W2} represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein R^{W2} represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein R^{W2} represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, —OR^{a}, hydroxy (C₁-C₆ alkyl), —CN or C₁-C₆ haloalkyl.

In some embodiments of the present disclosure, wherein R^{W2} represents a 5-10 membered heteroaryl, which is substituted by 0-4 substituents selected from deuterium, halogen, C₁-C₆ alkyl, —OR³, hydroxy (C₁-C₆ alkyl), —CN or C₁-C₆ haloalkyl.

In some embodiments of the present disclosure, wherein R^{W2} represents pyrazolyl, triazolyl, oxazolyl, isoxazolyl, thiazolyl, imidazolyl, which are substituted by 0-4 substituents selected from deuterium, halogen, C₁-C₆ alkyl, —OR^{a}, hydroxy (C₁-C₆ alkyl), —CN or C₁-C₆ haloalkyl.

In some embodiments of the present disclosure, wherein R^{W2} represents

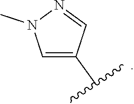 [SMILES: *c1cnn(C)c1]

In some embodiments of the present disclosure, wherein R² represents a 5-10 membered heteroaryl, which is substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}f, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein W₁ and W₂ combine to form a ring fused to ring A having the following structure:

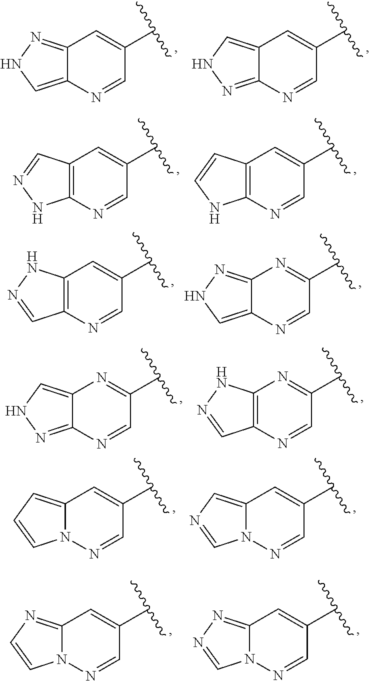

wherein the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}; and wherein the wavy line indicates the point of attachment with L₁.

In some embodiments of the present disclosure, wherein W₁ and W₂ combine to form a ring fused to ring A having the following structure:

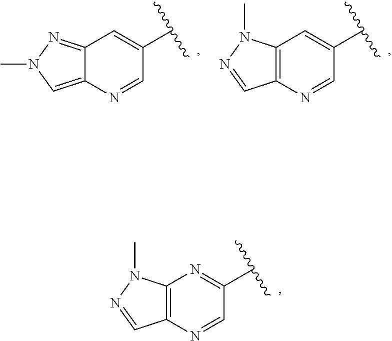 [SMILES: *c1cnc2cn(C)nc2c1 ; *c1cnc2cnn(C)c2c1 ; *c1cnc2cnn(C)c2n1]

wherein the wavy line indicates the point of attachment with L₁.

In some embodiments of the present disclosure, wherein R² represents pyridinyl, indazolyl, indolyl, thiazolyl, isothiazolyl, imidazolyl, pyrazolyl, oxazolyl, isoxazolyl, 1,2,4-oxadiazolyl, 1,3,4-oxadiazolyl, pyridazinyl, piperazinyl, pyrazinyl, benzothienyl, 1,2-benzoisoxazolyl, benzo[d]isothiazolyl, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R² represents

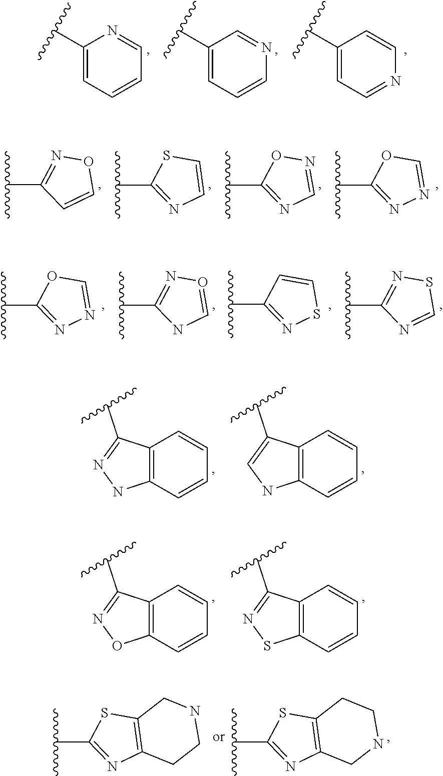

which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.
In some embodiments of the present disclosure, wherein R³ represents hydrogen or CH₃.

The present disclosure provides a compound having the structure of formula (V) or a pharmaceutically acceptable salt, stereoisomers, isotopic isomers thereof,

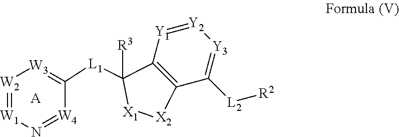

Wherein, X₁ represents O, S, NR^{a}, (CR^{L}R^{L′})ₘ, C(O);
Wherein, X₂ represents O, S, NR^{a}, (CR^{T}R^{T′})ₙ, C(O);
wherein, W₁ independently represents CR^{W1} or N;
wherein, W₂ independently represents N;
wherein, W₃ independently represents CR^{W3} or N;
wherein, W₄ independently represents CR^{W4} or N;
wherein, Y₁ independently represents CR^{Y1} or N;
wherein, Y₂ independently represents CR^{Y2} or N;
wherein, Y₃ independently represents CR^{Y3} or N;
wherein, each R^{W1}, R^{W3}, R^{W4}, R^{Y1}, R^{Y2}, R^{Y3} independently represents hydrogen, deuterium, C₁-C₆ alkyl, deuterated C₁-C₆ alkyl, halogen, nitro, —OR^{a}, —SR^{a}, —P(O)R^{a}R^{b}, —CN, —S(O)₂R^{a}, —S(O)R^{a}, —SF₅, —NR^{a}R^{b}, —CR^{a}R^{b}NR^{a}R^{b}, —NR^{a}COR^{b}, —CONR^{a}R^{b}, C₁-C₆ haloalkyl, C₁-C₆ haloalkoxy, C₁-C₆ haloalkylthio, hydroxy (C₁-C₆ alkyl); or each R^{W1}, R^{W2}, R^{W3}, R^{W4}, R^{Y1}, R^{Y2}, R^{Y3} independently represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
or W₁ and W₂ together with the atom which they are attached to form a 5-6 membered saturated or unsaturated ring fused to ring A, and the said ring may optionally contain 0, 1, 2 or 3 heteroatoms selected from O, N, or S;
wherein, L₁ represents absent, —(CR^{a}R^{b})ₒ—, —CR^{a}R^{b}O—, —OCR^{a}R^{b}—, —O—, —S—, —S(O)₂—, —C(O)—, —NR^{a}—, —S(O)₂NR^{a}—, —OC(O)—, —C(O)O—, —NR^{a}S(O)₂—, —CR^{a}R^{b}NR^{a}—, or —NR^{a}CR^{a}R^{b}—;
wherein, L₂ represents —C(O)NR^{a}— or —NR^{a}C(O)—;
wherein, R² represents a 5-10 membered heteroaryl, which is substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —S(O)₂R^{a}, —S(O)R^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, R³ represents hydrogen, halogen, C₁-C₆ haloalkyl, C₁-C₆ alkyl, or hydroxy;
or R³ and W₃ together with the atom which they are attached to form a 5-7 membered saturated or unsaturated ring fused to ring A, and the said ring may optionally contain 0, 1 or 2 heteroatoms selected from O, N, or S; and further, the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, each R^{a} and R^{b} independently represents hydrogen, deuterium, halogen, C₁-C₆ alkyl, C₁-C₆ haloalkyl, hydroxy (C₁-C₆ alkyl), C₃-C₁₀ cycloalkyl, C₆-C₁₀ aryl, 5-10 membered heteroaryl; or R^{a} and R^{b} together with the carbon atom which they are attached to form a 3-6 membered ring, and the said ring may optionally contain 0, 1 or 2 heteroatoms selected from O, N, and S; and further, the said ring may optionally be substituted by halogen, hydroxy or C₁-C₆ alkyl;
wherein, each R^{L} and R^{L′} independently represents hydrogen, deuterium, halogen, C₁-C₆ alkyl, C₁-C₆ haloalkyl, hydroxy (C₁-C₆ alkyl), C₃-C₁₀ cycloalkyl; or R^{L} and R^{L′} together with the carbon atom which they are attached to form a 3-6 membered ring, and the said ring may optionally contain 0, 1 or 2 heteroatoms selected from O, N, or S; and further, the said ring may optionally be substituted by halogen, hydroxy (C₁-C₆ alkyl);
wherein, each R^{T} and R^{T′} independently represents hydrogen, deuterium, halogen, C₁-C₆ alkyl, C₁-C₆ haloalkyl, hydroxy (C₁-C₆ alkyl), C₃-C₁₀ cycloalkyl; or R^{T} and R^{T′} together with the carbon atom which they are attached to form a 3-6 membered ring, and the said ring may optionally contain 0, 1 or 2 heteroatoms selected from O, N, or S; and further, the said ring may optionally be substituted by halogen, hydroxy (C₁-C₆ alkyl);
wherein, o, m, n represents the integer of 1, 2 or 3.

In some embodiments of the present disclosure, wherein W₁ represents CH, CCH₃, CF, CCF₃, CCHF₂, CCF₂H, C(OCH₃), CNH₂, CNHCH₃, CN(CH₃)₂, CCH₂NHCH₃, CCH₂N(CH₃)₂, C(CN) or N.

In some embodiments of the present disclosure, wherein W₃ represents CH, CCH₃, CF, CCF₃, CCHF₂, CCH₂F, C(OCH₃), CNH₂, CNHCH₃, C(CN) or N.

In some embodiments of the present disclosure, wherein W₄ represents CH, CCH₃, CF, CCF₃, CCHF₂, CCH₂F, C(OCH₃), CNH₂, CNHCH₃, C(CN) or N.

In some embodiments of the present disclosure, wherein Y₁ represents CH, CCH₃, CF, CCl, CCF₃, CCHF₂, CCH₂F, C(OCH₃), C(CN) or N.

In some embodiments of the present disclosure, wherein Y₂ represents CH, CCH₃, CF, CCl, CCF₃, CCHF₂, CCH₂F, C(OCH₃), C(CN) or N.

In some embodiments of the present disclosure, wherein Y₃ represents CH, CCH₃, CF, CCl, CCF₃, CCHF₂, CCH₂F, C(OCH₃), C(CN) or N.

In some embodiments of the present disclosure, wherein X₁ represents —CH₂—, —CF₂—, —CHF—, —O—, —S—, —CHOH—, —CH₂CH₂— or —C(CH₃)₂.

In some embodiments of the present disclosure, wherein X₂ represents —CH₂—, —CF₂—, —CHF—, —O—, —S—, —CHOH—, —CH₂CH₂— or —C(CH₃)₂.

In some embodiments of the present disclosure, wherein L₁ represents —CH₂—, —CHF—, —CF₂—, —O—, —NH— or —NCH₃—.

In some embodiments of the present disclosure, wherein R² represents a 5-10 membered heteroaryl, which is substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} for —CONR^{a}R^{b}.

In some embodiments of the present disclosure, wherein W₁ and W₂ combine to form a ring fused to ring A having the following structure:

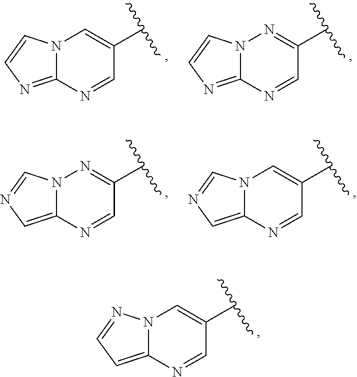 [SMILES: *c1cnc2nccn2c1 ; *c1cnc2nccn2n1 ; *c1cnc2cncn2n1 ; *c1cnc2cncn2c1 ; *c1cnc2ccnn2c1]

wherein the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}; and wherein the wavy line indicates the point of attachment with L₁.

In some embodiments of the present disclosure, wherein R² represents pyridinyl, indazolyl, indolyl, thiazolyl, isothiazolyl, imidazolyl, pyrazolyl, oxazolyl, isoxazolyl, 1,2,4-oxadiazolyl, 1,3,4-oxadiazolyl, pyridazinyl, piperazinyl, pyrazinyl, benzothienyl, 1,2-benzoisoxazolyl, benzo[d]isothiazolyl, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R² represents

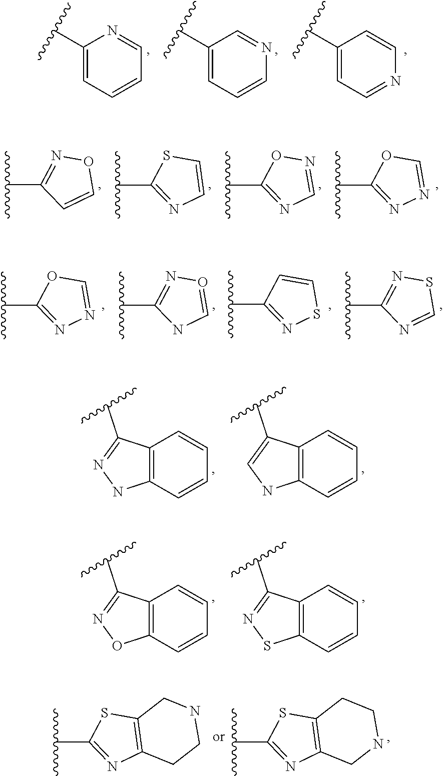

which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R³ represents hydrogen or CH₃.

The present disclosure provides a compound having the structure of formula (VI) or a pharmaceutically acceptable salt, stereoisomers, isotopic isomers thereof,

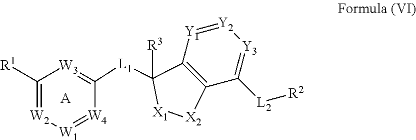

Wherein, X₁ represents O, S, NR^{a}, (CR^{L}R^{L′})ₘ, C(O);
Wherein, X₂ represents O, S, NR^{a}, (CR^{T}R^{T′})ⁿ, C(O);
wherein, W₂ independently represents CR^{W1} or N;
wherein, W₂ independently represents CR^{W2} or N;
wherein, W₃ independently represents CR^{W3} or N;
wherein, W₄ independently represents CR^{W4} or N;
wherein, Y₂ independently represents CR^{Y1} or N;
wherein, Y₂ independently represents CR^{Y3} or N;
wherein, Y₃ independently represents CR^{Y3} or N;
wherein, each R^{W1}, R^{W2}, R^{W3}, R^{W4}, R^{Y1}, R^{Y2}, R^{Y3} independently represents hydrogen, deuterium, C₁-C₆ alkyl, deuterated C₁-C₆ alkyl, halogen, nitro, —OR^{a}, —SR^{a}, —P(O)R^{a}R^{b}, —CN, —S(O)₂R^{a}, —S(O)R^{a}, —SF₅, —NR^{a}R^{b}, —CR^{a}R^{b}NR^{a}R^{b}, —NR^{a}COR^{b}, —CONR^{a}R^{b}, C₁-C₆ haloalkyl, C₁-C₆ haloalkoxy, C₁-C₆ haloalkylthio, hydroxy (C₁-C₆alkyl); or each R^{W1}, R^{W2}, R^{W3}, R^{W4}, R^{Y1}, R^{Y2}, R^{Y3} independently represents C₃-C₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
or W₁ and W₂ together with the atom which they are attached to form a 5-6 membered saturated or unsaturated ring fused to ring A, and the said ring may optionally contain 0, 1, 2 or 3 heteroatoms selected from O, N, or S;
or W₁ and W₄ together with the atom which they are attached to form a 5-6 membered saturated or unsaturated ring fused to ring A, and the said ring may optionally contain 0, 1, 2 or 3 heteroatoms selected from O, N, or S;
wherein, L₁ represents absent, —(CR^{a}R^{b})ₒ—, —CR^{a}R^{b}O—, —OCR^{a}R^{b}—, —O—, —S—, —S(O)₂—, —C(O)—, —NR^{a}—, —S(O)₂NR^{a}—, —OC(O)—, —C(O)O—, —NR^{a}S(O)₂—, —CR^{a}R^{b}NR^{a}—, or —NR^{a}CR^{a}R^{b}—;
wherein, L₂ represents absent, —(CR^{a}R^{b})ₒ—, —CR^{a}R^{b}O—, —OCR^{a}R^{b}—, —O—, —C(O)—, —NR^{a}—, —S(O)₂NR^{a}—, —OC(O)—, —C(O)O—, —NR³S(O)₂—, —CR^{a}R^{b}NR^{a}—, —NR^{a}CR^{a}R^{b}—, —NR^{a}C(O)NR^{b}—, —NR^{a}C(O)NR^{b}CR^{a}R^{b}—, —NR^{a}C(O)O—, —NR^{a}C(O)—, or —C(O)NR^{a}—.

Wherein, R¹ represents hydrogen, deuterium, C₁-C₆ alkyl, deuterated C₁-C₆ alkyl, halogen, —OR^{a}, —SR^{a}, —P(O)R^{a}R^{b}, —CN, —S(O)₂R^{a}, —S(O)R^{a}, —SF₅, —NR^{a}R^{b}, C₁-C₆ haloalkyl, C₁-C₆ haloalkoxy, C₁-C₆ haloalkylthio, hydroxy (C₁-C₆ alkyl); or R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, and 5-10 membered heteroaryl, which are substituted by 0-4 substituents that selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
or R¹ and W₂ together with the atom which they are attached to form a 5-6 membered saturated or unsaturated ring fused to ring A, and the said ring may optionally contain 0, 1 or 2 heteroatoms selected from O, N, or S; and further, the said ring may optionally substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, R² represents C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl; which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —S(O)₂R^{a}, —S(O)R^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, R³ represents hydrogen, halogen, C₁-C₆ haloalkyl, C₁-C₆ alkyl, or hydroxy;
or R³ and W₃ together with the atom which they are attached to form a 5-7 membered saturated or unsaturated ring fused to ring A, and the said ring may optionally contain 0, 1 or 2 heteroatoms selected from O, N, or S; and further, the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, each R^{a} and R^{b} independently represents hydrogen, C₁-C₆ alkyl, C₁-C₆ haloalkyl, hydroxy (C₁-C₆ alkyl), C₃-C₁₀ cycloalkyl; or R^{a} and R^{b} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, each R^{L} and R^{L′} independently represents hydrogen, C₁-C₆ alkyl, C₁-C₆ haloalkyl, hydroxy (C₁-C₆ alkyl), C₃-C₁₀ cycloalkyl; or R^{L} and R^{L′} together with the carbon atom which they are attached to form a 3-6 membered ring, and the said ring may optionally contain 0, 1 or 2 heteroatoms selected from O, N, or S; and further, the said ring may be optionally substituted by halogen, hydroxy or C₁-C₆ alkyl;
wherein, each R^{T} and R^{T′} independently represents hydrogen, C₁-C₆ alkyl, C₁-C₆ haloalkyl, hydroxy (C₁-C₆ alkyl), C₃-C₁₀ cycloalkyl; or R^{T} and R^{T′} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, o, m, n represents the integer of 1, 2 or 3.

In some embodiments of the present disclosure, wherein W₁ represents CH, CCH₃, CF, CCF₃, CCHF₂, CHF₂, COCH₃, C(CN) or N.

In some embodiments of the present disclosure, wherein W₁ represents N.

In some embodiments of the present disclosure, wherein W₂ represents CH, CCH₃, CF, CCF₃, CCHF₂, CHF₂, COCH₃, CNH₂, CNHCH₃, CN(CH₃)₂, CH₂NCH₃, CH₂N(CH₃)₂, C(CN) or N.

In some embodiments of the present disclosure, wherein W₃ represents CH, CCH₃, CF, CCF₃, CCHF₂, CHF₂, COCH₃, C(CN) or N.

In some embodiments of the present disclosure, wherein W₄ represents CH, CCH₃, CF, CCF₃, CCHF₂, CHF₂, COCH₃, CNH₂, CNHCH₃, C(CN) or N.

In some embodiments of the present disclosure, wherein Y₁ represents CH, CCH₃, CF, CCl, CCF₃, CCHF₂, CHF₂, COCH₃, C(CN) or N.

In some embodiments of the present disclosure, wherein Y₂ represents CH, CCH₃, CF, CCl, CCF₃, CCHF₂, CHF₂, COCH₃, C(CN) or N.

In some embodiments of the present disclosure, wherein Y₃ represents CH, CCH₃, CF, CCl, CCF₃, CCHF₂, CHF₂, COCH₃, C(CN) or N.

In some embodiments of the present disclosure, wherein X₁ represents CH₂, CF₂, CHF, O, S, CHOH, CH₂CH₂ or C(CH₃)₂.

In some embodiments of the present disclosure, wherein X₂ represents CH₂, CF₂, CHF, O, S, CHOH, CH₂CH₂ or C(CH₃)₂.

In some embodiments of the present disclosure, wherein L₁ represents CH₂, CF₂, O, NH or NCH₃.

In some embodiments of the present disclosure, wherein L₂ represents —NHC(O)—, —C(O)NH—, —NHC(O)NH—, —NHC(O)O—, —CH₂NH—, —NHCH₂—, —CH₂O—, —OCH₂— or —NH—.

In some embodiments of the present disclosure, wherein R¹ represents C₁-C₆ alkyl, halogen, —OH, O(C₁-C₆ alkyl); or R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, —OR^{a}, hydroxy (C₁-C₆ alkyl), —CN or C₁-C₆ haloalkyl.

In some embodiments of the present disclosure, wherein R¹ represents a 5-10 membered heteroaryl, which is substituted by 0-4 substituents selected from deuterium, halogen, C₁-C₆ alkyl, —OR^{a}, hydroxy (C₁-C₆ alkyl), —CN or C₁-C₆ haloalkyl.

In some embodiments of the present disclosure, wherein R¹ represents pyrazolyl, triazolyl, oxazolyl, thiazolyl, imidazolyl, which are substituted by 0-4 substituents selected from deuterium, halogen, C₁-C₆ alkyl, —OR^{a}, hydroxy (C₁-C₆ alkyl), —CN or C₁-C₆ haloalkyl.

In some embodiments of the present disclosure, wherein R¹ represents

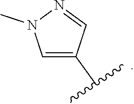 [SMILES: *c1cnn(C)c1]

In some embodiments of the present disclosure, wherein R² represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryls, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

In some embodiments of the present disclosure, wherein R¹ and W₂ combine to form a ring fused to ring A having the following structure:

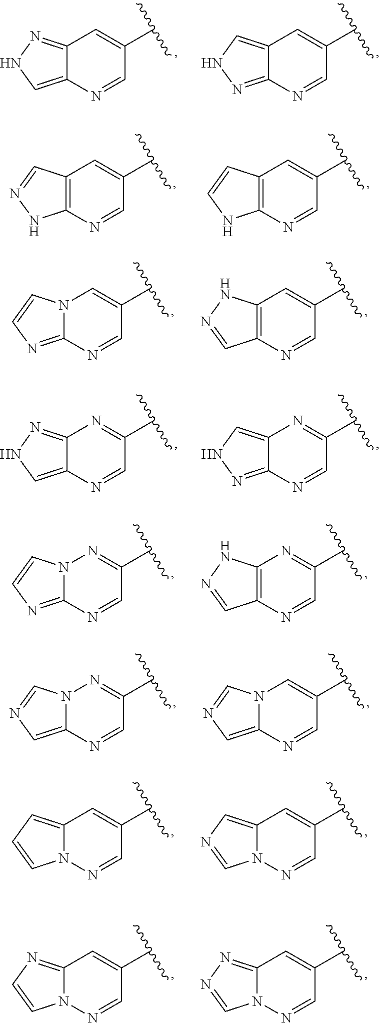

wherein the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}; and wherein the wavy line indicates the point of attachment with L₁.

In some embodiments of the present disclosure, wherein R¹ and W₂ combine to form a ring fused to ring A having the following structure:

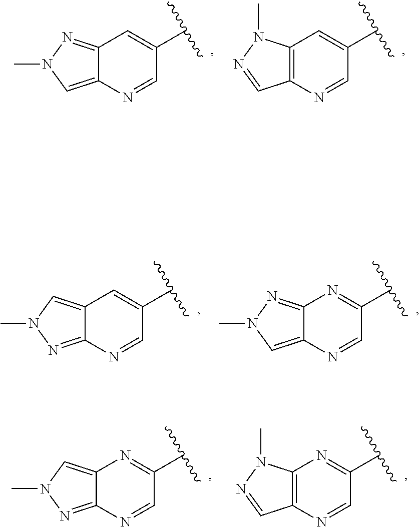 [SMILES: *c1cnc2cn(C)nc2c1 ; *c1cnc2cnn(C)c2c1 ; *c1cnc2nn(C)cc2c1 ; *c1cnc2cn(C)nc2n1 ; *c1cnc2nn(C)cc2n1 ; *c1cnc2cnn(C)c2n1]

wherein the wavy line indicates the point of attachment with L₁.

In some embodiments of the present disclosure, wherein R² represents phenyl, pyridinyl, indazolyl, indolyl, thiazolyl, isothiazolyl, imidazolyl, pyrazolyl, oxazolyl, isoxazolyl, 1,2,4-oxadiazolyl, 1,3,4-oxadiazolyl, pyridazinyl, piperazinyl, pyrazinyl, cyclohexyl, benzothienyl, 1,2-benzoisoxazolyl, benzo[d] isothiazolyl, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R² represents:

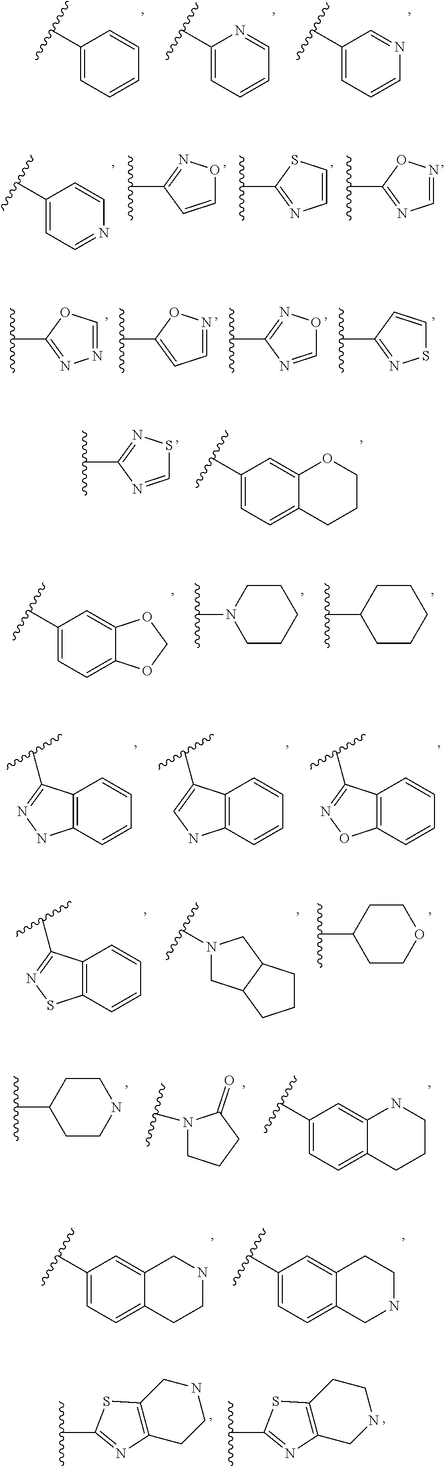

which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R² represents:

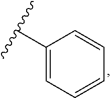 [SMILES: *c1ccccc1]

which is substituted by 0-4 substituents selected from deuterium, halogen, C₁-C₆ alkyl, C₂-C₆ alkenyl, C₂-C₆alkynvl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, C₁-C₆ haloalkyl, or C₁-C₆ alkoxy.

In some embodiments of the present disclosure, wherein R³ represents hydrogen or CH₃.

Specifically, the present disclosure provides compounds having the following structures:

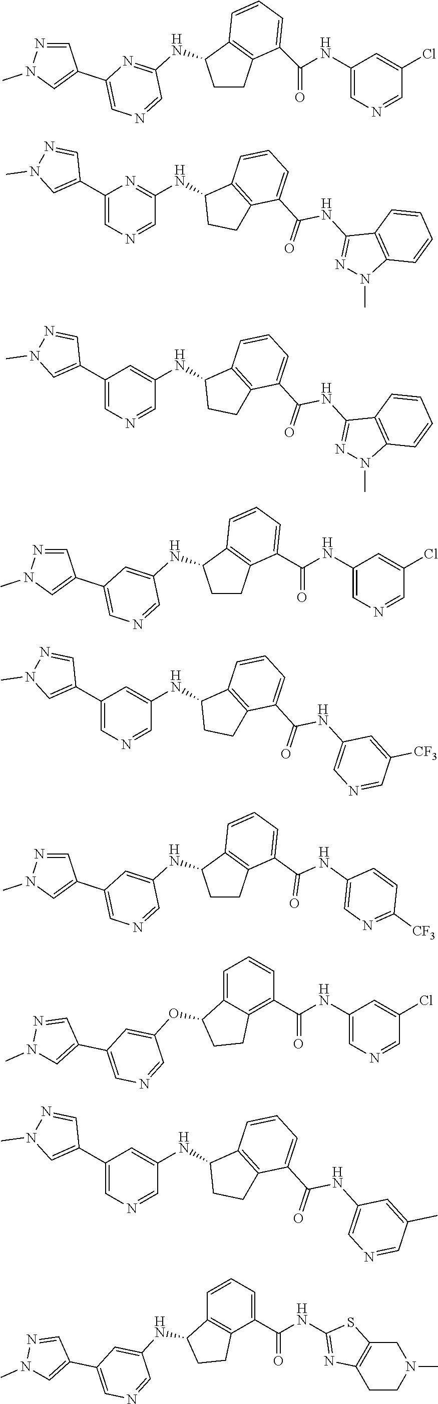

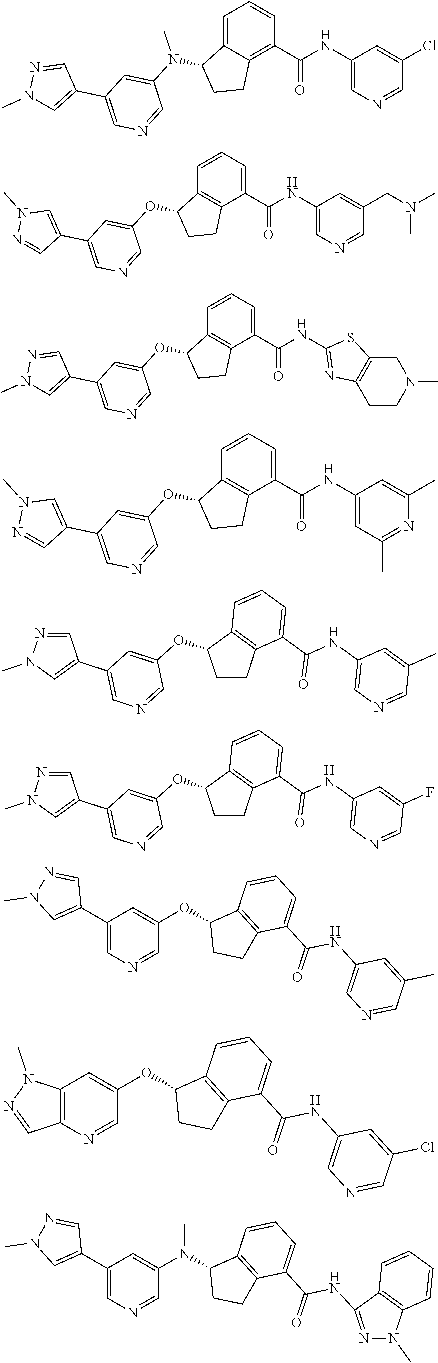

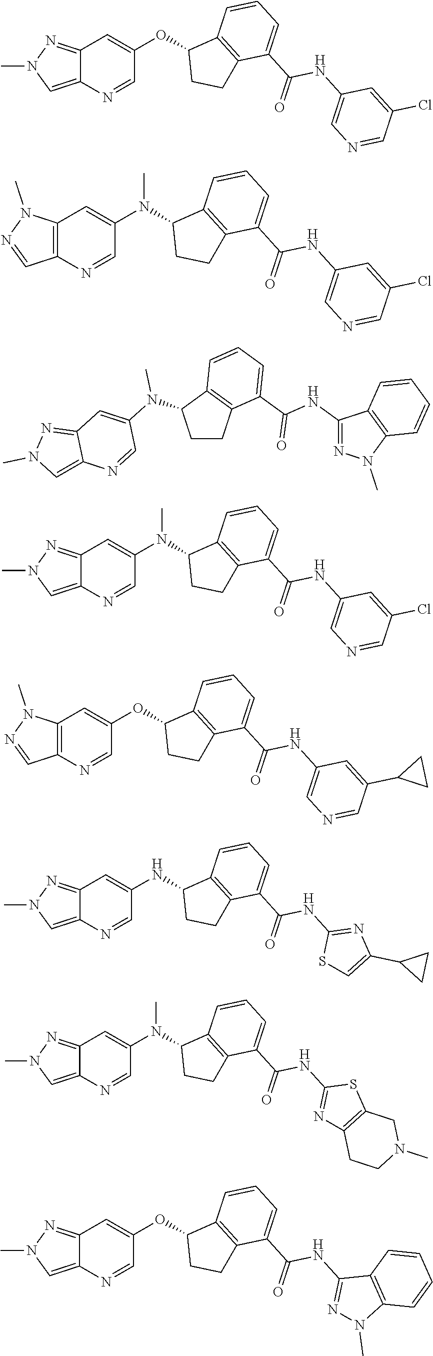

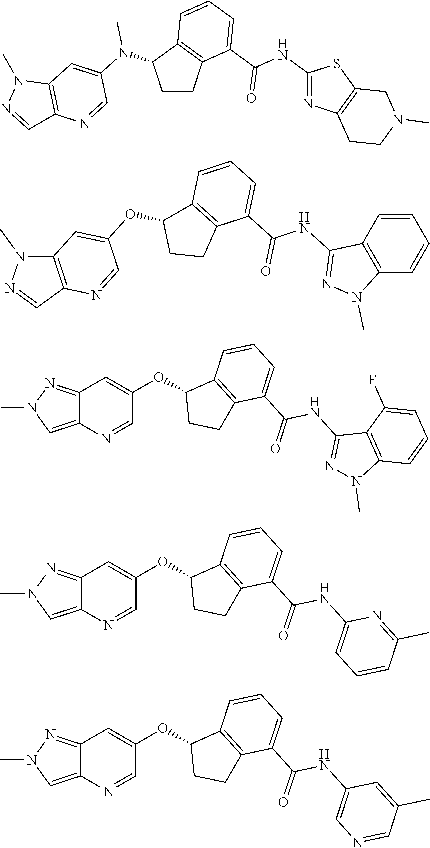

In addition, the present disclosure further provides a pharmaceutical composition, wherein, the pharmaceutical composition comprises the aforementioned compound, its tautomer, stereoisomer, hydrate, solvate, pharmaceutically acceptable salt or prodrug thereof, and a pharmaceutically acceptable excipients.

It should be understood that within the scope of the present invention, the above technical features of the present invention and the technical features specifically described in the following (e.g., embodiments) can be combined with each other, thereby forming a new or preferred technical solution. Due to space limitations, it will not be repeated herein.

### Definitions

Unless otherwise stated, the definitions of groups and terms specified in the specification and claims of the present application, including definitions of examples, exemplary definitions, preferred definitions, definitions recorded in the table, definitions of specific compounds in the embodiments, and the like, can be arbitrarily combined and combined with each other. Such a combination and a combined group definition and compound structure should fall within the scope of the description of the present application.

Unless otherwise defined, the technical terms used herein have the same meaning as commonly understood by a person skilled in the art to which the subject matter belongs. Unless otherwise stated, all patents, patent applications, and publications cited herein are incorporated herein by reference in their entirety. If there are a plurality of definitions to the terms herein, the definition of the present chapter is defined.

It is to be understood that both the foregoing general description and the following detailed description are exemplary and are for explanation only and are not intended to limit the subject matter of the present invention. In the present application, unless otherwise specifically stated, the singular also includes the plural. It must be noted that the singular forms used in this specification and the claims include the plural forms of the recited thing, unless expressly stated otherwise herein.

It should also be noted that, unless otherwise stated, “or”, “or” means “and/or”. In addition, the terms “comprising” and other forms, such as “comprising”, “containing”, and “containing” are not intended to be limiting.

Unless otherwise specified, conventional methods within the art, such as mass spectrometry, NMR, IR and UV/VIS spectroscopy, and pharmacological methods, are employed. Unless specifically defined, the terms used herein to analyze chemical, organic synthetic chemistry, and related descriptions of drugs and pharmaceutical chemistry are known in the art. Standard techniques may be used in chemical synthesis, chemical analysis, drug preparation, formulation and delivery, and in the treatment of patients. For example, the reaction and purification may be carried out using a manufacturer's description of the kit, or in a manner well known in the art or a description of the invention. The techniques and methods described above may generally be implemented in accordance with conventional methods well known in the art, in accordance with a number of summaries cited and discussed in this specification and in more specific documents. In the present specification, groups and substituents thereof may be selected by those skilled in the art to provide stable structural moieties and compounds.

Unless otherwise indicated, in addition to the specific structure of the compound, the compound of the present disclosure may also be extended and interpreted to further include a pharmaceutically acceptable salt, a stereoisomer, an isotopic isomer (for example, a deuterated compound), a solvate, a hydrate, a prodrug, and a metabolite of the compound, that is, a pharmaceutically acceptable salt, a stereoisomer, an isotopic isomer, a solvate, a hydrate, a prodrug, and a metabolite of the compound also fall within the scope of protection of the compound.

Preferably, the above pharmaceutical composition of the present disclosure may further comprise a second active substance, the second active substance is an anti-tumor drug, and the anti-tumor drug comprises one or more of a chemotherapeutic drug, a targeted tumor therapeutic drug, or a tumor therapeutic antibody drug.

In addition, the present disclosure also provides a compound of the present disclosure, a pharmaceutically acceptable salt, an ester, a prodrug, a stereoisomer or an isotope derivative thereof, a method for treating a disease by inhibiting CSFIR, VEGFR, PDGFR or DDR1, or combination thereof, and a preferred disease being a tumor.

Unless otherwise stated, the term “alkyl” itself or as part of another substituent refers to a straight-chain (ie, unbranched) or branched, or cyclic hydrocarbon group, or a combination thereof, which may be saturated, mono- or polyunsaturated, may include a divalent or polyvalent group having a specified number of carbon atoms (ie, C₁-C₁₀ refers to 1 to 10 carbon atoms). Examples of saturated hydrocarbyl groups include, but are not limited to, groups such as methyl, ethyl, n-propyl, isopropyl, n-butyl, t-butyl, isobutyl, sec-butyl, cyclohexyl, cyclohexylmethyl, cyclopropylmethyl and the like, such as n-pentyl, n-hexyl, n-heptyl, n-octyl and other homologues and isomers. An unsaturated alkyl group is an alkyl group having one or more double bonds or triple bonds. Examples of unsaturated alkyl groups include, but are not limited to, vinyl, 2-propenyl, crotyl, 2-isopentenyl, 2-(butadienyl), 2,4-pentadiene, 3-(1,4-pentadiene), ethynyl, 1-propynyl, 3-propynyl, 3-butynyl, and higher homologues and isomers. An alkyl group defined as a hydrocarbyl group is referred to as “homoalkyl”. The alkyl is optionally substituted with one or more halogen atoms.

The term “haloalkyl” or “halogenated alkyl” refers to an alkyl group as defined above, wherein one or more hydrogen atoms are replaced by halogen atoms.

The term “alkylene” itself or as part of another substituent refers to a divalent group derived from an alkyl group, such as, but not limited to, —CH₂CH₂CH₂CH₂—, —CH₂CH═CHCH₂—, —CH₂C≡CCH₂—, —CH₂CH₂CH(CH₂CH₂CH₃)CH₂—. Alkyl (or alkylene) typically has from 1 to 24 carbon atoms and the present disclosure preferably has a group having 10 or less carbon atoms. “Lower alkyl” or “lower alkylene” refers to a shorter alkyl or alkylene group, typically having 8 or fewer carbon atoms. The alkylene is optionally substituted with one or more halogen atoms.

The term “alkenyl” refers to an unsaturated branched or straight chain hydrocarbon group having at least one carbon-carbon double bond derived from the removal of a hydrogen atom from a single carbon atom of an olefin molecule. The group may be in the Z-form or E-form (cis or trans) surrounding one or more double bonds. Typical alkenyl groups include, but are not limited to, vinyl groups; propenyl (eg, prop-1-en-1-yl, prop-1-en-2-yl, prop-2-en-1-yl (allyl) and prop-2-en-2-yl); butenyl (eg, butyl-1-en-1-yl, butan-1-en-2-yl, 2-methyl-prop-1-en-1-yl, butan-2-en-1-yl, butan-2-en-1-yl, butan-2-en-2-yl, butyl-1,3-diene-1-yl and butyl-1,3-diene-2-yl); and the like. In some embodiments, the alkenyl group has from 2 to 20 carbon atoms, and in other embodiments, from 2 to 6 carbon atoms. Alkenyl groups having 2 to 6 carbon atoms may be referred to as (C₂-C₆) alkenyl groups.

The term “alkynyl” refers to a carbon chain containing at least one carbon-carbon triple bond, which may be linear or branched, or a combination thereof. Examples of alkynyl groups include ethynyl, propargyl,3-methyl-1-pentynyl, 2-heptynyl, and the like. The alkynyl group is optionally substituted with one or more halogen atoms.

The term “cycloalkyl” refers to a monocyclic or bicyclic saturated carbocyclic ring, each having 3 to 10 carbon atoms. “Condensed analogs” of cycloalkyl means that the monocyclic ring is fused to an aryl or heteroaryl group, wherein the attachment site is in the non-aromatic moiety. Examples of cycloalkyl and their Condensed analogs include cyclopropyl, cyclobutyl, cyclopentyl, cyclohexyl, tetrahydronaphthyl, decahydronaphthyl, dihydroindenyl, and the like. The cycloalkyl is optionally substituted with one or more halogen atoms. Further, the term “cycloalkyl” in the present disclosure includes a bridge ring system and a spiro ring system.

The term “cycloalkenyl” refers to a cyclic hydrocarbon group having a carbon-carbon double bond in a ring, the number of carbon-carbon double bonds is not limited on the basis of a valence bond principle, for example, 1, 2, or 3, and the definition of the cycloalkenyl group also includes a fused cycloalkenyl group formed by fused with a cycloalkyl group or other cycloalkenyl groups. Examples of cycloalkenyl groups include, but are not limited to, cyclopropene, cyclobutene, cyclopentene and cyclohexene, cyclobutadiene, cyclopentadiene, 1,3,5-cycloheptatriene, 1,3,5,7-cyclooctadiene, and the like.

The term “heterocyclic alkenyl” refers to a heterocyclic hydrocarbon group having a carbon-carbon double bond in the said ring, wherein the atoms comprising the said ring include, in addition to carbon atoms, at least one heteroatom selected from nitrogen, oxygen or sulfur. The heterocyclic alkenyl group may be non-aromatic.

The term “oxo” refers to the group formed by the substitution of two hydrogens on a carbon atom with the same oxygen, also known as carbonyl (═O). The N-oxide of an aromatic heterocycle may also be represented as an oxo-aromatic heterocycle, such as a pyridine N-oxide.

The term “alkylthio” refers to —SR, where R represents any straight or branched alkyl group. Examples of alkylthio include, but are not limited —SCH₃, —SCH₂CH₃, etc.

The term “hydroxy (C₁-C₆ alkyl)” or “hydroxy C₁-C₆ alkyl” refers to a group obtained by the substitution of hydrogen atoms of any of the C₁-C₆ alkyl groups with hydroxyl groups. The term “halo (C₁-C₆ alkyl)” refers to a group obtained by substitution of any hydrogen atom of C₁-C₆ alkyl with halo. The term “(CH₃)₂N—(C₁-C₆ alkyl)” means that any hydrogen atom of a C₁-C₆ alkyl group is replaced with a dimethylamino group.

The term “alkoxy” refers to a straight-chain or branched alkoxy group having a number of carbon atoms shown. C₁₋₆ alkoxy (C₁-C₆ alkoxy), for example, including methoxy, ethoxy, propoxy, isopropoxy, and the like.

Unless otherwise stated, the term “heteroalkyl” per se or in combination with another term refers to a stable straight-chain or branched, or cyclic hydrocarbyl group consisting of at least one carbon atom and at least one heteroatom selected from O, N, P, Si, S, or a cyclic hydrocarbon group, or a combination thereof, wherein the nitrogen atom, the phosphorus atom, or the sulfur atom may, optionally be oxidized and the nitrogen atom may optionally be quaternized. Heteroatoms O, N, P, S, and Si may be placed anywhere within the heteroalkyl group or at a location where the alkyl group is connected to the rest of the molecule. For example, but not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃ and —CN. Up to two or three heteroatoms may be continuous. For example, —CH₂—NH—OCH₃ and —CH₂—O—Si(CH₃)₃. Similarly, the term “heteroalkylene” alone or in combination with other terms means a divalent group derived from a heteroalkyl, such as, but not limited to, —CH₂—CH₂—S—CH₂—CH₂— and —CH₂—S—CH₂—CH₂—NH—CH₂—. For heteroalkylene, heteroatoms may be at either end or both ends of the chain (eg, alkyleneoxy, alkylene dioxy, alkylamine, alkylene diamino, etc.). Furthermore, for alkylene and heteroalkylene linking groups, the writing direction of molecular formula of the linking group does not represent the orientation of the linking group. For example, the formula —C(O)OR′— represents —C(O)OR′— and —R′OC(O)—. As noted above, heteroalkyl as used herein includes those groups attached to the rest of the molecule through heteroatoms, such as —C(O)R^{a}, —C(O)NR^{a}, —NR′R″, —OR^{a}, —SR′ and/or —SO₂R′. In referring to “heteroalkyl” and then to specific heteroalkyl groups such as —NR′R″, it should be understood that the terms heteroalkyl and —NR′R″ are not repetitive and mutually exclusive. Rather, these specific heteroalkyl groups are cited for clarity. Thus, the term “heteroalkyl” should not be construed as excluding specific heteroalkyl groups such as —NR′R″.

The term “cycloalkoxy” refers to a cycloalkyl group as defined above, such as cyclopropoxy, bonded to an oxygen atom.

The term “haloalkoxy” or “halogenated alkoxy” refers to an alkoxy group as defined above in which one or more hydrogen atoms are replaced by halo atoms.

The term “aryl” refers to a monocyclic or bicyclic aryl group containing only carbon atoms. The “Condensed analogs” of the aryl group refers to the fusing of an aryl group to a monocyclic cycloalkyl group or a monocyclic heterocyclic group, where the attachment point is located in the aryl moiety. Examples of aryl and fused ring analogs thereof include phenyl, naphthyl, indanyl, indenyl, tetrahydronaphthyl, 2,3-dihydrobenzofuran, dihydrobenzopyran, 1,4-benzodioxane, and the like.

The term “heteroaryl” refers to a monocyclic or bicyclic aryl group containing at least one heteroatom selected from N, O and S. The “Condensed analogs” of heteroaryl refers to the fusing of a heteroaryl group to a monocyclic cycloalkyl group or a monocyclic heterocyclic group, where the attachment point is located in the aryl moiety. Examples of heteroaryl groups include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furyl, triazinyl, thienyl, pyrimidinyl, pyridazinyl, pyrazinyl, benzoxazolyl, benzothiazolyl, benzimidazolyl, benzofuranyl, benzothienyl, furo(2,3-b)pyridyl, quinolinyl, indolyl, isoquinolinyl, and the like.

“Substituted or unsubstituted”: the defined alkyl, aryl, and heteroaryl are unsubstituted or substituted with at least one substituent selected from the group consisting of substituents. The substituents are selected from the following group: halogen atom, alkyl group having 1 to 6 carbon atoms, alkoxy group having 1 to 6 carbon atoms, haloalkyl group having 1 to 6 carbon atoms, haloalkoxy group having 1 to 6 carbon atoms, —CN, alkynyl group having 2 to 6 carbon atoms, alkanoyl group having 1 to 6 carbon atoms, cycloalkyl group having 3 to 7 ring atoms, heteroaryl group, aryl group, aryloxy group having 7 to 10 carbon atoms, arylcarbonyl group, aminocarbonyl group, alkenyl groups having 2 to 5 carbon atoms, alkylthio groups having 1 to 6 carbon atoms, aminosulfinyl groups, aminosulfonyl groups, hydroxyl groups, —SF₅, hydroxyalkyl groups having 1 to 4 carbon atoms, nitro groups, amino groups, carboxyl groups, alkoxycarbonyl groups having 2 to 5 carbon atoms, alkoxyalkyl groups having 1 to 4 carbon atoms, alkylsulfonyl groups having 1 to 4 carbon atoms, alkanoylamino groups having 1 to 4 carbon atoms, alkanoyl (alkyl) amino groups having 1 to 6 carbon atoms, alkanoylamino alkyl groups having 1 to 6 carbon atoms in the alkanoyl and alkyl moieties, alkylsulfonylamino alkyl groups having 1 to 6 carbon atoms in the alkanoyl group and each alkyl moiety, alkylsulfonamido groups having from 1 to 4 carbon atoms, monoalkylaminocarbonyl or dialkylaminocarbonyl groups having from 1 to 6 carbon atoms, monoalkylaminosulfinyl or dialkylamino sulfinyl groups having from 1 to 6 carbon atoms, monoalkylaminosulfonyl groups having from 1 to 6 carbon atoms, or dialkylamino sulfonyl groups, an aminoalkyl group having 1 to 4 carbon atoms, a monoalkylamine group having 1 to 6 carbon atoms, or a dialkylamine group, an aralkyl group having from 1 to 6 carbon atoms in each alkyl moiety, a heteroaralkyl group having from 1 to 4 carbon atoms in the alkyl moiety, a heteroarylalkoxy group having from 1 to 4 carbon atoms in the alkoxy moiety, and an alkylsulfonamido group having from 1 to 4 carbon atoms.

As used herein, the term “heterocyclic” or “heterocyclic” or “heterocycloalkyl” or “heterocyclyl” refers to a saturated, partially saturated or unsaturated group (but not aromatic) having a monocyclic or fused ring (including a bridged ring system and a spiro ring system, with 1 to 10 carbon atoms in the said ring and 1 to 4 heteroatoms selected from nitrogen, sulfur or oxygen, in a fused ring system, one or more rings may be cycloalkyl, aryl, or heteroaryl as long as the connection point is in the non-aromatic moiety. In some embodiments, the nitrogen atoms and/or sulfur atoms of the heterocyclic group are optionally oxidized to provide N-oxide, sulfinyl, and sulfonyl moieties. Examples of “heterocyclyl” and their Condensed analogs include pyrrolidinyl, piperidinyl, piperazinyl, imidazolidinyl, 2,3-dihydrofiran(2,3-b)pyridyl, benzoxazinyl, tetrahydroquinolinyl, tetrahydroisoquinolinyl, indolyl, and the like. The term also includes non-aromatic partially unsaturated monocyclic rings, such as 2- or 4-pyridone or N-substituted-(1H,3H)-pyrimidine-2,4-dione (N-substituted uracil) linked by nitrogen atoms.

As used herein, the term “substituted heterocyclic” or “substituted heterocycloalkyl” or “substituted heterocyclyl” refers to a heterocyclic group substituted with 1 to 5 (eg, 1 to 3) substituents that are identical to the substituents defined by the substituted cycloalkyl.

Unless otherwise stated, the term “halogenated” or “halogen” itself or as part of another substituent refers to fluorine, chlorine, bromine, or iodine atoms. In addition, the term “haloalkyl” refers to a monohaloalkyl group and a polyhaloalkyl group. For example, the term “halogenated (C₁-C₆) alkyl” refers to, but is not limited to, trifluoromethyl, 2,2,2-trifluoroethyl, pentafluoroethyl, 4-chlorobutyl, 3-bromopropyl, and the like.

Optical Isomer-Diastereomer-Geometric Isomer-Tautomer:

The compound of formula (I) contains one or more asymmetric centers, and thus can be used as racemates and racemic mixtures, single enantiomer, diastereomeric mixtures, and single diastereomer. The present disclosure should comprise all of these isomeric forms of the compound of formula (I).

Some of the compounds described herein contain olefinic double bonds, unless stated otherwise, refer to two geometric isomers, including E and Z.

Some compounds of the present disclosure may contain one or more ring systems, so cis- and trans-isomers may be present. The present disclosure is intended to encompass all such cis- and trans-isomers.

Some of the compounds described herein may have different sites connected to hydrogen atoms, referred to as tautomers. Such an example may be a ketone and its enol form known as a ketone-enol tautomer. Single tautomer and mixtures thereof are included in the compounds of the present disclosure.

The compounds of the present invention can be separated into diastereoisomeric pairs of enantiomers, such as by fractional crystallization from a suitable solvent, such as methanol or ethyl acetate, or mixtures thereof. A pair of such enantiomers can be separated into individual stereoisomers by conventional methods, such as using optically active amines or acids as resolution reagents or in chiral HPLC columns.

Alternatively, any enantiomers of the compounds of the present invention can be obtained by stereotactic synthesis using optically pure feedstocks or reagents of known configurations.

A stable isotope-labeled analog: one or more protons in the compound of the present disclosure may be replaced with deuterium atoms, thereby providing a deuterated analog with improved pharmacological activity.

### Salts and Dosage Forms

It should be understood that the compounds of the present invention, as used herein, also include pharmaceutically acceptable salts.

### EXAMPLES

The information provided by the present disclosure is only intended to assist the reader in understanding. Neither the information provided nor the cited references is an admission to the prior art of the present invention. Each reference cited is incorporated herein in its entirety and for any purpose.

All documents mentioned in the present disclosure are incorporated herein by reference as if each document is individually incorporated by reference. In addition, it should be understood that after reading the above teaching content of the present disclosure, those skilled in the art may make various changes or modifications to this application, and these equivalent forms also fall within the scope defined by the appended claims of the present disclosure.

The present invention is further described below with reference to specific embodiments. It should be understood that the following description is only the most preferred embodiments of the present invention, and should not be considered as a limitation on the protection scope of the present invention. On the basis of fully understanding the present disclosure, the experimental methods without specific conditions in the following embodiments are usually made according to conventional conditions or according to conditions suggested by manufacturers, and those skilled in the art can make non-essential changes to the technical solutions of the present disclosure, and such modifications should be regarded as included in the protection scope of the present disclosure.

Unless otherwise specified, the raw materials and reagents used in the following embodiments are all commercially available commodities, or may be prepared by a known method.

The present application has the following definitions:

### Symbol or Unit:

IC₅₀: half inhibitory concentration refers to the concentration at half the maximum inhibitory effect is reached.

### Example 1

### N-(1-((2-amino-5-bromopyridin-3-yl)oxy)-2,3-dihydro-1H-inden-4-yl)-3-methoxybenzamide

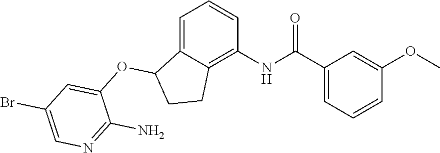 [SMILES: COc1cccc(C(=O)Nc2cccc3c2CCC3Oc2cc(Br)cnc2N)c1]

### N-(1-((2-amino-5-(1-methyl-1H-pyrazol-4-yl)pyridin-3-yl)oxy)-2,3-dihydro-1H-inden-4-yl)-3-methoxybenzamide

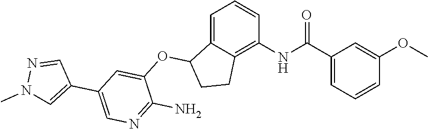

Compound 1 was prepared by the following steps:

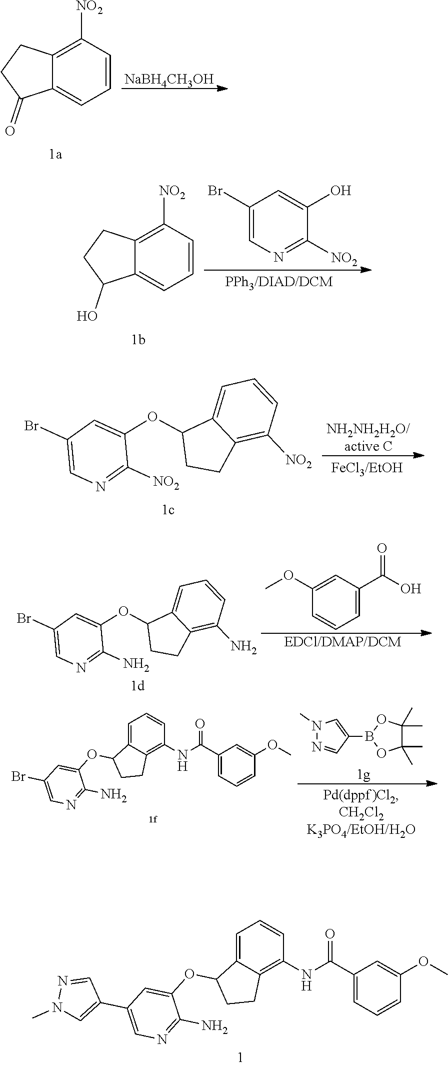

Step 1: To a solution of 4-nitro-2, 3-dihydro-1H-inden-1-one 1a (1.77 g, 10.0 mmol) in methanol (20 mL) was added NaBH₄ (454 mg, 12 mmol) at 0° C. The resulting mixture was kept at 0° C. for 10 min. TLC showed the reaction was complete. The reaction mixture was concentrated in vacuum and purified on an ISCO chromatography to give product 1b as a solid (1.68 g, yield: 93.9%). ESI-MS (m/z): 162.1 [M−17]⁺.

Step 2: To a solution of 4-nitro-2, 3-dihydro-1H-inden-1-ol (1b) (1.68 g, 9.39 mmol), 5-bromo-2-nitropyridin-3-ol (2.47 g, 11.27 mmol) and PPh₃ (3.69 g, 14.09 mmol) in CH₂Cl₂ (20 mL) was added dropwise DIAD (2.85 g, 14.09 mmol) at −10° C. The resulting mixture was kept at −10° C. for 3 h. TLC showed the reaction was complete. A large amount of solid precipitated during the reaction. The solid was filtered, and the filter cake was washed with CH₂Cl₂ and dried to give product as a solid (2.05 g, yield: 57.6%). ESI-MS (m/z): 378.0 [M−1]⁻, 380.0 [M−1]⁻.

Step 3: To a solution of 1c (1.14 g, 3 mmol), activated carbon (230 mg), and FeCl₃ (98 mg, 0.6 mmol) in ethanol (20 mL) heated to reflux was added hydrazine hydrate (1.88 g, 30 mmol). After the reaction mixture was refluxed for 30 min, TLC showed the reaction was complete. The resulting mixture was cooled to room temperature and filtered. The filtrate was concentrated in vacuum and purified on an ISCO chromatography to give product as a solid (0.82 g, yield: 85.4%). ESI-MS (m/z): 320.0 [M+1]⁺, 322.0 [M+1]⁺.

Step 4: A solution of 1d (320 mg, 1 mmol), 3-methoxybenzoic acid (183 mg, 1.2 mmol), EDCI (289 mg, 1.5 mmol) and DMAP (13 mg, 0.1 mmol) in CH₂Cl₂ (1 mL) was stirred overnight at room temperature. TLC showed the reaction was complete. The resulting mixture was concentrated in vacuum and purified on an ISCO chromatography to give product as a solid (320 mg, yield: 70.5%). ESI-MS (m/z): 454.1 [M+1]⁺, 456.1 [M+1]⁺. ¹H NMR (62 MHz, DMSO-d₆) δ 10.00 (s, 1H), 7.64-7.09 (m, 9H), 6.02-5.84 (t, 1H), 5.75 (s, 2H), 3.84 (s, 3H), 3.20-2.53 (m, 3H), 2.30-1.90 (m, 1H).

Step 5: A solution of 1f (272 mg, 0.6 mmol), 1g (175 mg, 0.84 mmol), K₃PO₄ (382 mg, 1.8 mmol), and Pd(dppf)Cl₂CH₂·Cl₂ (49 mg, 0.06 mmol) in a mixture of solvent ethanol (2 mL) and water (1 mL) was degassed under vacuum and purged with N₂ three times, and then stirred at 90° C. for 30 min. TLC showed the reaction was complete. The resulting mixture was concentrated in vacuum and purified on an ISCO chromatography to give product as a solid (168 mg, yield: 61.5%). ESI-MS (m/z): 456.2 [M+1]⁺. ¹H NMR (62 MHz, acetone-d₆) δ 9.14 (s, 1H), 7.88-7.03 (n, 11H), 6.08-5.90 (m, 1H), 5.11 (s, 2H), 3.88 (s, 6H), 3.7-2.89 (m, 2H), 2.76-2.21 (s, 2H).

### Example 2

The preparation of compounds 2a-2c refers to the synthesis of compound 1 in example 1.

TABLE 1

| | | MS | |
|---|---|---|---|
| Cpds | Structure | [M + 1]⁺ | ¹H NMR |
| 2a | 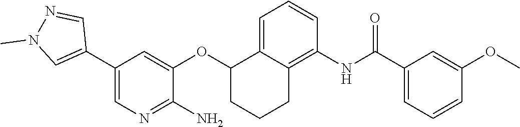 | 470.2 | ¹H NMR (62 MHz, acetone-d₆) δ 9.01 (s, 1H), 7.87-7.04 (m, 11H), 5.68- 5.59 (m, 1H), 5.10 (s, 2H), 3.89 (s, 6H), 2.98-2.85 (m, 4H), 2.01-1.68 (m, 2H). |
| 2b | 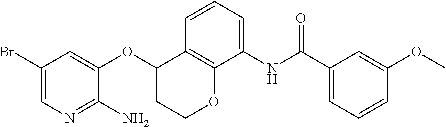 [SMILES: COc1cccc(C(=O)Nc2cccc3c2OCCC3Oc2cc(Br)cnc2N)c1] | 470.1 | ¹H NMR (62 MHz, DMSO-d₆) δ 9.43 (s, 1H), 7.77-6.71 (m, 9H), 5.84 (s, 2H), 5.67-5.61 (m, 1H), 4.43-4.29 (m, 2H), 3.84 (s, 3H), 2.27-1.71 (m, 2H). |
| 2c | 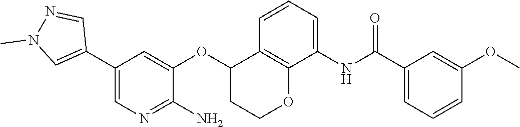 | 472.2 | ¹H NMR (62 MHz, DMSO-d₆) δ 9.45 (s, 1H), 8.05-6.82 (m, 11H), 5.76- 5.60 (m, 1H), 5.52 (s, 2H), 4.49-4.33 (m, 2H), 3.86 (s, 6H), 2.28-2.00 (m, 2H). |

The steps for the synthesis of 3a-3j refer to the synthesis of compound 1 of Example 1, where the chiral center was constructed from 1a by the Corey-Bakshi-Shibata reaction:

(S)-4-nitro-2, 3-dihydro-1H-inden-1-ol (3ab): A solution of 4-nitro-2,3-dihydro-1H-inden-1-one 1a (1.77 g, 10 mmol) in THF (20 mL) was added dropwise into a solution of DEANB (2 mL, 10.4 mmol) and (R)-2-methyl-CBS-oxazaborolane (R—CBS) (0.5 mL, 0.5 mmol) in TH (4 mL) at 0° C. under N₂ during a period of 3 h. The reaction mixture was kept at 0° C. for 10 min and TLC showed the reaction was complete. The reaction mixture was quenched by the addition of methanol, and then concentrated in vacuum. The residue was purified on an ISCO chromatography to give product as a solid (1.55 g, yield: 86.6%). ESI-MS (m/z): 162.1 [M−17]⁺.

(R)-4-nitro-2, 3-dihydro-1H-inden-1-ol (3bb): A solution of 4-nitro-2,3-dihydro-1H-inden-1-one 1a (1.77 g, 10 mmol) in THF (20 mL) was added dropwise into a solution of DEANB (2 mL, 10.4 mmol) and (S)-2-methyl-CBS-oxazaborolane (S—CBS) (0.5 mL, 0.5 mmol) in THF (4 mL) at 0° C. under N₂ during a period of 3 h. The reaction mixture was kept at 0° C. for 10 min and TLC showed the reaction was complete. The reaction mixture was quenched by the addition of methanol, and then concentrated in vacuum. The residue was purified on an ISCO chromatography to give product as a solid (1.58 g, yield: 88.3%). ESI-MS (m/z): 162.1 [M−17]⁺.

TABLE 2

| | | MS | |
|---|---|---|---|
| | | [M + | |
| Cpds | Structure | 1]⁺ | ¹H NMR |
| 3a | 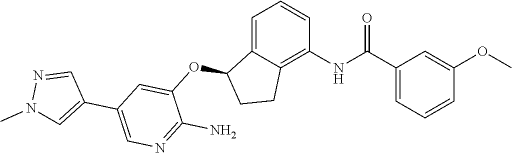 | 456.2 | ¹H NMR (62 MHz, acetone-d₆) δ 9.14 (s, 1H), 7.89-7.04 (m, 11H), 6.08-5.91 (m, 1H), 5.17 (s, 2H), 3.88 (s, 6H), 3.25-2.89 (m, 2H), 2.79-2.22 (m, 2H). |
| 3b | 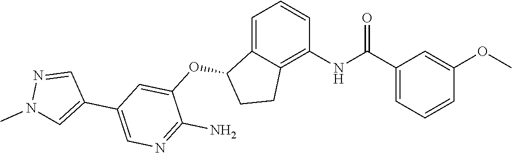 | 456.2 | ¹H NMR (62 MHz, acetone-d₆) δ 9.15 (s, 1H), 7.89-7.04 (m, 11H), 6.08-5.91 (m, 1H), 5.13 (s, 2H), 3.89 (s, 6H), 3.27-2.89 (m, 2H), 2.77-2.22 (m, 2H). |
| 3c | 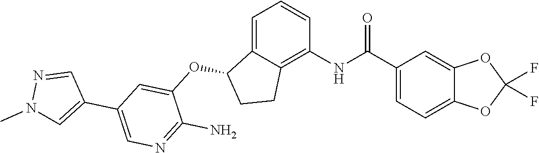 [SMILES: Cn1cc(-c2cnc(N)c(O[C@H]3CCc4c(NC(=O)c5ccc6c(c5)OC(F)(F)O6)cccc43)c2)cn1] | 506.2 | ¹H NMR (62 MHz, acetone-d₆) δ 9.29 (s, 1H), 8.04-7.26 (m, 10H), 6.08-5.91 (m, 1H), 5.15 (s, 2H), 3.89 (s, 3H), 3.26-2.99 (m, 2H), 2.77-2.21 (m, 2H). |
| 3d | 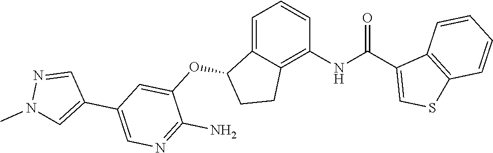 [SMILES: Cn1cc(-c2cnc(N)c(O[C@H]3CCc4c(NC(=O)c5csc6ccccc56)cccc43)c2)cn1] | 482.2 | ¹H NMR (62 MHz, acetone-d₆) δ 9.21 (s, 1H), 8.64-8.48 (m, 2H), 8.12-7.19 (m, 10H), 6.09-5.92 (m, 1H), 5.10 (s, 2H), 3.89 (s, 3H), 3.30-2.89 (m, 2H), 2.67- 2.23 (m, 2H). |
| 3e | 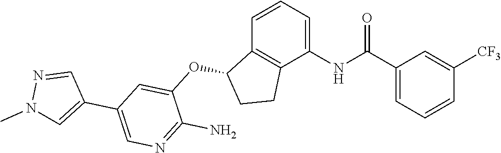 [SMILES: Cn1cc(-c2cnc(N)c(O[C@H]3CCc4c(NC(=O)c5cccc(C(F)(F)F)c5)cccc43)c2)cn1] | 494.2 | ¹H NMR (62 MHz, acetone-d₆) δ 9.52 (s, 1H), 8.45-7.97 (m, 2H), 7.95-7.85 (m, 2H), 7.83 (s, 1H), 7.78-7.67 (m, 2H), 7.66-7.55 (m, 1H), 7.53-7.40 (m, 1H), 7.36 (s, 1H), 7.29 (d, J = 2.4 Hz, 1H), 6.00 (m, 1H), 5.13 (s, 2H), 3.89 (s, 3H), 3.61-2.17 (m, 4H). |
| 3f | 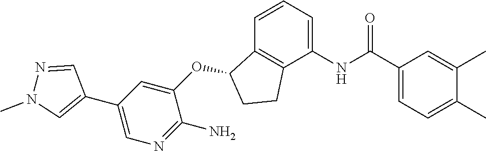 | 454.2 | ¹H NMR (62 MHz, acetone-d₆) δ 9.05 (s, 1H), 7.99-7.85 (m, 1H), 7.84-7.76 (m, 2H), 7.75-7.64 (m, 2H), 7.63-7.41 (m, 2H), 7.32 (s, 2H), 7.27-7.13 (m, 1H), 5.98 (t, J = 5.4 Hz, 1H), 5.23 (s, 2H), 3.88 (s, 3H), 3.66 (s, 2H), 3.44-2.87 (m, 2H), 2.86-2.42 (m, 1H), 2.32 (s, 6H). |
| 3g | 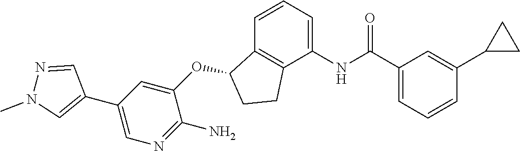 [SMILES: Cn1cc(-c2cnc(N)c(O[C@H]3CCc4c(NC(=O)c5cccc(C6CC6)c5)cccc43)c2)cn1] | 466.2 | ¹H NMR (62 MHz, acetone-d₆) δ 9.16 (s, 1H), 8.00-7.92 (m, 2H), 7.87 (s, 1H), 7.85- 7.55 (m, 4H), 7.52 (d, J = 1.9 Hz, 1H), 7.47-7.39 (m, 1H), 7.35-7.24 (m, 1H), 5.99 (t, J = 5.4 Hz, 1H), 5.09 (d, 2H), 3.89 (s, 3H), 3.42-2.72 (m, 2H), 2.72-2.17 (m, 2H), 1.24-0.58 (m, 5H). |
| 3h | 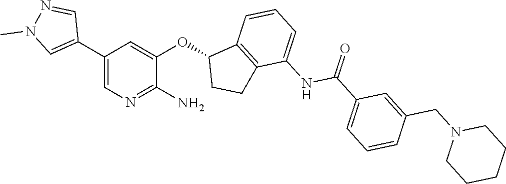 [SMILES: Cn1cc(-c2cnc(N)c(O[C@H]3CCc4c(NC(=O)c5cccc(CN6CCCCC6)c5)cccc43)c2)cn1] | 523.3 | ¹H NMR (62 MHz, acetone-d₆) δ 9.24 (s, 1H), 8.12-7.98 (m, 1H), 7.98-7.80 (m, 3H), 7.80-7.62 (m, 2H), 7.60-7.40 (m, 3H), 7.34 (s, 1H), 7.30-7.21 (m, 1H), 6.16-5.81 (m, 1H), 5.10 (s, 2H), 3.89 (s, 3H), 3.58 (s, H), 3.37-2.96 (m, 2H), 2.70-2.18 (m, 6H), 1.73-1.38 (m, 6H). |
| 3i | 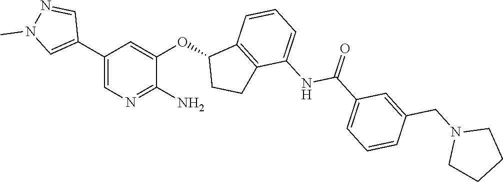 [SMILES: Cn1cc(-c2cnc(N)c(O[C@H]3CCc4c(NC(=O)c5cccc(CN6CCCC6)c5)cccc43)c2)cn1] | 509.3 | ¹H NMR (62 MHz, acetone-d₆) δ 9.27 (s, 1H), 8.21-8.04 (m, 1H), 8.04-7.80 (m, 3H), 7.80-7.65 (m, 2H), 7.63-7.41 (m, 2H), 7.38-7.18 (m, 2H), 5.99 (t, 1H), 5.10 (s, 2H), 3.89 (s, 3H), 3.81 (s, 2H), 3.36-2.97 (m, 3H), 2.74-2.18 (m, 6H), 1.93-1.64 (m, 4H). |
| 3j | 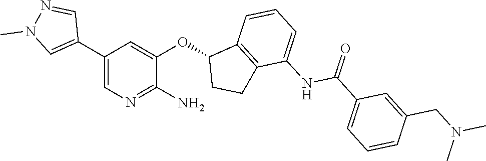 | 483.3 | ¹H NMR (62 MHz, acetone-d₆) δ 9.21 (s, 1H), 8.08-7.94 (m, 1H), 7.93-7.79 (m, 3H), 7.78-7.65 (m, 2H), 7.64-7.38 (m, 3H), 7.37-7.08 (m, 2H), 5.99 (t, J = 5.4 Hz, 1H), 5.20-4.80 (m, 1H), 3.89 (s, 3H), 3.57-3.7 (m, 2H), 3.4-2.94 (m, 2H), 2.71- 2.30 (m, 1H), 2.22 (s, 6H), 1.32-0.91 (m, 1H). |

### Example 4

### (S)-1-((2-amino-5-(1-methyl-1H-pyrazol-4-yl)pyridin-3-yl)oxy)-N-(3-cyclopropylphenyl)-2,3-dihydro-1H-indene-4-carboxamide

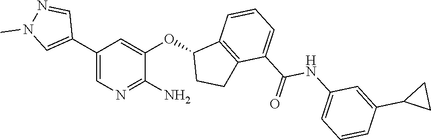 [SMILES: Cn1cc(-c2cnc(N)c(O[C@H]3CCc4c(C(=O)Nc5cccc(C6CC6)c5)cccc43)c2)cn1]

Compound 4 was prepared by the following steps:

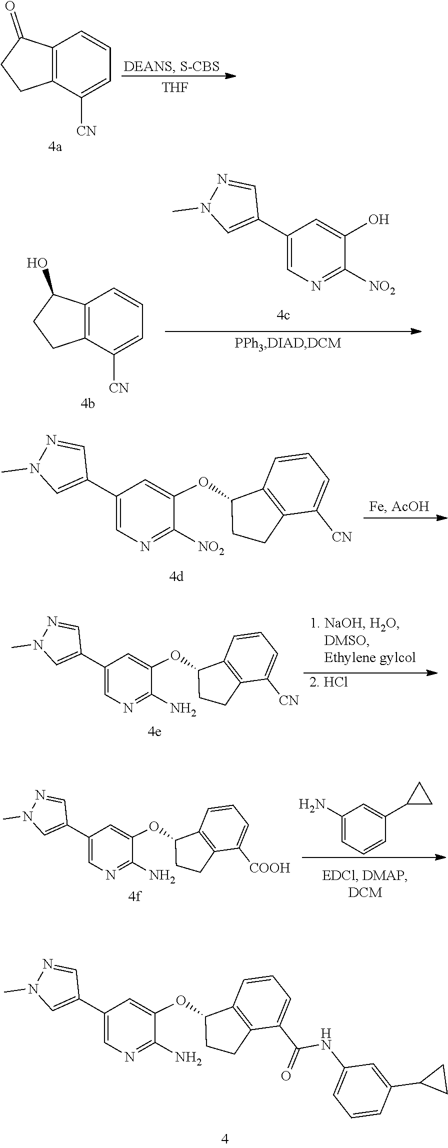

Step 1: A solution of 4a (3.0 g, 19.1 mmol) in THF (20 mL) was added dropwise into a solution of DEANB (3.8 mL, 19.1 mmol) and S—CBS (1.0 mL, 1.0 mmol) in THF (5 mL) at −5-0° C. under N₂ during a period of 3 h. The reaction mixture was kept at 0° C. for 30 min and TLC showed the reaction was complete. The reaction mixture was quenched by the addition of methanol, and then concentrated in vacuum. The residue was diluted with ethyl acetate, and then washed with dilute H₂SO₄ (10%, w/w), saturated NaHCO₃ solution and brine in turn. The organic layer was dried and concentrated in vacuum. Toluene was added into the residue and then evaporated to dryness to give product as a solid (2.87 g, yield: 94.4%). ESI-MS (m/z): 142.1 [M−17]⁺.

Step 2: To a solution of 4b (1.0 g, 6.4 mmol), 4c (Obtained by Suzuki coupling of 5-bromo-2-nitropyridine-3-phenol and 1 g. Synthesis steps refer to the step 5 of Example 1) (1.54 g, 7.0 mmol) and PPh₃ (2.14 g, 8.2 mmol) in CH₂Cl₂ (40 mL) was added dropwise DIAD (1.90 g, 9.4 mmol) at −15-−10° C. The resulting mixture was kept at −10° C. for 16 h and TLC showed the reaction was complete. A large amount of solid was precipitated. After filtration, the filter cake was washed with CH₂Cl₂ and dried to give product as a solid (1.34 g, yield: 58.9%). ESI-MS (m/z): 362.1 [M+1]⁺.

Step 3: A solution of 4d (361 mg, 1.0 mmol), ferrous powder (280 mg, 5.0 mmol) in CH₃COOH (5 mL) was stirred between 40-45° C. for 20 min and TLC showed the reaction was complete. Water was added to the mixture, and the mixture was extracted with ethyl acetate. The combined organic layers were washed with water, saturated NaHCO₃ solution and brine in turn, dried over Na₂SO₄, filtered and concentrated. The residue was purified on an ISCO chromatography to give product as a solid (323 mg, yield: 97.6%). ESI-MS (m/z): 330.1 [M−1]⁻, 332.1 [M+1]⁺.

Step 4: A round-bottom flask was charged with compound 4e (166 mg, 0.5 mmol), NaOH (0.5 g, 12.5 mmol), H₂O (0.5 g), DMSO (1.0 g) and glycol (0.5 g). The resulting mixture was stirred at 100-105° C. for 16 h and TLC showed the reaction was complete. The mixture was diluted with water and adjusted to pH=5-6 with aqueous HCl, and then extracted with ethyl acetate. The combined organic layers were washed with water and brine in turn, dried over Na₂SO₄ and filtered. After concentration, the residue was purified on an ISCO chromatography to give product as a solid (70 mg, yield: 40.0/%). ESI-MS (m/z): 349.1 [M−1]⁻, 351.1 [M+1]⁺.

Step 5: A solution of 4f (83 mg, 0.22 mmol), 3-cyclopropyl aniline (38.6 mg, 0.29 mmol), EDCI (64.1 mg, 0.33 mmol), and DMAP (2.7 mg, 0.02 mmol) in CH₂Cl₂ (1 mL) was stirred at room temperature for 1.5 h and TLC showed the reaction was complete. The reaction mixture was diluted with CH₂Cl₂ (2 mL), and washed with water (1 mL) twice. The organic layer was concentrated in vacuum and purified on an ISCO chromatography to give product as a solid (30.5 mg, yield: 29.8%). ESI-MS (m/z): 464.2 [M−1]⁻, 466.2 [M+1]⁺. ¹H NMR (62 MHz, acetone-d₆) δ 9.34 (s, 1H), 7.99-7.77 (m, 2H), 7.77-7.64 (m, 2H), 7.64-7.41 (m, 4H), 7.40-7.06 (m, 2H), 7.02-6.67 (m, 1H), 6.15-5.80 (m, 1H), 5.10 (s, 2H), 3.89 (s, 3H), 3.58-3.14 (m, 2H), 2.76-2.20 (m, 2H), 1.95-1.68 (m, 1H), 1.15-0.52 (m, 4H).

### Example 5

The preparation of compounds 5a-5e refers to the synthesis of compound 4 in example 4.

TABLE 3

| | | MS | |
|---|---|---|---|
| | | [M + | |
| Cpds | Structure | 1]⁺ | ¹HNMR |
| 5a | 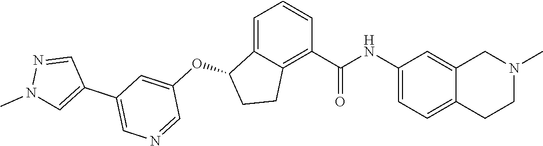 | 495.2 | ¹H NMR (62 MHz, CDCl₃) δ 7.91-7.33 (m, 7H), 7.11 (m, 3H), 5.82 (t, J = 5.3 Hz, 1H), 4.61 (s, 2H), 3.95 (s, 3H), 3.75 (s, 2H), 3.52-3.28 (m, 2H), 3.17-2.70 (m, 4H), 2.70-2.24 (m, 5H). |
| 5b | 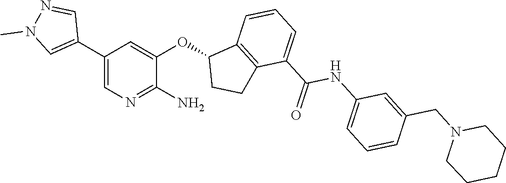 [SMILES: Cn1cc(-c2cnc(N)c(O[C@H]3CCc4c(C(=O)Nc5cccc(CN6CCCCC6)c5)cccc43)c2)cn1] | 523.3 | ¹H NMR (400 MHz, acetone-d₆) δ 9.42 (s, 1H), 7.91 (s, 1H), 7.86 (d, J = 1.8 Hz, 1H), 7.82-7.72 (m, 4H), 7.64 (d, J = 7.5 Hz, 1H), 7.50 (d, J = 1.8 Hz, 1H), 7.39 (t, J = 7.6 Hz, 1H), 7.30 (t, J = 7.7 Hz, 1H), 7.09 (d, J = 7.5 Hz, 1H), 5.99 (dd, J = 6.7, 4.3 Hz, 1H), 5.11 (d, J = 7.8 Hz, 2H), 3.90 (s, 3H), 3.51-3.41 (m, 3H), 3.30-3.19 (m, 1H), 2.76-2.65 (m, 1H), 2.38 (s, 4H), 2.26-2.16 (m, 1H), 1.55 (p, J = 5.5 Hz, 4H), 1.48-1.39 (m, 2H). |
| 5c | 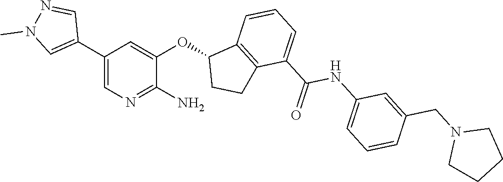 [SMILES: Cn1cc(-c2cnc(N)c(O[C@H]3CCc4c(C(=O)Nc5cccc(CN6CCCC6)c5)cccc43)c2)cn1] | 509.3 | ¹H NMR (400 MHz, acetone-d₆) δ 9.42 (s, 1H), 7.91 (d, J = 0.8 Hz, 1H), 7.86 (d, J = 1.8 Hz, 1H), 7.82 (d, J = 2.1 Hz, 1H), 7.80-7.72 (m, 3H), 7.64 (d, J = 7.5 Hz, 1H), 7.55-7.47 (m, 1H), 7.43-7.34 (m, 1H), 7.34-7.26 (m, 1H), 7.10 (d, J = 7.5 Hz, 1H), 5.99 (dd, J = 6.7, 4.4 Hz, 1H), 5.10 (d, J = 7.9 Hz, 2H), 3.90 (s, 3H), 3.61 (s, 2H), 3.47 (ddd, J = 17.1, 8.6, 5.5 Hz, 1H), 3.30-3.19 (m, 1H), 2.71 (dddd, J = 13.8, 8.5, 6.8, 5.6 Hz, 1H), 2.50 (s, 4H), 2.27-2.16 (m, 1H), 1.75 (p, J = 3.2 Hz, 4H). |
| 5d | 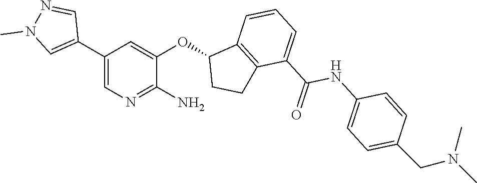 | 483.2 | ¹H NMR (62 MHz, acetone-d₆) δ 9.42 (s, 1H), 7.98-7.66 (m, 6H), 7.65-7.15 (m, 5H), 6.13-5.87 (m, 1H), 5.09 (s, 2H), 3.90 (s, 3H), 3.68-3.05 (m, 4H), 2.78-2.30 (m, 2H), 2.27-2.15 (m, 6H). |
| 5e | 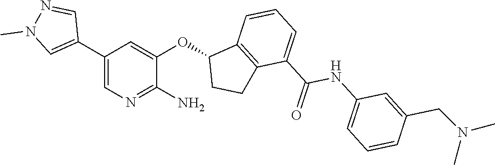 | 483.2 | ¹H NMR (62 MHz, acetone-d₆) δ 9.41 (s, 1H), 7.98-7.65 (m, 6H), 7.65-6.91 (m, 5H), 5.99 (m, 1H), 5.10 (s, 2H), 3.90 (s, 3H), 3.58-3.16 (m, 4H), 2.79-2.28 (m, 2H), 2.20 (s, 6H). |

### Example 6

### (S)—N-(1-((2-amino-5-(1-methyl-1H-pyrazol-4-yl) pyridin-3-yl)amino)-2,3-dihydro-1H-inden-4-yl)-4-fluoro-3-methylbenzamide

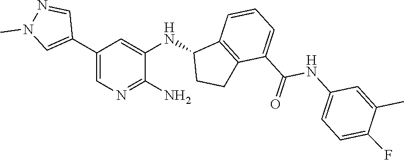

Compound 6 was prepared by the following steps.

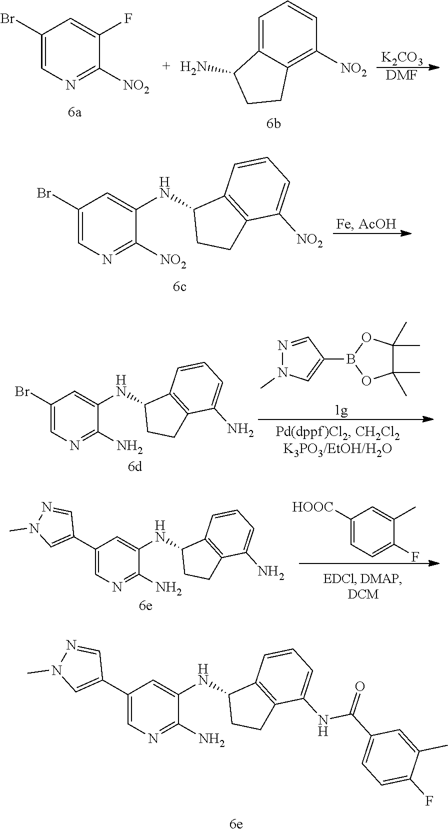

Step 1: A solution of 5-bromo-3-fluoro-2-nitropyridine 6a (221 mg, 1.0 mmol), (1S)-4-nitro-2, 3-dihydro-1H-inden-1-amine 6b (178 mg, 1.0 mmol) and K₂CO₃ (138 mg, 1.0 mmol) in DMF (5.0 mL) was stirred at room temperature for 16 h. TLC showed the reaction was complete. Water was added into the mixture, and the mixture was extracted with ethyl acetate. The combined organic layers were washed with water and brine in turn, dried over Na₂SO₄ and filtered. After concentration, the mixture was purified on an ISCO chromatography to give compound 6c (289 mg, yield: 76.2%). ESI-MS (m/z): 378.0/378.9 [M−1]⁻, 379.0/380.9 [M+1]⁺.

Step 2: To a flask was added 6c (289 mg, 0.8 mmol), ferrous powder (426 mg, 8.0 mmol) and CH₃COOH (2.5 mL). The resulting mixture was stirred at 50-55° C. for 4 h. TLC showed the reaction was complete. Water was added into the mixture, and the mixture was extracted with ethyl acetate. The combined organic lavers were washed with water, saturated NaHCO₃ solution and brine in turn, dried over Na₂SO₄ and filtered. After concentration, the mixture was purified on an ISCO chromatography to give compound 6d (146 mg, yield: 57.2%). ESI-MS (m/z): 317.0/318.9 [M−1]⁻, 319.0/320.9 [M+1]⁺.

Step 3: A mixture of 6d (157 mg, 0.49 mmol), 1g (136 mg, 0.65 mmol), K₃PO₄ (313 mg, 1.48 mmol) and Pd(dppf)Cl₂·CH₂Cl₂ (4 mg, 0.005 mmol) in a mixture of solvent ethanol (0.5 mL) and water (0.25 mL) was degassed under vacuum and purged with N₂ three times. The reaction mixture was stirred at 85˜95° C. for 2 h. TLC showed the reaction was complete. After cooling, the mixture was concentrated in vacuum and purified on an ISCO chromatography to give compound 6e as a solid (133 mg, yield: 84.8%). ESI-MS (m/z): 319.2 [M−1]⁻, 321.2 [M+1]⁺.

Step 4: A solution of 6e (32 mg, 0.1 mmol), 4-fluoro-3-methylbenzoic acid (23.1 mg, 0.15 mmol), EDCI (38.3 mg, 0.2 mmol), and DMAP (1.2 mg, 0.01 mmol) in CH₂Cl₂ (1 mL) was stirred overnight at room temperature. TLC showed the reaction was complete. After concentration, the mixture was purified on an ISCO chromatography to give compound 6 as a solid (20 mg, yield: 43.8%). ESI-MS (m/z): 455.2 [M−1]⁻, 457.2 [M+1]⁺. ¹H NMR (62 MHz, acetone-d₆) δ 9.14 (s, 1H), 8.08-7.76 (m, 3H), 7.75-7.45 (m, 3H), 7.20 (m, 4H), 5.43-4.74 (m, 3H), 4.42 (d, J=7.9 Hz, 1H), 3.87 (s, 3H), 3.06 (d, 2H), 2.79-2.43 (m, 1H), 2.34 (s, 3H), 1.95-1.56 (m, 1H).

### Example 7

### (S)-1-((2-amino-5-chloropyridin-3-yl)oxy)-N-(3-cyclopropylphenyl)-2,3-dihydro-1H-indene-4-carboxamide

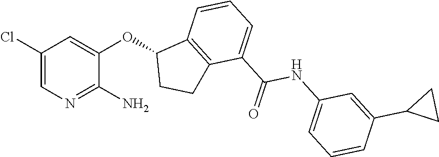 [SMILES: Nc1ncc(Cl)cc1O[C@H]1CCc2c(C(=O)Nc3cccc(C4CC4)c3)cccc21]

Compound 7 was prepared by the following steps:

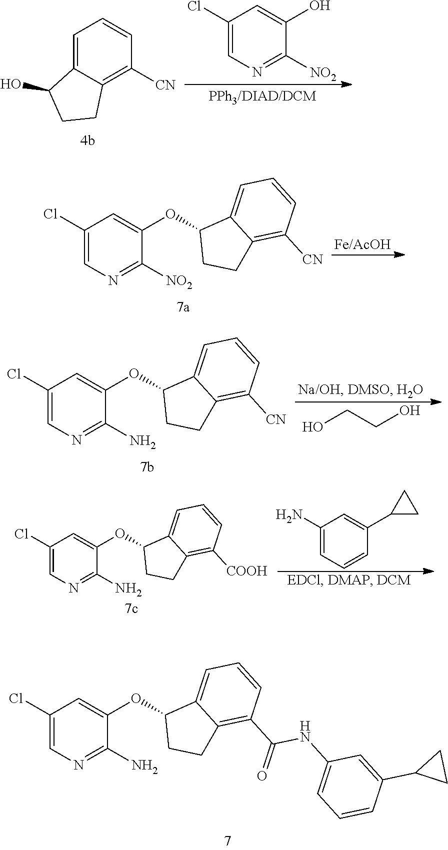

Step 1: To a solution of (1R)-1-hydroxy-2,3-dihydro-1H-indene-4-carbonitrile 4b (318 mg, 2.0 mmol), 5-chloro-2-nitropyridin-3-ol (419 mg, 2.4 mmol) and PPh₃ (682 mg, 2.6 mmol) in CH₂Cl₂ (4 mL) cooled to −10° C. was added dropwise DIAD (526 mg, 2.6 mmol). The resulting mixture was kept at −10° C. for 3 h. TLC showed the reaction was complete. The large amount of solid precipitated during reaction was filtered, and the filter cake was washed with CH₂Cl₂, then dried to give compound 7a as a solid (367 mg, yield: 58.0%). ESI-MS (m/z): 316.7 [M+1]⁺.

Step 2: A mixture of 7a (366.5 mg, 1.2 mmol), ferrous powder (403 mg, 7.2 mmol) in CH₃COOH (6 mL) was heated to 40° C. and stirred for 30 min. TLC showed the reaction was complete. The suspension was cooled to room temperature and filtered. The filtrate was concentrated in vacuum and purified on an ISCO chromatography to give compound 7b as a solid (300 mg, yield: 90.5%). ESI-MS (m/z): 286.7 [M+1]⁺.

Step 3: To a solution of 7b (299 mg, 1.1 mmol) in a mixture of solvent DMSO (2 mL) and glycol (1 mL) was added 50% NaOH solution (1 g, 26 mmol). The mixture was heated at 100° C. overnight. TLC showed the reaction was complete. The mixture was cooled and acidified with HCl (aq.) to pH=5. The resulting precipitate was collected by filtration, and dried to give compound 7c (68 mg, yield: 21%). ESI-MS (m/z): 305.7 [M+1]⁺.

Step 4: A solution of 7c (68 mg, 0.22 mmol), 3-cyclopropyl aniline (36 mg, 0.27 mmol), EDCI (56 mg, 0.29 mmol), and DMAP (5 mg, 0.04 mmol) in CH₂Cl₂ (0.3 mL) was stirred at room temperature overnight. TLC showed the reaction was complete. After concentration, the mixture was purified on an ISCO chromatography to give compound 7 as a solid (42.7 mg, yield: 46.7%). ESI-MS (m/z): 410.9 [M+1]⁺. ¹H NMR (62 MHz, acetone-d₆) δ 9.32 (s, 1H), 7.80-7.65 (m, 1H), 7.64-7.53 (m, 3H), 7.49 (s, 1H), 7.47-7.29 (m, 2H), 7.28-6.98 (m, 1H), 6.96-6.70 (m, 1H), 6.01 (d, 1H), 5.33 (s, 1H), 3.35-2.93 (m, 2H), 3.14-2.90 (m, 1H), 2.66-2.28 (m, 1H), 1.39-0.54 (m, 5H).

### Example 8

### N-(1-((5-bromopyridin-3-yl)amino)-2,3-dihydro-1H-inden-4-yl)-3-methylbenzamide

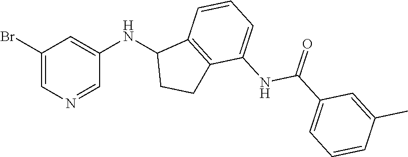 [SMILES: Cc1cccc(C(=O)Nc2cccc3c2CCC3Nc2cncc(Br)c2)c1]

### 3-methyl-N-(1-((5-(1-methyl-1H-pyrazol-4-yl) pyridin-3-yl)amino)-2,3-dihydro-1H-inden-4-yl)benzamide

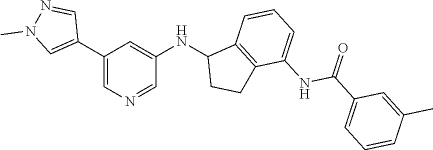

Compound 8d and compound 8 were prepared by the following steps:

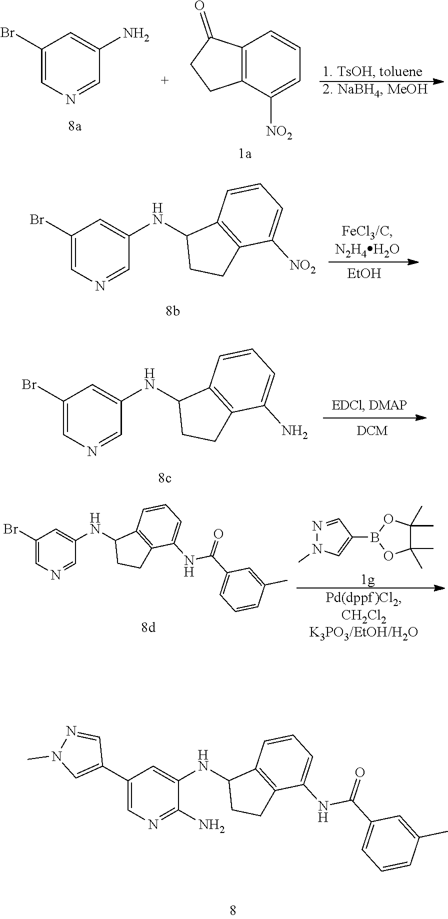

Step 1: A mixture of 5-bromo-3-aminopyridine 8a (541 mg, 3.0 mmol), 4-nitro-2, 3-dihydro-1H-inden-1-one 1a (344 mg, 2.0 mmol) and TsOH (34 mg, 0.2 mmol) in toluene (10 mL) was heated to 120° C. and removed water by Dean-Stark trap. The reaction was continued until TLC showed the reaction was complete. The reaction mixture was cooled to room temperature, concentrated to dryness and purified by column chromatography to give a thick oil. To the solution of the above imine intermediate in methanol (10 mL) cooled to 0° C. was added NaBH₄ (780 mg, 20.0 mmol) in portions. The resulting mixture was allowed to warm to room temperature and stirred until TLC showed the reaction was complete. After concentration, the mixture was purified by column chromatography to give compound 8b (351 mg, yield: 52.0%). ESI-MS (m/z): 334.1 [M+H]⁺.

Step 2: To a mixture of 8b (334 mg, 1.0 mmol), FeCl₃ (16 mg, 0.1 mmol) and activated carbon (33 mg) in ethanol (5 mL) heated to reflux was added dropwise 80% hydrazine hydrate (320 mg, 5.0 mmol). After the drop was finished, the reaction mixture was refluxed until TLC showed the reaction was complete. After the mixture was cooled to room temperature, the mixture was filtered. The filter cake was washed with a mixture of solvent CH₂Cl₂ and MeOH (v/v=1:1). The combined filtrates were concentrated to dryness to give the crude product, which was triturated with ethanol (5 mL) to give compound 8c (250 mg, yield: 83.0%). ESI-MS (m/z): 304.1 [M+H]⁺.

Step 3: A solution of 8c (250 mg, 0.8 mmol), 3-methylbenzoic acid (136 mg, 1.0 mmol), EDCI (192 mg, 1.0 mmol), and DMAP (12 mg, 1.0 mmol) in CH₂Cl₂ (0.3 mL) was stirred at room temperature until TLC showed the reaction was complete. The reaction mixture was concentrated to dryness and purified by column chromatography to give compound 8d (240 mg, yield: 71.0%). ESI-MS (m/z): 422.0 [M+H]⁺. ¹H NMR (62 MHz, acetone-d₆) δ 9.87 (s, 1H), 8.00 (d, J=2.4 Hz, 1H), 7.88-7.56 (m, 3H), 7.48-7.13 (m, 5H), 7.07 (d, J=2.9 Hz, 1H), 6.52 (d, J=8.2 Hz, 1H), 5.19-4.87 (m, 1H), 2.86-2.69 (m, 2H), 2.68-2.48 (m, 1H), 2.32 (s, 3H), 1.81-1.48 (m, 1H).

Step 4: To a mixture of 8d (107 mg, 0.3 mmol), 1g (104 mg, 0.5 mmol), KaPO₄ (212 mg, 1.0) mmol) in a mixture of solvent ethanol (1 mL) and water (0.5 mL) was added Pd(dppf)Cl₂·CH₂Cl₂ (3 mg, 0.003 mmol). The resulting mixture was degassed with N₂ for three times, then heated to reflux and stirred until TLC showed the reaction was complete. After natural cooling to room temperature, the separated organic layer was concentrated to dryness and purified by column chromatography to give the crude product, which was triturated with a mixture of solvent CH₂Cl₂ and diethyl ether (v/v=1:1) to afford compound 8 (33 mg, yield: 8%). ESI-MS (m/z): 424.2 [M+H]⁺. ¹H NMR (62 MHz, acetone-d₆) δ 9.96 (s, 1H), 8.16 (s, 1H), 8.05-7.92 (m, 2H), 7.86-7.71 (m, 3H), 7.51-7.10 (m, 6H), 6.25 (d, J=8.4 Hz, 1H), 5.21-5.08 (m, 1H), 3.87 (s, 3H), 2.90-2.79 (m, 2H), 2.64-2.44 (m, 1H), 2.40 (s, 3H), 1.99-1.81 (m, 1H).

### Example 9

### 2,2-difluoro-N-(1-((5-(1-methyl-1H-pyrazol-4-yl) pyridin-3-yl)amino)-2,3-dihydro-1H-inden-4-yl)benzo[d][1,3]dioxole-5-carboxamide

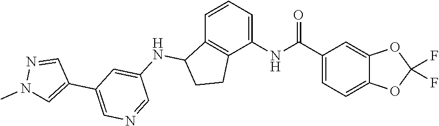 [SMILES: Cn1cc(-c2cncc(NC3CCc4c(NC(=O)c5ccc6c(c5)OC(F)(F)O6)cccc43)c2)cn1]

Compound 9 was prepared by the following steps:

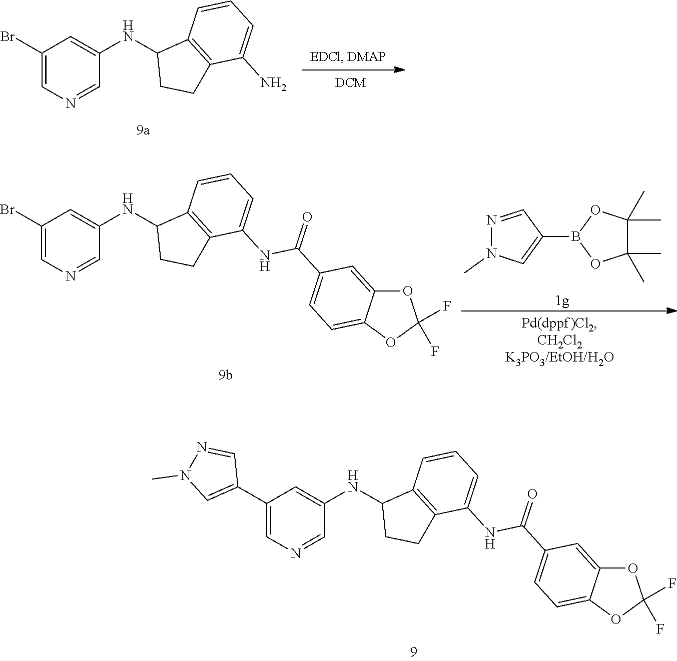

The preparation of compound 9 refers to the synthesis of compound 8 in example 8.

¹H NMR (62 MHz, DMSO-d₆) δ 9.90 (s, 1H), 8.03-7.65 (m, 6H), 7.46-6.94 (m, 5H), 6.16-6.03 (m, 1H), 5.17-4.80 (m, 1H), 3.71 (s, 3H), 3.71-3.40 (m, 3H), 1.82-1.43 (m, 1H).

### Example 10

### (S)-1-((5-bromopyridin-3-yl)amino)-N-(3-cyclopropylphenyl)-2,3-dihydro-1H-indene-4-carboxamide

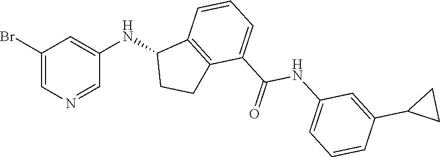 [SMILES: O=C(Nc1cccc(C2CC2)c1)c1cccc2c1CC[C@@H]2Nc1cncc(Br)c1]

Compound 10 was prepared by the following steps:

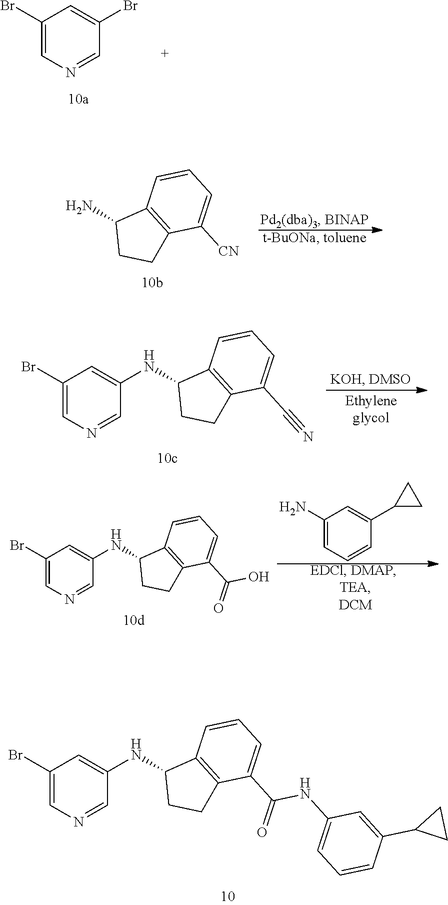

Step 1: To a solution of 3,5-dibromopyridine 10a (2.83 g, 12.0 mmol), (1S)-1-amino-2,3-dihydro-1H-indene-4-carbonitrile 10b (1.58 g, 10.0 mmol), sodium tert-butoxide (2.88 g, 30.0 mmol) and BINAP (1.25 g, 2.0 mmol) in toluene (10 mL) was added Pd₂(dba)₃ (458 mg, 0.5 mmol). The resulting mixture was degassed with N₂ for three times, then heated to 90° C. and stirred until TLC showed the reaction was complete. After natural cooling to room temperature, the reaction mixture was diluted with ethyl acetate (50 mL), washed with water (50 mL), and dried over Na₂SO₄. After concentration to dryness, the residue was purified by column chromatography to give compound 10c (2.13 g, yield: 68.0%). ESI-MS (m/z): 314.2 [M+H]⁺.

Step 2: To a solution of 10c (1.90 g, 6.0 mmol) in a mixture of solvent DMSO (10 mL) and glycol (6 mL) was added water (5 mL) and KOH (2.5 g). The mixture was heated to 110° C. and stirred until TLC showed the reaction was complete. After natural cooling to room temperature, the reaction mixture was diluted with water (20 mL), adjusted to pH=6-7 with 1N HCl (aq.) and extracted with ethyl acetate (50 mL). The organic layer was washed with brine (50 mL), dried over Na₂SO₄, and concentrated to dryness to give crude product 10d (1.66 g, yield: 83.0%). ESI-MS (m/z): 333.2 [M+H]⁺.

Step 3: To a solution of 10d (101 mg, 0.3 mmol), 3-cyclopropyl aniline (66 mg, 0.5 mmol), DMAP (4 mg, 0.03 mmol) and triethylamine (66 mg, 0.6 mmol) in CH₂Cl₂ (0.5 mL) was added EDCI (96 mg, 0.5 mmol). The reaction mixture was stirred at room temperature until TLC showed the reaction was complete, then concentrated to dryness and purified by column chromatography to give compound 10 (67 mg, yield: 51.0%). ESI-MS (m/z): 448.1 [M+H]⁺. ¹H NMR (400 MHz, DMSO-d₆) δ 10.13 (s, 1H), 8.09 (s, 1H), 7.85 (s, 1H), 7.62 (d, J=7.5 Hz, 1H), 7.54 (d, J=8.1 Hz, 1H), 7.49 (s, 1H), 7.44 (d, J=7.5 Hz, 1H), 7.38-7.33 (m, 2H), 7.21 (t, J=7.8 Hz, 1H), 6.83 (d, J=7.6 Hz, 1H), 6.61 (d, J=7.6 Hz, 1H), 5.12-5.06 (m, 1H), 3.25-3.17 (m, 1H), 3.08-3.00 (m, 1H), 2.62-2.54 (m, 1H), 1.94-1.87 (m, 1H), 1.85-1.71 (m, 1H), 1.04-0.92 (m, 2H), 0.71-0.60 (m, 2H).

### Example 11

### (S)—N-(1-((5-bromopyridin-3-yl)amino)-2,3-dihydro-1H-inden-4-yl)-3-cyclopropylbenzamide

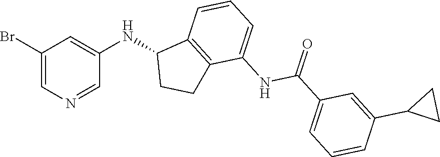 [SMILES: O=C(Nc1cccc2c1CC[C@@H]2Nc1cncc(Br)c1)c1cccc(C2CC2)c1]

Compound 11 was prepared by the following steps:

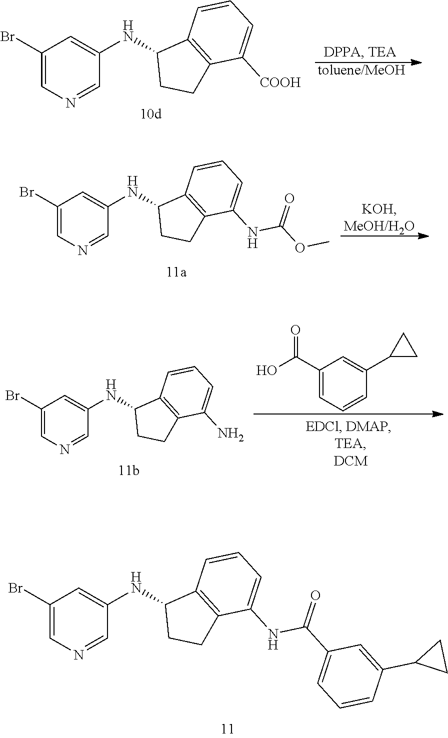

Step 1: To a solution of 10d (333 mg, 1.0 mmol) in toluene (2 mL) was added DPPA (413 mg, 1.5 mmol) and triethylamine (202 mg, 2.0 mmol). The reaction mixture was stirred at room temperature for 24 h, then methanol (3 mL) was added to the above mixture. The resulting mixture was heated to 60° C. and stirred until TLC showed the reaction was complete. After natural cooling to room temperature, the mixture was concentrated to dryness and purified by column chromatography to give compound 11a (275 mg, yield: 76.0%). ESI-MS (m/z): 362.2 [M+H]⁺.

Step 2: To a solution of 11a (275 mg, 0.8 mmol) in methanol (1 mL) was added water (0.5 mL) and KOH (250 mg). The reaction mixture was heated to reflux and stirred until TLC showed the reaction was complete. After natural cooling to room temperature, the mixture was concentrated to dryness and purified by column chromatography to give compound 11b as a solid (159 mg, yield: 69.0%). ESI-MS (m/z): 304.2 [M+H]⁺.

Step 3: To a solution of 11b (100 mg, 0.3 mmol), 3-cyclopropylbenzoic acid (81 mg, 0.5 mmol), DMAP (4 mg, 0.03 mmol) in CH₂Cl₂ (0.5 mL) was added EDCI (96 mg, 0.5 mmol). The reaction mixture was stirred at room temperature until TLC showed the reaction was complete. The resulting mixture was concentrated to dryness and purified by column chromatography to give compound 11 (86 mg, yield: 64.0%). ESI-MS (m/z): 448.1 [M+H]⁺.

¹H NMR (400 MHz, DMSO-d₆) δ 9.97 (s, 1H), 8.09 (s, 1H), 7.84 (s, 1H), 7.73 (d, J=7.4 Hz, 1H), 7.65 (s, 1H), 7.44-7.35 (m, 2H), 7.35-7.29 (m, 2H), 7.25 (t, J=7.6 Hz, 1H), 7.14 (d, J=7.4 Hz, 1H), 6.63 (d, J=8.2 Hz, 1H), 5.13-5.07 (m, 1H), 3.01-2.74 (m, 2H), 2.61-2.52 (m, 1H), 2.06-1.99 (m, 1H), 1.79-1.70 (m, 1H), 1.02-0.98 (m, 2H), 0.80-0.76 (m, 2H).

### Example 12

### (S)-3-methyl-N-(1-((5-(1-methyl-1H-pyrazol-4-yl) pyridin-3-yl)amino)-2,3-dihydro-1H-inden-4-yl)benzamide

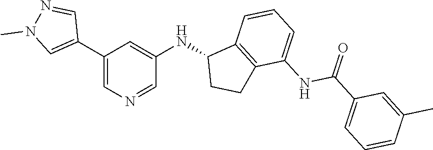

Compound 12 was prepared by the following steps:

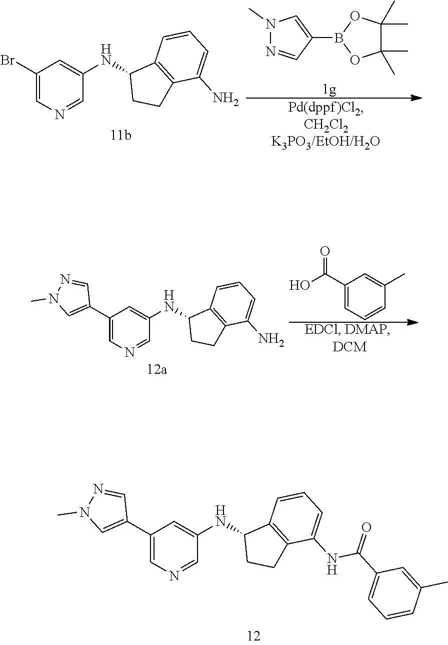

Step 1: To a solution of 11b (234 mg, 0.77 mmol), 1g (239 mg, 1.15 mmol) and K₃PO₄ (490 mg, 2.31 mmol) in a mixture of solvent ethanol (3 mL) and water (0.6 mL) was added Pd(dppf)Cl₂·CH₂Cl₂ (31 mg, 0.039 mmol). The resulting mixture was degassed with N₂ for three times, then heated to reflux and stirred until TLC showed the reaction was complete. After natural cooling to room temperature, the separated organic layer was concentrated to dryness and purified by column chromatography to give compound 12a (146 mg, yield: 62.0%). ESI-MS (m/z): 305.2 [M+H]⁺.

Step 2: To a solution of 12a (61 mg, 0.2 mmol), 3-methylbenzoic acid (34 mg, 0.3 mmol), and DMAP (1 mg, 0.01 mmol) in CH₂Cl₂ (0.5 mL) was added EDCI (58 mg, 0.3 mmol). The reaction mixture was stirred at room temperature until TLC showed the reaction was complete. The resulting mixture was concentrated to dryness in vacuum and purified by column chromatography to give the desired product 12 (35 mg, yield: 42.0%). ESI-MS (m/z): 424.2 [M+H]⁺. ¹H NMR (62 MHz, acetone-d₆) ¿ 9.09 (s, 1H), 8.13-8.01 (m, 3H), 8.78-8.74 (m, 3H), 7.68-7.59 (m, 1H), 7.44-7.17 (m, 5H), 5.62-5.20 (m, 2H), 3.92 (s, 3H), 3.01-2.85 (m, 2H), 2.65-2.44 (m, 1H), 2.41 (s, 3H), 1.90-1.62 (m, 1H).

### Example 13

### (S)-1-((5-(1-methyl-1H-pyrazol-4-yl) pyridin-3-yl)amino)-N-(m-tolyl)-2,3-dihydro-1H-indene-4-carboxamide

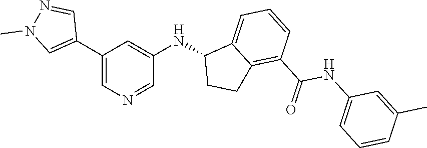

Compound 13 was prepared by the following steps:

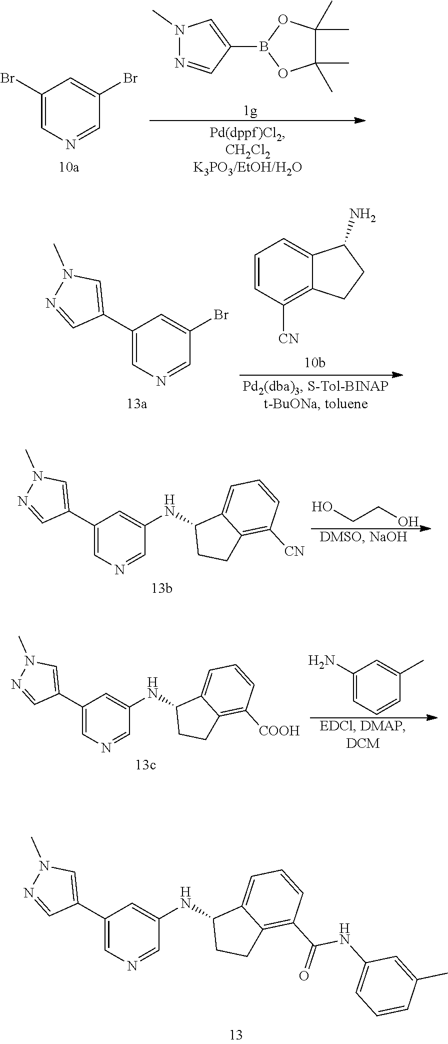

Step 1: To a solution of 3,5-dibromopyridine 10a (2370 mg, 10 mmol) in ethanol (12 mL) was added Pd(dppf)Cl₂. CH₂Cl₂ (82 mg, 0.1 mmol), 1g (2195 mg, 10.5 mmol), water (2.5 mL) and K₃PO₄ (6360 mg, 30 mmol). The resulting mixture was degassed with N₂ for three times, then heated to reflux and stirred for 2 h. TLC showed the reaction was complete. After natural cooling to room temperature, the separated organic layer was concentrated in vacuum and purified by column chromatography to give the desired product 13a (1888 mg, yield: 80.0%). ESI-MS (M/S): 238.1 [M+H]⁺.

Step 2: To a solution of 13a (947.2 mg, 4.1 mmol) in toluene (4 mL) was added (1S)-1-amino-2,3-dihydro-1H-indene-4-carbonitrile 10b (530 mg, 3.4 mmol), Pd₂(dba)₃ (153.6 mg, 0.05 mmol), S-Tol-BINAP (456 mg, 0.2 mmol) and sodium tert-butoxide (646.4 mg, 2.0 mmol). The resulting mixture was degassed with N₂ for three times, then heated to 80° C. and stirred for 6 h. TLC showed the reaction was complete. The mixture was concentrated in vacuum and purified by column chromatography to give the desired product 13b (711 mg, yield: 55.0%). ESI-MS (M/S): 316.3 [M+H]⁺.

Step 3: To a solution of 13b (158 mg, 0.5 mmol) in DMSO (1.8 mL) was added 50% aqueous NaOH (0.9 mL) and glycol (0.9 mL). The reaction mixture was refluxed overnight. TLC showed the reaction was complete. The reaction mixture was diluted with water, and then adjusted to pH=4 with concentrated hydrochloric acid. The large amount of solid precipitated was collected by filtration to give the desired product 13c (105 mg, yield: 63%). ESI-MS (M/S): 335.3 [M+H]⁺.

Step 4: To a solution of 13c (100 mg, 0.3 mmol) in CH₂Cl₂ (0.5 mL) was added 3-methyl aniline (64 mg, 0.6 mmol), DMAP (7 mg, 0.06 mmol), triethylamine (40 mg, 0.4 mmol) and EDCI (115 mg, 0.6 mmol). The reaction mixture was stirred at room temperature for 8 h. TLC showed the reaction was complete. The mixture was washed with water twice. The separated organic layer was dried, concentrated and purified by column chromatography to give the desired product 13 (98 mg, yield: 77.0%). ESI-MS (M/S): 424.1 [M+H]⁺. ¹H NMR (62 MHz, acetone-d₆) δ 10.10 (s, 1H), 8.18-7.81 (m, 4H), 7.64-6.80 (m, 8H), 6.35-6.03 (m, 1H), 5.33-4.93 (m, 1H), 3.87 (s, 3H), 3.17-2.60 (m, 3H), 2.31 (s, 3H), 2.07-1.55 (m, 1H).

### Example 14

### (S)—N-(5-chloropyridin-3-yl)-1-((5-(1-methyl-1H-pyrazol-4-yl) pyridin-3-yl)amino)-2,3-dihydro-1H-indene-4-carboxamide

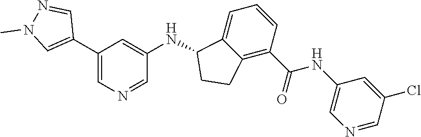 [SMILES: Cn1cc(-c2cncc(N[C@H]3CCc4c(C(=O)Nc5cncc(Cl)c5)cccc43)c2)cn1]

Compound 14aa was prepared by the following steps:

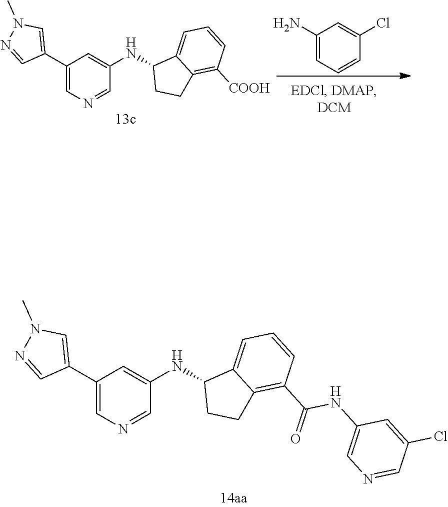 [SMILES: Cn1cc(-c2cncc(N[C@H]3CCc4c(C(=O)O)cccc43)c2)cn1 ; Nc1cccc(Cl)c1 ; Cn1cc(-c2cncc(N[C@H]3CCc4c(C(=O)Nc5cncc(Cl)c5)cccc43)c2)cn1]

To a solution of 13c (100 mg, 0.3 mmol) in CH₂Cl₂ (0.5 mL) was added 3-amino-5-chloropyridine (77.1 mg, 0.6 mmol), DMAP (7.3 mg, 0.06 mmol), triethylamine (40.4 mg, 0.4 mmol) and EDCI (115.2 mg, 0.6 mmol). The reaction mixture was stirred at room temperature for 8 h. TLC showed the reaction was complete. The mixture was washed with water twice. The separated organic layer was dried, concentrated and purified by column chromatography to give the desired product 14aa (91 mg, yield: 68%).

The preparation of compounds 14ab-14ba refers to the synthesis of compound 13 in example 13 or compound 14aa in example 14.

TABLE 4

| | | MS | |
|---|---|---|---|
| | | [M + | |
| Cpds | Structure | 1]⁺ | ¹H NMR |
| 14aa | 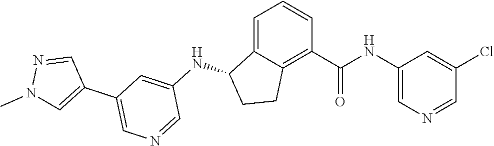 [SMILES: Cn1cc(-c2cncc(N[C@H]3CCc4c(C(=O)Nc5cncc(Cl)c5)cccc43)c2)cn1] | 445.2 | ¹H NMR (62 MHz, acetone-d₆) δ 10.63 (s, 1H), 8.84 (d, J = 1.6 Hz, 1H), 8.37 (s, 2H), 8.16-7.76 (m, 4H), 7.44 (t, J = 11.2 Hz, 4H), 6.24 (d, J = 8.4 Hz, 1H), 5.32- 4.95 (m, 1H), 3.86 (s, 3H), 3.21-2.56 (m, 3H), 2.00-1.64 (m, 1H). |
| 14ab | 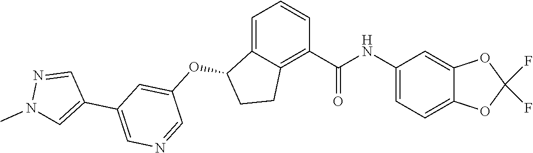 [SMILES: Cn1cc(-c2cncc(O[C@H]3CCc4c(C(=O)Nc5ccc6c(c5)OC(F)(F)O6)cccc43)c2)cn1] | 490.2 | ¹H NMR (400 MHz, DMSO-d₆) δ 10.46 (s, 1H), 8.17 (s, 1H), 8.05 (s, 1H), 7.96- 7.91 (m, 2H), 7.87 (s, 1H), 7.63 (d, J = 7.6 Hz, 1H), 7.49 (t, J = 8.3 Hz, 2H), 7.38 (dd, J = 17.3, 8.6 Hz, 2H), 7.27 (s, 1H), 6.26 (d, J = 8.5 Hz, 1H), 5.15 (q, J = 7.7 Hz, 1H), 3.27-3.19 (m, 1H), 3.04 (dd, J = 16.7, 8.3 Hz, 1H), 1.88-1.79 (m, 1H). |
| 14ac | 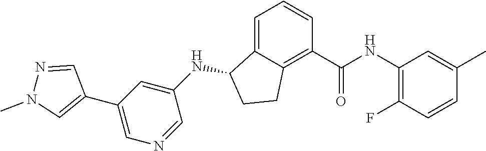 | 442.2 | ¹H NMR (62 MHz, acetone-d₆) δ 9.92 (s, 1H), 8.20-6.95 (m, 11H), 6.24 (d, J = 8.4 Hz, 1H), 5.39-4.93 (m, 1H), 3.89 (s, 3H), 3.27-2.62 (m, 3H), 2.33 (s, 3H), 2.03-1.65 (m, 1H). |
| 14ad | 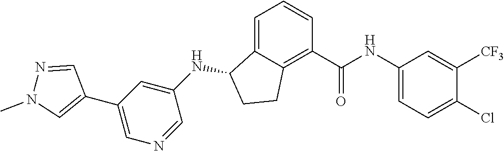 [SMILES: Cn1cc(-c2cncc(N[C@H]3CCc4c(C(=O)Nc5ccc(Cl)c(C(F)(F)F)c5)cccc43)c2)cn1] | 512.1 | ¹H NMR (62 MHz, acetone-d₆) δ 10.64 (s, 1H), 8.37 (d, J = 2.3 Hz, 1H), 8.15 (s, 1H), 7.93 (dt, J = 9.6, 3.7 Hz, 5H), 7.58 (d, J = 4.0 Hz, 1H), 7.52-7.18 (m, 3H), 6.24 (d, J = 8.5 Hz, 1H), 5.39-4.91 (m, 1H), 3.87 (s, 3H), 3.25-2.58 (m, 3H), 2.07-1.59 (m, 1H). |
| 14ae | 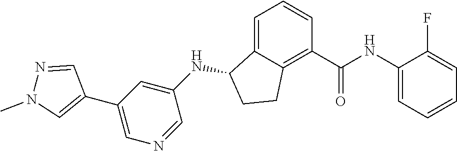 [SMILES: Cn1cc(-c2cncc(N[C@H]3CCc4c(C(=O)Nc5ccccc5F)cccc43)c2)cn1] | 428.2 | ¹H NMR (62 MHz, acetone-d₆) δ 9.98 (s, 1H), 8.18-7.84 (m, 4H), 7.76-7.51 (m, 2H), 7.46-7.09 (m, 6H), 6.23 (d, J = 8.4 Hz, 1H), 5.34-4.95 (m, 1H), 3.87 (s, 3H), 3.18- 2.50 (m, 3H), 2.06-1.62 (m, 1H). |
| 14af | 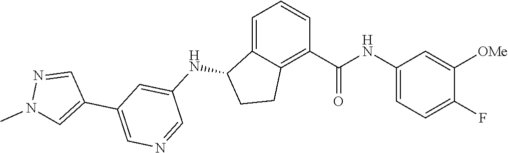 [SMILES: COc1cc(NC(=O)c2cccc3c2CC[C@@H]3Nc2cncc(-c3cnn(C)c3)c2)ccc1F] | 458.2 | ¹H NMR (62 MHz, acetone-d₆) δ 10.23 (s, 1H), 8.16-7.07 (m, 11H), 6.22 (d, J = 8.4 Hz, 1H), 5.31-4.93 (m, 1H), 3.86 (s, 3H), 3.83 (s, 3H), 3.25-2.59 (m, 3H), 1.99- 1.53 (m, 1H). |
| 14ag | 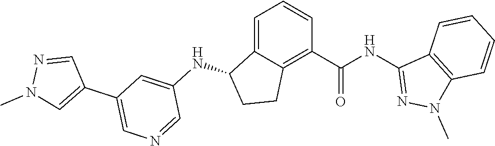 [SMILES: Cn1cc(-c2cncc(N[C@H]3CCc4c(C(=O)Nc5nn(C)c6ccccc56)cccc43)c2)cn1] | 464.2 | ¹H NMR (62 MHz, acetone-d₆) δ 9.90 (s, 1H), 8.44-6.73 (m, 12H), 5.54-4.83 (m, 1H), 3.93 (d, J = 5.1 Hz, 6H), 3.36 (dt, J = 10.2, 6.9 Hz, 2H), 2.90-2.17 (m, 2H), 1.74 (t, J = 8.3 Hz, 1H). |
| 14ah | 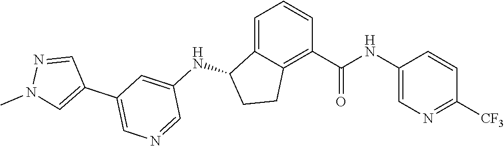 [SMILES: Cn1cc(-c2cncc(N[C@H]3CCc4c(C(=O)Nc5ccc(C(F)(F)F)nc5)cccc43)c2)cn1] | 479.2 | ¹H NMR (62 MHz, acetone-d₆) δ 10.81 (s, 1H), 9.06 (s, 1H), 8.76-6.82 (m, 10H), 6.25 (d, J = 8.3 Hz, 1H), 5.10 (dq, J = 10.8, 6.2, 5.2 Hz, 1H), 3.87 (s, 3H), 3.19-2.64 (m, 2H), 2.45-1.49 (m, 2H). |
| 14ai | 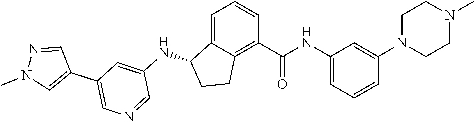 | 508.3 | ¹H NMR (62 MHz, acetone-d₆) δ 9.90 (s, 1H), 8.13-7.55 (m, 5H), 7.42-6.88 (m, 5H), 6.62 (d, J = 7.0 Hz, 2H), 6.07 (d, J = 8.7 Hz, 1H), 5.23-4.67 (m, 1H), 3.68 (s, 3H), 3.15-2.88 (m, 7H), 2.41 (d, J = 5.1 Hz, 4H), 2.16 (s, 3H), 1.83-1.56 (m, 1H). |
| 14aj | 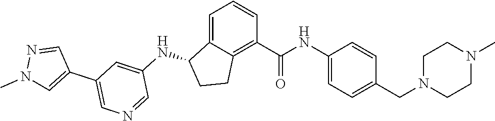 | 522.3 | ¹H NMR (400 MHz, DMSO-d₆) δ 10.21 (s, 1H), 8.17 (s, 1H), 8.05 (s, 1H), 7.91 (d, J = 28.7 Hz, 2H), 7.70 (d, J = 8.1 Hz, 2H), 7.61 (d, J = 7.6 Hz, 1H), 7.45 (d, J = 7.5 Hz, 1H), 7.35 (t, J = 7.7 Hz, 1H), 7.25 (d, J = 8.1 Hz, 3H), 6.25 (d, J = 8.5 Hz, 1H), 5.14 (d, J = 7.9 Hz, 1H), 3.87 (s, 3H), 3.41 (s, 3H), 3.23 (dd, J = 16.7, 9.6 Hz, 1H), |
| | | | 3.04 (dd, J = 16.7, 8.5 Hz, 1H), 2.58 (dd, |
| | | | J = 13.0, 5.6 Hz, 2H), 2.41-2.27 (m, 6H), |
| | | | 2.15 (s, 3H), 1.86-1.79 (m, 1H). |
| 14ak | 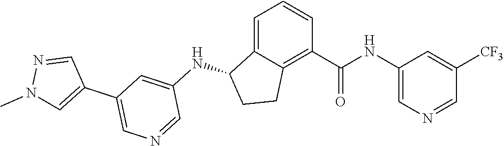 [SMILES: Cn1cc(-c2cncc(N[C@H]3CCc4c(C(=O)Nc5cncc(C(F)(F)F)c5)cccc43)c2)cn1] | 479.2 | ¹H NMR (62 MHz, acetone-d₆) δ 9.94 (s, 1H), 9.17 (d, 1H), 8.85-8.57 (m, 2H), 8.19- 8.08 (m, 1H), 8.07-7.90 (m, 2H), 7.89-7.57 (m, 2H), 7.56-7.18 (m, 3H), 5.64-4.94 (m, 2H), 3.92 (s, 3H), 3.53-2.97 (m, 2H), 2.72- 2.21 (m, 1H), 1.84-1.10 (m, 1H). |
| 14al | 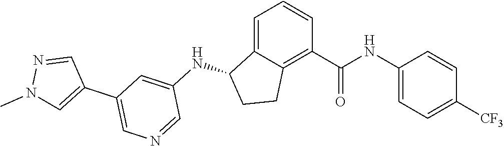 [SMILES: Cn1cc(-c2cncc(N[C@H]3CCc4c(C(=O)Nc5ccc(C(F)(F)F)cc5)cccc43)c2)cn1] | 478.2 | ¹H NMR (62 MHz, acetone-d₆) δ 9.78 (s, 1H), 8.29-7.12 (m, 12H), 5.60-4.93 (m, 2H), 3.91 (s, 3H), 3.55-3.04 (m, 2H), 2.46 (td, J = 14.2, 13.3, 5.7 Hz, 1H), 1.92-1.55 (m, 1H). |
| 14am | 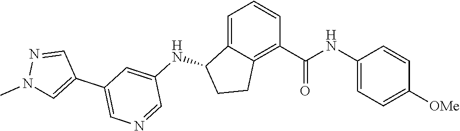 [SMILES: COc1ccc(NC(=O)c2cccc3c2CC[C@@H]3Nc2cncc(-c3cnn(C)c3)c2)cc1] | 440.2 | ¹H NMR (62 MHz, acetone-d₆) δ 10.08 (s, 1H), 8.48-6.75 (m, 12H), 6.23 (d, J = 8.4 Hz, 1H), 5.40-4.88 (m, 1H), 3.87 (s, 3H), 3.74 (s, 3H), 3.19-2.62 (m, 3H), 2.20-1.57 (m, 1H). |
| 14an | 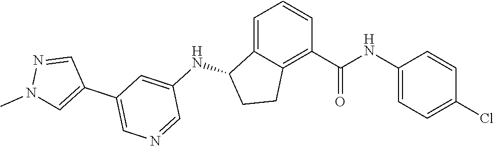 [SMILES: Cn1cc(-c2cncc(N[C@H]3CCc4c(C(=O)Nc5ccc(Cl)cc5)cccc43)c2)cn1] | 444.2 | ¹H NMR (62 MHz, acetone-d₆) δ 10.35 (s, 1H), 8.34-7.13 (m, 12H), 6.22 (d, J = 8.3 Hz, 1H), 5.11 (dt, J = 11.9, 5.8 Hz, 1H), 3.86 (s, 3H), 3.20-2.62 (m, 3H), 2.18-1.55 (m, 1H). |
| 14ao | 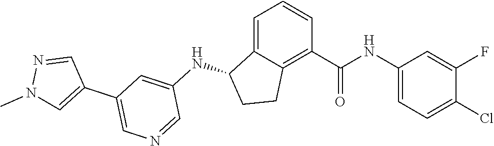 [SMILES: Cn1cc(-c2cncc(N[C@H]3CCc4c(C(=O)Nc5ccc(Cl)c(F)c5)cccc43)c2)cn1] | 462.1 | ¹H NMR (62 MHz, acetone-d₆) δ 10.51 (s, 1H), 8.30-7.07 (m, 11H), 6.20 (d, J = 8.4 Hz, 1H), 5.40-4.86 (m, 1H), 3.83 (s, 3H), 3.21-2.66 (m, 3H), 2.07-1.43 (m, 1H). |
| 14ap | 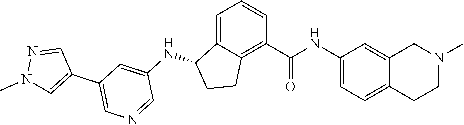 | 479.3 | ¹H NMR (400 MHz, DMSO-d₆) δ 10.11 (s, 1H), 8.17 (s, 1H), 8.05 (d, J = 1.7 Hz, 1H), 7.94 (d, J = 2.6 Hz, 1H), 7.87 (s, 1H), 7.60 (d, J = 7.5 Hz, 1H), 7.55-7.42 (m, 3H), 7.34 (t, J = 7.6 Hz, 1H), 7.26 (s, 1H), 7.07 (d, J = 8.2 Hz, 1H), 6.25 (d, J = 8.5 Hz, 1H), 5.13 (q, J = 7.6 Hz, 1H), 3.87 (s, 3H), 3.47 (s, 2H), 3.26-3.14 (m, 1H), 2.78 (s, 2H), 2.59 (s, 3H), 2.34 (s, |
| | | | 3H), 1.82 (dd, J = 12.3, 7.9 Hz, 1H). |
| 14aq | 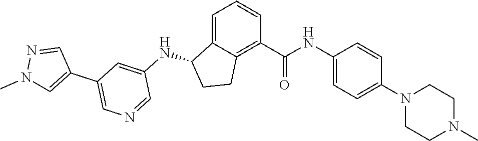 | 508.3 | ¹H NMR (400 MHz, DMSO-d₆) δ 10.01 (s, 1H), 8.17 (s, 1H), 8.05 (d, 1H), 7.94 (d, 1H), 7.87 (d, 1H), 7.68-7.53 (m, 3H), 7.43 (d, 1H), 7.33 (t, 1H), 7.26 (d, 1H), 6.93 (d, 2H), 6.24 (d, 1H), 5.13 (q, 1H), 3.87 (s, 3H), 3.33-3.17 (m, 2H), 3.16-2.95 (m, 5H), 2.65-2.56 (m, 1H), 2.44 (s, 3H), 2.22 (s, 3H), 1.81 (q, 1H). |
| 14ar | 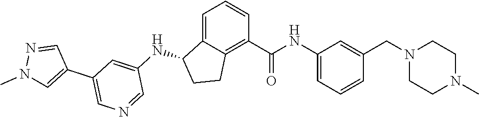 | 522.3 | ¹H NMR (400 MHz, DMSO-d₆) δ 10.21 (s, 1H), 8.17 (s, 1H), 8.05 (d, J = 1.8 Hz, 1H), 7.94 (d, J = 2.6 Hz, 1H), 7.87 (s, 1H), 7.77 (s, 1H), 7.66 (d, J = 7.5 Hz, 1H), 7.62 (d, J = 7.5 Hz, 1H), 7.45 (d, J = 7.5 Hz, 1H), 7.35 (t, J = 7.5 Hz, 1H), 7.31-7.18 (m, 3H), 7.03 (d, J = 7.5 Hz, 1H), 6.25 (d, J = 8.5 Hz, 1H), 5.14 (q, J = 7.6 Hz, 1H), 3.87 (s, |
| | | | 3H), 3.44 (s, 2H), 3.30-3.17 (m, 1H), 3.10- |
| | | | 2.99 (m, 1H), 2.78-2.53 (m, 1H), 2.46-2.28 |
| | | | (m, 7H), 2.15 (s, 3H), 1.91-1.67 (m, 1H). |
| 14as | 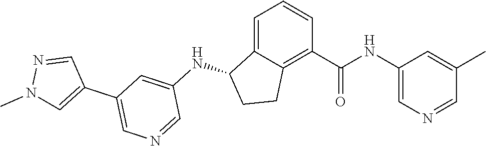 | 425.2 | ¹H NMR (400 MHz, DMSO-d₆) δ 10.38 (s, 1H), 8.70 (d, J = 2.3 Hz, 1H), 8.17 (s, 2H), 8.10-8.03 (m, 2H), 7.94 (d, J = 2.6 Hz, 1H), 7.87 (s, 1H), 7.66 (d, J = 7.5 Hz, 1H), 7.48 (d, J = 7.4 Hz, 1H), 7.38 (t, J = 7.6 Hz, 1H), 7.27 (t, J = 2.2 Hz, 1H), 6.26 (d, J = 8.4 Hz, 1H), 5.15 (d, J = 7.8 Hz, 1H), 3.87 (s, 3H), 3.32-3.02 (m, 2H), 2.74-2.53 (m, 2H), 1.83 (s, 3H). |
| 14at | 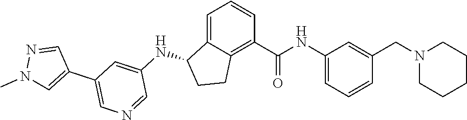 [SMILES: Cn1cc(-c2cncc(N[C@H]3CCc4c(C(=O)Nc5cccc(CN6CCCCC6)c5)cccc43)c2)cn1] | 507.3 | ¹H NMR (400 MHz, DMSO-d₆) δ 10.21 (s, 1H), 8.17 (s, 1H), 8.05 (s 1H), 7.95 (s, 1H), 7.87 (s, 1H), 7.72 (s, 1H), 7.67-7.61 (m, 2H), 7.45 (d, J = 7.4 Hz, 1H), 7.34 (t, J = 7.6 Hz, 1H), 7.31-7.21 (m, 2H), 7.01 (d, J = 7.5 Hz, 1H), 6.25 (d, J = 8.4 Hz, 1H), 5.17-5.11 (m, 1H), 3.87 (s, 3H), 3.28-3.21 (m, 1H), 3.09-3.01 (m, 1H), 2.62-2.54 (m, |
| | | | 1H), 2.39-2.26 (m, 4H), 1.87-1.80 (m, |
| | | | 1H), 1.53-1.47 (m, 4H), 1.41-1.37 (m, 2H). |
| 14au | 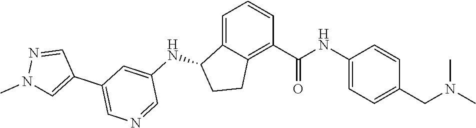 | 467.3 | ¹H NMR (400 MHz, DMSO-d₆) δ 10.21 (s, 1H), 8.17 (s, 1H), 8.05 (s, 1H), 7.94 (s, 1H), 7.87 (s, 1H), 7.71 (d, J = 8.1 Hz, 2H), 7.61 (d, J = 7.5 Hz, 1H), 7.45 (d, J = 7.4 Hz, 1H), 7.35 (t, J = 7.5 Hz, 1H), 7.26-7.24 (m, 3H), 6.25 (d, J = 8.4 Hz, 1H), 5.17- 5.11 (m, 1H), 3.87 (s, 3H), 3.36 (s, 2H), 3.27-3.20 (m 1H), 3.09-3.01 (m, 1H), 2.61- 2.56 (m, 1H), 2.15 (s, 6H), 1.87-1.78 (m, |
| | | | 1H). |
| 14av | 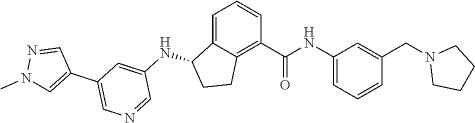 [SMILES: Cn1cc(-c2cncc(N[C@H]3CCc4c(C(=O)Nc5cccc(CN6CCCC6)c5)cccc43)c2)cn1] | 493.3 | ¹H NMR (400 MHz, DMSO-d₆) δ 10.21 (s, 1H), 8.17 (s, 1H), 8.05 (s, 1H), 7.95 (s, 1H), 7.88 (s, 1H), 7.76 (s, 1H), 7.66-7.62 (m, 2H), 7.45 (d, J = 7.5 Hz, 1H), 7.36- 7.26 (m, 3H), 7.03 (d, J = 7.5 Hz, 1H), 6.26 (d, J = 8.4 Hz, 1H), 5.17-5.11 (m, 1H), 3.87 (s, 3H), 3.57 (s, 2H), 3.28-3.21 (m, 1H), 3.10-3.01 (m, 1H), 2.60-2.57 (m, |
| | | | 1H), 2.45 (s, 4H), 1.85-1.80 (m, 1H), |
| | | | 1.71 (s, 4H). |
| 14aw | 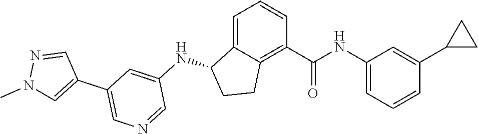 [SMILES: Cn1cc(-c2cncc(N[C@H]3CCc4c(C(=O)Nc5cccc(C6CC6)c5)cccc43)c2)cn1] | 450.2 | ¹H NMR (400 MHz, DMSO-d6) δ 10.15 (s, 1H), 8.17 (s, 1H), 8.11-8.03 (m, 1H), 7.95 (d, J = 2.5 Hz, 1H), 7.88 (s, 1H), 7.63-7.43 (m, 4H), 7.42-7.15 (m, 3H), 6.83 (d, J = 7.7 Hz, 1H), 6.26 (d, J = 8.4 Hz, 1H), 5.14 (q, J = 7.6 Hz, 1H), 3.87 (s, 3H), 3.31-3.20 (m, 1H), 3.05 (dt, J = 16.6, 8.1 Hz, 1H), 2.59 (ddd, J = 11.8, 7.7, 3.7 Hz, 1H), 1.95-1.78 (m, 2H), 0.96 |
| | | | (dt, J = 8.5, 3.2 Hz, 2H), 0.72-0.60 (m, |
| | | | 2H). |
| 14ax | 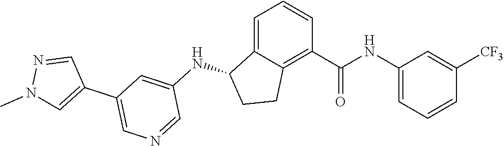 [SMILES: Cn1cc(-c2cncc(N[C@H]3CCc4c(C(=O)Nc5cccc(C(F)(F)F)c5)cccc43)c2)cn1] | 478.2 | ¹H NMR (400 MHz, DMSO-d6) δ 10.57 (s, 1H), 8.27 (d, J = 2.0 Hz, 1H), 8.17 (s, 1H), 8.07-7.87 (m, 4H), 7.71-7.57 (m, 2H), 7.42 (dt, J = 37.2, 7.8 Hz, 3H), 7.27 (t, J = 2.3 Hz, 1H), 6.27 (d, J = 8.4 Hz, 1H), 5.15 (q, J = 7.6 Hz, 1H), 3.87 (s, 3H), 3.26 (ddd, J = 16.8, 8.6, 3.6 Hz, 1H), 3.06 (dt, J = 16.7, 8.2 Hz, 1H), 2.60 (ddd, J = 11.8, 7.8, 3.8 Hz, 1H), 1.88-1.80 (m, 1H). |
| 14ay | 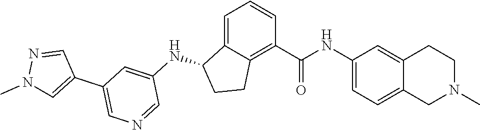 | 479.3 | ¹H NMR (400 MHz, DMSO-d6) δ 10.11 (s, 1H), 8.17 (s, 1H), 8.05 (s, 1H), 7.94 (s, 1H), 7.87 (s, 1H), 7.61-7.58 (m, 2H), 7.46- 7.42 (m, 2H), 7.36-7.32 m, 1H), 7.26 (s, 1H), 7.02 (d, J = 8.3 Hz, 1H), 6.24 (d, J = 8.4 Hz, 1H), 5.17-5.11 (m, 1H), 3.87 (s, 3H), 3.48 (s, 2H), 3.26-3.18 (m, 2H), 3.11- 2.98 (m, 1H), 2.83 (s, 2H), 2.63 (s, 2H), 2.37 (s, 3H), 1.82 (s, 1H). |
| 14az | 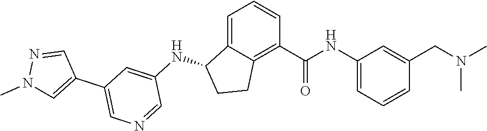 | 467.3 | ¹H NMR (400 MHz, DMSO-d6) δ 10.20 (s, 1H), 8.17 (s, 1H), 8.05 (m, 1H), 7.95 (m, 1H), 7.87 (s, 1H), 7.77 (s, 1H), 7.66- 7.62 (m, 2H), 7.45 (d, J = 7.4 Hz, 1H), 7.41-7.21 (m, 3H), 7.02 (d, J = 7.5 Hz, 1H), 6.25 (d, J = 8.4 Hz, 1H), 5.17-5.11 (m, 1H), 3.87 (s, 3H), 3.38 (s, 2H), 3.28- 3.21 (m, 1H), 3.10-3.01 (m, 1H), 2.62- 2.54 (m, 1H), 2.17 (s, 6H), 1.87-1.78 (m, |
| | | | 1H). |
| 14ba | 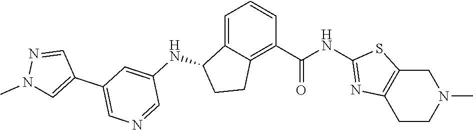 | 486.2 | ¹H NMR (400 MHz, DMSO-d₆) δ 12.40 (s, 1H), 8.17 (s, 1H), 8.05 (s, 1H), 7.93 (s, 1H), 7.87 (s, 1H), 7.76 (d, J = 7.6 Hz, 1H), 7.49 (d, J = 7.5 Hz, 1H), 7.35 (t, J = 7.6 Hz, 1H), 7.27-7.25 (m, 1H), 6.26 (d, J = 8.5 Hz, 1H), 5.17-5.11 (m, 1H), 3.87 (s, 3H), 3.54 (s, 2H), 3.30-3.23 (m, 1H), 3.13-3.05 (m, 1H), 2.75-2.68 (m, 4H), 2.61-2.53 (m, 1H), 2.40 (s, 3H), 1.87-1.80 |
| | | | (m, 1H). |

### Example 15

### (S)-3-methyl-N-(1-((5-(1-methyl-1H-pyrazol-4-yl) pyridin-3-yl)oxy)-2,3-dihydro-1H-inden-4-yl)benzamide

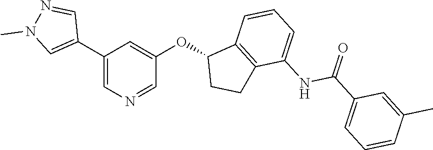

Compound 15 was prepared by the following steps.

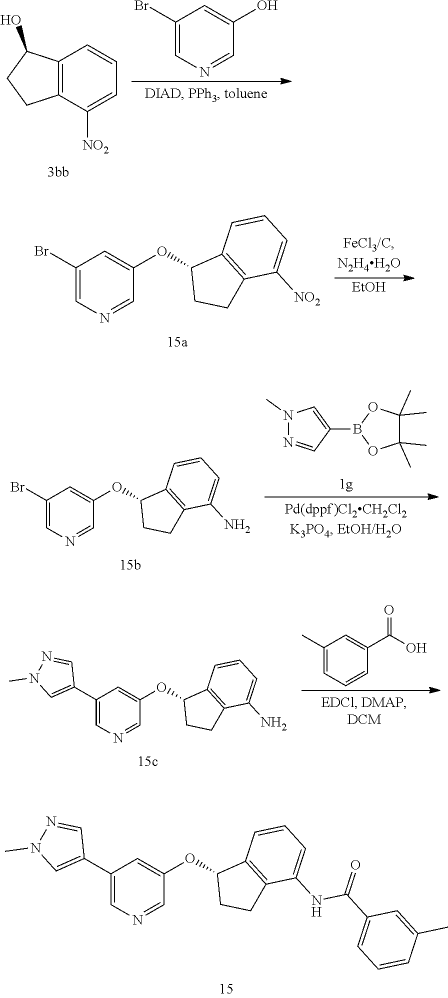

Step 1: To a stirred solution of 3bb (537 mg, 3.0 mmol), 5-bromo-3-hydroxypyridine (870 mg, 5.0 mmol) and PPh₃ (1.31 g, 5.0 mmol) in toluene (10 mL) cooled to −20° C. was added dropwise DIAD (1.10 g, 5.0 mmol) while keeping inner temperature below −10° C. under N₂. The resulting mixture was kept at −10° C. until TLC showed the reaction was complete. Then water (0.5 mL) was added to the reaction mixture, and the resulting mixture was stirred for another 1 h at 0° C. The solid precipitated was filtered and washed with toluene. The combined filtrates were washed with 1N aqueous NaOH (20 mL), dried over Na₂SO₄, and concentrated to dryness in vacuum. The residue was purified by column chromatography to give the desired product 15a (651 mg, yield: 65.0%). ESI-MS (m/z): 335.1 [M+H]⁺.

Step 2: To a stirred mixture of 15a (500 mg, 1.5 mmol), FeCl₃ (24 mg, 0.15 mmol) and active carbon (50 mg) in ethanol (10 mL) heated to reflux was added dropwise 80% hydrazine hydrate (470 mg, 7.5 mmol). The resulting mixture was refluxed until TLC showed the reaction was complete. After natural cooling to room temperature, the reaction mixture was concentrated to dryness in vacuum. The residue was redissolved in ethyl acetate (20 mL), and washed with water (20 mL*2). The organic layer was dried over Na₂SO₄, concentrated to dryness in vacuum and purified by column chromatography to give the desired product 15b (402 mg, yield: 88%). ESI-MS (m/z): 305.1 [M+H]⁺.

Step 3: To a solution of 3,5-dibromopyridine 15b (305 mg, 1.0 mmol), 1g (312 mg, 1.5 mmol). K₃PO₄ (636 mg, 3.0 mmol) in a mixture of solvent ethanol (5 mL) and water (1 mL) was added Pd(dppf)Cl₂·CH₂Cl₂ (40 mg, 0.05 mmol). The resulting mixture was degassed with N₂ for three times, then heated to reflux and stirred until TLC showed the reaction was complete. After natural cooling to room temperature, the separated organic layer was concentrated to dryness in vacuum and purified by column chromatography to give the desired product 15c (208 mg, yield: 68.0%). ESI-MS (m/z): 307.2 [M+H]⁺.

Step 4: To a solution of 15c (91 mg, 0.3 mmol), 3-methylbenzoic acid (68 mg, 0.5 mmol), DMAP (4 mg, 0.03 mmol) in CH₂Cl₂ (0.5 mL) was added EDCI (96 mg, 0.5 mmol). The reaction mixture was stirred at room temperature until TLC showed the reaction was complete, then concentrated to dryness in vacuum and purified by column chromatography to give 15 (72 mg, yield: 58.0%). ESI-MS (m/z): 425.1 [M+H]⁺. ¹H NMR (62 MHz, acetone-d₆) δ 9.99 (s, 1H), 8.46 (s, 1H), 8.29 (s, 1H), 8.19 (d, J=2.7 Hz, 1H), 8.01 (s, 1H), 7.79-7.70 (m, 3H), 7.45-7.23 (m, 5H), 6.15-6.06 (m, 1H), 3.88 (s, 3H), 3.13-2.76 (m, 2H), 2.70-2.60 (m, 1H), 2.40 (s, 3H), 2.07 (s, 1H).

### Example 16

### (R)-3-methyl-N-(1-((5-(1-methyl-1H-pyrazol-4-yl) pyridin-3-yl)oxy)-2,3-dihydro-1H-inden-4-yl)benzamide

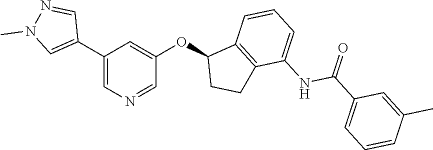

The preparation of compound 16 refers to the synthesis of compound 15 in example 15.

ESI-MS (m/z): 425.1 [M+H]⁺. ¹H NMR (62 MHz, acetone-d₆) δ 9.99 (s, 1H), 8.45 (s, 1H), 8.29 (s, 1H), 8.19 (s, 1H), 8.01 (s, 1H), 7.79-7.70 (m, 3H), 7.44-7.22 (m, 5H), 6.12-6.05 (m, 1H), 3.88 (s, 3H), 3.13-2.76 (m, 2H), 2.70-2.60 (m, 1H), 2.40 (s, 3H), 2.08 (s, 1H).

### Example 17

### (S)-1-((5-(1-methyl-1H-pyrazol-4-yl) pyridin-3-yl)oxy)-N-(4-(trifluoromethyl)phenyl)-2,3-dihydro-1H-indene-4-carboxamide

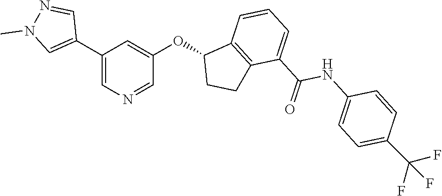 [SMILES: Cn1cc(-c2cncc(O[C@H]3CCc4c(C(=O)Nc5ccc(C(F)(F)F)cc5)cccc43)c2)cn1]

Compound 17 was prepared by the following steps:

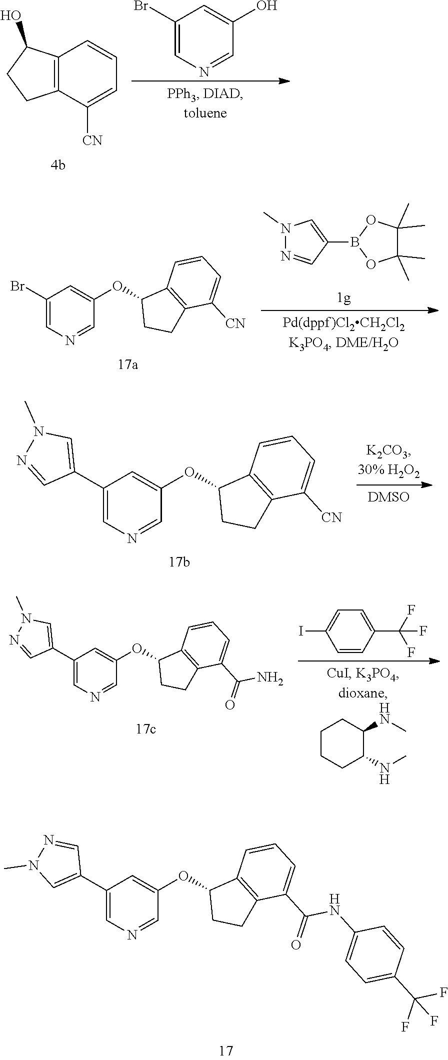

Step 1: To a solution of 4b (1.9 g, 11.9 mmol), 5-bromo-3-hydroxypyridine (3.1 g, 17.9 mmol) and PPh₃ (5.0 g, 17.9 mmol) in toluene (19 mL) was added dropwise DIAD (3.6 g, 17.9 mmol) while keeping the inner temperature below −10° C. under N₂. The resulting mixture was stirred at −10° C. for another 1 h. TLC showed the reaction was complete. Then water (1 mL) was added to the reaction mixture, and the resulting mixture was allowed to warm to room temperature and stirred for 0.5 h. The solid precipitated was filtered and washed with toluene. The combined filtrates were concentrated to dryness in vacuum, and purified by column chromatography to give the desired product 17a (1.9 g, yield: 43.6%). ESI-MS (m/z): 315.1 [M+H]⁺.

Step 2: To a mixture of 17a (1.9 g, 6.0 mmol), 1g (1.9 g, 9.0 mmol), KaPO₄ (3.8 g, 18.0 mmol) and Pd(dppf)Cl₂·CH₂Cl₂ (245 mg, 0.3 mmol) was added DME (8.4 mL) and water (3.2 mL). The resulting mixture was refluxed under N₂ for 1 h. TLC showed the reaction was complete. The reaction mixture was diluted with CH₂Cl₂ (200 mL), washed with water (50 mL*2), and dried over Na₂SO₄. After concentration, the mixture was purified by column chromatography to give product 17b (1.7 g, yield: 89.6%). ESI-MS (m/z): 317.1 [M+H]⁺.

Step 3: To a suspension of 17b (1235 mg, 3.9 mmol) and K₂CO₃ (1619 mg, 11.7 mmol) in DMSO (14.4 mL) was added dropwise 30% H₂O₂ (7.2 mL) slowly. The reaction mixture was stirred at room temperature for 1 h. After the reaction was complete, the reaction mixture was diluted with water (50 mL), and extracted with ethyl acetate (150 mL*4). The combined organic layers were washed with water (30 mL*2), dried over Na₂SO₄ and concentrated. The residue was purified by silica gel chromatography to give product 17c (412 mg, yield: 31.6%). ESI-MS (m/z): 335.1 [M+H]⁺.

Step 4: A mixture of 17c (100 mg, 0.3 mmol), 4-iodotrifluorotoluene (98 mg, 0.36 mmol). K₃PO₄ (127 mg, 0.6 mmol), CuI (6 mg, 0.03 mmol) and (1R,2R)-(−)-N,N′-dimethyl-1, 2-cyclohexanediamine (4.3 mg, 0.03 mmol) in 1,4-dioxane (1 mL) was degassed with N₂ for three times, then reached at 80° C. until TLC showed the reaction was complete. After cooling down, the reaction mixture was diluted with ethyl acetate (50 mL), washed with ammonium hydroxide (10 mL*2) and water (10 mL). The organic layer was dried over Na₂SO₄, concentrated to dryness in vacuum and purified by column chromatography to give product 17 (91 mg, yield: 63.3%). ESI-MS (m/z): 479.1 [M+H]⁺. ¹H NMR (400 MHz, DMSO-d₆) δ 10.64 (s, 1H), 8.47 (d, J=1.9 Hz, 1H), 8.31 (s, 1H), 8.19 (d, J=2.8 Hz, 1H), 8.05-7.96 (m, 3H), 7.79-7.70 (m, 4H), 7.65 (d, J=7.4 Hz, 1H), 7.45 (t, J=7.6 Hz, 1H), 6.08 (dd, J=6.6, 3.7 Hz, 1H), 3.89 (s, 3H), 3.34-3.25 (m, 1H), 3.20-3.08 (m, 1H), 2.69-2.59 (m, 1H), 2.18-2.05 (m, 1H).

### Example 18

### (S)—N-(2-methyl-1,2,3,4-tetrahydroisoquinolin-7-yl)-1-((5-(1-methyl-1H-pyrazol-4-yl) pyridin-3-yl)oxy)-2,3-dihydro-1H-indene-4-carboxamide

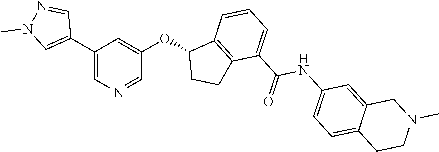

Compound 18 was prepared by the following steps:

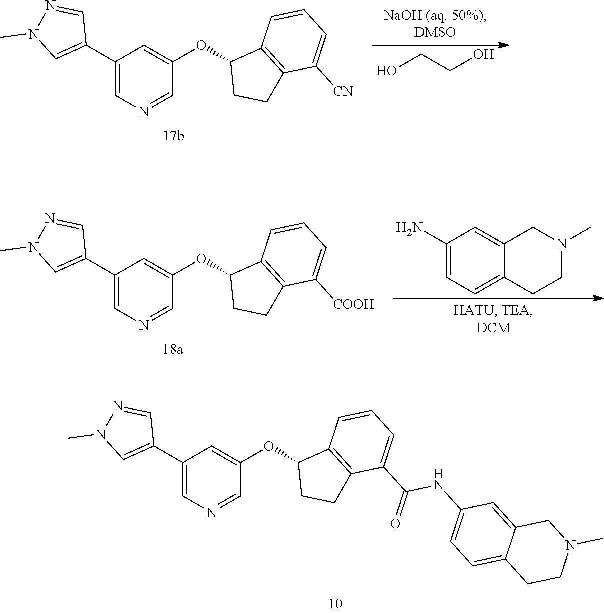

Step 1: To a stirred solution of 17b (948 mg, 3.0 mmol) in DMSO (5.0 mL) was added 50% aqueous NaOH (5.0 mL) and glycol (5.0 mL). The reaction mixture was heated to 110° C. and stirred until TLC showed the reaction was complete. After natural cooling to below 30° C., the mixture was adjusted to pH=6-7 with 1N hydrochloric acid. The large amount of solid precipitated was filtered and the filter cake was washed with water (5.0 mL) and dried in vacuum at 50° C. to give product 18a as a light brown solid (665 mg, yield: 66.5%).

Step 2: To a solution of 18a (67 mg, 0.2 mmol) and 2-methyl-1,2,3, 4-tetrahydroisoquinoline-7-amine (65 mg, 0.4 mmol) in CH₂Cl₂ (0.5 mL) was added triethylamine (40 mg, 0.4 mmol) and HATU (152 mg, 0.4 mmol). The reaction mixture was stirred at 25° C. until TLC showed the reaction was complete, then concentrated to dryness in vacuum and purified by column chromatography to give product 18 (18 mg, yield: 19.0%). ESI-MS (m/z): 480.2 [M+H]⁺. ¹H NMR (400 MHz, DMSO-d₆) δ 10.17 (s, 1H), 8.47 (d, J=1.6 Hz, 1H), 8.31 (s, 1H), 8.19 (d, J=2.7 Hz, 1H), 8.03 (s, 1H), 7.73-7.67 (m, 2H), 7.61 (d, J=7.5 Hz, 1H), 7.54-7.45 (m, 2H), 7.41 (t. J=7.6 Hz, 1H), 7.09 (d, J=8.3 Hz, 1H), 6.07 (dd, J=6.6, 3.7 Hz, 1H), 3.89 (s, 3H), 3.54 (s, 2H), 3.30-3.25 (m, 1H), 3.20-3.05 (m, 1H), 2.80 (1, J=5.8 Hz, 2H), 2.69-2.57 (m, 3H), 2.39 (s, 3H), 2.16-2.04 (m, 2H).

### Example 19

### (S)—N-(5-chloropyridin-3-yl)-1-((5-(1-methyl-1H-pyrazol-4-yl) pyridin-3-yl)oxy)-2,3-dihydro-1H-indene-4-carboxamide

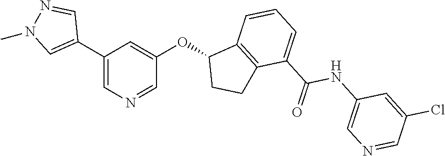 [SMILES: Cn1cc(-c2cncc(O[C@H]3CCc4c(C(=O)Nc5cncc(Cl)c5)cccc43)c2)cn1]

Compound 19a was prepared by the following steps:

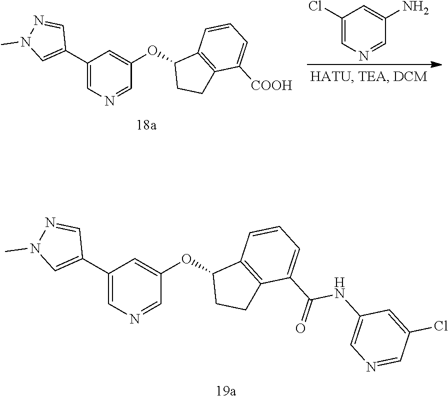 [SMILES: Cn1cc(-c2cncc(O[C@H]3CCc4c(C(=O)O)cccc43)c2)cn1 ; Nc1cncc(Cl)c1 ; Cn1cc(-c2cncc(O[C@H]3CCc4c(C(=O)Nc5cncc(Cl)c5)cccc43)c2)cn1]

To a solution of 18a (34 mg, 0.1 mmol) and 3-amino-5-chloropyridine (33 mg, 0.2 mmol) in CH₂Cl₂ (0.5 mL) was added triethylamine (20 mg, 0.2 mmol) and HATU (76 mg, 0.2 mmol). The reaction mixture was stirred at 25° C. until TLC showed the reaction was complete, then concentrated to dryness in vacuum and purified by column chromatography to give product 19a (18 mg, yield: 38.0%).

### (S)—N-(2,6-dimethylpyridin-4-yl)-1-((5-(1-methyl-1H-pyrazol-4-yl) pyridin-3-yl)oxy)-2,3-dihydro-1H-indene-4-carboxamide

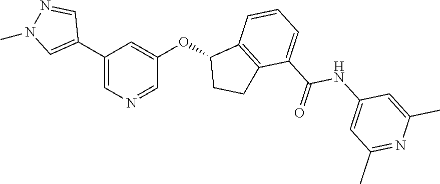

Compound 19k was prepared by the following steps:

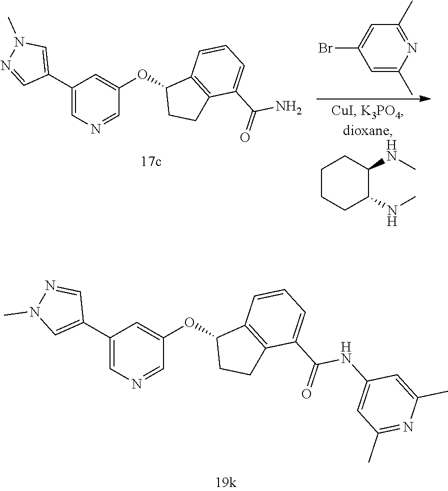

A mixture of 3d (50 mg, 0.12 mmol), 4-Bromo-2,6-dimethylpyridine (49 mg, 0.18 mmol), KaPO₄ (64 mg, 0.3 mmol), CuI (4 mg, 0.02 mmol) and (1R,2R)-(−)-N,N′-dimethyl-1, 2-cyclohexanediamine (5.3 mg, 0.04 mmol) in 1,4-dioxane (0.5 mL) was degassed with N₂ for three times, then reached at 80° C. until TLC showed the reaction was complete. After cooling down, the reaction mixture was diluted with ethyl acetate (20 mL), washed with ammonium hydroxide (20 mL*2) and water (20 mL). The organic layer was dried over Na₂SO₄, concentrated to dryness in vacuum and purified by column chromatography to give product 19k (42 mg, yield: 58.1%).

The preparation of compounds 19a-19w refer to the synthesis of compound 17 in example 17, compound 18 in example 18 or compound 19a and 19k in example 19.

TABLE 5

| | | MS | |
|---|---|---|---|
| | | [M + | |
| Cpds | Structure | 1]⁺ | ¹H NMR |
| 19a | 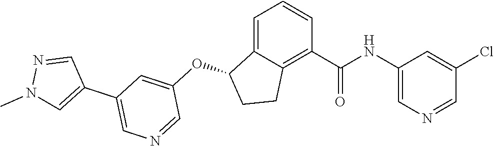 [SMILES: Cn1cc(-c2cncc(O[C@H]3CCc4c(C(=O)Nc5cncc(Cl)c5)cccc43)c2)cn1] | 446.1 | ¹H NMR (62 MHz, acetone-d₆) δ 9.87 (s, 1H), 8.87-8.84 (m, 1H), 8.48-7.28 (m, 10H), 6.14- 5.97 (m, 1H), 3.94 (s, 3H), 3.44-3.24 (m, 2H), 2.78-2.22 (m, 2H). |
| 19b | 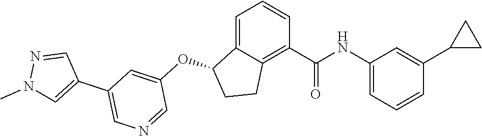 [SMILES: Cn1cc(-c2cncc(O[C@H]3CCc4c(C(=O)Nc5cccc(C6CC6)c5)cccc43)c2)cn1] | 451.2 | ¹H NMR (400 MHz, DMSO-d₆) δ 10.17 (s, 1H), 8.47 (d, J = 1.7 Hz, 1H), 8.31 (s, 1H), 8.19 (d, J = 2.7 Hz, 1H), 8.03 (s, 1H), 7.75-7.67 (m, 2H), 7.61 (d, J = 7.5 Hz, 1H), 7.59-7.52 (m, 1H), 7.49 (t, J = 1.9 Hz, 1H), 7.42 (t, J = 7.6 Hz, 1H), 7.21 (t, J = 7.9 Hz, 1H), 6.83 (d, J = 7.7 Hz, 1H), 6.07 (dd, J = 6.6, 3.7 Hz, 1H), 3.89 (s, 3H), 3.28 (t, J = 7.3 Hz, 1H), 3.19- 3.06 (m, 1H), 2.70-2.56 (m, 2H), 2.17-2.04 |
| | | | (m, 2H), 1.97-1.85 (m, 2H), 1.01-0.93 (m, |
| | | | 2H), 0.68-0.62 (m, 2H). |
| 19c | 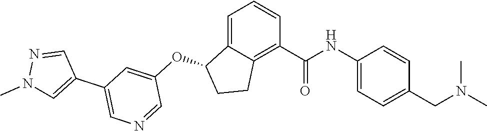 | 468.2 | ¹H NMR (400 MHz, DMSO-d₆) δ 8.47 (d, J = 1.6 Hz, 1H), 8.31 (s, 1H), 8.19 (d, J = 2.7 Hz, 1H), 8.03 (s, 1H), 7.74-7.68 (m, 4H), 7.62 (d, J = 7.5 Hz, 1H), 7.42 (t, J = 7.6 Hz, 1H), 7.27 (d, J = 8.2 Hz, 2H), 6.07 (dd, J = 6.6, 3.7 Hz, 1H), 3.89 (s, 3H), 3.27 (d, J = 6.3 Hz, 1H), 3.19-3.06 (m, 1H), 2.68-2.60 (m, 1H), 2.19 (s, 6H), 2.17-2.05 (m, 2H). |
| 19d | 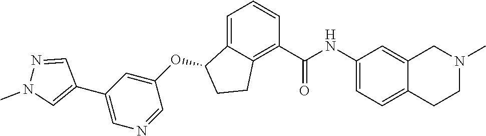 | 480.2 | ¹H NMR (400 MHz, DMSO-d₆) δ 10.17 (s, 1H), 8.47 (d, J = 1.6 Hz, 1H), 8.31 (s, 1H), 8.19 (d, J = 2.7 Hz, 1H), 8.03 (s, 1H), 7.73-7.67 (m, 2H), 7.61 (d, J = 7.5 Hz, 1H), 7.54-7.45 (m, 2H), 7.41 (t, J = 7.6 Hz, 1H), 7.09 (d, J = 8.3 Hz, 1H), 6.07 (dd, J = 6.6, 3.7 Hz, 1H), 3.89 (s, 3H), 3.54 (s, 2H), 3.30-3.25 (m, 1H), 3.20-3.05 (m, 1H), 2.80 (t, J = 5.8 Hz, 2H), 2.69-2.57 (m, 3H), 2.39 (s, 3H), 2.16-2.04 |
| | | | (m, 2H). |
| 19e | 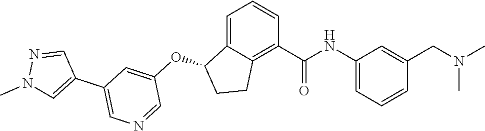 | 468.2 | ¹H NMR (400 MHz, DMSO-d₆) δ 10.26 (s, 1H), 8.47 (d, J = 1.6 Hz, 1H), 8.31 (s, 1H), 8.19 (d, J = 2.7 Hz, 1H), 8.03 (s, 1H), 7.78 (s, 1H), 7.73-7.70 (m, 2H), 7.63 (dd, J = 10.1, 7.5 Hz, 2H), 7.42 (t, J = 7.6 Hz, 1H), 7.29 (t, J = 7.8 Hz, 1H), 7.03 (d, J = 7.6 Hz, 1H), 6.07 (dd, J = 6.7, 3.7 Hz, 1H), 3.89 (s, 3H), 3.42 (s, 2H), 3.28 (d, J = 7.7 Hz, 1H), 3.20-3.07 (m, 2H), 2.68-2.56 (m, 1H), 2.19 (s, 6H), 2.17- |
| | | | 2.04 (m, 2H). |
| 19f | 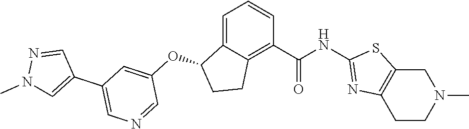 | 487.2 | ¹H NMR (400 MHz, DMSO-d₆) δ 12.42 (s, 1H), 8.47 (d, J = 1.6 Hz, 1H), 8.31 (s, 1H), 8.19 (d, J = 2.7 Hz, 1H), 8.02 (s, 1H), 7.86 (d, J = 7.6 Hz, 1H), 7.71 (d, J = 2.3 Hz, 1H), 7.64 (d, J = 7.5 Hz, 1H), 7.41 (t, J = 7.6 Hz, 1H), 6.07 (dd, J = 6.7, 3.8 Hz, 1H), 3.89 (s, 3H), 3.52 (s, 2H), 3.25-3.14 (m, 2H), 2.71 (d, J = 5.7 Hz, 4H), 2.66-2.58 (m, 1H), 2.39 (s, 3H), 2.10 (tt, J = 8.8, 4.4 Hz, 1H). |
| 19g | 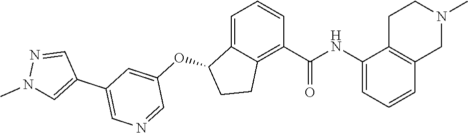 | 480.2 | ¹H NMR (400 MHz, DMSO-d₆) δ 9.69 (s, 1H), 8.47 (d, J = 1.7 Hz, 1H), 8.31 (s, 1H), 8.19 (d, J = 2.7 Hz, 1H), 8.03 (s, 1H), 7.75 (d, J = 7.5 Hz, 1H), 7.71 (t, J = 2.2 Hz, 1H), 7.61 (d, J = 7.6 Hz, 1H), 7.42 (t, J = 7.6 Hz, 1H), 7.27 (d, J = 7.8 Hz, 1H), 7.16 (t, J = 7.7 Hz, 1H), 6.97 (d, J = 7.6 Hz, 1H), 6.07 (dd, J = 6.6, 3.7 Hz, 1H), 3.89 (s, 3H), 3.51 (s, 2H), 3.23-3.13 (m, 1H), 2.80 (t, J = 6.0 Hz, 2H), |
| | | | 2.65 (d, J = 7.4 Hz, 1H), 2.59 (t, J = 6.1 Hz, |
| | | | 3H), 2.34 (s, 3H), 2.16-2.06 (m, 1H). |
| 19h | 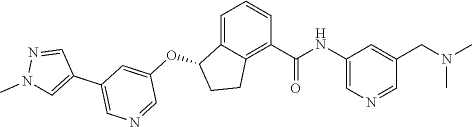 | 469.2 | ¹H NMR (400 MHz, DMSO-d₆) δ 10.49 (s, 1H), 8.80 (s, 1H), 8.48 (s, 1H), 8.31 (s, 1H), 8.21 (s, 3H), 8.03 (s, 1H), 7.80-7.63 (m, 3H), 7.44 (t, J = 7.6 Hz, 1H), 6.08 (s, 1H), 3.89 (s, 3H), 3.42 (s, 2H), 3.32 (d, J = 8.6 Hz, 1H), 3.17 (d, J = 8.1 Hz, 1H), 2.65 (dd, J = 14.0, 6.9 Hz, 1H), 2.17 (s, 7H). |
| 19i | 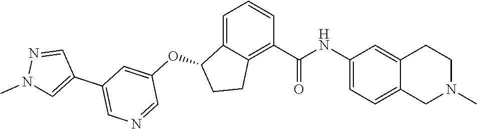 | 480.2 | ¹H NMR (400 MHz, DMSO-d₆) δ 10.16 (s, 1H), 8.47 (d, J = 1.6 Hz, 1H), 8.31 (s, 1H), 8.19 (d, J = 2.7 Hz, 1H), 8.03 (s, 1H), 7.73- 7.66 (m, 2H), 7.62-7.57 (m, 2H), 7.49-7.37 (m, 2H), 7.02 (d, J = 8.3 Hz, 1H), 6.07 (dd, J = 6.6, 3.8 Hz, 1H), 3.89 (s, 3H), 3.47 (s, 2H), 3.27 (t, J = 7.3 Hz, 1H), 3.16-3.07 (m, 1H), 2.82 (t, J = 6.0 Hz, 2H), 2.69-2.63 (m, 1H), 2.63- |
| | | | 2.58 (m, 2H), 2.35 (s, 3H), 2.15-2.05 (m, 1H). |
| 19j | 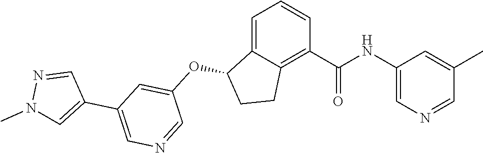 | 426.2 | ¹H NMR (400 MHz, DMSO-d₆) δ 10.43 (s, 1H), 8.70 (d, J = 2.3 Hz, 1H), 8.47 (d, J = 1.6 Hz, 1H), 8.31 (s, 1H), 8.23-8.14 (m, 2H), 8.04 (d, J = 13.5 Hz, 2H), 7.78-7.69 (m, 2H), 7.64 (d, J = 7.5 Hz, 1H), 7.44 (t, J = 7.6 Hz, 1H), 6.08 (dd, J = 6.7, 3.7 Hz, 1H), 3.89 (s, 3H), 3.29 (dd, J = 8.2, 6.2 Hz, 1H), 3.14 (ddd, J = 17.0, 8.6, 5.0 Hz, 1H), 2.64 (dq, J = 14.2, 6.7 Hz, 1H), 2.32 (s, 3H), 2.11 (ddt, J = 13.1, 8.5, 4.5 Hz, 1H). |
| 19k | 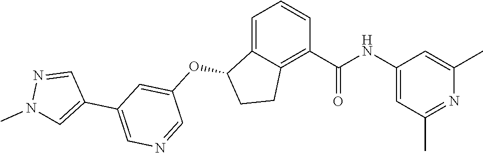 | 440.2 | ¹H NMR (400 MHz, DMSO-d₆) δ 10.48 (s, 1H), 8.48 (s, 1H), 8.31 (s, 1H), 8.19 (d, J = 2.6 Hz, 1H), 8.03 (s, 1H), 7.72 (d, J = 7.8 Hz, 2H), 7.65 (d, J = 7.5 Hz, 1H), 7.44 (d, J = 2.6 Hz, 3H), 6.08 (dd, J = 6.6, 3.7 Hz, 1H), 3.89 (s, 3H), 3.28 (t, J = 7.4 Hz, 1H), 3.18-3.07 (m, 1H), 2.70-2.56 (m, 2H), 2.40 (s, 6H), 2.17-2.05 (m, 1H). |
| 19l | 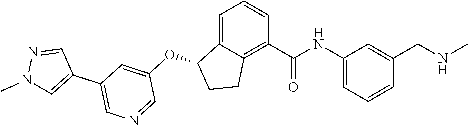 | 454.2 | ¹H NMR (400 MHz, DMSO-d₆) δ 10.26 (s, 1H), 8.49 (s, 1H), 8.31 (s, 1H), 8.21 (s, 1H), 8.04 (s, 1H), 7.80-7.61 (m, 5H), 7.42 (t, J = 7.5 Hz, 1H), 7.28 (t, J = 7.6 Hz, 1H), 7.06 (d, J = 7.1 Hz, 1H), 6.08 (s, 1H), 3.89 (s, 3H), 3.64 (s, 2H), 3.34 (d, J = 16.0 Hz, 1H), 2.65 (d, J = 13.2 Hz, 1H), 2.29 (s, 3H), 2.16-2.06 (m, 1H). |
| 19m | 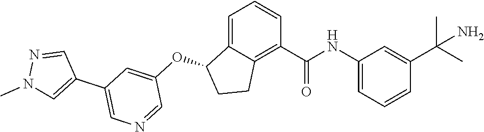 [SMILES: Cn1cc(-c2cncc(O[C@H]3CCc4c(C(=O)Nc5cccc(C(C)(C)N)c5)cccc43)c2)cn1] | 468.2 | ¹H NMR (400 MHz, DMSO-d₆) δ 9.49 (s, 1H), 8.46 (s, 1H), 8.20 (s, 1H), 8.13 (s, 1H), 7.99 (s, 1H), 7.92 (s, 1H), 7.78 (t, J = 7.5 Hz, 2H), 7.67-7.58 (m, 2H), 7.42-7.25 (m, 3H), 6.03 (d, J = 10.3 Hz, 1H), 3.93 (s, 3H), 3.48- 3.38 (m, 1H), 3.23 (dd, J = 17.0, 5.6 Hz, 1H), 2.72 (s, 1H), 2.20 (s, 1H), 1.46 (s, 6H). |
| 19n | 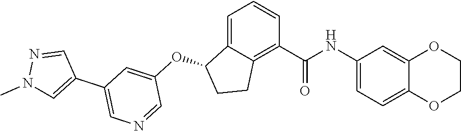 [SMILES: Cn1cc(-c2cncc(O[C@H]3CCc4c(C(=O)Nc5ccc6c(c5)OCCO6)cccc43)c2)cn1] | 469.2 | ¹H NMR (400 MHz, DMSO-d₆) δ 10.10 (s, 1H), 8.47 (d, J = 1.6 Hz, 1H), 8.31 (s, 1H), 8.18 (d, J = 2.6 Hz, 1H), 8.02 (s, 1H), 7.70 (t, J = 2.2 Hz, 1H), 7.66 (d, J = 7.6 Hz, 1H), 7.60 (d, J = 7.4 Hz, 1H), 7.45-7.35 (m, 2H), 7.18 (dd, J = 8.7, 2.4 Hz, 1H), 6.82 (d, J = 8.8 Hz, 1H), 6.06 (dd, J = 6.7, 3.7 Hz, 1H), 4.23 (h, J = 3.5, 2.8 Hz, 4H), 3.89 (s, 3H), 3.28-3.22 (m, 1H), 3.15-3.06 (m, 1H), 2.62 (dq, J = 14.2, |
| | | | 7.0 Hz, 1H), 2.09 (ddt, J = 13.1, 8.6, 4.5 |
| | | | Hz, 1H). |
| 19o | 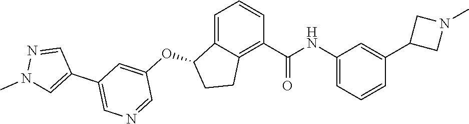 | 480.2 | ¹H NMR (400 MHz, DMSO-d₆) δ 10.27 (s, 1H), 8.48 (s, 1H), 8.31 (s, 1H), 8.20 (s, 1H), 8.03 (s, 1H), 7.83 (s, 1H), 7.73-7.60 (m, 4H), 7.41 (d, J = 7.3 Hz, 1H), 7.30 (d, J = 7.6 Hz, 1H), 7.08 (d, J = 7.2 Hz, 1H), 6.07 (s, 1H), 3.89 (s, 4H), 3.14 (d, J = 16.7 Hz, 1H), 2.84 (s, 1H), 2.61 (s, 1H), 2.24 (s, 4H), 2.11 (d, J = 15.9 Hz, 1H), 1.93 (d, J = 17.7 Hz, 1H). |
| 19p | 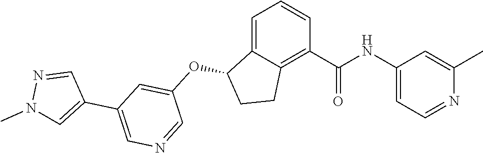 | 426.2 | ¹H NMR (400 MHz, DMSO-d₆) δ 10.56 (s, 1H), 8.48 (d, J = 1.6 Hz, 1H), 8.34 (d, J = 5.6 Hz, 1H), 8.31 (s, 1H), 8.19 (d, J = 2.8 Hz, 1H), 8.03 (s, 1H), 7.75-7.70 (m, 2H), 7.67-7.63 (m, 2H), 7.54 (dd, J = 5.7, 1.9 Hz, 1H), 7.45 (t, J = 7.6 Hz, 1H), 6.08 (dd, J = 6.6, 3.7 Hz, 1H), 3.89 (s, 3H), 3.33-3.26 (m, 1H), 3.19- 3.06 (m, 1H), 2.69-2.59 (m, 1H), 2.45 (s, 3H), 2.17-2.04 (m, 2H). |
| 19q | 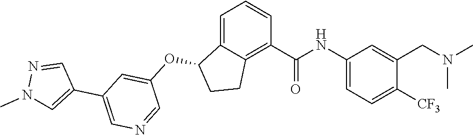 | 536.2 | ¹H NMR (400 MHz, DMSO-d₆) δ 10.61 (s, 1H), 8.48 (d, J = 1.6 Hz, 1H), 8.31 (s, 1H), 8.19 (d, J = 2.7 Hz, 1H), 8.17 (s, 1H), 8.03 (s, 1H), 7.92 (d, J = 8.7 Hz, 1H), 7.77 (d, J = 7.6 Hz, 1H), 7.71 (t, J = 2.2 Hz, 1H), 7.66 (dd, J = 14.2, 8.1 Hz, 2H), 7.44 (t, J = 7.6 Hz, 1H), 6.08 (dd, J = 6.6, 3.7 Hz, 1H), 3.89 (s, 3H), 3.53 (s, 2H), 3.32-3.24 (m, 1H), 3.20-3.10 (m, 1H), 2.63 (dt, J = 14.3, 7.6 Hz, 1H), 2.22 (s, 6H), 2.11 (tt, J = 8.6, 4.4 Hz, 1H). |
| 19r | 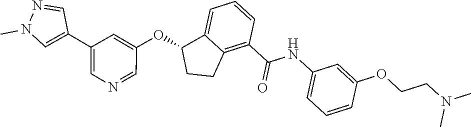 | 498.2 | ¹H NMR (400 MHz, DMSO-d₆) δ 10.24 (s, 1H), 8.47 (d, J = 1.6 Hz, 1H), 8.31 (s, 1H), 8.19 (d, J = 2.6 Hz, 1H), 8.03 (s, 1H), 7.73- 7.67 (m, 2H), 7.62 (d, J = 7.5 Hz, 1H), 7.43 (dd, J = 15.2, 7.7 Hz, 2H), 7.33 (d, J = 8.3 Hz, 1H), 7.24 (t, J = 8.1 Hz, 1H), 6.69 (dd, J = 8.3, 2.4 Hz, 1H), 6.07 (dd, J = 6.7, 3.7 Hz, 1H), 4.04 (t, J = 5.8 Hz, 2H), 3.89 (s, 3H), 3.19- |
| | | | 3.06 (m, 1H), 2.63 (t, J = 5.7 Hz, 3H), 2.26- |
| | | | 2.20 (m, 14H), 2.17-2.05 (m, 1H). |
| 19s | 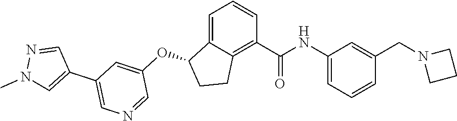 [SMILES: Cn1cc(-c2cncc(O[C@H]3CCc4c(C(=O)Nc5cccc(CN6CCC6)c5)cccc43)c2)cn1] | 480.2 | ¹H NMR (400 MHz, DMSO-d₆) δ 10.25 (s, 1H), 8.48 (s, 1H), 8.31 (s, 1H), 8.20 (s, 1H), 8.03 (s, 1H), 7.75-7.59 (m, 5H), 7.42 (t, J = 7.4 Hz, 1H), 7.26 (t, J = 7.7 Hz, 1H), 6.98 (d, J = 7.2 Hz, 1H), 6.07 (s, 1H), 3.89 (s, 3H), 3.50 (s, 2H), 3.28 (d, J = 6.8 Hz, 1H), 3.13 (t, J = 6.6 Hz, 5H), 2.68-2.60 (m, 1H), 2.15-2.07 (m, 1H), 1.99 (d, J = 6.6 Hz, 2H). |
| 19t | 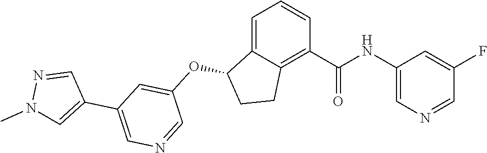 [SMILES: Cn1cc(-c2cncc(O[C@H]3CCc4c(C(=O)Nc5cncc(F)c5)cccc43)c2)cn1] | 430.2 | ¹H NMR (400 MHz, DMSO-d₆) δ 10.75 (s, 1H), 8.76 (d, J = 1.8 Hz, 1H), 8.48 (d, J = 1.6 Hz, 1H), 8.34 (d, J = 2.6 Hz, 1H), 8.31 (s, 1H), 8.21 (dd, J = 10.5, 2.5 Hz, 2H), 8.03 (s, 1H), 7.78 (d, J = 7.6 Hz, 1H), 7.71 (t, J = 2.3 Hz, 1H), 7.67 (d, J = 7.5 Hz, 1H), 7.46 (t, J = 7.6 Hz, 1H), 6.08 (dd, J = 6.7, 3.7 Hz, 1H), 3.89 (s, 3H), 3.31 (t, J = 7.3 Hz, 1H), 3.15 (ddd, J = 17.0, 8.5, 4.9 Hz, 1H), 2.64 (dq, J = 14.2, 6.3 Hz, 1H), 2.11 (ddt, J = 13.1, 8.6, 4.4 Hz, |
| | | | 1H). |
| 19u | 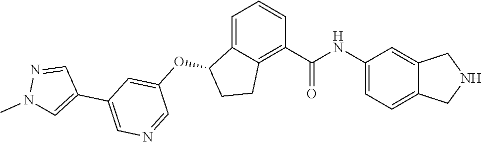 [SMILES: Cn1cc(-c2cncc(O[C@H]3CCc4c(C(=O)Nc5ccc6c(c5)CNC6)cccc43)c2)cn1] | 452.2 | ¹H NMR (400 MHz, DMSO-d₆) δ 10.23 (s, 1H), 8.47 (d, J = 1.6 Hz, 1H), 8.31 (s, 1H), 8.19 (d, J = 2.7 Hz, 1H), 8.03 (s, 1H), 7.76- 7.68 (m, 3H), 7.61 (d, J = 7.5 Hz, 1H), 7.52 (d, J = 8.1 Hz, 1H), 7.42 (t, J = 7.6 Hz, 1H), 7.22 (d, J = 8.1 Hz, 1H), 6.07 (dd, J = 6.7, 3.7 Hz, 1H), 4.08 (d, J = 12.0 Hz, 4H), 3.93-3.87 (m, 3H), 3.27 (d, J = 7.9 Hz, 2H), 3.19-3.06 (m, 1H), 2.69-2.60 (m, 1H), 2.16-2.05 (m, 1H). |
| 19v | 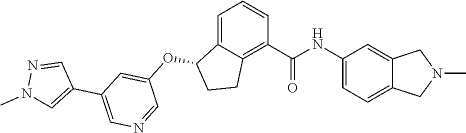 | 466.2 | ¹H NMR (400 MHz, DMSO-d₆) δ 10.23 (s, 1H), 8.47 (s, 1H), 8.31 (s, 1H), 8.19 (d, J = 2.8 Hz, 1H), 8.03 (s, 1H), 7.70 (d, J = 9.5 Hz, 3H), 7.61 (d, J = 7.6 Hz, 1H), 7.50 (d, J = 8.1 Hz, 1H), 7.42 (t, J = 7.7 Hz, 1H), 7.19 (d, J = 8.1 Hz, 1H), 6.13-6.04 (m, 1H), 3.89 (s, 3H), 3.80 (d, J = 13.1 Hz, 4H), 3.28 (s, 1H), 3.19-3.06 (m, 1H), 2.70-2.58 (m, 1H), 2.49 (s, 3H), 2.19-2.04 (m, 2H). |
| 19w | 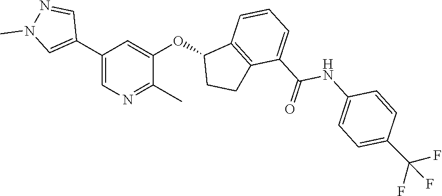 [SMILES: Cc1ncc(-c2cnn(C)c2)cc1O[C@H]1CCc2c(C(=O)Nc3ccc(C(F)(F)F)cc3)cccc21] | 493.2 | ¹H NMR (400 MHz, DMSO-d₆) δ 10.66 (s, 1H), 8.38-8.22 (m, 2H), 8.01 (s, 2H), 7.98 (s, 1H), 7.76-7.3 (m, 4H), 7.64 (d, J = 7.5 Hz, 1H), 7.46 (t, J = 7.6 Hz, 1H), 6.04-6.02 m, 1H), 3.89 (s, 3H), 3.32-3.29 (m, 1H), 3.19-3.11 (m, 1H), 2.75-2.67 (m, 1H), 2.27 (s, 3H), 2.10-2.05 (m, 1H). |

### Example 20

### (S)-1-(methyl (5-(1-methyl-1H-pyrazol-4-yl) pyridin-3-yl)amino)-N-(4-(trifluoromethyl)phenyl)-2,3-dihydro-1H-indene-4-carboxamide

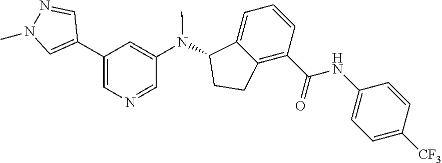 [SMILES: CN(c1cncc(-c2cnn(C)c2)c1)[C@H]1CCc2c(C(=O)Nc3ccc(C(F)(F)F)cc3)cccc21]

Compound 20 was prepared by the following steps:

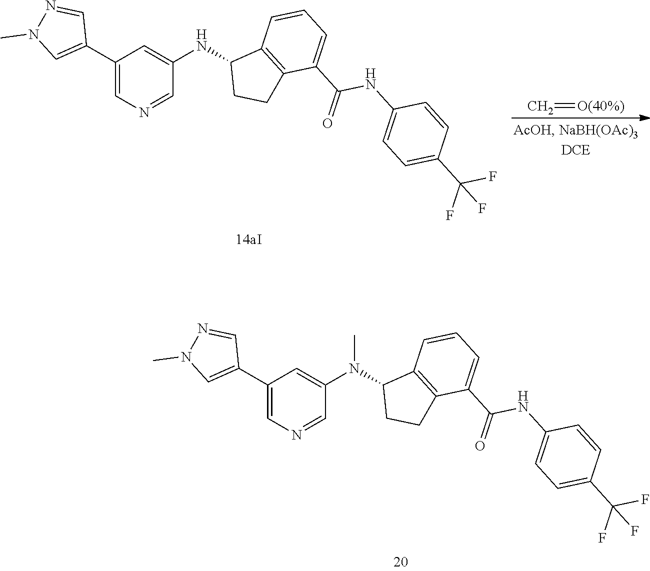

40% formalin (188 mg, 2.5 mmol) and sodium cyanoborohydride (158 mg, 2.5 mmol) were sequentially added to a solution of 14al (120 mg, 0.25 mmol) and acetic acid (0.2 mL) in 1,2-dichloroethane (2 mL) at 3 h intervals at 0° C., and the process was repeated three times. After each addition, the reaction mixture was allowed to warm to room temperature and stirred until the next addition of materials. The reaction was stopped while the product was not further formed with the addition by TLC monitoring. The reaction mixture was concentrated to dryness in vacuum and purified by column chromatography to give the desired product 20 as a solid (38 mg, yield: 31.0%). ESI-MS (m/z): 492.2 [M+H]⁺. ¹H NMR (400 MHz, DMSO-d₆) 10.64 (s, 1H), 8.25 (s, 1H), 8.20 (s, 1H), 8.17 (s, 1H), 8.06-7.93 (m, 3H), 7.73 (d, J=8.5 Hz, 2H), 7.67 (d, J=7.4 Hz, 1H), 7.48 (s, 1H), 7.40 (t, J=7.5 Hz, 1H), 7.33 (d, J=7.4 Hz, 1H), 5.79-5.75 (m, 1H), 3.88 (s, 3H), 3.30-3.22 (m, 1H), 3.13-3.05 (m, 1H), 2.66 (s, 3H), 2.50-2.38 (m, 1H), 2.04-1.89 (m, 1H).

### Example 21

### (S)—N-(5-chloropyridin-3-yl)-1-(methyl (5-(1-methyl-1H-pyrazol-4-yl) pyridin-3-yl)amino)-2,3-dihydro-1H-indene-4-carboxamide

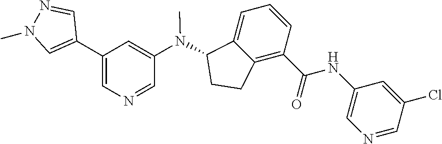 [SMILES: CN(c1cncc(-c2cnn(C)c2)c1)[C@H]1CCc2c(C(=O)Nc3cncc(Cl)c3)cccc21]

Compound 21 was prepared by the following steps:

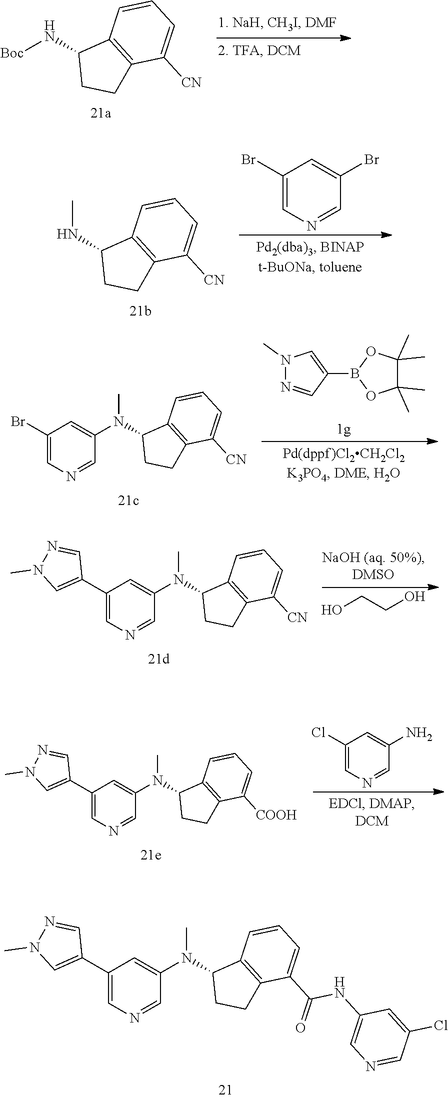

Step 1: To a stirred solution of 21a (5.16 g, 20.0 mmol) in DMF (25 mL) was added NaH (60%, 880 mg, 22.0 mmol) at 0° C. in portions, followed by addition of Mel (3.55 g, 25.0 mmol) after being stirred for 15 min. The resulting mixture was stirred at 25° C. until TLC analysis showed the reaction was complete. The reaction mixture was quenched by the addition of water (100 mL) with stirring, then extracted with EtOAc (100 mL*2). The combined organic layers were washed with brine (100 mL*2), dried over Na₂SO₄, and concentrated to dryness under reduced pressure. A solution of the light yellow solid (4.5 g, 16.7 mmol) obtained above and CF₃COOH (10 mL) in CH₂Cl₂ (30 mL) was stirred at 25° C. until TLC analysis showed the reaction was complete, then diluted with CH₂Cl₂ (30 mL), and adjusted to pH>13 with 10% aqueous NaOH. The resulting mixture was stirred for a moment, then the aqueous layer was discarded, and the organic layer was washed with brine (50 mL), dried over Na₂SO₄, and concentrated to dryness under reduced pressure to give compound 21b as brown solid (2.6 g, yield: 91%).

Step 2: To a solution of 21b (2.6 g, 15.1 mmol), 3,5-dibromopyridine (5.4 g, 22.1 mmol) in toluene (25 mL) was added Pd₂(dba)₃ (274 mg, 0.3 mmol), BINAP (934 mg, 1.5 mmol) and t-BuONa (4.3 g, 45.3 mmol). The resulting mixture was degassed with N₂ for three times, then heated at 90° C. until TLC analysis showed the reaction was complete. After the mixture was cooled to 30° C., the mixture was diluted with EtOAc (75 mL), washed with water (100 mL), dried over Na₂SO₄, and concentrated to dryness under reduced pressure. The residue was purified on an ISCO chromatography to give compound 21c as brown thick oil (2.7 g, yield: 55%).

Step 3: To a solution of 21c (2.7 g, 8.2 mmol) and 1g (2.6 g, 12.3 mmol) in a mixture of solvent DME (12 mL) and water (6 mL) was added K₃PO₄ (3.0 g, 24.6 mmol) and Pd(dppf)Cl₂·CH₂Cl₂ (335 mg, 0.41 mmol). The resulting mixture was degassed with N₂ for three times, then stirred at 90° C. until TLC analysis showed the reaction was complete. After natural cooling to 30° C., the reaction mixture was allowed to stand and the aqueous phase was removed. The collected organic layer was concentrated to dryness under reduced pressure. The thick oil was redissolved in CH₂Cl₂ (30 mL), washed with water (30 mL), dried over Na₂SO₄, and concentrated to dryness under reduced pressure. The residue was purified on an ISCO chromatography to give compound 21d as yellow thick oil (1.8 g, yield: 67%).

Step 4: To a stirred solution of 21d (1.8 g, 5.5 mmol) in DMSO (9.0 mL) was added 50% aqueous NaOH (9.0 mL) and glycol (9.0 mL). The resulting mixture was heated to 110° C. and stirred until TLC analysis showed the reaction was complete. After natural cooling to 30° C., the reaction mixture was adjusted to pH=6˜7 with 1N HCl. The resulting precipitate was collected by filtration, washed with water (9.0 mL), and dried under vacuum at 50° C. to give compound 21e as light brown solid (1.6 g, yield: 88%).

Step 5: To a solution of 21e (57.7 mg, 0.17 mmol), 3-amino-5-chloropyridine (32.0 mg, 0.25 mmol) and DMAP (4 mg, 0.03 mmol) in CH₂Cl₂ (0.5 mL) was added EDCI (48 mg, 0.25 mmol). The resulting mixture was stirred at room temperature until TLC analysis showed the reaction was complete. The mixture was concentrated to dryness under reduced pressure and the residue was purified by chromatography to give compound 21 (54 mg, yield: 71%). ESI-MS (m/z): 459.2 [M+H]⁺. ¹H NMR (62 MHz, acetone-d₆) δ 9.88 (s, 1H), 8.87-8.83 (m, 1H), 8.52-8.49 (m, 1H), 8.35-8.31 (m, 1H), 8.21-8.18 (m, 2H), 8.05 (s, 1H), 7.86-7.34 (m, 5H), 5.88-5.62 (m, 1H), 3.92 (s, 3H), 3.46-3.25 (m, 2H), 2.73 (s, 3H), 2.68-2.21 (m, 2H).

### Example 22

### (S)—N-(3-cyclopropylphenyl)-1-(methyl (5-(1-methyl-1H-pyrazol-4-yl) pyridin-3-yl)amino)-2,3-dihydro-1H-indene-4-carboxamide

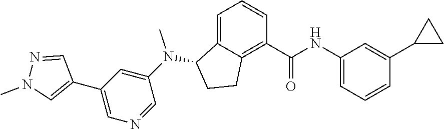 [SMILES: CN(c1cncc(-c2cnn(C)c2)c1)[C@H]1CCc2c(C(=O)Nc3cccc(C4CC4)c3)cccc21]

Compound 22a was prepared by the following steps:

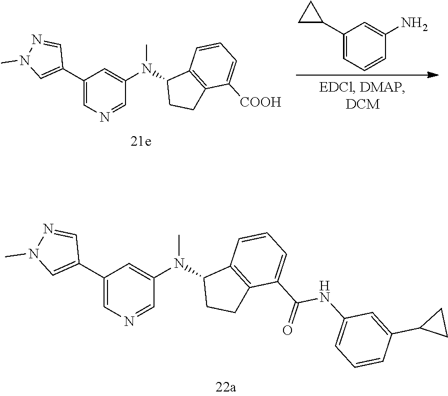 [SMILES: CN(c1cncc(-c2cnn(C)c2)c1)[C@H]1CCc2c(C(=O)O)cccc21 ; Nc1cccc(C2CC2)c1 ; CN(c1cncc(-c2cnn(C)c2)c1)[C@H]1CCc2c(C(=O)Nc3cccc(C4CC4)c3)cccc21]

To a solution of 21e (57.7 mg, 0.17 mmol), 3-Cyclopropylaniline (33.3 mg, 0.25 mmol) and DMAP (4 mg, 0.03 mmol) in CH₂Cl₂ (0.5 mL) was added EDCI (48 mg, 0.25 mmol). The resulting mixture was stirred at room temperature until TLC analysis showed the reaction was complete. The mixture was concentrated to dryness under reduced pressure and the residue was purified by chromatography to give compound 22a (51 mg, yield: 65%).

The preparation of compounds 22a-22g refer to the synthesis of compound 20 in example 20 or compound 21 in example 21

TABLE 6

| | | MS | |
|---|---|---|---|
| | | [M + | |
| Cpds | Structure | 1]⁺ | ¹H NMR |
| 22a | 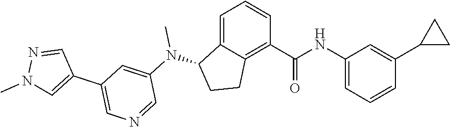 [SMILES: CN(c1cncc(-c2cnn(C)c2)c1)[C@H]1CCc2c(C(=O)Nc3cccc(C4CC4)c3)cccc21] | 464.2 | ¹H NMR (400 MHz, DMSO-d₆) δ 10.16 (s, 1H), 8.27-8.12 (m, 3H), 7.97 (s, 1H), 7.61 (d, J = 7.5 Hz, 1H), 7.57-7.45 (m, 3H), 7.37 (t, J = 7.5 Hz, 1H), 7.29 (d, J = 7.5 Hz, 1H), 7.21 (t, J = 7.9 Hz, 1H), 6.83 (dt, J = 7.7, 1.3 Hz, 1H), 5.76 (t, J = 7.9 Hz, 1H), 3.88 (s, 3H), 3.25 (dq, J = 13.7, 6.1, 4.6 Hz, 1H), 3.07 (dt, J = 16.9, 8.5 Hz, 1H), 2.66 (s, 3H), 2.42 (ddd, J = 11.8, 8.1, 3.5 Hz, 1H), 2.00- |
| | | | 1.88 (m, 2H), 0.99-0.92 (m, 2H), 0.69-0.63 (m, |
| | | | 2H). |
| 22b | 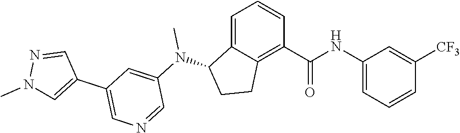 [SMILES: CN(c1cncc(-c2cnn(C)c2)c1)[C@H]1CCc2c(C(=O)Nc3cccc(C(F)(F)F)c3)cccc21] | 492.2 | ¹H NMR (400 MHz, DMSO-d₆) δ 10.60 (s, 1H), 8.26 (d, J = 7.1 Hz, 2H), 8.18 (dd, J = 9.3, 2.2 Hz, 2H), 8.01 (dt, J = 8.2, 1.4 Hz, 1H), 7.97 (s, 1H), 7.68 (d, J = 7.5 Hz, 1H), 7.61 (t, J = 8.0 Hz, 1H), 7.49-7.45 (m, 2H), 7.41 (t, J = 7.5 Hz, 1H), 7.33 (d, J = 7.5 Hz, 1H), 5.77 (t, J = 7.9 Hz, 1H), 3.88 (s, 3H), 3.32-3.24 (m, IH), 3.13-3.05 (m, 1H), 2.66 (s, 3H), 2.44 (ddt, J = 11.7, 8.3, 4.1 Hz, 1H), 1.97 (dq, J = 12.8, 8.8 Hz, 1H). |
| 22c | 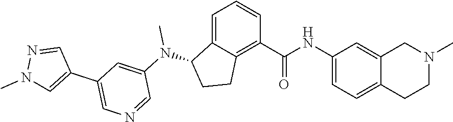 | 493.3 | ¹H NMR (400 MHz, DMSO-d₆) δ 10.14 (s, 1H), 8.25 (s, 1H), 8.17 (d, J = 9.0 Hz, 2H), 7.97 (s, 1H), 7.60 (d, J = 7.4 Hz, 1H), 7.49 (d, J = 8.9 Hz, 3H), 7.37 (t, J = 7.5 Hz, 1H), 7.28 (d, J = 7.5 Hz, 1H), 7.07 (d, J = 8.2 Hz, 1H), 5.75 (t, J = 7.8 Hz, 1H), 3.88 (s, 3H), 3.47 (s, 2H), 3.24 (dd, J = 19.0, 8.8 Hz, 1H), 3.06 (dt, J = 16.8, 8.4 Hz, 1H), 2.78 (t, J = 5.5 Hz, 2H), 2.66 (s, 3H), |
| | | | 2.59 (d, J = 5.7 Hz, 2H), 2.42 (dd, J = 16.2, 4.7 |
| | | | Hz, 1H), 2.34 (s, 3H), 2.00-1.91 (m, 1H). |
| 22d | 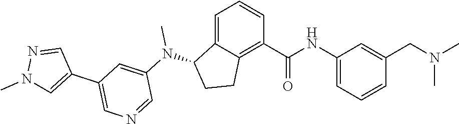 | 481.3 | ¹H NMR (400 MHz, DMSO-d₆) δ 10.23 (s, 1H), 8.25 (s, 1H), 8.17 (d, J = 10.7 Hz, 2H), 7.97 (s, 1H), 7.76 (s, 1H), 7.64 (t, J = 6.9 Hz, 2H), 7.48 (s, 1H), 7.39 (s, 1H), 7.29 (d, J = 6.0 Hz, 2H), 7.02 (d, J = 7.4 Hz, 1H), 5.78 (s, 1H), 3.88 (s, 3H), 3.29-3.22 (m, 1H), 3.10-3.04 (m, 1H), 2.66 (s, 3H), 2.46-2.38 (m, 1H), 2.16 (s, 6H), 1.96 (d, J = 21.1 Hz, 1H). |
| 22e | 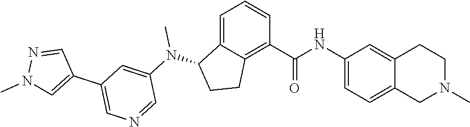 | 493.3 | ¹H NMR (400 MHz, DMSO-d₆) δ 10.13 (s, 1H), 8.25 (s, 1H), 8.22-8.11 (m, 2H), 7.96 (s, 1H), 7.64-7.53 (m, 2H), 7.46 (d, J = 10.0 Hz, 2H), 7.37 (t, J = 7.6 Hz, 1H), 7.28 (d, J = 7.5 Hz, 1H), 7.01 (d, J = 8.3 Hz, 1H), 5.75 (t, J = 7.8 Hz, 1H), 3.88 (s, 3H), 3.45 (s, 2H), 3.28-3.19 (m, 1H), 3.06 (dt, J = 16.8, 8.6 Hz, 1H), 2.81 (d, J = 4.3 Hz, 2H), 2.66 (s, 3H), 2.59 (t, J = 6.0 Hz, 2H), 2.42 (td, J = |
| | | | 8.8, 8.3, 4.6 Hz, 1H), 2.34 (s, 3H), 2.03-1.90 (m, |
| | | | 1H). |
| 22f | 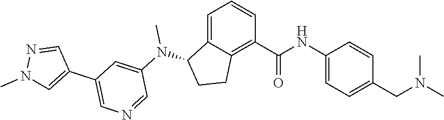 | 481.3 | ¹H NMR (400 MHz, DMSO-d₆) δ 10.24 (s, 1H), 8.25 (s, 1H), 8.17 (d, J = 8.8 Hz, 2H), 7.97 (s, 1H), 7.71 (d, J = 8.0 Hz, 2H), 7.62 (d, J = 7.3 Hz, 1H), 7.47 (s, 1H), 7.38 (t, J = 7.4 Hz, 1H), 7.31-7.23 (m, 3H), 5.76 (s, 1H), 3.88 (s, 3H), 3.26 (d, J = 10.0 Hz, 1H), 3.12-3.04 (m, 1H), 2.66 (s, 3H), 2.46-2.39 (m, 1H), 2.14 (s, 6H), 2.00-1.92 (m, 1H). |
| 22g | 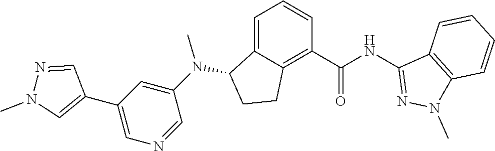 [SMILES: CN(c1cncc(-c2cnn(C)c2)c1)[C@H]1CCc2c(C(=O)Nc3nn(C)c4ccccc34)cccc21] | 478.3 | ¹H NMR (62 MHz, acetone-d₆) δ 9.95 (s, 1H), 8.28-8.11 (m, 2H), 8.10-8.00 (m, 1H), 7.99-7.76 (m, 3H), 7.76-7.52 (m, 1H), 7.51-7.28 (m, 4H), 7.27-6.84 (m, 1H), 5.75 (t, 1H), 3.95 (d, J = 5.1Hz, 6H), 3.66-3.15 (m, 2H), 2.73 (s, 3H), 2.65-2.14 (m, 1H), 1.93-1.68 (m, 1H). |

### Example 23

### (S)—N-(1-(methyl (5-(1-methyl-1H-pyrazol-4-yl) pyridin-3-yl)amino)-2,3-dihydro-1H-inden-4-yl)-4-(trifluoromethyl)benzamide

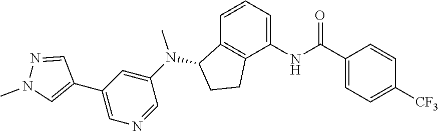 [SMILES: CN(c1cncc(-c2cnn(C)c2)c1)[C@H]1CCc2c(NC(=O)c3ccc(C(F)(F)F)cc3)cccc21]

Synthesis of compound 23 is similar procedures to compound 20.

ESI-MS (m/z): 492.2 [M+H]⁺. ¹H NMR (400 MHz, DMSO-d₆) δ 10.26 (s, 1H), 8.26-8.14 (m, 5H), 8.00-7.88 (m, 3H), 7.50-7.39 (m, 2H), 7.28 (t, J=7.6 Hz, 1H), 7.06-6.99 (m, 1H), 5.78 (t, J=7.7 Hz, 1H), 3.87 (s, 3H), 3.01-2.85 (m, 2H), 2.68 (s, 3H), 2.46-2.37 (m, 1H), 1.99-1.90 (m, 1H).

### Example 24

### (S)-2,2-difluoro-N-(1-((6-(1-methyl-1H-pyrazol-4-yl) pyrazin-2-yl)amino)-2,3-dihydro-1H-inden-4-yl)benzo[d][1,3]dioxole-5-carboxamide

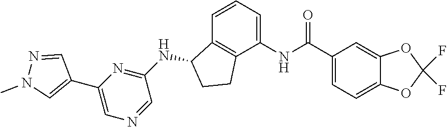 [SMILES: Cn1cc(-c2cncc(N[C@H]3CCc4c(NC(=O)c5ccc6c(c5)OC(F)(F)O6)cccc43)n2)cn1]

Compound 24 was prepared by the following steps:

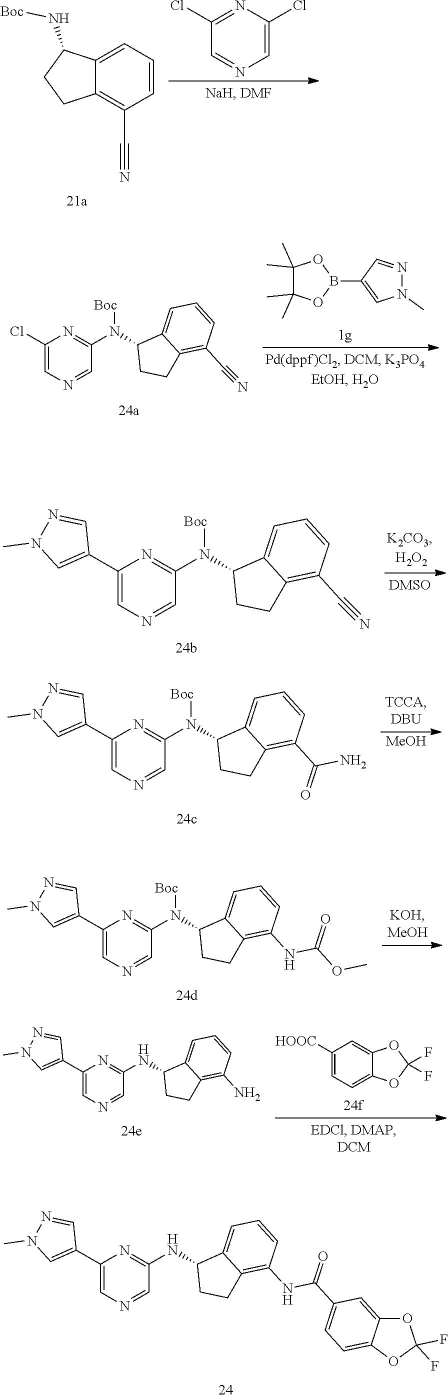

Step 1: To a stirred solution of 21a (5683 mg, 22.0 mmol) in DMF (15 mL) was added NaH (60%, 960 mg, 24.0 mmol) in portions at 0° C. The mixture was stirred for 30 min at 0° C., followed by addition of a solution of 2,6-dichloropyrazine (2979 mg, 20.0 mmol) in DMF (5 mL). After 1 h, TLC analysis showed the reaction was complete. The reaction mixture was quenched by addition of saturated aqueous NH₄Cl (10 mL), and extracted with EtOAc (20 mL*3). The combined organic phases were concentrated to dryness, and the residue was purified by chromatography to give product as a white solid (5340 mg, yield: 72%). ESI-MS (m/z): 371.1 [M+H]⁺.

Step 2: The solution of 24a (5340 mg, 14.4 mmol), 1g (4494 mg, 21.6 mmol), Pd(dppf)Cl₂·CH₂Cl₂ (1167 mg, 1.44 mmol) and K₃PO₄ (9170 mg, 43.2 mmol) in mixed solvent of EtOH (30 mL) and water (15 mL) was heated to 80° C. and reacted for 3 h under N₂ atmosphere. TLC analysis showed the reaction was complete. The separated organic phase was purified by chromatography to give product as a red brown solid (5098 mg, yield: 85%). ESI-MS (m/z): 417.2 [M+H]⁺.

Step 3: To a stirred solution of 24b (2915 mg, 7.00 mmol) in DMSO (15 mL) was added K₂CO₃ (2902 mg, 21.0 mmol) and H₂O₂ (30%, 7 mL). The resulting mixture was stirred at room temperature for 1 h. TLC analysis showed the reaction was complete. The mixture was diluted with water (30 mL) and extracted with EtOAc (30 mL*3). The combined organic phases were concentrated to dryness. The resulting residue was purified by chromatography to give product as a white solid (2616 mg, yield: 86%). ESI-MS (m/z): 435.2 [M+H]⁺.

Step 4: To a stirred solution of 24c (2171 mg, 5.00 mmol) in MeOH (10 mL) at 0° C. was added trichloroisocyanate (TCCA) (232 mg, 1.00 mmol) and DBU (1713 mg, 11.2 mmol). The resulting mixture was stirred at room temperature for 1 h. TLC analysis showed the reaction was complete, silica gel was added to the reaction mixture and the solvent was evaporated in vacuo. The residue was purified by chromatography to give product as a white solid (1641 mg, yield: 73%). ESI-MS (m/z): 465.2 [M+H]⁺.

Step 5: To a stirred solution of 24d (1535 mg, 3.5 mmol) in MeOH (10 mL) was added 50% aqueous KOH (5 mL). The mixture was reacted at 50° C. for 12 h. After TLC analysis showed the reaction was complete, the mixture was concentrated to remove MeOH, The residue was partitioned between CH₂Cl₂ (10 mL) and water (5 mL). The separated organic phase was purified by silica gel chromatography to give product as a white solid (933 mg, yield: 87%). ESI-MS (m/z): 307.2 [M+H]⁺.

Step 6: To a solution of 24e (31 mg, 0.10 mmol) and 24f (31 mg, 0.15 mmol) in CH₂Cl₂ (0.5 mL) was added DMAP (3 mg, 0.02 mmol) and EDCI (29 mg, 0.15 mmol) in turn. The resulting mixture was stirred at room temperature. After 5 h, TLC analysis showed the reaction was complete. The mixture was washed with 5% NaHCO₃ (0.5 mL), and purified by silica gel chromatography to give product as a light yellow solid (35 mg, yield: 71%). ESI-MS (m/z): 491.10 [M+H]⁺. ¹H NMR (62 MHz, acetone-d₆) δ 9.25 (s, 1H), 8.15 (s, 1H), 8.07 (s, 1H), 8.04-7.79 (m, 4H), 7.72-7.32 (m, 2H), 7.25 (s, 1H), 7.17 (s, 1H), 6.52 (d, J=8.4 Hz, 1H), 5.86-5.62 (m, 1H), 3.93 (s, 3H), 3.34-2.87 (m, 2H), 2.71-2.38 (m, 1H), 1.93-1.71 (m, 1H).

### Example 25

The preparation of compounds 25a-25f refer to the synthesis of compound 24 in example 24.

TABLE 7

| | | MS | |
|---|---|---|---|
| Cpds | Structure | [M + 1]⁺ | ¹H NMR |
| 25a | 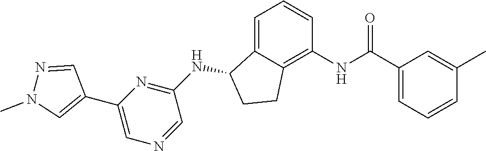 | 425.2 | ¹H NMR (62 MHz, acetone-d₆) δ 9.09 (s, 1H), 8.15 (s, 1H), 8.07 (s, 1H), 7.97 (s, 1H), 7.82 (s, 2H), 7.78-7.51 (m, 2H), 7.48-7.28 (m, 2H), 7.23 (s, 1H), 7.15 (s, 1H), 6.52 (d, J = 8.1 Hz, 1H), 5.79 (t, J = 7.5 Hz, 1H), 3.92 (s, 3H), 3.32-2.92 (m, 2H), 2.80-2.47 (m, 1H), 2.41 (s, 3H), 1.95-1.73 (m, 1H). |
| 25b | 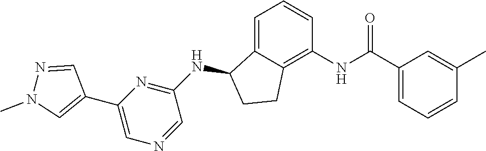 | 425.2 | ¹H NMR (62 MHz, acetone-d₆) δ 9.09 (s, 1H), 8.15 (s, 1H), 8.07 (s, 1H), 7.97 (s, 1H), 7.91-7.55 (m, 4H), 7.44-7.35 (m, 2H), 7.23 (s, 1H), 7.15 (s, 1H), 6.52 (d, J = 8.0 Hz, 1H), 6.02-5.50 (m, 1H), 3.93 (s, 3H), 3.24-2.91 (m, 2H), 2.75-2.49 (m, 1H), 2.42 (s, 3H), 1.96-1.71 (m, 1H). |
| 25c | 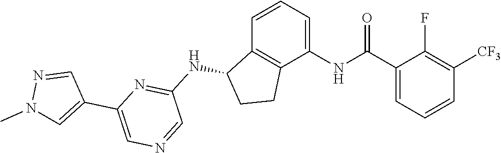 [SMILES: Cn1cc(-c2cncc(N[C@H]3CCc4c(NC(=O)c5cccc(C(F)(F)F)c5F)cccc43)n2)cn1] | 497.2 | ¹H NMR (62 MHz, acetone-d₆) δ 9.32 (s, 1H), 8.36-7.88 (m, 5H), 7.88-7.37 (m, 3H), 7.27 (s, 1H), 7.19 (s, 1H), 6.55 (d, J = 8.0 Hz, 1H), 6.07-5.62 (m, 1H), 3.93 (s, 3H), 3.29-2.95 (m, 2H), 2.79-2.46 (m, 1H), 1.96-1.63 (m, 1H). |
| 25d | 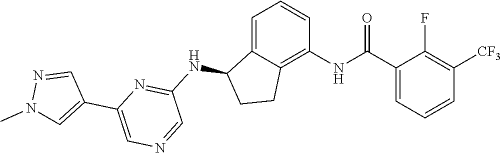 [SMILES: Cn1cc(-c2cncc(N[C@@H]3CCc4c(NC(=O)c5cccc(C(F)(F)F)c5F)cccc43)n2)cn1] | 497.2 | ¹H NMR (62 MHz, acetone-d₆) δ 9.34 (s, 1H), 8.40-7.89 (m, 5H), 7.88-7.39 (m, 3H), 7.27 (s, 1H), 7.19 (s, 1H), 6.55 (d, J = 8.0 Hz, 1H), 6.07-5.51 (m, 1H), 3.93 (s, 3H), 3.27-2.93 (m, 2H), 2.78-2.49 (m, 1H), 1.95-1.64 (m, 1H). |
| 25e | 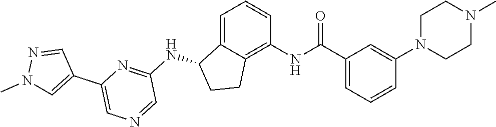 | 509.3 | ¹H NMR (62 MHz, acetone-d₆) δ 9.05 (s, 1H), 8.15-7.97 (m, 3H), 7.84 (s, 1H), 7.63-7.55 (m, 2H), 7.38-7.34 (m, 2H), 7.22-7.15 (m, 3H), 6.51 (d, J = 8.0 Hz, IH), 5.97-5.50 (m, 1H), 3.93 (s, 3H), 3.35-3.19 (m, 5H), 2.72-2.45 (m, 5H), 2.28 (s, 3H), 1.29-0.90 (m, 2H). |
| 25f | 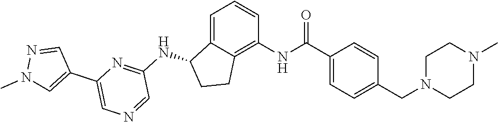 | 523.3 | ¹H NMR (62 MHz, acetone-d₆) δ 9.15 (s, 1H), 8.15 (s, 1H), 8.07 (s, 2H), 7.97 (s, 1H), 7.93 (s, 1H), 7.84 (s, 1H), 7.74-7.40 (m, 3H), 7.23 (s, 1H), 7.15 (s, 1H), 6.55 (d, J = 8.6 Hz, 1H), 5.99-5.64 (m, 1H), 3.93 (s, 3H), 3.64 (s, 2H), 3.40- 3.15 (m, 2H), 2.77-2.53 (m, 9H), 2.46 (s, 3H), 1.96-1.76 (m, 1H). |

### Example 26

### (S)-1-((6-(1-methyl-1H-pyrazol-4-yl) pyrazin-2-yl)amino)-N-(m-tolyl)-2,3-dihydro-1H-indene-4-carboxamide

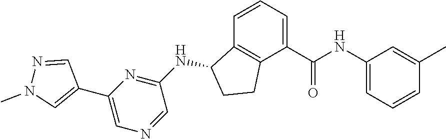

Compound 26 was prepared by the following steps:

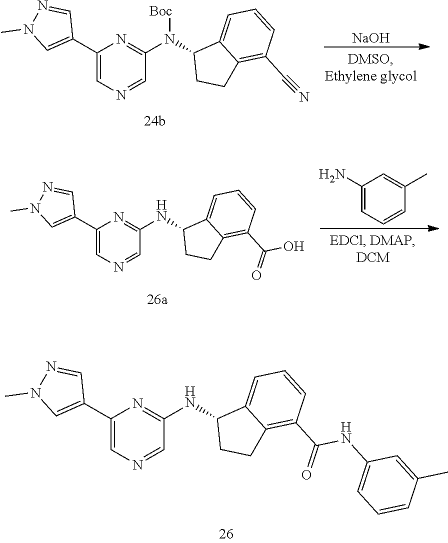

Step 1: To a solution of 24b (2082 mg, 5.0 mmol) in a mixed solvent of DMSO (8.0 mL) and glycol (2 mL) was added 50% aqueous NaOH (2 mL). The resulting mixture was heated to 100° C. and stirred for 12 h. TLC analysis showed the reaction was complete. After cooling to room temperature, conc. HCl (3 mL) was added. The resulting precipitate was collected by filtration, and dried to give compound 26a as light green solid (1459 mg, yield: 87%). ESI-MS (m/z): 336.1 [M+H]⁺.

Step 2: To a solution of 26a (34 mg, 0.10 mmol) and 3-Methylaniline (16 mg, 0.15 mmol) in CH₂Cl₂ (0.5 mL) was added EDCI (29 mg, 0.15 mmol) and DMAP (3 mg, 0.02 mmol) in turn. The resulting mixture was stirred at room temperature for 5 h. TLC analysis showed the reaction was complete. The mixture was wash with 5% aqueous NaHCO₃, the separated organic phase was purified by silica gel chromatography to give compound 26 as a white solid (32 mg, yield: 76%). ESI-MS (m/z): 425.15 [M+H]⁺. ¹H NMR (62 MHz, acetone-d₆) δ 9.28 (s, 1H), 8.14 (s, 1H), 8.08 (s, 1H), 7.96 (s, 1H), 7.83 (s, 1H), 7.78-7.51 (m, 3H), 7.46 (s, 1H), 7.37 (s, 1H), 7.23 (s, 1H), 7.18-6.92 (m, 1H), 6.54 (d, J=7.9 Hz, 1H), 6.07-5.61 (m, 1H), 3.93 (s, 3H), 3.46-3.05 (m, 2H), 2.75-2.41 (m, 1H), 2.34 (s, 3H), 1.96-1.74 (m, 1H).

### Example 27

### (S)—N-(1-methyl-1H-indazol-3-yl)-1-((6-(1-methyl-1H-pyrazol-4-yl) pyrazin-2-yl)amino)-2,3-dihydro-1H-indene-4-carboxamide

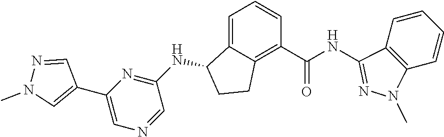 [SMILES: Cn1cc(-c2cncc(N[C@H]3CCc4c(C(=O)Nc5nn(C)c6ccccc56)cccc43)n2)cn1]

Compound 27g was prepared by the following steps:

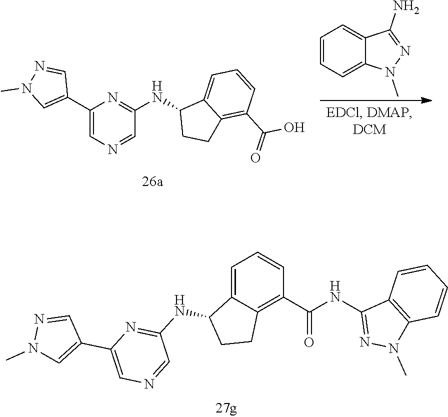 [SMILES: Cn1cc(-c2cncc(N[C@H]3CCc4c(C(=O)O)cccc43)n2)cn1 ; Cn1nc(N)c2ccccc21 ; Cn1cc(-c2cncc(N[C@H]3CCc4c(C(=O)Nc5nn(C)c6ccccc56)cccc43)n2)cn1]

To a solution of 26a (34 mg, 0.10 mmol) and 1-methyl-1H-indazol-3-amine (16 mg, 0.12 mmol) in CH₂Cl₂ (0.5 mL) was added EDCI (29 mg, 0.12 mmol) and DMAP (3 mg, 0.02 mmol) in turn. The resulting mixture was stirred at room temperature for 5 h. TLC analysis showed the reaction was complete. The mixture was wash with 5% aqueous NaHCO₃ (0.5 mL), the separated organic phase was purified by silica gel chromatography to give compound 27g as a white solid (30 mg, yield: 72%). ESI-MS (m/z): 465.2 [M+H]⁺. ¹H NMR (62 MHz, acetone-d₆) δ 9.76 (s, 1H), 8.15 (s, 1H), 8.09 (s, 1H), 8.05-7.87 (m, 2H), 7.84 (s, 1H), 7.78 (d, J=1.6 Hz, 1H), 7.73-7.49 (m, 2H), 7.48-7.36 (m, 2H), 7.35-6.95 (m, 2H), 6.60 (d, J=7.9 Hz, 1H), 5.74 (t, J=7.6 Hz, 1H), 3.96 (d, J=4.9 Hz, 6H), 3.66-3.05 (m, 2H), 2.85-2.23 (m, 1H), 1.93-1.00 (m, 1H).

The preparation of compounds 27a-27o refer to the synthesis of compound 26 in example 26 or compound 27g in example 27.

TABLE 8

| | | MS | |
|---|---|---|---|
| Cpds | Structure | [M + 1]⁺ | ¹H NMR |
| 27a | 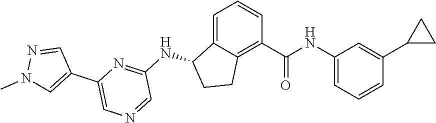 [SMILES: Cn1cc(-c2cncc(N[C@H]3CCc4c(C(=O)Nc5cccc(C6CC6)c5)cccc43)n2)cn1] | 451.2 | ¹H NMR (62 MHz, acetone-d₆) δ 9.27 (s, 1H), 8.19-7.06 (m, 10H), 6.99-6.73 (m, 1H), 6.55 (d, J = 7.9 Hz, 1H), 6.00-5.45 (m, 1H), 3.93 (s, 3H), 3.50-2.92 (m, 2H), 2.75-2.21 (m, 1H), 1.90-1.62 (m, IH), 1.22 (d, J = 9.5 Hz, 1H), 1.09-0.58 (m, 4H). |
| 27b | 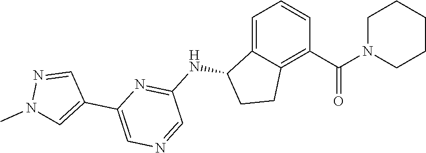 [SMILES: Cn1cc(-c2cncc(N[C@H]3CCc4c(C(=O)N5CCCCC5)cccc43)n2)cn1] | 403.2 | ¹H NMR (62 MHz, acetone-d₆) δ 8.25-7.71 (m, 4H), 7.51-7.02 (m, 3H), 6.55 (d, J = 7.9 Hz, 1H), 5.70 (q, J = 6.7, 5.8 Hz, 1H), 3.93 (s, 3H), 3.57 (s, 4H), 3.06-2.44 (m, 4H), 1.62 (s, 6H). |
| 27c | 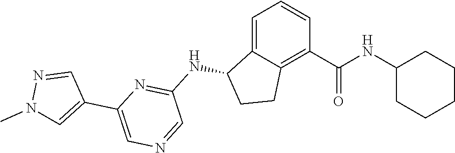 [SMILES: Cn1cc(-c2cncc(N[C@H]3CCc4c(C(=O)NC5CCCCC5)cccc43)n2)cn1] | 417.2 | ¹H NMR (62 MHz, acetone-d₆) δ 8.40-6.84 (m, 7H), 6.49 (d, J = 7.9 Hz, 1H), 5.67 (d, J = 6.7 Hz, 1H), 3.92 (s, 4H), 3.17 (s, 2H), 2.79 (s, 2H), 1.98- 0.61 (m, 11H). |
| 27d | 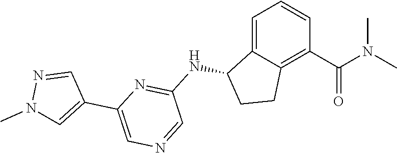 | 363.2 | ¹H NMR (62 MHz, acetone-d₆) δ 8.25-7.67 (m, 4H), 7.52-7.03 (m, 3H), 6.57 (d, J = 8.0 Hz, 1H), 5.83 (d, J = 7.5 Hz, 1H), 3.93 (s, 3H), 2.99 (dd, J = 5.5, 2.8 Hz, 8H), 2.75-2.17 (m, 1.5H), 1.93-1.61 (m, 0.5H). |
| 27e | 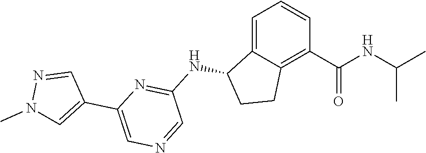 [SMILES: CC(C)NC(=O)c1cccc2c1CC[C@@H]2Nc1cncc(-c2cnn(C)c2)n1] | 377.2 | ¹H NMR (62 MHz, acetone-d₆) δ 7.89 (dd, J = 11.8, 7.1 Hz, 4H), 7.60-7.01 (m, 4H), 5.62 (dt, J = 15.4, 7.3 Hz, 2H), 4.89 (d, J = 8.2 Hz, 1H), 4.64 4.05 (m, 1H), 3.53-3.02 (m, 2H), 3.00-1.94 (m, 3H), 1.27 (d, J = 6.5 Hz, 7H). |
| 27f | 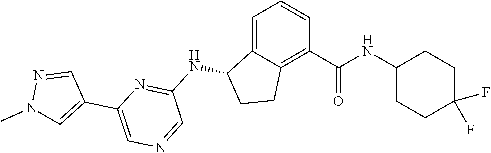 [SMILES: Cn1cc(-c2cncc(N[C@H]3CCc4c(C(=O)NC5CCC(F)(F)CC5)cccc43)n2)cn1] | 453.2 | ¹H NMR (62 MHz, acetone-d₆) δ 7.89 (dd, J = 12.0, 7.4 Hz, 4H), 7.59-7.19 (m, 3H), 6.17-5.34 (m, 2H), 4.85 (d, J = 8.2 Hz, 1H), 3.95 (s, 4H), 3.24 (q, J = 7.4, 6.0 Hz, 2H), 2.96-2.42 (m, 2H), 2.10 (t, J = 7.0 Hz, 6H), 1.69-1.10 (m, 2H). |
| 27g | 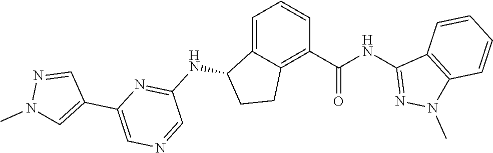 [SMILES: Cn1cc(-c2cncc(N[C@H]3CCc4c(C(=O)Nc5nn(C)c6ccccc56)cccc43)n2)cn1] | 465.2 | ¹H NMR (62 MHz, acetone-d₆) δ 9.76 (s, 1H), 8.15 (s, 1H), 8.09 (s, IH), 8.05-7.87 (m, 2H), 7.84 (s, 1H), 7.78 (d, J = 1.6 Hz, 1H), 7.73- 7.49 (m, 2H), 7.48-7.36 (m, 2H), 7.35-6.95 (m, 2H), 6.60 (d, J = 7.9 Hz, 1H), 5.74 (t, J = 7.6 Hz, 1H), 3.96 (d, J = 4.9 Hz, 6H), 3,66-3.05 (m, 2H), 2.85-2.23 (m, 1H), 1.93-1.00 (m, 1H). |
| 27h | 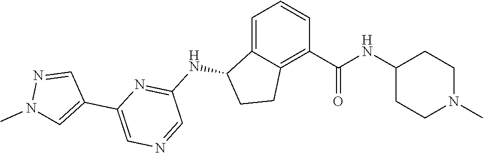 | 432.2 | ¹H NMR (62 MHz, DMSO-d₆) δ 8.28-7.99 (m, 3H), 7.95 (s, 1H), 7.76 (s, 1H), 7.59-7.02 (m, 4H), 5.71-5.34 (m, 1H), 3.87 (s, 3H), 3.26-2.63 (m, 5H), 2.22 (s, 3H), 2.09-0.90 (m, 8H). |
| 27i | 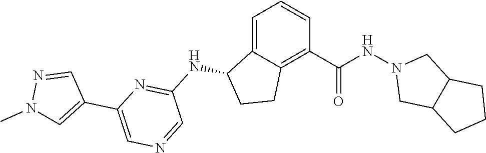 [SMILES: Cn1cc(-c2cncc(N[C@H]3CCc4c(C(=O)NN5CC6CCCC6C5)cccc43)n2)cn1] | 444.2 | ¹H NMR (62 MHz, acetone-d₆) δ 8.38-6.94 (m, 8H), 6.53 (d, J = 8.1 Hz, 1H), 5.68 (d, J = 7.2 Hz, 1H), 3.92 (s, 3H), 3.15 (s, 4H), 2.58 (d, J = 7.3 Hz, 4H), 1.44 (d, J = 17.6 Hz, 8H). |
| 27j | 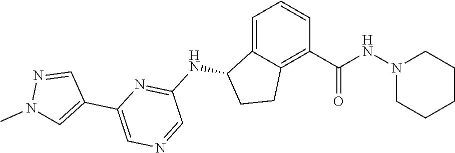 [SMILES: Cn1cc(-c2cncc(N[C@H]3CCc4c(C(=O)NN5CCCCC5)cccc43)n2)cn1] | 418.2 | ¹H NMR (62 MHz, acetone-d₆) δ 8.49-6.94 (m, 8H), 6.59 (d, J = 8.0 Hz, 1H), 5.68 (q, J = 7.8 Hz, 1H), 3.92 (s, 3H), 3.38-3.00 (m, 2H), 2.52 (s, 6H), 1.65 (d, J = 11.4 Hz, 6H). |
| 27k | 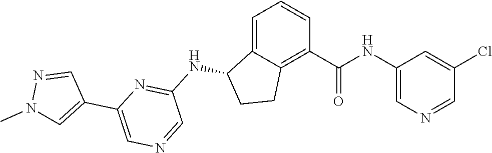 [SMILES: Cn1cc(-c2cncc(N[C@H]3CCc4c(C(=O)Nc5cncc(Cl)c5)cccc43)n2)cn1] | 446.1 | ¹H NMR (62 MHz, acetone-d₆) δ 9.78 (s, 1H), 8.85 (d, J = 2.2 Hz, 2H), 8.53 (t, J = 2.2 Hz, 1H), 8.33 (d, J = 2.2 Hz, 1H), 8.14 (s, 1H), 8.08 (s, 1H), 7.96 (s, 1H), 7.83 (s, 1H), 7.79-7.57 (m, 2H), 7.56-7.07 (m, 2H), 7.45-7.11 (m, 1H), 6.57 (d, J = 7.8 Hz, 1H), 5.71 (t, J = 11.0 Hz, 1H), 3.93 (s, 3H), 3.57- 3.00 (m, 2H), 2.75-2.22 (m, 1H), 1.88-0.72 (m, 1H). |
| 27l | 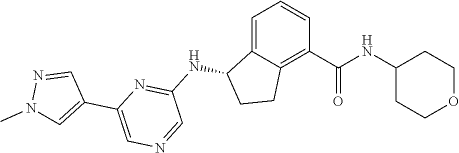 [SMILES: Cn1cc(-c2cncc(N[C@H]3CCc4c(C(=O)NC5CCOCC5)cccc43)n2)cn1] | 419.1 | ¹H NMR (400 MHz, DMSO-d₆) δ 8.21 (s, 1H), 8.12 (d, J = 7.7 Hz, 1H), 8.03 (s, 1H), 7.96 (s, 1H), 7.76 (s, 1H), 7.49-7.34 (m, 3H), 7.24 (t, J = 7.5 Hz, 1H), 5.57 (d, J = 8.4 Hz, 1H), 3.89 (s, 3H), 3.67-3.57 (m, 5H), 3.27-2.93 (m, 2H), 2.42-2.00 (m, 1H), 1.94-1.83 (m, 1H), 1.77 (d, J = 11.8 Hz, 2H), 1.64-1.49 (m, 2H). |
| 27m | 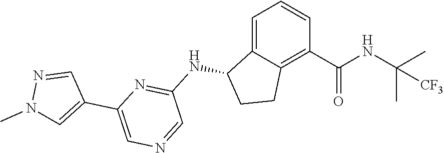 [SMILES: Cn1cc(-c2cncc(N[C@H]3CCc4c(C(=O)NC(C)(C)C(F)(F)F)cccc43)n2)cn1] | 445.2 | ¹H NMR (62 MHz, acetone-d₆) δ 8.12 (s, 1H), 8.07 (s, 1H), 7.95 (s, 1H), 7.81 (s, 1H), 7.58- 7.44 (m, 1H), 7.39 (s, 1H), 7.33-7.05 (m, 2H), 6.50 (d, J = 8.1 Hz, 1H), 5.67 (q, J = 7.7 Hz, 1H), 3.92 (s, 3H), 3.42-2.97 (m, 2H), 2.69-2.21 (m, 2H), 1.73 (s, 6H). |
| 27n | 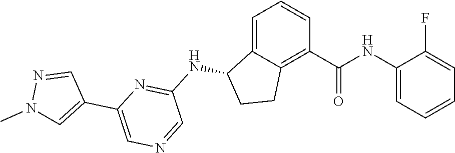 [SMILES: Cn1cc(-c2cncc(N[C@H]3CCc4c(C(=O)Nc5ccccc5F)cccc43)n2)cn1] | 429.1 | ¹H NMR (400 MHz, DMSO-d₆) δ 10.00 (s, 1H), 8.23 (s, 1H), 8.05 (s, 1H), 7.98 (s, 1H), 7.78 (s, 1H), 7.75-7.59 (m, 2H), 7.45 (t, J = 6.7 Hz, 2H), 7.37-7.16 (m, 4H), 5.70-5.53 (m, 1H), 3.88 (s, 3H), 3.29-2.97 (m, 1H), 2.75-2.59 (m, 1H), 1.98-1.86 (m, 2H). |
| 27o | 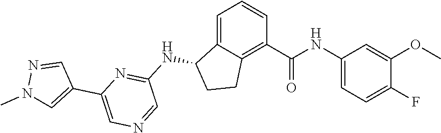 | 459.2 | ¹H NMR (62 MHz, acetone-d₆) δ 8.04 (s, 1H), 7.93 (s, 1H), 7.86 (s, 2H), 7.73 (s, 1H), 7.69-7.62 (m, 1H), 7.61-7.51 (m, 1H), 7.50-7.31 (m, 1H), 7.27 (s, 1H), 7.24-6.83 (m, 2H), 5.54 (q, J = 7.7 Hz, 1H), 4.91 (d, J = 8.2 Hz, 1H), 3.93 (s, 6H), 3.74-3.04 (m, 2H), 3.02-1.90 (m, 1H), 1.64-0.75 (m, 1H). |

### Example 28

The preparation of compounds 28a-28b refer to the synthesis of compound 12 in example 12.

TABLE 9

| | | MS | |
|---|---|---|---|
| Cpds | Structure | [M+1]⁺ | ¹H NMR |
| 28a | 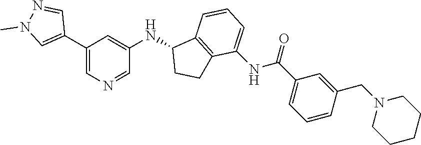 [SMILES: Cn1cc(-c2cncc(N[C@H]3CCc4c(NC(=O)c5cccc(CN6CCCCC6)c5)cccc43)c2)cn1] | 507.3 | ¹H NMR (400 MHz, DMSO-d₆) δ 10.02 (s, 1H), 8.17 (s, 1H), 8.04 (s, 1H), 7.94 (s, 1H), 7.88 (s, 3H), 7.53-7.46 (m, 2H), 7.36 (d, J = 7.7 Hz, 1H), 7.26-7.21 (m, 2H), 7.15 (d, J = 7.6 Hz, 1H), 6.27 (d, J = 8.4 Hz, 1H), 5.19-5.13 (m, 1H), 3.87 (s, 3H), 3.50 (s, 2H), 2.99-2.80 (m, 2H), 2.35 (s, 4H), 1.84-1.74 (m, 1H), 1.54-1.46 (m, 4H), 1.40 (s, 2H). |
| 28b | 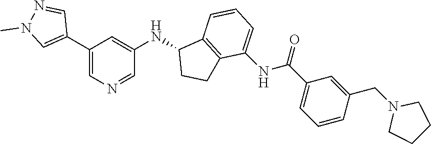 [SMILES: Cn1cc(-c2cncc(N[C@H]3CCc4c(NC(=O)c5cccc(CN6CCCC6)c5)cccc43)c2)cn1] | 493.3 | ¹H NMR (400 MHz, DMSO-d₆) δ 10.03 (s, 1H), 8.17 (s, 1H), 8.04 (s, 1H), 7.95 (s, 1H), 7.91 (s, 1H), 7.88-7.86 (m, 2H), 7.55-7.46 (m, 2H), 7.36 (d, J = 7.7 Hz, 1H), 7.26-7.21 (m, 2H), 7.15 (d, J = 7.4 Hz, 1H), 6.27 (d, J = 8.5 Hz, 1H), 5.19-5.13 (m, 1H), 3.87 (s, 3H), 3.69 (s, 2H), 2.97-2.79 (m, 2H), 2.59-2.55 (m, 2H), 1.83-1.71 (m, 5H). |

### Example 29

### methyl(S)-(1-((6-(1-methyl-1H-pyrazol-4-yl) pyrazin-2-yl)amino)-2,3-dihydro-1H-inden-4-yl) carbamate

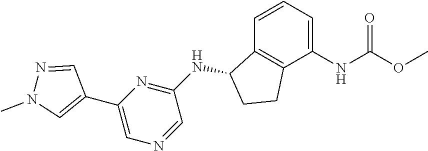

Compound 29 was prepared by the following steps:

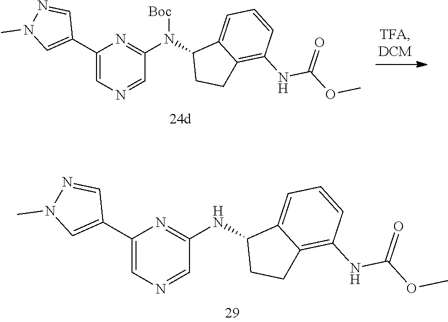

To the solution of 24d (23 mg, 0.05 mmol) in CH₂Cl₂ (0.5 mL) was added CF₃COOH (0.2 mL) and the mixture was stirred at room temperature for 3 h. TLC analysis showed the reaction was complete. 10% aqueous NaOH (2 mL) was added to the reaction mixture, and the resulting mixture was extracted with CH₂Cl₂ (5 mL*3). The combined organic layers were dried over Na₂SO₄, filtrated and concentrated. The residue was purified by silica gel chromatography to give product as a white solid (7 mg, yield: 38%). ESI-MS (m/z): 365.10 [M+H]⁺. ¹H NMR (62 MHz, acetone-d₆) δ 8.14 (s, 1H), 8.06 (s, 1H), 7.96 (s, 1H), 7.82 (s, 1H), 7.73-7.58 (m, 1H), 7.30-7.01 (m, 2H), 6.47 (d, 1H), 5.93-5.48 (m, 1H), 5.43-5.27 (m, 1H), 3.92 (s, 3H), 3.70 (s, 3H), 3.30-3.12 (m, 1H), 2.71-2.40 (m, 2H), 1.96-1.69 (m, 1H).

### Example 30

### (S)—N-(1-((6-chloropyrazin-2-yl)amino)-2,3-dihydro-1H-inden-4-yl)-3-methylbenzamide

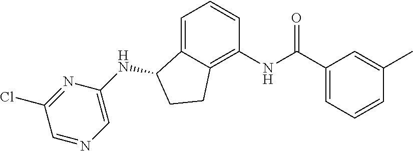 [SMILES: Cc1cccc(C(=O)Nc2cccc3c2CC[C@@H]3Nc2cncc(Cl)n2)c1]

Compound 30 was prepared by the following steps:

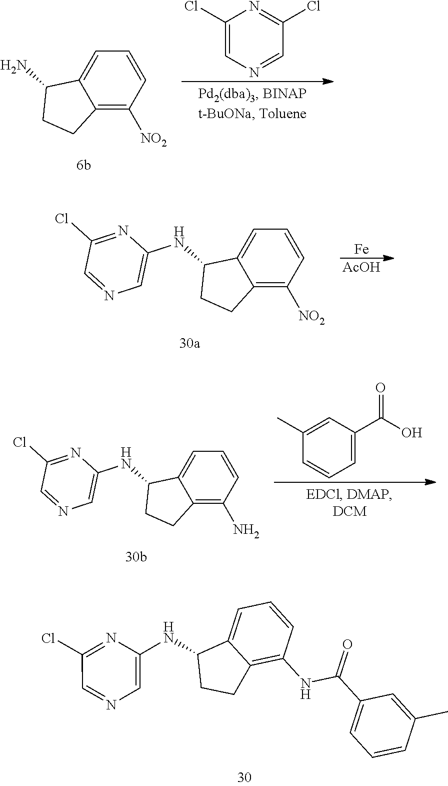

Step 1: To a solution of 2,6-Dichloropyrazine (75 mg, 0.50 mmol) and 6b (98 mg, 0.55 mmol) in toluene (1 mL) was added Pd₂(dba)₃ (46 mg, 0.05 mmol), BINAP (32 mg, 0.05 mmol) and t-BuONa (145 mg, 1.50 mmol) in turn. The resulting mixture was refluxed 5 h under N₂ atmosphere. TLC analysis showed the reaction was complete. The reaction mixture was diluted with EtOAc (8 mL) and washed with water (4 mL). The separated organic phase was purified by silica gel chromatography to give product as yellow solid (39 mg, yield: 27%). ESI-MS (m/z): 291.1 [M+H]⁺.

Step 2: To a solution of 30a (30 mg, 0.10 mmol) in CH₃COOH (1 mL) was added ferrous power (56 mg, 1.00 mmol). The mixture was reacted at 50° C. for 2 h. TLC analysis showed the reaction was complete. The reaction mixture was diluted with EtOAc (8 mL) and washed with 5% aqueous NaHCO₃ (5 mL). The separated organic phase was purified by silica gel chromatography to give product as a white solid (22 mg, yield: 85%). ESI-MS (m/z): 261.1 [M+H]⁺.

Step 3: To a solution of 30b (22 mg, 0.08 mmol) and 3-Methylbenzoic acid (16 mg, 0.12 mmol) in CH₂Cl₂ (0.5 mL) was added EDCI (23 mg, 0.12 mmol) and DMAP (3 mg, 0.02 mmol) in turn. The resulting mixture was reacted at room temperature for 5 h. TLC analysis showed the reaction was complete. The mixture was wash with 5% aqueous NaHCO₃. The separated organic phase was purified by silica gel chromatography to give product as a light yellow solid (26 mg, yield: 81%). ESI-MS (m/z): 379.00 [M+H]⁺. ¹H NMR (62 MHz, acetone-d₆) δ 9.97 (s, 1H), 8.01 (d, J=7.9 Hz, 1H), 7.96 (s, 1H), 7.80 (s, 1H), 7.77 (s, 1H), 7.75 (s, 1H), 7.45-7.36 (m, 3H), 7.23 (t, J=7.7 Hz, 1H), 7.14 (d, J=7.5 Hz, 1H), 5.50-5.37 (m, 1H), 3.07-2.91 (m, 1H), 2.90-2.74 (m, 1H), 2.59-2.52 (m, 1H), 2.41 (s, 3H), 1.94-1.76 (m, 1H).

### Example 31

### (S)-3-methyl-N-(1-((6-(4-methylpiperazin-1-yl) pyrazin-2-yl)amino)-2,3-dihydro-1H-inden-4-yl)benzamide

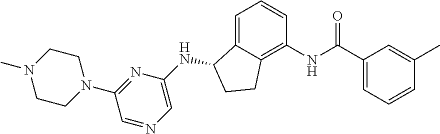

Compound 31 was prepared by the following steps:

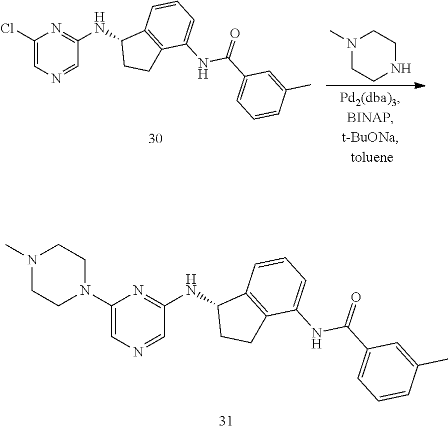

A solution of 30 (20 mg, 0.05 mmol), N-methylpiperazine (10 mg, 0.10 mmol), Pd₂ (dba)₃ (2 mg, 0.002 mmol), BINAP (6 mg, 0.01 mmol) and t-BuONa (10 mg, 0.10 mmol) in toluene (0.1 mL) was degassed with N₂ for three times, then heated to 90° C. and stirred until TLC analysis showed the reaction was complete. After natural cooling to room temperature, the mixture was concentrated to dryness, and the residue was purified by silica gel chromatography to give product (5 mg, yield: 21%). ESI-MS (m/z): 443.2 [M+H]⁺. ¹H NMR (400 MHz, DMSO-d₆) δ 9.95 (s, 1H), 7.79 (s, 1H), 7.77 (s, 1H), 7.47-7.39 (m, 2H), 7.37-7.27 (m, 3H), 7.21-7.17 (m, 1H), 7.10-7.08 (m, 2H), 5.50-7.44 (m, 1H), 3.45 (s, 4H), 2.95-2.89 (m, 1H), 2.84-2.75 (m, 1H), 2.41-2.37 (m, 8H), 2.20 (s, 3H), 1.88-1.78 (m, 1H).

### Example 32

### (S)—N-(1-((6-((2-(dimethylamino)ethyl)(methyl)amino) pyrazin-2-yl)amino)-2,3-dihydro-1H-inden-4-yl)-3-methylbenzamide

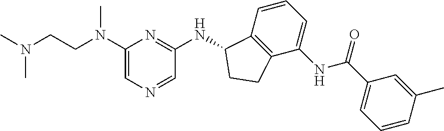

The preparation of compound 32 refers to the synthesis of compound 31 in example 31.

ESI-MS (m/z): 445.2 [M+H]⁺. ¹H NMR (62 MHz, acetone-d₆) δ 9.13 (s, 1H), 7.82 (s, 1H), 7.77-7.49 (m, 2H), 7.48-7.29 (m, 3H), 7.28-7.01 (m, 3H), 6.20 (d, 1H), 5.92-5.22 (m, 1H), 3.73 (t, J=7.1 Hz, 2H), 3.24 (s, 3H), 3.05-2.51 (m, 5H), 2.41 (s, 3H), 2.34 (s, 6H), 1.95-1.75 (m, 1H).

### Example 33

### (S)-1-((6-chloropyrazin-2-yl)amino)-N-(m-tolyl)-2,3-dihydro-1H-indene-4-carboxamide

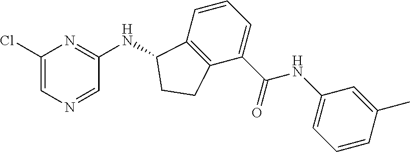 [SMILES: Cc1cccc(NC(=O)c2cccc3c2CC[C@@H]3Nc2cncc(Cl)n2)c1]

### (S)-1-((6-(1-methyl-1,2,3,6-tetrahydropyridin-4-yl) pyrazin-2-yl)amino)-N-(m-tolyl)-2,3-dihydro-1H-indene-4-carboxamide

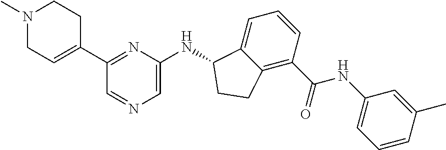

Compounds 33c and 33 were prepared by the following steps:

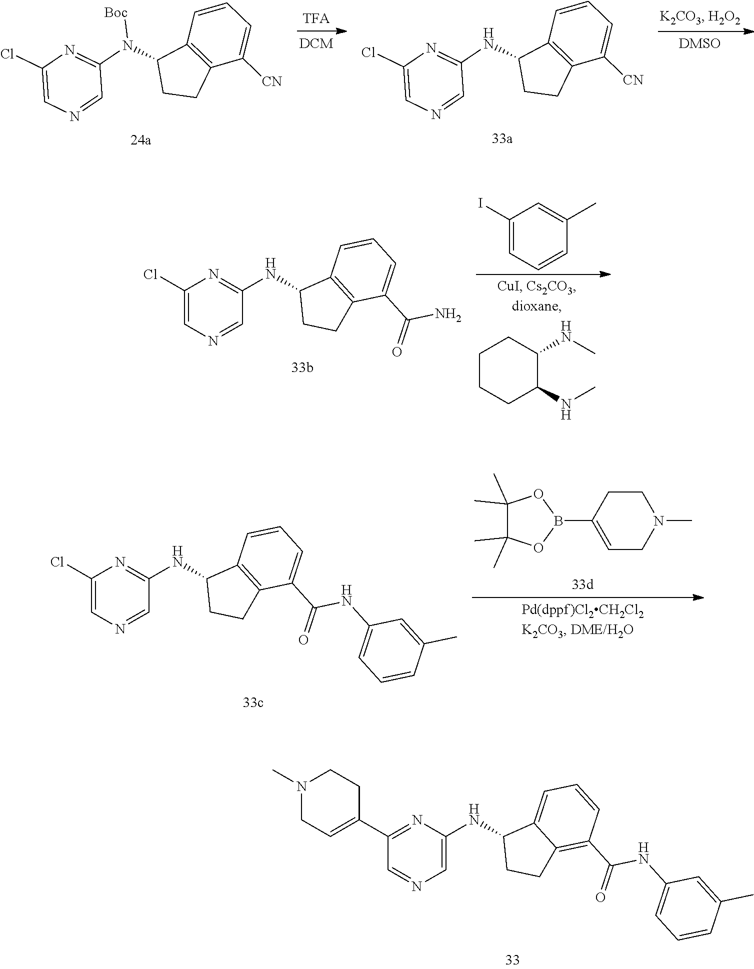

Step 1: To a solution of 24a (2.85 g, 6.9 mmol) in CH₂Cl₂ (50 mL) was added CF₃COOH (20 mL). The resulting mixture was stirred at room temperature for 3 h. TLC analysis showed the reaction was complete. The mixture was washed with 10% aqueous NaOH (50 mL), dried over Na₂SO₄, and concentrated to dryness under reduced pressure to give product (1.61 g, yield: 90%). ESI-MS (m/z): 271.1 [M+H]⁺.

Step 2: To a stirred solution of 33a (1.35 g, 5.0 mmol) in DMSO (12 mL) was added K₂CO₃ (2.07 mg, 15.0 mmol) and 30% H₂O₂ (4 mL) in turn. The resulting mixture was stirred at room temperature for 1 h. TLC analysis showed the reaction was complete. The reaction mixture was diluted with water (60 mL), and extracted with EtOAc (60 mL*3). The combined organic phases were concentrated to dryness, and the residue was purified by silica gel chromatography to give product (878 mg, yield: 61%). ESI-MS (m/z): 289.1 [M+H]⁺.

Step 3: A mixture of 30b (870 mg, 3.0 mmol), 3-Iodobenzylamine (1.30 g, 6.0 mmol), Cs₂CO₃ (1.95 g, 6.0 mmol), CuI (58 mg, 0.3 mmol) and (1S, 2S)-(+)-N,N′-Dimethylcyclohexanediamine (85 mg, 0.6 mmol) in dioxane (3 mL) was heated to 80° C. and stirred under N₂ until TLC analysis showed the reaction was complete. The reaction mixture was concentrated to dryness and the residue was purified by silica gel chromatography to give product (682 mg, yield: 60%). ESI-MS (m/z): 379.1 [M+H]⁺.

Step 4: A solution of 33c (50 mg, 0.13 mmol), 33d (39 mg, 0.17 mmol), Pd(dppf)Cl₂·CH₂Cl₂ (1 mg, 0.001 mmol) and K₃PO₄ (85 mg, 0.40 mmol) in mixed solvent of DME (0.6 mL) and water (0.3 mL) was degassed with N₂ for three times, then heated to 90° C. and stirred until TLC analysis showed the reaction was complete. After natural cooling to room temperature, the reaction mixture was concentrated to dryness and the residue was purified by silica gel chromatography to give product 33 (31 mg, yield: 53%). ESI-MS (m/z): 440.1 [M+H]⁺. ¹H NMR (62 MHz, acetone-d₆) δ 9.24 (s, 1H), 7.69 (s, 1H), 7.58 (s, 1H), 7.54 (s, 1H), 7.40 (s, 2H), 7.35 (s, 2H), 7.24 (s, 1H), 7.16-6.81 (m, 1H), 6.20 (d, J=7.9 Hz, 1H), 5.82-5.31 (m, 1H), 3.71-3.40 (m, 4H), 3.39-3.02 (m, 2H), 2.80-2.57 (m, 1H), 2.56-2.36 (m, 4H), 2.33 (s, 3H), 2.24 (s, 3H), 1.96-1.79 (m, 1H).

### Example 34

### (S)-1-((6-(4-methylpiperazin-1-yl) pyrazin-2-yl)amino)-N-(m-tolyl)-2,3-dihydro-1H-indene-4-carboxamide

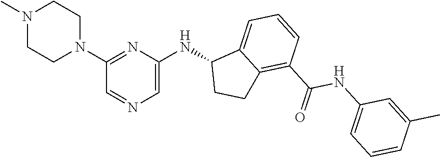

The preparation of compound 34 refers to the synthesis of compound 31 in example 31.

ESI-MS (m/z): 443.1 [M+H]⁺. ¹H NMR (62 MHz, acetone-d₆) δ 9.24 (s, 1H), 7.69 (s, 1H), 7.58 (s, 1H), 7.54 (s, 1H), 7.40 (s, 2H), 7.35 (s, 2H), 7.24 (s, 1H), 7.16-6.81 (m, 1H), 6.20 (d, J=7.9 Hz, 1H), 5.82-5.31 (m, 1H), 3.71-3.40 (m, 4H), 3.39-3.02 (m, 2H), 2.80-2.57 (m, 1H), 2.56-2.36 (m, 4H), 2.33 (s, 3H), 2.24 (s, 3H), 1.96-1.79 (m, 1H).

### Example 35

### (S)—N⁴-(4-isopropylthiazol-2-yl)-N¹-(6-(1-methyl-1H-pyrazol-4-yl) pyrazin-2-yl)-2,3-dihydro-1H-indene-1,4-diamine

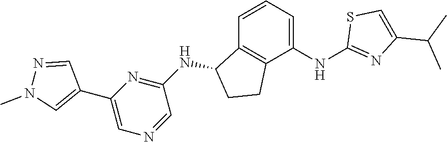 [SMILES: CC(C)c1csc(Nc2cccc3c2CC[C@@H]3Nc2cncc(-c3cnn(C)c3)n2)n1]

Compound 35 was prepared by the following steps:

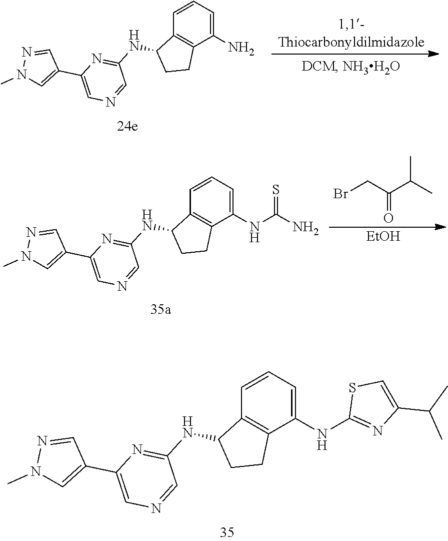

Step 1: To a solution of 24e (153 mg, 0.5 mmol) in CH₂Cl₂ (0.6 mL) was added N,N′-Thiocarbonylbisimidazole (107 mg, 0.6 mmol). The mixture was stirred at room temperature for 1 h, then Ammonium hydroxide (25%, 68 mg) was added to the above mixture and the resulting mixture was stirred for another 1 h. Water (30 mL) was added to the reaction mixture, and the mixture was extracted with CH₂Cl₂ (30 mL*3). The combined organic phases were washed with brine (30 mL), dried over Na₂SO₄, filtrated, and concentrated to dryness to give product as a oil (180 mg, yield: 98.5%). ESI-MS (m/z): 366.1 [M+H]⁺.

Step 2: The solution of 35a (73 mg, 0.2 mmol) and 1-Bromo-3-methyl-2-butanone (36 mg, 0.22 mmol) in EtOH (2 mL) was heated to 80° C. and stirred for 2 h. The resulting mixture was concentrated, and the residue was purified by silica gel chromatography to give product 35 as a white solid (50 mg, yield: 57.9%). ESI-MS (m/z): 432.2 [M+H]⁺. ¹H NMR (62 MHz, acetone-d₆) δ 8.54 (s, 1H), 8.29-7.73 (m, 5H), 7.38-6.89 (m, 2H), 6.67-6.25 (m, 2H), 5.94-5.66 (m, 1H), 3.92 (s, 3H), 3.21-1.75 (m, 5H), 1.26 (d, J=6.8 Hz, 6H).

### Example 36

The preparation of compounds 36a-36c refer to the synthesis of compound 35 in example 35.

TABLE 10

| | | MS | |
|---|---|---|---|
| Cpds | Structure | [M + 1]⁺ | ¹H NMR |
| 36a | 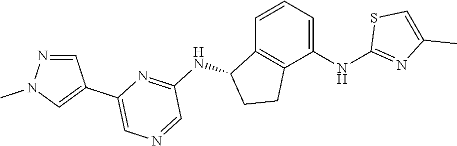 | 404.2 | ¹H NMR (62 MHz, acetone-d₆) δ 8.52 (s, 1H), 8.26-8.02 (m, 3H), 7.97 (s, 1H), 7.83 (s, 1H), 7.38-6.87 (m, 2H), 6.68 -6.26 (m, 2H), 5.95-5.46 (m, 1H), 3.92 (s, 3H), 3.21-1.61 (m, 7H). |
| 36b | 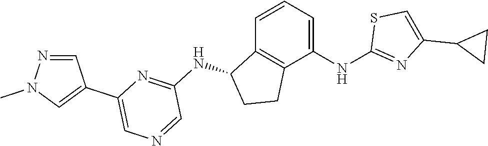 [SMILES: Cn1cc(-c2cncc(N[C@H]3CCc4c(Nc5nc(C6CC6)cs5)cccc43)n2)cn1] | 430.2 | ¹H NMR (62 MHz, acetone-d₆) δ 8.24-7.70 (m, 5H), 7.39-6.86 (m, 2H), 6.37 (s, 1H), 5.95-5.65 (m, 1H), 3.92 (s, 3H), 3.21-1.61 (m, 5H), 0.94-0.69 (m, 4H). |
| 36c | 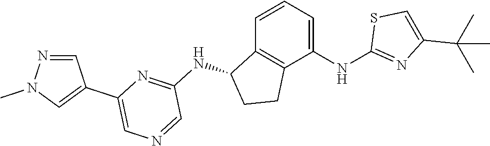 [SMILES: Cn1cc(-c2cncc(N[C@H]3CCc4c(Nc5nc(C(C)(C)C)cs5)cccc43)n2)cn1] | 446.2 | ¹H NMR (62 MHz, acetone-d₆) δ 8.56 (s, 1H), 8.32-8.02 (m, 3H), 7.97 (s, 1H), 7.83 (s, 1H), 7.50-6.78 (m, 2H), 6.36 (s, 2H), 6.05-5.46 (m, 1H), 3.92 (s, 3H), 3.31-1.50 (m, 4H), 1.31 (s, 9H). |

### Example 37

### (S)—N⁴-(5-isopropylthiazol-2-yl)-N¹-(6-(1-methyl-1H-pyrazol-4-yl) pyrazin-2-yl)-2,3-dihydro-1H-indene-1,4-diamine

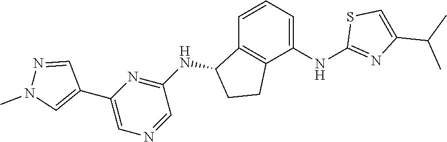 [SMILES: CC(C)c1csc(Nc2cccc3c2CC[C@@H]3Nc2cncc(-c3cnn(C)c3)n2)n1]

Compound 37 was prepared by the following steps:

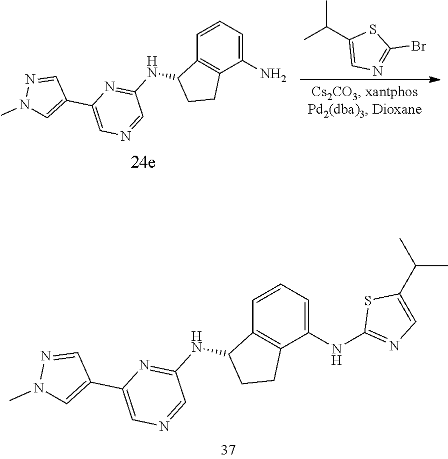

To a reaction flask containing dioxane (0.5 mL) was added 24e (31 mg, 0.1 mmol), Cs₂CO₃ (82 mg, 0.25 mmol), 5-Isopropyl-2-bromothiazole (31 mg, 0.15 mmol), Xantphos (6 mg, 0.01 mmol) and Pd₂(dba)₃ (9 mg, 0.01 mmol) in turn. The resulting mixture was degassed under vacuum and purged with N₂ then heated to 100° C. and stirred for 3 h. Water (20 mL) was added to the reaction mixture and the mixture was extracted with EtOAc (15 mL*3). The combined organic phases were washed with brine (20 mL), dried over Na₂SO₄, filtrated, and concentrated. The residue was purified by silica gel chromatography to give product 37 as white solid (10 mg, yield: 23%). ESI-MS (m/z): 432.2 [M+H]⁺. ¹H NMR (62 MHz, DMSO-d₆) δ 9.25 (s, 1H), 8.21 (s, 1H), 8.09-7.83 (m, 3H), 7.74 (s, 1H), 7.47-6.77 (m, 4H), 5.82-5.34 (m, 1H), 3.86 (s, 3H), 3.17-1.62 (m, 5H), 1.29 (s, 3H), 1.18 (s, 3H).

### Example 38

### (S)—N⁴-(1-(tert-butyl)-1H-pyrazol-3-yl)-N¹-(6-(1-methyl-1H-pyrazol-4-yl) pyrazin-2-yl)-2,3-dihydro-1H-indene-1,4-diamine

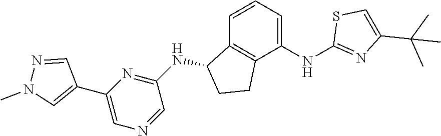 [SMILES: Cn1cc(-c2cncc(N[C@H]3CCc4c(Nc5nc(C(C)(C)C)cs5)cccc43)n2)cn1]

Compound 38 was prepared by the following steps:

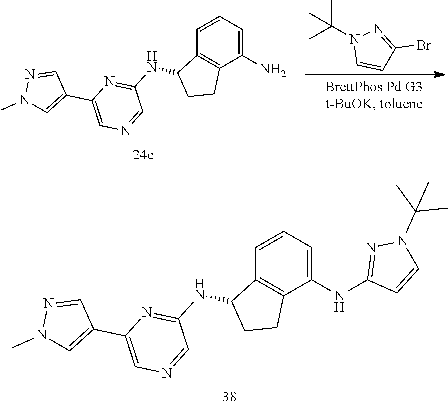

To a reaction flask containing toluene (0.5 mL) was added 24e (31 mg, 0.1 mmol), t-BuOK (44 mg, 0.39 mmol), 3-Bromo-1-tert-butylpyrazole (31 mg, 0.32 mmol) and BrettPhos Pd (II) (12 mg, 0.013 mmol) in turn. The resulting mixture was degassed under vacuum and purged with N₂, then heated to 100° C. and stirred for 4 h. Water (30 mL) was added to the reaction mixture and the mixture was extracted with EtOAc (15 mL*3). The combined organic phases were washed with brine (20 mL), dried over Na₂SO₄, filtrated, and concentrated. The residue was purified by silica gel chromatography to give product 38 as white solid (45 mg, yield: 75%). ESI-MS (m/z): 429.3 [M+H]⁺. ¹H NMR (62 MHz, Acetone-d₆) δ 8.14 (s, 1H), 8.05 (s, 1H), 7.97 (s, 1H), 7.90-7.62 (m, 2H), 7.55 (d, J=2.4 Hz, 1H), 7.29-6.63 (m, 3H), 6.54-6.22 (m, 1H), 5.89 (d, J=2.4 Hz, 1H), 5.79-5.39 (m, 1H), 3.92 (s, 3H), 3.17-1.73 (m, 4H), 1.56 (s, 9H).

### Example 39

The preparation of compounds 39a and 39b refer to the synthesis of compound 38 in example 38.

TABLE 11

| | | MS | |
|---|---|---|---|
| Cpds | Structure | [M + 1]⁺ | ¹H NMR |
| 39a | 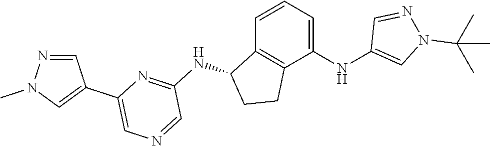 [SMILES: Cn1cc(-c2cncc(N[C@H]3CCc4c(Nc5cnn(C(C)(C)C)c5)cccc43)n2)cn1] | 429.3 | ¹H NMR (62 MHz, DMSO-d₆) δ 8.21 (s, 1H), 8.06-7.86 (m, 2H), 7.82-7.61 (m, 2H), 7.44-7.06 (m, 2H), 7.05-6.77 (m, 2H), 6.76-6.48 (m, 2H), 5.79-5.27 (m, 1H), 3.87 (s, 3H), 3.17-1.76 (m, 4H), 1.52 (s, 9H). |
| 39b | 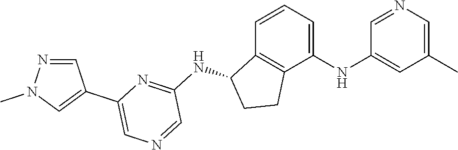 | 398.2 | ¹H NMR (62 MHz, acetone-d₆) δ 8.23 (s, 1H), 8.13-8.09 (m, 1H), 8.05-7.92 (m, 2H), 7.87-7.84 (m, 1H), 7.78 (s, 2H), 7.36 (d, J = 8.0 Hz, 1H), 7.21-6.78 (m, 4H), 5.75-5.54 (m, 1H), 3.88 (s, 3H), 2.90-2.73 (m, 2H), 2.23 (s, 3H), 2.11- 1.05 (m, 2H). |

### Example 40

### (S)—N⁴-(5-(ter-butyl)-4-methylisoxazol-3-yl)-N¹-(5-chloropyridin-3-yl)-2,3-dihydro-1H-indene-1,4-diamine

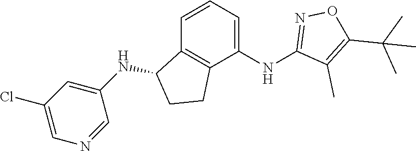 [SMILES: Cc1c(Nc2cccc3c2CC[C@@H]3Nc2cncc(Cl)c2)noc1C(C)(C)C]

### (S)—N⁴-(5-(tert-butyl)-4-methylisoxazol-3-yl)-N¹-(5-(1-methyl-1H-pyrazol-4-yl) pyridin-3-yl)-2,3-dihydro-18-indene-1,4-diamine

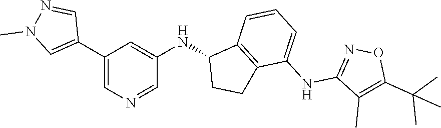 [SMILES: Cc1c(Nc2cccc3c2CC[C@@H]3Nc2cncc(-c3cnn(C)c3)c2)noc1C(C)(C)C]

Compound 40c and compound 40 were prepared by the following steps:

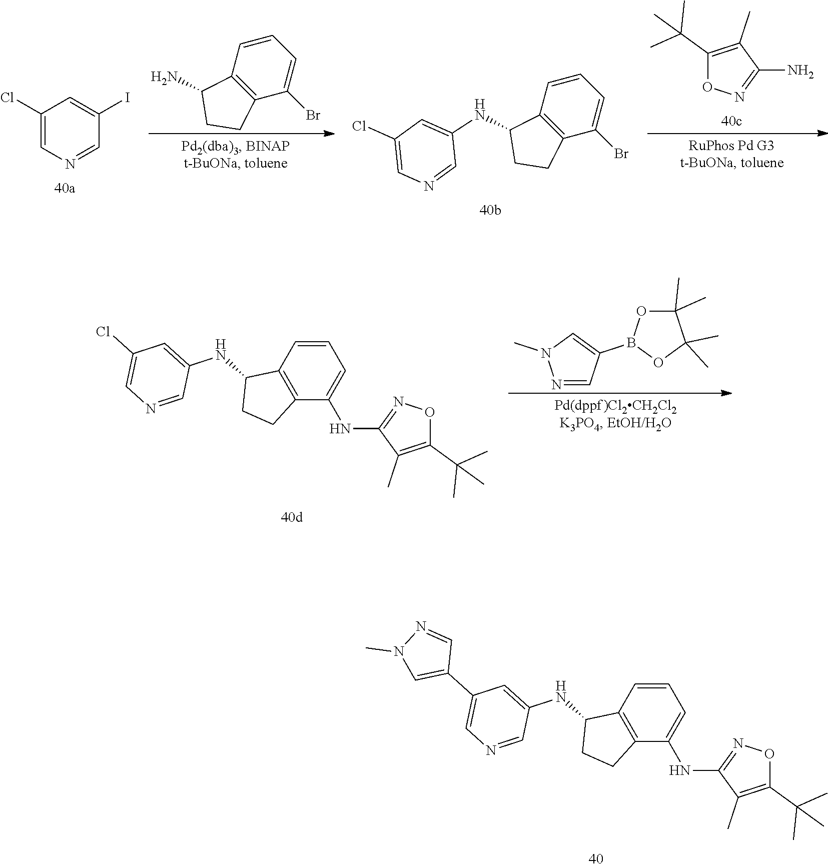

Step 1: The solution of 40a (1.35 g, 5.66 mmol), (1S)-4-Bromo-2,3-dihydro-1H-inden-1-amine (1.00 g, 4.7 mmol), t-BuONa (1.63 g, 16.98 mmol), BINAP (146.5 mg, 0.235 mmol) and Pd₂(dba)₃ (215 mg, 0.235 mmol) in toluene (10 mL) was refluxed at 100° C. for 3 h under N₂ atmosphere. TLC analysis showed the reaction was complete. EtOAc (100 mL) was added to the reaction mixture, and the mixture was washed with water (50 mL*2), dried over Na₂SO₄, and concentrated. The residue was purified by silica gel chromatography to give product 40b as a white solid (530 mg, yield: 35.5%). ESI-MS (m/z): 323.0 [M+H]⁺.

Step 2: The mixture of 40b (162 mg, 0.5 mmol), 40c (84.7 mg, 0.55 mmol), RuPhos Pd G3 (41.8 mg, 0.05 mmol) and 1-BuONa (144 mg, 1.5 mmol) in toluene (0.5 mL) was refluxed at 100° C. for 3 h under N₂ atmosphere. TLC analysis showed the reaction was complete. EtOAc (100 mL) was added to the reaction mixture, And the mixture was washed with water (50 mL*2), dried over Na₂SO₄, and concentrated. The residue was purified by silica gel chromatography to give product 40d as a white solid (100 mg, yield: 55.5%). ESI-MS (m/z): 397.0 [M+H]⁺. ¹H NMR (62 MHz, DMSO-d₆) δ 7.97 (s, 1H), 7.78 (d, J=2.4 Hz, 1H), 7.50 (d, J=2.1 Hz, 1H), 7.05-6.72 (m, 2H), 6.45 (m, 3H), 4.83 (m, 1H), 2.54 (m, 2H), 2.20 (s, 1H), 1.67 (s, 4H), 1.07 (s, 9H).

Step 3: The solution of 40d (100 mg, 0.25 mmol), 1g (72.8 mg, 0.35 mmol). Pd(dppf)Cl₂·CH₂Cl₂ (20.4 mg, 0.025 mmol) and K₃PO₄ (159 mg, 0.75 mmol) in mixed solvent of EtOH (0.4 mL) and water (0.2 mL) was refluxed at 90° C. for 6 h under N₂ atmosphere. TLC analysis showed the reaction was complete, then EtOAc (50 mL) was added to the reaction mixture. The resulting mixture was washed with water (50 mL*2), dried over Na₂SO₄, and concentrated. The residue was purified by silica gel chromatography to give product 40 as a light yellow solid (20 mg, yield: 55.5%). ESI-MS (m/z): 443.0 [M+H]⁺. ¹H NMR (62 MHz, acetone-d₆) δ 8.05 (d, J=5.5 Hz, 3H), 7.80 (s, 1H), 7.42-6.67 (m, 5H), 5.22 (s, 1H) 3.92 (s, 3H), 3.00 (s, 2H), 2.51 (s, 1H). 2.12 (s, 3H), 1.84 (s, 2H), 1.35 (s, 9H).

### Example 41

### (S)—N⁴-(5-(tert-butyl) isoxazol-3-yl)-N¹-(5-(1-methyl-1H-pyrazol-4-yl) pyridin-3-yl)-2,3-dihydro-1H-indene-1,4-diamine

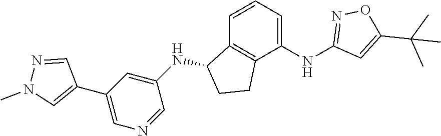 [SMILES: Cn1cc(-c2cncc(N[C@H]3CCc4c(Nc5cc(C(C)(C)C)on5)cccc43)c2)cn1]

Compound 41 was prepared by the following steps:

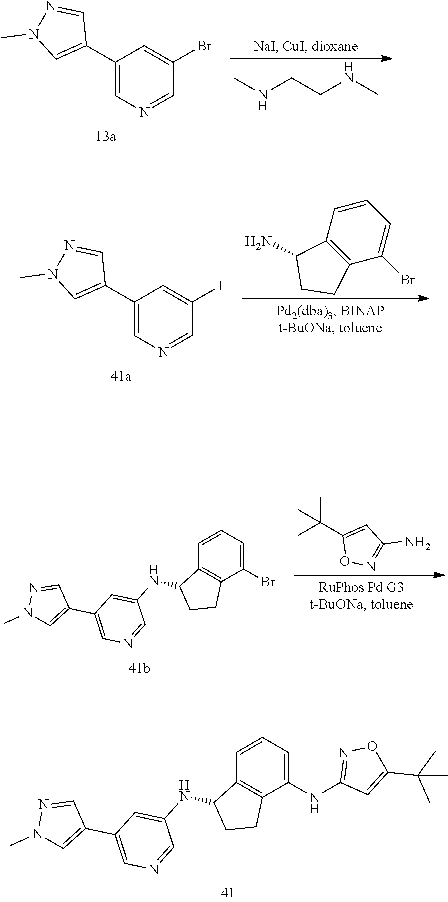

Step 1: To a solution of 13a (1.19 g, 5.0 mmol) and NaI (1.49 g, 10.0 mmol) in dioxane (5 mL) was added CuI (192 mg, 1.0 mmol) and N,N′-Dimethylethylenediamine (232 mg, 2.0 mmol). The resulting mixture was degassed with N₂ for three times, then heated to 110° C. and stirred for 24 h. After natural cooling to room temperature, CH₂Cl₂ (50 mL) was added to the reaction mixture. The resulting mixture was washed with 12% Ammonium hydroxide (50 mL) and water (50 mL), and dried over Na₂SO₄. After concentration to dryness, the crude product was used directly for the next step without further purification (1.27 g, purity: 100%, yield: 89%). ESI-MS (m/z): 286.1 [M+H]⁺.

Step 2: To a solution of 41a (850 mg, 3.0 mmol) and (1S)-4-Bromo-2,3-dihydro-1H-inden-1-amine (690 mg, 3.0 mmol), Pd₂(dba); (137 mg, 0.15 mmol) and BINAP (373 mg, 0.6 mmol) in toluene (3.0 mL) was added t-BuONa (864 mg, 9.0 mmol). The resulting mixture was degassed with N₂ for three times, then heated to 90° C. and stirred until TLC analysis showed the reaction was complete. After natural cooling to room temperature, EtOAc (30 mL) was added to the reaction mixture. The resulting mixture was washed with water (30 mL), dried over Na₂SO₄ and concentrated to dryness. The residue was purified by chromatography to give product (221 mg, yield: 19%). ESI-MS (m/z): 387.2 [M+H]⁺.

Step 3: To a reaction flask containing toluene (0.5 mL) was added 41b (37 mg, 0.1 mmol), t-BuONa (29 mg, 0.3 mmol), 3-Amino-5-tert-butylisoxazole (21 mg, 0.15 mmol) and RuPhos Pd G3 (8 mg, 0.01 mmol) in turn. The resulting mixture was degassed under vacuum and purged with N₂, then heated to 100° C. and stirred for 3 h. Water (20 mL) was added to the reaction mixture, and the resulting mixture was extracted with EtOAc (20 mL*3). The combined organic phases were washed with brine (20 mL), dried over Na₂SO₄, filtered and concentrated. The residue was purified by silica gel chromatography to give product as a white solid (30 mg, yield: 70%). ESI-MS (m/z): 429.2 [M+H]⁺. ¹H NMR (400 MHz, CDCl₃) δ 8.14-8.10 (m, 1H), 7.99-7.94 (m, 1H), 7.76-7.72 (m, 1H), 7.63 (s, 1H), 7.61-7.57 (m, 1H), 7.31-7.22 (m, 1H), 7.05-7.00 (m, 2H), 6.04 (s, 1H), 5.79 (s, 1H), 5.16-5.03 (m, 1H), 4.07-4.01 (m, 1H), 3.95 (s, 3H), 3.02-1.89 (m, 4H), 1.34 (s, 9H).

### Example 42

The preparation of compounds 42a-42t refer to the synthesis of compound 40d and 40 in example 40 or compound 41 in example 41.

TABLE 12

| | | MS | |
|---|---|---|---|
| | | [M + | |
| Cpds | Structure | 1]⁺ | ¹H NMR |
| 42a | 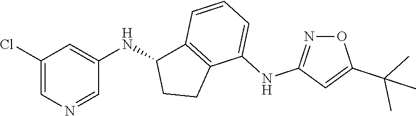 [SMILES: CC(C)(C)c1cc(Nc2cccc3c2CC[C@@H]3Nc2cncc(Cl)c2)no1] | 383.2 | ¹H NMR (62 MHz, CDCl₃) δ 8.06-7.83 (m, 2H), 7.78-7.11 (m, 2H), 7.09 6.85 (m, 2H), 6.03 (s, 1H), 5.79 (s, 1H), 5.21-4.80 (m, 1H), 4.41-3.91 (m, 1H), 3.11-1.71 (m, 4H), 1.35 (s, 9H). |
| 42b | 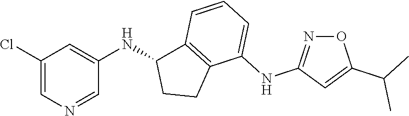 [SMILES: CC(C)c1cc(Nc2cccc3c2CC[C@@H]3Nc2cncc(Cl)c2)no1] | 369.1 | ¹H NMR (62 MHz, DMSO-d₆) δ 8.07 (s, 1H), 7.92-7.41 (m, 3H), 7.12-6.70 (m, 2H), 6.43 (s, 1H), 5.79 (s, 1H), 4.83 (m, 1H), 2.91-2.41 (m, 4H), 1.71 (m, 2H), 1.03 (m, 6H). |
| 42c | 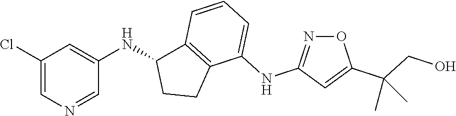 [SMILES: CC(C)(CO)c1cc(Nc2cccc3c2CC[C@@H]3Nc2cncc(Cl)c2)no1] | 399.2 | ¹H NMR (400 MHz, DMSO-d₆) δ 8.27 (s, 1H), 8.03 (m, 1H), 7.83-7.68 (m, 2H), 7.24- 7.09 (m, 2H), 6.84 (d, J = 7.4 Hz, 1H), 6.59 (d, J = 8.3 Hz, 1H), 6.02 (s, 1H), 5.12-4.99 (m, 1H), 4.99-4.91 (m, 1H), 3.45 (m, 2H), 3.08-2.92 (m, 1H), 2.85-2.72 (m, 1H), 2.59-2.52 (m, 1H), 1.88-1.71 (m, 1H), 1.22 (s, 6H). |
| 42d | 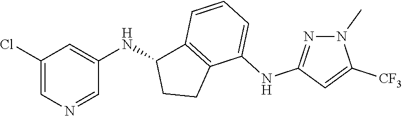 [SMILES: Cn1nc(Nc2cccc3c2CC[C@@H]3Nc2cncc(Cl)c2)cc1C(F)(F)F] | 408.1 | ¹H NMR (62 MHz, acetone-d₆) δ 8.15-8.05 (m, 1H), 7.99-7.59 (m, 2H), 7.42-6.72 (m, 4H), 6.39 (s, 1H), 5.80 (s, 1H), 5.07 (s, 1H), 3.91 (d, J = 0.9 Hz, 3H), 3.10- 2.48 (m, 2H), 2.15-1.56 (m, 2H). |
| 42e | 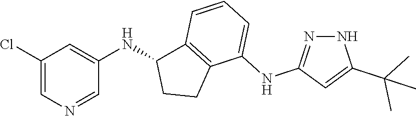 [SMILES: CC(C)(C)c1cc(Nc2cccc3c2CC[C@@H]3Nc2cncc(Cl)c2)n[nH]1] | 382.2 | ¹H NMR (62 MHz, acetone-d₆) δ 8.25-8.15 (m, 1H), 7.88-7.53 (m, 2H), 7.26-6.54 (m, 4H), 5.76 (s, 2H), 5.23-5.18 (m, 1H), 2.70- 2.65 (m, 4H), 1.81-1.75 (m, 1H), 1.25 (s, 9H). |
| 42f | 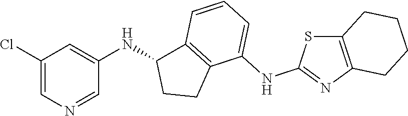 [SMILES: Clc1cncc(N[C@H]2CCc3c(Nc4nc5c(s4)CCCC5)cccc32)c1] | 397.1 | ¹H NMR (62 MHz, acetone-d₆) δ 7.32-7.01 (m, 2H), 6.85 (d, J = 2.1 Hz, 1H), 6.62 5.62 (m, 4H), 4.16 (dd, J = 7.9, 4.1 Hz 1H), 2.30-1.74 (m, 4H), 1.54-1.29 (m, 1H), 1.26-1.08 (m, 1H), 0.97-0.75 (m, 5H). |
| 42g | 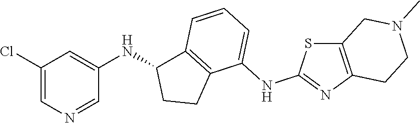 [SMILES: CN1CCc2nc(Nc3cccc4c3CC[C@@H]4Nc3cncc(Cl)c3)sc2C1] | 412.1 | ¹H NMR (62 MHz, acetone-d₆) δ 8.28-8.01 (m, 2H), 7.80 (d, J = 2.1 Hz, 1H), 7.42-6.85 (m, 3H), 5.06 (d, J = 5.2 Hz, 1H), 3.45 (S, 2H), 2.98 (dd, J = 10.5, 4.7 Hz, 5H), 2.58 (s, 1H), 2.41 (s, 3H), 1.83 (dd, J = 7.1, 4.5 Hz, 1H). |
| 42h | 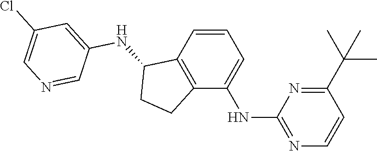 [SMILES: CC(C)(C)c1ccnc(Nc2cccc3c2CC[C@@H]3Nc2cncc(Cl)c2)n1] | 394.2 | ¹H NMR (62 MHz, acetone-d₆) δ 8.05 (dd, J = 6.6, 1.7 Hz, 2H), 7.80 (d, J = 2.1 Hz, 1H), 7.32 (d, J = 7.5 Hz, 1H), 7.19 (d, J = 2.3 Hz, 1H), 7.16-6.93 (m, 2H), 5.16 (dd, J = 9.4, 5.6 Hz, 1H), 3.21-2.93 (m, 2H), 2.82-2.36 (m, 2H), 2.11 (d, J = 2.2 Hz, 1H), 1.84 (dd, J = 7.0, 4.8 Hz, 1H), 1.40 (s, 9H). |
| 42i | 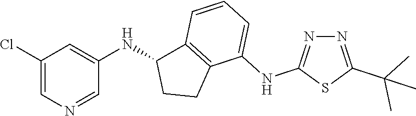 [SMILES: CC(C)(C)c1nnc(Nc2cccc3c2CC[C@@H]3Nc2cncc(Cl)c2)s1] | 400.1 | ¹H NMR (62 MHz, acetone-d₆) δ 8.43-8.09 (m, 3H), 7.83 (d, J = 2.1Hz, 1H), 7.32 (d, J = 7.9 Hz, 1H), 7.20-7.01 (m, 1H), 6.89 (d, J = 5.2 Hz, 1H), 5.83 (d, J = 8.1 Hz, 1H), 5.20 (t, J = 7.4 Hz, 1H), 3.39-3.00 (m, 2H), 2.79- 2.42 (m, 1H), 1.83 (d, J = 6.8 Hz, 1H), 1.35 (s, 9H). |
| 42j | 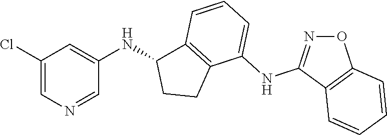 [SMILES: Clc1cncc(N[C@H]2CCc3c(Nc4noc5ccccc45)cccc32)c1] | 377.1 | ¹HNMR (400 MHz, DMSO-d₆) δ 8.70 (s, 1H), 8.21-8.03 (m, 2H), 7.83-7.72 (m, 2H), 7.68-7.55 (m, 2H), 7.37 (ddd, J = 8.0, 6.5, 1.4 Hz, 1H), 7.33-7.12 (m, 2H), 7.01 (d, J = 7.4 Hz, 1H), 6.65 (d, J = 8.3 Hz, 1H), 5.10 (q, J = 7.4 Hz, 1H), 3.09 (ddd, J = 16.1, 8.7, 3.8 Hz, 1H), 2.92 (dt, J = 16.0, 7.9 Hz, 1H), 2.59 (ddd, J = 11.8, 7.8, 3.9 Hz 1H), 1.80 (dq, J = 11.8, 7.8 Hz, 1H). |
| 42k | 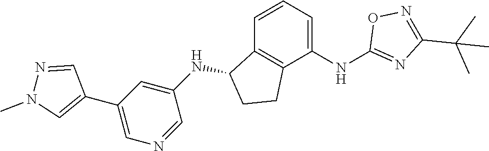 [SMILES: Cn1cc(-c2cncc(N[C@H]3CCc4c(Nc5nc(C(C)(C)C)no5)cccc43)c2)cn1] | 430.2 | 1H NMR (62 MHz, DMSO-d₆) δ 10.26 (s, 1H), 8.15 (s, 1H), 8.08-7.98 (m, 1H), 7.97- 7.80 (m, 2H), 7.75-7.48 (m, 1H), 7.41-6.94 (m, 3H), 6.39 -6.07 (m, 1H), 5.38-4.87 (m, 1H), 3.86 (s, 3H), 3.13-1.46 (m, 4H), 1.29 (s, 9H). |
| 42l | 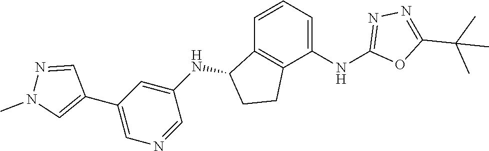 [SMILES: Cn1cc(-c2cncc(N[C@H]3CCc4c(Nc5nnc(C(C)(C)C)o5)cccc43)c2)cn1] | 430.2 | ¹H NMR (400 MHz, DMSO-d₆) δ 9.70 (s, 1H), 8.16 (s, 1H), 8.03 (s, 1H), 7.95-7.90 (m, 1H), 7.88-7.85 (m, 1H), 7.79 (d, J = 8.0 Hz, 1H), 7.27-7.17 (m, 2H), 6.99 (d, J = 7.4 Hz, 1H), 6.22 (d, J = 8.6 Hz, 1H), 5.16-5.07 (m, 1H), 3.87 (s, 3H), 3.08-1.73 (m, 4H), 1.36 (s, 9H). |
| 42m | 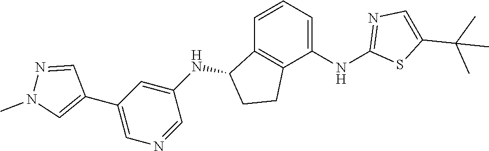 [SMILES: Cn1cc(-c2cncc(N[C@H]3CCc4c(Nc5ncc(C(C)(C)C)s5)cccc43)c2)cn1] | 445.2 | ¹H NMR (400 MHz, DMSO-d₆) δ 9.27 (s, 1H), 8.16 (s, 1H), 8.03 (d, J = 1.8 Hz, 1H), 7.95 (d, J = 8.1 Hz, 1H), 7.92 (d, J = 2.6 Hz, 1H), 7.86 (s, 1H), 7.25-7.21 (m, 1H), 7.19- 7.12 (m, 1H), 6.95-6.88 (m, 2H), 6.20 (d, J = 8.5 Hz, 1H), 5.15-5.05 (m, 1H), 3.87 (s, 3H), 3.04-1.73 (m, 4H), 1.31 (s, 9H). |
| 42n | 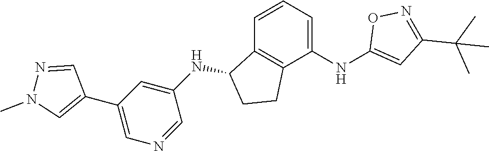 [SMILES: Cn1cc(-c2cncc(N[C@H]3CCc4c(Nc5cc(C(C)(C)C)no5)cccc43)c2)cn1] | 429.2 | ¹H NMR 62 MHz, CDCl₃) δ 8.01 (s, 2H), 7.82-7.55 (m, 2H), 7.38-6.92 (m, 4H), 6.44 (s, 1H), 5.45 (s, 1H), 5.31-4.86 (m, 1H), 3.96 (s, 3H), 2.84 (s, 4H), 1.33 (s, 9H). |
| 42o | 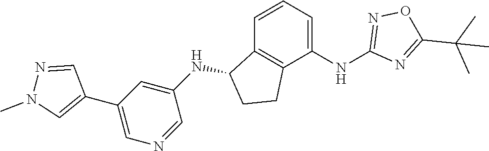 [SMILES: Cn1cc(-c2cncc(N[C@H]3CCc4c(Nc5noc(C(C)(C)C)n5)cccc43)c2)cn1] | 430.2 | ¹H NMR (62 MHz, CDCl₃) δ 8.13 (s, 1H), 8.06-7.79 (m, 2H), 7.75 (s, 1H), 7.64 (s, 1H), 7.50-7.17 (m, 2H), 7.16-6.94 (m, 2H), 6.45 (s, 1H), 5.25-4.87 (m, 1H), 3.97 (s, 3H), 3.17-1.70 (m, 4H), 1.46 (s, 9H). |
| 42p | 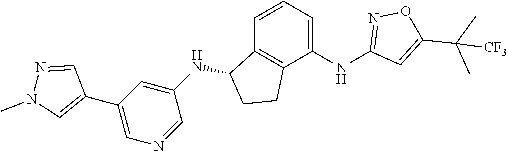 [SMILES: Cn1cc(-c2cncc(N[C@H]3CCc4c(Nc5cc(C(C)(C)C(F)(F)F)on5)cccc43)c2)cn1] | 483.2 | ^{I}H NMR (62 MHz, acetone-d₆) δ 8.36-7.91 (m, 3H), 7.90-7.65 (m, 2H), 7.48-6.82 (m, 4H), 6.29 (s, 1H), 5.56-4.88 (m, 2H), 3.91 (s, 3H), 3.30-1.74 (m, 4H), 1.58 (s, 6H). |
| 42q | 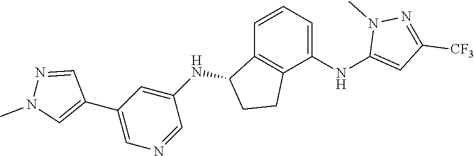 [SMILES: Cn1cc(-c2cncc(N[C@H]3CCc4c(Nc5cc(C(F)(F)F)nn5C)cccc43)c2)cn1] | 454.2 | 1H NMR (62 MHz, acetone-d₆) δ 8.14-7.75 (m, 4H), 7.39-6.81 (m, 4H), 6.74-6.52 (m, 1H), 6.28 (s, 1H), 5.45-4.92 (m, 2H), 3.92 (s, 3H), 3.78 (s, 3H), 3.14-1.61 (m, 4H). |
| 42r | 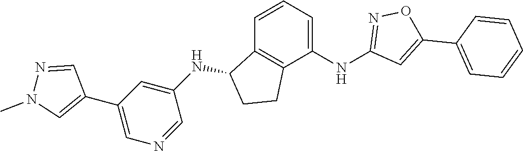 [SMILES: Cn1cc(-c2cncc(N[C@H]3CCc4c(Nc5cc(-c6ccccc6)on5)cccc43)c2)cn1] | 449.2 | 1H NMR (400 MHz, DMSO-d₆) δ 8.53 (s, 1H), 8.18-8.15 (m, 1H), 8.03 (d, J = 1.8 Hz, 1H), 7.93 (d, J = 2.6 Hz, 1H), 7.88-7.77 (m, 4H), 7.60-7.47 (m, 3H), 7.27-7.16 (m, 2H), 6.91 (d, J = 7.4 Hz, 1H), 6.72 (s, 1H), 6.21 (d, J = 8.5 Hz, 1H), 5.12 (q, J = 7.5 Hz, 1H), 3.87 (s, 3H), 3.05 (ddd, J = 15.9, 8.7, 3.6 Hz, 1H), 2.84 (dt, J = 15.8, 7.9 Hz, 1H), 2.61 (dtd, J = 11.4, 7.7, 3.6 Hz, 1H), 1.93- 1.76 (m, 1H). |
| 42s | 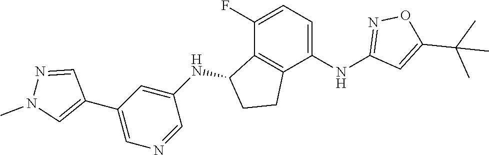 [SMILES: Cn1cc(-c2cncc(N[C@H]3CCc4c(Nc5cc(C(C)(C)C)on5)ccc(F)c43)c2)cn1] | 447.2 | 1H NMR (62 MHz, acetone-d₆) δ 8.45-7.48 (m, 6H), 7.38-7.18 (m, 1H), 7.15-6.69 (m, 1H), 5.90 (s, 1H), 5.50-5.13 (m, 2H), 3.91 (s, 3H), 3.35-1.83 (m, 4H), 1.32 (s, 9H). |
| 42t | 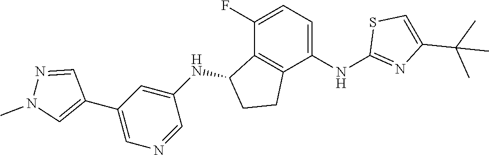 [SMILES: Cn1cc(-c2cncc(N[C@H]3CCc4c(Nc5nc(C(C)(C)C)cs5)ccc(F)c43)c2)cn1] | 463.2 | 1H NMR (62 MHz, CDCl₃) δ 8.20-8.05 (m, 1H), 8.04-7.87 (m, 1H), 7.86-7.52 (m, 3H), 7.15-6.74 (m, 2H), 6.21 (s, 1H), 5.45-5.02 (m, 1H), 4.32-4.04 (m, 1H), 3.95 (s, 3H), 3.23-1.89 (m, 4H), 1.31 (s, 9H). |

### Example 43

### N-(5-fluoro-1-((5-(1-methyl-1H-pyrazol-4-yl) pyridin-3-yl)amino)-2,3-dihydro-1H-inden-4-yl)-3-methylbenzamide

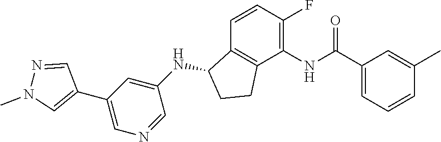

Compound 43 was prepared by the following steps:

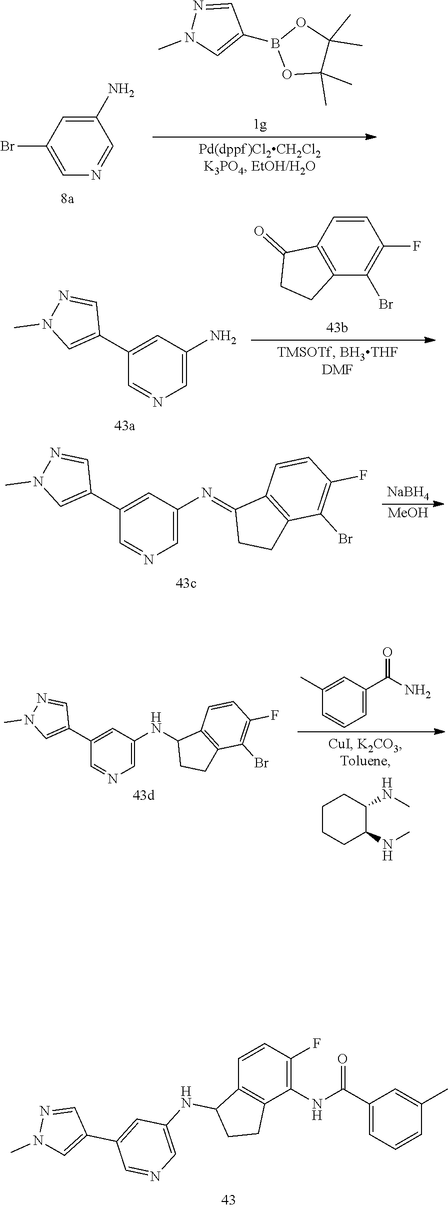

Step 1: The solution of 8a (10.0 g, 57.8 mmol), 1g (14.4 g, 69.4 mmol), Pd(dppf)Cl₂·CH₂Cl₂ (62 mg, 0.1 mmol) and K₃PO₄ (18.4 g, 86.7 mmol) in mixed solvent of EtOH (80 mL) and water (20 mL) was degassed with N₂, then heated to 85° C. (outer temperature) and stirred for 3 h. After concentration, the residue was diluted with water (200 mL), and extracted with EtOAc (200 mL*3). The combined organic layers were washed with brine (200 mL), dried over Na₂SO₄, filtered and concentrated. The residue was purified by silica gel chromatography to give product as a yellow solid (7.5 g, yield: 75%). ESI-MS (m/z): 175.1 [M+H]⁺.

Step 2: To a solution of 43a (175 mg, 1.0 mmol) and 43b (252 mg, 1.1 mmol) in DMF (1 mL) was added trimethylsilyl trifluoromethanesulfonate (TMSOTf) (556 mg, 2.5 mmol) and a solution of borane in THF (1 M, 1 mL, 1.0 mmol) in turn. The resulting mixture was stirred under N₂ at room temperature for 12 h. TLC analysis showed the reaction was complete. The mixture was diluted with EtOAc (8 mL), washed with 5% aqueous NaHCO₃ (4 mL) and purified by chromatography to give product as a yellow solid (104 mg, yield: 27%). ESI-MS (m/z): 385.1 [M+H]⁺.

Step 3: To a solution of 43c (97 mg, 0.25 mmol) in MeOH (2 mL) was added NaBH₄ (19 mg, 0.50 mmol). The resulting mixture was stirred at room temperature for 2 h. TLC analysis showed the reaction was complete. The reaction mixture was purified by chromatography to give product as a white solid (71 g, yield: 73%). ESI-MS (m/z): 387.1 [M+H]⁺.

Step 4: To a solution of 43d (70 mg, 0.18 mmol) and 3-Methylbenzamide (37 mg, 0.27 mmol) in toluene (0.5 mL) was added CuI (4 mg, 0.02 mmol), (1S, 2S)-(+)-N,N′-Dimethylcyclohexanediamine (3 mg, 0.02 mmol) and K₂CO₃ (50 mg, 0.36 mmol) in turn. The resulting mixture was degassed with N₂, and refluxed for 3 h. TLC analysis showed the reaction was complete. The reaction mixture was diluted with EtOAc (6 mL), washed with water (3 mL) and purified by silica gel chromatography to give product as a white solid (65 mg, yield: 81%). ESI-MS (m/z): 442.2 [M+H]⁺. ¹H NMR (400 MHz, DMSO-d₆) δ 10.04 (s, 1H), 8.17 (s, 1H), 7.93-7.73 (m, 3H), 7.43 (d, J=4.7 Hz, 2H), 7.36-7.05 (m, 3H), 6.31 (s, 1H), 5.15 (d, J=7.9 Hz, 1H), 3.87 (s, 3H), 2.89 (s, 1H), 2.79 (dt, J=16.4, 8.1 Hz, 1H), 2.61 (dd, J=13.9, 6.1 Hz, 1H), 2.41 (s, 3H), 1.83 (s, 1H).

### Example 44

The preparation of compounds 44a-44e refer to the synthesis of compound 43 in example 43.

TABLE 13

| | | MS | |
|---|---|---|---|
| | | [M + | |
| Cpds | Structure | 1]⁺ | ¹H NMR |
| 44a | 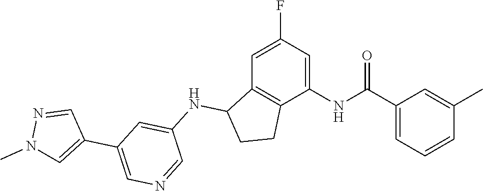 | 442.2 | ¹H NMR (400 MHz, DMSO-d₆) δ 10.01 (s, 1H), 8.18 (s, 1H), 8.14-8.01 (m, 1H), 7.94 (s, 1H), 7.88 (d, J = 0.9 Hz, 1H), 7.81-7.72 (m, 2H), 7.48-7.40 (m, 2H), 7.34 (dd, J = 10.9, 2.4 Hz, 1H), 7.26 (s, 1H), 6.90 (dd, J = 8.3, 2.3 Hz, 1H), 6.31 (d, J = 8.5 Hz, 1H), 5.16 (q, J = 7.8 Hz, 1H), 3.87 (s, 3H), 3.00-2.89 (m, 1H), 2.82 (dt, J = 16.1, 8.2 Hz, 1H), 2.61 (ddt, J = 12.8, 8.4, 4.1 Hz, 1H), 2.41 (s, 3H), 1.80 (dq, J = 12.2, 8.4 Hz, 1H). |
| 44b | 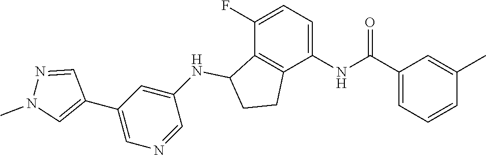 | 442.2 | ¹H NMR (400 MHz, DMSO-d₆) δ 9.98 (s, 1H), 8.16 (s, 1H), 8.03 (s, 1H), 7.92-7.83 (m, 2H), 7.79 (s, 2H), 7.42 (s, 3H), 7.19 (s, 1H), 7.07-7.02 (m, 1H), 6.33 (d, J = 7.9 Hz, 1H), 5.30 (s, 1H), 3.88 (s, 3H), 3.05- 3.03 (m, 1H), 2.88 (s, 1H), 2.41 (s, 3H), 1.96 (s, 1H), 0.85 (s, 1H). |
| 44c | 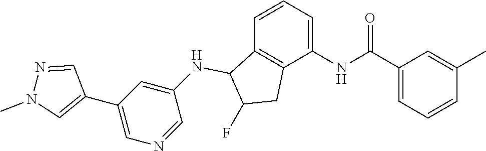 | 442.2 | ¹H NMR (400 MHz, CDCl₃) δ 8.24 (s, 1H), 8.15 (s, 1H), 7.90-7.55 (m, 6H), 7.43 (d, J = 4.6 Hz, 2H), 7.35 (t, J = 7.6 Hz, 1H), 7.23 (d, J = 29.6 Hz, 2H), 5.53 (d, J = 53.6 Hz, 1H), 5.30-5.11 (m, 1H), 4.47 (d, J = 9.9 Hz, 1H), 3.99 (s, 3H), 3.21 (dd, J = 23.4, 17.2 Hz, 2H), 2.48 (s, 3H). |
| 44d | 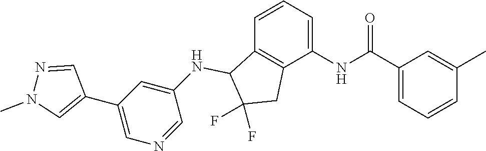 | 486.2 | ¹H NMR (400 MHz, DMSO-d₆) δ 10.16 (s, 1H), 8.15 (s, 1H), 8.10 (dd, J = 9.6, 2.2 Hz, 2H), 7.86 (d, J = 0.8 Hz, 1H), 7.74 (dt, J = 7.7, 1.4 Hz, 1H), 7.67 (d, J = 1.8 Hz, 1H), 7.48-7.30 (m, 5H), 7.19 (d, J = 7.5 Hz, 1H), 6.54 (d, J = 9.6 Hz, 1H), 5.68 (q, J = 10.1 Hz, 1H), 3.88 (s, 3H), 3.52-3.45 (m, 2H), 2.04 (ddd, J = 13.5, 8.5, 5.1 Hz, 1H), 1.05-0.99 (m, 2H), 0.81-0.76 (m, 2H). |
| 44e | 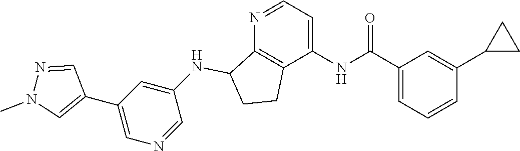 [SMILES: Cn1cc(-c2cncc(NC3CCc4c(NC(=O)c5cccc(C6CC6)c5)ccnc43)c2)cn1] | 451.2 | ¹H NMR (400 MHz, DMSO-d₆) δ 10.16 (s, 1H), 8.35-8.34 (d, J = 5.2 Hz, 1H), 8.16 (s, 1H), 8.05-8.04 (d, J = 2.0 Hz, 1H), 7.98-7.97 (d, J = 2.0 Hz, 1H), 7.87 (s, 1H), 7.73-7.71 (dd, J = 8.0, 2.0 Hz, 1H), 7.65 (dd, J = 2.0, 2.0 Hz, 1H), 7.58-7.57 (d, J = 5.2 Hz, 1H), 7.45-7.41 (dd, J = 8.0, 8.0 Hz, 1H), 7.35-7.32 (m, 2H), 6.26- 6.24 (d, J = 6.4 Hz, 1H), 5.05-4.99 (dd, J = 14.4, 6.4 Hz, 1H), 3.88 (s, 3H), 3.04- |
| | | | 2.98 (m, 1H), 2.94-2.86 (m, 1H), 2.68- |
| | | | 2.63 (m, 1H), 2.06-2.01 (m, 1H), 1.91- |
| | | | 1.80 (m, 1H), 1.05-1.00 (m, 2H), 0.80- |
| | | | 0.76 (m, 2H). |

### Example 45

### (S)—N-(7-fluoro-1-((5-(1-methyl-1H-pyrazol-4-yl) pyridin-3-yl)amino)-2,3-dihydro-1H-inden-4-yl)-3-methylbenzamide

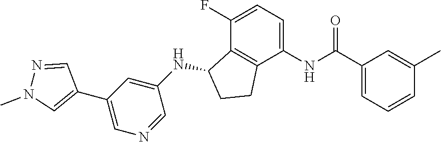

Compound 45 was prepared by the following steps:

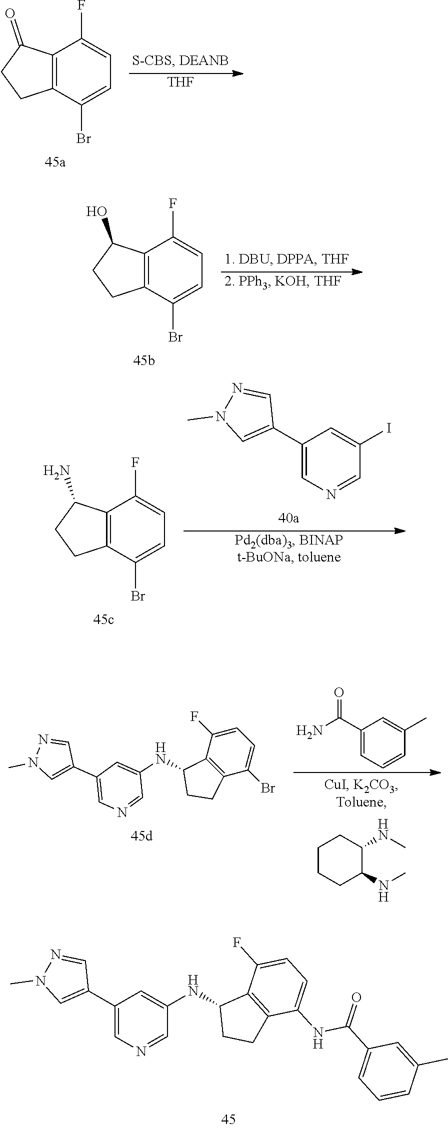

Step 1: To a stirred solution of DEANB (3.26 g, 20.0 mmol) and(S)-(−)-2-Methyl-CBS-oxazaborolidine (1 M in toluene, 2 mL, 2.0 mmol) in THF (10 mL) cooled to 0° C. under N₂ was added a solution of 7-Bromo-4-fluoro-2,3-dihydro-1H-inden-1-one 45a (4.58 g, 20.0 mmol) in THF (50 mL) dropwise during a period of 3 h. The reaction mixture was stirred at 0° C. until TLC analysis showed the reaction was complete. The mixture was quenched by slow addition of MeOH (5 mL) at 0° C., then allowed to warm to room temperature and stirred for another 1 h. The mixture was concentrated to dryness and redissolved in EtOAc (100 mL), then washed with 10% aqueous H₂SO₄ (50 mL*2), saturated aqueous NaHCO₃ (50 mL) and brine (50 mL) in turn, then dried over Na₂SO₄ and concentrated to dryness under reduced pressure to give product as a light brown solid. The crude product was used directly for the next step without further purification (Yield: 100%).

Step 2: To a solution of 45b (3.30 g, 20.0 mmol) in THF (35 mL) was added DBU (7.6 g, 50.0 mmol), followed by addition of DPPA (11.0 g, 40.0 mmol) at 0° C. The resulting mixture was heated to 30° C. and stirred for 12 h. TLC analysis showed the reaction was complete. The mixture was concentrated to dryness and purified by chromatography to give a azide intermediate. To the above azide in THF (100 mL) was added water (50 mL) at 0° C., followed by addition of PPh₃ (15.72 g, 60.0 mmol) and KOH (3.36 g, 60.0 mmol). The reaction mixture was allowed to warm to room temperature and stirred for 12 h. TLC analysis showed the reaction was complete. The reaction mixture was poured into separatory funnel and separated. The aqueous layer was discarded, and the organic layer was concentrated to dryness. Water (100 mL) was added to the above residue, the pH was adjusted between 1-2 with 6N HCl (aq.). The acidic aqueous solution was wash with CH₂Cl₂ (100 mL*2), then adjusted to pH>13 with 10% aqueous NaOH. The resulting suspension was extracted with CH₂Cl₂ (100 mL), the aqueous layer was discarded and the organic layer was dried over Na₂SO₄, and concentrated to dryness to give crude product (3.20 g, yield: 70%).

Step 3: To a solution of 40a (850 mg, 3.0 mmol), 45c (690 mg, 3.0 mmol), Pd₂(dba)₃ (137 mg, 0.15 mmol) and BINAP (373 mg, 0.6 mmol) in toluene (3 mL) was added t-BuONa (864 mg, 9.0 mmol). The resulting mixture was degassed with N₂ for three times, then heated to 90° C. and stirred until TLC analysis showed the reaction was complete. After natural cooling to room temperature, EtOAc (30 mL) was added to the mixture. The mixture was washed with water (30 mL), dried over Na₂SO₄, and concentrated to dryness. The residue was purified by chromatography to give product (221 mg, yield: 19%). ESI-MS (m/z): 387.2 [M+H]⁺.

Step 4: The mixture of 45d (77 mg, 0.2 mmol) 3-Methylbenzamide (82 mg, 0.6 mmol), K₂CO₃ (83 mg, 0.6 mmol), CuI (4 mg, 0.02 mmol), and (1S, 2S)-(+)-N,N′-Dimethylcyclohexanediamine (6 mg, 0.04 mmol) in toluene (0.2 mL) was degassed with N₂ for three times, then heated at 90° C. for 3 h. TLC analysis showed the reaction was complete. After natural cooling to room temperature, the reaction mixture was diluted with EtOAc (10 mL), and washed with 12% aqueous ammonia (10 mL) and water (10 mL), dried over Na₂SO₄ and concentrated to dryness under reduced pressure. The residue was purified by chromatography to give product (61 mg, yield: 68%). ESI-MS (m/z): 442.5 [M+H]⁺. ¹H NMR (400 MHz, DMSO-d₆) δ 10.00 (s, 1H), 8.16 (s, 1H), 8.04 (s, 1H), 7.90 (s, 1H), 7.88 (s, 1H), 7.81-7.76 (m, 2H), 7.45-7.40 (m, 3H), 7.21-7.20 (m, 1H), 7.05 (t, J=8.8 Hz, 1H), 6.35 (d, J=8.1 Hz, 1H), 5.33-5.28 (m, 1H), 3.88 (s, 3H), 3.10-3.02 (m, 1H), 2.91-2.83 (m, 1H), 2.49-2.44 (m, 1H), 2.41 (s, 3H), 2.01-1.93 (m, 1H).

### Example 46

The preparation of compounds 46a-46d refer to the synthesis of compound 45 in example 45.

TABLE 14

| | | MS | |
|---|---|---|---|
| Cpds | Structure | [M + 1]⁺ | ¹H NMR |
| 46a | 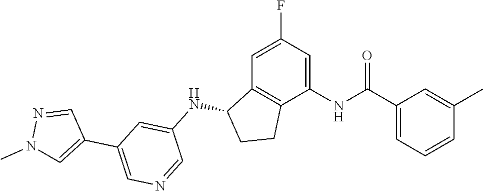 | 442.2 | ¹H NMR (400 MHz, DMSO-d₆) δ 10.01 (s, 1H), 8.18 (d, J = 0.8 Hz, 1H), 8.06 (d, J = 1.8 Hz, 1H), 7.94 (d, J = 2.6 Hz, 1H), 7.88 (d, J = 0.8 Hz, 1H), 7.83-7.72 (m, 2H), 7.50-7.41 (m, 2H), 7.34 (dd, J = 10.9, 2.4 Hz, 1H), 7.26 (t, J = 2.2 Hz, 1H), 6.96-6.86 (m, 1H), 6.30 (d, J = 8.5 Hz, 1H), 5.16 (q, J = 7.8 Hz, 1H), 3.87 (s, 3H), 2.93 (ddd, J = 11.8, 9.1, 4.6 Hz, 1H), 2.83 (dt, J = 16.1, 8.3 Hz, 1H), 2.61 (ddt, J = 12.4, 8.0, 3.9 Hz, 1H), 2.41 (s, 3H), 1.81 (dq, J = 12.2, 8.6 Hz, 1H). |
| 46b | 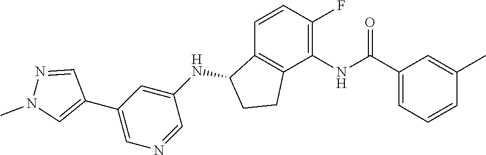 | 442.2 | ¹H NMR (400 MHz, DMSO-d₆) δ 10.05 (s, 1H), 8.17 (s, 1H), 8.04 (d, J = 1.8 Hz, 1H), 7.93 (d, J = 2.6 Hz, 1H), 7.88 (d, J = 0.8 Hz, 1H), 7.85-7.75 (m, 2H), 7.48-7.39 (m, 2H), 7.25 (t, J = 2.3 Hz, 1H), 7.20 (dd, J = 8.3, 4.5 Hz, 1H), 7.13 (dd, J = 10.1, 8.2 Hz, 1H), 6.30 (d, J = 8.5 Hz, 1H), 5.15 (q, J = 7.7 Hz, 1H), 3.87 (s, 4H), 2.90 (ddd, J = 16.3, 8.7, 3.5 Hz, 1H), 2.78 (dt, J = 16.4, 8.2 |
| | | | Hz, 1H), 2.59 (dp, J = 12.1, 3.7 Hz, |
| | | | 1H), 2.41 (s, 3H), 1.83 (dq, J = 12.4, |
| | | | 8.5 Hz, 1H). |
| 46c | 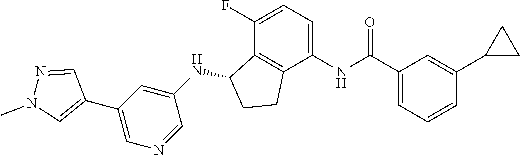 [SMILES: Cn1cc(-c2cncc(N[C@H]3CCc4c(NC(=O)c5cccc(C6CC6)c5)ccc(F)c43)c2)cn1] | 468.2 | ¹H NMR (400 MHz, DMSO-d₆) δ 9.98 (s, 1H), 8.16 (s, 1H), 8.03 (d, J = 1.8 Hz, 1H), 7.91-7.84 (m, 2H), 7.73 (dt, J = 7.8, 1.5 Hz, 1H), 7.65 (t, J = 1.8 Hz, 1H), 7.45-7.35 (m, 2H), 7.31 (dt, J = 7.8, 1.5 Hz, 1H), 7.20 (s, 1H), 7.05 (t, J = 8.8 Hz, 1H), 6.34 (d, J = 8.1 Hz, 1H), 5.30 (td, J = 7.7, 4.3 Hz, 1H), 3.88 (s, 3H), 3.05 (dt, J = 15.6, 7.6 Hz, 1H), 2.93-2.79 (m, 1H), 2.49-2.39 t(m, 1H), 2.03 (td, J = |
| | | | 8.4, 4.2 Hz, 1H), 1.96 (ddt, J = 13.0, 8.7, |
| | | | 3.8 Hz, 1H), 1.05-0.98 (m, 2H), 0.80- |
| | | | 0.74 (m, 2H). |
| 46d | 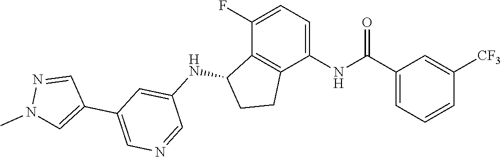 [SMILES: Cn1cc(-c2cncc(N[C@H]3CCc4c(NC(=O)c5cccc(C(F)(F)F)c5)ccc(F)c43)c2)cn1] | 496.2 | ¹H NMR (400 MHz, DMSO-d₆) δ 10.30 (s, 1H), 8.35-8.26 (m, 2H), 8.16 (s, 1H), 8.07-7.96 (m, 2H), 7.92-7.85 (m, 2H), 7.80 (t, J = 7.8 Hz, 1H), 7.40 (dd, J = 8.6, 4.4 Hz, 1H), 7.20 (t, J = 2.2 Hz, 1H), 7.07 (t, J = 8.7 Hz, 1H), 6.34 (d, J = 8.1 Hz, 1H), 5.35-5.27 (m, 1H), 3.87 (s, 4H), 3.06 (dt, J = 15.7, 7.6 Hz, 1H), 2.93-2.81 (m, 1H), 1.96 (ddt, J = 13.1, 8.9, 4.8 Hz, 1H). |

### Example 47

### N-(3-((5-(1-methyl-1H-pyrazol-4-yl) pyridin-3-yl)amino)-2,3-dihydrobenzofuran-7-yl)-4-(trifluoromethyl)benzamide

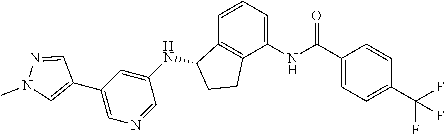 [SMILES: Cn1cc(-c2cncc(N[C@H]3CCc4c(NC(=O)c5ccc(C(F)(F)F)cc5)cccc43)c2)cn1]

Compound 47 was prepared by the following steps:

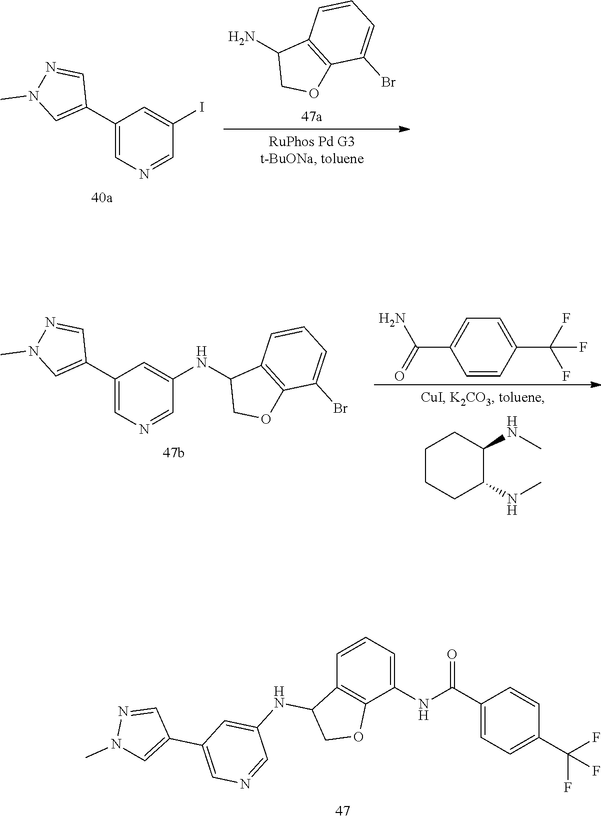

Step 1: 40a (256 mg, 0.9 mmol), 47a (150 mg, 0.6 mmol), RuPhosPdG3 (25.2 mg, 0.03 mmol) and t-BuONa (231 mg, 2.4 mmol) were added to toluene (3 mL). The resulting mixture was heated to 90° C. under N₂ atmosphere for 2 h. When TLC analysis showed the reaction was complete, the reaction was concentrated to dryness and purified by chromatography to give product (46 mg, yield: 16.0%). ESI-MS (m/z): 371.1 [M+H]⁺.

Step 2: 47b (46 mg, 0.12 mmol), 4-trifluoromethylbenzenamide (70 mg, 0.37 mmol), CuI (25.2 mg, 0.03 mmol), (1R, 2R)-(−)-N,N′-dimethyl-1,2-cyclohexandiamideand (4 mg, 0.025 mmol) and K₂CO₃ (51 mg, 0.37 mmol) were added to toluene (0.12 mL). The resulting mixture was heated to 90° C. under N₂ atmosphere for 1 h. When TLC analysis showed the reaction was complete, EtOAc (30 mL) was added to the reaction. The mixture was washed twice with ammonia (10 ml) and water (10 ml) in turn, dried over Na₂SO₄ and concentrated to dryness, purified by chromatography to give product (20 mg, yield: 34%). ESI-MS (m/z): 480.6 [M+H]⁺. ¹H NMR (400 MHz, DMSO-d₆) δ 10.23 (s, 1H), 8.16 (d, J=7.4 Hz, 3H), 8.08 (d, J=1.8 Hz, 1H), 7.91 (dd, J=11.4, 2.7 Hz, 4H), 7.49 (d, J=8.0 Hz, 1H), 7.29-7.19 (m, 2H), 6.95 (t, J=7.7 Hz, 1H), 6.47 (d, J=7.9 Hz, 1H), 5.47 (q, J=3.0 Hz, 1H), 4.87 (dd, J=9.5, 7.8 Hz, 1H), 4.31 (dd, J=9.5, 4.7 Hz, 1H), 3.87 (s, 3H).

### Example 48

### N⁴-(5-(tert-butyl) isoxazol-3-yl)-N¹-(5-(1-methyl-1H-pyrazol-4-yl) pyridin-3-yl)-2,3-dihydro-1H-indene-1,4-diamine

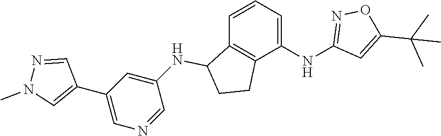 [SMILES: Cn1cc(-c2cncc(NC3CCc4c(Nc5cc(C(C)(C)C)on5)cccc43)c2)cn1]

Compound 48 was prepared by the following steps:

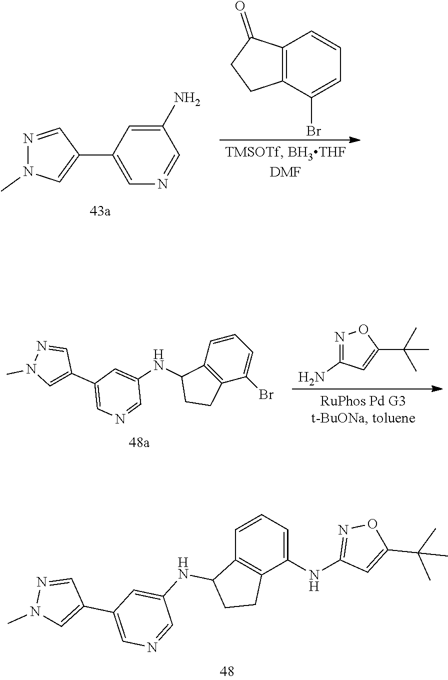

Step 1: To a stirred solution of compound 43a (2.6 g, 15.0 mmol), 4-bromoindanone (3.5 g, 16.5 mmol) in DMF (30 mL) was added trimethylsilyl trifluoromethanesulfonate (TMSOTf) (8.3 g, 37.5 mmol) and a 1 M borane tetrahydrofuran solution (30 mL, 30 mmol) at 0° C. The reaction was stirred at 0° C. for 10 min before being warmed to room temperature and stirred for 18 h. Then 100 mL of water was added to the reaction solution and the pH was adjusted to 9˜10 with aqueous KOH (5%). The resulting mixture was extracted with ethyl acetate (100 mL*3). The combined organic phases were washed sequentially with water (150 mL*2) and brine (150 mL), dried over anhydrous sodium sulfate, filtered, and concentrated. The residue was purified by silica gel column chromatography to afford compound 48a (2.0 g, yield: 36%) as a black viscous substance. ESI-MS (m/z): 369.1 [M+H]⁺.

Step 2: Similar procedure to step 3 of example 41 was followed, with 65% yield.

ESI-MS (m/z): 429.2 [M+H]⁺. ¹H NMR (62 MHz, acetone-d₆) δ 8.24-7.71 (m, 5H), 7.68-7.04 (m, 3H), 7.04-6.82 (m, 1H), 5.93 (s, 1H), 5.51-4.91 (m, 2H), 3.91 (s, 3H), 3.18-1.60 (m, 4H), 1.32 (s, 9H).

### Example 49

### (S)—N⁴-(5-(tert-butyl) isoxazol-3-yl)-N¹-methyl-N1-(5-(1-methyl-1H-pyrazol-4-yl) pyridin-3-yl)-2,3-dihydro-1H-indene-1,4-diamine

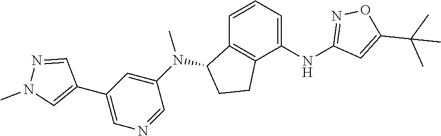 [SMILES: CN(c1cncc(-c2cnn(C)c2)c1)[C@H]1CCc2c(Nc3cc(C(C)(C)C)on3)cccc21]

Preparation of compound 49 referring to the synthesis of compound 20 of example 20.

ESI-MS (M/S): 443.2 [M+H]⁺. ¹H NMR (62 MHz, CDCl₃) δ 8.41-8.01 (m, 2H), 7.89-7.32 (m, 4H), 7.23-6.72 (m, 2H), 6.10 (s, 1H), 5.82 (s, 1H), 5.58 (t, J=7.7 Hz, 1H), 3.96 (s, 3H), 3.12-1.77 (m, 7H), 1.35 (s, 9H).

### Example 50

### (S)—N-(3-cyclopropylphenyl)-7-fluoro-1-((5-(1-methyl-1H-pyrazol-4-yl) pyridin-3-yl)amino)-2,3-dihydro-1H-indene-4-carboxamide

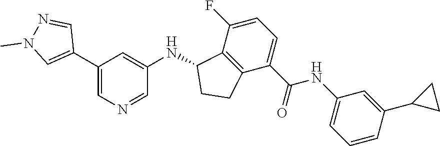 [SMILES: Cn1cc(-c2cncc(N[C@H]3CCc4c(C(=O)Nc5cccc(C6CC6)c5)ccc(F)c43)c2)cn1]

Compound 50 was prepared by the following steps:

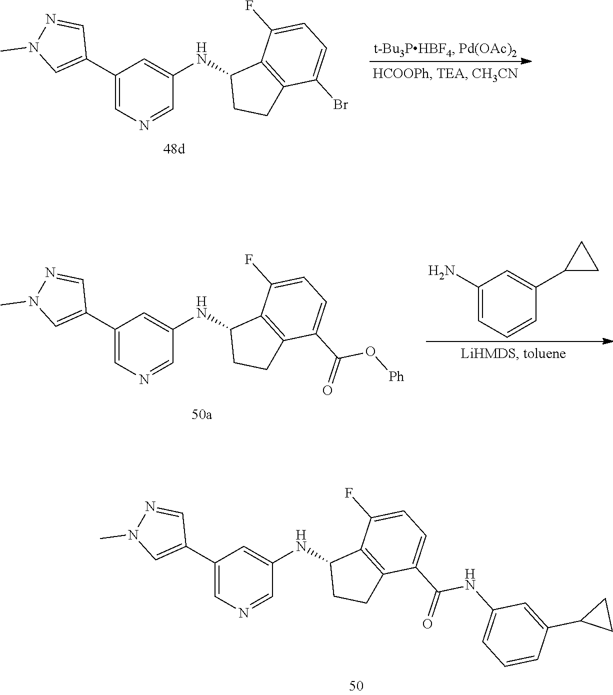 [SMILES: Cn1cc(-c2cncc(N[C@H]3CCc4c(Br)ccc(F)c43)c2)cn1 ; Cn1cc(-c2cncc(N[C@H]3CCc4c(C(=O)Oc5ccccc5)ccc(F)c43)c2)cn1 ; Nc1cccc(C2CC2)c1 ; Cn1cc(-c2cncc(N[C@H]3CCc4c(C(=O)Nc5cccc(C6CC6)c5)ccc(F)c43)c2)cn1]

Step 1: To a stirred solution of compound 45d (1000 mg, 2.60 mmol), and tri-tert-butylphosphonium tetrafluoroborate (188 mg, 0.65 mmol) in acetonitrile (5 mL) was added triethylamine (525 mg, 5.2 mmol), phenyl formate (634 mg, 5.2 mmol), and palladium acetate (29 mg, 0.13 mmol). The resulting mixture was degassed with N₂ for three times and stirred at 80° C. until TLC showed the reaction was complete, then concentrated to dryness. The residue was purified on an ISCO chromatography to afford compound 50a (755 mg, yield: 68%).

Step 2: To a stirred solution of compound 50a (43 mg, 0.1 mmol), 3-cyclopropylaniline (27 mg, 0.2 mmol) in toluene (1.0 mL) was added LiHMDS (0.3 mL, 1N in THF, 0.3 mmol). The resulting mixture was stirred at 25° C. until the reaction was complete detected by TLC, and then quenched with saturated aq. NH₄Cl (10 mL). The resulting mixture was extracted with EtOAc (5 mL). The organic phases were dried over anhydrous sodium sulfate and concentrated to dryness, The residue was purified on an ISCO chromatography to afford compound 50 (28 mg, yield: 59%). ESI-MS (m/z): 468.2 [M+H]⁺. ¹H NMR (400 MHz, DMSO-d₆) δ 10.14 (s, 1H), 8.16 (s, 1H), 8.04 (d, J=1.8 Hz, 1H), 7.88 (d, J=2.8 Hz, 2H), 7.73 (dd, J=8.5, 4.8 Hz, 1H), 7.57-7.51 (m, 1H), 7.47 (t, J=1.9 Hz, 1H), 7.27-7.13 (m, 3H), 6.88-6.81 (m, 1H), 6.29 (d, J=8.3 Hz, 1H), 5.30 (td, J=7.7, 3.8 Hz, 1H), 3.88 (s, 3H), 3.29 (t, J=7.9 Hz, 1H), 3.12 (ddd, J=17.1, 8.7, 4.7 Hz, 1H), 1.99 (tt, J=8.3, 4.2 Hz, 1H), 1.91 (ddd, J=13.4, 8.7, 5.2 Hz, 1H), 0.99-0.93 (m, 2H), 0.67-0.62 (m, 2H).

### Example 51

The preparation of compounds 51a-51b refers to the synthesis of compound 50 in example 50.

TABLE 15

| | | MS | |
|---|---|---|---|
| Cpds | Structure | [M + 1]⁺ | ¹H NMR |
| 51a | 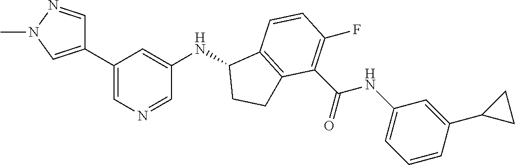 [SMILES: Cn1cc(-c2cncc(N[C@H]3CCc4c3ccc(F)c4C(=O)Nc3cccc(C4CC4)c3)c2)cn1] | 468.2 | ¹H NMR (400 MHz, DMSO-d₆) δ 10.44 (s, 1H), 8.17 (s, 1H), 8.05 (d, J = 1.8 Hz, 1H), 7.93 (d, J = 2.6 Hz, 1H), 7.88 (s, 1H), 7.53-7.45 (m, 2H), 7.39 (dd, J = 8.4, 4.9 Hz, 1H), 7.26 (t, J = 2.2 Hz, 1H), 7.22 (t, J = 7.8 Hz, 1H), 7.16 (dd, J = 9.9, 8.4 Hz, 1H), 6.87-6.82 (m, 1H), 6.25 (d, J = 8.3 Hz, 1H), 5.11 (q, J = 7.5 Hz, 1H), 3.87 (s, 4H), 2.91 (dt, J = 16.3, 8.0 Hz, 1H), 1.94- 1.83 (m, 3H), 1.00-0.93 (m, 2H), 0.68-0.62 (m, 2H). |
| 51b | 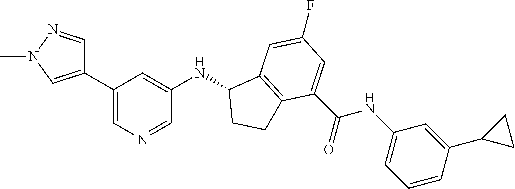 [SMILES: Cn1cc(-c2cncc(N[C@H]3CCc4c(C(=O)Nc5cccc(C6CC6)c5)cc(F)cc43)c2)cn1] | 468.2 | ¹H NMR (400 MHz, DMSO-d₆) δ 10.19 (s, 1H), 8.18 (s, 1H), 8.07 (d, J = 1.8 Hz, 1H), 7.94 (d, J = 2.6 Hz, 1H), 7.88 (s, 1H), 7.54 (d, J = 8.2 Hz, 1H), 7.50-7.43 (m, 2H), 7.30-7.18 (m, 3H), 6.85 (d, J = 7.8 Hz, 1H), 6.30 (d, J = 8.5 Hz, 1H), 5.14 (q, J = 7.7 Hz, 1H), 3.87 (s, 3H), 3.19 (dq, J = 13.6, 5.6, 4.6 Hz, 1H), 3.00 (dt, J = 16.5, 8.2 Hz, 1H), 2.74-2.57 (m, 1H), 1.91 (ddd, J = 13.3, 8.5, 5.0 Hz, 1H), 1.82 (dt, J = 12.3, 8.1 Hz, 1H), 1.02-0.93 (m, 2H), 0.70-0.62 (m, 2H). |

### Example 52

### (S)—N-(3-cyclopropylphenyl)-6-fluoro-1-(methyl (5-(1-methyl-1H-pyrazol-4-yl) pyridin-3-yl)amino)-2,3-dihydro-1H-indene-4-carboxamide

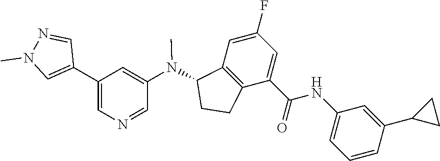 [SMILES: CN(c1cncc(-c2cnn(C)c2)c1)[C@H]1CCc2c(C(=O)Nc3cccc(C4CC4)c3)cc(F)cc21]

The preparation of compound 52 refers to the synthesis of compound 20 in example 20.

ESI-MS (m/z): 482.55 [M+H]⁺.

¹H NMR (400 MHz, DMSO-d₆) δ 10.21 (s, 1H), 8.25 (s, 1H), 8.19 (d, J=1.6 Hz, 1H), 8.15 (d, J=2.8 Hz, 1H), 7.97 (s, 1H), 7.54 (d, J=8.0 Hz, 1H), 7.51-7.45 (m, 3H), 7.22 (t, J=7.8 Hz, 1H), 7.11 (dd, J=8.3, 2.4 Hz, 1H), 6.85 (d, J=7.8 Hz, 1H), 5.74 (d, J=8.0 Hz, 1H), 3.88 (s, 3H), 3.18 (d, J=8.6 Hz, 1H), 3.01 (dt, J=16.9, 8.5 Hz, 1H), 2.68 (s, 3H), 2.47-2.39 (m, 1H), 2.05-1.96 (m, 1H), 1.92 (tt, J=8.7, 4.9 Hz, 1H), 1.00-0.94 (m, 2H), 0.68-0.63 (m, 2H).

### Example 53

### (S)—N-(3-cyclopropylphenyl)-5-fluoro-1-((5-(1-methyl-1H-pyrazol-4-yl) pyridin-3-yl)oxy)-2,3-dihydro-1H-indene-4-carboxamide

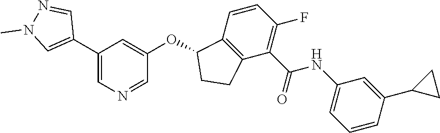 [SMILES: Cn1cc(-c2cncc(O[C@H]3CCc4c3ccc(F)c4C(=O)Nc3cccc(C4CC4)c3)c2)cn1]

Compound 53 was prepared by the following steps:

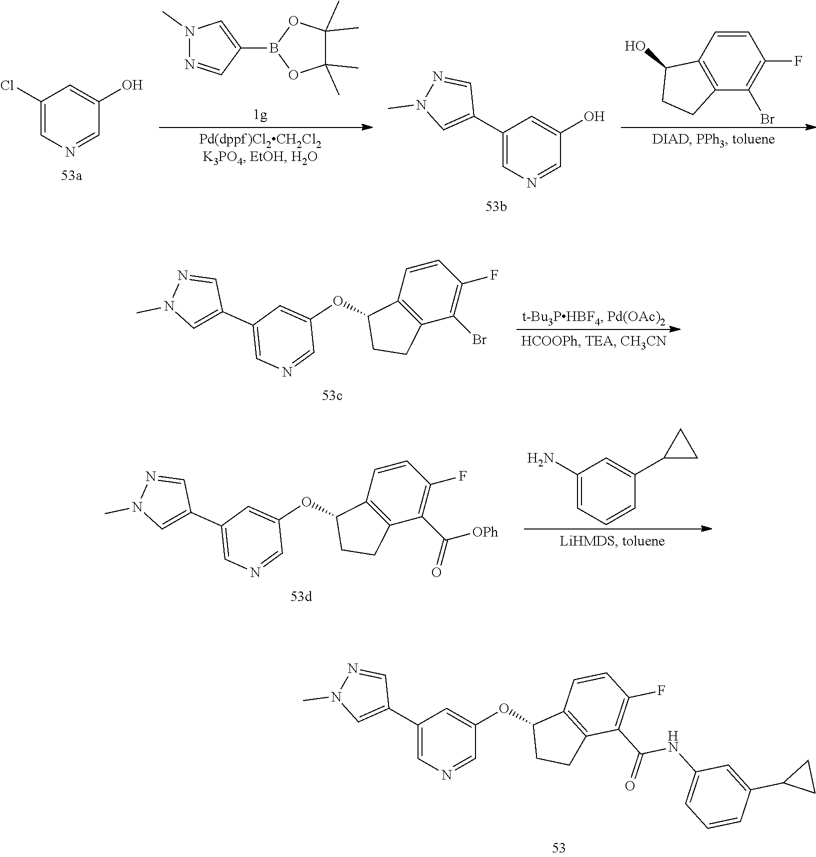

Step 1: To the reaction flask with ethanol (50 mL) and water (25 mL) was added compound 53a (6.5 g, 50.2 mmol), compound 1g (12.5 g, 60.2 mmol), potassium phosphate (16.0 g, 75.3 mmol), and Pd(dppf)Cl₂·CH₂Cl₂ (407 mg, 0.50 mmol) sequentially. The resulting mixture was degassed with N₂, then heated to 85° C. and stirred for 3 h. The reaction solution was concentrated to remove ethanol, then was diluted with water (130 mL), and extracted with ethyl acetate (130 mL*3). The combined organic phases were washed with brine (130 mL), dried over anhydrous sodium sulfate, filtered, and concentrated. The residue was purified by silica gel column chromatography to afford compound 53b (6.6 g, 75%) as a yellow solid. ESI-MS (m/z): 176.1 [M+H]⁺.

Step 2: To a solution of compound 53b (6.6 g, 37.7 mmol), (1R)-4-bromo-5-fluoro-2,3-dihydro-1H-inden-1-ol (10.4 g, 45.2 mmol), and PPh₃ (14.8 g, 56.5 mmol) in toluene (60 mL) cooled to −10° C. was slowly added dropwise DIAD (1.90 g, 9.4 mmol) under nitrogen, while keeping internal temperature at −10° C. After completion of the addition, the mixture was allowed to react for another 1 h. TLC showed the reaction was complete. Then water (1 mL) was added to the reaction mixture. The resulting mixture was allowed to warm to room temperature and stirred for 30 min. The suspension was filtered, and the filter cake was washed with toluene. The combined filtrates were concentrated to dryness. The residue was purified by column chromatography to obtain the compound 53c (7.7 g, yield: 53%). ESI-MS (m/z). 389.1 [M+H]⁺.

Step 3 and Step 4: Similar procedure to compound 50 in example 50 was followed to arrive at compound 53 (20.2 mg, 46%). ESI-MS (m/z): 469.2 [M+H]. ¹H NMR (400 MHz, DMSO-d₆) δ 10.44 (s, 1H), 8.17 (s, 1H), 8.05 (d, J=1.8 Hz, 1H), 7.93 (d, J=2.6 Hz, 1H), 7.88 (s, 1H), 7.53-7.45 (m, 2H), 7.39 (dd, J=8.4, 4.9 Hz, 1H), 7.26 (t, J=2.2 Hz, 1H), 7.22 (t, J=7.8 Hz, 1H), 7.16 (dd, J=9.9, 8.4 Hz, 1H), 6.87-6.82 (m, 1H), 6.25 (d, J=8.3 Hz, 1H), 5.11 (q, J=7.5 Hz, 1H), 3.87 (s, 4H), 2.91 (dt, J=16.3, 8.0 Hz, 1H), 1.94-1.83 (m, 3H), 1.00-0.93 (m, 2H), 0.68-0.62 (m, 2H).

### Example 54

### (S)—N-(3-cyclopropylphenyl)-5-fluoro-1-((5-(1-methyl-1H-pyrazol-4-yl) pyridin-3-yl)oxy)-2,3-dihydro-1H-indene-4-carboxamide

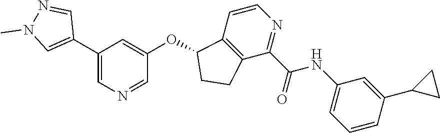 [SMILES: Cn1cc(-c2cncc(O[C@H]3CCc4c3ccnc4C(=O)Nc3cccc(C4CC4)c3)c2)cn1]

The preparation of compound 54 refers to the synthesis of compound 53 in example 53.

ESI-MS (m/z): 452.2 [M+H]⁺. ¹H NMR (400 MHZ. DMSO-d₆) δ 10.53 (s, 1H), 8.65 (d, J=4.8 Hz, 1H), 8.50 (d, J=1.6 Hz, 1H), 8.32 (s, 1H), 8.23 (d, J=2.7 Hz, 1H), 8.03 (s, 1H), 7.77-7.66 (m, 3H), 7.61 (d, J=2.0 Hz, 1H), 7.23 (t, J=7.9 Hz, 1H), 6.86 (d, J=8.1 Hz, 1H), 6.15 (t, J=6.0 Hz, 1H), 3.89 (s, 3H), 3.55 (ddd, J=17.8, 8.8, 4.9 Hz, 2H), 2.74 (dq, J=13.5, 7.9 Hz, 1H), 2.10 (dq, J=14.0, 6.2 Hz, 1H), 1.91 (td, J=8.6, 4.3 Hz, 1H), 1.00-0.94 (m, 2H), 0.71-0.66 (m, 2H).

### Example 55

### (S)—N-(3-cyclopropylphenyl)-7-((5-(1-methyl-1H-pyrazol-4-yl) pyridin-3-yl)amino)-6,7-dihydro-5H-cyclopenta[b]pyridine-4-carboxamide

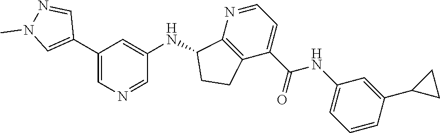 [SMILES: Cn1cc(-c2cncc(N[C@H]3CCc4c(C(=O)Nc5cccc(C6CC6)c5)ccnc43)c2)cn1]

Compound 55 was prepared by the following steps:

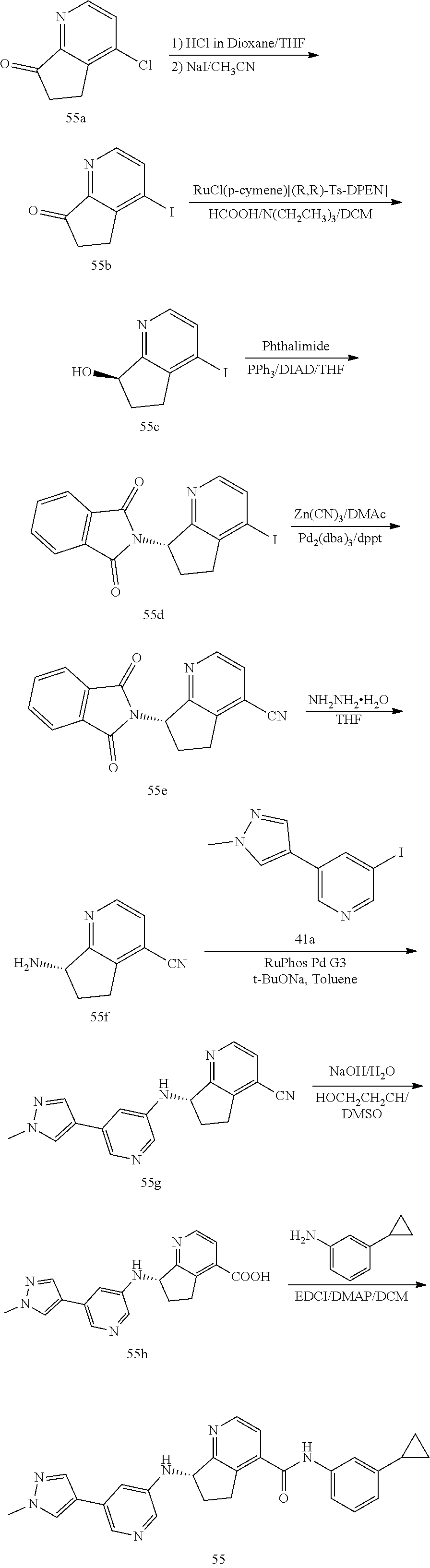

Step 1: To a stirred solution of compound 55a (586 mg, 3.5 mmol) in THF (5 mL) was added a solution of 4 M HCl in dioaxne (7 mL, 7 mmol). The mixture was stirred at room temperature for 0.5 h, and then evaporated to remove the solvent under reduced pressure. To the residue was added sodium iodide (3.49 g, 21 mmol) and CH₃CN (10 mL). The resulting mixture was stirred at 80° C. overnight. The reaction was complete detected by TLC. The mixture was partitioned between water and EtOAc. The separated organic phase was concentrated, then purified on an ISCO chromatography to obtain the 55b (726 mg, yield: 80.1%). ESI-MS (m/z): 260.0 [M+1]⁺.

Step 2: To a stirred solution of compound 55b (726 mg, 2.8 mmol), Et₃N (566 mg, 5.6 mmol), and RuCl(p-cymene) [(R,R)-Ts-DPEN] (89 mg, 0.14 mmol) in CH₂Cl₂ (20 mL) was added formic acid (386 mg, 8.4 mmol) at 0° C. The resulting mixture was stirred at that temperature for 3 h. The reaction was complete detected by TLC. After concentration, the residue was purified on an ISCO chromatography to afford compound 55c (666 mg, yield: 91.1%). ESI-MS (m/z): 262.0 [M+1]⁺.

Step 3: To a stirred solution of 55c (666 mg, 2.55 mmol), phthalimide (450 mg, 3.06 mmol) and PPh₃ (1 g, 3.83 mmol) in THF (10 mL) was added DIAD (774 mg, 3.83 mmol) at −10° C. The resulting mixture was stirred at that temperature for 3 h. The reaction was complete detected by TLC. After concentration, the residue was purified on an ISCO chromatography to afford compound 55d (752 mg, yield: 75.6%). ESI-MS (m/z): 391.0 [M−1]⁺.

Step 4: A solution of compound 55d (752 mg, 1.93 mmol). Zn(CN) 2 (680 mg, 5.79 mmol), Pd₂(dba)₃ (89 mg, 0.10 mmol), and 1,1′-Bis(diphenylphosphino) ferrocene (dppf) (105 mg, 0.19 mmol) in DMAc (4 mL) was degassed with N₂ for three times and stirred at 90° C. for 1 h. The reaction was complete detected by TLC. The mixture was partitioned between water and EtOAc. The separated organic phases were concentrated, then purified on an ISCO chromatography to obtain the 55e (503 mg, yield: 90.1%). ESI-MS (m/z): 290.1 [M+1]⁺.

Step 5: To a stirred solution of compound 55e (503 mg, 1.74 mmol) in THF (2 mL) was added NH₂NH₂·H₂O (218 mg, 348 mmol) at room temperature. The resulting mixture was stirred at that temperature for 2 h. The reaction was complete detected by TLC. The reaction mixture was concentrated, then purified on an ISCO chromatography to afford compound 55f (236 mg, yield: 85.2%). ESI-MS (m/z): 160.1 [M+1]⁺.

Step 6: A solution of compound 55f (236 mg, 1.48 mmol), compound 41a (422 mg, 1.48 mmol), Sodium tert-butoxide (426 mg, 4.44 mmol), and RuPhos Pd G3 (126 mg, 0.15 mmol) in toluene (2 mL) was degassed with N₂ for three times and stirred at 100° C. for 2 h. The reaction was complete detected by TLC. After concentration, the mixture was purified on an ISCO chromatography to afford compound 55g (54 mg, yield: 11.5%). ESI-MS (m/z): 317.1 [M+1]⁺.

Step 7: To a stirred solution of compound 55g (54 mg, 0.17 mmol) in a mixture of solvent DMSO (0.3 mL) and ethylene glycol (0.3 mL) was added aq. 50% NaOH (340 mg, 4.25 mmol). The resulting mixture was stirred at 100° C. overnight. The reaction was complete detected by TLC. The mixture was cooled to room temperature and the pH was adjusted to 5 with 1M HCl (aq.). The resulting precipitate was collected by, filtration, and dried to give compound 55h (25 mg, yield: 43.9%). ESI-MS (m/z): 334.1 [M−1]⁺.

Step 8: A solution of compound 55h (25 mg, 0.075 mmol), 3-cyclopropylaniline (12 mg, 0.09 mmol), EDCI (29 mg, 0.15 mmol), and DMAP (1 mg, 0.008 mmol) in DCM (0.1 mL) was stirred at room temperature overnight. The reaction was complete detected by TLC. After concentration, the mixture was purified on an ISCO chromatography to afford compound 55 (12 mg, yield: 35.5%). ESI-MS (m/z): 451.2 [M+1]⁺. ¹H NMR (400 MHz, DMSO-d₆) δ 10.12 (s, 1H), 8.23 (s, 1H), 8.05 (s, 1H), 7.97 (s, 1H), 7.78 (s, 1H), 7.59-7.57 (d, J=8.0 Hz, 1H), 7.56-7.54 (d, J=8.0 Hz, 1H), 7.50 (s, 1H), 7.46-7.45 (d, J=4.0 Hz, 1H), 7.44-7.43 (d, J=4.0 Hz, 1H), 7.33-7.29 (dd, J=8.0, 8.0 Hz, 1H), 7.23-7.19 (dd, J=8.0, 8.0 Hz, 1H), 6.84-6.82 (d, J=8.0 Hz, 1H), 5.64-5.58 (dd, J=14.4, 8.0 Hz, 1H), 3.88 (s, 3H), 3.28-3.22 (m, 1H), 3.11-3.00 (m, 1H), 2.61-2.55 (m, 1H), 1.95-1.86 (m, 2H), 0.99-0.94 (m, 2H), 0.67-0.63 (m, 2H).

### Example 56

### (S)-1-((2-(1-methyl-1H-pyrazol-4-yl) pyridin-4-yl)oxy)-N-(4-(trifluoromethyl)phenyl)-2,3-dihydro-1H-indene-4-carboxamide

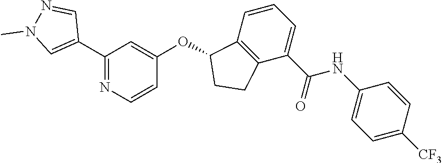 [SMILES: Cn1cc(-c2cc(O[C@H]3CCc4c(C(=O)Nc5ccc(C(F)(F)F)cc5)cccc43)ccn2)cn1]

Compound 56 was prepared by the following steps:

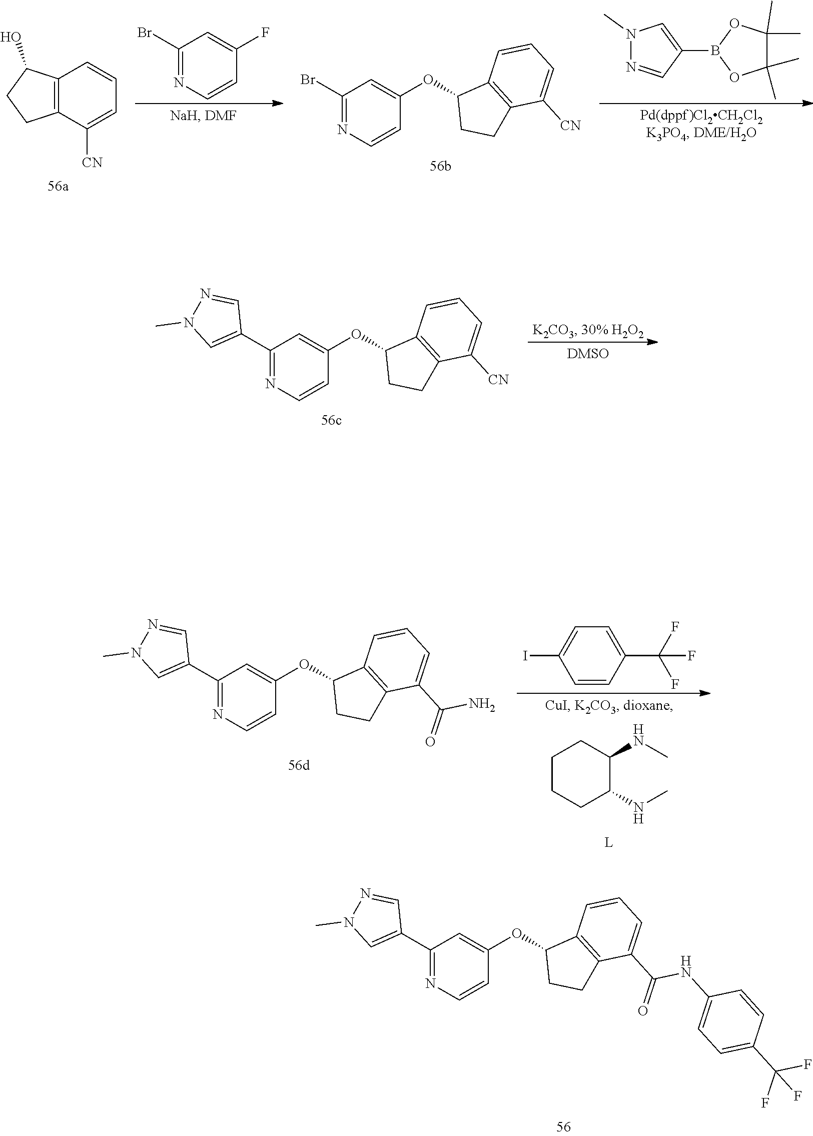

Step 1: To a solution of compound 56a (1.0 g, 6.3 mmol) in dry DMF (7 mL) cooled to 0° C. was added 60% NaH (303 mg, 7.6 mmol), followed by addition of 2-bromo-4-fluoropyridine (1106 mg, 6.3 mmol) after 5 minutes of stirring. The resulting mixture was allowed to warm to room temperature and stirred overnight. The reaction was complete detected by TLC. The reaction was quenched by slow addition of water (30 mL). The resulting mixture was extracted with EtOAc (50 mL*3). The combined organic phases were washed with water (30 mL*2), dried over anhydrous sodium sulfate, and concentrated. The residue was purified by column chromatography to afford compound 56b (929 mg, yield: 46.8%). ESI-MS (m/z): 315.1 [M+H].

Step 2: To the mixture of compound 56b (900 mg, 2.9 mmol), compound 1g (891 mg, 4.3 mmol), K₃PO₄ (1819 mg, 8.6 mmol) and Pd(dppf)Cl₂·CH₂Cl₂ (114 mg, 0.14 mmol) was added 1,2-dimethoxyethane (4.0 mL) and H₂O (1.5 mL). The resulting mixture was degassed with N₂ for three times and refluxed at 90° C. for 1 h. The reaction was complete detected by TLC. The reaction mixture was diluted with DCM (200 mL), washed with water (50 mL*2). The organic phase was dried over anhydrous sodium sulfate and concentrated to dryness. The residue was purified by gel column chromatography to afford compound 56c (824 mg, yield: 91%). ESI-MS (m/z): 317.1 [M+H]⁺.

Step 3: To a stirred solution of compound 56c (824 mg, 2.6 mmol) and K₂CO₃ in DMSO (9.6 mL) was slowly added 30% H₂O₂ (4.8 mL). The resulting mixture was stirred at room temperature for 1 h. The reaction was complete detected by TLC. The mixture was diluted with water (50 mL), extracted with EtOAc (100 mL*4). The combined organic phases were washed H₂O (30 mL*2), dried over anhydrous sodium sulfate, and concentrated to dryness. The residue was purified by column chromatography to afford compound 56d (483 mg, yield: 55.6%). ESI-MS (m/z): 335.1 [M+H]⁺.

Step 4: To the mixture of compound 56d (100 mg, 0.3 mmol), 4-Iodobenzotrifluoride (98 mg, 0.36 mmol), K₃PO₄ (127 mg, 0.6 mmol), CuI (6 mg, 0.03 mmol) and (1R, 2R)—N,N′-dimethyl-1,2-cyclohexanediamine (4.3 mg, 0.03 mmol) was added 1,4-dioxane (1 mL). The mixture was stirred at 80° C. for 3 h under N₂. The reaction was complete detected by TLC. The reaction mixture was diluted with EtOAc (50 mL), and washed sequentially with Ammonium hydroxide (10 mL*2) and water (10 mL). The organic phase was dried over anhydrous sodium sulfate, and concentrated to dryness. The residue was purified by column chromatography to afford compound 56 (55 mg, yield: 38.3%) as a pale yellow solid. ESI-MS (m/z): 479.1 [M+H]⁺. ¹H NMR (400 MHz, DMSO-d₆) δ 10.64 (s, 1H), 8.35 (d, J=5.8 Hz, 1H), 8.29 (s, 1H), 8.02 (s, 1H), 7.99 (d, J=8.5 Hz, 2H), 7.80-7.71 (m, 3H), 7.66 (d, J=7.5 Hz, 1H), 7.45 (t, J=7.6 Hz, 1H), 7.33 (d, J=2.4 Hz, 1H), 6.91 (dd, J=5.8, 2.4 Hz, 1H), 6.12 (dd, J=6.6, 3.6 Hz, 1H), 3.88 (s, 3H), 3.35-3.26 (m, 1H), 3.17 (d, J=5.2 Hz, 1H), 2.72-2.60 (m, 1H), 2.16-2.03 (m, 1H).

### Example 57

### (S)—N-(3-cyclopropylphenyl)-1-((2-(1-methyl-1H-pyrazol-4-yl) pyridin-4-yl)amino)-2,3-dihydro-1H-indene-4-carboxamide

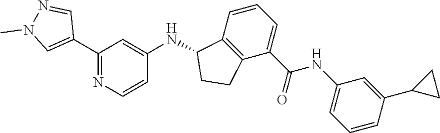 [SMILES: Cn1cc(-c2cc(N[C@H]3CCc4c(C(=O)Nc5cccc(C6CC6)c5)cccc43)ccn2)cn1]

Compound 57 was prepared by the following steps:

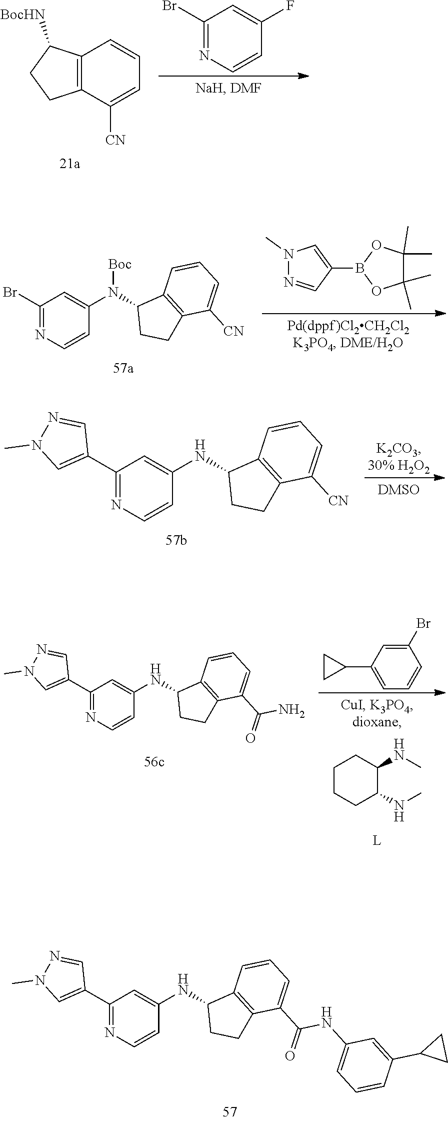

The preparation of compound 57 refers to the synthesis of compound 56 in example 56.

ESI-MS (m/z): 450.2 [M+H]⁺. ¹H NMR (400 MHz, DMSO-d₆) δ 10.14 (s, 1H), 8.23 (s, 1H), 8.02 (d, J=6.0 Hz, 1H), 7.95 (s, 1H), 7.63 (d, J=7.4 Hz, 1H), 7.54 (d, J=8.1 Hz, 1H), 7.49 (d, J=2.0 Hz, 1H), 7.43 (d, J=7.4 Hz, 1H), 7.36 (t, J=7.5 Hz, 1H), 7.21 (t, J=7.9 Hz, 1H), 7.03 (s, 1H), 6.83 (d, J=7.8 Hz, 1H), 6.61-6.56 (m, 1H), 5.26 (q, J=7.7 Hz, 1H), 3.89 (s, 3H), 3.31-3.18 (m, 1H), 3.12-2.99 (m, 2H), 1.94-1.82 (m, 2H), 0.99-0.94 (m, 2H), 0.69-0.62 (m, 2H).

### Example 58

### (S)-5-(1-methyl-1H-pyrazol-4-yl)-N-(4-(((4-(trifluoromethyl)phenyl)amino)methyl)-2,3-dihydro-1H-inden-1-yl) pyridin-3-amine

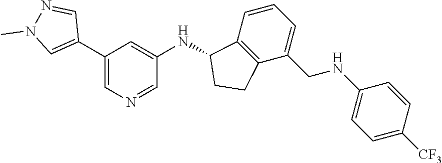 [SMILES: Cn1cc(-c2cncc(N[C@H]3CCc4c(CNc5ccc(C(F)(F)F)cc5)cccc43)c2)cn1]

Compound 58 was prepared by the following steps:

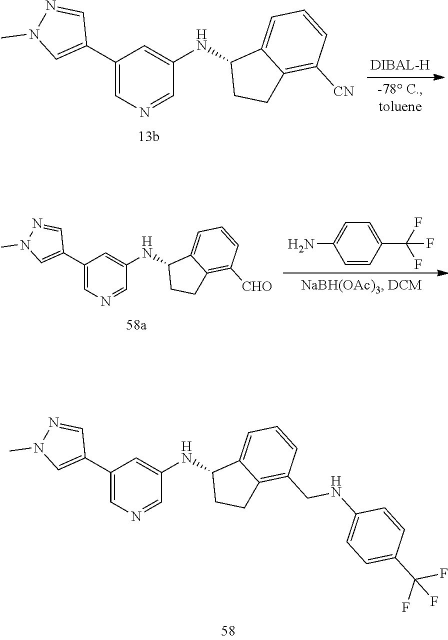 [SMILES: Cn1cc(-c2cncc(N[C@H]3CCc4c(C#N)cccc43)c2)cn1 ; Cn1cc(-c2cncc(N[C@H]3CCc4c(C=O)cccc43)c2)cn1 ; Nc1ccc(C(F)(F)F)cc1 ; Cn1cc(-c2cncc(N[C@H]3CCc4c(CNc5ccc(C(F)(F)F)cc5)cccc43)c2)cn1]

Step 1: A solution of compound 13b (460 mg, 1.5 mmol) in toluene (5 mL) was degassed with N₂ for three times, then added DIBAL-H (1N in toluene, 5 mL, 5.0 mmol) at −78° C. The resulting mixture was stirred at that temperature for 2 h. The reaction was complete detected by TLC. The reaction mixture was quenched by addition of water (10 mL) dropwise. The separated organic phase was dried, and concentrated to give compound 58a (552 mg, yield: 87%). ESI-MS (M/S): 319.2 [M+H]⁺.

Step 2: To a stirred solution of compound 58a (159 mg, 0.5 mmol) in CH₂Cl₂ (1 mL) was added 4-aminobenzotrifluoride (242 mg, 1.5 mmol) and NaBH(OAc)₃ (318 mg, 1.5 mmol). The resulting mixture was stirred at room temperature for 3 h. The reaction was complete detected by TLC. The reaction mixture was washed with saturated aq. NaHCO₃ twice. The separated organic phase was, dried, and concentrated. The residue was purified by column chromatography to afford compound 58 (178 mg, yield: 77%). ESI-MS (M/S): 464.0 [M+H]. ¹H NMR (400 MHz, DMSO-d₆) δ 8.16 (s, 1H), 8.03 (d, J=1.8 Hz, 1H), 7.93 (d, J=2.6 Hz, 1H), 7.86 (d, J=0.7 Hz, 1H), 7.36 (d, J=8.6 Hz, 2H), 7.27-7.12 (m, 4H), 6.94 (t, J=5.9 Hz, 1H), 6.69 (d, J=8.5 Hz, 2H), 6.21 (d, J=8.4 Hz, 1H), 5.10 (q, J=7.6 Hz, 1H), 4.33 (d, J=5.8 Hz, 2H), 3.87 (s, 3H), 3.06 (ddd, J=16.1, 8.8, 3.4 Hz, 1H), 2.85 (dt, J=16.1, 8.1 Hz, 1H), 2.62 (dt, J=12.0, 5.7 Hz, 1H), 1.81 (dt, J=12.5, 8.1 Hz, 1H).

### Example 59

The preparation of compounds 59a-59f refers to the synthesis of compound 58 in example 58.

TABLE 16

| | | MS | |
|---|---|---|---|
| | | [M + | |
| Cpds | Structure | 1]⁺ | ¹H NMR |
| 59a | 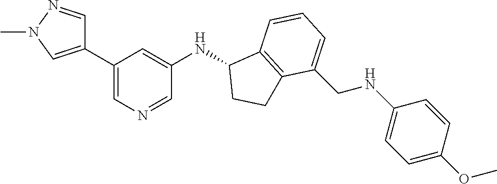 | 426.2 | ¹H NMR (62 MHz, acetone-d₆) δ 8.22-7.77 (m, 4H), 7.17 (s, 4H), 6.85-6.36 (m, 4H), 6.17 (d, J = 8.3 Hz, 1H), 5.80-5.46 (m, 1H), 5.24-4.91 (m, 1H), 4.19 (d, J = 5.2 Hz, 2H), 3.86 (s, 3H), 3.62 (s, 3H), 3.13-2.55 (m, 3H), 2.07-1.65 (m, 1H). |
| 59b | 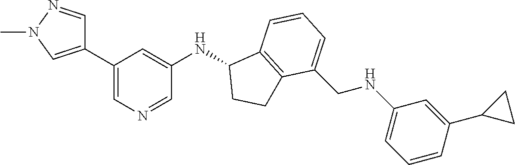 [SMILES: Cn1cc(-c2cncc(N[C@H]3CCc4c(CNc5cccc(C6CC6)c5)cccc43)c2)cn1] | 436.2 | ¹H NMR (400 MHz, DMSO-d₆) δ 8.20-7.84 (m, 4H), 7.26-7.13 (m, 4H), 6.90 (t, J = 7.9 Hz, 1H), 6.32 (d, J = 7.3 Hz, 2H), 6.21 (dd, J = 12.5, 8.0 Hz, 2H), 6.01 (t, J = 6.0 Hz, 1H), 5.09 (q, J = 7.7 Hz, 1H), 4.23 (d, J = 5.9 Hz, 2H), 3.86 (s, 3H), 3.11-3.01 (m, 1H), 2.85 (dt, J = 16.2, 8.0 Hz, 1H), 2.60 (d, J = 9.3 Hz, 1H), 1.77 (ddd, J = 26.9, 11.5, 6.2 Hz, 2H), 0.85 (d, J = 7.7 Hz, 2H), 0.55 (d, J = 5.2 Hz, 2H). |
| 59c | 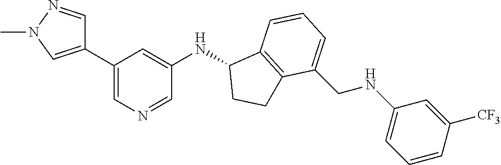 [SMILES: Cn1cc(-c2cncc(N[C@H]3CCc4c(CNc5cccc(C(F)(F)F)c5)cccc43)c2)cn1] | 464.2 | ¹H NMR (400 MHz, DMSO-d₆) δ 8.16 (s, 1H), 8.03 (s, 1H), 7.93 (s, 1H), 7.86 (s, 1H), 7.22 (dq, J = 17.9, 8.3 Hz, 5H), 6.90 (s, 1H), 6.81 (t, J = 6.8 Hz, 2H), 6.67 (t, J = 5.9 Hz, 1H), 6.20 (d, J = 8.4 Hz, 1H), 5.10 (q, J = 7.7 Hz, 1H), 4.31 (d, J = 5.8 Hz, 2H), 3.87 (s, 3H), 3.12-3.00 (m, 1H), 2.86 (dt, J = 16.2, 8.2 Hz, 1H), 2.61 (d, J = 10.1 Hz, 1H), 1.82 (dq, J = 16.2, 8.6 Hz, 1H). |
| 59d | 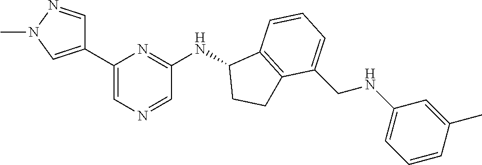 | 411.2 | ¹H NMR (62 MHz, acetone-d₆) δ 8.13 (s, 1H), 8.06 (s, 1H), 7.96 (s, 1H), 7.83 (s, 1H), 7.44- 6.75 (m, 4H), 6.50 (s, 2H), 6.37 (s, 1H), 5.93- 5.40 (m, 1H), 4.32 (s, 2H), 3.91 (s, 3H), 3.26- 2.85 (m, 2H), 2.80-2.46 (m, 1H), 2.19 (s, 3H), 1.99-1.80 (m, 1H). |
| 59e | 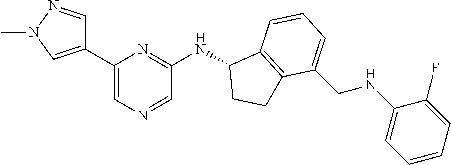 [SMILES: Cn1cc(-c2cncc(N[C@H]3CCc4c(CNc5ccccc5F)cccc43)n2)cn1] | 415.2 | ¹H NMR (400 MHz, DMSO-d₆) δ 8.23 (s, 1H), 7.99 (d, J = 22.5 Hz, 2H), 7.75 (s, 1H), 7.38 (d, J = 7.8 Hz, 1H), 7.18-7.08 (m, 3H), 7.06-6.97 (m, 1H), 6.88 (t, J = 7.5 Hz, 1H), 6.54 (t, J = 8.8 Hz, 2H), 6.07 (s, 1H), 5.60 (d, J = 7.5 Hz, 1H), 4.34 (d, J = 5.7 Hz, 2H), 3.87 (s, 3H), 3.18-3.03 (m, 1H), 2.90 (d, J = 8.4 Hz, 1H), 2.60 (d, J = 10.9 Hz, 1H), 1.92-1.83 (m, 1H). |
| 59f | 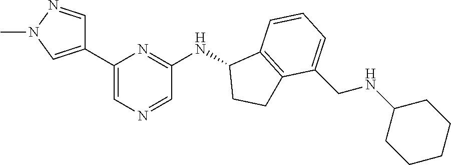 [SMILES: Cn1cc(-c2cncc(N[C@H]3CCc4c(CNC5CCCCC5)cccc43)n2)cn1] | 403.3 | ¹H NMR (400 MHz, DMSO-d₆) δ 8.22 (s, 1H), 8.03 (s, 1H), 7.96 (s, 1H), 7.77 (s, 1H), 7.42-7.13 (m, 4H), 5.61-5.56 (m, 1H), 3.90 (s, 2H), 3.88 (s, 3H), 3.11-3.04 (m, 1H), 2.91-2.83 (m, 1H), 2.71-2.68 (m, 1H), 2.62- 2.56 (m, 1H), 2.09-1.83 (m, 5H), 1.73 (s, 2H), 1.61-1.58 (m, 1H), 1.26-1.19 (m, 4H). |

### Example 60

The preparation of compounds 60a-60e refers to step 2 (reductive amination) of example 58.

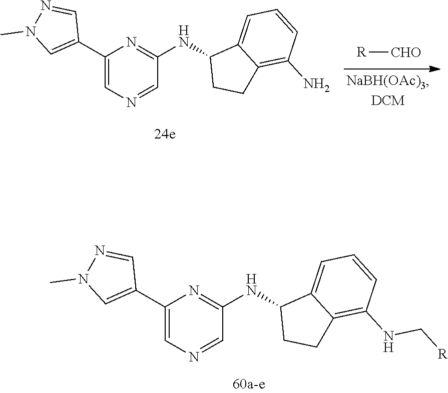

TABLE 17

| | | MS | |
|---|---|---|---|
| | | [M + | |
| Cpds | Structure | 1]⁺ | ¹H NMR |
| 60a | 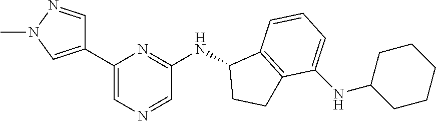 [SMILES: Cn1cc(-c2cncc(N[C@H]3CCc4c(NC5CCCCC5)cccc43)n2)cn1] | 389.2 | ¹H NMR (62 MHz, acetone-d₆) δ 8.13-7.81 (m, 4H), 7.23-6.11 (m, 4H), 5.62 (s, 1H), 3.93 (s, 3H), 3.41 (s, 1H), 2.69-2.51 (m, 2H), 1.88- 1.01 (m, 13H). |
| 60b | 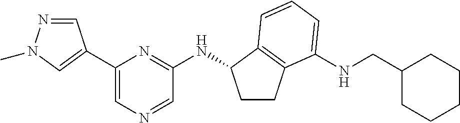 [SMILES: Cn1cc(-c2cncc(N[C@H]3CCc4c(NCC5CCCCC5)cccc43)n2)cn1] | 403.1 | ¹H NMR (62 MHz, acetone-d₆) δ 8.20-7.72 (m, 4H), 7.30-6.06 (m, 4H), 5.90-5.32 (m, 1H), 3.92 (s, 3H), 3.12-2.49 (m, 5H), 1.78-1.03 (m, 13H). |
| 60c | 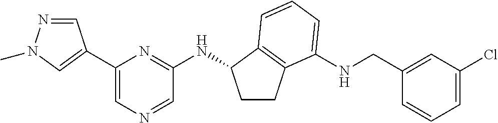 [SMILES: Cn1cc(-c2cncc(N[C@H]3CCc4c(NCc5cccc(Cl)c5)cccc43)n2)cn1] | 431.2 | ¹H NMR (62 MHz, acetone-d₆) δ 8.15-7.78 (m, 4H), 7.39 (d, J = 6.6 Hz, 4H), 7.06-6.24 (m, 4H), 5.78-5.15 (m, 2H), 4.60-4.39 (m, 2H), 3.92 (s, 3H), 3.00-2.59 (m, 4H). |
| 60d | 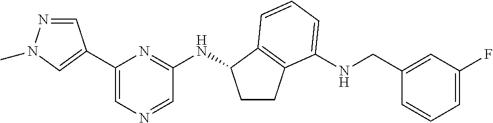 [SMILES: Cn1cc(-c2cncc(N[C@H]3CCc4c(NCc5cccc(F)c5)cccc43)n2)cn1] | 415.2 | ¹H NMR (62 MHz, acetone-d₆) δ 8.21 (s, 1H), 7.97 (d, J = 2.4 Hz, 2H), 7.72 (s, 1H), 7.42-5.38 (m, 10H), 4.37 (d, J = 6.1 Hz, 2H), 3.87 (s, 3H), 2.73 (s, 3H), 2.08-1.69 (m, 1H). |
| 60e | 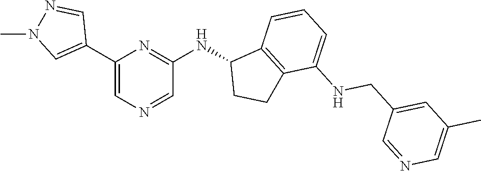 | 412.2 | ¹H NMR (62 MHz, acetone-d₆) δ 8.36 (d, J = 8.1 Hz, 2H), 8.23-7.92 (m, 3H), 7.81 (s, 1H), 7.60 (s, 1H), 7.15-6.18 (m, 4H), 5.87-5.06 (m, 2H), 4.45 (d, J = 5.8 Hz, 2H), 3.92 (s, 3H), 2.98-2.57 (m, 3H), 2.30 (s, 3H), 1.82 (s, 1H). |

### Example 61

### (1S)—N⁴-(1-(3-chlorophenyl)-2,2,2-trifluoroethyl)-N₁-(6-(1-methyl-1H-pyrazol-4-yl) pyrazin-2-yl)-2,3-dihydro-1H-indene-1,4-diamine

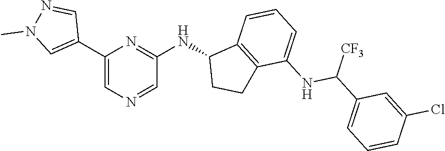 [SMILES: Cn1cc(-c2cncc(N[C@H]3CCc4c(NC(c5cccc(Cl)c5)C(F)(F)F)cccc43)n2)cn1]

Compound 61 was prepared by the following steps:

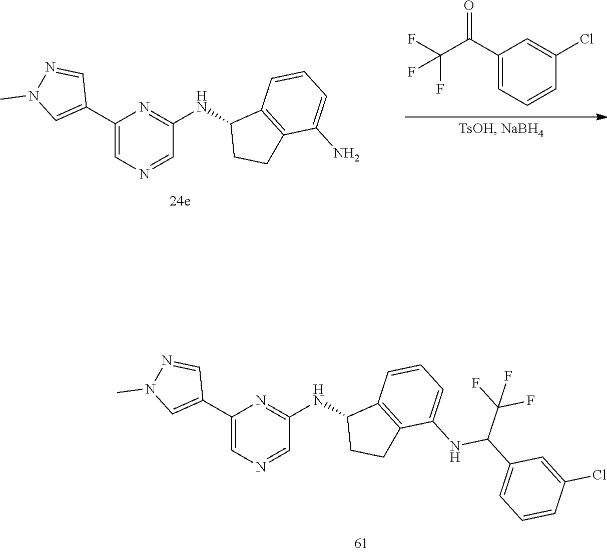 [SMILES: Cn1cc(-c2cncc(N[C@H]3CCc4c(N)cccc43)n2)cn1 ; O=C(c1cccc(Cl)c1)C(F)(F)F ; Cn1cc(-c2cncc(N[C@H]3CCc4c(NC(c5cccc(Cl)c5)C(F)(F)F)cccc43)n2)cn1]

The mixture of 24e (150 mg, 0.5 mmol), 1-(3-Chlorophenyl)-2,2,2-trifluoroethanone and p-toluenesulfonic acid (9 mg, 0.05 mmol) was heated to 100° C. and stirred for 12 h. After natural cooling to room temperature, MeOH (5 mL) and NaBH₄ (190 mg, 5.0 mmol) were added to the above mixture in turn. The resulting mixture was stirred at room temperature for 30 min. TLC analysis showed the reaction was complete. EtOAc (15 mL) was added to the reaction mixture. The resulting mixture was washed with water (15 mL*3), dried over Na₂SO₄, and concentrated to dryness. The residue was purified by chromatography to give product 61 (17 mg, yield: 7%). ESI-MS (m/z): 499.5 [M+H]⁺. ¹H NMR (62 MHz, acetone-d₆) δ 8.24-7.89 (m, 3H), 7.88-7.58 (m, 3H), 7.58-7.34 (m, 2H), 7.16-6.55 (m, 3H), 6.44-6.31 (m, 1H), 5.83-5.52 (m, 3H), 3.92 (s, 3H), 3.61-2.90 (m, 2H), 2.74-2.43 (m, 1H), 1.29-1.20 (m, 1H).

### Example 62

### cyclohexyl(S)-1-((6-(1-methyl-1H-pyrazol-4-yl) pyrazin-2-yl)amino)-2,3-dihydro-1H-indene-4-carboxylate

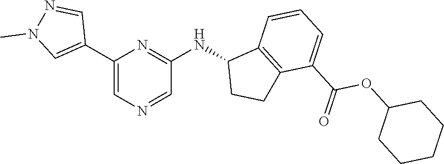 [SMILES: Cn1cc(-c2cncc(N[C@H]3CCc4c(C(=O)OC5CCCCC5)cccc43)n2)cn1]

Compound 62 was prepared by the following steps:

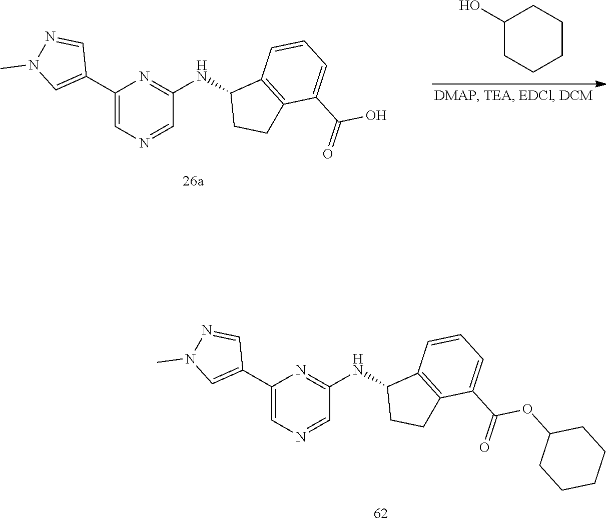 [SMILES: Cn1cc(-c2cncc(N[C@H]3CCc4c(C(=O)O)cccc43)n2)cn1 ; OC1CCCCC1 ; Cn1cc(-c2cncc(N[C@H]3CCc4c(C(=O)OC5CCCCC5)cccc43)n2)cn1]

To a solution of 26a (33 mg, 0.1 mmol), cyclohexanol (20 mg, 0.2 mmol), DMAP (1 mg, 0.01 mmol) and Et₃N (20 mg, 0.2 mmol) in CH₂Cl₂ (0.5 mL) was added EDCI (38 mg, 0.2 mmol). The resulting mixture was stirred at room temperature until TLC analysis showed the reaction was complete. The mixture was concentrated to dryness and the residue was purified by chromatography to give product 62 (37 mg, yield: 88%). ESI-MS (m/z): 417.2 [M+H]⁺. ¹H NMR (62 MHz, acetone-d₆) δ 7.91 (dd, J=12.3, 7.1 Hz, 5H), 7.67-7.10 (m, 3H), 5.58 (q, J=7.6 Hz, 1H), 4.82 (d, J=8.2 Hz, 2H), 3.95 (s, 3H), 3.69-3.12 (m, 2H), 3.09-2.00 (m, 2H), 1.95 (s, 2H), 1.55 (s, 8H).

### Example 63

### (S)—N¹-(5-(1-methyl-1H-pyrazol-4-yl) pyridin-3-yl)-N⁴-(3-phenyloxetan-3-yl)-2,3-dihydro-1H-indene-1,4-diamine

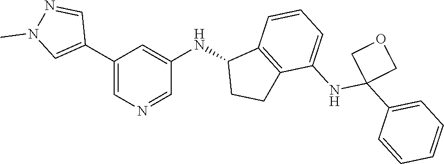 [SMILES: Cn1cc(-c2cncc(N[C@H]3CCc4c(NC5(c6ccccc6)COC5)cccc43)c2)cn1]

Compound 63 was prepared by the following steps:

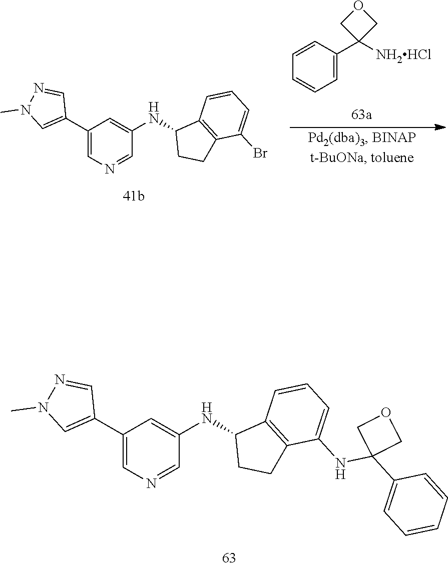

To a solution of 41b (74 mg, 0.2 mmol) in DMSO (0.2 mL) was added 63a (74 mg, 0.4 mmol), K₂CO₃ (260 mg, 0.8 mmol), BINAP (25 mg, 0.04 mmol) and Pd₂(dba)₃ (9 mg, 0.01 mmol). The resulting mixture was degassed with N₂ for three times, then heated to 90° C. and stirred until TLC analysis showed the reaction was complete. After natural cooling to room temperature. EtOAc (5 mL) was added to the reaction mixture. The mixture was washed with water (5 mL*3), dried over Na₂SO₄, and concentrated to dryness. The residue was purified by chromatography to give product 63 (11 mg, yield: 13%). ESI-MS (m/z): 438.2 [M+H]⁺. ¹H NMR (400 MHz, DMSO-d₆) δ 8.15 (s, 1H), 8.01 (s, 1H), 7.97-7.87 (m, 1H), 7.85 (s, 1H), 7.62 (s, 1H), 7.60 (s, 1H), 7.40-7.37 (m, 2H), 7.29-7.25 (m, 1H), 7.21 (s, 1H), 6.76 (t, J=7.8 Hz, 1H), 6.53 (d, J=7.5 Hz, 1H), 6.39 (s, 1H), 6.17 (d, J=8.5 Hz, 1H), 5.44 (d, J=8.0 Hz, 1H), 5.09-5.03 (m, 1H), 4.92-7.91 (m, 2H), 4.86-7.84 (m, 2H), 3.87 (s, 3H), 3.03-2.97 (m, 1H), 2.82-2.74 (m, 1H), 2.62-2.58 (m, 1H), 1.90-1.76 (m, 1H).

### Example 64

### (S)-3,3-dimethyl-N-(1-((6-(1-methyl-1H-pyrazol-4-yl) pyrazin-2-yl)amino)-2,3-dihydro-1H-inden-4-yl)-2-oxopyrrolidine-1-carboxamide

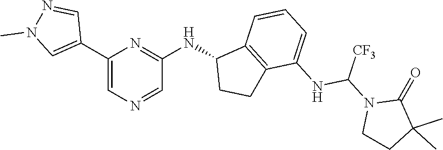

Compound 64 was prepared by the following steps:

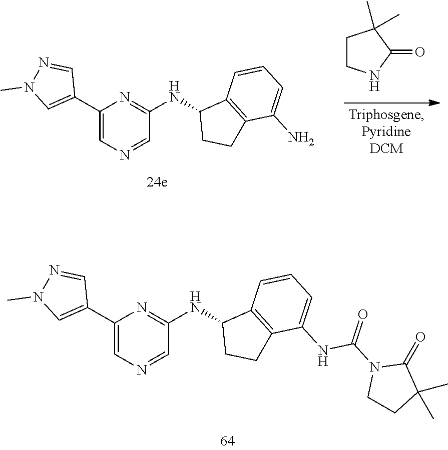

To a solution of triphosgene (88 mg, 0.296 mmol) in CH₂Cl₂ (1 mL) was added dropwise a solution of 3,3-Dimethyl-2-oxopyrrolidine (100 mg, 0.885 mmol) and pyridine (209 mg, 0.267 mmol) in CH₂Cl₂ (1 mL) at 0° C., followed by addition dropwise of a solution of pyridine (20 mg) and 24e (75 mg, 0.245 mmol) in CH₂Cl₂ (2 mL) after stirring for 0.5 h. The resulting mixture was stirred for 2 h, then washed with water twice. The organic layer was dried and concentrated. The residue was purified by chromatography to give product 64 (57 mg, yield: 52%). ESI-MS (M/S): 446.1 [M+H]⁺. ¹H NMR (62 MHz, acetone-d₆) δ 10.65 (s, 1H), 8.34-7.66 (m, 5H), 7.15 (d, J=6.6 Hz, 2H), 6.50 (d, J=8.1 Hz, 1H), 5.92-5.54 (m, 1H), 3.92 (s, 3H), 3.83-2.93 (m, 4H), 2.75-2.22 (m, 2H), 1.26 (s, 6H), 1.15-0.87 (m, 2H).

### Example 65

Compounds 65a-65e were prepared by the following steps:

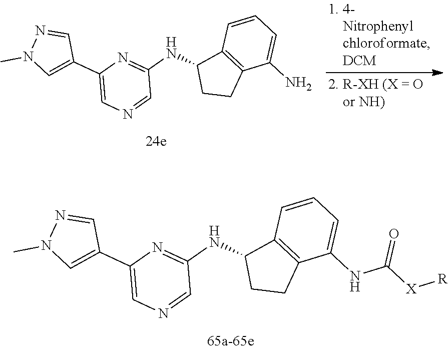

To a stirred solution of 24e (61 mg, 0.2 mmol) in CH₂Cl₂ (1.0 mL) cooled to −20° C. was added p-Nitrophenyl chloroformate (40 mg, 0.2 mmol) under N₂. After stirring at −20° C. for 30 min, amine or alcohol was added to the above mixture. The resulting mixture was continued to stir for 15 min at room temperature. TLC was used to monitored the reaction. After the reaction was complete, the reaction mixture was concentrated to dryness and the residue was purified by chromatography to give the desired product.

TABLE 18

| | | MS | |
|---|---|---|---|
| Cpds | Structure | [M + 1]⁺ | ¹H NMR |
| 65a | 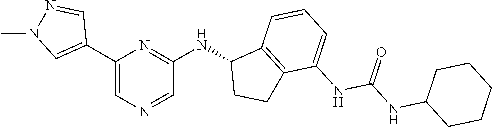 [SMILES: Cn1cc(-c2cncc(N[C@H]3CCc4c(NC(=O)NC5CCCCC5)cccc43)n2)cn1] | 432.2 | ¹H NMR (400 MHz, DMSO-d₆) δ 8.22 (s, 1H), 7.99 (d, J = 20.8 Hz, 2H), 7.86 (d, J = 8.1 Hz, 1H), 7.73 (d, J = 11.8 Hz, 2H), 7.35 (d, J = 8.0 Hz, 1H), 7.05 (t, J = 7.8 Hz, 1H), 6.85 (d, J = 7.4 Hz, 1H), 6.50 (d, J = 7.7 Hz, 1H), 5.57 (q, J = 7.6 Hz, 1H), 3.88 (s, 3H), 3.48 (d, J = 9.3 Hz, 1H), 2.88 (s, 1H), 2.71 (dt, J = 15.4, |
| | | | 7.7 Hz, 1H), 2.61-2.51 (m, 1H), 1.85 |
| | | | (dd, J = 18.7, 12.3 Hz, 3H), 1.67 (d, |
| | | | J = 12.8 Hz, 2H), 1.55 (d, J = 12.2 Hz, |
| | | | 1H), 1.33 (q, J = 11.9 Hz, 2H), 1.17 (q, |
| | | | J = 11.7, 11.3 Hz, 3H). |
| 65b | 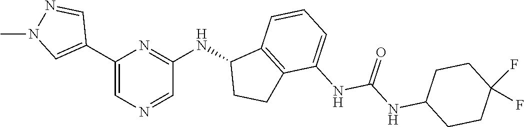 [SMILES: Cn1cc(-c2cncc(N[C@H]3CCc4c(NC(=O)NC5CCC(F)(F)CC5)cccc43)n2)cn1] | 468.1 | ¹H NMR (62 MHz, acetone-d₆) δ 8.21 (s, 1H), 8.09-7.60 (m, 5H), 7.44-6.51 (m, 4H), 5.48 (m, 1H), 3.87 (s, 3H), 3.76- 3.47 (m, 1H), 2.82 (m, 2H), 2.35-0.99 (m, 10H). |
| 65c | 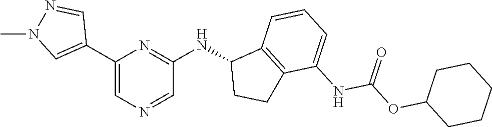 [SMILES: Cn1cc(-c2cncc(N[C@H]3CCc4c(NC(=O)OC5CCCCC5)cccc43)n2)cn1] | 433.1 | ¹H NMR (62 MHz, acetone-d₆) δ 8.14 (s, 1H), 8.06 (s, 1H), 7.96 (s, 1H), 7.82 (s, 1H), 7.77-7.56 (m, 1H), 7.14 (d, J = 3.5 Hz, 1H), 7.07 (s, 1H), 6.46 (d, J = 8.0 Hz, 1H), 5.93-5.46 (m, 1H), 5.01-4.34 (m, 1H), 3.92 (s, 3H), 3.25-2.86 (m, 2H), 2.75-2.41 (m, 1H), 1.95-1.11 (m, 11H). |
| 65d | 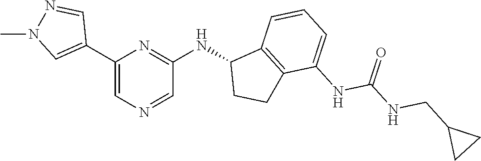 [SMILES: Cn1cc(-c2cncc(N[C@H]3CCc4c(NC(=O)NCC5CC5)cccc43)n2)cn1] | 404.2 | ¹H NMR (400 MHz, DMSO-d₆) δ 8.22 (s, 1H), 7.99 (d, J = 21.0 Hz, 2H), 7.89-7.81 (m, 2H), 7.75 (s, 1H), 7.35 (d, J = 7.9 Hz, 1H), 7.06 (t, J = 7.8 Hz, 1H), 6.86 (d, J = 7.4 Hz, 1H), 6.61 (s, 1H), 5.57 (q, J = 7.6 Hz, 1H), 3.88 (s, 3H), 3.03-2.85 (m, 3H), 2.79-2.56 (m, 2H), 1.93-1.83 (m, 1H), 0.95 (td, J = 7.3, 3.1 Hz, 1H), 0.46 (t, J = 6.5 Hz, 2H), 0.19 (d, J = 4.9 Hz, 2H). |
| 65e | 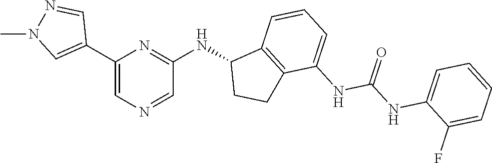 [SMILES: Cn1cc(-c2cncc(N[C@H]3CCc4c(NC(=O)Nc5ccccc5F)cccc43)n2)cn1] | 444.1 | ¹H NMR (400 MHz, DMSO-d₆) δ 8.98 (s, 1H), 8.57 (s, 1H), 8.22 (d, J = 6.2 Hz, 2H), 8.00 (d, J = 22.7 Hz, 2H), 7.89 (d, J = 8.1 Hz, 1H), 7.76 (s, 1H), 7.39 (d, J = 8.0 Hz, 1H), 7.26 (dd, J = 11.6, 8.3 Hz, 1H), 7.15 (td, J = 7.7, 3.3 Hz, 2H), 6.99 (dd, J = 19.7, 7.1 Hz, 2H), 5.61 (q, J = 7.6 Hz, 1H), 3.88 (s, 3H), 3.08-2.92 (m, 1H), 2.81 (dt, J = 15.8, 8.0 Hz, 1H), 2.67-2.56 (m, 1H), 1.92 (dq, J = 15.7, 8.2 Hz, 1H). |

### Example 66

### (S)—N⁴-(5-(tert-butyl) isoxazol-3-yl)-N₁-(6-(1-methyl-1H-pyrazol-4-yl) pyrazin-2-yl)-2,3-dihydro-1H-indene-1,4-diamine

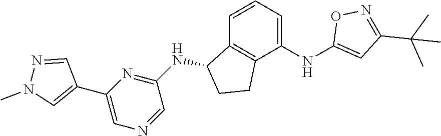 [SMILES: Cn1cc(-c2cncc(N[C@H]3CCc4c(Nc5cc(C(C)(C)C)no5)cccc43)n2)cn1]

Compound 66 was prepared by the following steps:

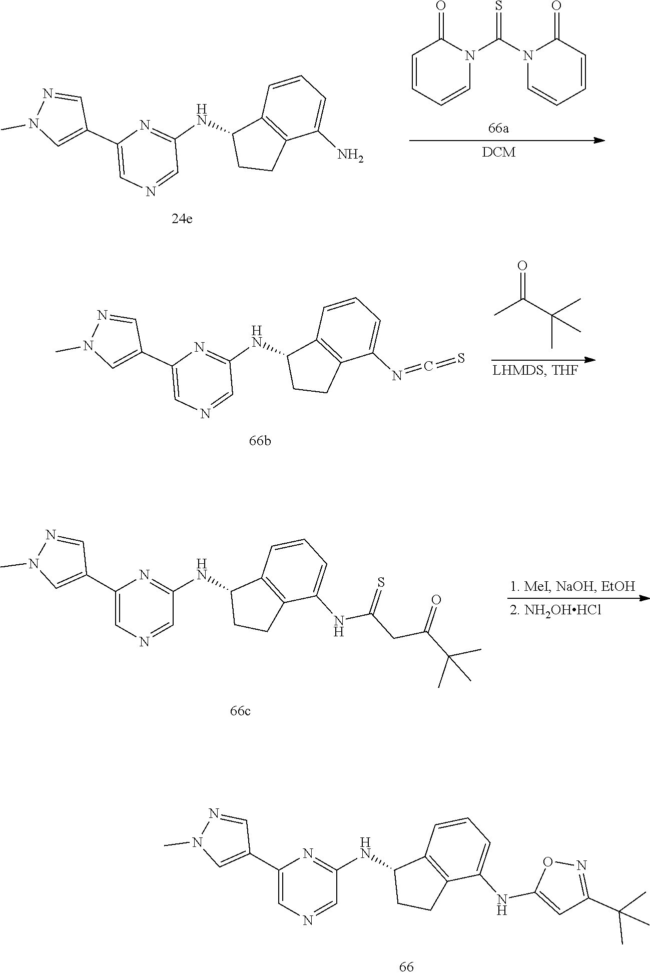

Step 1: To a solution of 24e (153 mg, 0.5 mmol) in CH₂Cl₂ (1 mL) was added 66a (116 mg, 0.5 mmol). The resulting mixture was stirred at room temperature for 1 h. Water (30 mL) was added to the reaction mixture and the mixture was extracted with CH₂Cl₂ (30 mL*3). The combined organic layers were washed with brine (30 mL), dried over Na₂SO₄ and concentrated to dryness to give product 66b (170 mg, 97.6%). ESI-MS (m/z): 349.2 [M+H]⁺.

Step 2: To a stirred solution of 3,3-Dimethyl-2-butanone (75 mg, 0.75 mmol) in TH F (0.5 mL) cooled to 0° C. was added LiHMDS (1 M in THE, 0.75 mL), followed by addition of a solution of 66b (170 mg, 0.49 mmol) in THF (0.5 mL) after 1 h. The resulting mixture was stirred for another 4 h. The reaction was quenched by the addition of saturated aqueous NH₄Cl (10 mL) and stirred for 30 min. The mixture was extracted with EtOAc (10 mL*3). The combined organic layers were dried over Na₂SO₄ filtered and concentrated. The residue was purified by silica gel chromatography to give product 66c as a yellow solid (84 mg, 38.4%). ESI-MS (m/z): 449.3 [M+H]⁺.

Step 3: To a stirred solution of 66c (80 mg, 0.18 mmol) in EtOH (2 mL) was added NaOH (72 mg, 1.8 mmol). The resulting mixture was cooled to −78° C., then Mel (26 mg, 0.18 mmol) was added. After the mixture was stirred for 2 h, hydroxylamine hydrochloride (25 mg, 0.36 mmol) was added to the above mixture. The reaction mixture was heated to 80° C. and stirred for 1 h. After cooling to room temperature, water (20 mL) was added and the mixture was extracted with EtOAc (20 mL*3). The combined organic layers were dried over Na₂SO₄, filtered and concentrated. The residue was purified by silica gel chromatography to give product 66 (32 mg, 41.4%). ESI-MS (m/z): 430.3 [M+H]⁺. ¹H NMR (62 MHz, CDCl₃) δ 8.13-7.76 (m, 2H), 7.41-6.91 (m, 4H), 6.38 (s, 1H), 5.79-5.32 (m, 1H), 5.28-4.93 (m, 1H), 3.95 (s, 3H), 3.14-1.77 (m, 4H), 1.29 (s, 9H).

### Example 67

### (S)—N-(1-((2-amino-5-(1-methyl-1H-pyrazol-4-yl) pyridin-3-yl)oxy)-2,3-dihydro-1H-inden-4-yl)-5-(tert-butyl) isoxazol-3-amine

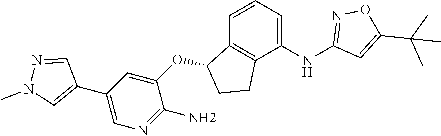 [SMILES: Cn1cc(-c2cnc(N)c(O[C@H]3CCc4c(Nc5cc(C(C)(C)C)on5)cccc43)c2)cn1]

The preparation of compound 67 refers to the synthesis of compound 66 in example 66.

ESI-MS (M/S): 430.10 [M+H]⁺. ¹H NMR (62 MHz, acetone-d₆) δ 8.44 (s, 1H), 8.28-7.86 (m, 4H), 7.75-7.34 (m, 2H), 7.32-6.92 (m, 2H), 6.14-5.88 (m, 2H), 3.94 (s, 3H), 3.21-1.81 (m, 4H), 1.33 (s, 9H).

### Example 68

### (S)—N-(1-((2-amino-5-(1-methyl-1H-pyrazol-4-yl) pyridin-3-yl)oxy)-2,3-dihydro-1H-inden-4-yl)-5-(tert-butyl) isoxazol-3-amine

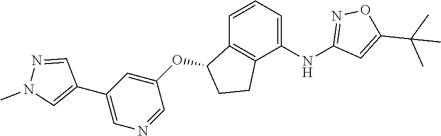 [SMILES: Cn1cc(-c2cncc(O[C@H]3CCc4c(Nc5cc(C(C)(C)C)on5)cccc43)c2)cn1]

The preparation of compound 68 refers to the synthesis of compound 66 in example 66.

ESI-MS (M/S): 445.2 [M+H]⁺. ¹H NMR (62 MHz, acetone-d₆) δ 8.14-7.78 (m, 3H), 7.72 (s, 1H), 7.67-7.34 (m, 2H), 7.33-6.91 (m, 2H), 6.09-5.78 (m, 2H), 5.19-4.92 (m, 1H), 3.89 (s, 3H), 3.28-1.81 (m, 4H), 1.33 (s, 9H).

### Example 69

### 4-fluoro-3-methoxy-N-(1-((5-(1-methyl-1H-pyrazol-4-yl) pyridin-3-yl)amino)-3-oxo-2,3-dihydro-1H-inden-4-yl)benzamide

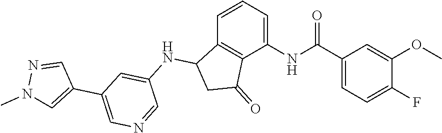

Compound 69 was prepared by the following steps:

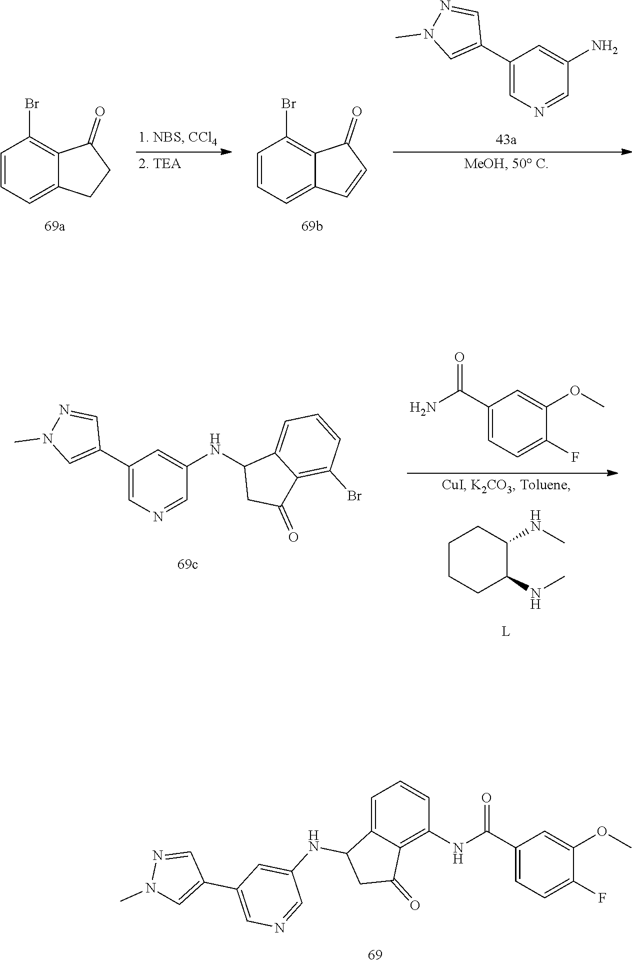

Step 1: To a solution of 69a (2110 mg, 10 mmol) in CCl₄ (25 mL) was added NBS (1958 mg, 11 mmol). The mixture was degassed with N₂ for three times, then refluxed for 4 h. TLC analysis showed the reaction was complete. The reaction mixture was filtered through a pad of Celite, and the filtrate was transferred to the reaction flask protected from light (Use tin foil against the wall of the flask), then Et₃N (5 mL) was added at 0° C. The mixture was allowed to warm to room temperature and stirred for 1 h. TLC analysis showed the reaction was complete. The mixture was concentrated and purified by column chromatography to give product 69b (1643 mg, 79%).

Step 2: To a solution of 43a (1204 mg, 7 mmol) in MeOH (30 mL) was added 69b (731.5 mg, 3.5 mmol). The resulting mixture was heated to 50° C. and stirred for 48 h. TLC analysis showed the reaction was complete, the reaction mixture was concentrated and purified by column chromatography to give product 69c (1685 mg, 63%). ESI-MS (M/S): 383.2 [M+H]⁺.

Step 3: To a solution of 69c (191 mg, 0.5 mmol) in toluene (2 mL) was added 4-Fluoro-3-methoxybenzamide (328 mg, 1.5 mmol), CuI (9.6 mg, 0.05 mmol), (1S, 2S)-(+)-N,N′-Dimethylcyclohexanediamine (14.2 mg, 0.1 mmol) and K₂CO₃ (212 mg, 1.5 mmol). The resulting mixture was degassed with N₂ for three times, then heated to reflux and reacted for 3 h. TLC analysis showed the reaction was complete. The reaction mixture was washed with 5% Ammonium hydroxide twice. The collected organic layer was dried and concentrated, and the residue was purified by column chromatography to give product 69 (121 mg, yield: 51%). ESI-MS (M/S): 472.2 [M+H]⁺. ¹H NMR (62 MHz, acetone-d₆) δ 11.26 (s, 1H), 8.48 (d, J=7.9 Hz, 1H), 8.19-7.26 (m, 10H), 6.44 (d, J=8.4 Hz, 1H), 5.37 (s, 1H), 3.92 (d, J=6.1 Hz, 6H), 2.63 (d, J=8.5 Hz, 1H).

### Example 70

### N-(1-((5-(1-methyl-1H-pyrazol-4-yl) pyridin-3-yl)amino)-3-oxo-2,3-dihydro-1H-inden-4-yl)-4-(trifluoromethyl)benzamide

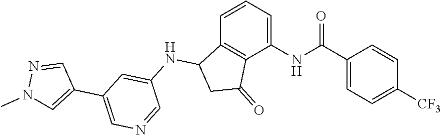 [SMILES: Cn1cc(-c2cncc(NC3CC(=O)c4c(NC(=O)c5ccc(C(F)(F)F)cc5)cccc43)c2)cn1]

The preparation of compound 70 refers to the synthesis of compound 69 in example 69.

ESI-MS (M/S): 492.2 [M+H]⁺.

### Example 71

### N-(3-hydroxy-1-((5-(1-methyl-1H-pyrazol-4-yl) pyridin-3-yl)amino)-2,3-dihydro-1H-inden-4-yl)-4-(trifluoromethyl)benzamide

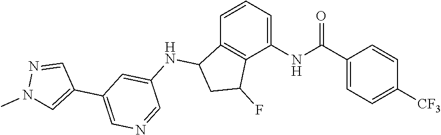 [SMILES: Cn1cc(-c2cncc(NC3CC(F)c4c(NC(=O)c5ccc(C(F)(F)F)cc5)cccc43)c2)cn1]

### N-(3-fluoro-1-((5-(1-methyl-1H-pyrazol-4-yl) pyridin-3-yl)amino)-2,3-dihydro-1H-inden-4-yl)-4-(trifluoromethyl)benzamide

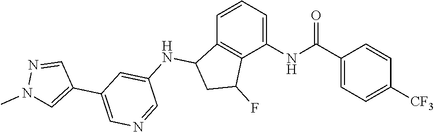 [SMILES: Cn1cc(-c2cncc(NC3CC(F)c4c(NC(=O)c5ccc(C(F)(F)F)cc5)cccc43)c2)cn1]

Compound 71a and compound 71 were prepared by the following steps:

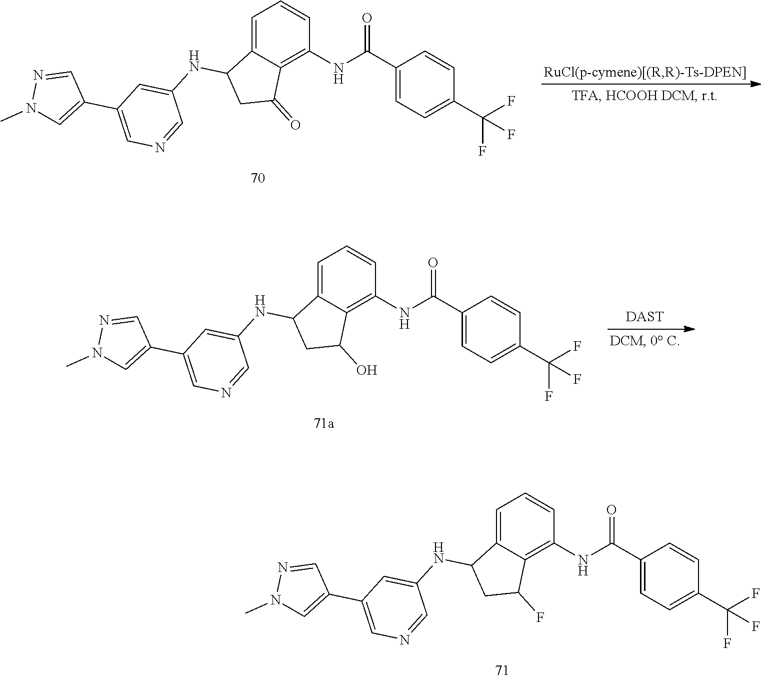

Step 1: To a stirred solution of compound 70 (24.5 mg, 0.05 mmol), Et₃N (0.3 mL) and RuCl(p-cymene) [(R,R)-Ts-DPEN] (3 mg, 0.005 mmol) in DCM (0.5 mL) was added formic acid (0.1 mL) at 0° C. The resulting mixture was stirred at room temperature for overnight. The reaction was complete detected by TLC. After concentration, the reaction mixture was purified on an ISCO chromatography to afford compound 71a as a mixture of stereoisomers (19.9 mg, yield: 79.0%). ESI-MS (M/S): 494.2 [M+H]⁺. ¹H NMR (400 MHz, DMSO-d₆) δ 10.38 (s, 0.5H), 10.23 (s, 0.5H), 8.19-8.05 (m, 4H), 7.98-7.96 (m, 4H), 7.38-7.07 (m, 3H), 6.32 (d, J=8.8 Hz, 0.5H), 6.18 (d, J=8.3 Hz, 1H), 5.76-5.70 (m, 0.5H), 5.52-5.32 (m, 0.5H), 5.35-5.32 (m, 0.5H), 5.29-5.19 (m, 0.5H), 4.96-4.94 (m, 0.5H), 3.87 (d, J=2.6 Hz, 3H), 3.07-3.00 (m, 0.5H), 2.42-2.34 (m, 0.5H), 2.27-2.18 (m, 0.5H), 1.82-1.73 (m, 0.5H).

Step 2: To a solution of compound 71a (15.1 mg, 0.03 mmol) in DCM (0.5 mL) was added DAST (24.2 mg, 0.15 mmol) at 0° C. under N₂. The resulting mixture was continued to stir at that temperature for 2 h. The reaction was complete detected by TLC. After concentration, the resulting mixture was purified on an ISCO chromatography to afford compound 71 as a mixture of stereoisomers (12.5 mg, yield: 84%). ESI-MS (M/S): 496.2 [M+H]⁺. ¹H NMR (400 MHz, DMSO-d₆) δ 10.49-10.46 (m, 1H), 8.20-8.15 (m, 3H), 8.07 (s, 1H), 7.96-7.87 (m, 4H), 7.60-7.45 (m, 2H), 7.31-7.25 (m, 2H), 6.60-6.20 (m, 2H), 3.87 (s, 3H), 3.20-2.71 (m, 1H), 2.27-2.07 (m, 1H), 2.02-1.86 (m, 1H).

### Example 72

### (S)—N-(1-((2-methyl-5-(1-methyl-1H-pyrazol-4-yl) pyridin-3-yl)oxy)-2,3-dihydro-1H-inden-4-yl)-4-(trifluoromethyl)benzamide

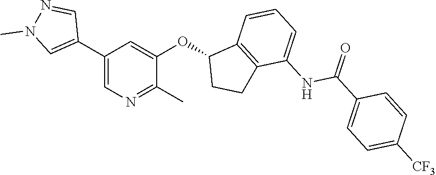 [SMILES: Cc1ncc(-c2cnn(C)c2)cc1O[C@H]1CCc2c(NC(=O)c3ccc(C(F)(F)F)cc3)cccc21]

The preparation of compound 72 refers to the synthesis of compound 15 in example 15.

ESI-MS (M/S): 493.2 [M+H]⁺. ¹H NMR (400 MHz, DMSO-d₆) δ 10.31 (s, 1H), 8.31-8.29 (m, 2H), 8.20-8.18 (m, 2H), 8.01 (s, 1H), 7.94-7.92 (m, 2H), 7.77-7.68 (m, 1H), 7.54-7.43 (m, 1H), 7.39-7.26 (m, 2H), 6.10-6.00 (m, 1H), 3.89 (s, 3H), 3.12-3.05 (m, 1H), 2.98-2.86 (m, 1H), 2.73-2.65 (m, 1H), 2.28 (s, 3H), 2.09-2.01 (m, 1H).

### Example 73

### (S)—N-(3-cyclopropylphenyl)-1-((1-methyl-1H-pyrazolo[4,3-b]pyridin-6-yl)amino)-2,3-dihydro-1H-indene-4-carboxamide

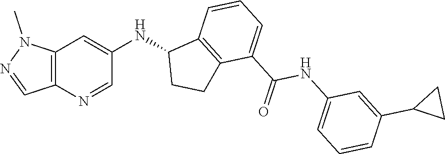 [SMILES: Cn1ncc2ncc(N[C@H]3CCc4c(C(=O)Nc5cccc(C6CC6)c5)cccc43)cc21]

Compound 73 was prepared by the following steps:

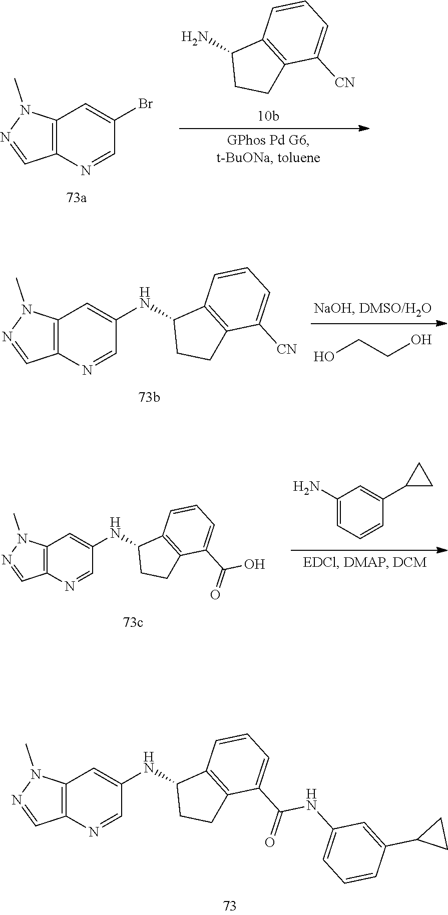

Step 1: To a 100 mL two-necked round bottom flask was added compound 73a (2000 mg, 9.43 mmol), compound 10b (1788 mg, 11.30 mmol), toluene (19 mL), sodium t-butoxide (1267 mg, 13.22 mmol) and GPhos Pd G6 (888 mg, 0.94 mmol) in turn. The resulting mixture was stirred well and degassed with N₂ for three times, then heated to 90° C. and stirred for 3 h. The reaction was complete detected by TLC. After natural cooling to 30° C., the reaction mixture was partitioned between EtOAc (100 mL) and H₂O (100 mL). The aqueous layer was discarded and the separated organic layer was dried over anhydrous sodium sulfate, and concentrated to dryness. The residue was purified on an ISCO chromatography to afford compound 73b (2.3 g, yield: 84.6%). ESI-MS (M/S): 290.1 [M+H]⁺.

Step 2: To a 100 mL flask was added compound 73b (2.3 g, 7.96 mmol), DMSO (12 mL), ethylene glycol (12 mL), H₂O (12 mL) and NaOH (6.0 g, 150.00 mmol) sequentially. The resulting mixture was heated to 110° C. and stirred for 3 h. The reaction was complete detected by TLC. After natural cooling to about 30° C., the pH was adjusted to 6-7 with aqueous HCl (3 M). A large amount of solid was precipitated, and the suspension was stirred for a while, then filtered under reduced pressure. The filter cake was washed with H₂O (6 mL×2) and dried in vacuo at about 50° C. to give compound 73c (2.3 g, yield: 93.1%). ESI-MS (M/S): 307.1 [M−H].

Step 3: To a 4 mL vial was added compound 73c (61 mg, 0.2 mmol), 3-Cyclopropylaniline (53 mg, 0.4 mmol), DMAP (3 mg, 0.02 mmol), CH₂Cl₂ (0.5 mL) and EDCI (77 mg, 0.4 mmol) sequentially. The resulting mixture was stirred at 25° C. for 3 h. The reaction was complete detected by TLC. The reaction mixture was concentrated and the residue was purified on an ISCO chromatography to afford compound 73 (46 mg, yield: 55.3%). ESI-MS (M/S): 424.2 [M+H]⁺. ¹H NMR (62 MHz, Acetone-d₆) δ 9.28 (s, 1H), 8.19 (d, J=2.4 Hz, 1H), 7.94-7.02 (m, 8H), 6.98-6.65 (m, 1H), 6.06-5.68 (m, 1H), 5.46-4.89 (m, 1H), 3.96 (s, 3H), 3.54-3.01 (m, 2H), 2.76-2.21 (m, 1H), 1.93-1.60 (m, 1H), 1.40-1.06 (m, 1H), 1.04-0.47 (m, 4H).

### Example 74

### (S)—N-(4-cyclopropylthiazol-2-yl)-1-((2-methyl-2H-pyrazolo[4,3-b]pyridin-6-yl)amino)-2,3-dihydro-1H-indene-4-carboxamide

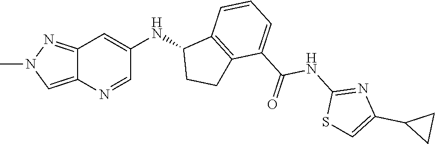 [SMILES: Cn1cc2ncc(N[C@H]3CCc4c(C(=O)Nc5nc(C6CC6)cs5)cccc43)cc2n1]

Compound 74h was prepared by the following steps:

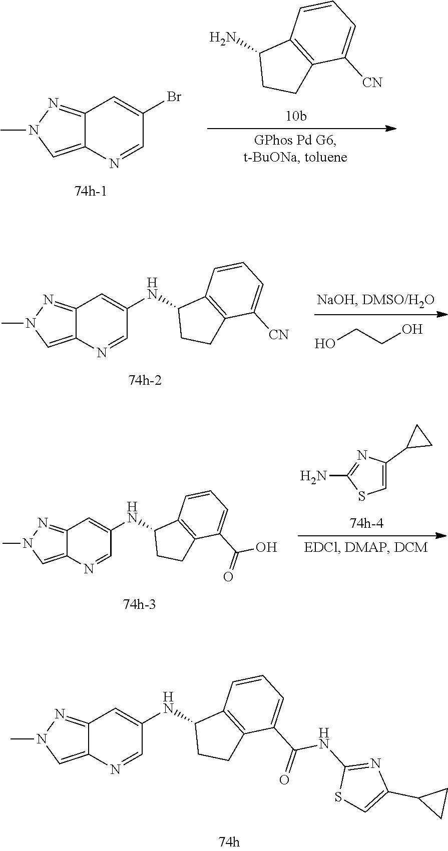

Step 1: To a 100 mL two-necked round bottom flask was added compound 74h-1 (2000 mg, 9.43 mmol), compound 10b (1788 mg, 11.30 mmol), toluene (19 mL), sodium t-butoxide (1267 mg, 13.22 mmol) and GPhos Pd G6 (888 mg, 0.94 mmol) in turn. The resulting mixture was stirred well and degassed with N₂ for three times, then heated to 90° C. and stirred for 3 h. The reaction was complete detected by TLC. After natural cooling to 30° C., the reaction mixture was partitioned between EtOAc (100 mL) and H₂O (100 mL). The aqueous layer was discarded and the separated organic layer was dried over anhydrous sodium sulfate, and concentrated to dryness under reduced pressure. The residue was purified on an ISCO chromatography to afford compound 74h-2 (2.5 g, yield: 92%). ESI-MS (M/S): 290.1 [M+H].

Step 2: To a 100 mL flask was added compound 74h-2 (2.5 g, 8.65 mmol), DMSO (12 mL), ethylene glycol (12 mL), H₂O (12 mL) and NaOH (6.0 g, 150.00 mmol) sequentially. The resulting mixture was heated to 110° C. and stirred for 3 h. The reaction was complete detected by TLC. After natural cooling to about 30° C., the pH was adjusted to 6-7 with aqueous HCl (3 M). A large amount of solid was precipitated, and the suspension was stirred for a while, then filtered under reduced pressure. The filter cake was washed with H₂O (6 mL×2) and dried in vacuo at about 50° C. to give compound 74h-3 (2.4 g, yield: 89.9%). ESI-MS (M/S): 307.1 [M−H]⁻.

Step 3: To a 4 mL vial was added compound 74h-3 (61 mg, 0.2 mmol), compound 74h-4 (56 mg, 0.4 mmol), DMAP (3 mg, 0.02 mmol). CH₂Cl₂ (0.5 mL) and EDCI (77 mg, 0.4 mmol) sequentially. The resulting mixture was stirred at 25° C. for 3 h. The reaction was complete detected by TLC. The reaction mixture was concentrated to dryness under reduced pressure and the residue was purified on an ISCO chromatography to afford compound 74h (57.7 mg, yield: 67.0%).

The preparation of compounds 74a-74i refers to the synthesis of compound 73 in example 73 or compound 74h in example 74.

TABLE 19

| Cpds | Structure | MS [M + 1]⁺ | ¹H NMR |
|---|---|---|---|
| 74a | 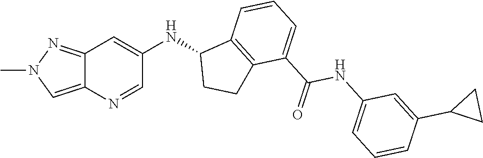 [SMILES: Cn1cc2ncc(N[C@H]3CCc4c(C(=O)Nc5cccc(C6CC6)c5)cccc43)cc2n1] | 424.2 | ¹H NMR (400 MHz, DMSO-d₆) δ 10.13 (s, 1H), 8.32 (s, 1H), 8.18 (d, J = 2.4 Hz, 1H), 7.61 (d, J = 7.6 Hz, 1H), 7.55 (d, J = 8.1 Hz, 1H), 7.52- 7.47 (m, 2H), 7.35 (t, J = 7.5 Hz, 1H), 7.21 (t, J = 7.8 Hz, 1H), 6.90 (d, J = 2.4 Hz, 1H), 6.83 (d, J = 7.6 Hz, 1H), 6.42 (d, J = 8.0 Hz, 1H), 5.12-4.99 (m, 1H), 4.05 (s, 3H), 3.28-3.17 (m, 1H), 3.15-3.00 (m, |
| | | | 1H), 2.63-2.53 (m, 1H), 1.97- |
| | | | 1.80 (m, 2H), 1.02-0.91 (m, 2H), |
| | | | 0.70-0.60 (m, 2H). |
| 74b | 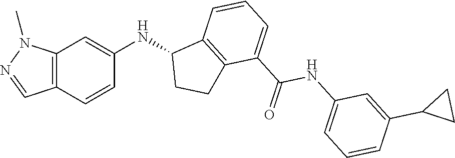 [SMILES: Cn1ncc2ccc(N[C@H]3CCc4c(C(=O)Nc5cccc(C6CC6)c5)cccc43)cc21] | 423.2 | ¹H NMR (62 MHz, Acetone-d₆) δ 9.28 (s, 1H), 7.83-7.04 (m, 8H), 6.99-6.58 (m, 3H), 5.56-4.94 (m, 2H), 3.92 (s, 3H), 3.52-3.01 (m, 2H), 2.74-2.25 (m, 1H), 1.92- 1.55 (m, 1H), 1.40-1.11 (m, 1H), 1.09-0.55 (m, 4H). |
| 74c | 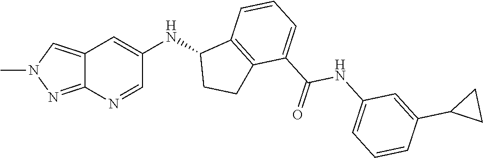 [SMILES: Cn1cc2cc(N[C@H]3CCc4c(C(=O)Nc5cccc(C6CC6)c5)cccc43)cnc2n1] | 424.2 | ¹H NMR (62 MHz, Acetone-d₆) δ 9.31 (s, 1H), 8.32 (d, 1H), 7.90 (s, 1H), 7.80-6.66 (m, 8H), 5.49- 4.83 (m, 2H), 4.13 (s, 3H), 3.51- 3.02 (m, 2H), 2.72-2.29 (m, 1H), 1.92-1.59 (m, 1H), 1.45-1.11 (m, 1H), 1.11-0.48 (m, 4H). |
| 74d | 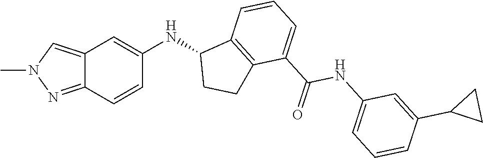 [SMILES: Cn1cc2cc(N[C@H]3CCc4c(C(=O)Nc5cccc(C6CC6)c5)cccc43)ccc2n1] | 423.2 | ¹H NMR (62 MHz, Acetone-d₆) δ 9.26 (s, 1H), 7.94-6.63 (m, 11H), 5.28-4.74 (m, 2H), 4.10 (s, 3H), 3.48-3.00 (m, 2H), 2.72-2.31 (m, 1H), 1.77 (d, J = 5.0 Hz, 1H), 1.44- 1.17 (m, 1H), 1.09-0.47 (m, 4H). |
| 74e | 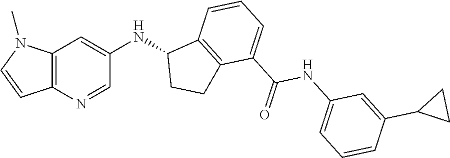 [SMILES: Cn1ccc2ncc(N[C@H]3CCc4c(C(=O)Nc5cccc(C6CC6)c5)cccc43)cc21] | 423.2 | ¹H NMR (400 MHz, DMSO-d₆) δ 10.12 (s, 1H), 8.01 (d, J = 2.3 Hz, 1H), 7.64-7.45 (m, 4H), 7.33 (t, J = 7.6 Hz, 1H), 7.27-7.18 (m, 2H), 7.15 (d, J = 2.3 Hz, 1H), 6.83 (d, J = 7.6 Hz, 1H), 6.35 (d, J = 3.2 Hz, 1H), 5.95 (d, J = 8.7 Hz, 1H), 5.11 (q, J = 7.7 Hz, 1H), 3.71 (s, 3H), 3.23 (m, J = 16.3, 8.4, 3.3 Hz, 1H), 3.13-2.99 (m, 1H), 2.61 (m, J = 3.9 |
| | | | Hz, 1H), 1.95-1.77 (m, 2H), 1.00- |
| | | | 0.92 (m, 2H), 0.69-0.62 (m, 2H). |
| 74f | 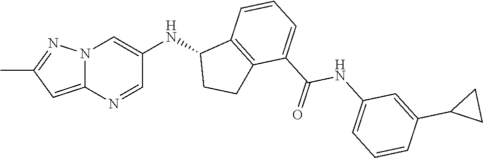 [SMILES: Cc1cc2ncc(N[C@H]3CCc4c(C(=O)Nc5cccc(C6CC6)c5)cccc43)cn2n1] | 424.2 | ¹H NMR (62 MHz, Acetone-d₆) δ 9.29 (s, 1H), 8.30 (d, J = 2.7 Hz, 1H), 8.13 (d, J = 2.7 Hz, 1H), 7.79- 7.04 (m, 7H), 6.85 (dt, J = 7.5, 1.6 Hz, 1H), 6.27 (s, 1H), 5.52-4.77 (m, 2H), 3.51-3.06 (m, 2H), 2.74- 2.43 (m, 1H), 2.37 (s, 3H), 1.98- 1.64 (m, 1H), 1.17-0.48 (m, 4H). |
| 74g | 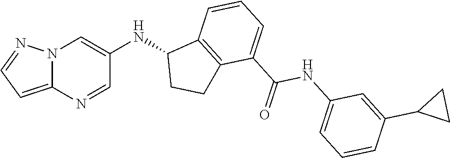 [SMILES: O=C(Nc1cccc(C2CC2)c1)c1cccc2c1CC[C@@H]2Nc1cnc2ccnn2c1] | 410.2 | ¹H NMR (62 MHz, Acetone-d₆) δ 9.30 (s, 1H), 8.38 (d, J = 2.6 Hz, 1H), 8.23 (d, J = 2.7 Hz, 1H), 7.85 (d, J = 2.4 Hz, 1H), 7.79-7.39 (m, 5H), 7.38-7.05 (m, 2H), 6.98- 6.72 (m, 1H), 6.57-6.43 (m, 1H), 5.68-4.86 (m, 2H), 3.54-3.04 (m, 2H), 2.76-2.40 (m, 1H), 1.96- 1.51 (m, 2H), 1.22-0.51 (m, 4H). |
| 74h | 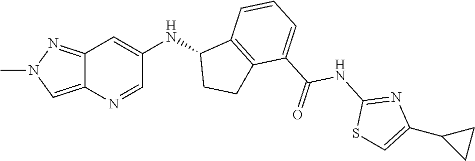 [SMILES: Cn1cc2ncc(N[C@H]3CCc4c(C(=O)Nc5nc(C6CC6)cs5)cccc43)cc2n1] | 431.2 | ¹H NMR (62 MHz, Acetone-d₆) δ 12.42 (s, 1H), 8.31 (s, 1H), 8.16 (d, J = 2.4 Hz, 1H), 7.82-7.70 (m, 1H), 7.67-7.13 (m, 2H), 6.89-6.83 (m, 2H), 6.40 (d, J = 8.2 Hz, 1H), 5.11- 4.99 (m, 1H), 4.05 (s, 3H), 3.25- 3.05 (m, 2H), 2.12-1.91 (m, 2H), 0.92-0.78 (m, 4H). |
| 74i | 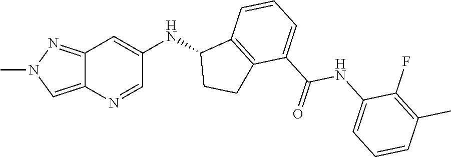 | 416.2 | ¹H NMR (400 MHz, DMSO-d₆) δ 9.94 (s, 1H), 8.32 (s, 1H), 8.24- 8.12 (m, 1H), 7.67 (d, J = 7.6 Hz, 1H), 7.52-7.48 (m, 2H), 7.35 (t, J = 7.6 Hz, 1H), 7.16-7.08 (m, 2H), 6.90 (s, 1H), 6.42 (d, J = 8.0 Hz, 1H), 5.08-5.03 (m, 1H), 4.05 (s, 3H), 3.30-3.22 (m, 1H), 3.15- 3.06 (m, 1H), 2.63-2.55 (m, 1H), 2.28 (s, 3H), 1.91-1.82 (m, 1H). |

### Example 75

### (S)—N-(2-fluoro-3-methylphenyl)-1-((1-methyl-1H-pyrazolo[4,3-b]pyridin-6-yl)oxy)-2,3-dihydro-1H-indene-4-carboxamide

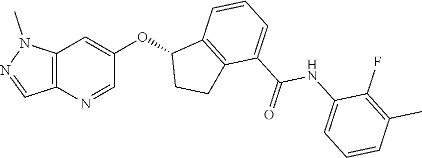 [SMILES: Cc1cccc(NC(=O)c2cccc3c2CC[C@@H]3Oc2cnc3cnn(C)c3c2)c1F]

Compound 75 was prepared by the following steps:

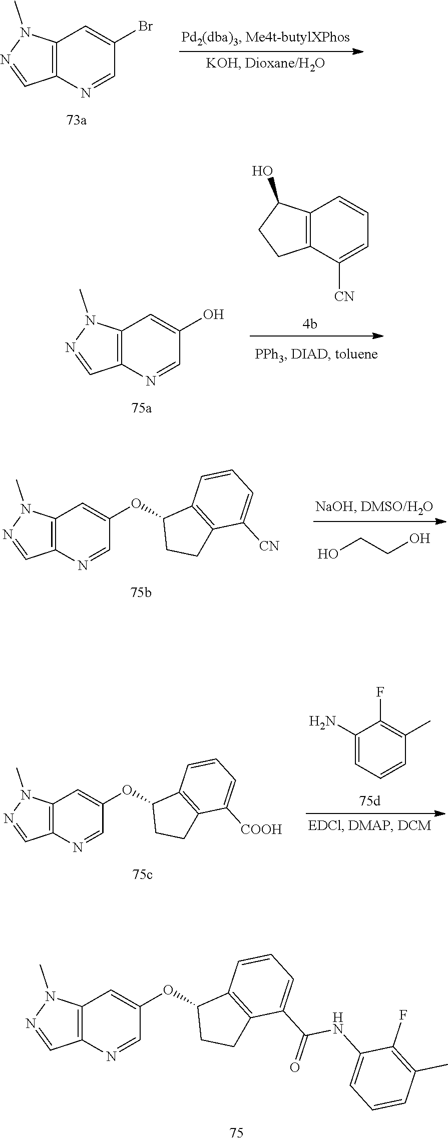

Step 1: To a 250 mL three-necked flask was added compound 73a (2360 mg, 10.0 mmol), 1,4-dioxane (27 mL), H₂O (9 mL), KOH (1680 mg, 30.0 mmol), Meat-butylXPhos (674 mg, 1.4 mmol), and Pd₂(dba)₃ (229 mg, 0.25 mmol) sequentially. The mixture was stirred well and degassed with N₂ for three times, then heated to 90° C. and stirred for 3 h. The reaction was complete detected by TLC. After natural cooling to about 30° C., the separated upper organic phase was discarded, and the aqueous phase was diluted with water (9 mL) and washed with CH₂Cl₂ (20 mL), then adjusted to pH=8-9 with concentrated HCl, further adjusted to 6˜7 with 3 N aqueous HCl. A large amount of solid was precipitated and the suspension was stirred for a while, then filtered under reduced pressure. The filter cake was washed with water (9 mL) and dried in vacuo at about 50° C. to give compound 75a (1495 mg, yield: 99.5%). ESI-MS (M/S): 150.1 [M+H].

Step 2: To a 100 mL two-necked flask was added compound 75a (1490 mg, 10.0 mmol), compound 4b (1749 mg, 11.0 mmol), PPh₃ (3935 mg, 15.0 mmol), and toluene (20 mL) sequentially. The mixture was stirred well and degassed with N₂ for three times, then cooled to −15˜−20° C. DIAD (3034 mg, 15.0 mmol) was then added dropwise to the above mixture and the reaction temperature was kept below −10° C. during the dropwise addition. The resulting mixture was stirred at −15-−10° C. for 3 h. The reaction was complete detected by TLC. The reaction mixture was concentrated to dryness to obtain thick liquid which was redissolved in EtOH (6 mL). After being stirred for a moment, a large amount of solid was precipitated. The resulting suspension was continued to stir at 25° C. for 2 h, then filtered. The filter cake was washed with EtOH (3 mL) and dried in vacuo at about 50° C. to give compound 75b (1970 mg, yield: 67.9%). ESI-MS (M/S): 291.1 [M+H]⁺.

Step 3: To a 100 mL flask was sequentially added compound 75b (1.67 g, 5.7 mmol), DMSO (8.5 mL), ethylene glycol (8.5 mL), H₂O (8.5 mL), and NaOH (4.0 g, 100.0 mmol) in turn. The resulting mixture was heated to 110° C. and stirred at 110° C. for about 3 h. The reaction was complete detected by TLC. After natural cooling to 30° C., the pH was adjusted to 6-7 with 3 N aqueous HCl. A large amount of solid was precipitated. The suspension was stirred for a moment, then filtered under reduced pressure. The filter cake was washed with H₂O (4.5 mL×2) and dried in vacuo at about 50° C. to give compound 75c (1.67 g, yield: 95.1%). ESI-MS (M/S): 308.1 [M−H]⁻.

Step 4: To a 4 mL vial was added compound 75c (62 mg, 0.2 mmol), compound 75d (38 mg, 0.3 mmol), DMAP (3 mg, 0.02 mmol), CH₂Cl₂ (0.5 mL) and EDCI (77 mg, 0.4 mmol) sequentially. The resulting mixture was stirred at 25° C. for 3 h. The reaction was complete detected by TLC. The reaction mixture was concentrated to dryness under reduced pressure and the residue was purified on an ISCO chromatography to afford compound 75 (25 mg, yield: 30.8%). ESI-MS (M/S): 417.2 [M+H]⁺. ¹H NMR (400 MHz, DMSO-d₆) δ 10.01 (s, 1H), 8.24 (s, 1H), 8.16 (s, 1H), 7.90 (s, 1H), 7.77 (d, J=7.6 Hz, 1H), 7.67 (d, J=7.5 Hz, 1H), 7.51 (t, J=7.2 Hz, 1H), 7.43 (t, J=7.6 Hz, 1H), 7.18-7.06 (m, 2H), 6.12-6.03 (m, 1H), 4.07 (s, 3H), 3.33-3.27 (m, 1H), 3.25-3.11 (m, 1H), 2.76-2.66 (m, 1H), 2.28 (s, 3H), 2.20-2.09 (m, 1H).

### Example 76

### (S)—N-(5-chloropyridin-3-yl)-1-((1-methyl-1H-pyrazolo[4,3-b]pyridin-6-yl)oxy)-2,3-dihydro-1H-indene-4-carboxamide

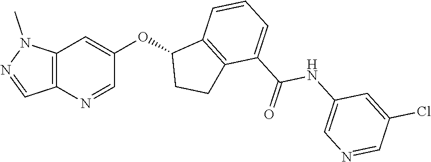 [SMILES: Cn1ncc2ncc(O[C@H]3CCc4c(C(=O)Nc5cncc(Cl)c5)cccc43)cc21]

Compound 76ac was prepared by the following steps:

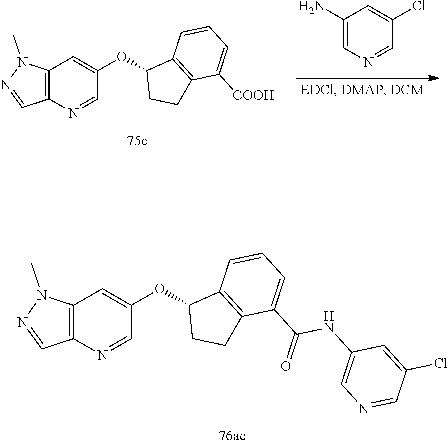 [SMILES: Cn1ncc2ncc(O[C@H]3CCc4c(C(=O)O)cccc43)cc21 ; Nc1cncc(Cl)c1 ; Cn1ncc2ncc(O[C@H]3CCc4c(C(=O)Nc5cncc(Cl)c5)cccc43)cc21]

To a 4 mL vial was added compound 75c (31 mg, 0.1 mmol), 3-amino-5-chloropyridine (25 mg, 0.2 mmol), DMAP (3 mg, 0.02 mmol), CH₂Cl₂ (0.5 mL) and EDCI (38 mg, 0.2 mmol) sequentially. The resulting mixture was stirred at 25° C. for 3 h. The reaction was complete detected by TLC. The reaction mixture was concentrated to dryness under reduced pressure and the residue was purified on an ISCO chromatography to afford compound 76ac (26 mg, yield: 61.0%).

The preparation of compounds 76aa-76be refer to the synthesis of compound 75 in example 75 or compound 76ac in example 76.

TABLE 20

| | | MS | |
|---|---|---|---|
| Cpds | Structure | [M + 1]⁺ | ¹H NMR |
| 76aa | 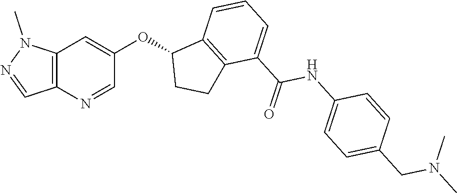 [SMILES: CN(C)Cc1ccc(NC(=O)c2cccc3c2CC[C@@H]3Oc2cnc3cnn(C)c3c2)cc1] | 442.2 | ¹H NMR (400 MHz, DMSO-d₆) δ 10.27 (s, 1H), 8.24 (s, 1H), 8.16 (s, 1H), 7.90 (s, 1H), 7.71 (d, J = 7.9 Hz, 3H), 7.66 (d, J = 7.5 Hz, 1H), 7.43 (t, J = 7.6 Hz, 1H), 7.26 (d, J = 8.2 Hz, 2H), 6.08-6.06 (m, 1H), 4.07 (s, 3H), 3.30-3.27 (m, 1H), 3.16- 3.13 (m, 1H), 2.76-2.64 (m, 1H), 2.16- 2.14 (m, 7H). |
| 76ab | 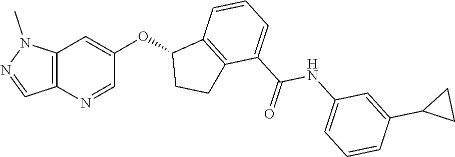 [SMILES: Cn1ncc2ncc(O[C@H]3CCc4c(C(=O)Nc5cccc(C6CC6)c5)cccc43)cc21] | 425.2 | ¹H NMR (400 MHz, DMSO-d₆) δ 10.20 (s, 1H), 8.23 (s, 1H), 8.15 (s, 1H), 7.89 (s, 1H), 7.71-7.64 (m, 2H), 7.59-7.37 (m, 3H), 7.21 (s, 1H), 6.83 (s, 1H), 6.07 (s, 1H), 4.07 (s, 3H), 3.29-3.16 (m, 2H), 2.73-2.68 (m, 1H), 2.15-2.12 (m, 1H), 1.93-1.88 (m, 1H), 1.02 (s, 2H), 0.65 (s, 2H). |
| 76ac | 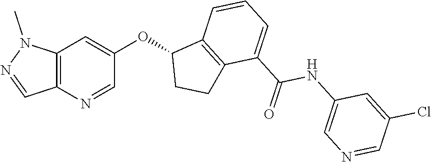 [SMILES: Cn1ncc2ncc(O[C@H]3CCc4c(C(=O)Nc5cncc(Cl)c5)cccc43)cc21] | 420.1 | ¹H NMR (400 MHz, DMSO-d₆) δ 10.71 (s, 1H), 8.85 (s, 1H), 8.44-8.37 (m, 2H), 8.24 (s, 1H), 8.16 (s, 1H), 7.90 (s, 1H), 7.79 (d, J = 7.7 Hz, 1H), 7.71 (d, J = 7.5 Hz, 1H), 7.47 (t, J = 7.6 Hz, 1H), 6.11- 6.05 (m, 1H), 4.07 (s, 3H), 3.41-3.35 (m, 1H), 3.25-3.12 (m, 1H), 2.76-2.64 (m, 1H), 2.24-2.10 (m, 1H). |
| 76ad | 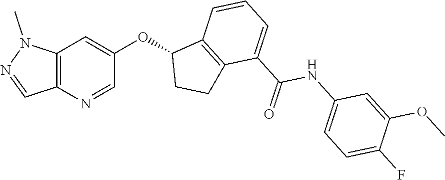 [SMILES: COc1cc(NC(=O)c2cccc3c2CC[C@@H]3Oc2cnc3cnn(C)c3c2)ccc1F] | 433.2 | ¹H NMR (400 MHz, DMSO-d₆) δ 10.32 (s, 1H), 8.24 (s, 1H), 8.16 (s, 1H), 7.90 (s, 1H), 7.77-7.65 (m, 3H), 7.44 (t, J = 7.6 Hz, 1H), 7.37-7.28 (m, 1H), 7.24-7.13 (m, 1H), 6.13-6.02 (m, 1H), 4.07 (s, 3H), 3.84 (s, 3H), 3.32-3.27 (m, 1H), 3.22- 3.09 (m, 1H), 2.79-2.63 (m, 1H), 2.21- 2.08 (m, 1H). |
| 76ae | 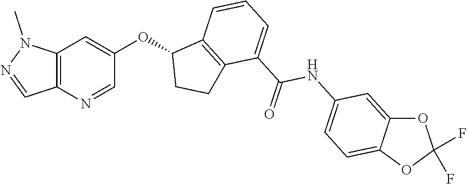 [SMILES: Cn1ncc2ncc(O[C@H]3CCc4c(C(=O)Nc5ccc6c(c5)OC(F)(F)O6)cccc43)cc21] | 465.1 | ¹H NMR (400 MHz, DMSO-d₆) δ 10.51 (s, 1H), 8.24 (s, 1H), 8.16 (s, 1H), 7.95- 7.87 (m, 2H), 7.73 (d, J = 7.6 Hz, 1H), 7.68 (d, J = 7.5 Hz, 1H), 7.51-7.39 (m, 3H), 6.11-6.04 (m, 1H), 4.07 (s, 3H), 3.33-3.26 (m, 1H), 3.22-3.09 (m, 1H), 2.77-2.63 (m, 1H), 2.20-2.10 (m, 1H). |
| 76af | 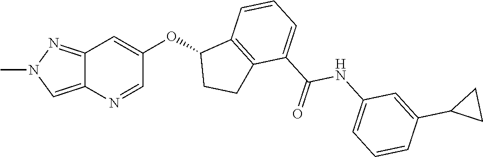 [SMILES: Cn1cc2ncc(O[C@H]3CCc4c(C(=O)Nc5cccc(C6CC6)c5)cccc43)cc2n1] | 425.2 | ¹H NMR (400 MHz, DMSO-d₆) δ 10.19 (s, 1H), 8.55 (s, 1H), 8.22 (s, 1H), 7.70 (d, J = 7.5 Hz, 1H), 7.68-7.61 (m, 1H), 7.56 (d, J = 8.1 Hz, 1H), 7.51 (s, 1H), 7.41 (t, J = 7.5 Hz, 1H), 7.21 (t, J = 7.8 Hz, 1H), 6.82 (d, J = 7.6 Hz, 1H), 6.09-5.96 (m, 1H), 4.16 (s, 1H), 3.37-3.27 (m, 1H), 3.19-3.10 (m, 1H), 2.68-2.60 (m, 1H), 2.17-2.09 (m, 1H), 1.92-1.87 (m, 1H), 0.99-0.93 (m, 2H), 0.67-0.63 (m, 2H). |
| 76ag | 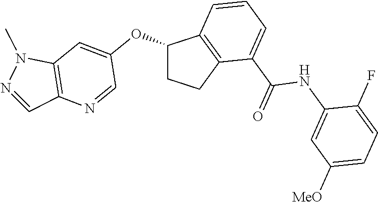 [SMILES: COc1ccc(F)c(NC(=O)c2cccc3c2CC[C@@H]3Oc2cnc3cnn(C)c3c2)c1] | 433.1 | ¹H NMR (400 MHz, DMSO-d₆) δ 10.05 (s, 1H), 8.24 (s, 1H), 8.15 (s, 1H), 7.90 (s, 1H), 7.77 (d, J = 7.5 Hz, 1H), 7.67 (d, J = 7.6 Hz, 1H), 7.43 (t, J = 7.6 Hz, 1H), 7.34- 7.31 (m, 1H), 7.22 (t, J = 9.6 Hz, 1H), 6.86-6.76 (m, 1H), 6.09-6.05 (m, 1H), 4.07 (s, 3H), 3.76 (s, 3H), 3.24-3.13 (m, 1H), 2.75-2.68 (m, 1H), 2.18-2.12 (m, 1H). |
| 76ah | 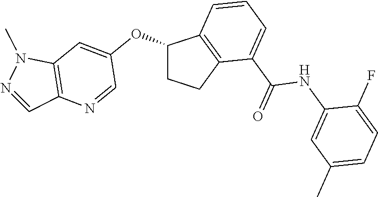 [SMILES: Cc1ccc(F)c(NC(=O)c2cccc3c2CC[C@@H]3Oc2cnc3cnn(C)c3c2)c1] | 417.2 | ¹H NMR (400 MHz, DMSO-d₆) δ 10.02 (s, 1H), 8.25-8.12 (m, 1H), 8.15 (s, 1H), 7.90 (s, 1H), 7.76 (d, J = 7.5 Hz, 1H), 7.67 (d, J = 7.5 Hz, 1H), 7.50 (s, 1H), 7.43 (t, J = 7.5 Hz, 1H), 7.16 (t, J = 7.5 Hz, 1H), 7.08-7.03 (m, 1H), 6.09-6.04 (m, 1H), 4.07 (s, 3H), 3.35-3.30 (m, 1H), 3.19- 3.16 (m, 1H), 2.74-2.68 (m, 1H), 2.31 (s, 3H), 2.18-2.11 (m, 1H). |
| 76ai | 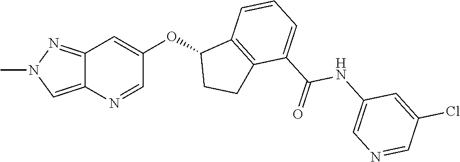 [SMILES: Cn1cc2ncc(O[C@H]3CCc4c(C(=O)Nc5cncc(Cl)c5)cccc43)cc2n1] | 420.1 | ¹H NMR (400 MHz, DMSO-d₆) δ 10.71 (s, 1H), 8.85 (s, 1H), 8.55 (s, 1H), 8.41- 8.38 (m, 2H), 8.21 (s, 1H), 7.78 (d, J = 7.6 Hz, 1H), 7.72-7.62 (m, 2H), 7.46 (t, J = 7.6 Hz, 1H), 6.04-6.01 (m, 1H), 4.16 (s, 3H), 3.34-3.27 (m, 1H), 3.20-3.13 (m, 1H), 2.69-2.60 (m, 1H), 2.17-2.10 (m, 1H). |
| 76aj | 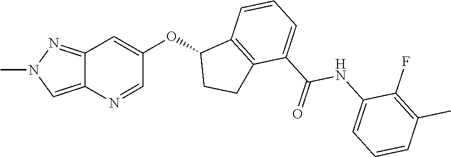 | 417.2 | ¹H NMR (400 MHz, DMSO-d₆) δ 10.01 (s, 1H), 8.55 (s, 1H), 8.20 (s, 1H), 7.75 (d, J = 7.4 Hz, 1H), 7.65 (d, J = 7.4 Hz, 2H), 7.50 (t, J = 7.6 Hz, 1H), 7.42 (t, J = 7.6 Hz, 1H), 7.16-7.08 (m, 2H), 6.03-6.01 (m, 1H), 4.15 (s, 3H), 3.35-3.28 (m, 1H), 3.21-3.12 (m, 1H), 2.67-2.62 (m, 1H), 2.27 (s, 3H), 2.14-2.10 (m, 1H). |
| 76ak | 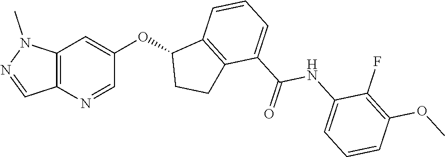 [SMILES: COc1cccc(NC(=O)c2cccc3c2CC[C@@H]3Oc2cnc3cnn(C)c3c2)c1F] | 433.2 | ¹H NMR (400 MHz, DMSO-d₆) δ 10.06 (s, 1H), 8.24 (s, 1H), 8.15 (s, 1H), 7.90 (s, 1H), 7.77 (d, J = 7.6 Hz, 1H), 7.67 (d, J = 7.5 Hz, 1H), 7.43 (t, J = 7.6 Hz, 1H), 7.23 (t, J = 7.2 Hz, 1H), 7.14 (t, J = 8.2 Hz, 1H), 7.04 (t, J = 7.9 Hz, 1H), 6.10-6.02 (m, 1H), 4.07 (s, 3H), 3.87 (s, 3H), 3.34-3.27 (m, 1H), 3.22-3.11 (m, 1H), 2.74-2.65 (m, 1H), 2.19-2.09 (m, 1H). |
| 76al | 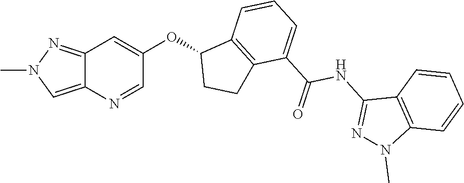 [SMILES: Cn1cc2ncc(O[C@H]3CCc4c(C(=O)Nc5nn(C)c6ccccc56)cccc43)cc2n1] | 439.2 | ¹H NMR (400 MHz, DMSO-d₆) δ 10.72 (s, 1H), 8.55 (s, 1H), 8.22 (s, 1H), 7.84 (d, J = 7.6 Hz, 1H), 7.78 (d, J = 8.2 Hz, 1H), 7.70-7.60 (m, 3H), 7.47-7.37 (m, 2H), 7.13 (t, J = 7.5 Hz, 1H), 6.11-5.98 (m, 1H), 4.16 (s, 3H), 4.01 (s, 3H), 3.33-3.27 (m, 1H), 3.27-3.17 (m, 1H), 2.73-2.61 (m, 1H), 2.20-2.08 (m, 1H). |
| 76am | 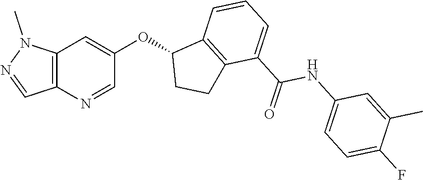 [SMILES: Cc1cc(NC(=O)c2cccc3c2CC[C@@H]3Oc2cnc3cnn(C)c3c2)ccc1F] | 417.2 | ¹H NMR (400 MHz, DMSO-d₆) δ 10.26 (s, 1H), 8.24 (s, 1H), 8.16 (s, 1H), 7.89 (s, 1H), 7.76-7.68 (m, 2H), 7.66 (d, J = 7.5 Hz, 1H), 7.61-7.51 (m, 1H), 7.43 (t, J = 7.6 Hz, 1H), 7.13 (t, J = 9.2 Hz, 1H), 6.14- 6.01 (m, 1H), 4.07 (s, 3H), 3.34-3.26 (m, 1H), 3.21-3.07 (m, 1H), 2.77-2.65 (m, 1H), 2.25 (s, 3H), 2.21-2.06 (m, 1H). |
| 76an | 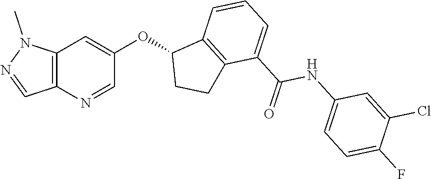 [SMILES: Cn1ncc2ncc(O[C@H]3CCc4c(C(=O)Nc5ccc(F)c(Cl)c5)cccc43)cc21] | 437.1 | ¹H NMR (400 MHz, DMSO-d6) δ 10.50 (s, 1H), 8.24 (s, 1H), 8.16 (s, 1H), 8.13- 8.05 (m, 1H), 7.89 (s, 1H), 7.78-7.64 (m, 3H), 7.51-7.38 (m, 2H), 6.11-6.02 (m, 1H), 4.07 (s, 3H), 3.34-3.27 (m, 1H), 3.22-3.09 (m, 1H), 2.77-2.64 (m, 1H), 2.22-2.07 (m, 1H). |
| 76ao | 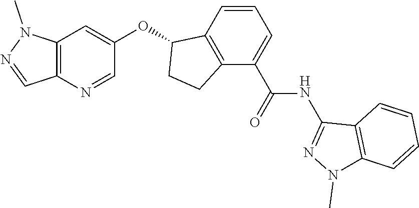 [SMILES: Cn1ncc2ncc(O[C@H]3CCc4c(C(=O)Nc5nn(C)c6ccccc56)cccc43)cc21] | 439.2 | ¹H NMR (400 MHz, DMSO-d6) δ 10.74 (s, 1H), 8.25 (s, 1H), 8.16 (s, 1H), 7.91 (s, 1H), 7.86 (d, J = 7.6 Hz, 1H), 7.79 (d, J = 8.2 Hz, 1H), 7.68 (d, J = 7.5 Hz, 1H), 7.62 (d, J = 8.5 Hz, 1H), 7.49-7.38 (m, 2H), 7.13 (t, J = 7.5 Hz, 1H), 6.13-6.04 (m, 1H), 4.08 (s, 3H), 4.02 (s, 3H), 3.45-3.34 (m, 1H), 3.30-3.18 (m, 1H), 2.79-2.65 (m, 1H), 2.22-2.10 (m, 1H). |
| 76ap | 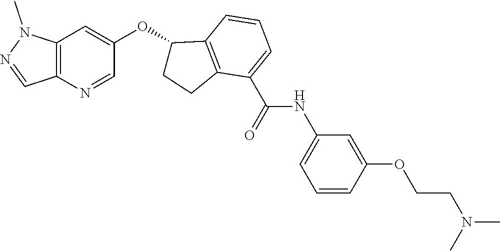 [SMILES: CN(C)CCOc1cccc(NC(=O)c2cccc3c2CC[C@@H]3Oc2cnc3cnn(C)c3c2)c1] | 472.2 | ¹H NMR (400 MHz, DMSO-d6) δ 10.25 (s, 1H), 8.24 (s, 1H), 8.16 (s, 1H), 7.90 (s, 1H), 7.76-7.64 (m, 2H), 7.50-7.40 (m, 2H), 7.33 (d, J = 8.2 Hz, 1H), 7.24 (t, J = 8.1 Hz, 1H), 6.73-6.64 (m, 1H), 6.14- 6.00 (m, 1H), 4.07 (s, 3H), 4.04 (t, J = 5.7 Hz, 2H), 3.33-3.27 (m, 1H), 3.23-3.09 (m, 1H), 2.77-2.67 (m, 1H), 2.64 (t, J = 5.8 Hz, 2H), 2.23 (s, 6H), 2.21-2.08 (m, 1H). |
| 76aq | 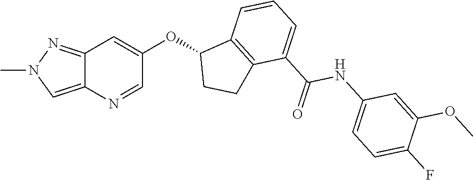 | 433.2 | ¹H NMR (400 MHz, DMSO-d₆) δ 10.31 (s, 1H), 8.55 (s, 1H), 8.21 (s, 1H), 7.77- 7.60 (m, 4H), 7.43 (t, J = 7.6 Hz, 1H), 7.34- 7.31 (m, 1H), 7.21-7.16 (m, 1H), 6.03- 6.01 (m, 1H), 4.16 (s, 3H), 3.84 (s, 3H), 3.34-3.28 (m, 1H), 3.18-3.11 (m, 1H), 2.66-2.62 (m, 1H), 2.14-2.09 (m, 1H). |
| 76ar | 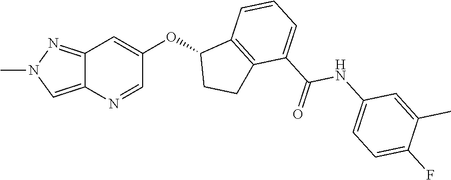 | 417.2 | ¹H NMR (400 MHz, DMSO-d₆) δ 10.26 (s, 1H), 8.55 (s, 1H), 8.21 (s, 1H), 7.75- 7.50 (m, 5H), 7.42 (t, J = 7.6 Hz, 1H), 7.12 (t, J = 9.2 Hz, 1H), 6.03-6.01 (m, 1H), 4.16 (s, 3H), 3.36 (s, 3H), 3.34-3.25 (m, 1H), 3.18-3.10 (m, 1H), 2.66-2.61 (m, 1H), 2.16-2.10 (m, 1H). |
| 76as | 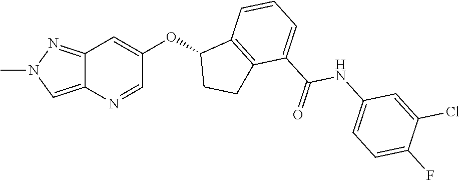 [SMILES: Cn1cc2ncc(O[C@H]3CCc4c(C(=O)Nc5ccc(F)c(Cl)c5)cccc43)cc2n1] | 437.1 | ¹H NMR (400 MHz, DMSO-d₆) δ 10.49 (s, 1H), 8.55 (s, 1H), 8.21 (s, 1H), 8.10- 8.07 (m, 1H), 7.76-7.57 (m, 4H), 7.46- 7.40 (m, 2H), 6.03-6.01 (m, 1H), 4.16 (s, 3H), 3.32-3.25 (m, 1H), 3.16-3.12 (m, 1H), 2.66-2.61 (m, 1H), 2.16-2.12 (m, 1H). |
| 76at | 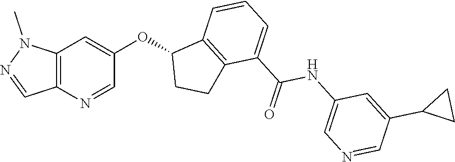 [SMILES: Cn1ncc2ncc(O[C@H]3CCc4c(C(=O)Nc5cncc(C6CC6)c5)cccc43)cc21] | 426.2 | ¹H NMR (400 MHz, DMSO-d6) δ 10.40 (s, 1H), 8.70 (s, 1H), 8.24 (s, 1H), 8.19- 8.13 (m, 2H), 7.89 (s, 1H), 7.84 (s, 1H), 7.76 (d, J = 7.6 Hz, 1H), 7.68 (d, J = 7.5 Hz, 1H), 7.45 (t, J = 7.6 Hz, 1H), 6.13- 6.03 (m, 1H), 4.07 (s, 3H), 3.34-3.28 (m, 1H), 3.23-3.09 (m, 1H), 2.77-2.63 (m, 1H), 2.21-2.08 (m, 1H), 2.04-1.92 (m, 1H), 1.07-0.97 (m, 2H), 0.79-0.65 (m, 2H). |
| 76au | 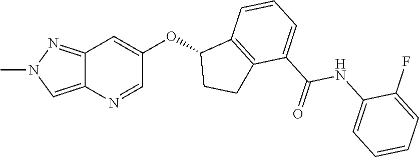 [SMILES: Cn1cc2ncc(O[C@H]3CCc4c(C(=O)Nc5ccccc5F)cccc43)cc2n1] | 403.2 | ¹H NMR (400 MHz, DMSO-d₆) δ 10.08 (s, 1H), 8.55 (s, 1H), 8.21 (s, 1H), 7.77 (d, J = 7.5 Hz, 1H), 7.74-7.61 (m, 4H), 7.43 (t, J = 7.5 Hz, 1H), 7.33-7.20 (m, 3H), 6.03-6.01 (m, 1H), 3.31-3.29 (m, 1H), 3.20-3.16 (m, 1H), 2.67-2.63 (m, 1H), 2.15-2.11 (m, 1H). |
| 76av | 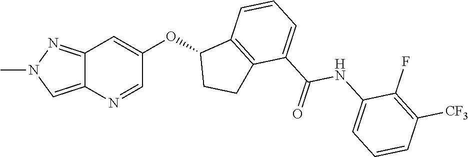 [SMILES: Cn1cc2ncc(O[C@H]3CCc4c(C(=O)Nc5cccc(C(F)(F)F)c5F)cccc43)cc2n1] | 471.1 | ¹H NMR (400 MHz, DMSO-d₆) δ 10.38 (s, 1H), 8.55 (s, 1H), 8.21 (s, 1H), 8.00 (s, 1H), 7.80 (s, 1H), 7.69-7.63 (m, 3H), 7.48-7.40 (m, 2H), 6.06-6.04 (m, 1H), 4.16 (s, 3H), 3.19-3.16 (m, 1H), 2.68- 2.64 (m, 1H), 2.44-2.32 (m, 1H), 2.14- 2.10 (m, 1H). |
| 76aw | 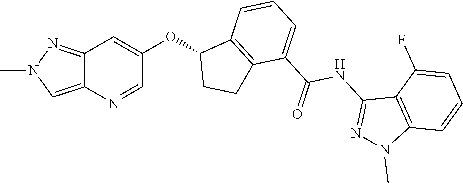 [SMILES: Cn1cc2ncc(O[C@H]3CCc4c(C(=O)Nc5nn(C)c6cccc(F)c56)cccc43)cc2n1] | 457.2 | ¹H NMR (400 MHz, DMSO-d₆) δ 10.54 (s, 1H), 8.55 (s, 1H), 8.22 (s, 1H), 7.83- 7.78 (m, 1H), 7.68-7.67 (m, 2H), 7.51- 7.39 (m, 3H), 6.92-6.84 (m, 1H), 6.03- 6.02 (m, 1H), 4.16 (s, 3H), 4.04 (s, 3H), 3.32 (s, 1H), 3.21-3.18 (m, 1H), 2.68- 2.66 (m, 1H), 2.14-2.11 (m, 1H). |
| 76ax | 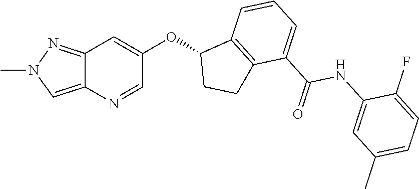 [SMILES: Cc1ccc(F)c(NC(=O)c2cccc3c2CC[C@@H]3Oc2cnc3cn(C)nc3c2)c1] | 417.2 | 1HNMR (400 MHz, DMSO-d₆) δ 10.00 (s, 1H), 8.55 (s, 1H), 8.21 (s, 1H), 7.75 (d, J = 7.6 Hz, 1H), 7.66-7.64 (m, 2H), 7.51- 7.49 (m, 1H), 7.42-7.39 (m, 1H), 7.21- 7.13 (m, 1H), 7.07 (s, 1H), 6.07-5.99 (m, 1H), 4.16 (s, 3H), 3.30-3.28 (m, 1H), 3.17-3.09 (m, 1H), 2.67-2.62 (m, 1H), 2.31 (s, 3H), 2.16-2.08 (m, 1H). |
| 76ay | 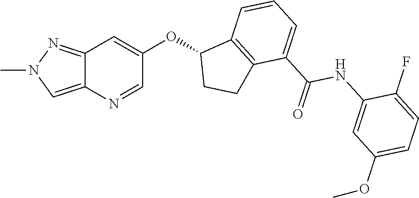 [SMILES: COc1ccc(F)c(NC(=O)c2cccc3c2CC[C@@H]3Oc2cnc3cn(C)nc3c2)c1] | 433.2 | ¹H NMR (400 MHz, DMSO-d₆) δ 9.90 (s, 1H), 8.55 (s, 1H), 8.21 (s, 1H), 7.75 (d, J = 7.6 Hz, 1H), 7.65 (s, 2H), 7.50-7.39 (m, 2H), 6.96-6.92 (m, 1H), 6.82-6.80 (m, 1H), 6.03-6.01 (m, 1H), 4.16 (s, 3H), 3.79 (s, 3H), 3.18-3.14 (m, 2H), 2.66- 2.62 (m, 1H), 2.20-2.09 (m, 1H). |
| 76az | 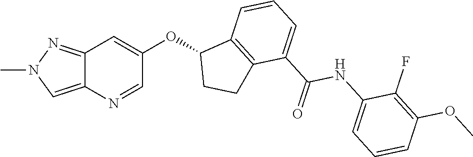 | 433.2 | ¹H NMR (400 MHz, DMSO-d₆) δ 10.06 (s, 1H), 8.55 (s, 1H), 8.21 (s, 1H), 7.76 (d, J = 7.7 Hz, 1H), 7.65 (d, J = 7.7 Hz, 2H), 7.42 (t, J = 7.6 Hz, 1H), 7.23 (t, J = 7.3 Hz, 1H), 7.13 (t, J = 8.3 Hz, 1H), 7.04 (t, J = 7.9 Hz, 1H), 6.03-6.01 (m, 1H), 4.16 (s, 3H), 3.87 (s, 3H), 3.31-3.27 (m, 1H), 3.19-3.15 (m, 1H), 2.67-2.62 (m, 1H), 2.15-2.11 (m, 1H). |
| 76ba | 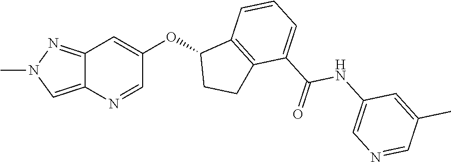 | 400.2 | ¹H NMR (400 MHz, DMSO-d₆) δ 10.45 (s, 1H), 8.70 (s, 1H), 8.55 (s, 1H), 8.21 (s, 1H), 8.17 (s, 1H), 8.07 (s, 1H), 7.75 (d, J = 7.6 Hz, 1H), 7.70-7.60 (m, 2H), 7.44 (t, J = 7.6 Hz, 1H), 6.02-6.01 (m, 1H), 4.16 (s, 3H), 3.29-2.26 (m, 1H), 3.21- 3.08 (m, 1H), 2.66-2.62 (m, 1H), 2.32 (s, 3H), 2.15-2.11 (m, 1H). |
| 76bb | 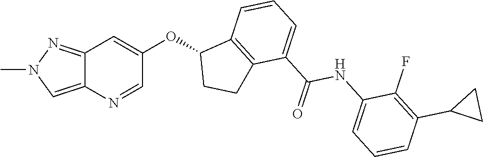 [SMILES: Cn1cc2ncc(O[C@H]3CCc4c(C(=O)Nc5cccc(C6CC6)c5F)cccc43)cc2n1] | 443.2 | ¹H NMR (400 MHz, DMSO-d₆) δ 10.01 (s, 1H), 8.56 (s, 1H), 8.24 (s, 1H), 7.77 (d, J = 7.6 Hz, 1H), 7.70 (s, 1H), 7.65 (d, J = 7.5 Hz, 1H), 7.49-7.41 (m, 2H), 7.09 (t, J = 7.9 Hz, 1H), 6.84 (t, J = 7.4 Hz, 1H), 6.03-6.02 (m, 1H), 3.35-3.31 (m, 1H), 3.19-3.14 (m, 1H), 2.66-2.62 (m, 1H), 2.14-2.09 (m, 2H), 1.02-0.97 (m, 2H), 0.76-0.72 (m, 2H). |
| 76bc | 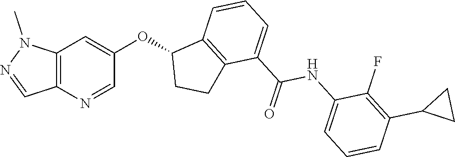 [SMILES: Cn1ncc2ncc(O[C@H]3CCc4c(C(=O)Nc5cccc(C6CC6)c5F)cccc43)cc21] | 443.2 | ¹H NMR (400 MHz, DMSO-d6) δ 10.01 (s, 1H), 8.24 (s, 1H), 8.16 (s, 1H), 7.90 (s, 1H), 7.77 (d, J = 7.7 Hz, 1H), 7.68 (d, J = 7.5 Hz, 1H), 7.51-7.39 (m, 2H), 7.10 (t, J = 7.9 Hz, 1H), 6.84 (t, J = 7.3 Hz, 1H), 6.13-6.03 (m, 1H), 4.07 (s, 3H), 3.43- 3.37 (m, 1H), 3.25-3.13 (m, 1H), 2.78- 2.65 (m, 1H), 2.21-2.04 (m, 2H), 1.06- 0.96 (m, 2H), 0.80-0.73 (m, 2H). |
| 76bd | 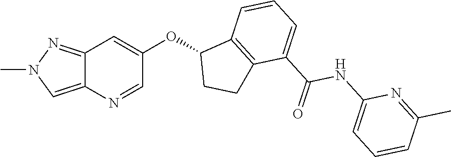 | 400.2 | ¹H NMR (400 MHz, DMSO-d6) δ 10.69 (s, 1H), 8.24 (s, 1H), 8.15 (s, 1H), 8.02 (d, J = 8.2 Hz, 1H), 7.89 (s, 1H), 7.81-7.70 (m, 2H), 7.64 (d, J = 7.6 Hz, 1H), 7.39 (t, J = 7.6 Hz, 1H), 7.03 (d, J = 7.5 Hz, 1H), 6.10-6.00 (m, 1H), 4.07 (s, 3H), 3.32- 3.28 (m, 1H), 3.23-3.11 (m, 1H), 2.75- 2.63 (m, 1H), 2.45 (s, 3H), 2.21-2.08 (m, 1H). |
| 76be | 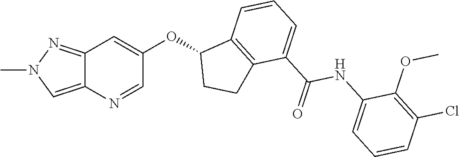 [SMILES: COc1c(Cl)cccc1NC(=O)c1cccc2c1CC[C@@H]2Oc1cnc2cn(C)nc2c1] | 449.1 | ¹H NMR (400 MHz, DMSO-d₆) δ 9.79 (s, 1H), 8.55 (s, 1H), 8.22 (s, 1H), 7.87-7.81 (m, 1H), 7.77 (d, J = 7.6 Hz, 1H), 7.67- 7.65 (m, 2H), 7.44 (t, J = 7.5 Hz, 1H), 7.34 (d, J = 8.0 Hz, 1H), 7.19 (t, J = 8.1 Hz, 1H), 6.07-5.99 (m, 1H), 4.16 (s, 3H), 3.83 (s, 3H), 3.36-3.31 (m, 1H), 3.24- 3.16 (m, 1H), 2.71-2.62 (m, 1H), 2.18- 2.21 (m, 1H). |

### Example 77

### (S)-3-cyclopropyl-N-(1-((1-methyl-1H-pyrazolo[4,3-b]pyridin-6-yl)oxy)-2,3-dihydro-1H-inden-4-yl)benzamide

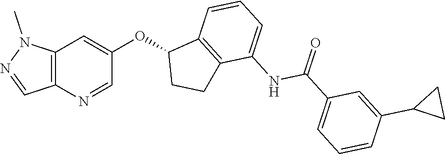 [SMILES: Cn1ncc2ncc(O[C@H]3CCc4c(NC(=O)c5cccc(C6CC6)c5)cccc43)cc21]

Compound 77 was prepared by the following steps:

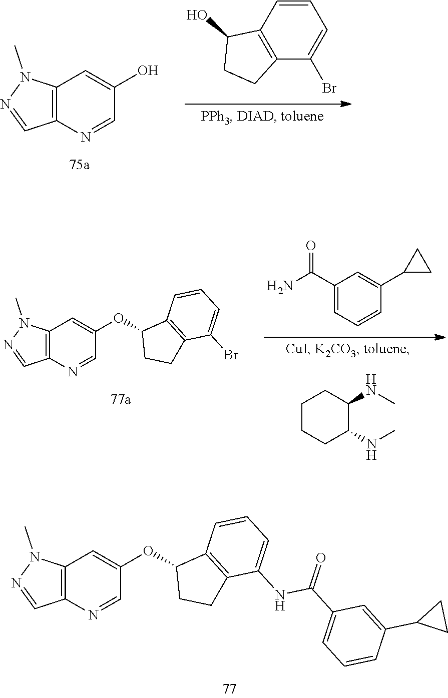

Step 1: To a 100 mL two-necked flask was sequentially added compound 75a (1490 mg, 10.0 mmol), (R)-4-bromo-2,3-dihydro-1H-inden-1-ol (2556 mg, 12.0 mmol), PPh₃ (3935 mg, 15.0 mmol) and toluene (20 mL). The mixture was degassed with N₂ for three times sufficient stirring, then cooled to −15-−20° C. DIAD (3034 mg, 15.0 mmol) was added dropwise into the above mixture, and the reaction temperature was kept below −10° C. during the dropwise addition. After the dropwise addition, the mixture was stirred at −15˜−10° C. for about 3 hours. The reaction was complete detected by TLC. The reaction mixture was concentrated to obtain thick liquid, which was redissolved in EtOH (20 mL) and stirred for a moment. A large amount of solid was precipitated, and the resulting suspension was continued to stir for 2 h at 25° C. After filtering, the filter cake was washed with EtOH (3 mL) and dried in vacuo at about 50° C. to give compound 77a (2476 mg, yield: 72.0%). ESI-MS (M/S): 344.1 [M+H]⁺.

Step 2: To a 4 mL vial was sequentially added compound 77a (68 mg, 0.2 mmol), 3-cyclopropylbenzamide (65 mg, 0.4 mmol), (1R,2R)—N,N′-Dimethyl-1,2-cyclohexanediamine (6 mg, 0.04 mmol), toluene (0.2 mL), K₂CO₃ (83 mg, 0.6 mmol) and CuI (4 mg, 0.02 mmol) in turn. The mixture was degassed with N₂ for three times with sufficient stirring, then heated to 90° C. and stirred until the reaction was complete detected by TLC. After natural cooling to 30° C., the reaction mixture was diluted with CH₂Cl₂ (10 mL), washed with 13% ammonium hydroxide (10 mL). The aqueous phase was discarded and the organic phase was dried over Na₂SO₄, then concentrated to dryness under reduced pressure. The residue was purified on an ISCO chromatography to obtain compound 77 (38 mg, yield: 38.0%). ESI-MS (M/S): 425.2 [M+H]⁺. ¹H NMR (400 MHz, DMSO-d₆) δ 10.03 (s, 1H), 8.25 (d, J=2.3 Hz, 1H), 8.15 (s, 1H), 7.88 (s, 1H), 7.74 (d, J=7.7 Hz, 1H), 7.67 (s, 1H), 7.48-7.28 (m, 5H), 6.08-6.07 (m, 1H), 4.07 (s, 3H), 3.08-3.04 (m, 1H), 2.93-2.89 (m, 1H), 2.71-2.68 (m, 1H), 2.14-2.10 (m, 1H), 2.05-2.02 (m, 1H), 1.02-1.00 (m, 2H), 0.79-0.77 (m, 2H).

### Example 78

The preparation of compounds 78a and 78b refer to the synthesis of compound 77 in example 77.

TABLE 21

| | | MS | |
|---|---|---|---|
| Cpds | Structure | [M + 1]⁺ | ¹HNMR |
| 78a | 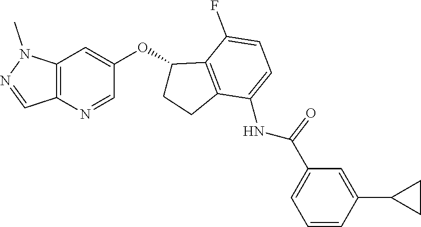 [SMILES: Cn1ncc2ncc(O[C@H]3CCc4c(NC(=O)c5cccc(C6CC6)c5)ccc(F)c43)cc21] | 443.2 | ¹H NMR (400 MHz, DMSO-d₆) δ 10.05 (s, 1H), 8.23 (d, J = 2.3 Hz, 1H), 8.16 (s, 1H), 7.85 (s, 1H), 7.73 (d, J = 7.7 Hz, 1H), 7.66 (s, 1H), 7.52-7.48 (m, 1H), 7.41 (t, J = 7.6 Hz, 1H), 7.32 (d, J = 7.8 Hz, 1H), 7.15 (t, J = 8.7 Hz, 1H), 6.25-6.24 (m, 1H), 4.06 (s, 3H), 3.18- 3.10 (m, 1H), 2.96-2.90 (m, 1H), 2.66- 2.57 (m, 1H), 2.26-2.20 (m, 1H), 2.06- 2.00 (m, 1H), 1.06-0.96 (m, 2H), 0.79-0.74 (m, 2H). |
| 78b | 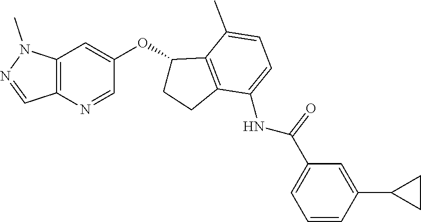 [SMILES: Cc1ccc(NC(=O)c2cccc(C3CC3)c2)c2c1[C@@H](Oc1cnc3cnn(C)c3c1)CC2] | 439.2 | ¹H NMR (400 MHz, DMSO-d₆) δ 9.94 (s, 1H), 8.24 (s, 1H), 8.16 (s, 1H), 7.86- 7.79 (m, 1H), 7.73 (d, J = 7.7 Hz, 1H), 7.66 (s, 1H), 7.44-7.35 (m, 2H), 7.31 (d, J = 7.7 Hz, 1H), 7.14 (d, J = 8.0 Hz, 1H), 6.09-6.08 (m, 1H), 4.07 (s, 3H), 3.12-3.06 (m, 1H), 2.97-2.83 (m, 1H), 2.62-2.56 (m, 2H), 2.30 (s, 3H), 2.06-1.98 (m, 1H), 1.08-0.97 (m, 2H), 0.84-0.73 (m, 2H). |

### Example 79

### (S)—N-(2-fluoro-3-methylphenyl)-1-(methyl (1-methyl-1H-pyrazolo[4,3-b]pyridin-6-yl)amino)-2,3-dihydro-1H-indene-4-carboxamide

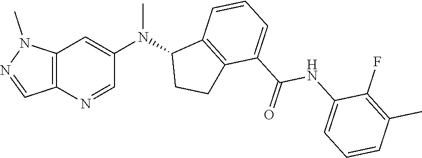 [SMILES: Cc1cccc(NC(=O)c2cccc3c2CC[C@@H]3N(C)c2cnc3cnn(C)c3c2)c1F]

Compound 79 was prepared by the following steps:

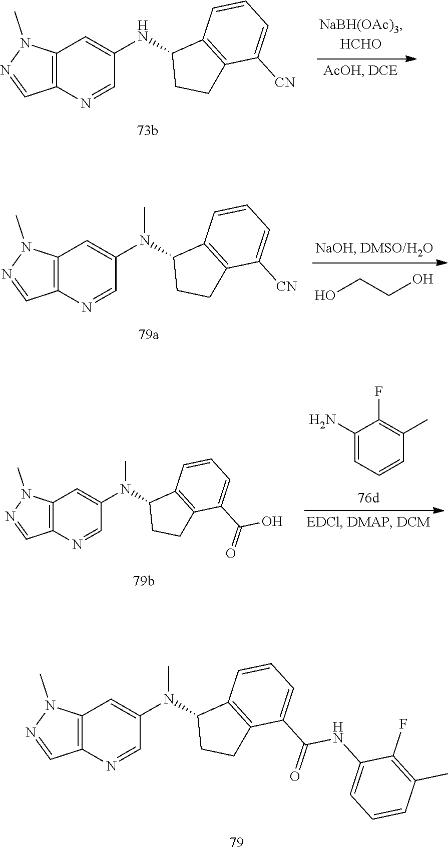

Step 1: To a 250 mL flask was sequentially added compound 73% (2.3 g, 7.96 mmol), 40% formalin (720 mg, 24.0 mmol), 1,2-dichloroethane (80 mL), and AcOH (8 mL), followed by addition of sodium triacetoxy borohydride (5.1 g, 24.0 mmol) in portions with stirring at 25° C. The resulting mixture was stirred at 25° C. for 1 h. TLC showed the starting material was not consumed. 40% formalin (72 mg, 2.4 mmol) and sodium triacetoxyborohydride (510 mg, 2.4 mmol) were added sequentially and the mixture was continued to stir at 25° C. for 1 h. TLC showed the starting material remained unconsumed. 40% formalin (360 mg, 12.0 mmol), sodium triacetoxy borohydride (2.5 g, 12.0 mmol) and 1,2-dichloroethane (20 mL) was added again and the mixture was stirred at 25° C. for another 1 h. The reaction was complete detected by TLC. The reaction mixture was diluted with CH₂Cl₂ (100 mL), sequentially washed with 5% aq. NaOH (100 mL×3) and water (100 mL) and dried over anhydrous Na₂SO₄. After concentration to dryness under reduced pressure, the residue was purified on an ISCO chromatography to afford compound 79a (1.7 g, yield: 70.4%).

Step 2: To a 100 mL flask was sequentially added compound 79a (1.3 g, 4.3 mmol), DMSO (8 mL), ethylene glycol (4 mL), H₂O (4 mL) and NaOH (2.0 g, 50.0 mmol). The resulting mixture was heated to 110° C. and stirred for about 3 h. The reaction was complete detected by TLC. After natural cooling to 30° C., the pH was adjusted to 6-7 with 3 M aqueous HCl. A large amount of solid was precipitated and the suspension was stirred for a moment, then filtered. The filter cake was washed with H₂O (4 mL×2) and dried in vacuo at about 50° C. to give compound 79b (1.1 g, yield: 81.1%). ESI-MS (M/S): 321.1 [M−H]⁻.

Step 3: To a 4 mL vial was sequentially added compound 79b (64 mg, 0.2 mmol), compound 75d (50 mg, 0.4 mmol), DMAP (3 mg, 0.02 mmol), CH₂Cl₂ (0.5 mL) and EDCI (77 mg, 0.4 mmol). The resulting mixture was stirred at 25° C. for 3 h. The reaction was complete detected by TLC. The mixture was concentrated to dryness and the residue was purified on an ISCO chromatography to afford compound 79 (36 mg, yield: 41.8%). ESI-MS (M/S): 430.2 [M+H]⁺. ¹H NMR (62 MHz, DMSO-d₆) δ 9.02 (s, 1H), 8.54 (d, J=2.6 Hz, 1H), 8.24-7.56 (m, 3H), 7.56-6.79 (m, 5H), 6.01-5.62 (m, 1H), 3.98 (s, 3H), 3.76-2.95 (m, 2H), 2.78 (s, 3H), 2.66-2.36 (m, 1H), 2.30 (s, 3H), 1.90-1.11 (m, 1H).

### Example 80

### (S)—N-(5-chloropyridin-3-yl)-1-(methyl (1-methyl-1H-pyrazolo[4,3-b]pyridin-6-yl)amino)-2,3-dihydro-1H-indene-4-carboxamide

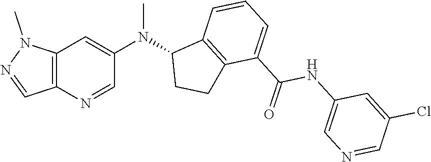 [SMILES: CN(c1cnc2cnn(C)c2c1)[C@H]1CCc2c(C(=O)Nc3cncc(Cl)c3)cccc21]

Compound 80e was prepared by the following steps:

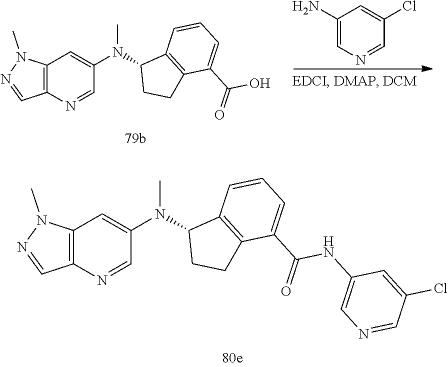 [SMILES: CN(c1cnc2cnn(C)c2c1)[C@H]1CCc2c(C(=O)O)cccc21 ; Nc1cncc(Cl)c1 ; CN(c1cnc2cnn(C)c2c1)[C@H]1CCc2c(C(=O)Nc3cncc(Cl)c3)cccc21]

To a 4 mL vial was sequentially added compound 79b (64 mg, 0.2 mmol), 3-amino-5-chloropyridine (50 mg, 0.4 mmol), DMAP (3 mg, 0.02 mmol), CH₂Cl₂ (0.5 mL) and EDCI (77 mg, 0.4 mmol). The resulting mixture was stirred at 25° C. for 3 h. The reaction was complete detected by TLC. The mixture was concentrated to dryness under reduced pressure and the residue was purified on an ISCO chromatography to afford compound 80e (53 mg, yield: 62.1%).

The preparation of compounds 80a-80y refers to the synthesis of compound 79 in example 79 or compound 80e in example 80.

TABLE 22

| | | MS | |
|---|---|---|---|
| Cpds | Structure | [M + 1]⁺ | ¹HNMR |
| 80a | 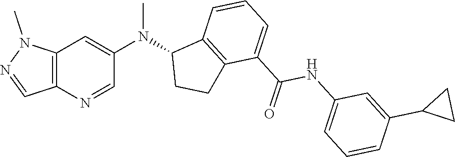 [SMILES: CN(c1cnc2cnn(C)c2c1)[C@H]1CCc2c(C(=O)Nc3cccc(C4CC4)c3)cccc21] | 438.2 | ¹H NMR (400 MHz, DMSO-d₆) δ 10.15 (s, 1H), 8.54 (d, J = 2.5 Hz, 1H), 8.03 (s, 1H), 7.61 (d, J = 7.4 Hz, 1H), 7.57-7.46 (m, 2H), 7.41-7.28 (m, 3H), 7.21 (t, J = 7.9 Hz, 1H), 6.83 (d, J = 7.7 Hz, 1H), 5.82 (t, J = 7.9 Hz, 1H), 3.98 (s, 3H), 3.29- 3.20 (m, 1H), 3.12-3.02 (m, 1H), 2.70 (s, 3H), 2.48-2.40 (m, 1H), 2.06-1.96 (m, 1H), 1.96-1.85 (m, 1H), 1.00-0.91 (m, 2H), 0.68-0.62 (m, 2H). |
| 80b | 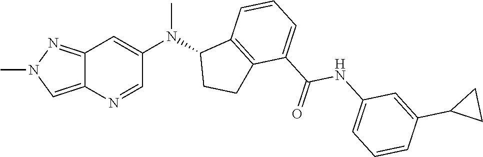 [SMILES: CN(c1cnc2cn(C)nc2c1)[C@H]1CCc2c(C(=O)Nc3cccc(C4CC4)c3)cccc21] | 438.2 | ¹H NMR (62 MHz, DMSO-d₆) δ 10.12 (s, 1H), 8.63 (d, J = 2.5 Hz, 1H), 8.42 (s, 1H), 7.70-7.43 (m, 3H), 7.43-7.24 (m, 3H), 7.25-7.04 (m, 2H), 6.92-6.70 (m, 1H), 5.73-5.56 (m, 1H), 4.10 (s, 3H), 3.21- 2.89 (m, 2H), 2.63 (s, 3H), 2.11-1.76 (m, 2H), 1.06-0.91 (m, 1H), 0.89-0.74 (m, 1H), 0.71-0.55 (m, 2H). |
| 80c | 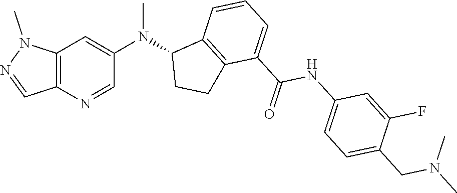 [SMILES: CN(C)Cc1ccc(NC(=O)c2cccc3c2CC[C@@H]3N(C)c2cnc3cnn(C)c3c2)cc1F] | 473.2 | ¹HNMR (400 MHz, DMSO-d₆) δ 10.44 (s, 1H), 8.54(s, 1H), 8.04(s, 1H), 7.72 (d, J = 12.4 Hz, 1H), 7.64 (d, J = 7.3 Hz, 1H), 7.51 (d, J = 8.1 Hz, 1H), 7.40-7.28 (m, 4H), 5.87-5.81 (m, 1H), 3.98 (s, 3H), 3.40 (s, 2H), 3.29-3.22 (m, 1H), 3.13- 3.05 (m, 1H), 2.70 (s, 3H), 2.48-2.44 (m, 1H), 2.15 (s, 6H), 2.03-2.00 (m, 1H). |
| 80d | 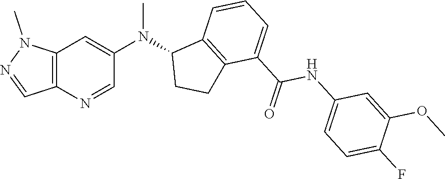 [SMILES: COc1cc(NC(=O)c2cccc3c2CC[C@@H]3N(C)c2cnc3cnn(C)c3c2)ccc1F] | 446.2 | ¹H NMR (62 MHz, Acetone-d₆) δ 9.46 (s, 1H), 8.53 (d, J = 2.6 Hz, 1H), 8.02-6.86 (m, 8H), 5.81 (t, J = 7.8 Hz, 1H), 3.98 (s, 3H), 3.89 (s, 3H), 3.54-2.96 (m, 2H), 2.77 (s, 3H), 2.71-2.16 (m, 2H). |
| 80e | 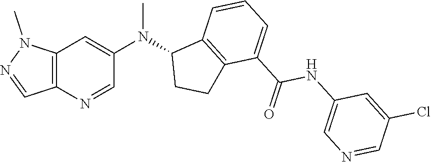 [SMILES: CN(c1cnc2cnn(C)c2c1)[C@H]1CCc2c(C(=O)Nc3cncc(Cl)c3)cccc21] | 433.2 | ¹H NMR (62 MHz, acetone-d₆) δ 9.82 (s, 1H), 8.84 (d, J = 2.2 Hz, 1H), 8.55-8.48 (m, 2H), 8.33 (d, J = 2.3 Hz, 1H), 7.92 (s, 1H), 7.85-7.62 (m, 1H), 7.55-7.19 (m, 3H), 5.96-5.62 (m, 1H), 3.98 (s, 3H), 3.40-3.15 (m, 2H), 2.78 (s, 3H), 2.63- 1.98 (m, 2H) |
| 80f | 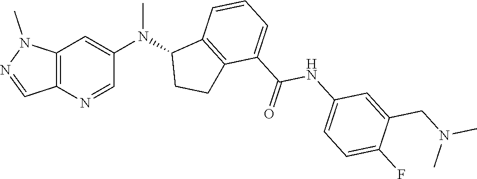 [SMILES: CN(C)Cc1cc(NC(=O)c2cccc3c2CC[C@@H]3N(C)c2cnc3cnn(C)c3c2)ccc1F] | 473.2 | ¹H NMR (400 MHz, DMSO-d₆) δ 10.29 (s, 1H), 8.54 (s, 1H), 8.03 (s, 1H), 7.86- 7.84 (m, 1H), 7.71-7.67 (m, 1H), 7.64 (d, J = 7.4 Hz, 1H), 7.38 (t, J = 7.5 Hz, 1H), 7.35-7.29 (m, 2H), 7.16 (t, J = 9.3 Hz, 1H), 5.84-5.80 (m, 1H), 3.98 (s, 3H), 3.46 (s, 2H), 3.29-3.24 (m, 1H), 3.09- 3.05 (m, 1H), 2.70 (s, 3H), 2.48-2.44 (m, 1H), 2.21 (s, 6H), 2.02-1.98 (m, 1H). |
| 80g | 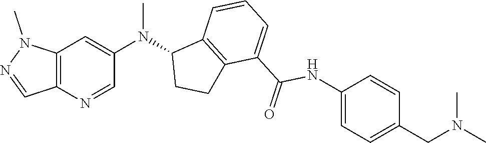 [SMILES: CN(C)Cc1ccc(NC(=O)c2cccc3c2CC[C@@H]3N(C)c2cnc3cnn(C)c3c2)cc1] | 455.3 | ¹H NMR (62 MHz, acetone-d₆) δ 9.43 (s, 1H), 8.53 (d, J = 2.6 Hz, 1H), 8.03-6.35 (m, 9H), 5.97-5.64 (m, 1H), 3.98 (s, 3H), 3.38 (s, 2H), 3.28-2.89 (m, 2H), 2.77 (s, 3H), 2.66-2.27 (m, 1H), 2.19 (s, 6H), 1.95-1.78 (m, 1H). |
| 80h | 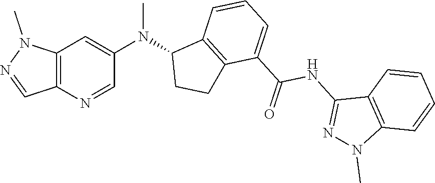 [SMILES: CN(c1cnc2cnn(C)c2c1)[C@H]1CCc2c(C(=O)Nc3nn(C)c4ccccc34)cccc21] | 452.2 | ¹H NMR (62 MHz, acetone-d₆) δ 9.79 (s, 1H), 8.55 (d, J = 2.5 Hz, 1H), 8.21-6.92 (m, 9H), 6.03-5.64 (m, 1H), 4.18-3.84 (m, 6H), 3.71-3.05 (m, 2H), 2.79 (s, 3H), 2.68-1.80 (m, 2H). |
| 80i | 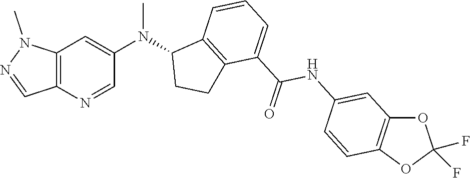 [SMILES: CN(c1cnc2cnn(C)c2c1)[C@H]1CCc2c(C(=O)Nc3ccc4c(c3)OC(F)(F)O4)cccc21] | 478.2 | ¹H NMR (62 MHz, acetone-d₆) δ 9.66 (s, 1H), 8.67-8.41 (m, 1H), 8.18-7.02 (m, 8H), 6.11-5.53 (m, 1H), 3.98 (s, 3H), 3.62-2.97 (m, 2H), 2.77 (s, 3H), 2.66- 1.79 (m, 1H). |
| 80j | 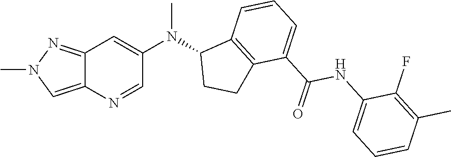 | 430.2 | ¹H NMR (400 MHz, DMSO-d₆) δ 9.96 (s, 1H), 8.64 (s, 1H), 8.43 (s, 1H), 7.67 (d, J = 7.2 Hz, 1H), 7.50 (t, J = 6.9 Hz, 1H), 7.40-7.30 (m, 2H), 7.17-7.07 (m, 3H), 5.75-5.66 (m, 1H), 4.11 (s, 3H), 3.32- 3.22 (m, 1H), 3.15-3.02 (m, 1H), 2.63 (s, 3H), 2.44-2.33 (m, 1H), 2.28 (s, 3H), 2.06-1.94 (m, 1H). |
| 80k | 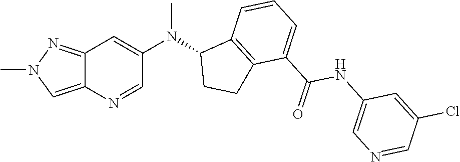 [SMILES: CN(c1cnc2cn(C)nc2c1)[C@H]1CCc2c(C(=O)Nc3cncc(Cl)c3)cccc21] | 433.2 | ¹H NMR (400 MHz, DMSO-d₆) δ 10.68 (s, 1H), 8.84 (s, 1H), 8.64 (s, 1H), 8.43 (s, 1H), 8.40 (s, 1H), 8.40-8.36 (m, 1H), 7.69 (d, J = 7.2 Hz, 1H), 7.43-7.36 (m, 2H), 7.14 (s, 1H), 5.75-5.72 m, 1H), 4.11 (s, 3H), 3.30-3.18 (m, 1H), 3.12-3.08 (m, 1H), 2.63 (s, 3H), 2.44-2.37 (m, 1H), 2.04-2.00 (m, 1H). |
| 80l | 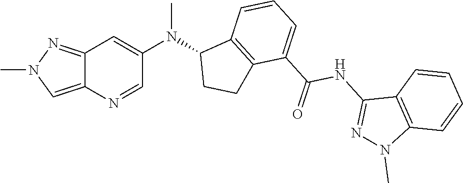 [SMILES: CN(c1cnc2cn(C)nc2c1)[C@H]1CCc2c(C(=O)Nc3nn(C)c4ccccc34)cccc21] | 452.2 | ¹H NMR (400 MHz, DMSO-d₆) δ 10.69 (s, 1H), 8.65 (s, 1H), 8.43 (s, 1H), 7.79- 7.74 (m, 2H), 7.62 (d, J = 8.5 Hz, 1H), 7.46-7.29 (m, 3H), 7.19-7.07 (m, 2H), 5.77-7.51 (m, 1H), 4.11 (s, 3H), 4.01 (s, 3H), 3.33-3.27 (m, 1H), 3.14-3.08 (m, 1H), 2.64 (s, 3H), 2.44-2.37 (m, 1H), 2.08-1.99 (m, 1H). |
| 80m | 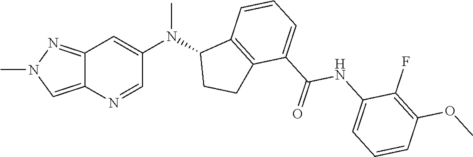 | 446.2 | ¹H NMR (400 MHz, DMSO-d₆) δ 9.99 (s, 1H), 8.64 (s, 1H), 8.43 (s, 1H), 7.67 (d, J = 7.3 Hz, 1H), 7.44-7.30 (m, 2H), 7.22 (t, J = 7.3 Hz, 1H), 7.16-7.10 (m, 2H), 7.04 (t, J = 8.0 Hz, 1H), 5.77-5.66 (m, 1H), 4.11 (s, 3H), 3.87 (s, 3H), 3.29-3.17 (m, 1H), 3.13-3.01 (m, 1H), 2.63 (s, 3H), 2.42-2.28 (m, 1H), 2.08-1.97 (m, 1H). |
| 80n | 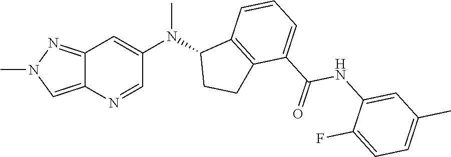 | 430.2 | ¹H NMR (400 MHz, DMSO-d₆) δ 9.96 (s, 1H), 8.64 (s, 1H), 8.43 (s, 1H), 7.66 (d, J = 7.3 Hz, 1H), 7.49 (d, J = 7.3 Hz, 1H), 7.40-7.30 (m, 2H), 7.22-7.12 (m, 2H), 7.11-7.03 (m, 1H), 5.76-5.65 (m, 1H), 4.11 (s, 3H), 3.32-3.20 (m, 1H), 3.15- 3.01 (m, 1H), 2.63 (s, 3H), 2.45-2.32 (m, 1H), 2.32 (s, 3H), 2.09-1.93 (m, 1H). |
| 80o | 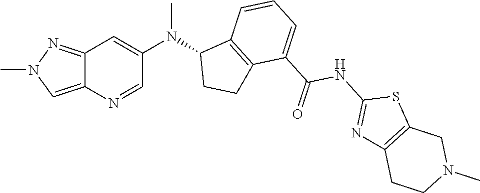 | 474.2 | ¹H NMR (400 MHz, DMSO-d₆) δ 12.39 (s, 1H), 8.63 (s, 1H), 8.43 (s, 1H), 7.82- 7.70 (m, 1H), 7.41-7.29 (m, 2H), 7.14 (s, 1H), 5.77-7.72 (m, 1H), 4.11 (s, 3H), 3.53 (s, 2H), 3.31-3.25 (m, 1H), 3.15- 3.04 (m, 1H), 2.74-2.68 (m, 4H), 2.61 (s, 3H), 2.39 (s, 3H), 2.39-2.31 (m, 1H), 2.07-1.97 (m, 1H). |
| 80p | 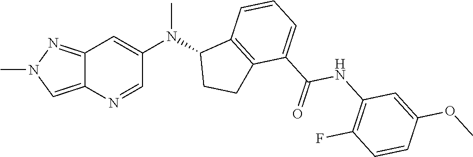 | 446.2 | ¹H NMR (400 MHz, DMSO-d₆) δ 10.00 (s, 1H), 8.64 (s, 1H), 8.43 (s, 1H), 7.67 (d, J = 7.1 Hz, 1H), 7.43-7.28 (m, 3H), 7.22 (t, J = 9.6 Hz, 1H), 7.14 (s, 1H), 6.87- 6.75 (m, 1H), 5.73-5.71 (m, 1H), 4.11 (s, 3H), 3.76 (s, 3H), 3.28-3.24 (m, 1H), 3.10-3.06 (m, 1H), 2.63 (s, 3H), 2.40- 2.36 (m, 1H), 2.11-1.95 (m, 1H). |
| 80q | 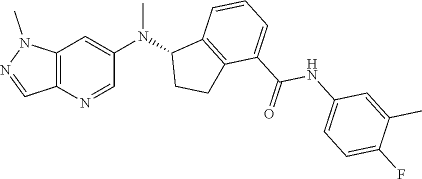 [SMILES: Cc1cc(NC(=O)c2cccc3c2CC[C@@H]3N(C)c2cnc3cnn(C)c3c2)ccc1F] | 430.2 | ¹H NMR (400 MHz, DMSO-d₆) δ 10.24 (s, 1H), 8.55 (s, 1H), 8.04 (s, 1H), 7.72- 7.70 (m, 1H), 7.63 (d, J = 7.4 Hz, 1H), 7.58-7.55 (m, 1H), 7.38 (t, J = 7.5 Hz, 1H), 7.35-7.29 (m, 2H), 7.12 (t, J = 9.2 Hz, 1H), 5.83-5.81 (m, 1H), 3.98 (s, 3H), 3.28-3.24 (m, 1H), 3.10-3.06 (m, 1H), 2.70 (s, 3H), 2.46-2.42 (m, 1H), 2.25 (s, 3H), 2.02-1.98 (m, 1H). |
| 80r | 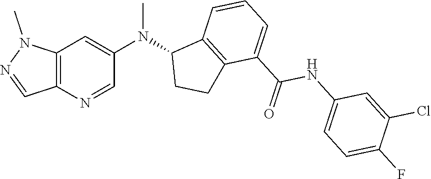 [SMILES: CN(c1cnc2cnn(C)c2c1)[C@H]1CCc2c(C(=O)Nc3ccc(F)c(Cl)c3)cccc21] | 450.1 | ¹H NMR (400 MHz, DMSO-d₆) δ 10.48 (s, 1H), 8.54 (s, 1H), 8.14-8.08 (m, 1H), 8.04 (s, 1H), 7.72-7.69 (m, 1H), 7.65 (d, J = 7.4 Hz, 1H), 7.45-7.40 (m, 2H), 7.36- 7.31 (m, 2H), 5.84-5.82 (m, 1H), 3.98 (s, 3H), 3.28-3.24 (m, 1H), 3.10-3.06 (m, 1H), 2.70 (s, 3H), 2.47-2.44 (m, 1H), 2.02-1.96 (m, 1H). |
| 80s | 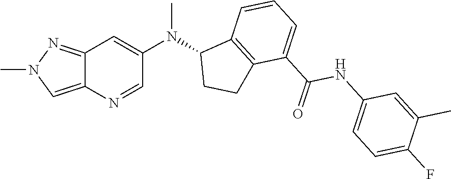 | 430.2 | ¹H NMR (400 MHz, DMSO-d₆) δ 10.23 (s, 1H), 8.63 (s, 1H), 8.43 (s, 1H), 7.75- 7.67 (m, 1H), 7.61 (d, J = 7.3 Hz, 1H), 7.60-7.51 (m, 1H), 7.41-7.29 (m, 2H), 7.17-7.08 (m, 2H), 5.78-5.67 (m, 1H), 4.11 (s, 3H), 3.27-3.15 (m, 1H), 3.13- 3.00 (m, 1H), 2.63 (s, 3H), 2.46-2.33 (m, 1H), 2.25 (s, 3H), 2.06-1.93 (m, 1H). |
| 80t | 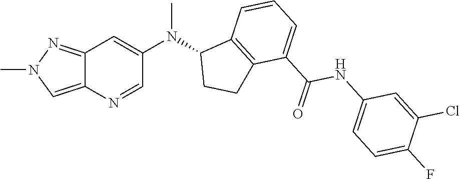 [SMILES: CN(c1cnc2cn(C)nc2c1)[C@H]1CCc2c(C(=O)Nc3ccc(F)c(Cl)c3)cccc21] | 450.1 | ¹H NMR (400 MHz, DMSO-d₆) δ 10.46 (s, 1H), 8.64 (s, 1H), 8.43 (s, 1H), 8.15- 8.03 (m, 1H), 7.74-7.66 (m, 1H), 7.64 (d, J = 7.3 Hz, 1H), 7.48-7.33 (m, 3H), 7.14 (d, J = 2.3 Hz, 1H), 5.77-5.68 (m, 1H), 4.11 (s, 3H), 3.28-3.17 (m, 1H), 3.14- 3.01 (m, 1H), 2.63 (s, 3H), 2.43-2.36 (m, 1H), 2.07-1.96 (m, 1H). |
| 80u | 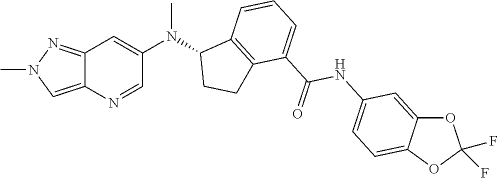 [SMILES: CN(c1cnc2cn(C)nc2c1)[C@H]1CCc2c(C(=O)Nc3ccc4c(c3)OC(F)(F)O4)cccc21] | 478.2 | ¹H NMR (400 MHz, DMSO-d₆) δ 10.48 (s, 1H), 8.64 (s, 1H), 8.43 (s, 1H), 7.92 (s, 1H), 7.63 (d, J = 7.2 Hz, 1H), 7.49 (d, J = 8.8 Hz, 1H), 7.41-7.33 (m, 3H), 7.14 (s, 1H), 5.73-5.71 (m, 1H), 4.11 (s, 3H), 3.24-3.20 (m, 1H), 3.13-3.02 (m, 1H), 2.62 (s, 3H), 2.40-2.37 (m, 1H), 2.03- 2.00 (m, 1H). |
| 80v | 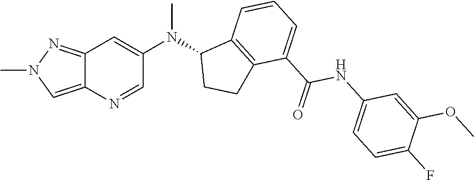 | 446.2 | ¹H NMR (400 MHz, DMSO-d₆) δ 10.28 (s, 1H), 8.64 (s, 1H), 8.43 (s, 1H), 7.68- 7.62 (m, 2H), 7.38-7.30 (m, 3H), 7.20- 7.12 (m, 2H), 5.74-5.70 (m, 1H), 4.11 (s, 3H), 3.84 (s, 3H), 3.26-3.19 (m, 1H), 3.12-3.03 (m, 1H), 2.63 (s, 3H), 2.44- 2.35 (m, 1H), 2.06-1.97 (m, 1H). |
| 80w | 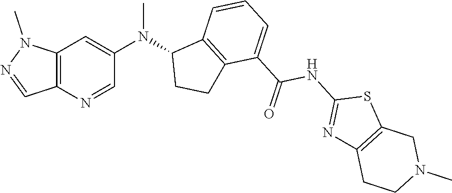 [SMILES: CN1CCc2nc(NC(=O)c3cccc4c3CC[C@@H]4N(C)c3cnc4cnn(C)c4c3)sc2C1] | 474.2 | ¹H NMR (400 MHz, DMSO-d₆) δ 12.42 (s, 1H), 8.54 (s, 1H), 8.03 (s, 1H), 7.81- 7.77 (m, 1H), 7.38-7.33 (m, 3H), 5.84- 5.80 (m, 1H), 3.98 (s, 3H), 3.55 (s, 2H), 3.31-3.28 (m, 1H), 3.15-3.07 (m, 1H), 2.74-2.71 (m, 4H), 2.69 (s, 3H), 2.49- 2.43 (m, 1H), 2.40 (s, 3H), 2.05-1.98 (m, 1H). |
| 80x | 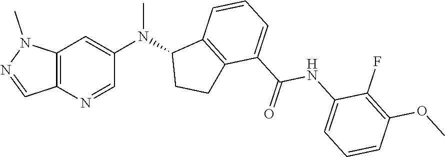 [SMILES: COc1cccc(NC(=O)c2cccc3c2CC[C@@H]3N(C)c2cnc3cnn(C)c3c2)c1F] | 446.2 | ¹H NMR (400 MHz, DMSO-d₆) δ 10.06 (s, 1H), 8.24 (s, 1H), 8.15 (s, 1H), 7.90 (s, 1H), 7.77 (d, J = 7.6 Hz, 1H), 7.67 (d, J = 7.5 Hz, 1H), 7.43 (t, J = 7.6 Hz, 1H), 7.23 (t, J = 7.2 Hz, 1H), 7.14 (t, J = 8.2 Hz, 1H), 7.04 (t, J = 7.9 Hz, 1H), 6.10-6.02 (m, 1H), 4.07 (s, 3H), 3.87 (s, 3H), 3.34-3.27 (m, 1H), 3.22-3.11 (m, 1H), 2.74-2.65 (m, 1H), 2.19-2.09 (m, 1H). |
| 80y | 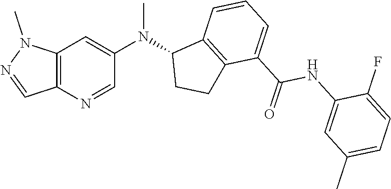 [SMILES: Cc1ccc(F)c(NC(=O)c2cccc3c2CC[C@@H]3N(C)c2cnc3cnn(C)c3c2)c1] | 430.2 | ¹H NMR (400 MHz, DMSO-d₆) δ 9.97 (s, 1H), 8.55 (s, 1H), 8.04 (s, 1H), 7.68 (d, J = 7.4 Hz, 1H), 7.50 (d, J = 7.4 Hz, 1H), 7.38 (t, J = 7.5 Hz, 1H), 7.34-7.32 (m, 2H), 7.20-7.15 (m, 1H), 7.09-7.03 (m, 1H), 5.83-5.81 (m, 1H), 3.98 (s, 3H), 3.28-3.24 (m, 1H), 3.12-3.08 (, 1H), 2.70 (s, 3H), 2.46-2.42 (m, 1H), 2.32 (s, 3H), 2.03-1.98 (m, 1H). |

### Example 81

### (S)-3-cyclopropyl-N-(1-((2-methyl-2H-pyrazolo[4,3-b]pyridin-6-yl)amino)-2,3-dihydro-1H-inden-4-yl)benzamide

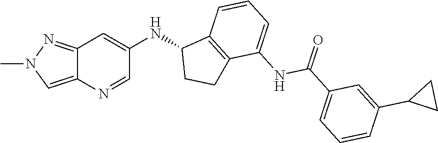 [SMILES: Cn1cc2ncc(N[C@H]3CCc4c(NC(=O)c5cccc(C6CC6)c5)cccc43)cc2n1]

Compound 81 was prepared by the following steps:

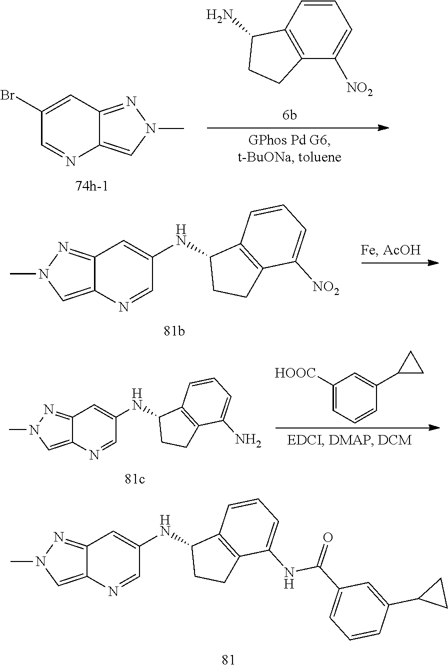 [SMILES: N[C@H]1CCc2c1cccc2[N+](=O)[O-] ; Cn1cc2ncc(Br)cc2n1 ; Cn1cc2ncc(N[C@H]3CCc4c3cccc4[N+](=O)[O-])cc2n1 ; Cn1cc2ncc(N[C@H]3CCc4c(N)cccc43)cc2n1 ; O=C(O)c1cccc(C2CC2)c1 ; Cn1cc2ncc(N[C@H]3CCc4c(NC(=O)c5cccc(C6CC6)c5)cccc43)cc2n1]

Step 1: Compound 74h-1 (2.8 g, 13.2 mmol), compound 6b (2.8 g, 15.9 mmol), sodium tert-butoxide (1.78 g, 18.5 mmol) and GPhos Pd G6 (1.25 g, 1.32 mmol) were dissolved in toluene (28 mL). The resulting mixture was degassed with N₂ for three times, then reacted at 100° C. for 30 h. The reaction was complete detected by TLC. Water was added into the reaction mixture, and the mixture was extracted with EtOAc. The combined organic phases were sequentially washed with water and brine, dried over anhydrous Na₂SO₄ and filtered. After concentration, the residue was purified on an ISCO chromatography to afford compound 81b (3.3 g, yield: 82.5%) as a solid. ESI-MS (M/S): 310.1 [M+H]⁺.

Step 2: A mixture of Compound 81b (3.3 g, 10.87 mmol) and ferrous powder (3.65 g, 65.23 mmol) in AcOH (11 mL) was stirred at 60° C. for 2 h. The reaction was complete detected by TLC. The reaction mixture was diluted with water and adjusted to pH=7-8 with saturated aqueous NaHCO₃, then extracted with EtOAc. The combined organic phases were sequentially washed with water and brine, dried over anhydrous Na₂SO₄ and filtered. After concentration, the residue was purified on an ISCO chromatography to afford compound 81c (1.8 g, yield: 60.0%) as a solid. ESI-MS (M/S): 280.2 [M+H]⁺.

Step 3: Similar procedure to step 2 of example 12 was followed. ESI-MS (M/S): 424.2 [M+H]⁺. ¹H NMR (62 MHz, acetone-d₆) δ 9.09 (s, 1H), 8.42-8.00 (m, 2H), 7.94-7.47 (m, 3H), 7.47-7.11 (m, 4H), 7.07-6.84 (m, 1H), 5.79-5.43 (m, 1H), 5.36-4.82 (m, 1H), 4.11 (s, 3H), 3.38-2.87 (m, 2H), 2.75-2.42 (m, 1H), 1.91-1.68 (m, 1H), 1.39-0.60 (m, 5H).

### Example 82

### (S)-2-fluoro-3-methyl-N-(1-((2-methyl-2H-pyrazolo[4,3-b]pyridin-6-yl)amino)-2,3-dihydro-1H-inden-4-yl)benzamide

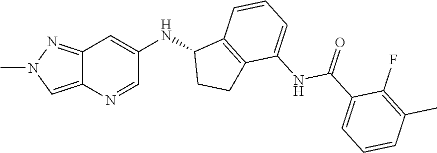

Compound 82 was prepared by the following steps:

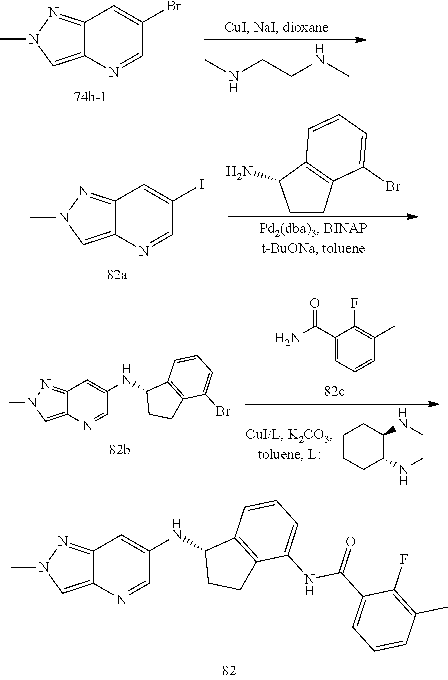

Step 1: To a 100 mL two-necked round bottom flask was sequentially added compound 74h-1 (2500 mg, 11.8 mmol), NaI (5299 mg, 35.4 mmol), CuI (229 mg, 1.2 mmol), N,N′-dimethyl-1,2-ethanediamine (212 mg, 2.4 mmol) and 1,4-dioxane (12 mL). The mixture was degassed with N₂ for three times with stirring, then heated to 110° C. and stirred for about 12 h. The reaction was complete detected by LC-MS. After natural cooling to 30° C., CH₂Cl₂ (150 mL) and water (100 mL) was added into the reaction mixture. The mixture was stirred for a moment and the separated aqueous layer was discarded. The organic layer was washed with 13% ammonium hydroxide (100 mL), dried over anhydrous Na₂SO₄, and concentrated to dryness under reduced pressure. The residue was purified on an ISCO chromatography to afford compound 82a (2.3 g, yield: 75.4%). ESI-MS (M/S): 260.0 [M+H]⁺.

Step 2: To a 20 mL vial was sequentially added compound 82a (777 mg, 3.0 mmol), (S)-4-Bromo-2,3-dihydro-1H-inden-1-amine (424 mg, 2.0 mmol), Sodium tert-butoxide (384 mg, 4.0 mmol), BINAP (249 mg, 0.4 mmol), Pd₂(dba); (46 mg, 0.05 mmol) and toluene (3 mL). The mixture was stirred well and degassed with N₂ for three times, then heated to 100° C. and stirred for about 3 h. The reaction was complete detected by TLC. After natural cooling to 30° C., CH₂Cl₂ (20 mL) and water (20 mL) was added into the reaction mixture. The resulting mixture was stirred for a moment and the separated aqueous layer was discarded. The organic layer was dried over anhydrous Na₂SO₄ and concentrated to dryness under reduced pressure. The resulting residue was purified on an ISCO chromatography to afford compound 82b (226 mg, yield: 33.0%). ESI-MS (M/S): 342.1 [M+H]⁺.

Step 3: Similar procedure to step 2 of example 77 was followed. ESI-MS (M/S): 416.2 [M+H]⁺. ¹H NMR (400 MHz, DMSO-d₆) δ 9.95 (s, 1H), 8.32 (s, 1H), 8.18 (s, 1H), 7.58-7.45 (m, 3H), 7.28-7.14 (m, 3H), 6.89 (s, 1H), 6.41 (d, J=8.0 Hz, 1H), 5.06 (d, J=7.4 Hz, 1H), 4.05 (s, 3H), 3.06-2.94 (m, 1H), 2.88-2.84 (m, 1H), 2.63-2.56 (m, 1H), 2.32 (s, 3H), 1.88-1.82 (m, 1H).

### Example 83

### (S)-2-fluoro-N-(7-fluoro-1-((2-methyl-2H-pyrazolo[4,3-b]pyridin-6-yl)amino)-2,3-dihydro-1H-inden-4-yl)-3-methylbenzamide

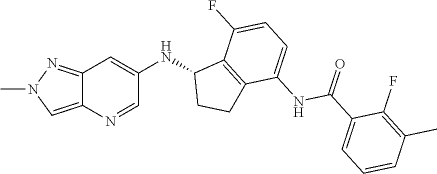

The preparation of compound 83 refer to the synthesis of compound 82 in example 82.

ESI-MS (M/S): 434.2 [M+H]⁺. ¹H NMR (400 MHz, DMSO-d₆) δ 9.99 (s, 1H), 8.31 (s, 1H), 8.11 (s, 1H), 7.61-5.57 (m, 1H), 7.54-7.45 (m, 2H), 7.23 (t, J=7.6 Hz, 1H), 7.06 (t, J=8.8 Hz, 1H), 6.83 (s, 1H), 6.46 (d, J=7.7 Hz, 1H), 5.21 (d, J=8.6 Hz, 1H), 4.05 (s, 3H), 3.09-3.04 (m, 1H), 2.98-2.86 (m, 1H), 2.46-2.42 (m, 1H), 2.32 (s, 3H), 2.04-2.00 (m, 1H).

### Example 84

### (S)-7-fluoro-N-(2-fluoro-3-methylphenyl)-1-((2-methyl-2H-pyrazolo[4,3-b]pyridin-6-yl)amino)-2,3-dihydro-1H-indene-4-carboxamide

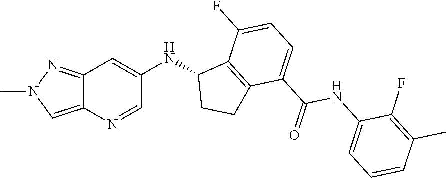

Compound 84 was prepared by the following steps:

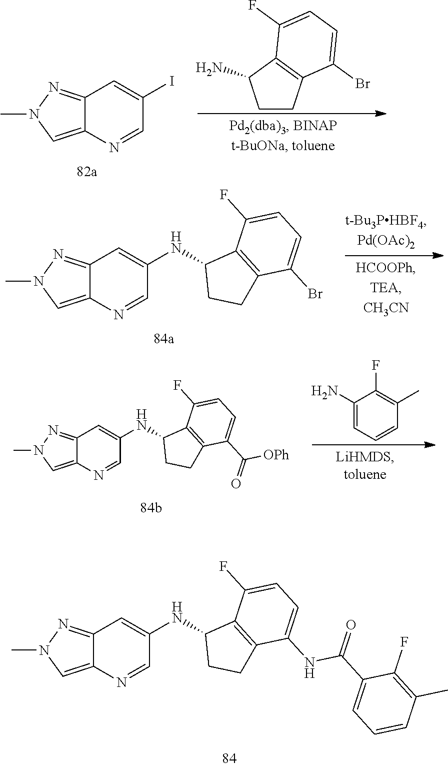

Step 1: The preparation of compound 84a refer to the synthesis of compound 82b in example 82.

Step 2 and step 3: Similar procedure to the synthesis of compound 50 in example 50 was followed.

Compound 84: ESI-MS (M/S): 434.2 [M+H]⁺. ¹H NMR (400 MHz, DMSO-d₆) δ 9.98 (s, 1H), 8.32 (s, 1H), 8.10 (s, 1H), 7.81-7.79 (m, 1H), 7.48 (t, J=7.5 Hz, 1H), 7.25-7.07 (m, 3H), 6.87 (s, 1H), 6.43 (d, J=7.8 Hz, 1H), 5.23-5.19 (m, 1H), 4.06 (s, 3H), 3.33-3.30 (m, 1H), 3.20-3.16 (m, 1H), 2.44-2.40 (m, 1H), 2.28 (s, 3H), 2.06-2.02 (m, 1H).

### Example 85

### (S)—N-(3-cyclopropylphenyl)-7-fluoro-1-((1-methyl-1H-pyrazolo[4,3-b]pyridin-6-yl)oxy)-2,3-dihydro-1H-indene-4-carboxamide

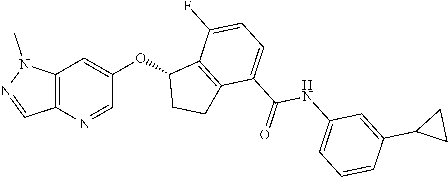 [SMILES: Cn1ncc2ncc(O[C@H]3CCc4c(C(=O)Nc5cccc(C6CC6)c5)ccc(F)c43)cc21]

Compound 85 was prepared by the following steps:

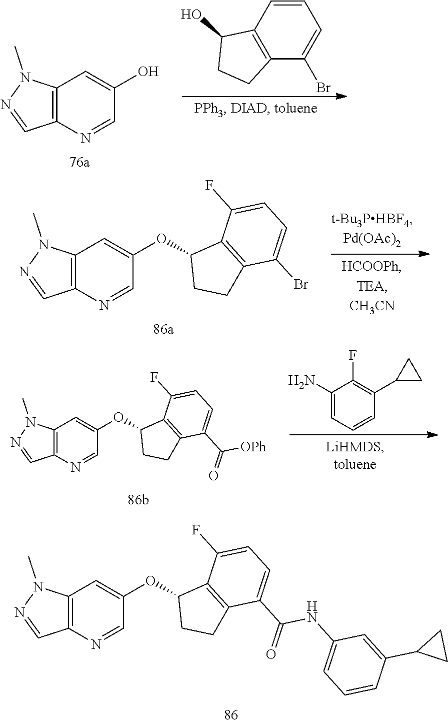 [SMILES: Cn1ncc2ncc(O)cc21 ; O[C@@H]1CCc2c(Br)cccc21 ; Cn1ncc2ncc(O[C@H]3CCc4c(Br)ccc(F)c43)cc21 ; Cn1ncc2ncc(O[C@H]3CCc4c(C(=O)Oc5ccccc5)ccc(F)c43)cc21 ; Nc1cccc(C2CC2)c1F ; Cn1ncc2ncc(O[C@H]3CCc4c(C(=O)Nc5cccc(C6CC6)c5)ccc(F)c43)cc21]

Step 1: The preparation of compound 85a refer to the synthesis of compound 77a in example 77.

Step 2 and step 3: Similar procedure to the synthesis of compound 53 in example 53 was followed.

Compound 85: ESI-MS (M/S): 443.2 [M+H]⁺. ¹H NMR (400 MHz, DMSO-d₆) δ 10.19 (s, 1H), 8.22 (s, 1H), 8.16 (s, 1H), 7.90-7.79 (m, 2H), 7.53 (d, J=8.2 Hz, 1H), 7.48 (s, 1H), 7.30-7.20 (m, 2H), 6.84 (d, J=7.7 Hz, 1H), 6.24 (d, J=6.1 Hz, 1H), 4.06 (s, 3H), 3.47-3.38 (m, 1H), 3.24-3.20 (m, 1H), 2.65-2.62 (m, 2H), 2.32-2.20 (m, 1H), 1.93-1.89 (m, 1H), 1.01-0.92 (m, 2H), 0.70-0.61 (m, 2H).

### Example 86

### (S)—N⁴-(5-(tert-butyl) isoxazol-3-yl)-N₁-(1-methyl-1H-pyrazolo[3,4-b]pyrazin-6-yl)-2,3-dihydro-1H-indene-1,4-diamine

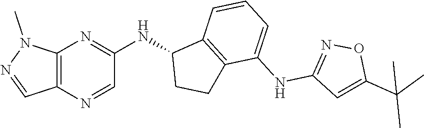 [SMILES: Cn1ncc2ncc(N[C@H]3CCc4c(Nc5cc(C(C)(C)C)on5)cccc43)nc21]

Compound 86 was prepared by the following steps:

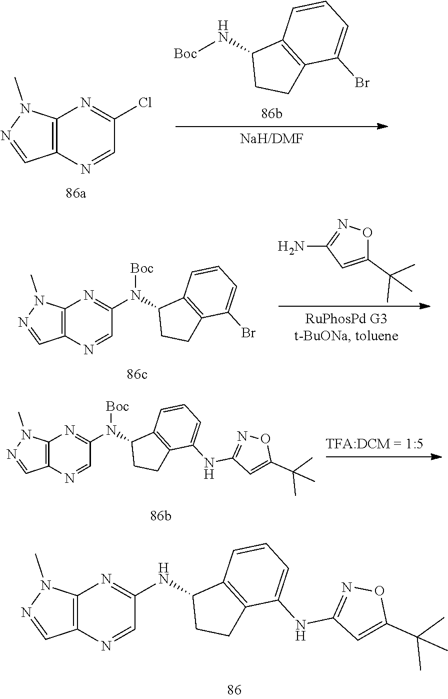

Step 1: To a solution of compound 86b (200 mg, 0.64 mmol) in DMF (0.6 mL) was added 60% NaH (33.2 mg, 0.83 mmol) at 0° C., followed by addition of compound 86a (98 mg, 0.58 mmol) after stirring for 15 min. The resulting mixture was allowed to warm to room temperature and stirred for 3 h. The reaction was complete detected by TLC. The reaction mixture was quenched by addition of water and extracted with EtOAc. The combined organic layers were washed with water, saturated aqueous NaHCO₃ and brine in turn, dried over anhydrous Na₂SO₄ and filtered. After concentration, the residue was purified on an ISCO chromatography to afford compound 86c (218 mg, yield: 76.8%). ESI-MS (m/z): 445.3 [M+1]⁺.

Step 2: A solution of compound 86c (218 mg, 0.49 mmol), 3-amino-5-tert-butylisoxazole (103 mg, 0.74 mmol), RuPhos Pd G3 (41 mg, 0.05 mmol) and t-BuONa (95 mg, 0.98 mmol) in toluene (1 mL) was heated to 100° C. and stirred for 3 h. The reaction was complete detected by TLC. After cooling to room temperature, the reaction mixture was quenched with water and extracted with EtOAc. The combined organic layers were washed with water, saturated aqueous NaHCO₃ and brine in turn, dried over anhydrous Na₂SO₄ and filtered. After concentration, the residue was purified on an ISCO chromatography to afford compound 86d (120 mg, yield: 48.5%). ESI-MS (m/z): 504.6 [M+1]⁺.

Step 3: To a solution of compound 86d (120 mg, 0.24 mmol) in DCM (2 mL) was added TFA (10 mL). The resulting mixture was stirred at room temperature for 2 h. The reaction was complete detected by TLC. After concentration under reduced pressure, the mixture was diluted with CH₂Cl₂ and washed sequentially with 5% aqueous NaOH, water and brine, then dried over anhydrous Na₂SO₄ and filtered. After concentration, the residue was purified on an ISCO chromatography to afford compound 86 (55 mg, yield: 85.8%). ESI-MS (m/z): 404.5 [M+1]⁺. ¹H NMR (62 MHz, acetone-d₆) δ 8.12-6.77 (m, 7H), 5.93 (s, 1H), 5.84-5.51 (m, 1H), 3.91 (s, 3H), 3.21-2.57 (m, 2H), 2.42-1.76 (m, 2H), 1.32 (s, 9H).

### Example 87

### (S)—N′-(1-methyl-1H-pyrazolo[3,4-b]pyrazin-6-yl)-N⁴-(5-(1,1,1-trifluoro-2-methylpropan-2-yl)isoxazol-3-yl)-2,3-dihydro-1H-indene-1,4-diamine

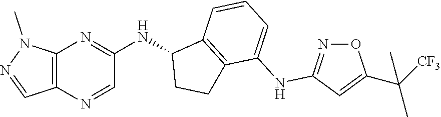 [SMILES: Cn1ncc2ncc(N[C@H]3CCc4c(Nc5cc(C(C)(C)C(F)(F)F)on5)cccc43)nc21]

The preparation of compound 87 refer to the synthesis of compound 86 in example 86.

ESI-MS (m/z): 458.2 [M+1]⁺. ¹H NMR (62 MHz, acetone-d₆) δ 8.15-7.59 (m, 4H), 7.42-6.84 (m, 3H), 6.29 (s, 1H), 5.99-5.49 (m, 1H), 3.91 (s, 3H), 3.25-2.85 (m, 2H), 2.77-2.16 (m, 1H), 1.97-1.69 (m, 1H), 1.67-1.49 (m, 6H).

### Example 88

### (S)—N-(3-cyclopropylphenyl)-1-((1-methyl-1H-pyrazolo[3,4-b]pyrazin-6-yl)amino)-2,3-dihydro-1H-indene-4-carboxamide

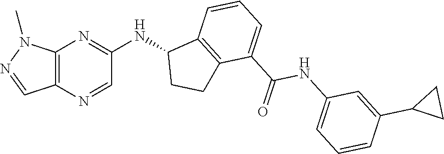 [SMILES: Cn1ncc2ncc(N[C@H]3CCc4c(C(=O)Nc5cccc(C6CC6)c5)cccc43)nc21]

Compound 88 was prepared by the following steps:

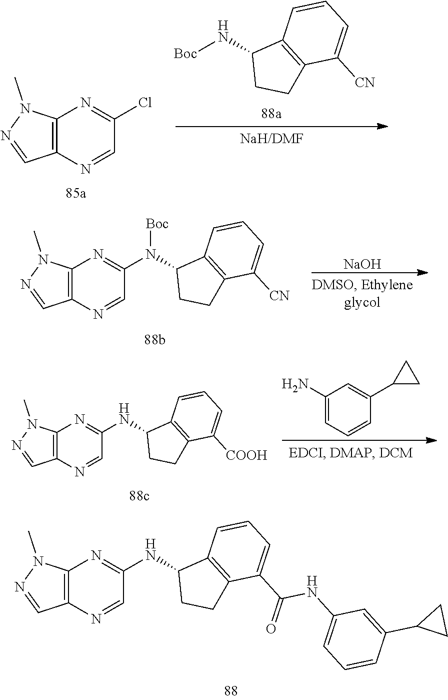

The preparation of compound 88 refer to the synthesis of compound 26 in example 26.

ESI-MS (M/S): 425.2 [M+H]⁺. ¹H NMR (62 MHz, Acetone-d₆) δ 9.27 (s, 1H), 8.00 (s, 1H), 7.87 (s, 1H), 7.78-7.03 (m, 7H), 6.85 (d, J=7.8 Hz, 1H), 6.03-5.49 (m, 1H), 3.92 (s, 3H), 3.52-3.04 (m, 2H), 2.76-2.21 (m, 1H), 1.93-1.66 (m, 1H), 1.50-1.06 (m, 1H), 1.05-0.47 (m, 4H).

### Example 89

### (S)—N₁-(2-methyl-2H-pyrazolo[4,3-b]pyridin-6-yl)-N⁴-(5-(1-methylcyclopropyl) isoxazol-3-yl)-2,3-dihydro-1H-indene-1,4-diamine

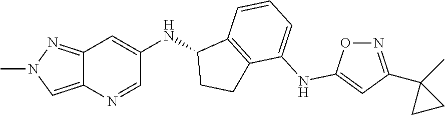 [SMILES: Cn1cc2ncc(N[C@H]3CCc4c(Nc5cc(C6(C)CC6)no5)cccc43)cc2n1]

The synthetic method of compound 89 is similar to that of example 66, which is prepared by the following steps:

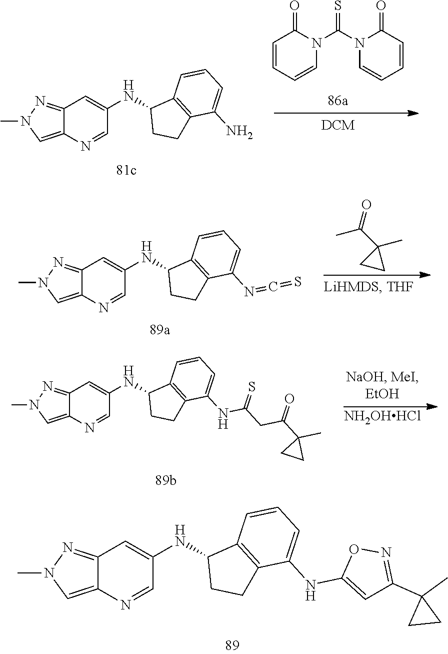

Step 1: To a solution of compound 81c (2.79 g, 10.0 mmol) in CH₂Cl₂ (60 mL) was added compound 66a (2.32 g, 10.0 mmol). The resulting mixture was stirred at room temperature for 60 min. The reaction was complete detected by TLC. The reaction mixture was washed with water (60 mL), dried over anhydrous Na₂SO₄, filtered and concentrated to give compound 89a (3.05 g, yield: 95.0%) as yellow solid. ESI-MS (m/z): 322.1 [M+1]⁺.

Step 2: To a stirred solution of methyl 1-methylcyclopropyl ketone (196 mg, 2.0 mmol) in THF (5 mL) was added LiHMDS (2 mL, 1.0 mol/L) at 0° C., followed by addition of a solution of compound 89a (642 mg, 2.0 mmol) in THF (5 mL) after 1 h. The resulting mixture was continued to stir at 0° C. for 2 h. The reaction mixture was quenched with saturated aqueous NHCl (5 mL) and extracted with EtOAc (20 mL*2). The combined organic phases were washed with water, dried over anhydrous Na₂SO₄ and concentrated to give the solid product 89b (213 mg, yield: 25.4%). ESI-MS (m/z): 420.2 [M+1]⁺.

Step 3: To a solvent of EtOH (10 mL) was added compound 89b (210 mg, 0.5 mmol) and NaOH (200 mg, 5.0 mmol) in turn, followed by addition of CH₃I (106 mg, 0.55 mmol) at −20° C. Then hydroxylamine hydrochloride (70 mg, 1.0 mmol) was added to the above mixture after 1 h. The resulting mixture was heated to 80° C. and stirred for 1 h. After cooling to room temperature, water (20 mL) was added, and the mixture was extracted with EtOAc (20 mL*2). The combined organic phases were dried over anhydrous Na₂SO₄ and concentrated. The residue was purified on an ISCO chromatography to afford the solid product 89 (66 mg, yield: 33.0%). ESI-MS (m/z): 401.3 [M+1]⁺. ¹H NMR (400 MHz, DMSO-d₆) δ 9.18 (s, 1H), 8.31 (s, 1H), 8.15 (d, J=2.4 Hz, 1H), 7.22-7.13 (m, 2H), 7.04-6.96 (m, 1H), 6.85 (d, J=2.4 Hz, 1H), 6.37 (d, J=8.1 Hz, 1H), 5.28 (s, 1H), 5.06-4.96 (m, 1H), 4.04 (s, 3H), 3.01-2.88 (m, 1H), 2.84-2.72 (m, 1H), 2.64-2.56 (m, 1H), 1.90-1.76 (m, 1H), 1.35 (s, 3H), 0.98-0.91 (m, 2H), 0.83-0.74 (m, 2H).

### Example 90

### (S)—N⁴-(5-(tert-butyl) isoxazol-3-yl)-N¹-(2-methyl-2H-pyrazolo[4,3-b]pyridin-6-yl)-2,3-dihydro-1H-indene-1,4-diamine

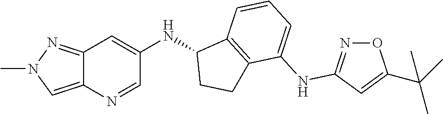 [SMILES: Cn1cc2ncc(N[C@H]3CCc4c(Nc5cc(C(C)(C)C)on5)cccc43)cc2n1]

Compound 90 was prepared by the following steps:

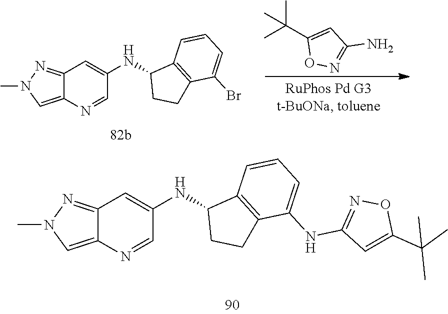

The preparation of compound 90 refer to the synthesis of compound 41 in example 41.

ESI-MS (m/z): 403.2 [M+1]⁺. ¹H NMR (62 MHz, acetone-d₆) δ 8.35-6.78 (m, 7H), 5.66-5.32 (m, 1H), 5.32-4.85 (m, 1H), 4.11 (s, 3H), 3.39-2.87 (m, 2H), 2.75-2.22 (m, 1H), 1.97-1.69 (m, 1H), 1.32 (s, 9H).

### Example 91

The preparation of compounds 91a-91s refer to the synthesis of compound 89 in example 89 or compound 90 in example 90.

TABLE 23

| | | MS | |
|---|---|---|---|
| Cpds | Structure | [M + 1]⁺ | ¹HNMR |
| 91a | 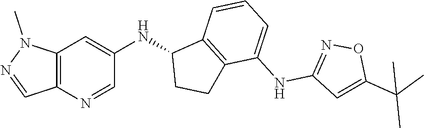 [SMILES: Cn1ncc2ncc(N[C@H]3CCc4c(Nc5cc(C(C)(C)C)on5)cccc43)cc21] | 403.2 | ¹H NMR (400 MHz, DMSO-d₆) δ 8.31 (s, 1H), 8.11 (d, J = 2.3 Hz, 1H), 7.96- 7.89 (m, 1H), 7.78 (d, J = 8.1 Hz, 1H), 7.17 (t, J = 7.8 Hz, 1H), 7.13-7.07 (m, 1H), 6.91 (d, J = 7.4 Hz, 1H), 6.64 (d, J = 8.4 Hz, 1H), 5.99 (s, 1H), 5.15-5.04 (m, 1H), 3.92 (s, 3H), 3.07-2.95 (m, 1H), 2.88-2.75 (m, 1H), 2.71-2.61 |
| | | | (m, 1H), 1.92-1.75 (m, 1H), 1.29 (s, |
| | | | 9H). |
| 91b | 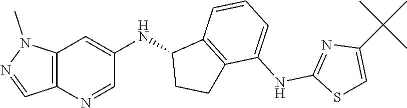 [SMILES: Cn1ncc2ncc(N[C@H]3CCc4c(Nc5nc(C(C)(C)C)cs5)cccc43)cc21] | 419.2 | ¹H NMR (62 MHz, acetone-d₆) δ 9.40 (s, 1H), 8.26-7.82 (m, 3H), 7.38-6.47 (m, 4H), 6.40 (s, 1H), 5.35-4.89 (m, 1H), 3.93 (s, 3H), 3.19-2.62 (m, 3H), 2.31-1.51 (m, 1H), 1.27 (s, 9H). |
| 91c | 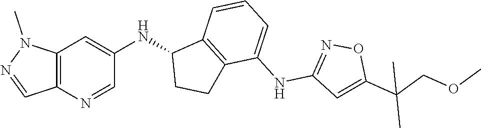 [SMILES: COCC(C)(C)c1cc(Nc2cccc3c2CC[C@@H]3Nc2cnc3cnn(C)c3c2)no1] | 433.2 | ¹H NMR (62 MHz, acetone-d₆) δ 8.26- 7.74 (m, 3H), 7.54 (s, 1H), 7.38-6.81 (m, 3H), 5.97 (s, 1H), 5.90-5.65 (m, 1H), 5.38-4.92 (m, 1H), 3.95 (s, 3H), 3.43 (s, 2H), 3.30 (s, 3H), 3.17-2.83 (m, 3H), 2.69-2.25 (m, 1H), 1.30 (s, 6H). |
| 91d | 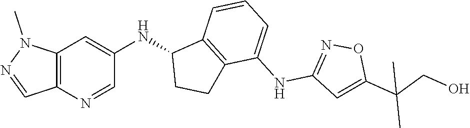 [SMILES: Cn1ncc2ncc(N[C@H]3CCc4c(Nc5cc(C(C)(C)CO)on5)cccc43)cc21] | 419.2 | ¹H NMR (62 MHz, acetone-d₆) δ 8.33- 6.79 (m, 7H), 5.99 (s, 1H), 5.93-5.61 (m, 1H), 5.39-4.96 (m, 1H), 3.95 (s, 3H), 3.61 (s, 2H), 3.16-2.90 (m, 2H), 3.15-2.82 (m, 3H), 2.68-2.24 (m, 1H), 1.29 (s, 6H). |
| 91e | 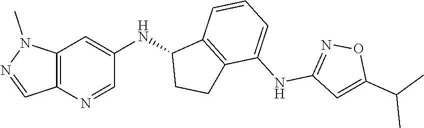 [SMILES: CC(C)c1cc(Nc2cccc3c2CC[C@@H]3Nc2cnc3cnn(C)c3c2)no1] | 389.2 | ¹H NMR (62 MHz, acetone-d₆) δ 8.30- 7.76 (m, 3H), 7.59 (s, 1H), 7.40-6.81 (m, 3H), 6.03-5.65 (m, 2H), 5.13 (q, J = 7.0 Hz, 1H), 3.95 (s, 3H), 3.38-2.92 (m, 2H), 2.85-2.13 (m, 2H), 1.95-1.59 (m, 1H), 1.33 (s, 3H), 1.21 (s, 3H). |
| 91f | 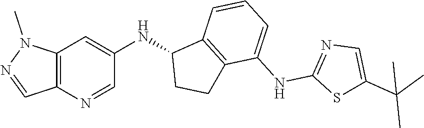 [SMILES: Cn1ncc2ncc(N[C@H]3CCc4c(Nc5ncc(C(C)(C)C)s5)cccc43)cc21] | 419.2 | ¹H NMR (62 MHz, acetone-d₆) δ 8.42 (s, 1H), 8.28-7.97 (m, 2H), 7.85 (d, J = 0.9 Hz, 1H), 7.15 (d, J = 7.9 Hz, 3H), 6.93 (s, 1H), 5.79 (d, J = 8.3 Hz, 1H), 5.37-4.93 (m, 1H), 3.96 (s, 3H), 3.23-2.85 (m, 2H), 2.70-2.22 (m, 1H), 1.34 (s, 9H). |
| 91g | 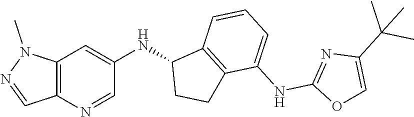 [SMILES: Cn1ncc2ncc(N[C@H]3CCc4c(Nc5nc(C(C)(C)C)co5)cccc43)cc21] | 403.2 | ¹H NMR (62 MHz, acetone-d₆) δ 8.41- 7.98 (m, 3H), 7.95-7.76 (m, 1H), 7.42- 6.87 (m, 4H), 5.98-5.64 (m, 1H), 5.40- 4.95 (m, 1H), 3.96 (s, 3H), 3.26-2.92 (m, 2H), 2.72-2.42 (m, 1H), 1.95-1.74 (m, 1H), 1.25 (s, 9H). |
| 91h | 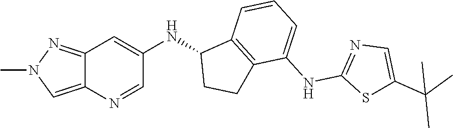 [SMILES: Cn1cc2ncc(N[C@H]3CCc4c(Nc5ncc(C(C)(C)C)s5)cccc43)cc2n1] | 419.2 | ¹H NMR (62 MHz, methanol-d₄) δ 8.14 (d, J = 2.7 Hz, 2H), 7.73-6.75 (m, 5H), 5.05 (s, 1H), 4.12 (s, 3H), 3.10-1.89 (m, 4H), 1.34 (s, 9H). |
| 9i | 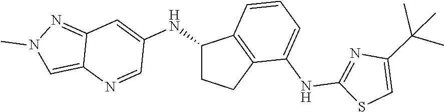 [SMILES: Cn1cc2ncc(N[C@H]3CCc4c(Nc5nc(C(C)(C)C)cs5)cccc43)cc2n1] | 419.2 | ¹H NMR (62 MHz, acetone-d₆) δ 8.71- 8.36 (m, 1H), 8.35-8.03 (m, 3H), 7.42- 6.85 (m, 3H), 6.37 (s, 1H), 5.56-4.86 (m, 2H), 4.11 (s, 3H), 3.21-2.91 (m, 2H), 2.73-2.21 (m, 1H), 1.92-1.75 (m, 1H), 1.31 (s, 9H). |
| 9j | 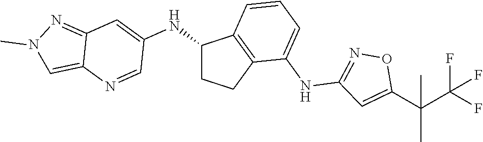 [SMILES: Cn1cc2ncc(N[C@H]3CCc4c(Nc5cc(C(C)(C)C(F)(F)F)on5)cccc43)cc2n1] | 457.2 | ¹H NMR (62 MHz, methanol-d₄) δ 8.23- 8.04 (m, 2H), 7.85-7.55 (m, 1H), 7.38- 6.79 (m, 3H), 6.22 (s, 1H), 5.18-4.91 (m, 1H), 4.11 (s, 3H), 3.11-1.87 (m, 4H), 1.57 (s, 6H). |
| 91k | 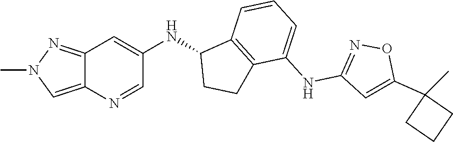 [SMILES: Cn1cc2ncc(N[C@H]3CCc4c(Nc5cc(C6(C)CCC6)on5)cccc43)cc2n1] | 415.2 | \ |
| 91l | 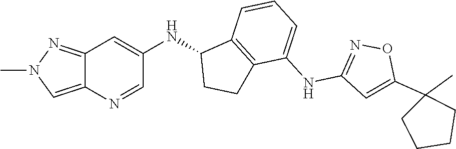 [SMILES: Cn1cc2ncc(N[C@H]3CCc4c(Nc5cc(C6(C)CCCC6)on5)cccc43)cc2n1] | 429.2 | \ |
| 91m | 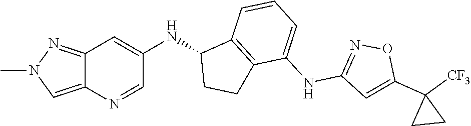 [SMILES: Cn1cc2ncc(N[C@H]3CCc4c(Nc5cc(C6(C(F)(F)F)CC6)on5)cccc43)cc2n1] | 455.2 | \ |
| 91n | 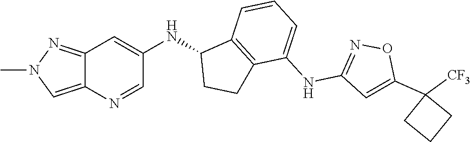 [SMILES: Cn1cc2ncc(N[C@H]3CCc4c(Nc5cc(C6(C(F)(F)F)CCC6)on5)cccc43)cc2n1] | 469.2 | \ |
| 91o | 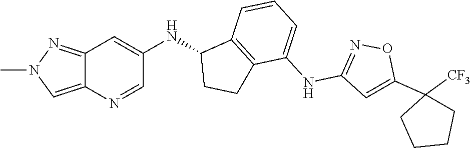 [SMILES: Cn1cc2ncc(N[C@H]3CCc4c(Nc5cc(C6(C(F)(F)F)CCCC6)on5)cccc43)cc2n1] | 483.2 | \ |
| 91p | 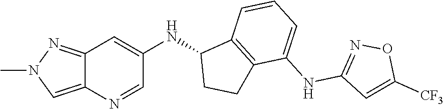 [SMILES: Cn1cc2ncc(N[C@H]3CCc4c(Nc5cc(C(F)(F)F)on5)cccc43)cc2n1] | 469.2 | \ |
| 91q | 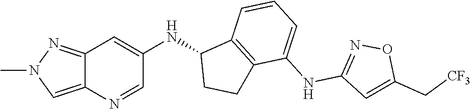 [SMILES: Cn1cc2ncc(N[C@H]3CCc4c(Nc5cc(CC(F)(F)F)on5)cccc43)cc2n1] | 415.1 | \ |
| 91r | 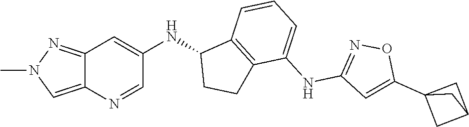 [SMILES: Cn1cc2ncc(N[C@H]3CCc4c(Nc5cc(C67CC(C6)C7)on5)cccc43)cc2n1] | 413.2 | \ |
| 91s | 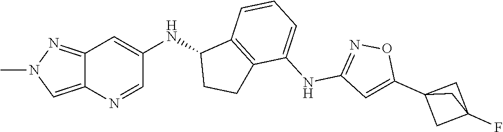 [SMILES: Cn1cc2ncc(N[C@H]3CCc4c(Nc5cc(C67CC(F)(C6)C7)on5)cccc43)cc2n1] | 431.2 | \ |

### Example 92

### (S)—N¹-(2-methyl-2H-pyrazolo[4,3-b]pyridin-6-yl)-N⁴-(5-(3-methyloxetan-3-7)-1,2,4-oxadiazol-3-yl)-2,3-dihydro-1H-indene-1,4-d

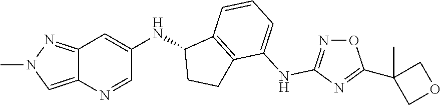 [SMILES: Cn1cc2ncc(N[C@H]3CCc4c(Nc5noc(C6(C)COC6)n5)cccc43)cc2n1]

Compound 92 was prepared by the following steps:

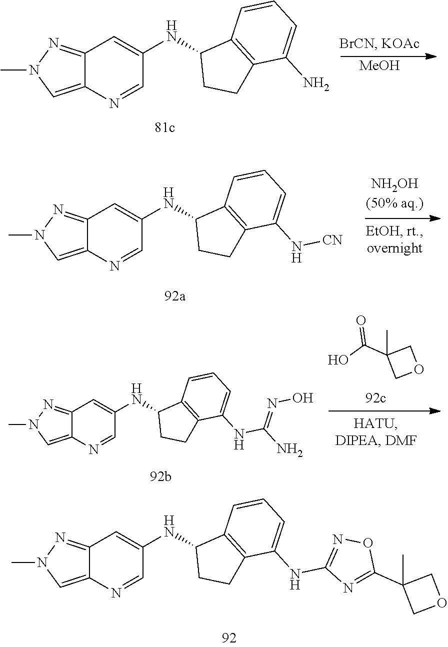

Step 1: To a stirred solution of compound 81c (140 mg, 0.5 mmol) in MeOH (5 mL) cooled to 0˜ 5° C. was added a solution of BrCN (133 mg, 1.25 mmol) in MeOH (5 mL). The resulting mixture was stirred at that temperature for 4-5 h, then concentrated. The residue was dissolved in water (10 mL) and extracted with EtOAc (10 mL*3). The combined organic phases were washed with brine (10 mL), dried over anhydrous Na₂SO₄ and filtered. After concentration, the residue was purified on an ISCO chromatography to afford compound 92a (90 mg, yield: 59.0%) as a solid. ESI-MS (m/z): 305.1 [M+H]⁺.

Step 2: To a solution of compound 92a (152 mg, 0.5 mmol) in EtOH (5 mL) was added 50% aqueous hydroxylamine (83 mg, 1.25 mmol). The resulting mixture was stirred at room temperature overnight, then concentrated to give compound 92b (167.5 mg, 99.3%) as a yellow solid. ESI-MS (m/z): 338.2 [M+H]⁺.

Step 3: To a solution of compound 92c (30 mg, 0.26 mmol) in dry DMF (2 mL) was added DIPEA (33.6 mg, 0.26 mmol) and HATU (99 mg, 0.26 mmol) at room temperature, followed by addition of compound 92b (67 mg, 0.2 mmol) after stirring for 10 min. The resulting mixture was heated to 100˜105° C. and stirred for 1 h. After cooling to room temperature, water (20 mL) was added and the mixture was extracted with EtOAc (20 mL*3). The combined organic phases were dried over anhydrous Na₂SO₄, filtered, and concentrated. The residue was purified on an ISCO chromatography to afford compound 92 (41 mg, yield: 49.1%). ESI-MS (m/z): 418.1 [M+H]⁺. ¹H NMR (62 MHz, Acetone-d₆) δ 8.35-7.69 (m, 4H), 7.48-6.86 (m, 3H), 5.72-5.11 (m, 2H), 5.00 (d, J=6.0 Hz, 2H), 4.71-4.48 (m, 2H), 4.12 (s, 3H), 3.34-2.96 (m, 2H), 2.73-2.18 (m, 1H), 1.82 (s, 3H).

### Example 93

The preparation of compounds 93a-93r refer to the synthesis of compound 92 in example 92.

TABLE 24

| | | MS | |
|---|---|---|---|
| Cpds | Structure | [M + 1]⁺ | ¹H NMR |
| 93a | 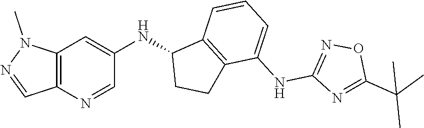 [SMILES: Cn1ncc2ncc(N[C@H]3CCc4c(Nc5noc(C(C)(C)C)n5)cccc43)cc21] | 404.2 | ¹H NMR (400 MHz, DMSO-d₆) δ 9.15 (s, 1H), 8.11 (s, 1H), 7.92 (s, 1H), 7.64 (d, J = 8.0 Hz, 1H), 7.20 (t, J = 7.8 Hz, 1H), 7.10 (s, 1H), 6.98 (d, J = 7.5 Hz, 1H), 6.63 (d, J = 8.3 Hz, 1H), 5.16-5.02 (m, 1H), 3.91 (s, 3H), 3.13-3.02 (m, 1H), 2.93-2.79 (m, 1H), 2.70-2.56 (m, 1H), 1.88-1.73 (m, 1H), 1.39 (s, 9H). |
| 93b | 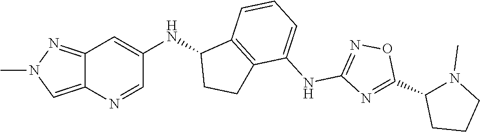 [SMILES: CN1CCC[C@@H]1c1nc(Nc2cccc3c2CC[C@@H]3Nc2cnc3cn(C)nc3c2)no1] | 431.2 | ¹H NMR (400 MHz, DMSO-d₆) δ 9.18 (s, 1H), 8.31 (s, 1H), 8.16 (s, 1H), 7.70-7.59 (m, 1H), 7.25-7.16 (m, 1H), 7.05-6.96 (m, 1H), 6.86 (s, 1H), 6.41-6.33 (m, 1H), 5.07-4.95 (m, 1H), 4.05 (s, 3H), 3.70- 3.62 (m, 1H), 3.09-2.99 (m, 2H), 2.92- 2.80 (m, 1H), 2.62-2.54 (m, 1H), 2.48- 2.32 (m, 4H), 2.30-2.20 (m, 1H), 2.12- 2.01 (m, 1H), 1.94-1.79 (m, 3H). |
| 93c | 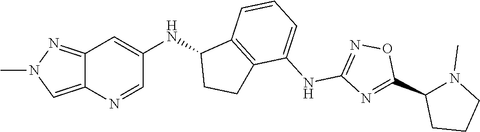 [SMILES: CN1CCC[C@H]1c1nc(Nc2cccc3c2CC[C@@H]3Nc2cnc3cn(C)nc3c2)no1] | 431.2 | ¹H NMR (400 MHz, DMSO-d₆) δ 9.18 (s, 1H), 8.31 (s, 1H), 8.16 (s, 1H), 7.70-7.59 (m, 1H), 7.27-7.15 (m, 1H), 7.06-6.96 (m, 1H), 6.86 (s, 1H), 6.44-6.31 (m, 1H), 5.07-4.95 (m, 1H), 4.05 (s, 3H), 3.71- 3.62 (m, 1H), 3.10-2.99 (m, 2H), 2.92- 2.81 (m, 1H), 2.62-2.55 (m, 1H), 2.47- 2.38 (m, 1H), 2.33 (s, 3H), 2.30-2.20 (m, |
| | | | 1H), 2.11-2.01 (m, 1H), 1.93-1.78 (m, |
| | | | 3H). |
| 93d | 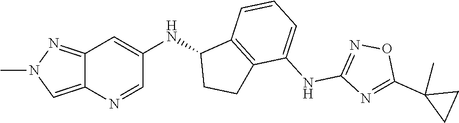 [SMILES: Cn1cc2ncc(N[C@H]3CCc4c(Nc5noc(C6(C)CC6)n5)cccc43)cc2n1] | 402.2 | ¹H NMR (400 MHz, DMSO-d₆) δ 9.06 (s, 1H), 8.30 (s, 1H), 8.14 (d, J = 2.3 Hz, 1H), 7.61 (d, J = 8.1 Hz, 1H), 7.18 (t, J = 7.8 Hz, 1H), 6.98 (d, J = 7.5 Hz, 1H), 6.85 (s, 1H), 6.35 (d, J = 8.1 Hz, 1H), 5.00 (q, J = 7.4 Hz, 1H), 4.04 (s, 3H), 3.10-2.97 (m, 1H), 2.92-2.78 (m, 1H), 2.60-2.55 (m, 1H), 1.88-1.75 (m, 1H), 1.50 (s, 3H), 1.37- |
| | | | 1.28 (m, 2H), 1.13-1.05 (m, 2H). |
| 93e | 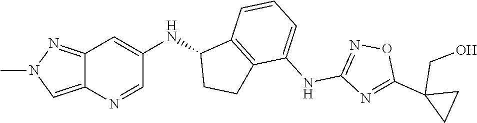 [SMILES: Cn1cc2ncc(N[C@H]3CCc4c(Nc5noc(C6(CO)CC6)n5)cccc43)cc2n1] | 418.2 | ¹H NMR (62 MHz, Acetone-d₆) δ 8.31- 8.07 (m, 2H), 8.01-7.65 (m, 2H), 7.42- 6.84 (m, 3H), 5.49 (s, 1H), 5.32-4.85 (m, 1H), 4.11 (s, 3H), 3.93 (s, 2H), 3.33-2.92 (m, 2H), 2.75-2.35 (m, 1H), 1.97-1.76 (m, 1H), 1.43-1.11 (m, 4H). |
| 93f | 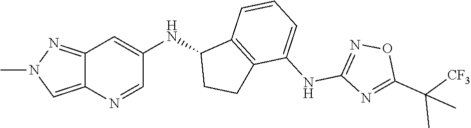 [SMILES: Cn1cc2ncc(N[C@H]3CCc4c(Nc5noc(C(C)(C)C(F)(F)F)n5)cccc43)cc2n1] | 458.2 | ¹H NMR (62 MHz, Acetone-d₆) δ 8.33 8.07 (m, 3H), 7.92-7.68 (m, 1H), 7.44- 6.88 (m, 3H), 5.51 (s, 1H), 5.33-4.92 (m, 1H), 4.11 (s, 3H), 3.33-2.94 (m, 2H), 2.73- 2.38 (m, 1H), 1.97-1.79 (m, 1H), 1.70 (s, 6H). |
| 93g | 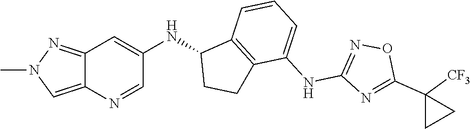 [SMILES: Cn1cc2ncc(N[C@H]3CCc4c(Nc5noc(C6(C(F)(F)F)CC6)n5)cccc43)cc2n1] | 456.2 | ¹H NMR (400 MHz, DMSO-d₆) δ 9.33 (s, 1H), 8.31 (s, 1H), 8.15 (s, 1H), 7.65-7.54 (m, 1H), 7.27-7.16 (m, 1H), 7.08-6.98 (m, 1H), 6.86 (s, 1H), 6.44-6.32 (m, 1H), 5.08-4.96 (m, 1H), 4.04 (s, 3H), 3.12 2.98 (m, 1H), 2.93-2.79 (m, 1H), 2.61- 2.54 (m, 1H), 1.88-1.79 (m, 1H), 1.77- 1.72 (m, 4H). |
| 93h | 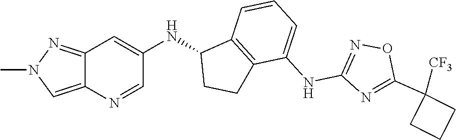 [SMILES: Cn1cc2ncc(N[C@H]3CCc4c(Nc5noc(C6(C(F)(F)F)CCC6)n5)cccc43)cc2n1] | 470.2 | ¹H NMR (400 MHz, DMSO-d₆) δ 9.42 (s, 1H), 8.31 (s, 1H), 8.19-8.13 (m, 1H), 7.68- 7.59 (m, 1H), 7.30-7.13 (m, 1H), 7.08- 6.99 (m, 1H), 6.86 (s, 1H), 6.45-6.33 (m, 1H), 5.10-4.96 (m, 1H), 4.05 (s, 3H), 3.13- 3.01 (m, 1H), 2.94-2.81 (m, 1H), 2.81- 2.69 (m, 4H), 2.62-2.54 (m, 1H), 2.20- 2.07 (m, 2H), 1.89-1.77 (m, 1H). |
| 93i | 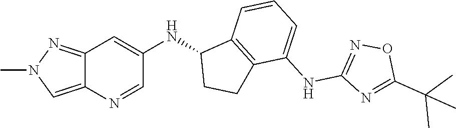 [SMILES: Cn1cc2ncc(N[C@H]3CCc4c(Nc5noc(C(C)(C)C)n5)cccc43)cc2n1] | 404.2 | ¹H NMR (62 MHz, Acetone-d₆) δ 8.33 8.05 (m, 2H), 8.04-7.70 (m, 2H), 7.42- 6.87 (m, 3H), 5.50 (s, 1H), 5.33-4.88 (m, 1H), 4.11 (s, 3H), 3.33-2.94 (m, 2H), 2.74- 2.21 (m, 1H), 1.97-1.66 (m, 1H), 1.42 (s, 9H). |
| 93j | 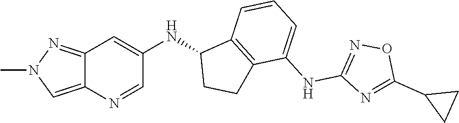 [SMILES: Cn1cc2ncc(N[C@H]3CCc4c(Nc5noc(C6CC6)n5)cccc43)cc2n1] | 388.2 | ¹H NMR (62 MHz, Acetone-d₆) δ 8.30- 8.07 (m, 2H), 7.96-7.66 (m, 2H), 7.42- 6.87 (m, 3H), 5.56-5.36 (m, 1H), 5.34- 4.88 (m, 1H), 4.11 (s, 3H), 3.29-2.95 (m, 2H), 2.75-2.53 (m, 1H), 2.47-2.16 (m, 1H), 1.96-1.79 (m, 1H), 1.36-0.93 (m, 4H). |
| 93k | 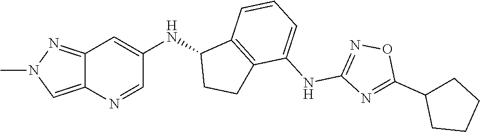 [SMILES: Cn1cc2ncc(N[C@H]3CCc4c(Nc5noc(C6CCCC6)n5)cccc43)cc2n1] | 416.2 | ¹H NMR (400 MHz, DMSO-d₆) δ 9.10 (s, 1H), 8.31 (s, 1H), 8.15 (s, 1H), 7.70-7.59 (m, 1H), 7.26-7.13 (m, 1H), 7.05-6.93 (m, 1H), 6.86 (s, 1H), 6.43-6.28 (m, 1H), 5.07-4.95 (m, 1H), 4.04 (s, 3H), 3.31- 3.22 (m, 1H), 3.13-2.95 (m, 1H), 2.93- 2.80 (m, 1H), 2.65-2.51 (m, 1H), 2.16- 2.03 (m, 2H), 1.92-1.79 (m, 3H), 1.78- |
| | | | 1.61 (m, 4H). |
| 93l | 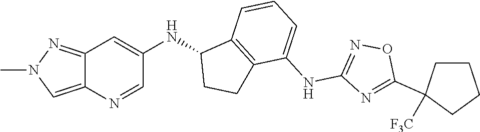 [SMILES: Cn1cc2ncc(N[C@H]3CCc4c(Nc5noc(C6(C(F)(F)F)CCCC6)n5)cccc43)cc2n1] | 484.2 | ¹H NMR (400 MHz, DMSO-d₆) δ 9.39 (s, 1H), 8.31 (s, 1H), 8.15 (s, 1H), 7.67-7.57 (m, 1H), 7.27-7.16 (m, 1H), 7.09-6.98 (m, 1H), 6.86 (s, 1H), 6.43-6.31 (m, 1H), 5.08-4.96 (m, 1H), 4.05 (s, 3H), 3.16- 2.97 (m, 1H), 2.96-2.81 (m, 1H), 2.64- 2.53 (m, 1H), 2.46 (s, 2H), 2.31-2.18 (m, 2H), 1.88-1.69 (m, 5H). |
| 93m | 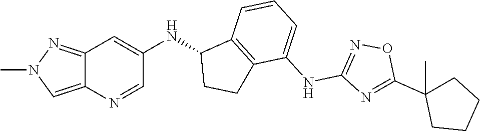 [SMILES: Cn1cc2ncc(N[C@H]3CCc4c(Nc5noc(C6(C)CCCC6)n5)cccc43)cc2n1] | 430.2 | ¹H NMR (400 MHz, DMSO-d₆) δ 9.13 (s, 1H), 8.30 (s, 1H), 8.15 (d, J = 2.2 Hz, 1H), 7.65 (d, J = 8.1 Hz, 1H), 7.19 (t, J = 7.8 Hz, 1H), 6.98 (d, J = 7.5 Hz, 1H), 6.85 (s, 1H), 6.36 (d, J = 8.0 Hz, 1H), 5.00 (q, J = 7.4 Hz, 1H), 4.04 (s, 3H), 3.04 (s, 1H), 2.86 (dt, J = 16.0, 7.8 Hz, 1H), 2.16 (s, 2H), 1.85- 1.69 (m, 8H), 1.42 (s, 3H). |
| 93n | 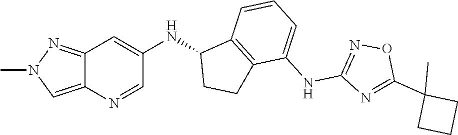 [SMILES: Cn1cc2ncc(N[C@H]3CCc4c(Nc5noc(C6(C)CCC6)n5)cccc43)cc2n1] | 416.2 | ¹H NMR (400 MHz, DMSO-d₆) δ 9.14 (s, 1H), 8.30 (s, 1H), 8.15 (d, J = 2.4 Hz, 1H), 7.65 (d, J = 8.0 Hz, 1H), 7.19 (t, J = 7.8 Hz, 1H), 6.99 (d, J = 7.5 Hz, 1H), 6.85 (s, 1H), 6.36 (d, J = 8.1 Hz, 1H), 5.05-4.96 (m, 1H), 4.04 (s, 3H), 3.11-3.00 (m, 1H), 2.92- 2.81 (m, 1H), 2.59-2.54 (m, 3H), 2.16- 2.04 (m, 3H), 2.03-1.93 (m, 1H), 1.87- 1.76 (m, 1H), 1.59 (s, 3H). |
| 93o | 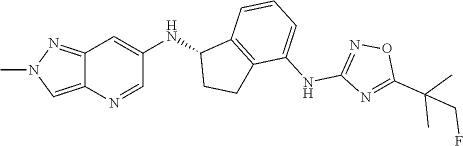 [SMILES: Cn1cc2ncc(N[C@H]3CCc4c(Nc5noc(C(C)(C)CF)n5)cccc43)cc2n1] | 422.2 | ¹H NMR (400 MHz, DMSO-d₆) δ 9.21 (s, 1H), 8.31 (s, 1H), 8.15 (s, 1H), 7.63 (d, J = 8.0 Hz, 1H), 7.20 (t, J = 7.8 Hz, 1H), 7.00 (d, J = 7.5 Hz, 1H), 6.86 (s, 1H), 6.37 (d, J = 8.3 Hz, 1H), 5.01 (q, J = 6.4 Hz, 1H), 4.66 (s, 1H), 4.54 (s, 1H), 4.04 (s, 3H), 3.10-3.00 (m, 1H), 2.92-2.81 (m, 1H), 2.60-2.54 (m, 1H), 1.88-1.76 (m, 1H), 1.41 (s, 6H). |
| 93p | 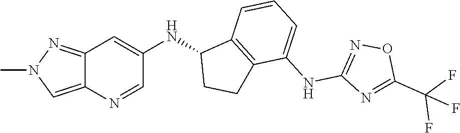 [SMILES: Cn1cc2ncc(N[C@H]3CCc4c(Nc5noc(C(F)(F)F)n5)cccc43)cc2n1] | 416.1 | ¹H NMR (400 MHz, DMSO-d₆) δ 9.78 (s, 1H), 8.31 (s, 1H), 8.15 (d, J = 2.3 Hz, 1H), 7.57 (d, J = 8.0 Hz, 1H), 7.25 (t, J = 7.7 Hz, 1H), 7.08 (d, J = 7.4 Hz, 1H), 6.87 (d, J = 2.3 Hz, 1H), 6.39 (d, J = 8.1 Hz, 1H), 5.11- 4.96 (m, 1H), 4.04 (s, 3H), 3.12-2.98 (m, 1H), 2.95-2.80 (m, 1H), 2.63-2.56 (m, 1H), 1.90-1.77 (m, 1H). |
| 93q | 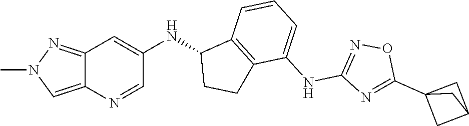 [SMILES: Cn1cc2ncc(N[C@H]3CCc4c(Nc5noc(C67CC(C6)C7)n5)cccc43)cc2n1] | 414.2 | ¹H NMR (400 MHz, DMSO-d₆) δ 9.17 (s, 1H), 8.31 (s, 1H), 8.15 (s, 1H), 7.66-7.59 (m, 1H), 7.23-7.16 (m, 1H), 7.03-6.97 (m, 1H), 6.86 (s, 1H), 6.41-6.32 (m, 1H), 5.07-4.95 (m, 1H), 4.04 (s, 3H), 3.10- 2.97 (m, 1H), 2.91-2.79 (m, 1H), 2.61 (s, 1H), 2.59-2.53 (m, 1H), 2.28 (s, 6H), 1.89- 1.75 (m, 1H). |
| 93r | 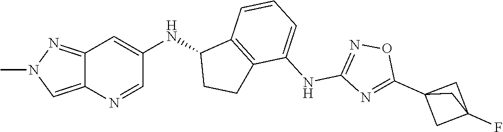 [SMILES: Cn1cc2ncc(N[C@H]3CCc4c(Nc5noc(C67CC(F)(C6)C7)n5)cccc43)cc2n1] | 432.2 | ¹H NMR (400 MHz, DMSO-d₆) δ 9.25 (s, 1H), 8.31 (s, 1H), 8.15 (s, 1H), 7.67-7.57 (m, 1H), 7.25-7.17 (m, 1H), 7.06-6.97 (m, 1H), 6.86 (s, 1H), 6.45-6.32 (m, 1H), 5.07-4.95 (m, 1H), 4.04 (s, 3H), 3.11- 2.97 (m, 1H), 2.91-2.77 (m, 1H), 2.62 (s, 6H), 2.59-2.52 (m, 1H), 1.89-1.75 (m, 1H). |

### Example 94

### (S)—N⁴-(5-(tert-butyl) isoxazol-3-yl)-N¹-methyl-N¹-(1-methyl-1H-pyrazolo[4,3-b]pyridin-6-yl)-2,3-dihydro-1H-indene-1,4-diamine

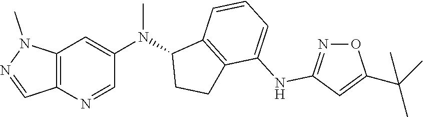 [SMILES: CN(c1cnc2cnn(C)c2c1)[C@H]1CCc2c(Nc3cc(C(C)(C)C)on3)cccc21]

Compound 94 was prepared by the following steps:

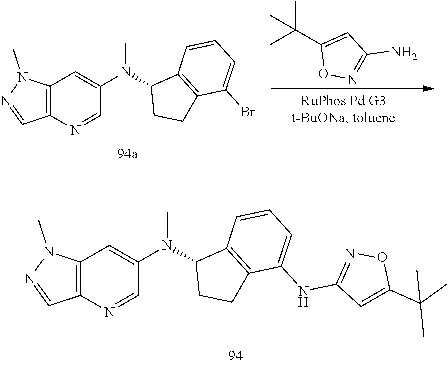

The preparation of compound 94 refer to the synthesis of compound 41 in example 41, wherein the preparation of compound 94a refer to the synthesis of compound 82b in example 82.

ESI-MS (m/z): 417.2 [M+H]⁺. ¹H NMR (62 MHz, acetone-d₆) δ 8.66-8.44 (m, 1H), 8.06-7.81 (m, 2H), 7.72-7.51 (m, 1H), 7.39-7.04 (m, 2H), 6.90-6.66 (m, 1H), 6.00-5.87 (m, 1H), 5.85-5.58 (m, 1H), 3.98 (s, 3H), 3.20-2.81 (m, 2H), 2.76 (s, 3H), 2.57-1.87 (m, 2H), 1.33 (s, 9H).

### Example 95

### (S)-5-(tert-butyl)-N-(1-((1-methyl-1H-pyrazolo[4,3-b]pyridin-6-yl)oxy)-2,3-dihydro-1H-inden-4-yl) isoxazol-3-amine

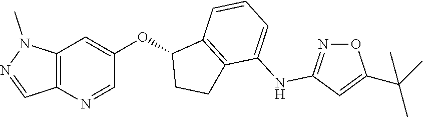 [SMILES: Cn1ncc2ncc(O[C@H]3CCc4c(Nc5cc(C(C)(C)C)on5)cccc43)cc21]

Compound 95 was prepared by the following steps:

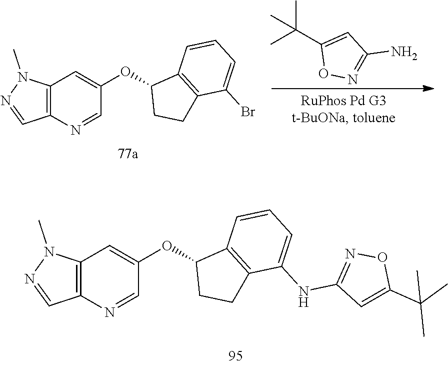

The preparation of compound 95 refer to the synthesis of compound 41 in example 41.

ESI-MS (m/z): 404.2 [M+H]⁺. ¹H NMR (62 MHz,) δ 8.73 (d, J=2.5 Hz, 1H), 8.67-8.43 (m, 2H), 8.27-8.04 (m, 2H), 7.94-7.74 (m, 1H), 7.73-7.46 (m, 1H), 6.63-6.50 (m, 1H), 6.45 (s, 1H), 4.57 (s, 3H), 3.73-3.41 (m, 2H), 3.27-3.07 (m, 1H), 2.98-2.69 (m, 1H), 1.83 (s, 9H).

### Example 96

The preparation of compounds 96a-96d refer to the synthesis of compound 58 in example 58.

TABLE 25

| | | MS | |
|---|---|---|---|
| Cpds | Structure | [M + 1]⁺ | ¹HNMR |
| 96a | 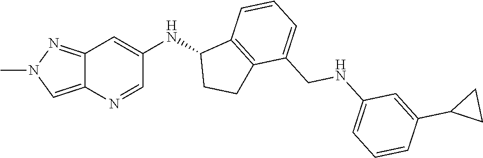 [SMILES: Cn1cc2ncc(N[C@H]3CCc4c(CNc5cccc(C6CC6)c5)cccc43)cc2n1] | 410.2 | ¹H NMR (62 MHz, DMSO-d₆) δ 8.29 (s, 1H), 8.15 (d, J = 2.6 Hz, 1H), 7.18 (s, 3H), 7.02- 6.69 (m, 2H), 6.46-6.20 (m, 3H), 6.20-5.86 (m, 2H), 5.17-4.78 (m, 1H), 4.39-4.14 (m, 2H), 4.03 (s, 3H), 3.13-2.75 (m, 2H), 2.03- 1.55 (m, 2H), 1.36-1.11 (m, 1H), 0.99-0.75 (m, 2H), 0.73-0.48 (m, 2H). |
| 96b | 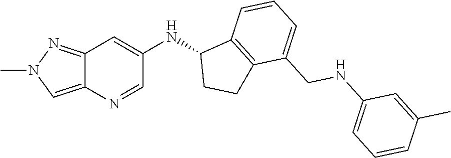 | 384.2 | ¹H NMR (62 MHz, DMSO-d₆) δ 8.78-8.52 (m, 2H), 7.86-7.57 (m, 3H), 7.51-7.26 (m, 2H), 7.08-6.75 (m, 3H), 6.12-5.70 (m, 2H), 5.69-5.29 (m, 2H), 4.89-4.66 (m, 2H), 4.56 (s, 3H), 3.70-3.35 (m, 2H), 3.11-2.89 (m, 1H), 2.65 (s, 3H). |
| 96c | 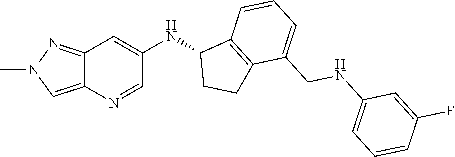 [SMILES: Cn1cc2ncc(N[C@H]3CCc4c(CNc5cccc(F)c5)cccc43)cc2n1] | 388.2 | ¹H NMR (62 MHz, DMSO-d₆) δ 8.67 (d, J = 2.5 Hz, 1H), 8.59 (s, 1H), 7.90-7.55 (m, 4H), 7.55-7.33 (m, 2H), 7.12-6.84 (m, 2H), 6.83-6.56 (m, 1H), 6.30-5.86 (m, 2H), 5.75-5.33 (m, 1H), 4.91-4.72 (m, 2H), 4.56 (s, 3H), 3.69-3.34 (m, 2H), 3.23-2.91 (m, 1H). |
| 96d | 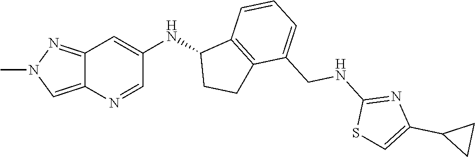 [SMILES: Cn1cc2ncc(N[C@H]3CCc4c(CNc5nc(C6CC6)cs5)cccc43)cc2n1] | 417.2 | ¹H NMR (62 MHz, DMSO-d₆) δ 8.67 (d, J = 2.5 Hz, 1H), 8.58 (s, 1H), 7.88-7.60 (m, 3H), 7.57-7.27 (m, 2H), 6.57 (s, 1H), 6.10-5.79 (m, 1H), 5.74-5.32 (m, 1H), 5.07-4.83 (m, 2H), 4.55 (s, 3H), 3.69-3.35 (m, 2H), 3.32- 3.04 (m, 2H), 2.28-2.00 (m, 1H), 1.35-1.23 (m, 2H), 1.19-1.06 (m, 2H). |

### Example 97

### (S)-1-((1H-pyrazolo[4,3-b]pyridin-6-yl)amino)-N-(3-cyclopropylphenyl)-2,3-dihydro-1H-indene-4-carboxamide

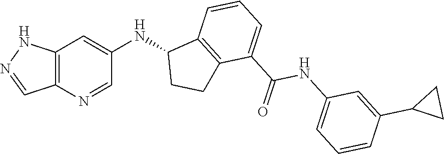 [SMILES: O=C(Nc1cccc(C2CC2)c1)c1cccc2c1CC[C@@H]2Nc1cnc2cn[nH]c2c1]

Compound 97 was prepared by the following steps:

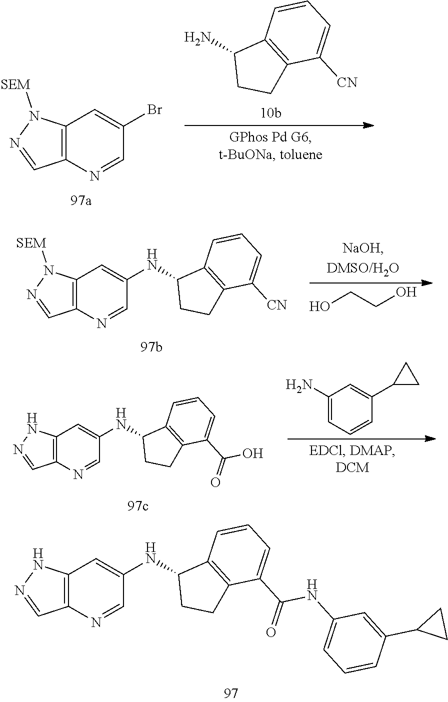

The preparation of compound 97 refer to the synthesis of compound 73 in example 73.

ESI-MS (m/z): 410.2 [M+H]⁺. ¹H NMR (62 MHz, acetone-d₆) δ 11.83 (s, 1H), 9.34 (s, 1H), 8.46-6.68 (m, 10H), 6.01-5.57 (m, 1H), 5.41-4.81 (m, 1H), 4.06-3.17 (m, 2H), 2.97-2.32 (m, 1H), 1.88-1.52 (m, 1H), 1.41-0.41 (m, 5H).

### In Vitro Inhibitory Activity of Kinases

CSF-1R, PDGFR-α, KDR (VEGFR2) and KIT in vitro Kinase Activity Assay:

### 1. Materials and Instruments

ENVISION (Perkinelmer, 2104)
White 384-well MicroPlate (Cat #264706, Nunc)
HTRF kinEASE TK kit main reagents (Cat #62TKOPEC, Cisbio)
TK-biotin substrate
Streptavidin-XL665
TK Antibody-Cryptate
5× Enzymatic buffer (5×KB)
Staphylococcal enterotoxin B (SEB)
HTRF Detection buffer
CSF-1R (Cat #PV3249, Invitrogen)
PDGFR-α (Cat #PV3811, Invitrogen)
KDR (Cat #PV3660, Invitrogen)
KIT (Cat #08-156, Carna)
ATP 10 mM (Cat #PV3227, Invitrogen)
DTT 1 M (Cat #D5545, Sigma)
MgCl₂ 1 M (Cat #M8266, Sigma)
MnCl₂ 1 M (Cat #244589, Sigma)

### 2. Experimental Procedure

### 2.1 Reagent Preparation

TABLE 26
Composition and Concentration Table of Each Component in the Kinase Reaction
System

| | Final Concentration in the | | | | | | | | |
|---|---|---|---|---|---|---|---|---|---|
| | Kinase Reaction Step (10 μL) | | | | | | | Final Concentration in the Total | |
| | | | | | | | Enzyme | Reaction (20 μL) | |
| | Enzyme | | | | TK- | | Reaction | Streptavidin- | TK Antibody- |
| | Concentration | | ATP | | substrate | | Time | XL665 | Cryptate |
| CSF-1R | 10 | ng/well | 5 | μM | 1 | μM | 60 min | 125 nM | 1:100 dilution |
| PDGFR-α | 0.8 | ng/well | 5 | μM | 1 | μM | 40 min | 125 nM | 1:100 dilution |
| KDR | 0.1 | ng/well | 0.2 | μM | 0.2 | μM | 30 min | 25 nM | 1:100 dilution |
| KIT | 2 | ng/well | 20 | μM | 1 | μM | 30 min | 125 nM | 1:100 dilution |

TABLE 27
Components of 1 mL 1 × Kinase Buffer (uL)

| Kinase | 5 × Enzyme buffer | MgCl₂ | MnCl₂ | DTT | SEB | ddH₂O |
|---|---|---|---|---|---|---|
| CSF-IR | 200 | 5 | 1 | 1 | 20 | 773 |
| PDGFR-α | 200 | 5 | 1 | 1 | 8 | 785 |
| KDR | 200 ul | 5 | 1 | 1 | 10 | 783 |
| KIT | 200 ul | 5 | 1 | 0 | 10 | 784 |

### 5×Substrate-TK and ATP Working Solution

The specific concentrations of Substrate-TK and ATP are shown in Table 26.

Dilute Substrate-TK and ATP to 5× of the reaction concentration using 1× Kinase Buffer.

### 5× Enzyme Working Solution

The concentrations of the four kinases are shown in Table 26. Prepare the 5× enzyme working solution using 1× Kinase Buffer.

### 4×Sa-XL665 Working Solution

The concentration of Sa-XL665 in the reaction is shown in Table 26. Prepare the 4×Sa-XL665 working solution using Detection Buffer.

### 4×TK-Ab-Cryptate Working Solution

Dilute TK-Ab-Cryptate 100 times using Detection Buffer to prepare the working solution.

### 2.2 Experimental Procedure

After all reagents were prepared according to the methods described above, except for the enzyme, they were equilibrated to room temperature before starting the sample addition.
a) First, 2.5% DMSO solutions were prepared separately using the pre-prepared 1× kinase buffer (DMSO concentrations that were too high could affect the reaction, so the final concentration of DMSO was controlled at 1%). Then, the corresponding 2.5% DMSO solution for the enzyme was used to dilute the test compounds. Compounds were screened with two concentrations of 100 nM and 10 nM. Except for the control wells, 4 μL of the diluted test compound solution was added to the reaction wells, and 4 μL of the pre-prepared 2.5% DMSO solution was added to the control wells.
b) 2 μL of the pre-prepared TK-biotin substrate solution at the corresponding concentration for the kinase was added to all reaction wells.
c) 2 μL of the pre-prepared enzyme solution at the corresponding concentration was added to all reaction wells except the negative control wells. For the negative control wells, 2 μL of 1× kinase buffer corresponding to the enzyme was added to make up the volume. The plate was sealed with sealing film, mixed gently, and incubated at room temperature for 10 min to allow the compounds to fully interact with the enzyme.
d) 2 μL of the ATP solution at the corresponding concentration for the enzyme was added to all reaction wells to initiate the kinase reaction. ATP concentrations and reaction times for enzyme screening are shown in Table 26
e) Five minutes before the end of the kinase reaction, the detection solution was prepared. The detection buffer from the kit was used to prepare detection solutions for Streptavidin-XL665 and TK antibody europium cryptate (1:100) at the appropriate concentrations for the two enzymes.
f) After the kinase reaction was completed, 5 μL of the diluted Streptavidin-XL665 solution was added to all reaction wells. After mixing, the diluted TK antibody europium cryptate detection solution was immediately added.
g) The plate was sealed and mixed, incubated at room temperature for 1 hour, and the fluorescence signal was detected using the ENVISION (PerkinElmer) instrument (excitation at 320 nm, emission at 665 nm and 615 nm). The inhibition rate for each well was calculated based on the full-activity control wells and background signal wells, and the average value of the replicates was taken.
   Experimental Procedure as follows:

TABLE 28

| | Kinase | | |
|---|---|---|---|
| Enzyme Step | Assay | Control | |
| (10 μL) | Sample | Negative | Positive |
| Compound | 4 μL | 4 μL 2.5% DMSO/kinase | 4 μL 2.5% DMSO/kinase |
| | | buffer | buffer |
| TK Substrate-biotin | 2 μL | 2 μL | 2 μL |
| Kinase | 2 μL | 2 μL Kinase buffer | 2 μL |

Seal the plate, incubate at room temperature for 10 min

| | | | |
|---|---|---|---|
| ATP | 2 μL | 2 μL | 2 μL |

Seal the plate, the incubation times of the kinases
at room temperature are shown in Table 26
Detection Step (10 μL)

| | | | |
|---|---|---|---|
| Sa-XL665 | 5 μL | 5 μL | 5 μL |
| TK Ab-Cryptate | 5 μL | 5 μL | 5 μL |

Seal the plate, incubate at room temperature for 1 hour
Excitation at 320 nm, emission at 665 nm and 615 nm

### 2.3 Data Analysis

$$Emission Ratio ( E R ) = 665 nm Emission signal / 615 nm Emission signal$$

$$Inhibition rate = ( E {R}_{positive} - E {R}_{sample} ) / ( E {R}_{positive} - E {R}_{negative} ) * 100 \%$$

DDR1 in vitro Kinase Activity Assay:

### 1. Materials and Instruments

ENVISION (Perkinelmer, 2104)
White 384-well MicroPlate (Cat #264706, Nunc)
DDR1 (Cat #PV6047, Invitrogen)
5× Kinase Buffer A (Cat #PV3189, Invitrogen)
Kinase Tracer 178 (Cat #PV5593, Invitrogen)
LanthaScreen® Eu-anti-GST Antibody (Cat #PV5594, Invitrogen)

### 2. Experimental Procedure

TABLE 29
Composition and Concentration Table of Each
Component in the Kinase Reaction System

| | Final Concentration in the Kinase Reaction Step (15 μL) | | | |
|---|---|---|---|---|
| | | LanthaScreen ® | | Enzyme |
| | Enzyme | Eu-anti-GST | Kinase | Reaction |
| | Concentration | Antibody | Tracer 178 | Time |
| DDR1 | 5 nM | 2 nM | 10 nM | 60 min |

### 2.1 Reagent Preparation

The compounds to be tested were tested at two concentrations of 100 nM and 10 nM.

Kinase/Antibody Mixtures Preparation: Dilute Kinase/Antibody Mixtures with 1× Kinase Buffer to form 3× working solutions with final concentrations of 5 nM kinase and 2 nM Eu-Anti-GST Antibody, respectively.

Kinase Tracer 178 Preparation: Dilute Kinase Tracer 178 with 1× Kinase Buffer to make a 3× working solution with a final concentration of 10 nM.

### 2.2 Experimental Procedure

After all reagents were prepared according to the methods described above, except for the enzyme, they were equilibrated to room temperature before starting the sample addition.
a) First, 3% DMSO solutions were prepared separately using the pre-prepared 1× kinase buffer (DMSO concentrations that were too high could affect the reaction, so the final concentration of DMSO was controlled at 1%). Then, the corresponding 3% DMSO solution for the enzyme was used to dilute the test compounds. Compounds were screened with two concentrations of 100 nM and 10 nM. Except for the control wells, 5 μL of the diluted test compound solution was added to the reaction wells, and 5 μL of the pre-prepared 3% DMSO solution was added to the control wells.
b) 5 μL of the pre-prepared Kinase/Antibody mixtures was added to all reaction wells except the negative control wells. For the negative control wells, 5 μL of 1× kinase buffer corresponding to the enzyme was added to make up the volume.
c) 5 μL of the pre-prepared Kinase Tracer 178 solution was added to all reaction wells.
d) The plate was sealed and mixed, incubated at room temperature for 1 hour, and the fluorescence signal was detected using the ENVISION (PerkinElmer) instrument (excitation at 340 nm, emission at 665 nm and 615 nm). The inhibition rate for each well was calculated based on the full-activity control wells and background signal wells, and the average value of the replicates was taken.

Experimental Procedure as follows:

TABLE 30

| | Kinase | | |
|---|---|---|---|
| Enzyme Step | Assay | Control | |
| (10 μL) | Sample | Negative | Positive |
| Compounds | 5 μL | 5 μL 3% | 5 μL 3% |
| | | DMSO/kinase | DMSO/kinase |
| | | buffer | buffer |
| Kinase/ | 5 μL | 5 μL Kinase | 5 μL |
| Antibody Mixtures | | buffer | |
| Kinase Tracer 178 | 5 μL | 5 μL | 5 μL |

Seal the plate, incubate at room temperature for 1 h
Excitation at 340 nm, emission at 665 nm and 615 nm

### 2.3 Data Analysis

$$Emission Ratio ( E R ) = 665 nm Emission signal / 615 nm Emission signal$$

$$Inhibition rate = ( E {R}_{positive} - E {R}_{sample} ) / ( E {R}_{positive} - E {R}_{negative} ) * 100 \%$$

TABLE 31

| | CSF-1R | | KIT | | PDGFRα | | VEGFR2 | | DDR1 | |
|---|---|---|---|---|---|---|---|---|---|---|
| Comp. | 100 nM | 10 nM | 100 nM | 10 nM | 100 nM | 10 nM | 100 nM | 10 nM | 100 nM | 10 nM |
| 4 | 89.8 | 80.1 | 99.2 | 76.8 | 96.8 | 90.9 | 100.5 | 97.8 | 98.2 | 88.6 |
| 13 | 93.5 | 79.9 | 97.5 | 73.0 | 99.1 | 78.7 | 99.5 | 95.3 | 95.1 | 90.2 |
| 14aa | 95.3 | 74.6 | 97.3 | 61.9 | 98.7 | 72.7 | 99.6 | 83.1 | 104.5 | 93.2 |
| 14ah | 101.0 | 41.0 | 83.3 | 11.3 | 99.4 | 67.6 | 58.1 | 26.8 | 85.6 | 37.9 |
| 14ap | 96.7 | 63.5 | 80.7 | 30.5 | 98.8 | 72.8 | 66.2 | 14.7 | 100.4 | 75.8 |
| 14as | 97.0 | 89.7 | 94.7 | 58.7 | 101.1 | 91.0 | 101.7 | 88.4 | 103.4 | 94.7 |
| 14ba | 95.9 | 42.1 | 95.0 | 60.1 | 100.3 | 75.5 | 82.6 | 29.2 | 61.4 | 32.4 |
| 15 | 90.5 | 49.0 | 31.6 | 13.3 | 64.0 | 39.6 | 97.3 | 60.0 | 88.0 | 15.9 |
| 19a | 100.3 | 79.4 | 48.6 | 22.1 | 86.2 | 40.5 | 100.7 | 92.3 | 101.5 | 69.4 |
| 19b | 97.4 | 71.9 | 80.2 | 11.2 | 97.1 | 22.9 | 101.5 | 93.6 | 104.7 | 84.0 |
| 19f | 95.4 | 27.5 | 53.9 | 12.1 | 67.2 | 14.5 | 88.9 | 22.7 | 53.5 | 23.1 |
| 19h | 96.6 | 72.0 | 4.9 | −3.9 | 68.3 | 3.8 | 84.4 | 24.6 | 97.1 | 65.3 |
| 19j | 100.7 | 80.1 | 62.8 | 14.5 | 79.7 | 23.5 | 101.7 | 91.7 | 96.7 | 86.1 |
| 19k | 97.4 | 57.6 | 63.7 | 21.8 | 74.3 | 13.9 | 99.1 | 53.0 | 89.9 | 81.4 |
| 21 | 100.3 | 48.6 | 15.3 | 2.4 | 38.7 | 7.7 | 62.6 | 22.3 | 88.0 | 29.7 |
| 22a | 96.7 | 34.0 | 27.2 | 5.8 | 60.5 | 62.9 | 96.4 | 52.5 | 86.0 | 32.4 |
| 27g | 95.8 | 75.2 | 100.5 | 89.6 | 98.9 | 94.7 | 100.0 | 88.9 | 101.4 | 92.1 |
| 41 | 100.2 | 26.9 | 66.4 | 7.5 | 100.2 | 55.5 | 63.9 | 18.6 | 78.2 | 20.2 |
| 420 | 100.3 | 55.8 | 38.8 | 14.5 | 57.8 | 15.4 | 65.6 | 28.9 | 67.6 | 4.1 |
| 73 | 100.9 | 70.2 | 99.7 | 59.1 | 100.0 | 90.0 | 100.1 | 68.3 | 103.1 | 72.5 |
| 74h | 96.3 | 72.6 | 94.9 | 35.3 | 94.8 | 29.3 | 94.9 | 60.3 | 99.9 | 57.2 |
| 76ac | 97.3 | 71.9 | 54.0 | 12.6 | 53.1 | 13.7 | 98.3 | 85.4 | 103.1 | 72.5 |
| 76ai | 99.1 | 78.2 | 79.1 | 39.6 | 89.3 | 37.8 | 101.6 | 96.5 | 99.6 | 94.2 |
| 76al | 103.4 | 104.8 | 95.9 | 91.2 | 97.2 | 97.6 | 95.6 | 99.1 | 98.5 | 98.0 |
| 76aw | 99.1 | 65.1 | 92.4 | 56.0 | 101.9 | 88.5 | 101.0 | 89.9 | 93.6 | 64.0 |
| 76bd | 94.1 | 15.3 | 31.4 | 5.6 | 85.7 | 32.0 | 97.0 | 93.2 | 69.7 | 21.2 |
| 80k | 96.5 | 73.7 | 47.8 | 22.0 | 51.2 | 19.0 | 91.9 | 43.0 | 83.9 | 14.7 |
| 80l | 98.4 | 24.3 | 104.5 | 69.9 | 96.9 | 69.1 | 100.4 | 95.0 | 97.9 | 75.1 |
| 86 | 95.9 | 46.6 | 82.0 | 23.4 | 96.5 | 26.8 | 70.1 | 12.9 | 98.4 | 20.0 |

### M-NFS-60 Cell In Vitro Assay Procedure

### 1. Experimental Purpose

To determine the inhibitory activity of compounds on M-NFS-60 cells.

### 2. Materials

M-NFS-60 cell line (ATCC, Cat. No.: CRL-1838)
RPMI-1640 medium (Gibco, domestic subpackage, Cat. No.: C11875500BT)
Recombinant Mouse CSF1/M-CSF (Sino Biological Inc., Cat. No.: 51112-MNAH)
PAN/Serum (BioTECH, Cat. No.: P30-2602)
Trypsin/0.05% Trypsin-EDTA (Gibco, Cat. No.: 25300-062)
0.4% Trypan Blue (Sigma, Cat. No.: T8154-20 ml)
Penicillin-Streptomycin (Hyclone, Cat. No.: SV30010)
DMSO (Solarbio, Cat. No.: D8371)
PBS (Solarbio, Cat. No.: P1020)
CCK (AbMole BioScience, Cat. No.: M4839-500Tests)

### 3. Instruments

TABLE 32

| Instruments | Manufacturer |
|---|---|
| Biological safety cabinet | ThermoFisherScientific |
| 371 direct-heated CO₂ incubator | ThermoFisherScientific |
| Electronic digital display | Changzhou Guohua |
| constant temperature | Electric Appliance |
| water bath | Co., Ltd. |
| Inverted microscope | OLYMPUS |
| Hemocytometer | Shanghai Qiujing t |
| | Biochemical Reagen |
| | and Instrument Co., Ltd. |
| Shaker | IKA |
| 4° C. refrigerator | ThermoFisherScientific |
| −20° C. refrigerator | Haier |
| L-500 centrifuge | Hunan Xiangyi Centrifuge |
| −80° C. ultra-low | ThermoFisherScientific |
| temperature refrigerator | |
| Liquid nitrogen storage tank | ThermoFisherScientific |
| Full-wavelength reader/ | ThermoFisherScientific |
| microplate reader | |

### 4. Experimental Purpose

### 4.1 Experimental Preparation

1) M-CSF factor stock solution: take 10 μg of powdered m-CSF factor, add 0.4 ml of saline, formulate 25 ug/ml and store at −80° C.
2) M-CSF factor (25 ng/ml) medium:
1640 (Gibco)+10% PAN+100×Penicillin-Streptomycin+100×M-CSF stock solution
3) Complete medium:
1640 (Gibco)+10% PAN+100×Penicillin-Streptomycin
4) Compound preparation: 2-3 mg of the compound was added to DMSO to formulate a 30 mM stock solution, −80° C. refrigerator storage

### 4.2 Experimental Procedure

1) Cell resuscitation: the frozen cells in the cryovial were thawed rapidly by shaking in a 37° C. water bath, then 4 mL of medium was added. After mixing evenly, they were centrifuged at 125×g for 5 min, and the supernatant was discarded. The cells were resuspended and transferred to the culture flasks, and were cultured in a 5% CO₂ fully saturated humidity incubator.
2) Cell passaging: M-NFS-60 cells are semi-suspension cells, which were collected directly in a 15 mL centrifuge tube, centrifuged at 125×g for 5 min. and the supernatant was discarded. The cells were resuspended and passaged into new culture flasks at a ratio of 1:5 to 1:10.
3) After the newly resuscitated cells were passaged 1-2 times for cell stabilization, compound inhibition assays were started when the cells were in the logarithmic growth phase.
(a) Cell collection and counting: the same as the cell passaging step, centrifuge in two tubes, one tube with M-CSF factor medium to resuspend the cells, the other tube with complete medium to resuspend the cells. Take 10 μL of cell suspension in each tube, and count the cells by Tepan blue staining.
(b) Dilute the cell solution according to the concentration of inoculated cells respectively, the concentration of M-NFS-60 cells inoculated with factor was 5.56×10⁴ cells/mL, and the concentration of inoculated without factor was 5×10⁴ cells/mL.
(c) Seed plate: 384 well plate inoculated with M-NFS-60 cells, 36 uL/well, triplicate wells, the number of inoculated cells was equivalent to 2000 cells/well, set with blank control, factor free negative control, normal control, DMSO control and experimental wells. The cells were placed in a 5% CO₂ fully saturated humidity incubator for 4 h.
(d) Sampling compounds: 96 well plates were used for compound preparation. The compound solution prepared in advance was diluted to corresponding gradient concentration with DMSO. An appropriate amount of the diluted solution in the previous step was added to an equal volume of factor containing medium. The 96 well plates were centrifuged at 1000 rpm for 5 min, which played the role of defoaming and spinning down droplets and condensation. Remove the 384 well plate from the incubator, add the diluted compound solution into each well sequentially, 4 μL/well; DMSO wells and normal wells were added with corresponding factor containing medium, 4 μL/well. Then it was put into CO₂ incubator for 48 h.
(e) After incubation for 48 h, CCK was added to each well, 4 uL, and the plates were placed in the incubator for another 2 h, and then the OD (optical density) value at 450 nm was measured by microplate reader.

The experimental flow chart is as follows:

TABLE 33

| | Groups | | | |
|---|---|---|---|---|
| | Blank | | Normal and DMSO | |
| Step | control | Negative control | control | Test |
| Cell | 40 | 40 μL resuspended | 36 μLresuspended | 36 μLresuspended |
| suspension | μLfactor | cell solution | cell solution | cell solution |
| | containing | containing | containing | containing |
| | medium | complete medium | complete medium | complete medium |
| Compound | / | / | 4 μL | 4 μL |
| solution | | | factor containing | Compound solution |
| | | | medium | of corresponding |
| | | | | concentration |
| CCK | 4 μL | 4 μL | 4 μL | 4 μL |

### 4.3 Data Analysis

$$Inhibition rate \% = 1 - ( O D value of the test well - O D value of the blank control ) / ( O D value of the DMSO well - O D value of the blank control ) \times 100 \% .$$

The IC₅₀ values for each test compound were fitted using professional graphing and analysis software.

TABLE 34

| | M-NFS-60cellassay | | M-NFS-60cellassay |
|---|---|---|---|
| Cpds | IC₅₀(nM) | Cpds | IC₅₀(nM) |
| 1f | *** | 1 | *** |
| 2a | * | 2b | * |
| 2c | ** | 3a | * |
| 3b | **** | 3c | *** |
| 3d | **** | 3e | **** |
| 3f | **** | 3g | **** |
| 3h | * | 3i | * |
| 3j | * | 4 | **** |
| 5a | **** | 5b | *** |
| 5c | *** | 5d | **** |
| 5e | **** | 6 | *** |
| 7 | **** | 8d | *** |
| 8 | **** | 9 | *** |
| 10 | *** | 11 | *** |
| 12 | **** | 13 | **** |
| 14aa | **** | 14ab | **** |
| 14ac | **** | 14ad | **** |
| 14ae | **** | 14af | **** |
| 14ag | **** | 14ah | **** |
| 14ai | *** | 14aj | *** |
| 14ak | **** | 14al | **** |
| 14am | **** | 14an | **** |
| 14ao | **** | 14ap | **** |
| 14aq | ** | 14ar | ** |
| 14as | **** | 14at | *** |
| 14au | *** | 14av | *** |
| 14aw | **** | 14ax | **** |
| 14ay | *** | 14az | *** |
| 14ba | **** | 15 | ** |
| 16 | * | 17 | **** |
| 18 | *** | 19a | **** |
| 19b | **** | 19c | *** |
| 19d | *** | 19e | *** |
| 19f | *** | 19g | * |
| 19h | * | 19i | *** |
| 19j | **** | 19k | **** |
| 19l | * | 19m | ** |
| 19n | **** | 19o | *** |
| 19p | **** | 19q | **** |
| 19r | *** | 19s | ** |
| 19t | *** | 19u | * |
| 19v | ** | 19w | ** |
| 20 | **** | 21 | *** |
| 22a | **** | 22b | **** |
| 22c | *** | 22d | *** |
| 22e | ** | 22f | * |
| 22g | **** | 23 | * |
| 24 | **** | 25a | **** |
| 25b | * | 25c | **** |
| 25d | * | 25e | *** |
| 25f | *** | 26 | ***** |
| 27a | **** | 27b | * |
| 27c | **** | 27d | * |
| 27e | *** | 27f | **** |
| 27g | **** | 27h | * |
| 27i | **** | 27j | * |
| 27k | **** | 27l | * |
| 27m | * | 27n | **** |
| 27o | **** | 28a | * |
| 28b | * | 29 | * |
| 30 | **** | 31 | ** |
| 32 | * | 33c | **** |
| 33 | *** | 34 | *** |
| 35 | *** | 36a | * |
| 36b | *** | 36c | **** |
| 37 | *** | 38 | *** |
| 39a | * | 39b | * |
| 40d | *** | 40 | **** |
| 41 | **** | 42a | *** |
| 42b | *** | 42c | **** |
| 42d | * | 42e | * |
| 42f | * | 42g | * |
| 42h | * | 42i | *** |
| 42j | * | 42k | ** |
| 421 | * | 42m | *** |
| 42n | *** | 420 | *** |
| 42p | **** | 42q | * |
| 42r | *** | 42s | **** |
| 42t | *** | 43 | ** |
| 44a | *** | 44b | **** |
| 44c | *** | 44d | **** |
| 44e | ** | 45 | **** |
| 46a | **** | 46b | *** |
| 46c | **** | 46d | **** |
| 46e | **** | 47 | *** |
| 48 | **** | 49 | * |
| 50 | **** | 51a | **** |
| 51b | **** | 52 | **** |
| 53 | *** | 54 | * |
| 55 | ** | 56 | * |
| 57 | * | 58 | * |
| 59a | ** | 59b | ** |
| 59c | * | 59d | ** |
| 59e | * | 59f | * |
| 60a | * | 60b | * |
| 60c | * | 60d | * |
| 60€ | * | 61 | * |
| 62 | * | 63 | * |
| 64 | ** | 65a | **** |
| 65b | *** | 65c | **** |
| 65d | * | 65e | **** |
| 66 | *** | 67 | **** |
| 68 | ** | 69 | * |
| 70 | * | 71a | *** |
| 71 | ** | 72 | * |
| 73 | **** | 74a | **** |
| 74b | * | 74c | *** |
| 74d | * | 74e | **** |
| 74f | **** | 74g | **** |
| 74h | **** | 74i | **** |
| 75 | **** | 76aa | *** |
| 76ab | **** | 76ac | **** |
| 76ad | **** | 76ae | **** |
| 76af | **** | 76ag | *** |
| 76ah | **** | 76ai | **** |
| 76aj | **** | 76ak | *** |
| 76al | **** | 76am | **** |
| 76an | **** | 76ao | **** |
| 76ap | *** | 76aq | **** |
| 76ar | **** | 76as | **** |
| 76at | **** | 76au | **** |
| 76av | **** | 76aw | **** |
| 76ax | **** | 76ay | **** |
| 76az | **** | 76ba | **** |
| 76bb | **** | 76bc | **** |
| 76bd | **** | 76be | *** |
| 77 | *** | 78a | **** |
| 78b | * | 79 | **** |
| 80a | **** | 80b | **** |
| 80c | *** | 80d | **** |
| 80e | **** | 80f | *** |
| 80g | ** | 80h | **** |
| 80i | *** | 80j | **** |
| 80k | **** | 80l | **** |
| 80m | **** | 80n | **** |
| 80o | **** | 80p | **** |
| 80q | **** | 80r | **** |
| 80s | **** | 80t | **** |
| 80u | **** | 80v | **** |
| 80w | *** | 80x | *** |
| 80y | **** | 81 | **** |
| 82 | **** | 83 | **** |
| 84 | **** | 85 | **** |
| 86 | **** | 87 | **** |
| 88 | **** | 89 | **** |
| 90 | **** | 91a | **** |
| 91b | *** | 91c | **** |
| 91d | **** | 91e | **** |
| 91f | **** | 91g | *** |
| 91h | **** | 91i | **** |
| 91j | **** | 91k | **** |
| 91l | **** | 91m | **** |
| 91n | **** | 91o | **** |
| 91p | **** | 91q | **** |
| 91r | **** | 91s | **** |
| 92 | * | 93a | **** |
| 93b | *** | 93c | *** |
| 93d | **** | 93e | *** |
| 93f | **** | 93g | **** |
| 93h | **** | 93i | **** |
| 93j | ** | 93k | **** |
| 93l | **** | 93m | **** |
| 93n | **** | 93o | **** |
| 93p | * | 93q | **** |
| 93r | **** | 94 | ** |
| 95 | ** | 96a | *** |
| 96b | *** | 96c | * |
| 96d | * | 97 | **** |

Note:
****: ≤50 nM;
***: >50 nM, <200 nM;
**: ≥200 nM, ≤500 nM;
*: >500 nM

1-29. (canceled)

30. A compound or a pharmaceutically acceptable salt of formula (I) or a stereoisomer or an isotopic isomer thereof, 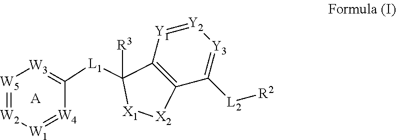
wherein, X₁ represents (CR^{L}R^{L′})ₘ;
wherein, X₂ represents (CR^{T}R^{T′})ₙ;
wherein, W₁ independently represents N;
wherein, W₂ independently represents CR^{W2} or N;
wherein, W₃ independently represents CR^{W3} or N;
wherein, W₄ independently represents CR^{W4} or N;
wherein, W₅ independently represents CR¹ or N;
wherein, Y₁ independently represents CR^{Y1};
wherein, Y₂ independently represents CR^{Y2};
wherein, Y₃ independently represents CR^{Y3};
wherein, each R^{W2}, R^{W3}, R^{W4}, R^{Y1}, R^{Y2}, R^{Y3} independently represents hydrogen, C₁-C₆ alkyl, halogen, —OR^{a}, —CN, —NR^{a}R^{b}, —CR^{a}R^{b}NR^{a}R^{b}, C₁-C₆ haloalkyl;
or W¹ and W² together with the atom which they are attached to form a 5-6 membered saturated or unsaturated ring fused to ring A, and the said ring may optionally contain 0, 1, 2 or 3 heteroatoms selected from O, N, or S;
or W₁ and W₄ together with the atom which they are attached to form a 5-6 membered saturated or unsaturated ring fused to ring A, and the said ring may optionally contain 0, 1, 2 or 3 heteroatoms selected from O, N, or S;
wherein, L₁ represents —O—, —NR^{a}—;
wherein, L₂ represents —NR^{a}—, —NR^{a}C(O)NR^{b}—, —NR^{a}C(O)—, or —C(O)NR^{a}—;
Wherein, R¹ represents hydrogen, deuterium, C₁-C₆ alkyl, deuterated C₁-C₆ alkyl, halogen, —OR^{a}, —SR^{a}, —P(O)R^{a}R^{b}, —CN, —S(O)₂R^{a}, —S(O)R^{a}, —SF₅, —NR^{a}R^{b}, C₁-C₆ haloalkyl, C₁-C₆ haloalkoxy, C₁-C₆ haloalkylthio, hydroxy (C₁-C₆alkyl); or R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl, which are substituted by 0-4 substituents that selected from deuterium, halogen, C₁-C₆ alkyl, C₃-C₆ heterocycloalkyl, C₂-C₆ alkenyl, C₂-C₆ alkynyl, C₃-C₆ cycloalkyl, —OR^{a}, oxo, hydroxy (C₁-C₆alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}; and when R¹ is a ring, any two adjacent substituents on the ring may combine to form a ring, and the said ring may optionally contain 0, 1 or 2 heteroatoms selected from O, N, or S; further, the said ring may optionally be substituted by halogen, hydroxy or C₁-C₆ alkyl;
or W₅ and W₂ together with the atom which they are attached to form a 5-6 membered saturated or unsaturated ring fused to ring A, and the said ring may optionally contain 0, 1, 2 or 3 heteroatoms selected from O, N, or S; and further, the said ring may optionally substituted by 0, 1 or 2 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, R² represents C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, —CR^{a}R^{b}C₆-C₁₀ aryl, —CR^{a}R^{b}(5-10 membered heteroaryl); which are substituted by 0-4 substituents selected from deuterium, halogen, C₁-C₆alkyl, C₂-C₆ alkenyl, C₂-C₆ alkynyl, C₃-C₆ cycloalkyl, C₃-C₆ heterocycloalkyl, C₆-C₁₀ aryl, 5-10 membered heteroaryl, —OR^{a}f, oxo, hydroxy (C₁-C₆alkyl), —NR^{a}R^{b}, —CR^{a}R^{b}NR^{a}R^{b}, —CN, C₁-C₆ haloalkyl, C₁-C₆ haloalkoxy, C₃-C₆ halocycloalkyl, C₃-C₆ haloheterocycloalkyl, C₁-C₆ alkyl-substituted C₃-C₆ heterocycloalkyl, —SO₃R^{a}, —SR^{a}, —S(O)₂R^{a}, —S(O)R^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}; and when R² is a ring, any two adjacent substituents on the ring may combine to form a ring, and the said ring may optionally contain 0, 1 or 2 heteroatoms selected from O, N, or S; and further, the said ring may optionally be substituted by halogen, hydroxy or C₁-C₆ alkyl;
wherein, R³ represents hydrogen, halogen, C₁-C₆ haloalkyl, C₁-C₆ alkyl, or hydroxy;
or R³ and W₃ together with the atom which they are attached to form a 5-7 membered saturated or unsaturated ring fused to ring A, and the said ring may optionally contain 0, 1 or 2 heteroatoms selected from O, N, or S; and further, the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, each R^{a} and R^{b} independently represents hydrogen, deuterium, halogen, C₁-C₆ alkyl, C₁-C₆ haloalkyl, hydroxy (C₁-C₆ alkyl), (CH₃)₂N—(C₁-C₆ alkyl), C₃-C₁₀ cycloalkyl; or R^{a} and R^{b} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, each R^{L} and R^{L′} independently represents hydrogen, deuterium, C₁-C₆ alkyl, C₁-C₆ haloalkyl, hydroxy (C₁-C₆alkyl), C₃-C₁₀ cycloalkyl;
wherein, each R^{T} and R^{T′} independently represents hydrogen, deuterium, halogen, C₁-C₆alkyl, C₁-C₆ haloalkyl, hydroxy (C₁-C₆ alkyl), C₃-C₁₀ cycloalkyl;
wherein, o, m, n represents the integer of 1, 2 or 3.

31. The compound or the pharmaceutically acceptable salt according to claim 30, or the stereoisomer or isotopic isomer thereof, wherein, 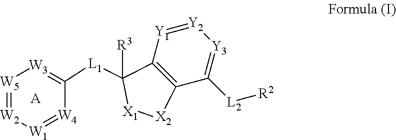
wherein, X₁ represents O, S, NR^{a}, (CR^{L}R^{L′})ₘ, C(O);
wherein, X₂ represents O, S, NR^{a}f, (CR^{T}R^{T′})ₙ, C(O);
wherein, W₁ independently represents N;
wherein, W₂ independently represents CR^{W2} or N;
wherein, W₃ independently represents CR^{W3} or N;
wherein, W₄ independently represents CR^{W4} or N;
wherein, W₅ independently represents CR¹ or N;
wherein, Y₁ independently represents CR^{Y1};
wherein, Y₂ independently represents CR^{Y2};
wherein, Y₃ independently represents CR^{Y3};
wherein, each R^{W2}, R^{W3}, R^{W4}, R^{Y1}, R^{Y2}, R^{Y3} independently represents hydrogen, C₁-C₆ alkyl, halogen, —OR^{a}, —CN, —NR^{a}R^{b}, —CR^{a}R^{b}NR^{a}R^{b}, C₁-C₆ haloalkyl;
or W₁ and W₂ together with the atom which they are attached to form a 5-6 membered saturated or unsaturated ring fused to ring A, and the said ring may optionally contain 0, 1, 2 or 3 heteroatoms selected from O, N, or S;
or W₁ and W₄ together with the atom which they are attached to form a 5-6 membered saturated or unsaturated ring fused to ring A, and the said ring may optionally contain 0, 1, 2 or 3 heteroatoms selected from O, N, or S;
wherein, L₁ represents —O—, or —NR^{a};
wherein, L₂ represents —NR^{a}—, —NR^{a}C(O)NR^{b}—, —NR^{a}C(O)—, or —C(O)NR^{a}—;
Wherein, R¹ represents hydrogen, deuterium, C₁-C₆ alkyl, deuterated C₁-C₆ alkyl, halogen, —OR^{a}, —SR^{a}, —P(O)R^{a}R^{b}, —CN, —S(O)₂R^{a}, —S(O)R^{a}, —SF₅, —NR^{a}R^{b}, C₁-C₆ haloalkyl, C₁-C₆ haloalkoxy, C₁-C₆ haloalkylthio, hydroxy (C₁-C₆alkyl); or R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl, which are substituted by 0-4 substituents that selected from deuterium, halogen, C₁-C₆ alkyl, C₂-C₆ alkenyl, C₂-C₆ alkynyl, C₃-C₆ cycloalkyl, C₃-C₆ heterocycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
or W₅ and W₂ together with the atom which they are attached to form a 5-6 membered saturated or unsaturated ring fused to ring A, and the said ring may optionally contain 0, 1 or 2 heteroatoms selected from O, N, or S; and further, the said ring may optionally substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, R² represents C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, —CR^{a}R^{b}C₆-C₁₀ aryl, —CR^{a}R^{b} (5-10 membered heteroaryl); which are substituted by 0-4 substituents selected from deuterium, halogen, C₁-C₆alkyl, C₂-C₆ alkenyl, C₂-C₆ alkynyl, C₃-C₆ cycloalkyl, C₃-C₆ heterocycloalkyl, C₆-C₁₀ aryl, 5-10 membered heteroaryl, —OR^{a}, oxo, hydroxy (C₁-C₆alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —S(O)₂R^{a}, —S(O)R^{a}, —SF₅, —C(O)R^{a}, —C(O)OR^{a}f, —OC(O)R^{a}, —OC(O)NR^{a}R^{b}, —NR^{a}COR^{b} or —CONR^{a}R^{b};
wherein, R³ represents hydrogen, halogen, C₁-C₆ haloalkyl, C₁-C₆ alkyl, or hydroxy;
or R³ and W₃ together with the atom which they are attached to form a 5-7 membered saturated or unsaturated ring fused to ring A, and the said ring may optionally contain 0, 1 or 2 heteroatoms selected from O, N, or S; and further, the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b};
wherein, each R^{a} and R^{b} independently represents hydrogen, deuterium, halogen, C₁-C₆ alkyl, C₁-C₆ haloalkyl, hydroxy (C₁-C₆ alkyl), C₃-C₁₀ cycloalkyl; or R^{a} and R^{b} together with the carbon atom which they are attached to form a 3-6 membered ring, and the said ring may optionally contain 0, 1 or 2 heteroatoms selected from O, N, or S; and further, the said ring may optionally be substituted by halogen, hydroxy or C₁-C₆ alkyl;
wherein, each R^{L} and R^{L′} independently represents hydrogen, deuterium, C₁-C₆ alkyl, C₁-C₆ haloalkyl, hydroxy (C₁-C₆alkyl), C₃-C₁₀ cycloalkyl;
wherein, each R^{T} and R^{T′} independently represents hydrogen, deuterium, halogen, C₁-C₆alkyl, C₁-C₆ haloalkyl, hydroxy (C₁-C₆ alkyl), C₃-C₁₀ cycloalkyl;
wherein, o, m, n represents the integer of 1, 2 or 3.

32. The compound or the pharmaceutically acceptable salt according to claim 30, or the stereoisomer or isotopic isomer thereof, wherein R¹ represents C₁-C₆ alkyl, halogen, —OH, O(C₁-C₆ alkyl); or R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

33. The compound or the pharmaceutically acceptable salt according to claim 31, or the stereoisomer or isotopic isomer thereof, wherein R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

34. The compound or the pharmaceutically acceptable salt according to claim 31, or the stereoisomer or isotopic isomer thereof, wherein R¹ represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, —OR^{a}, hydroxy (C₁-C₆ alkyl), —CN or C₁-C₆ haloalkyl.

35. The compound or the pharmaceutically acceptable salt according to claim 31, or the stereoisomer or isotopic isomer thereof, wherein R¹ represents 5-10 membered heteroaryl, which are substituted by 0-4 substituents selected from deuterium, halogen, C₁-C₆alkyl, —OR^{a}f, hydroxy (C₁-C₆ alkyl), —CN or C₁-C₆ haloalkyl.

36. The compound or the pharmaceutically acceptable salt according to claim 31, or the stereoisomer or isotopic isomer thereof, wherein R¹ represents pyrazolyl, triazolyl, oxazolyl, isoxazolyl, thiazolyl, imidazolyl, which are substituted by 0-4 substituents selected from deuterium, halogen, C₁-C₆ alkyl, —OR^{a}, hydroxy (C₁-C₆ alkyl), —CN or C₁-C₆ haloalkyl.

37. The compound or the pharmaceutically acceptable salt according to claim 31, or the stereoisomer or isotopic isomer thereof, wherein R¹ represent

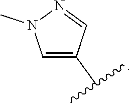 [SMILES: *c1cnn(C)c1]

38. The compound or the pharmaceutically acceptable salt according to claim 31, or the stereoisomer or isotopic isomer thereof, wherein R² represents C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, phenyl, 5-6 membered heteroaryl, —OR^{a}, oxo, hydroxy (C₁-C₆ alkyl), —NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}.

39. The compound or the pharmaceutically acceptable salt according to claim 31, or the stereoisomer or isotopic isomer thereof, wherein W₅ and W₂ combine to form a ring fused to ring A having the following structure: 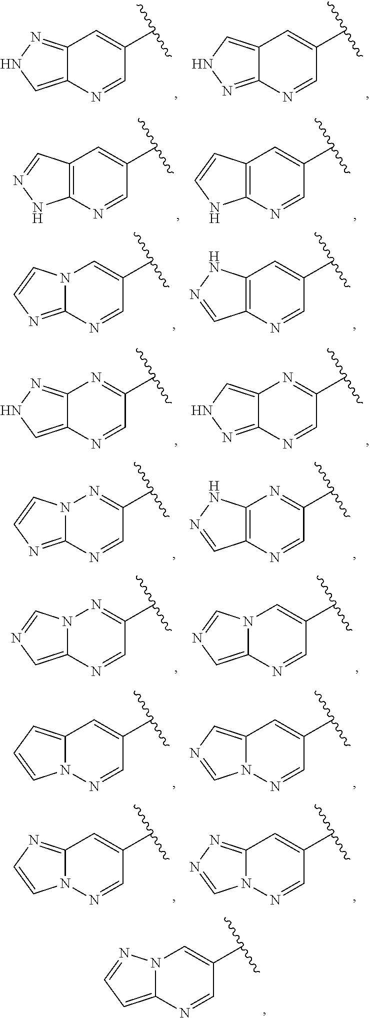
wherein the said ring may optionally be substituted by 0, 1 or 2 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), NR^{a}R^{b}, —CR^{a}R^{b}NR^{a}R^{b}, —CN, C₁-C₆ haloalkyl, C₁-C₆ haloalkoxy, —SO₃R^{a}, —SR^{a}, —SF₅, —C(O)R^{a}, —C(O)OR^{a}, —OC(O)R^{a}, —OC(O)NR^{a}R^{b}, —NR^{a}COR^{b} or —CONR^{a}R^{b}; and wherein the wavy line indicates the point of attachment with L₁.

40. The compound or the pharmaceutically acceptable salt according to claim 31, or the stereoisomer or isotopic isomer thereof, wherein W₅ and W₂ combine to form a ring fused to ring A having the following structure: 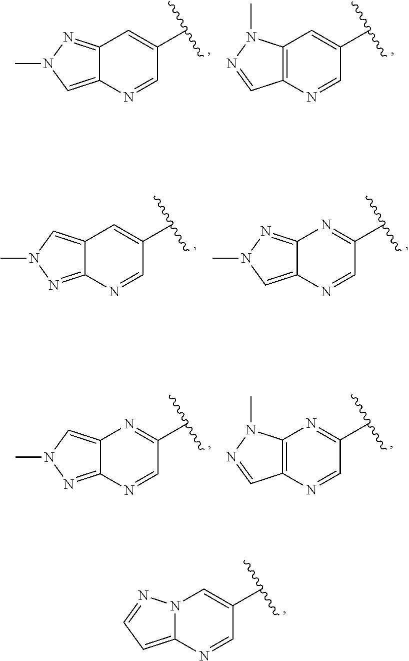
wherein the wavy line indicates the point of attachment with L₁.

41. The compound or the pharmaceutically acceptable salt according to claim 31, or the stereoisomer or isotopic isomer thereof, wherein R² represents phenyl, pyridinyl, indazolyl, indolyl, thiazolyl, isothiazolyl, imidazolyl, pyrazolyl, oxazolyl, isoxazolyl, 1,2,4-oxadiazolyl, 1,3,4-oxadiazolyl, pyridazinyl, piperazinyl, pyrazinyl, cyclohexyl, benzothienyl, 1,2-benzoisoxazolyl, benzo[d] isothiazolyl, which are substituted by 0-4 substituents selected from deuterium, halogen, C₁-C₆ alkyl, C₂-C₆ alkenyl, C₂-C₆ alkynyl, C₃-C₆ cycloalkyl, C₃-C₆ heterocycloalkyl, phenyl, 5-6 membered heteroaryl, —OR^{a}, oxo, hydroxy (C₁-C₆ alkyl), —NR^{a}R^{b}, —CR^{a}R^{b}NR^{a}R^{b}, —CN, or C₁-C₆ haloalkyl.

42. The compound or the pharmaceutically acceptable salt according to claim 31, or the stereoisomer or isotopic isomer thereof, wherein R² represents 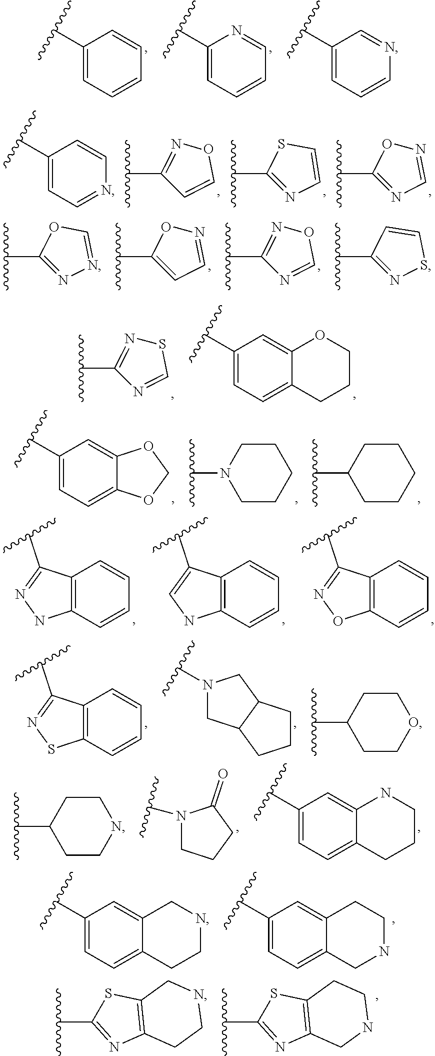
which are substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN or C₁-C₆ haloalkyl.

43. The compound or the pharmaceutically acceptable salt according to claim 31, or the stereoisomer or isotopic isomer thereof, wherein R² represents: 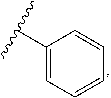 [SMILES: *c1ccccc1]
which is substituted by 0-4 substituents selected from deuterium, halogen, C₁-C₆ alkyl, C₂-C₆ alkenyl, C₂-C₆ alkynyl, C₃-C₆ cycloalkyl, —OR^{a}, oxo, hydroxy (C₁-C₆ alkyl), —NR^{a}R^{b}, —CR^{a}R^{b}NR^{a}R^{b}, —CN, or C₁-C₆ haloalkyl.

44. The compound or the pharmaceutically acceptable salt according to claim 31, or the stereoisomer or isotopic isomer thereof, wherein R³ represents hydrogen or CH₃.

45. A compound or a pharmaceutically acceptable salt thereof, wherein the compound is selected from:

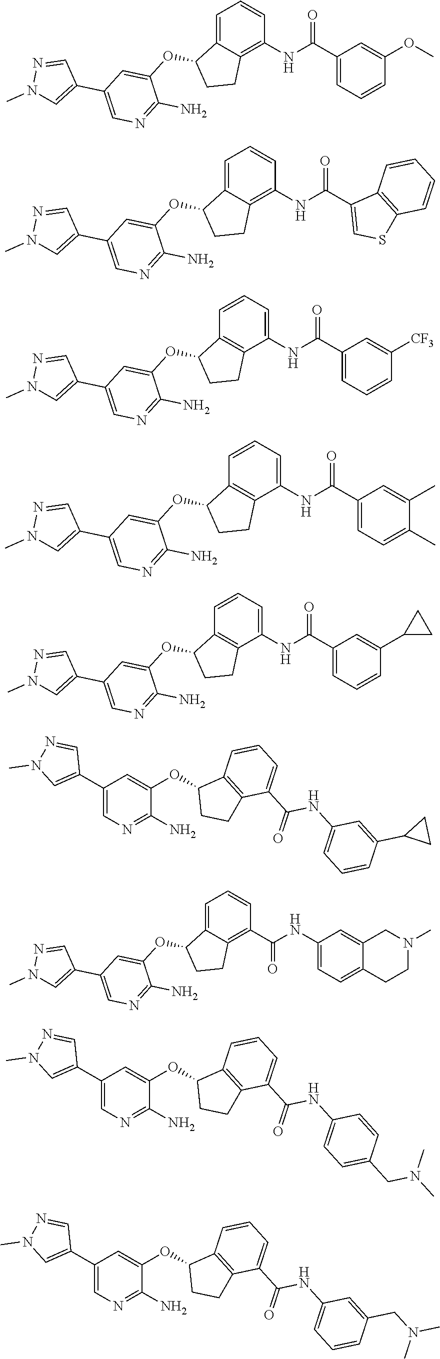

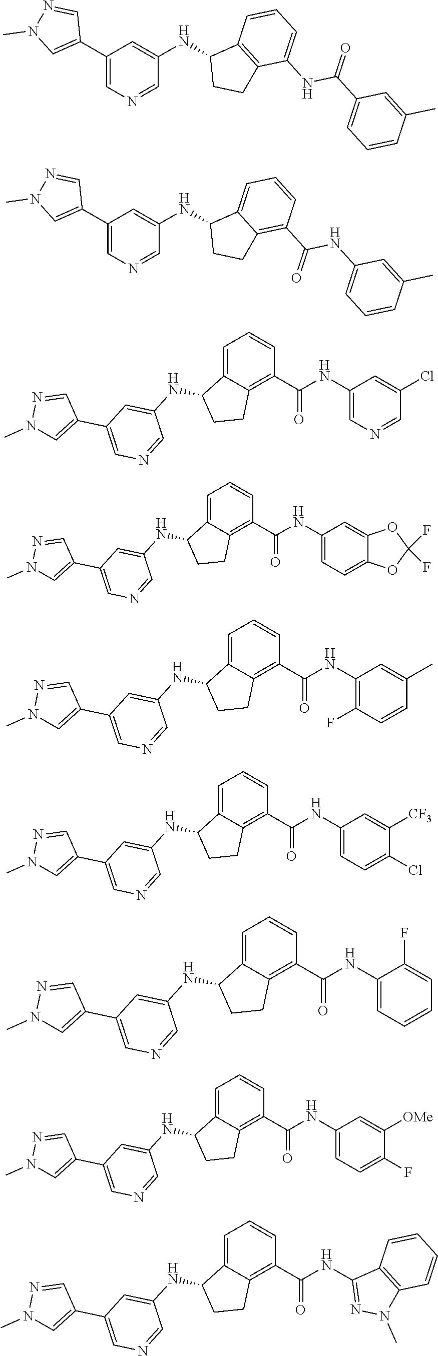

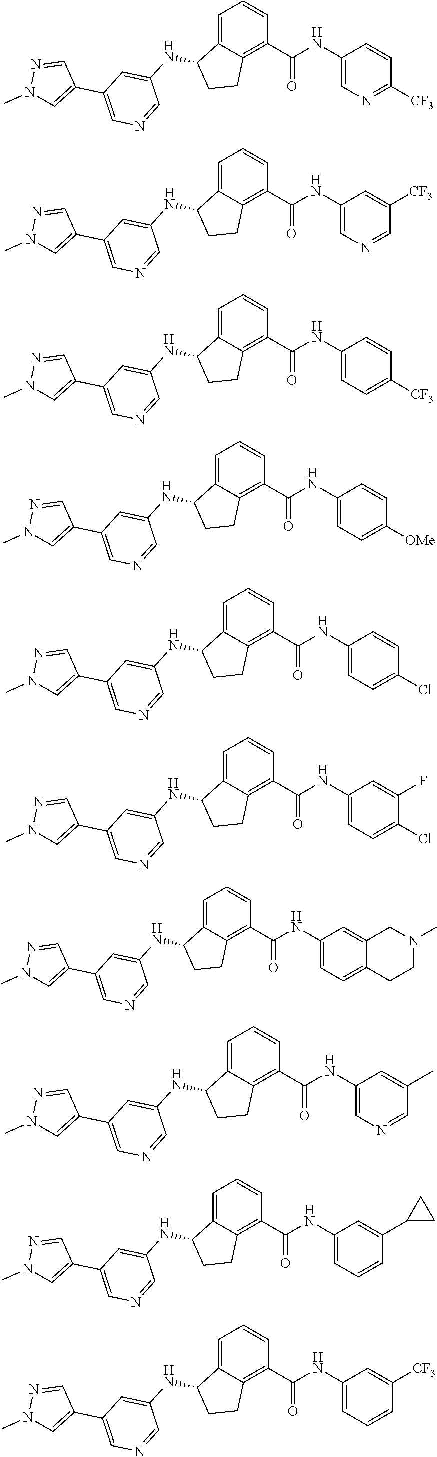

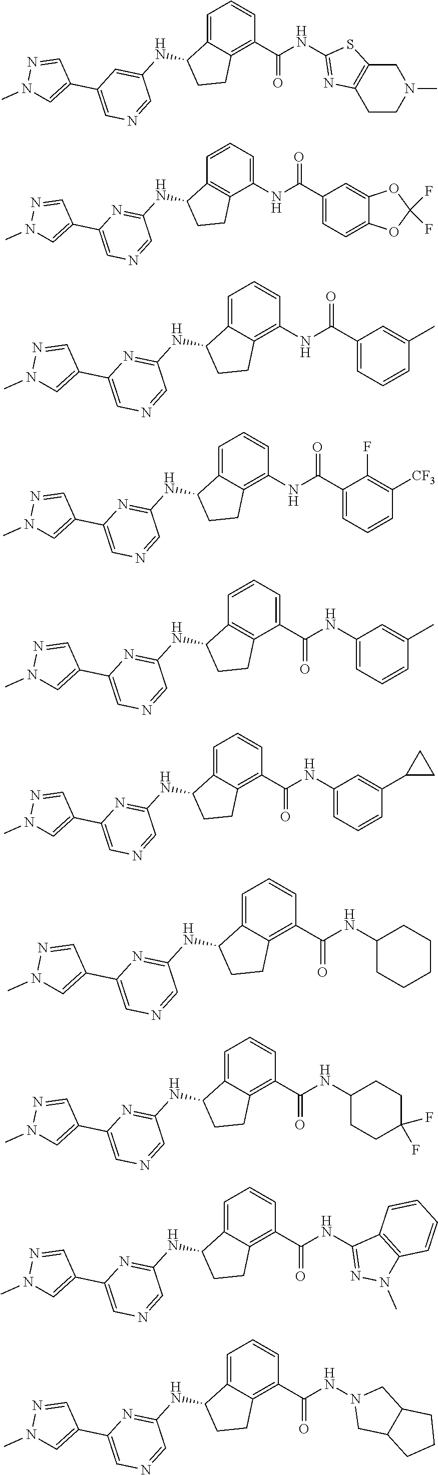

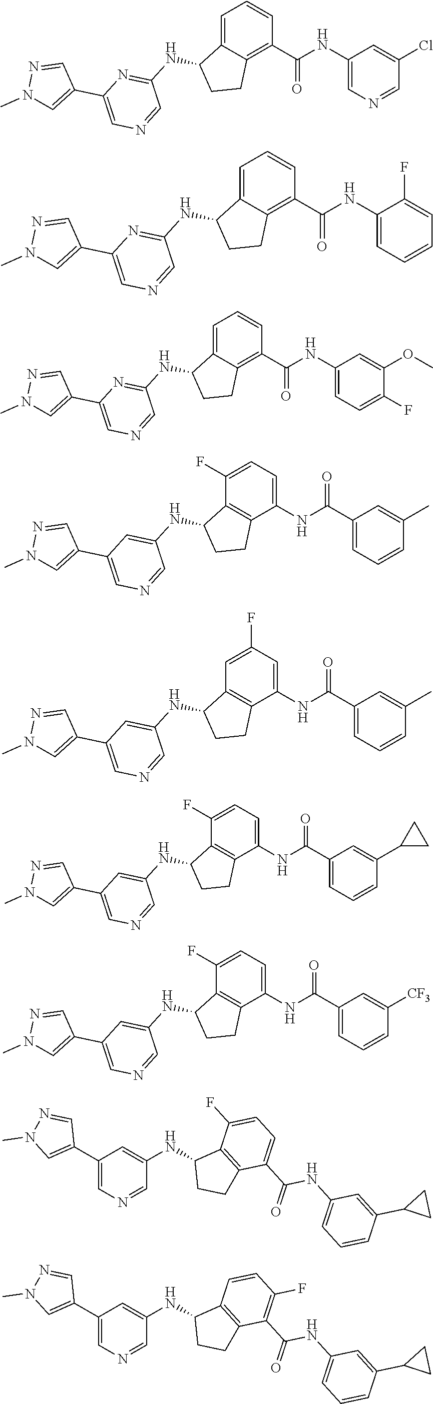

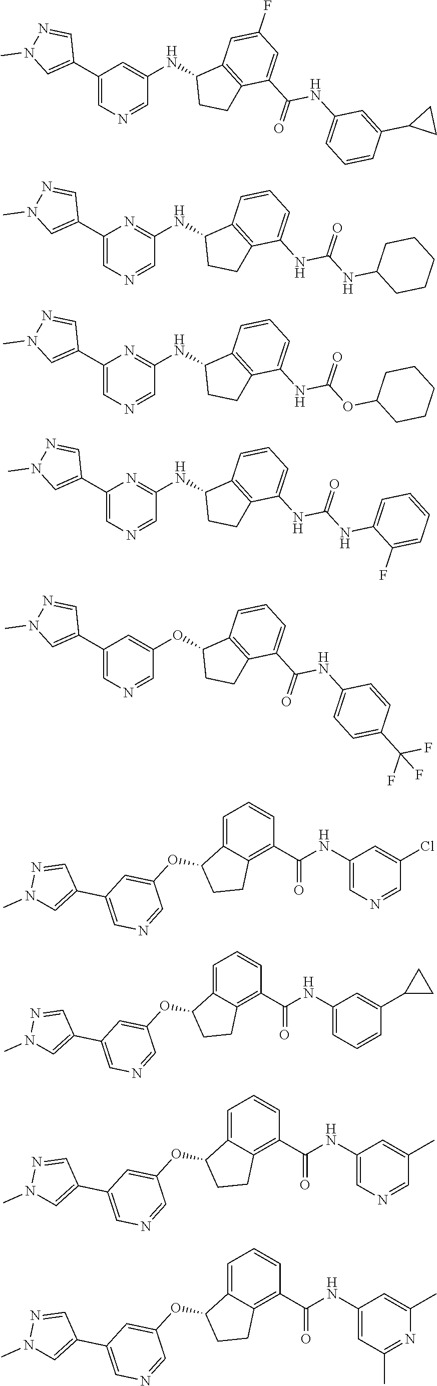

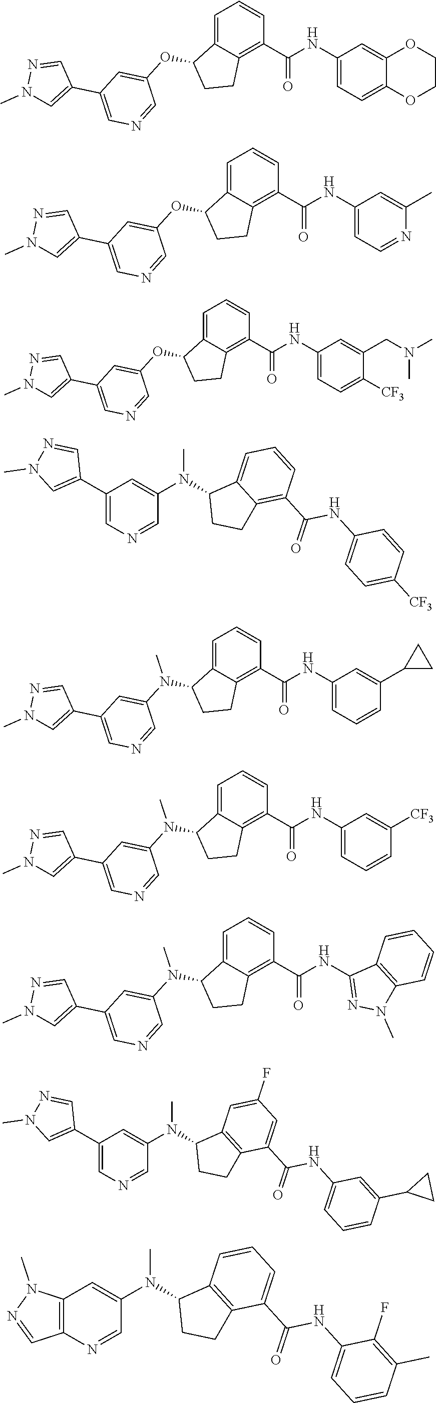

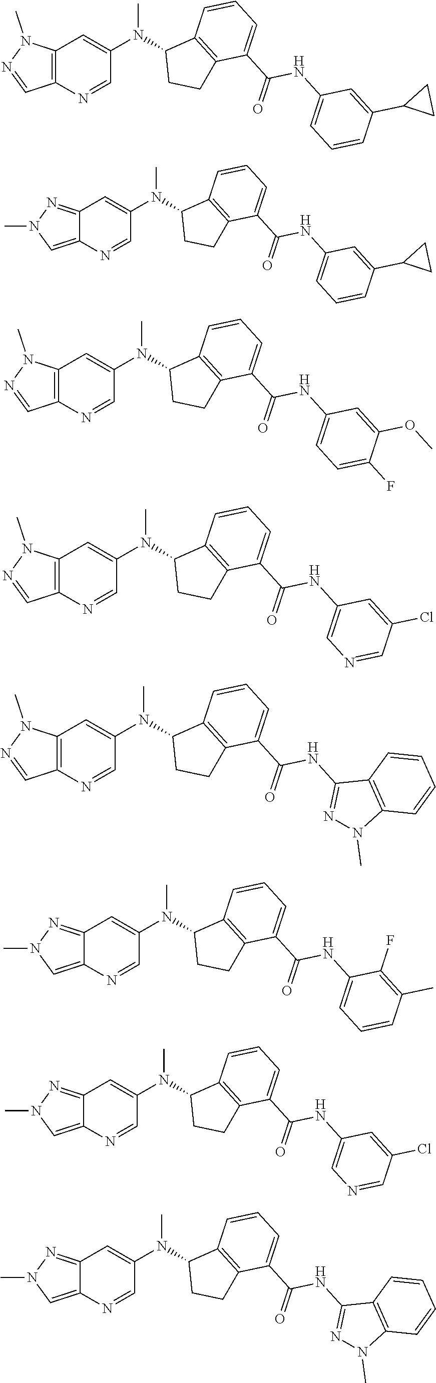

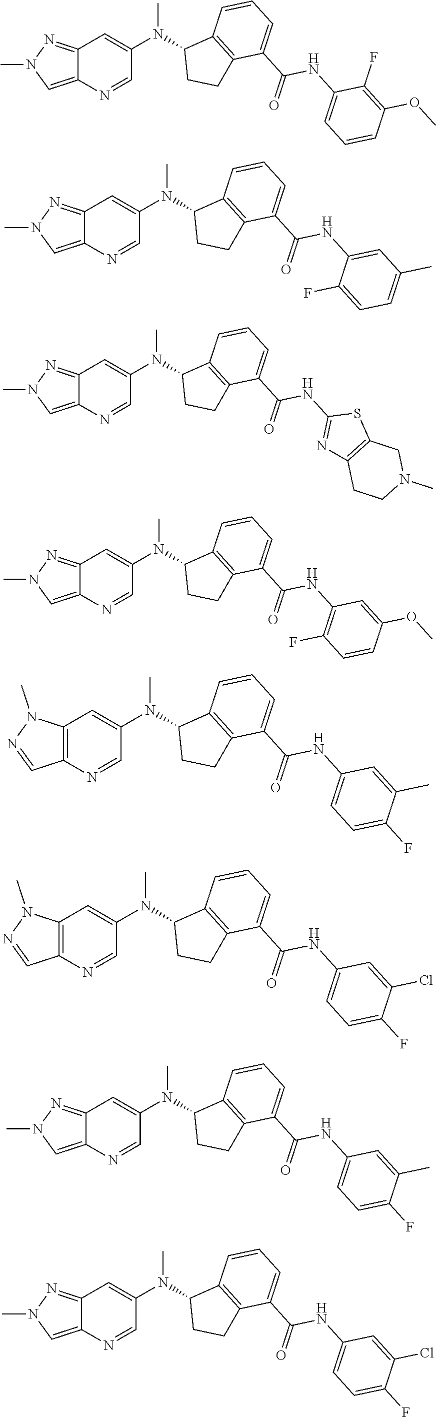

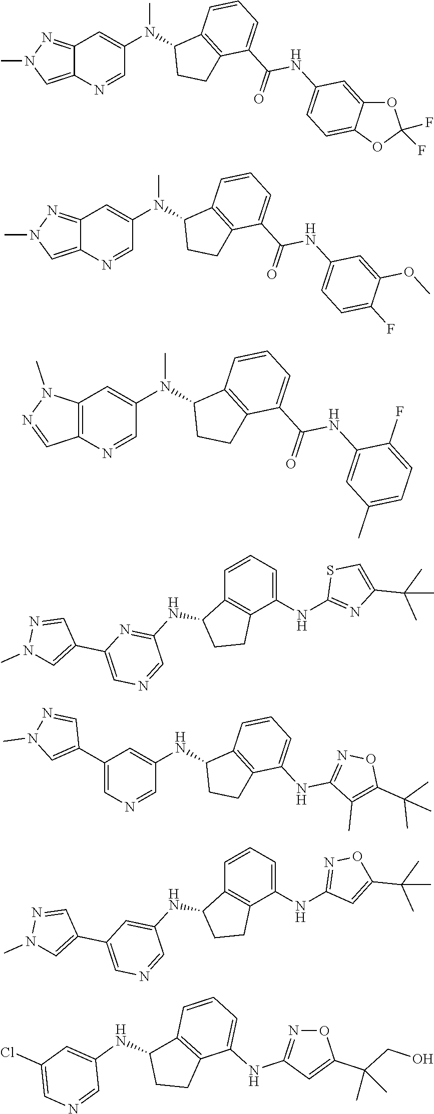

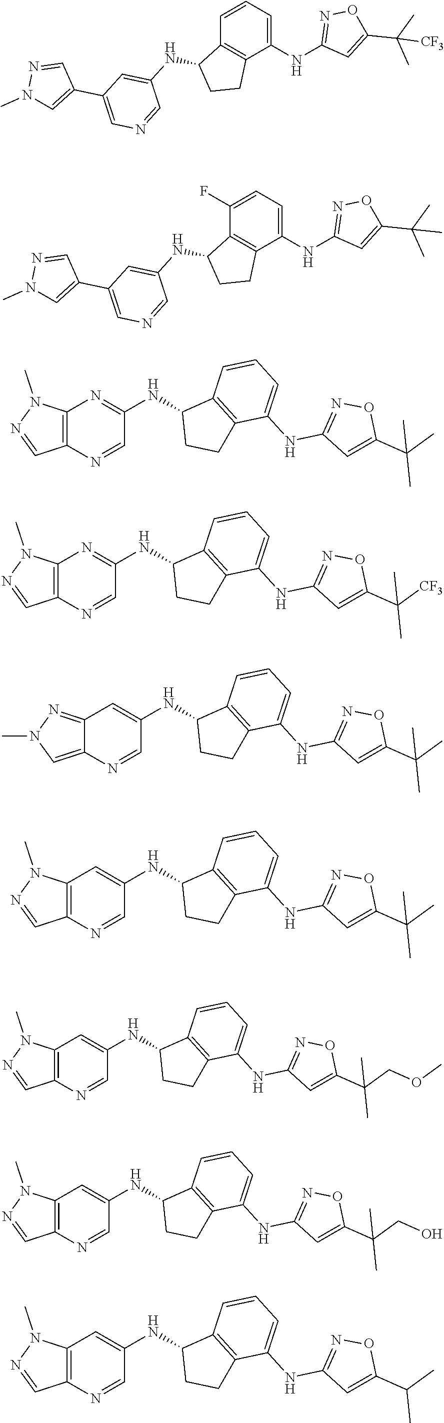

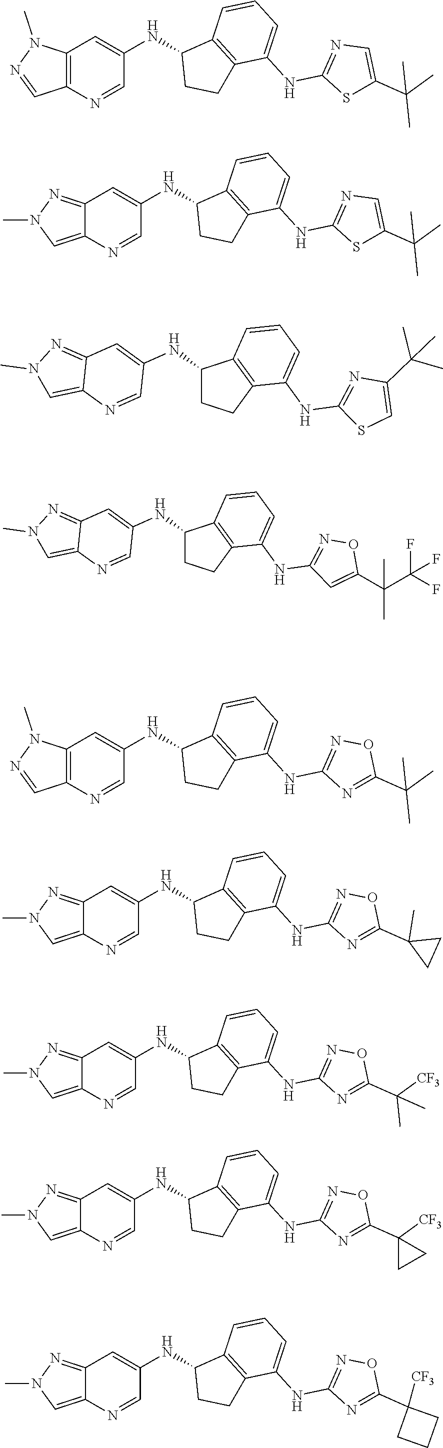

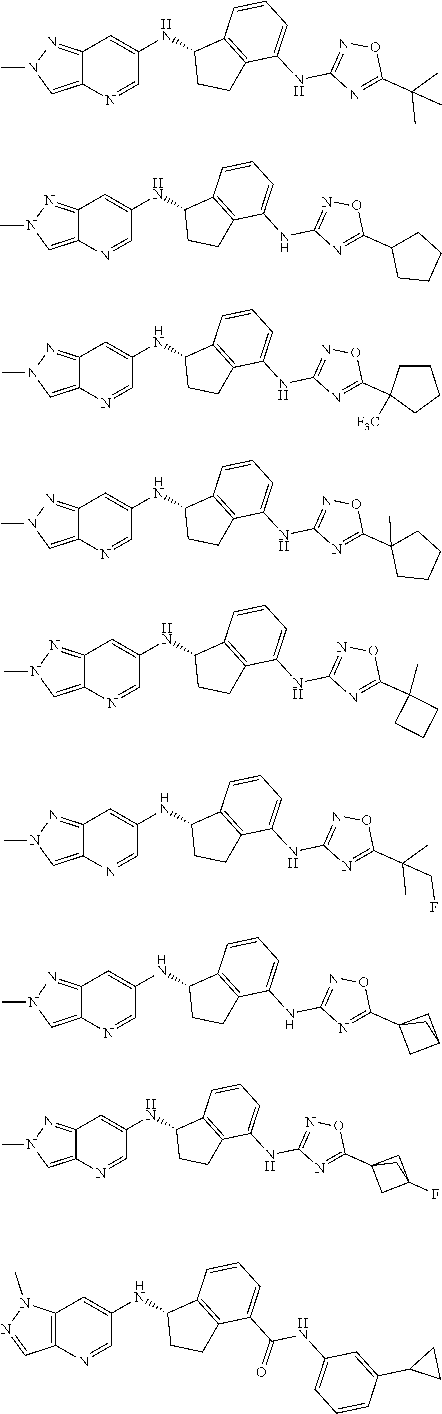

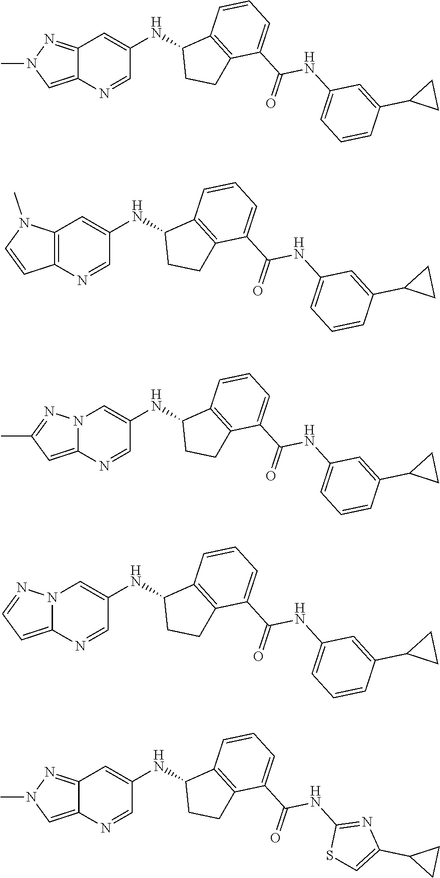 [SMILES: Cn1cc2ncc(N[C@H]3CCc4c(C(=O)Nc5cccc(C6CC6)c5)cccc43)cc2n1 ; Cn1ccc2ncc(N[C@H]3CCc4c(C(=O)Nc5cccc(C6CC6)c5)cccc43)cc21 ; Cc1cc2ncc(N[C@H]3CCc4c(C(=O)Nc5cccc(C6CC6)c5)cccc43)cn2n1 ; O=C(Nc1cccc(C2CC2)c1)c1cccc2c1CC[C@@H]2Nc1cnc2ccnn2c1 ; Cn1cc2ncc(N[C@H]3CCc4c(C(=O)Nc5nc(C6CC6)cs5)cccc43)cc2n1]

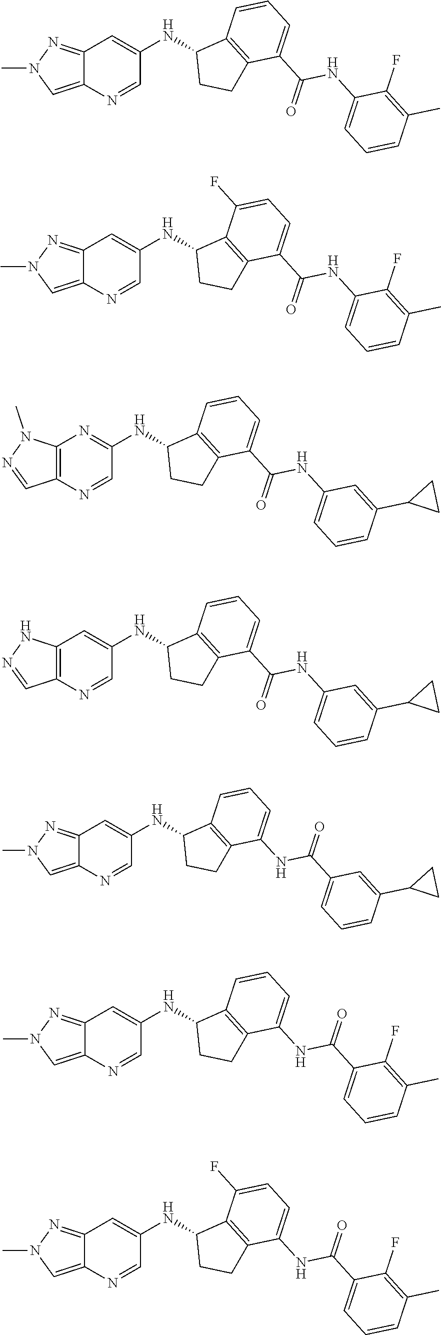

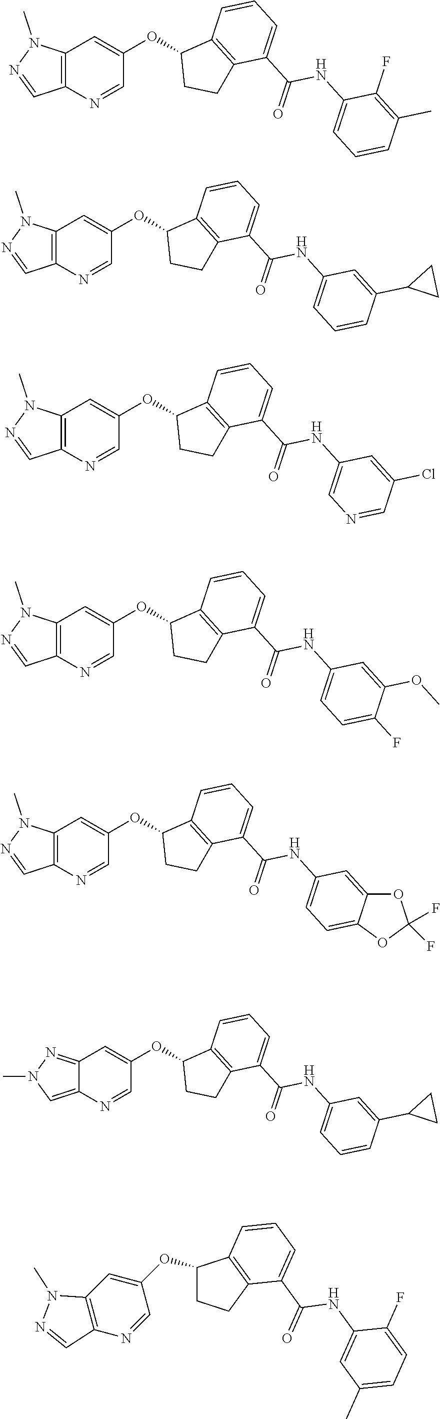

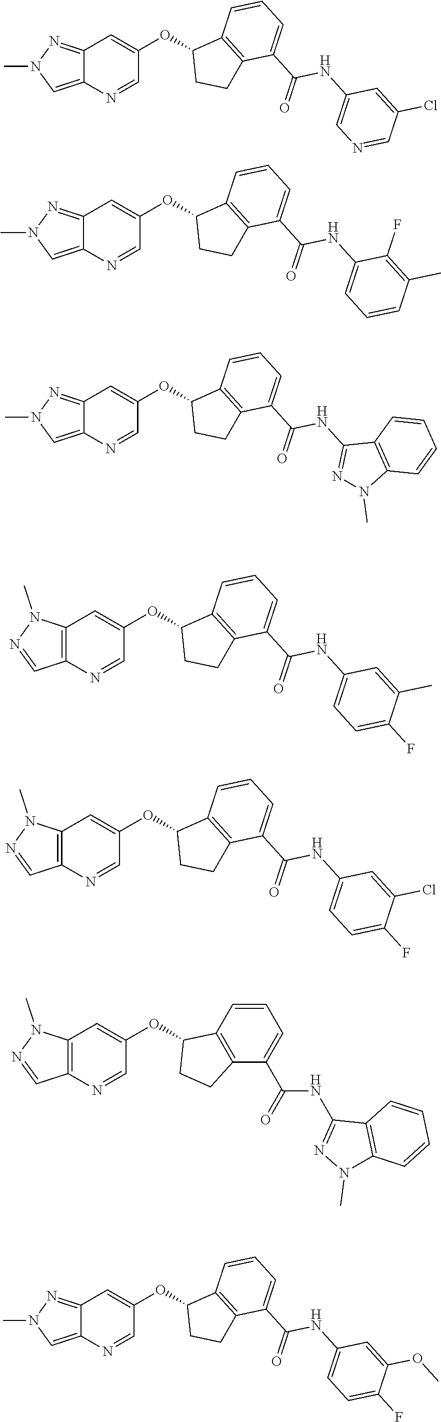

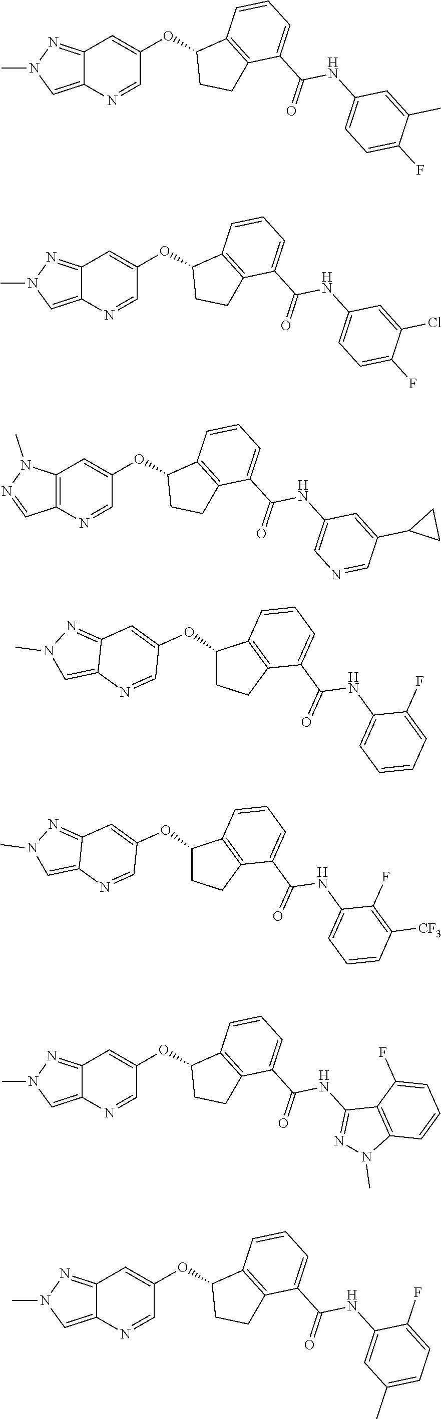

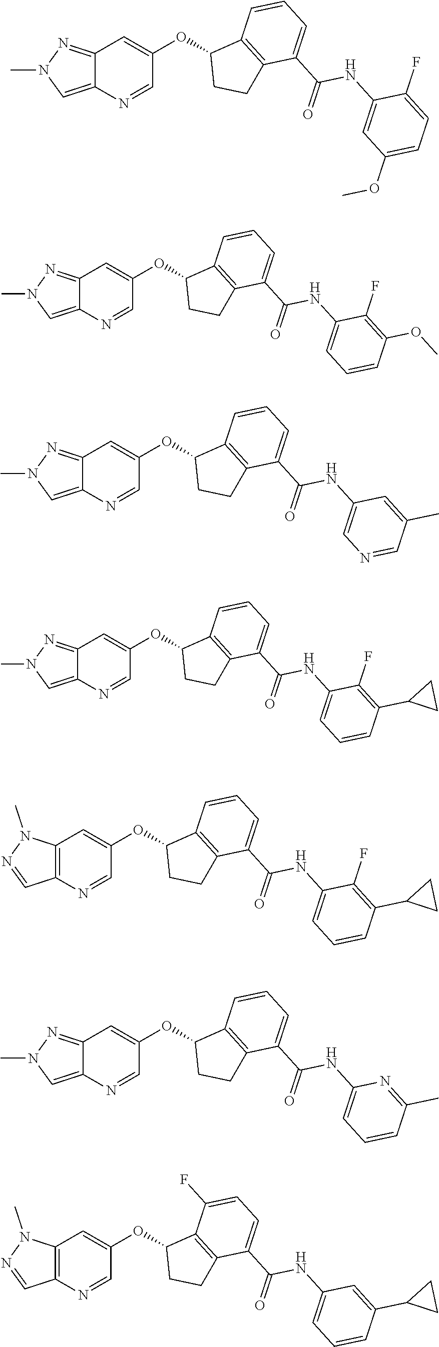

46. The compound according to claim 45 or a pharmaceutically acceptable salt thereof, wherein the compound is selected from:

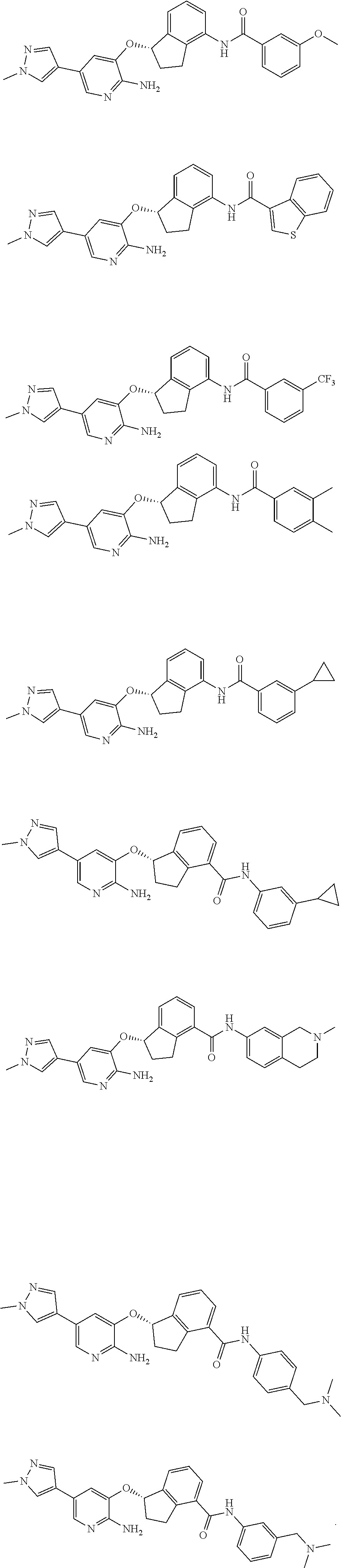

47. The compound according to claim 45 or a pharmaceutically acceptable salt thereof, wherein the compound is selected from:

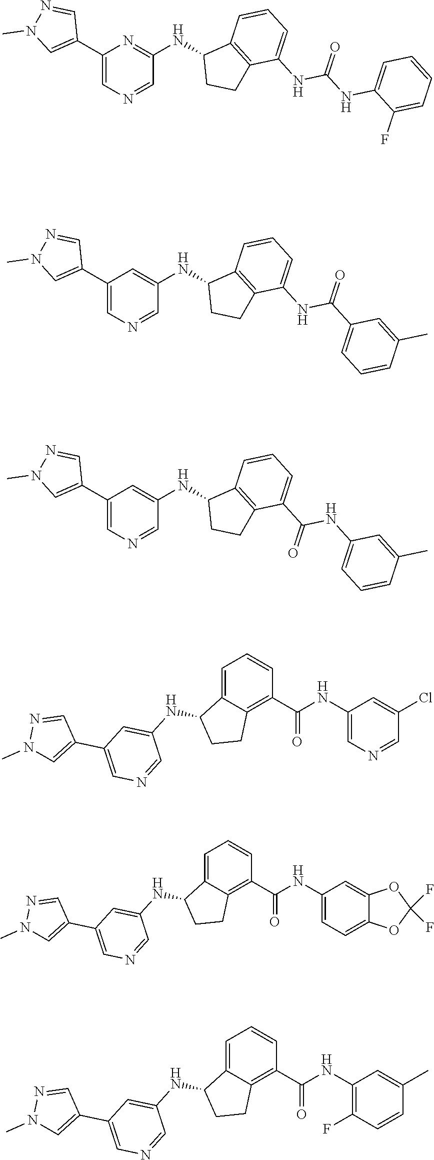

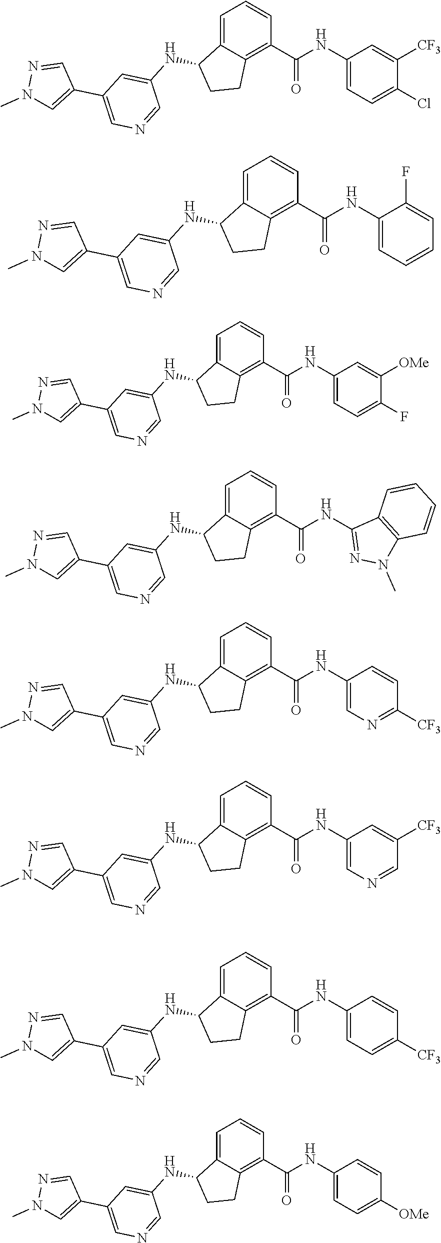

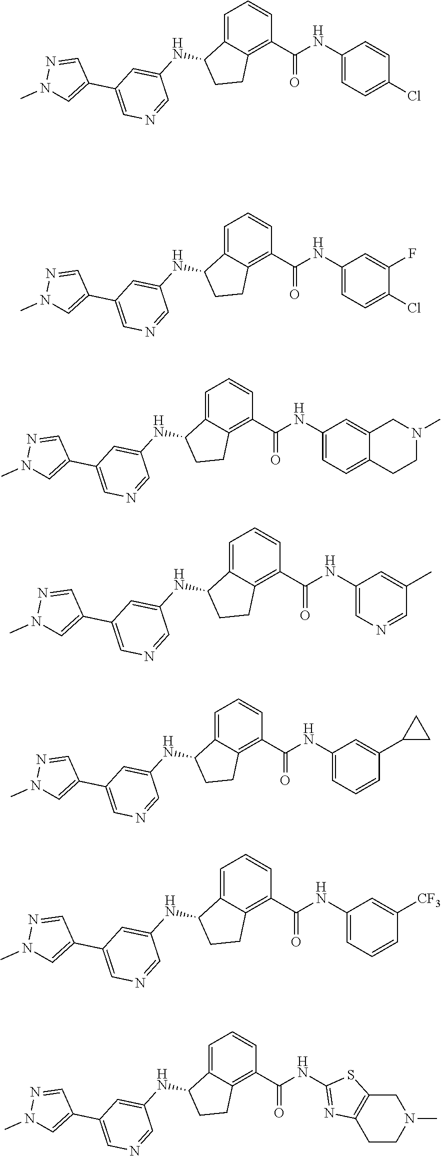

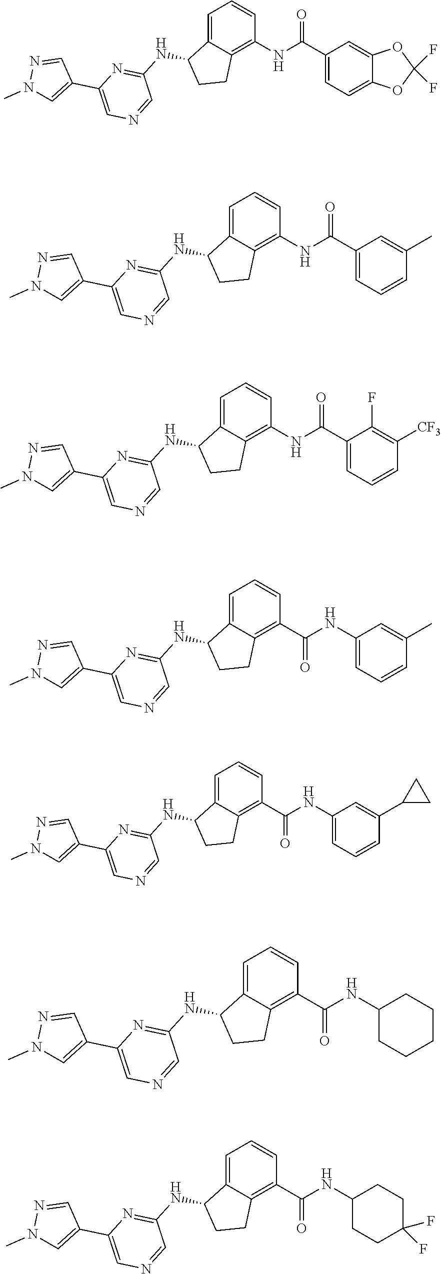

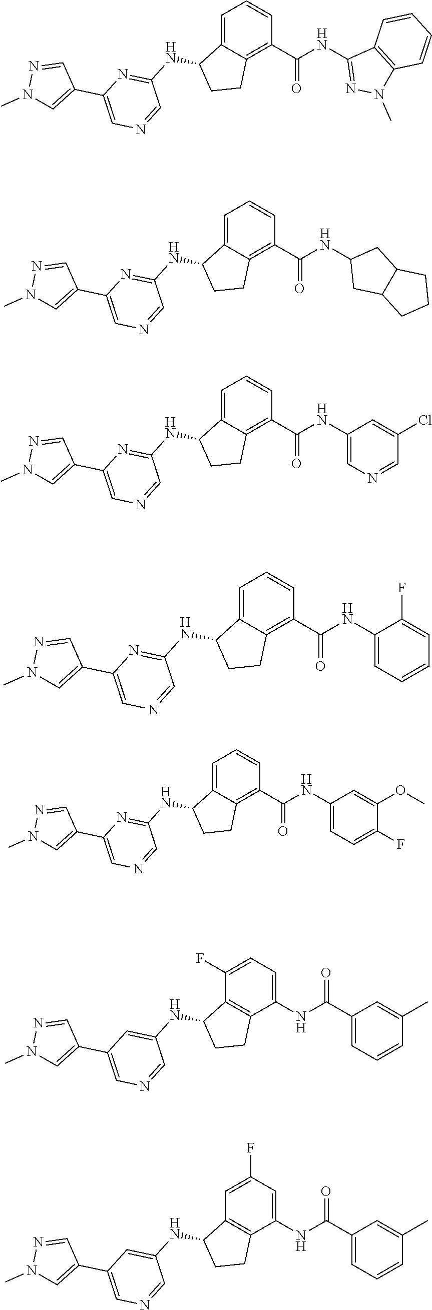

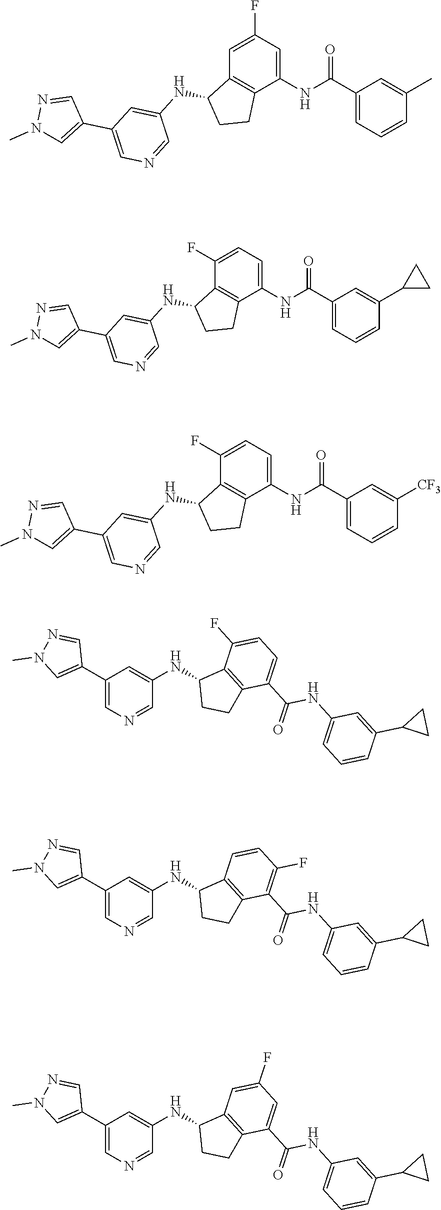

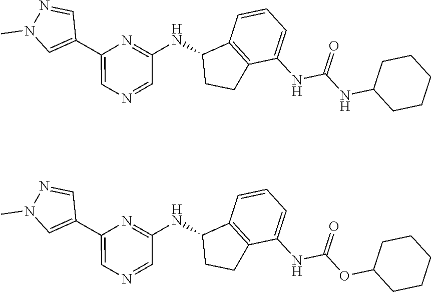 [SMILES: Cn1cc(-c2cncc(N[C@H]3CCc4c(NC(=O)NC5CCCCC5)cccc43)n2)cn1 ; Cn1cc(-c2cncc(N[C@H]3CCc4c(NC(=O)OC5CCCCC5)cccc43)n2)cn1]

48. The compound according to claim 45 or a pharmaceutically acceptable salt thereof, wherein the compound is selected from:

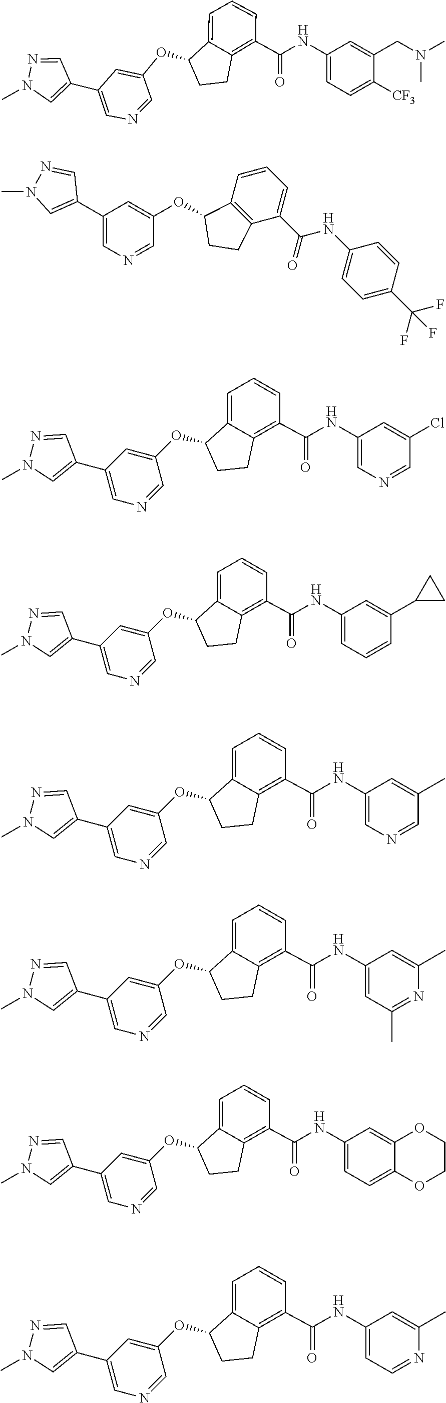

49. The compound according to claim 45 or a pharmaceutically acceptable salt thereof, wherein the compound is selected from:

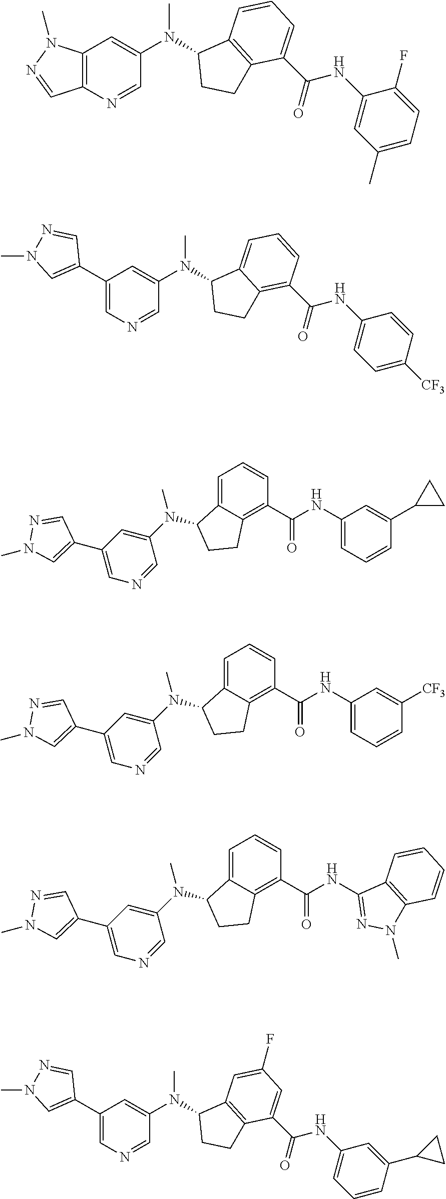 [SMILES: Cc1ccc(F)c(NC(=O)c2cccc3c2CC[C@@H]3N(C)c2cnc3cnn(C)c3c2)c1 ; CN(c1cncc(-c2cnn(C)c2)c1)[C@H]1CCc2c(C(=O)Nc3ccc(C(F)(F)F)cc3)cccc21 ; CN(c1cncc(-c2cnn(C)c2)c1)[C@H]1CCc2c(C(=O)Nc3cccc(C4CC4)c3)cccc21 ; CN(c1cncc(-c2cnn(C)c2)c1)[C@H]1CCc2c(C(=O)Nc3cccc(C(F)(F)F)c3)cccc21 ; CN(c1cncc(-c2cnn(C)c2)c1)[C@H]1CCc2c(C(=O)Nc3nn(C)c4ccccc34)cccc21 ; CN(c1cncc(-c2cnn(C)c2)c1)[C@H]1CCc2c(C(=O)Nc3cccc(C4CC4)c3)cc(F)cc21]

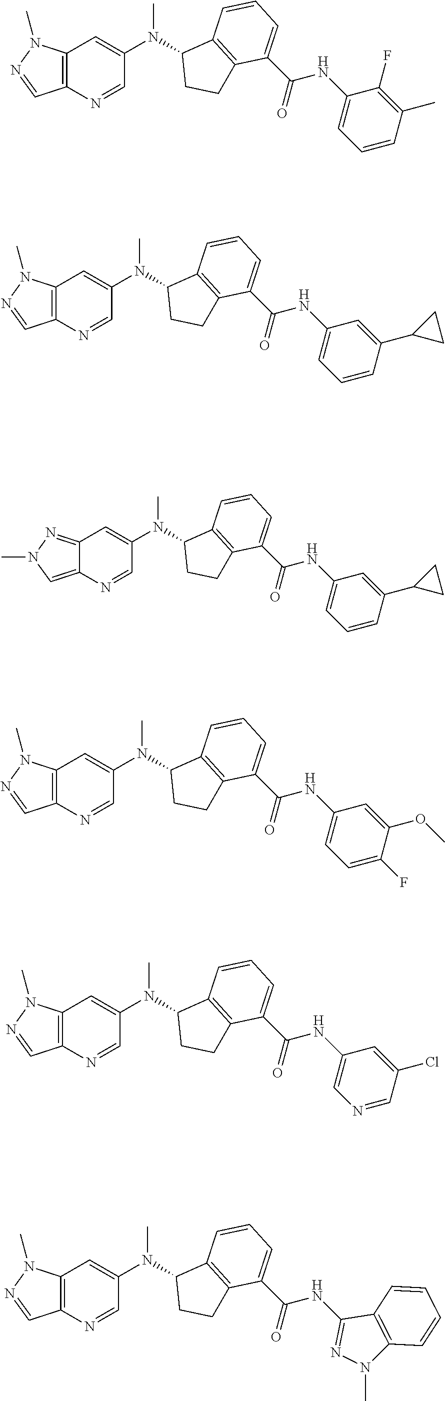

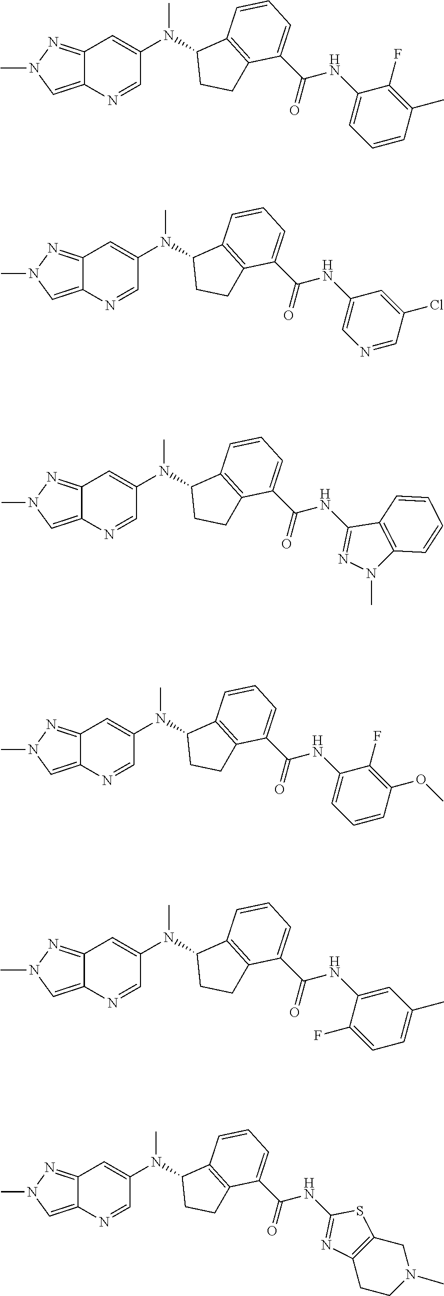

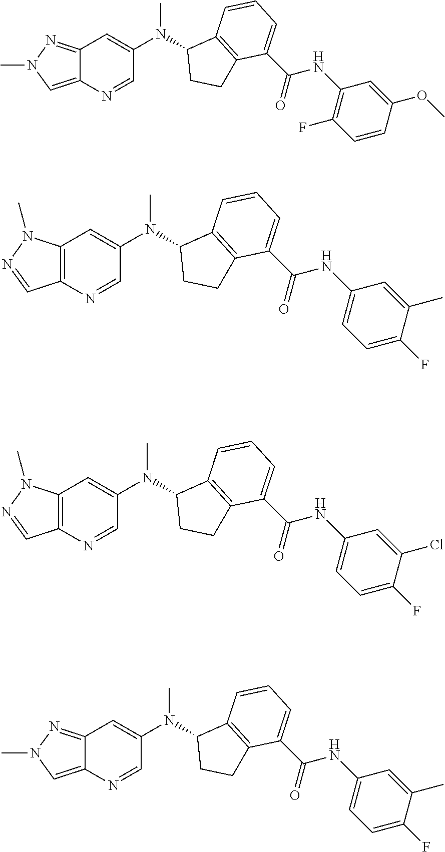

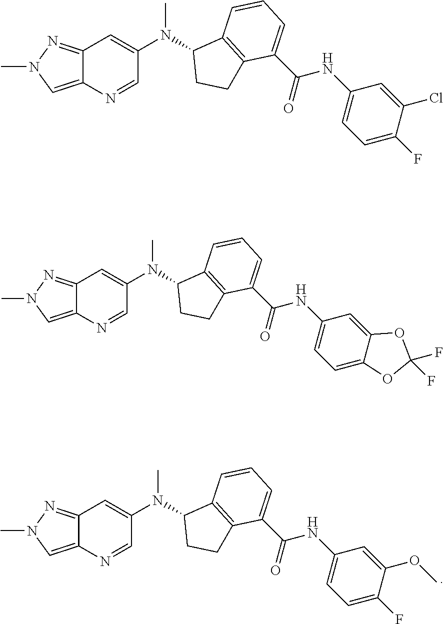

50. The compound according to claim 45 or a pharmaceutically acceptable salt thereof, wherein the compound is selected from:

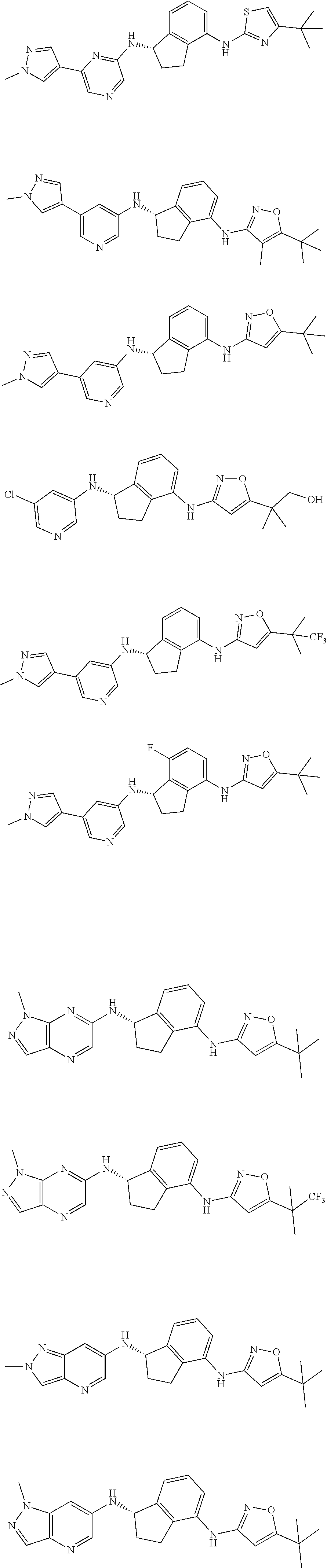

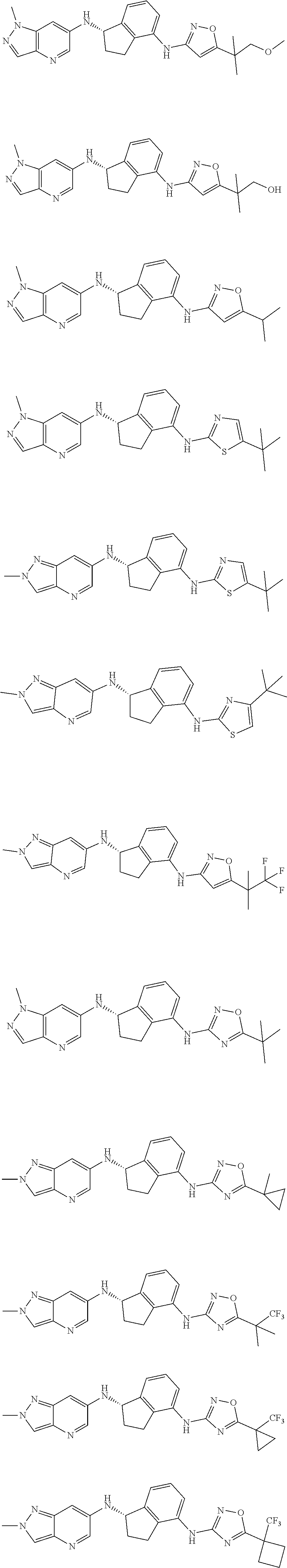

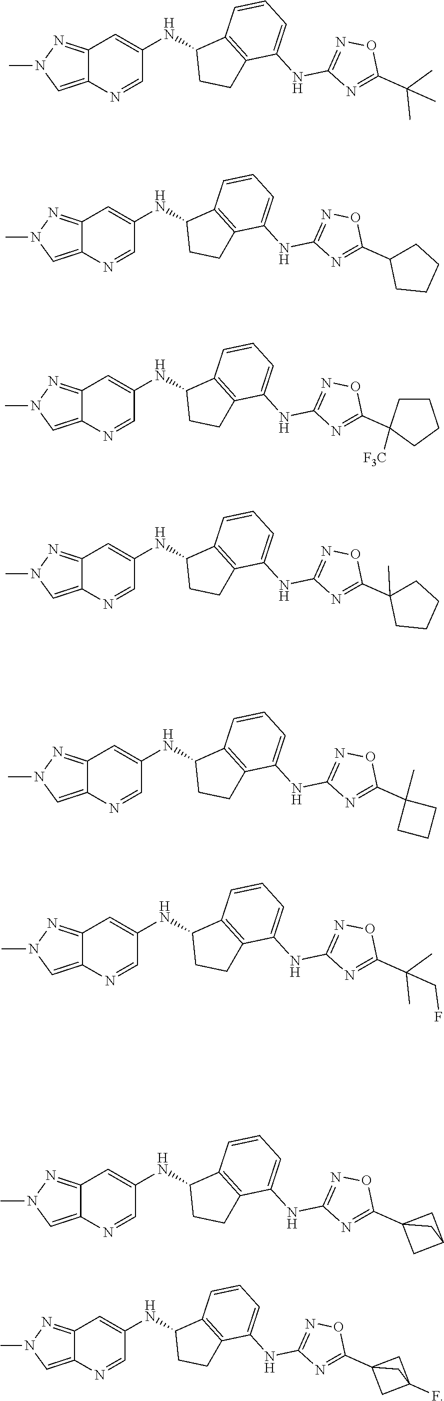

51. The compound according to claim 45 or a pharmaceutically acceptable salt thereof, wherein the compound is selected from:

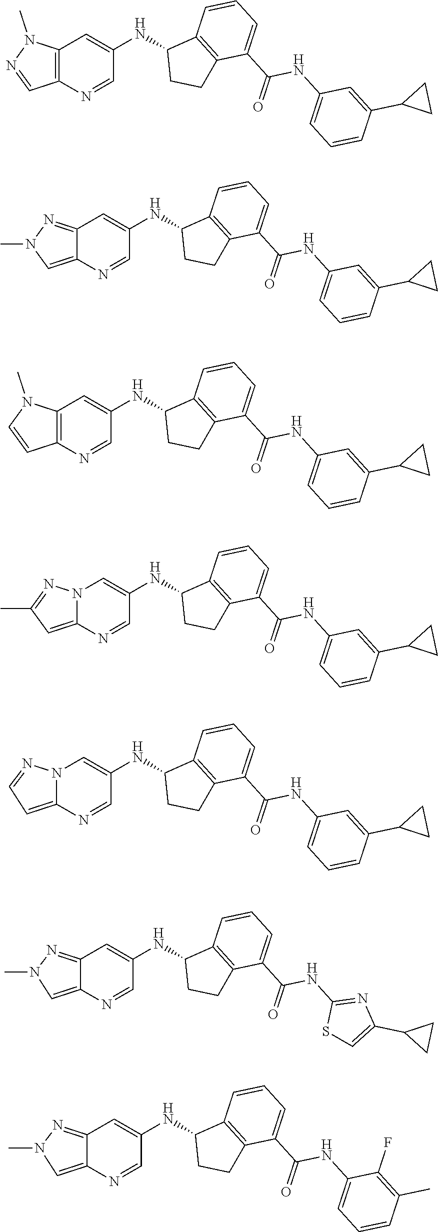

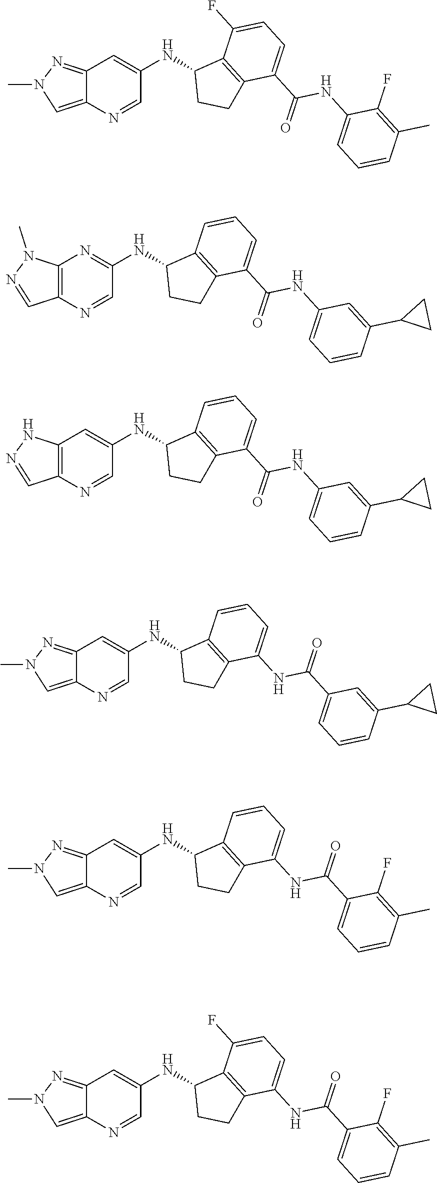

52. The compound according to claim 45 or a pharmaceutically acceptable salt thereof, wherein the compound is selected from:

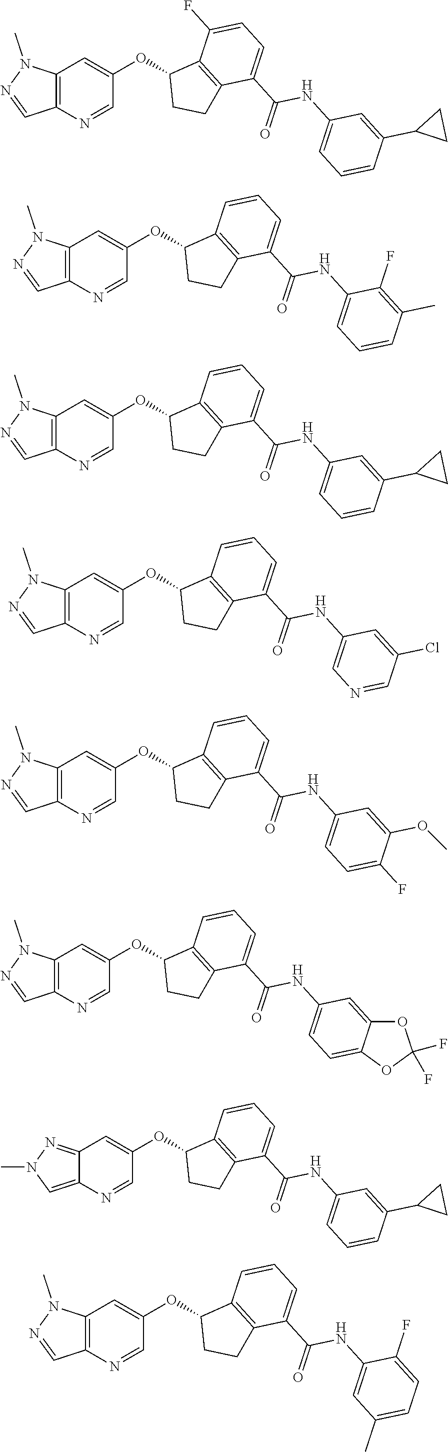

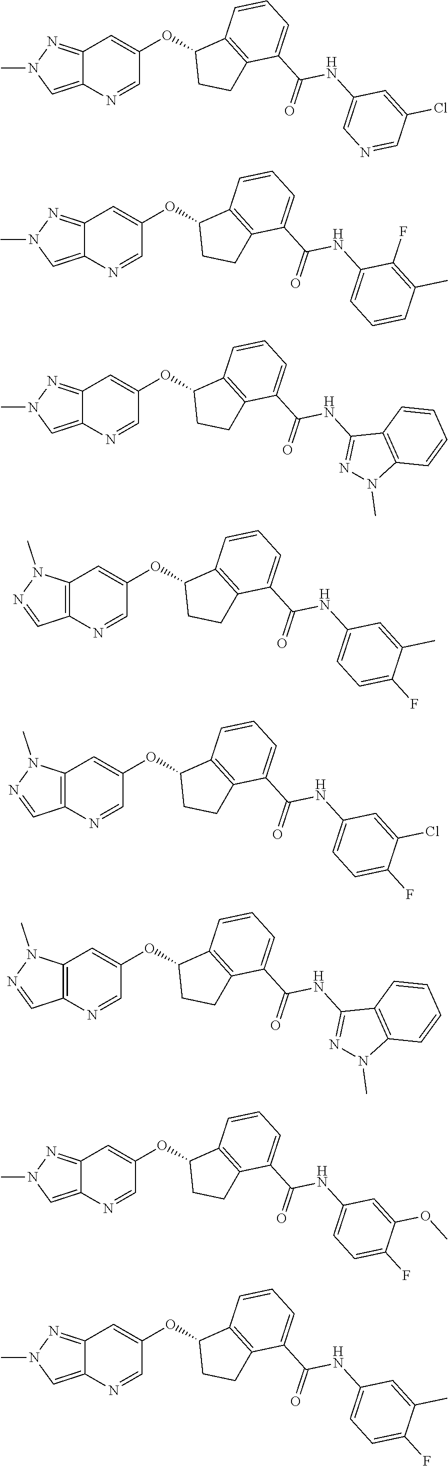

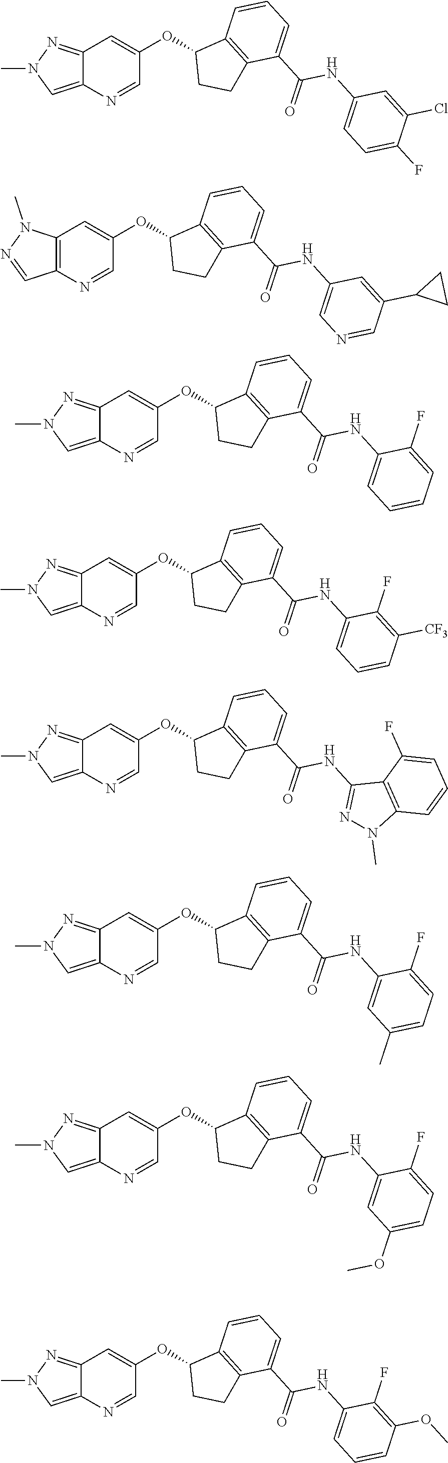

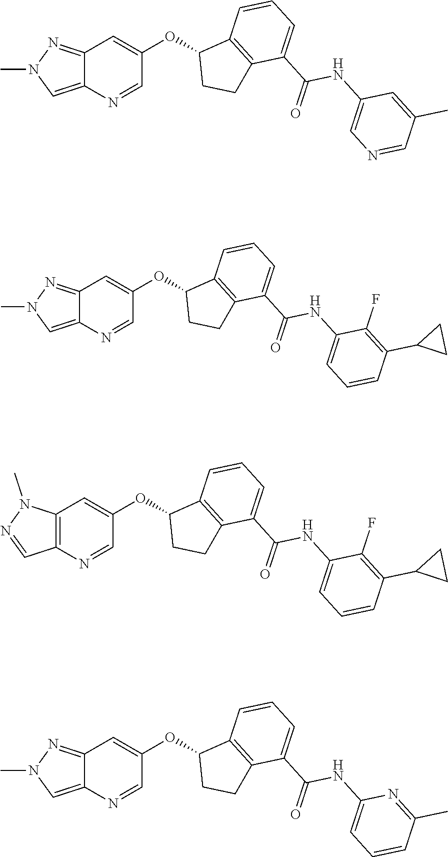

53. A pharmaceutical composition, wherein the composition comprises a compound or a pharmaceutically acceptable salt of formula (I) according to claim 30, or a stereoisomer or an isotopic isomer thereof, and a pharmaceutically acceptable excipient.

54. A method for treating a CSF-1R-related disease in a subject in need thereof, comprising administering to the subject an effective amount of a compound or a pharmaceutically acceptable salt of formula (I) according to claim 30, or a stereoisomer or an isotopic isomer thereof.

55. A pharmaceutical composition, wherein the composition comprises a compound or a pharmaceutically acceptable salt of formula (I) according to claim 45, or a stereoisomer or an isotopic isomer thereof, and a pharmaceutically acceptable excipient.

56. A method for treating a CSF-1R-related disease in a subject in need thereof, comprising administering to the subject an effective amount of a compound or a pharmaceutically acceptable salt of formula (I) according to claim 45, or a stereoisomer or an isotopic isomer thereof.
